(19) United States
(12) Patent Application Publication
(10) Pub. No.: US 2026/0284208 A1
(43) Pub. Date: Sep. 24, 2026

(54) SYSTEM

(71) Applicants: Oxford University Innovation Limited, Oxfordshire (GB); GlaxoSmithKline Intellectual Property Development Limited, Stevenage (GB); The Regents of the University of California, Oakland, CA (US)

(72) Inventors: Patrick Brennan, Oxford (GB); Stuart Conway, Oxford (GB); Charlotte Deane, Oxford (GB); Lewis Brayshaw, Stevenage (GB); Michael Hann, Stevenage (GB)

(21) Appl. No.: 19/167,189

(22) PCT Filed: Mar. 22, 2024
(86) PCT No.: PCT/GB2024/050767

§ 371 Date: Sep. 19, 2025

(30) Foreign Application Priority Data
Mar. 23, 2023 (GB) 2304214.6

(51) Int. Cl. A61K 47/64; A61K 31/454; C07K 14/47

(52) U.S. Cl. CPC A61K 47/64; A61K 31/454; C07K 14/47

(57) ABSTRACT

The present invention relates to novel systems, polypeptides, compounds and methods. The invention provides an inducible degron for target-specific protein degradation.

### FIELD OF THE INVENTION

The present invention relates to novel systems, polypeptides, compounds and methods. The invention particularly provides an inducible degron for target-specific protein degradation.

### BACKGROUND OF THE INVENTION

Chemically induced degradation of proteins has emerged as a powerful complement to traditional, occupancy-based, small molecule therapeutics. This strategy allows all functions of a protein to be neutralized, including scaffolding roles and the action of undruggable domains, which would otherwise be difficult to inhibit. Within this approach, two distinct strategies have been developed to promote proteasomal protein degradation: proteolysis targeting chimeras (PROTACs) and molecular glues.

PROTACs, bifunctional molecules that induce proximity between the target protein of interest (POI) and an E3 ligase to promote POI degradation, have progressed to the clinic as treatments for certain cancers. Alternatively, molecular glues are small molecules that bind to the surface of two proteins, enhancing or inducing their interaction. Molecules that function in this manner include the natural products cyclosporin, FK506, and rapamycin, and synthetic stabilizers of the hub protein 14-3-3 with its binding partners.

The immunomodulatory drugs (IMiDs) thalidomide, lenalidomide, and pomalidomide exert their therapeutic action by stabilizing the interaction between the E3 ligase component cereblon (CRBN) and its zinc finger-containing neosubstrates, Ikaros family zinc finger protein 1 (IKZF1; Ikaros) and Ikaros family zinc finger protein 3 (IKZF3; Aiolos), resulting in the degradation of these proteins. Thalidomide-mediated degradation of the zinc finger-containing transcription factor spalt-like transcription factor 4 (SALL4), again through recruitment of CRBN, is responsible for the infamous teratogenicity caused by thalidomide; pomalidomide also effects degradation of SALL4, while lenalidomide is less effective in this regard. Recent work by Ichikawa et al. and Heim et al. has shown that IMiDs mimick the physiological substrates of CRBN, which comprise C-terminal cyclic imide post-translational modifications, that are formed by intramolecular cyclization of glutamine or asparagine residues.

The zinc finger domains of IKZF1 and IKZF3 are examples of degrons, broadly defined as targeting signals that confer metabolic instability on some, or all, of the peptide bonds in a protein. This definition encompasses peptide sequences that enable substrate recognition by cognate E3 ubiquitin ligases, potentially after post-translational modification, and inducible degrons, which require the presence of a small molecule to promote protein degradation. The latter have been used as a complementary approach to PROTACs, allowing induced degradation of proteins of interest (POIs) that lack a small molecule ligand. Key examples of inducible degrons are HaloPROTACs, dTAG, and BromoTag. HaloPROTACs covalently attach a chloroalkane-linked E3 ligase ligand to a POI-HaloTag fusion resulting in recruitment of the E3 ligase, ubiquitination, and degradation of this fusion protein. The dTAG system employs a fusion of the POI to the FKBP12^{F36V} mutant. A bifunctional molecule comprising a selective FKBP12^{F36V} ligand linked to a CRBN recruiter is used to induce degradation of the POI-FKBP12^{F36V} fusion protein. BromoTag works in a similar fashion, but employs the BRD4(2)^{L387A} mutant fused to the POI. A bifunctional molecule comprising a JQ1-based ligand (AGB1) that binds selectively to BRD4(2)^{L387A}, linked to a VHL ligand, is used to induce degradation of the POI-BRD4(2)^{L387A} fusion protein. These technologies are powerful tools, but they all require the addition of relatively large protein domains to the POI, which has the potential to perturb the endogenous function of the POI.

Zinc finger-based degrons offer an attractive alternative as inducible degrons, as the zinc finger motif typically comprises under 60 amino acids and will therefore minimally perturb the POI. Jan et al. employed mutant combinations of zinc finger motifs to identify super-degron tags that were degraded effectively by lenalidomide. These tags were attached to chimeric antigen receptors (CARs) to enable inducible degradation of the CAR in T-cell on the addition of lenalidomide. The combination of the degron-tagged CARs and lenalidomide functioned as an off switch to limit cytokine production while retaining antitumor effects. Carbonneau took a similar approach to develop a ligand-controlled CAR system using an IKZF3-based degron that was degraded upon addition of lenalidomide.

While approaches such as these have shown some benefits in terms of overcoming the limitations of other inducible degrons by using the relatively small zinc-finger-based degron, problems remains. A particular issue in this regard is off-target degradation induced by the administered compounds. For example, Sievers et al. identified 11 zinc finger-containing transcription factors that were degraded in the presence of thalidomide, lenalidomide, or pomalidomide. Computational analysis suggested that more than 150 zinc fingers can bind to the drug-CRBN complex. Therefore, the use of IMiDs that can bind to the wild-type zinc finger degrons in CAR T-cell therapy, or target validation, risks degradation of additional neosubstrates leading to unwanted effects.

The invention provides a revolutionary new system for combining a polypeptide and a modified IMiD that may function as an inducible degron without affecting other wild type degrons. The present invention may provide for i.a. reduced off-target effects and/or improved targeting, and, in therapeutic applications, may provide for reduced concentrations of therapeutic agents to be required in order to exert a desired therapeutic outcome, with benefits in terms of reduced toxicity, decreased costs, and/or improved patient compliance.

### SUMMARY OF THE INVENTION

The inventors have devised a novel principle of general application which combines a polypeptide degron and a modified IMiD compound for utilisation as an inducible degron. Advantageously, the present invention provides an inducible degron with improved specificity relative to known degron systems.

Surprisingly, the present system can be utilised in cells without inducing the degradation of wild type degrons. This renders the system particularly suitable for use in clinical applications. The inventors have discovered novel polypeptides and novel compounds which together may form an orthogonal protein degradation system. The polypeptide degron can be attached to a protein of interest to selectively induce the degradation of the target protein in cells. To induce the degradation of the target protein of interest, the attached degron polypeptide is complexed with the novel compound which evokes a stable interaction between the E3 ligase component cereblon (CRBN) and the polypeptide. This interaction brings CRBN and the polypeptide into proximity with one another. Accordingly, CRBN catalyses polyubiquitination of the polypeptide which triggers the proteasomal degradation of the polypeptide and the target protein of interest that is attached to said polypeptide.

Accordingly, the system provided herein allows unprecedented selectivity when seeking to induce the degradation of specific target proteins. This provides the user with the power to functionally characterise specific proteins without the occurrence of off-target effects.

Furthermore, when applied to the clinic, the present invention allows the modulation of levels of specific proteins in genetically engineered cells without unwanted off-target effects.

The invention provides a system, comprising:
i) a polypeptide comprising a variant of the amino acid sequence of SEQ ID NO: 1, wherein the variant comprises a modification at the position corresponding to Q147 of SEQ ID NO: 2;
   and
ii) a compound of Formula (A)

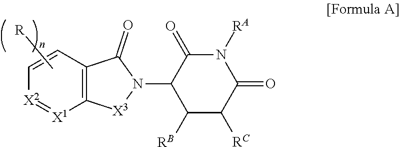

wherein
   X¹ is C—R and X² is C—R or N; or X¹ is C—R or N and X² is C—R;
   X³ is CHR^{D} or C═O;
   each R, which may be the same or different, is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, 5- to 10-membered heteroaryl, 5- to 10-membered heterocyclyl, C₅₋₁₀ carbocyclyl, OR²⁰, N(R²⁰)₂ and halogen;
   and wherein each aryl, heteroaryl, heterocyclyl and carbocyclyl group is independently unsubstituted or is substituted with 1, 2 or 3 substituents independently selected from R²⁰, OR²⁰, N(R²⁰)₂ and halogen;
   each R¹⁰, which may be the same or different, is independently H or C₁₋₄ alkyl which is unsubstituted or substituted with 1, 2 or 3 substituents independently selected from OR²⁰, N(R²⁰)₂ and halogen;
   n is an integer from 0 to 2;
   R^{A} is H or C₁₋₄ alkyl which is unsubstituted or is substituted with 1, 2 or 3 substituents independently selected from OR²⁰, N(R²⁰)₂ and halogen;
   R^{B} is H or C₁₋₄ alkyl which is unsubstituted or is substituted with 1, 2 or 3 substituents independently selected from OR²⁰, N(R²⁰)₂ and halogen;
   R^{C} is H or C₁₋₄ alkyl which is unsubstituted or is substituted with 1, 2 or 3 substituents independently selected from OR²⁰, N(R²⁰)₂ and halogen;
   R^{D} is H or C₁₋₄ alkyl which is unsubstituted or is substituted with 1, 2 or 3 substituents independently selected from OR²⁰, N(R²⁰)₂ and halogen;
   and
   each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl.

The invention further provides a polypeptide comprising a variant of the amino acid sequence of SEQ ID NO: 1, wherein the variant comprises (i) a modification at the position corresponding to Q147 of SEQ ID NO: 2; and (ii) a modification at one or more of the positions corresponding to N149, Q150 and S154 of SEQ ID NO: 2.

The invention further provides a compound of Formula (B),

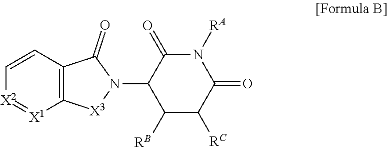

wherein
   X¹ is C—R¹ and X² is C—R² or N; or X¹ is C—R¹ or N and X² is C—R²;
   X³ is CHR^{D} or C═O;
   A:
      R¹ is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl; and
      R² is selected from N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
   or
   B:
      R¹ is selected from OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl; and
      R² is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, 5- to 10-membered heteroaryl, 5- to 10-membered heterocyclyl, C₅0.10 carbocyclyl, OR²⁰, N(R²⁰)₂ and halogen;
      and wherein each aryl, heteroaryl, heterocyclyl and carbocyclyl group is independently unsubstituted or is substituted with 1, 2 or 3 substituents independently selected from R²⁰, OR²⁰, N(R²⁰)₂ and halogen;
   R^{A} is H or C₁0.4 alkyl which is unsubstituted or is substituted with 1, 2 or 3 substituents independently selected from OR²⁰, N(R²⁰)₂ and halogen;
   R^{B} is H or C₁0.4 alkyl which is unsubstituted or is substituted with 1, 2 or 3 substituents independently selected from OR²⁰, N(R²⁰)₂ and halogen;
   R^{C} is H or C₁0.4 alkyl which is unsubstituted or is substituted with 1, 2 or 3 substituents independently selected from OR²⁰, N(R²⁰)₂ and halogen;
   R^{D} is H or C₁0.4 alkyl which is unsubstituted or is substituted with 1, 2 or 3 substituents independently selected from OR²⁰, N(R²⁰)₂ and halogen; and
   each R¹⁰, which may be the same or different, is independently H or C₁0.4 alkyl which is unsubstituted or substituted with 1, 2 or 3 substituents independently selected from OR²⁰, N(R²⁰)₂ and halogen;
   each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl.

The invention further provides a method for degrading a target protein of interest in a cell, the method comprising:
a) introducing a polynucleotide according to the invention, or a vector according to the invention into the cell such that the cell expresses the polypeptide attached to the target protein of interest;
b) contacting the cell with a compound according to the invention;
   thereby inducing the degradation of the target protein of interest.

The invention further provides a compound according to the invention for use in medicine.

The invention further provides a method for identifying an orthogonal IMiD compound and polypeptide degron combination, the method comprising screening for increased binding affinity of one or more compounds according to the invention or as defined in the system of the invention against a polypeptide variant of SEQ ID NO: 2 or SEQ ID NO: 3 relative to a polypeptide sequence consisting of or comprising the polypeptide sequence of SEQ ID NO: 2 or SEQ ID NO: 3, thereby identifying an orthogonal IMiD compound and polypeptide degron combination.

### BRIEF DESCRIPTION OF THE FIGURES

FIG. 1. A. Endogenous neosubstrates IKZF1 and IKZF3 contain a conserved zinc finger region which confers sensitivity to degradation upon treatment with an IMID compound such as thalidomide (pictured). This region can be fused to a protein of interest (POI) to act as a degron; addition of an IMID will lead to degradation of the POI by the proteosome via recruitment of the ubiquitin E3 ligase cereblon. However, use of familiar IMIDs such as thalidomide, lenalidomide or pomalidomide will also lead to degradation of endogenous IKZF1/IKZF3 B Akin to ‘bump-and‘hole’ methodology, we synthesised IMIDs with additional chemical groups on the phthalimide ring and screened these against a library of modified zinc finger degrons, leading to the discovery of two novel, orthogonal IMID/degron systems, for which the IMID compounds do not degrade IKZF1/IKZF3 even at high concentrations. C This region of crystal structure 6HOF shows the IMID pomalidomide acting as a molecular glue between cereblon (pink) and an IKZF1-derived zinc finger (grey) D A different angle of this region highlights the four residues around which our library is primarily based: Q147; N149; Q150; and S154. Q147 is the only ZF residue in direct contact with the IMID and is the most suitable candidate for mutation for the ‘bump-and-hole’ approach; the other residues were chosen for their proximity to residue 147 and the potential cereblon binding-enhancing effects mutation at these positions may convey.

FIG. 2. A. A plasmid containing a ZF degron fused to GFP in the same gene as mCherry. Jurkats that have been lentivirally transduced with this plasmid will express ZF-GFP and mCherry at a consistent ratio. B Addition of an IMID such as pomalidomide will induce GFP degradation via the degron's functionality. C Incubation with an IMID produces a degradation curve.

FIG. 3. A Thalidomide, pomalidomide and a library of 34 other IMIDs were synthesised and screened against the wild-type IKZF1/IKZF3 ZF degron sequence (WT) in jurkat cells (see FIG. 2 for assay description) to obtain pDC50 values. The same compound library was then screened against a single residue mutant ZF degron (Q147A) using the same technique to obtain a second set of pDC50 values. Rejecting all compounds with a Q147A DMax value >=0.4, an orthogonality index value was given to each compound (10\hat{ }(pDC50_Q147A/pDC50_WT)), where a value >10 indicates improved degradation of the Q147A mutant ZF B Two compounds in particular—A63 and B08—can be seen to exhibit improved degradation against Q147A—evidence of the ‘bump-and-hole’ effect, as the smaller alanine residue accommodates the bulky group added to the thalidomide scaffold. C Orthogonality index values for all library compounds with Q147A Dₘₐₓ value <=0.4. For reference, the 36 numbered compounds correspond to the IMID analogues depicted in FIG. 10, and thus may thus alternatively be named herein according to their IMiD analogue names in FIG. 10. The 36 numbered compounds and their corresponding IMiD analogue names are also set out as follows:

| Compound no. | IMiD analogue name |
|---|---|
| 1 | A32 |
| 2 | A03 |
| 3 | A48 |
| 4 | A17 |
| 5 | A75 |
| 6 | B05 |
| 7 | A87 |
| 8 | A08 |
| 9 | A46 |
| 10 | A89 |
| 11 | A86 |
| 12 | A93 |
| 13 | A52 |
| 14 | B01 |
| 15 | A88 |
| 16 | A13 |
| 17 | A45 |
| 18 | A84 |
| 19 | A63 |
| 20 | B02 |
| 21 | A62 |
| 22 | B03 |
| 23 | B08 |
| 24 | A27 |
| 25 | A28 |
| 26 | A22 |
| 27 | A31 |
| 28 | A34 |
| 29 | A53 |
| 30 | A20 |
| 31 | A43 |
| 32 | A42 |
| 33 | A65 |
| 34 | A83 |
| 35 | A66 |
| 36 | A74 |

FIG. 4. A. Using crystal structure 6HOF, PositionScan functionality in Foldx and PositionMutation functionality in Rosetta were both used to assess impact of mutation at positions on the ZF in direct contact with cereblon: 149, 150, 153 and 167. Favourable mutations were only predicted at positions 149 and 150. Mutations which performed well in both softwares were chosen for our library (D, E and P for 149; I and V for 150) along with the WT residues (N for 149; Q for 150). B Our library—a total of 8,380 mutant ZFs—was synthesised in the same ZF-GFP-IRES-mCherry plasmid as used previously. The library was lentivirally transduced into Jurkat cells, ensuring a copy number of 1 per cell. The library population was incubated at an appropriate concentration of IMID, incubated overnight, then sorted by FACS to isolate low GFP cells. This, and the negative population, were sequenced and the ratio of each mutant in the positive and negative population used to determine level of degradation. C Volcano plots show ratio between negative and positive populations for each mutant (log₂FC), with mutants overrepresented in the GFP-negative population appearing as ‘hits’. WT sequence is circled. Red points indicate mutants which were also ‘hits’ in a DMSO control FACS sort. D Average log FC for all mutants with different residues at position 147, all possible mutations at 149, 150 and 154 (included in the library), and all other positions same as WT. E Mean log FC for all mutants with different residues at 149 and 150, all possible mutations at 147 and 154 (included in the library), and all other positions same as WT. F Mean log FC for all mutants with different residues at 154, all possible mutations at 147, 149 and 150 (included in the library), and all other positions same as WT.

FIG. 5. Heat map showing Log FC values from library screen for mutants with all combinations of residues at 147 (all except P), 149 (D, E, N and P), 150 (I, Q and V) and 154 (all 20) after treatment with either thalidomide, A63 or B08.

FIG. 6. A Top performing mutants from the library screen (top 5 highest performing log FC values for A63 screen; 1st, 3rd and 4th highest scoring mutants for B08) were transduced into Jurkat cells and assessed using the same flow cytometry procedure as previously with their corresponding IMIDs to obtain pDC₅₀ values. Mutants are named after their residues at positions 147, 149, 150 and 154 (e.g. WT would be named QNQS) (all other residues are same as WT). Each IMID/mutant pair is assigned an orthogonality index value as in FIG. 3. B Graphical representation of orthogonality index values for top performing mutants for A63 and B08. The top performing mutant for A63 is AEVK and for B08 is WEIS C Concentration curves for flow cytometry assay confirms close to 100% degradation of the top mutant ZF-GFP at 10 μM for both A63 and B08. WT ZF-GFP is degraded 60-70%, in contrast to the endogenous IKZF3, which is unaffected relative to DMSO control at 1 μM or 10 μM for both A63 and B08—as seen in D using Western blot analysis of untransduced Jurkat cells treated with pomalidomide, A63, B08. E Western blot for Jurkats transduced with AEVK-GFP shows full degradation of AEVK-GFP at 10 μM A63, while level of endogenous IKZF3 remains unaffected.

FIG. 7. 41 proposed compounds were docked into a region of crystal structure 6H0F in place of pomalidomide. Constrained docking in GOLD was used to hold the piperidine-2,6-dione component of the IMID inside the cereblon binding pocket, while allowing the phthalimide bicyclic ring and any group on position 4 or 5 to be flexible. Foldx was used to mutate position 147 on the zinc finger to all 20 amino acids, and GOLD allowed this residue to be flexible. The resulting ternary complex poses were then analysed using the scoring function from Autodock4 to provide a compound ranking. A particularly strong predicted pi-pi interaction between the benzyl group in thal_4_NH_CH2_C6H5 and tryptophan 147 in the Q147W ZF was detected using this screen.

FIG. 8. A A virtual library of thal_4_NH_CH2_C6H5 analogues was screened against the Q147W zinc finger in the same constrained docking protocol as described in FIG. 7, highlighting two modifications (ortho-methyl and 3-pyridyl) for which the difference in estimated binding energy between WT and Q147W is predicted to be the greatest (B). C The proposed binding pose for thal_4_NH_CH2_C6H5 docked into the cereblon/ZF binary complex (allowing the side chain of residue 147 to be flexible) clearly predicted a favourable pi-pi interaction between the R group of IMID and the side chain of W147.

FIG. 9. A The seven ZF degron residues chosen to scrutinise most thoroughly in the Jurkat library screen are shown: Q147 is proximal to the IMID and S154 interacts with Q147; N149 and Q150 are closed to the IMID; F146, A153 and F155 comprise the ‘hydrophobic core’ of the ZF motif. B The design of the ZF degron library is summarised: part 1 makes up the bulk of the library, where a full combinatorial exploration of choice mutations at positions 147, 149, 150 and 154 are explored, keeping all other residues the same as the WT ZF; part 2 explores subsections of the mutations seen in part 1, but with additional mutations to positions 146, 153 and 155; part 3 explores changes to 147 and a single change of residue 167 with all else being kept the same as the WT; part 4 is a single sequence where all is kept the same as WT except the glycine at 152.

FIG. 10. A 36 IMID analogues were screened against the WT ZF using a GFP/mCherry fluorescence flow cytometry assay to give degradation curves B The same 36 IMID analogues were screened in the same assay against a Q147A mutant degron to give new degradation curves. For reference, the 36 IMID analogues of FIG. 10 may be alternatively named herein according to their corresponding compound no. 1 to 36 as follows:

| Compound no. | IMiD analogue name |
|---|---|
| 1 | A32 |
| 2 | A03 |
| 3 | A48 |
| 4 | A17 |
| 5 | A75 |
| 6 | B05 |
| 7 | A87 |
| 8 | A08 |
| 9 | A46 |
| 10 | A89 |
| 11 | A86 |
| 12 | A93 |
| 13 | A52 |
| 14 | B01 |
| 15 | A88 |
| 16 | A13 |
| 17 | A45 |
| 18 | A84 |
| 19 | A63 |
| 20 | B02 |
| 21 | A62 |
| 22 | B03 |
| 23 | B08 |
| 24 | A27 |
| 25 | A28 |
| 26 | A22 |
| 27 | A31 |
| 28 | A34 |
| 29 | A53 |
| 30 | A20 |
| 31 | A43 |
| 32 | A42 |
| 33 | A65 |
| 34 | A83 |
| 35 | A66 |
| 36 | A74 |

FIG. 11. A 13 IMID analogues were incubated with unmodified Jurkat cells at different concentrations for 20 hours, after which cells were fixed and treated with IKZF1-specific antibody and analysed by flow cytometry. B Mutant ZF degron sequences with the highest log FC values from the A63 and B08 library screens were selected for individual analysis by the same GFP/mCherry assay as described in FIG. 10 along with others chosen for proof of concept.

FIG. 12. A Jurkat cells transduced with WT ZF-eGFP constructs were treated with pomalidomide at 1 nM, 10 nM or 100 nM for 20 hours, then Western Blots performed on the cell lysates. B Untransduced Jurkat cells were incubated with pomalidomide, A63 and B08 (all at 100 nM, 1p M or 10 μM) for 20 hours and Western Blots performed on the cell lysates. C Jurkat cells transduced with HEIP ZF-eGFP constructs were treated with A63 at 1 nM, 10 nM, 100 nM, 1 M or 10 μM for 20 hours, then Western Blots performed on the cell lysates. D Jurkat cells transduced with AEVK ZF-eGFP constructs were treated with A63 at 1 nM, 10 nM, 100 nM, 1 μM or 10 μM for 20 hours, then Western Blots performed on the cell lysates E Jurkat cells transduced with WEIS ZF-eGFP constructs were treated with B08 at 1 nM, 10 nM, 100 nM, 1 μM or 10 μM for 20 hours, then Western Blots performed on the cell lysates.

FIG. 13. Average fold enrichment and depletion of sequencing read counts and corresponding P values for sequences with raw read count >xxx following compound treatments. Three experimental replicates. WT sequence is circled. Red points indicate sequences significantly enriched following DMSO treatment.

FIG. 14. A Correlation of sorted positive and negative samples in the pooled library screen. B Limma-pi values of enriched degrons for each compound treatment in the pooled library screen.

FIG. 15. A schematic depicting a system combining a mutant degron polypeptide and a ‘bumped’ (i.e. modified) IMiD. The bumped IMiD cannot bind wild type degrons and therefore does not induce the degradation said wild type degrons. The bumped IMiD binds the mutant degron and CRBN and thereby indices the degradation of the mutant degron and any associated protein.

FIG. 16. Heat map shows Log FC values from library screen for mutants with all combinations of residues at stated positions of SEQ ID NO: 2 after treatment with either thalidomide, lenalidomide, as well as compounds 19, 22 and 23, relative to DMSO treatment.

FIG. 17. The 36 IMID analogues of FIG. 10 reproduced wherein each compound is annotated with its compound number 1 to 36 and alternative name.

FIG. 18. Testing of four additional compounds, B22 (compound 37), B25 (compound 38), B31 (compound 39) and B32 (compound 40) as described in example 2.

FIG. 19. Testing of an additional compound C01 as described in example 3.

### BRIEF DESCRIPTION OF THE SEQUENCES

polypeptide sequence
SEQ ID NO: 1
FQCNQCGASF
IKZF3 polypeptide sequence
SEQ ID NO: 2
MEDIQTNAELKSTQEQSVPAESAAVLNDYSLTKSHEMENVDSGEGPAN
EDEDIGDDSMKVKDEYSERDENVLKSEPMGNAEEPEIPYSYSREYNEY
ENIKLERHVVSFDSSRPTSGKMNCDVCGLSCISFNVLMVHKRSHTGER
PFQCNQCGASFTQKGNLLRHIKLHTGEKPFKCHLCNYACQRRDALTGH
LRTHSVEKPYKCEFCGRSYKQRSSLEEHKERCRTFLQSTDPGDTASAE
ARHIKAEMGSERALVLDRLASNVAKRKSSMPQKFIGEKRHCFDVNYNS
SYMYEKESELIQTRMMDQAINNAISYLGAEALRPLVQTPPAPTSEMVP
VISSMYPIALTRAEMSNGAPQELEKKSIHLPEKSVPSERGLSPNNSGH
DSTDTDSNHEERQNHIYQQNHMVLSRARNGMPLLKEVPRSYELLKPPP
ICPRDSVKVINKEGEVMDVYRCDHCRVLFLDYVMFTIHMGCHGFRDPF
ECNMCGYRSHDRYEFSSHIARGEHRALLK
IKZF1 polypeptide sequence
SEQ ID NO: 3
MDADEGQDMSQVSGKESPPVSDTPDEGDEPMPIPEDLSTTSGGQQSSK
SDRVVASNVKVETQSDEENGRACEMNGEECAEDLRMLDASGEKMNGSH
RDQGSSALSGVGGIRLPNGKLKCDICGIICIGPNVLMVHKRSHTGERP
FQCNQCGASFTQKGNLLRHIKLHSGEKPFKCHLCNYACRRRDALTGHL
RTHSVGKPHKCGYCGRSYKQRSSLEEHKERCHNYLESMGLPGTLYPVI
KEETNHSEMAEDLCKIGSERSLVLDRLASNVAKRKSSMPQKFLGDKGL
SDTPYDSSASYEKENEMMKSHVMDQAINNAINYLGAESLRPLVQTPPG
GSEVVPVISPMYQLHKPLAEGTPRSNHSAQDSAVENLLLLSKAKLVPS
EREASPSNSCQDSTDTESNNEEQRSGLIYLTNHIAPHARNGLSLKEEH
RAYDLLRAASENSQDALRVVSTSGEQMKVYKCEHCRVLFLDHVMYTIH
MGCHGFRDPFECNMCGYHSQDRYEFSSHITRGEHRFHMS

### DETAILED DESCRIPTION OF THE INVENTION

The present invention will be described with respect to particular embodiments and with reference to certain drawings but the disclosure is not limited thereto but only by the claims. Any reference signs in the claims shall not be construed as limiting the scope. Of course, it is to be understood that not necessarily all aspects or advantages may be achieved in accordance with any particular embodiment. Thus, for example those skilled in the art will recognize that the disclosed embodiments may be embodied or carried out in a manner that achieves or optimizes one advantage or group of advantages as taught herein without necessarily achieving other aspects or advantages as may be taught or suggested herein.

The disclosure, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one disclosed embodiment. Thus, appearances of the phrases “in one embodiment” or “in an embodiment” in various places throughout this specification are not necessarily all referring to the same embodiment, but may. Similarly, it should be appreciated that in the description of exemplary disclosed embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

It should be appreciated that “embodiments” of the disclosure can be specifically combined together unless the context indicates otherwise. The specific combinations of all disclosed embodiments (unless implied otherwise by the context) are further disclosed embodiments of the claimed invention.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a protein” includes two or more proteins, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA).

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide is typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypeptides that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution or deletion of one or more amino acids.

A “variant” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the present invention, a “variant” has at least 50%, 60%, 70%, 80%, 90%, 95% or 99% complete sequence identity to the amino acid sequence of the corresponding wild-type protein. Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product.

Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 6 below. Where amino acids have similar polarity, this can also be determined by reference to the hydropathy scale for amino acid side chains in Table 7.

TABLE 6
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 7
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

Unless otherwise indicated, nucleic acid sequences herein are written in the 5′-to-3′ direction from left to right.

As used herein, a C₁₋₆ alkyl group is a linear or branched alkyl group containing from 1 to 6 carbon atoms. A C₁₋₄ alkyl group is a linear or branched alkyl group containing from 1 to 4 carbon atoms. A C₁₋₄ alkyl group is often a C₁₋₃ alkyl group or a C₁₋₂ alkyl group. Examples of C₁₋₄ alkyl groups include methyl, ethyl, n-propyl, iso-propyl, n-butyl, iso-butyl, sec-butyl, and tert-butyl. A C₁₋₂ alkyl group is methyl or ethyl, typically methyl. For the avoidance of doubt, where two alkyl groups are present, the alkyl groups may be the same or different.

As used herein, a C₁₋₄ alkoxy is a C₁₋₄ alkyl group as defined above joined to an oxygen atom.

As used herein, a C₂₋₆ alkenyl group is a linear or branched alkyl group containing from 2 to 6 carbon atoms and having one or more, e.g. one or two, typically one double bonds. A C₂₋₆ alkenyl group is often a C₂₋₄ alkenyl group. Typically a C₂₋₄ alkenyl group is a C₂₋₃ alkenyl group. Examples of C₂₋₄ alkenyl groups include ethenyl, propenyl and butenyl.

As used herein, a C₂₋₆ alkynyl group is a linear or branched alkyl group containing from 2 to 6 carbon atoms and having one or more, e.g. one or two, typically one triple bonds. A C₂₋₆ alkynyl group is often a C₂₋₄ alkynyl group. Typically a C₂₋₄ alkynyl group is a C₂₋₃ alkynyl group, for example ethynyl, propynyl or butynyl.

An alkyl, alkoxy, alkenyl, or alkynyl group as used herein may be unsubstituted or substituted. Unless otherwise stated, substituted alkyl, alkoxy, alkenyl, or alkynyl groups typically carry one or more, e.g. 1, 2, 3 or 4, such as one, two or three e.g. one or two substituents. Preferred substituents include aryl, heteroaryl, heterocyclyl, alkoxy, amino and halogen substituents. The substituents on a substituted alkyl, alkoxy, alkenyl or alkynyl group are typically themselves unsubstituted unless otherwise stated. Where more than one substituent is present, these may be the same or different.

As used herein, a halogen is typically chlorine, fluorine, bromine or iodine and is preferably chlorine, bromine or fluorine, especially chorine or fluorine, and most especially fluorine. Where a group or moiety is substituted with one or more halogen atoms, preferably it carries one, two, three or four halogen atoms, preferably one, two or three halogen atoms. Where a group or moiety is substituted with two or more halogen atoms, the halogen atoms may be the same or different. Typically, the halogen atoms are the same.

As used herein a 6- to 10-membered aryl group is a substituted or unsubstituted, monocyclic or fused polycyclic aromatic group containing from 6 to 10 carbon atoms in the ring portion. Examples include monocyclic groups such as phenyl and fused bicyclic groups such as naphthyl and indenyl. Phenyl (benzene) is preferred. As used herein, a C₆₋₁₀ aryl group is typically a 6- to 10-membered aryl group as defined herein.

As used herein, a 5- to 10-membered heteroaryl group is a substituted or unsubstituted, monocyclic aromatic group containing from 5 to 10 atoms in the ring portion, including at least one heteroatom, for example 1, 2 or 3 heteroatoms, typically selected from O, S and N; and is typically a 5- to 6-membered heteroaryl group which is a substituted or unsubstituted, monocyclic aromatic group containing 5 or 6 atoms in the ring portion, including at least one heteroatom, for example 1, 2 or 3 heteroatoms, typically selected from O, S and N. Examples of 5- and 6-membered heteroaryl groups include pyrrole, furan, thiophene, imidazole, oxazole, thiazole, pyridine, pyridazine, pyrimidine, and pyrazine. As used herein, a C₅₋₁₀ heteroaryl group is typically a 5- to 10-membered heteroaryl group as defined herein. As used herein, a C₅₋₆ heteroaryl group is typically a 5- to 6-membered heteroaryl group as defined herein.

As used herein, a 5- to 10-membered heterocyclyl group is a cyclic group containing from 5 to 10 atoms selected from C, O, N and S in the ring, including at least one heteroatom, and typically one or two heteroatoms; and is typically a 5- to 8-membered heterocyclyl group, typically a 5- to 6-membered heterocyclyl group which is a cyclic group containing from 5 to 6 atoms selected from C, O, N and S in the ring, including at least one heteroatom, and typically one or two heteroatoms. The heteroatom or heteroatoms are typically selected from O, N, and S. A heterocyclic group may be saturated or partially unsaturated. A 5- to 6-membered partially unsaturated heterocyclic group is a cyclic group containing from 5 to 6 atoms selected from C, O, N and S in the ring and containing 1 or 2, e.g. 1 double bond. As used herein, a C₅₋₁₀ heterocyclyl group is typically a 5- to 10-membered heterocyclyl group as defined herein. As used herein, a C₅₋₆ heterocyclyl group is typically a 5- to 6-membered heterocyclyl group as defined herein.

A 5- to 10-membered carbocyclyl group is a cyclic hydrocarbon containing from 5 to 10 carbon atoms. A carbocyclyl group may be saturated or partially unsaturated, but is typically saturated. A 5- to 10-membered partially unsaturated carbocyclyl group is a cyclic hydrocarbon containing from 5 to 10 carbon atoms and containing 1 or 2, e.g. 1 double bond. A 5- to 10-membered carbocyclyl group is typically a 5- to 8-membered carbocyclyl group such as a 5- to 6-membered carbocyclic group. Examples of 5- to 6-membered saturated carbocyclic groups include cyclopentyl and cyclohexyl groups. As used herein, a C₅₋₁₀ carbocyclyl group is typically a 5- to 10-membered carbocyclyl group as defined herein. As used herein, a C₅₋₆ carbocyclyl group is typically a 5- to 6-membered carbocyclyl group as defined herein.

Examples of 5- to 6-membered saturated heterocyclic groups include piperazine, piperidine, morpholine, pyrrolidine, imidazolidine, and oxazolidine. Examples of 5- to 6-membered partially unsaturated heterocyclic groups include tetrahydropyrazine, tetrahydropyridine, dihydro-1,4-oxazine, tetrahydropyrimidine, dihydro-1,3-oxazine, dihydropyrrole, dihydroimidazole and dihydrooxazole.

An aryl, heterocyclyl or heteroaryl group may be unsubstituted or substituted as described herein. For example, an aryl, heterocyclyl or heteroaryl group may be unsubstituted or substituted with 1, 2 or 3, typically 1 or 2 such as e.g. 1 substituent. Suitable substituents include alkyl group, alkoxyl groups, alkenyl groups, alkynyl groups, halogen, OH, and amino groups, which are typically themselves unsubstituted or substituted with one or more halogen atoms.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as oxalic, citric, fumaric, maleic, malic, ascorbic, succinic, tartaric, benzoic, acetic, methanesulphonic, ethanesulphonic, benzenesulphonic or p-toluenesulphonic acid.

Pharmaceutically acceptable bases include alkali metal (e.g. sodium or potassium) and alkaline earth metal (e.g. calcium or magnesium) hydroxides and organic bases such as alkyl amines, aralkyl amines and heterocyclic amines. Preferred pharmaceutically acceptable salts are salts formed at the SO₃H group with pharmaceutically acceptable bases, in particular quaternary ammonium salts e.g. tetrabutylammonium salts, or alkali metal salts, e.g. sodium or potassium salts, most preferably sodium salts.

All publications, patents and patent applications cited herein, whether supra or infra, are hereby incorporated by reference in their entirety.

### System

As explained in more detail herein, provided herein is a system. A system may for example be a biological system, a combination of components which may be produced, sold and distributed separately which together may optionally form a kit, or simply a combination of physical products.

The system provided herein comprises a polypeptide comprising a variant of the amino acid sequence of SEQ ID NO: 1, as described in more detail herein. The system further comprises a compound of Formula (A) as defined herein, preferably a compound of Formula (I) as defined herein.

The system may comprise a mutant degron/IMID pair. In some embodiments the compound of formula (A) preferentially binds to the polypeptide. In some embodiments the compound of formula (A) preferentially binds to the polypeptide compared to the binding to native proteins in vivo, such as proteins of SEQ ID NO: 2

The system provided herein comprises:
i) a polypeptide comprising a variant of the amino acid sequence of SEQ ID NO: 1, wherein the variant comprises a modification at the position corresponding to Q147 of SEQ ID NO: 2;
and
ii) a compound of Formula (A)

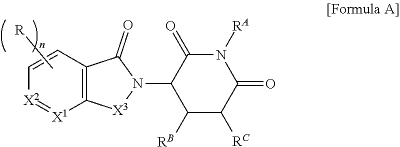

wherein
   X¹ is C—R and X² is C—R or N; or X¹ is C—R or N and X² is C—R;
   X³ is CHR^{D} or C═O;
   each R, which may be the same or different, is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, 5- to 10-membered heteroaryl, 5- to 10-membered heterocyclyl, C₅0.10 carbocyclyl, OR²⁰, N(R²⁰)₂ and halogen;
      and wherein each aryl, heteroaryl, heterocyclyl and carbocyclyl group is independently unsubstituted or is substituted with 1, 2 or 3 substituents independently selected from R²⁰, OR²⁰, N(R²⁰)₂ and halogen;
   each R¹⁰, which may be the same or different, is independently H or C₁₋₄ alkyl which is unsubstituted or substituted with 1, 2 or 3 substituents independently selected from OR²⁰, N(R²⁰)₂ and halogen;
   n is an integer from 0 to 2;
   R^{A} is H or C₁0.4 alkyl which is unsubstituted or is substituted with 1, 2 or 3 substituents independently selected from OR²⁰, N(R²⁰)₂ and halogen;
   R^{B} is H or C₁0.4 alkyl which is unsubstituted or is substituted with 1, 2 or 3 substituents independently selected from OR²⁰, N(R²⁰)₂ and halogen;
   R^{C} is H or C₁0.4 alkyl which is unsubstituted or is substituted with 1, 2 or 3 substituents independently selected from OR²⁰, N(R²⁰)₂ and halogen;
   R^{D} is H or C₁0.4 alkyl which is unsubstituted or is substituted with 1, 2 or 3 substituents independently selected from OR²⁰, N(R²⁰)₂ and halogen; and
   each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl.

Preferably, the system provided herein comprises:
i) a polypeptide comprising a variant of the amino acid sequence of SEQ ID NO: 1, wherein the variant comprises a modification at the position corresponding to Q147 of SEQ ID NO: 2;
and
ii) a compound of Formula (A)

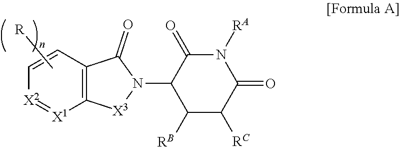

wherein
   X¹ is C—R and X² is C—R or N; or X¹ is C—R or N and X² is C—R;
   X³ is CHR^{D} or C═O;
   each R, which may be the same or different, is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰-C₂₋₆ alkynyl, C₆₋₁₀ aryl, C₅₋₁₀ heteroaryl, C₅₋₁₀ heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, C₅₋₁₀ heteroaryl, C₅₋₁₀ heterocyclyl, C₅₋₁₀ carbocyclyl, OR²⁰, N(R²⁰)₂ and halogen;
      and wherein each aryl, heteroaryl, heterocyclyl and carbocyclyl group is independently unsubstituted or is substituted with 1 or 2 substituents independently selected from R²⁰, OR²⁰, N(R²⁰)₂ and halogen;
   each R¹⁰, which may be the same or different, is independently H or C₁₋₄ alkyl which is unsubstituted or substituted with 1, 2 or 3 substituents independently selected from OR²⁰, N(R²⁰)₂ and halogen;
   n is an integer from 0 to 2;
   R^{A} is H or C₁0.4 alkyl which is unsubstituted or is substituted with 1, 2 or 3 substituents independently selected from OR²⁰, N(R²⁰)₂ and halogen;
   R^{B} is H or C₁0.4 alkyl which is unsubstituted or is substituted with 1, 2 or 3 substituents independently selected from OR²⁰, N(R²⁰)₂ and halogen;
   R^{C} is H or C₁0.4 alkyl which is unsubstituted or is substituted with 1, 2 or 3 substituents independently selected from OR²⁰, N(R²⁰)₂ and halogen;
   R^{D} is H or C₁0.4 alkyl which is unsubstituted or is substituted with 1, 2 or 3 substituents independently selected from OR²⁰, N(R²⁰)₂ and halogen; and
   each R²⁰, which may be the same or different, is independently H or methyl.
      The compound of Formula (A) is preferably a compound of Formula (I):

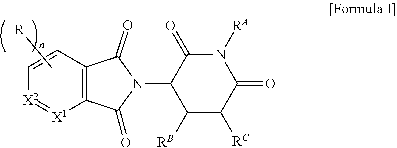

wherein
   X¹ is C—R and X² is C—R or N; or X¹ is C—R or N and X² is C—R;
   each R, which may be the same or different, is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰-C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, C₅₋₁₀ heteroaryl, C₅₋₁₀ heterocyclyl, C₅₋₁₀ carbocyclyl, OR²⁰, N(R²⁰)₂ and halogen;
      and wherein each aryl, heteroaryl, heterocyclyl and carbocyclyl group is independently unsubstituted or is substituted with 1, 2, or 3, preferably 1 or 2 substituents independently selected from R²⁰, OR²⁰, N(R²⁰)₂ and halogen;
   each R¹⁰, which may be the same or different, is independently H or C₁₋₄ alkyl which is unsubstituted or substituted with 1, 2 or 3 substituents independently selected from OR²⁰, N(R²⁰)₂ and halogen;
   n is an integer from 0 to 2;
   R^{A} is H or C₁₋₄ alkyl which is unsubstituted or is substituted with 1, 2 or 3 substituents independently selected from OR²⁰, N(R²⁰)₂ and halogen;
   R^{B} is H or C₁₋₄ alkyl which is unsubstituted or is substituted with 1, 2 or 3 substituents independently selected from OR²⁰, N(R²⁰)₂ and halogen;
   R^{C} is H or C₁₋₄ alkyl which is unsubstituted or is substituted with 1, 2 or 3 substituents independently selected from OR²⁰, N(R²⁰)₂ and halogen; and
   each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl; preferably each R²⁰ is independently H or methyl.

The present inventors have determined that the system provided herein can function as an inducible degron system without affecting wild type degrons. A polypeptide according to the system provided herein, together with a compound of Formula (A) according to the system provided herein, therefore provides the system with a mode of action whereby proteins of interest can be specifically targeted for degradation whilst other proteins, including other degrons, are not targeted for degradation. Such a mode of action can be leveraged, for example, for experimental and therapeutic purposes. The specificity that can be achieved by the system means that off-target activity is minimised, and the potential for unwanted side-effects in a therapeutic setting is reduced.

The polypeptide comprises a variant of the amino acid sequence of SEQ ID NO: 1, wherein the variant comprises a modification at the position corresponding to Q147 of SEQ ID NO: 2. SEQ ID NO: 1 corresponds to amino acids F146-F155 of the polypeptide sequence of IKZF3, which is defined according to SEQ ID NO: 2. Accordingly, in some embodiments the polypeptide comprises a variant of the amino acid sequence of SEQ ID NO: 2, wherein the variant comprises a modification at the position corresponding to Q147 of SEQ ID NO: 2.

IKZF1 is comprised within the same Ikaros zinc finger family as IKZF3 and therefore shares a substantial degree sequence homology to IKZF3. F146-F155 of IKZF3 is identical to F145-F154 in IKZF1. SEQ ID NO: 1 therefore also corresponds to amino acids F145-F154 of the polypeptide sequence of IKZF1, which is defined according to SEQ ID NO: 3. The polypeptide may therefore alternatively comprise a variant of the amino acid sequence of SEQ ID NO: 1, wherein the variant comprises a modification at the position corresponding to Q146 of SEQ ID NO: 3. Accordingly, in some embodiments the polypeptide comprises a variant of the amino acid sequence of SEQ ID NO: 3, wherein the variant comprises a modification at the position corresponding to Q146 of SEQ ID NO: 3. Those skilled in the art will appreciate that the variant may be a variant defined according SEQ ID NO: 3 and thereby comprise modifications at the positions which correspond to the positions of SEQ ID NO: 2 disclosed herein.

The variant of SEQ ID NO: 1 may be defined herein according SEQ ID NO: 2 or SEQ ID NO: 3.

The variant may further comprise a modification at one or more of the positions corresponding to N149, Q150 and S154 of SEQ ID NO: 2. The variant preferably further comprises a modification at the position corresponding to N149 of SEQ ID NO: 2. The variant preferably further comprises a modification at the position corresponding to Q150 of SEQ ID NO: 2. The variant preferably further comprises a modification at the position corresponding to S154 of SEQ ID NO: 2. The variant more preferably further comprises a modification at the position corresponding to N149 and Q150 of SEQ ID NO: 2, N149 and S154 of SEQ ID NO: 2, or Q150 and S154 of SEQ ID NO: 2. The variant may alternatively further comprise modification at the positions corresponding to N149, Q150 and S154 of SEQ ID NO: 2.

The aforementioned modifications have been defined relative to SEQ ID NO: 2. Those skilled in the art will appreciate that the variant may be a variant of SEQ ID NO: 2 and comprise the aforementioned modifications. The variant may be a variant of SEQ ID NO: 3 and comprise the aforementioned modifications relative to SEQ ID NO: 3; for example, the variant may comprise a modification at one or more of the positions corresponding to N148, Q149 and S153 of SEQ ID NO: 3.

The variant may have at least about 50% sequence identity, 60% sequence identity, or preferably at least 70% sequence identity to SEQ ID NO: 1.

Standard methods in the art may be used to determine homology. For example the UWGCG Package provides the BESTFIT program which can be used to calculate homology, for example used on its default settings (Devereux et al. (1984) Nucleic Acids Research 12, p387-395). The PILEUP and BLAST algorithms can be used to calculate homology or line up sequences (such as identifying equivalent residues or corresponding sequences (typically on their default settings)), for example as described in Altschul S. F. (1993) J. Mol. Evol. 36:290-300; Altschul, S. F et al. (1990) J. Mol. Biol. 215:403-10. Software for performing BLAST analyses is publicly available through the National Center for Biotechnology Information (http://www.ncbi.nlm.nih.gov/).

The modification comprised in the variant may be any modification known in the art to alter the chemical properties of an amino acid at a specific position. The modification may particularly alter the chemical properties of the amino acid at the specific position such that the amino acid has different electrostatic or hydrophobic properties, such that its ability to chemical interact with a given compound of Formula (I) is changed. The modification is most preferably an amino acid substitution. The modification is preferably a conservative substitution as defined herein.

In some embodiments the variant comprises Q147A/C/F/G/H/N/S/W/Y relative to SEQ ID NO: 2. Preferably, the modification Q147A/C/F/G/H/N/S/W/Y is Q147A/F/H/W. In some embodiments the variant comprises Q147A. In some embodiments the variant comprises Q147F. In some embodiments the variant comprises Q147H. In some embodiments the variant comprises Q147W.

In some embodiments the variant comprises N149D/E/N/P relative to SEQ ID NO: 2 or 3 (preferably 2). Preferably the modification N149D/E/N/P is N149D/E. In some embodiments the variant comprises N149D. In some embodiments the variant comprises N149E.

In some embodiments the variant comprises Q150I/Q/V relative to SEQ ID NO: 2 or 3 (preferably 2). In some embodiments the modification Q150I/Q/V is Q1501/V. In some embodiments the variant comprises Q150I. In some embodiments the variant comprises Q150V.

In some embodiments the variant comprises S154A/C/E/F/G/H/I/K/L/M/N/P/Q/R/S/T/V/Y relative to SEQ ID NO: 2 or 3 (preferably 2). In some embodiments the modification S154A/C/E/F/G/H/I/K/L/M/N/P/Q/R/S/T/V/Y is S154K/P/Q/A. In some embodiments the variant comprises S154K. In some embodiments the variant comprises S154P. In some embodiments the variant comprises S154Q. In some embodiments the variant comprises S154A.

In some embodiments the variant comprises A153F/H/R relative to SEQ ID NO: 2 or 3 (preferably 2). In some embodiment the variant comprises A153F. In some embodiments the variant comprises A153H. In some embodiments the variant comprises A153R.

In some embodiments the variant comprises F146L relative to SEQ ID NO: 2.

In some embodiments the variant comprises F155C relative to SEQ ID NO: 2.

The aforementioned modifications may be combined in some embodiments. Accordingly, the variant may comprise one or more of the modifications:
i) Q147A/C/F/G/H/N/S/W/Y relative to SEQ ID NO: 2, preferably Q147A/F/H/W; and/or
ii) N149D/E/N/P relative to SEQ ID NO: 2; preferably N149D/E; and/or
iii) Q150I/Q/V relative to SEQ ID NO: 2; preferably Q150I/V; and/or
iv) S154A/C/E/F/G/H/I/K/L/M/N/P/Q/R/S/T/V/Y relative to SEQ ID NO: 2; preferably S154K/P/Q/A; and/or
v) A153F/H/R relative to SEQ ID NO: 2; preferably A153F; and/or
vi) F146L relative to SEQ ID NO: 2; and/or
vii) F155C relative to SEQ ID NO: 2.

The variant may comprise:
Q147H/N149E/Q150I/S154P relative to SEQ ID NO: 2; or
Q147F/N149E/Q150V/S154P; or
Q147W/N149E/Q1501/S154K; or
Q147F/N149D/Q150I/S154P; or
Q147A/N149E/Q150V/S154K; or
Q147W/N149E/Q1501; or
Q147W/Q1501/S154Q; or
Q147W/N149D/Q150I/S154A.

The aforementioned modifications have been defined relative to SEQ ID NO: 2. As above, those skilled in the art will appreciate that the variant may comprise the corresponding modifications relative to SEQ ID NO: 3.

Most preferably, the polypeptide forms a zinc-finger structural motif.

The polypeptide may be at least 25 amino acids in length. The polypeptide is preferably, at least 30, 35, 40, 45, 50 or 55 amino acids in length. The polypeptide is most preferably at least 60 amino acids in length, preferably wherein the polypeptide comprises the amino acid sequence defined by positions 130-189 of SEQ ID NO: 2 except wherein the F145-F154 positions are defined according to the variant of the invention described herein.

The polypeptide may have at least about 50%, 60%, 70%, 80%, 90%, 95%, 96%, 97%, 98% or 99% sequence identity to SEQ ID NO: 2 or 3. The identity may be determined relative to the entire sequence of SEQ ID NOs 2 or 3. Or for example the identity may be determined over a sequence of 60 or more, 70 or more, 80 or more, 90 or more, 100 or more, 110 or more, 120 or more, 130 or more, 140 or more, or 150 or more contiguous amino acids comprised within SEQ ID NOs 2 and 3. Said contiguous amino acid sequence within SEQ ID NOs 2 and 3 preferably comprises the polypeptide defined according to SEQ ID NO: 1, and further preferably forms a zinc-finger structural motif.

The polypeptide is preferably capable of binding a compound of Formula (A), preferably a compound of Formula (I). More preferably, the binding of said polypeptide to the compound of Formula (A) or Formula (I) is stronger than the binding of the polypeptide defined according to SEQ ID NO: 2 or SEQ ID NO: 3 to said compound of Formula (A) or Formula (I).

Preferably, the binding of said polypeptide to the compound of Formula (A) or Formula (I) is at least about 1.25×, 1.5×, 2×, 3×, 4×, 5×, 10×, or 100× stronger than the binding of the polypeptide defined according to SEQ ID NO: 2 or SEQ ID NO: 3 to said compound of Formula (A) or Formula (I). Preferably, the relative affinities of the polypeptide defined according to the invention herein and the polypeptides according to SEQ ID Nos: 2 and 3 are quantified by the same assays. The binding assay used to quantify said the binding of said polypeptides to said compound of Formula (A) or Formula (I) may be any suitable protein-compound binding assays known in the art, wherein such assays may include computational in silico assays.

Additionally, or alternatively, the polypeptide may have a reduced ability to bind one or more of thalidomide, lenalidomide, pomalidomide, avadomide and iverdomide when compared to the polypeptide defined according to SEQ ID NO: 2 or SEQ ID NO: 3. More preferably, the polypeptide may have a reduced ability to bind all of thalidomide, lenalidomide, pomalidomide, avadomide and iverdomide when compared to the polypeptide defined according to SEQ ID NO: 2 or SEQ ID NO: 3. Preferably, the polypeptide may have at least about a 2.5%, 5%, 10%, 25%, or 50% reduced ability to bind one or more of thalidomide, lenalidomide, pomalidomide, avadomide and iverdomide when compared to the polypeptide defined according to SEQ ID NO: 2 or SEQ ID NO: 3. Preferably, the relative affinities of the polypeptide defined according to the invention herein and the polypeptides according to SEQ ID NOs: 2 and 3 are quantified by the same assays. The binding assay used to quantify said the binding of said polypeptides to said compound of Formula (A) or Formula (I) may be any suitable protein-compound binding assays known in the art, wherein such assays may include computational in silico assays.

The polypeptide may be attached to a target protein. The target protein may be any protein of interest. The target protein may particularly be a protein that is expressed in a cell.

The polypeptide may be attached to the target protein by any suitable means provided that the polypeptide does not, by virtue of said attachment alone, affect structure and/or function of the target protein. Preferably the polypeptide is attached to the target protein by genetic fusion and/or via a linker.

The polypeptide and target protein may be genetically fused. The polypeptide and target protein are genetically fused if both the polypeptide and target proteins are expressed in-frame from a single polynucleotide sequence.

Suitable linkers for attaching two proteins are well known in the art. The linker preferably covalently attaches the polypeptide and the target protein. The linker may directly attach the N-terminus of the polypeptide to the C-terminus of the target protein. The linker preferably directly attaches the C-terminus of the polypeptide to the N-terminus of the target protein.

Any suitable linker may be used. A linker may preferably be attached to the polypeptide and/or the target protein by genetic fusion. In some embodiments the linker and polypeptide are expressed as a single construct. In some embodiments the linker and polypeptide are expressed as a construct and the linker is then attached to the target polypeptide. In some embodiments the linker and target protein are expressed as a single construct. In some embodiments the linker and target protein are expressed as a construct and the linker is then attached to the polypeptide. In some embodiments the polypeptide, linker and target protein are expressed as a genetic construct. Practitioners are referred to methods disclosed in Sambrook et al., Molecular Cloning: A Laboratory Manual, 4th ed., Cold Spring Harbor Press, Plainsview, New York (2012).

The linker may be flexible or rigid. Preferred flexible peptide linkers are stretches of 2 to 40, such as 2 to 20, such as 4, 6, 8, 10 or 16, serine and/or glycine amino acids. More preferred flexible linkers include (SG)₁, (SG)₂, (SG)₃, (SG)₄, (SG)₅ and (SG)₈ wherein S is serine and G is glycine. A yet more preferred flexible linker is a poly-Glycine-Serine linker such as GGGGSGGGGS. Preferred rigid linkers are stretches of 2 to 30, such as 4, 6, 8, 16 or 24, proline amino acids. More preferred rigid linkers include (P)₁₂ wherein P is proline. A more preferred rigid linker is an amino acid sequence AEAAAKEAAAKEAAAKA. Another suitable rigid linker is an amino acid sequence.

The linker may be a chemical crosslinker. Examples of chemical crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate. The most preferred crosslinker is succinimidyl 3-(2-pyridyldithio)propionate (SPDP). Typically, the molecule is covalently attached to the bifunctional crosslinker before the molecule/crosslinker complex is covalently attached to the mutant monomer but it is also possible to covalently attach the bifunctional crosslinker to the monomer before the bifunctional crosslinker/monomer complex is attached to the molecule.

The linker is preferably resistant to dithiothreitol (DTT). Suitable linkers include, but are not limited to, iodoacetamide-based and Maleimide-based linkers.

The linker may be or comprise a polypeptide-based linker and may comprise one or more terminal natural or non-natural amino acids having a reactive side chain capable of binding to a reactive functional group on the polypeptide and/or the target protein. The or each terminal group may independently for example be a cysteine or a lysine residue.

The attachment between the linker and polypeptide/target protein is not limited and any suitable attachment chemistry can be used. Many suitable reactive groups and their chemical targets are known in the art. Some exemplary reactive groups and their corresponding targets include aryl azides which may react with amine, carbodiimides which may react with amines and carboxyl groups, hydrazides which may react with carbohydrates, hydroxmethyl phosphines which may react with amines, imidoesters which may react with amines, isocyanates which may react with hydroxyl groups, carbonyls which may react with hydrazines, maleimides which may react with sulfhydryl groups, NHS-esters which may react with amines, PFP-esters which may react with amines, psoralens which may react with thymine, pyridyl disulfides which may react with sulfhydryl groups, vinyl sulfones which may react with sulfhydryl amines and hydroxyl groups, vinylsulfonamides, and the like.

Other suitable chemistry for conjugating the polypeptide to the polynucleotide includes click chemistry. Many suitable click chemistry reagents are known in the art. Suitable examples of click chemistry include, but are not limited to, the following:
(a) copper(I)-catalyzed azide-alkyne cycloadditions (azide alkyne Huisgen cycloadditions);
(b) strain-promoted azide-alkyne cycloadditions; including alkene and azide [3+2]cycloadditions; alkene and tetrazine inverse-demand Diels-Alder reactions; and alkene and tetrazole photoclick reactions;
(c) copper-free variant of the 1,3 dipolar cycloaddition reaction, where an azide reacts with an alkyne under strain, for example in a cyclooctane ring such as in bicycle[6.1.0]nonyne (BCN);
(d) the reaction of an oxygen nucleophile on one linker with an epoxide or aziridine reactive moiety on the other; and
(e) the Staudinger ligation, where the alkyne moiety can be replaced by an aryl phosphine, resulting in a specific reaction with the azide to give an amide bond.

The linker may comprise a naturally occurring reactive functional group which can be used to facilitate conjugation to the polypeptide/target protein. For example, a cysteine residue can be used to form a disulphide bond to a cysteine residue on the polypeptide/target protein. A cysteine residue can be used to react with a mameimide group. For instance, a polypeptide/target protein as described herein can conjugated to a linker having a C-terminal cysteine residue using MBS (m-maleimidobenzoyl-N-hydroxysuccinimide ester). MBS is an amine-to-sulfhydryl crosslinker and thus can attach the cysteine residue of the construct to a lysine residue of the polypeptide/target protein. Other cysteine/lysine reactive groups can be used.

As discussed above, the system provided herein comprises a compound of Formula (A):

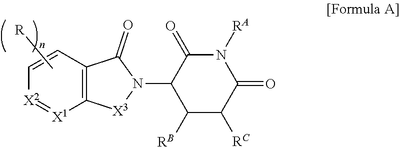

wherein
   X¹ is C—R and X² is C—R or N; or X¹ is C—R or N and X² is C—R;
   X³ is CHR^{D} or C═O;
   each R, which may be the same or different, is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, 5- to 10-membered (e.g. C₅₋₁₀) heteroaryl, 5- to 10-membered (e.g. C₅₋₁₀) heterocyclyl, C₅₋₁₀ carbocyclyl, OR²⁰, N(R²⁰)₂ and halogen;
      and wherein each aryl, heteroaryl, heterocyclyl and carbocyclyl group is independently unsubstituted or is substituted with 1, 2 or 3, preferably 1 or 2 substituents independently selected from R²⁰, OR²⁰, N(R²⁰)₂ and halogen;
   each R¹⁰, which may be the same or different, is independently H or C₁₋₄ alkyl which is unsubstituted or substituted with 1, 2 or 3 substituents independently selected from OR²⁰, N(R²⁰)₂ and halogen;
   n is an integer from 0 to 2;
   R^{A} is H or C₁₋₄ alkyl which is unsubstituted or is substituted with 1, 2 or 3 substituents independently selected from OR²⁰, N(R²⁰)₂ and halogen;
   R^{B} is H or C₁₋₄ alkyl which is unsubstituted or is substituted with 1, 2 or 3 substituents independently selected from OR²⁰, N(R²⁰)₂ and halogen;
   R^{C} is H or C₁₋₄ alkyl which is unsubstituted or is substituted with 1, 2 or 3 substituents independently selected from OR²⁰, N(R²⁰)₂ and halogen;
   R^{D} is H or C₁₋₄ alkyl which is unsubstituted or is substituted with 1, 2 or 3 substituents independently selected from OR²⁰, N(R²⁰)₂ and halogen; and
   each R²⁰, which may be the same or different is independently H or C₁₋₆ alkyl which is independently unsubstituted or is substituted with 1, 2 or 3 substituents independently selected from OR³⁰, N(R³⁰)₂ and halogen, wherein R³⁰ is H or methyl; preferably each R²⁰ is independently H or methyl.
      In some embodiments, the system provided herein comprises a compound of Formula (A) as described above, wherein:
each R, which may be the same or different, is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, C₅₋₁₀ heteroaryl, C₅₋₁₀ heterocyclyl, and C₅₋₁₀ carbocyclyl;
   wherein each alkyl, alkenyl and alkynyl group is independently unsubstituted or is substituted with 1, 2 or 3 substituents independently selected from C₆₋₁₀ aryl, C₅₋₁₀ heteroaryl, C₅₋₁₀ heterocyclyl, C₅₋₁₀ carbocyclyl, OR²⁰, N(R²⁰)₂ and halogen;
   and wherein each aryl, heteroaryl, heterocyclyl and carbocyclyl group is independently unsubstituted or is substituted with 1 or 2 substituents independently selected from R²⁰, OR²⁰, N(R²⁰)₂ and halogen; and
each R²⁰, which may be the same or different is independently H or methyl.
   Typically, the system provided herein comprises a compound of Formula (I):

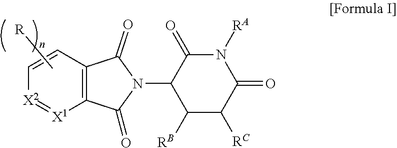

wherein
   X¹ is C—R and X² is C—R or N; or X¹ is C—R or N and X² is C—R;
   each R, which may be the same or different, is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, 5- to 10-membered (e.g. C₅₋₁₀) heteroaryl, 5- to 10-membered (e.g. C₅₋₁₀) heterocyclyl, C₅₋₁₀ carbocyclyl, OR²⁰, N(R²⁰)₂ and halogen;
      and wherein each aryl, heteroaryl, heterocyclyl and carbocyclyl group is independently unsubstituted or is substituted with 1, 2 or 3, preferably 1 or 2 substituents independently selected from R²⁰, OR²⁰, N(R²⁰)₂ and halogen;
   each R¹⁰, which may be the same or different, is independently H or C₁₋₄ alkyl which is unsubstituted or substituted with 1, 2 or 3 substituents independently selected from OR²⁰, N(R²⁰)₂ and halogen;
   n is an integer from 0 to 2;
   R^{A} is H or C₁₋₄ alkyl which is unsubstituted or is substituted with 1, 2 or 3 substituents independently selected from OR²⁰, N(R²⁰)₂ and halogen;
   R^{B} is H or C₁₋₄ alkyl which is unsubstituted or is substituted with 1, 2 or 3 substituents independently selected from OR²⁰, N(R²⁰)₂ and halogen;
   R^{C} is H or C₁₋₄ alkyl which is unsubstituted or is substituted with 1, 2 or 3 substituents independently selected from OR²⁰, N(R²⁰)₂ and halogen; and
   each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl; preferably each R²⁰ is independently H or methyl.

In some embodiments, the system provided herein comprises a compound of Formula (I) as described above, wherein:
each R, which may be the same or different, is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, C₅₋₁₀ heteroaryl, C₅₋₁₀ heterocyclyl, and C₅₋₁₀ carbocyclyl;
   wherein each alkyl, alkenyl and alkynyl group is independently unsubstituted or is substituted with 1, 2 or 3 substituents independently selected from C₆₋₁₀ aryl, C₅₋₁₀ heteroaryl, C₅₋₁₀ heterocyclyl, C₅₋₁₀ carbocyclyl, OR²⁰, N(R²⁰)₂ and halogen;
   and wherein each aryl, heteroaryl, heterocyclyl and carbocyclyl group is independently unsubstituted or is substituted with 1 or 2 substituents independently selected from R²⁰, OR²⁰, N(R²⁰)₂ and halogen; and
each R²⁰, which may be the same or different, is independently H or methyl.

Typically, X¹ is C—R and X² is C—R. Typically, when X¹ is C—R and X² is C—R, each R is different. In some embodiments, X¹ is CR¹ where R¹ is as defined herein. In some embodiments, X² is CR² where R² is as defined herein. Typically, R^{A} is H or C₁₋₃ alkyl, more typically H or C₁₋₂ alkyl, more typically H or C₁ alkyl, most typically H. When R^{A} is an alkyl group, the alkyl group is typically unsubstituted or is substituted with 1 or 2 substituents independently selected from OR²⁰, N(R²⁰)₂ and halogen, more often by OH, NH₂ or halogen. More often, when R^{A} is an alkyl group, the alkyl group is unsubstituted or is substituted with 1 OH or NH₂ group.

Typically, R^{B} is H or C₁₋₃ alkyl, more typically H or C₁₋₂ alkyl, more typically H or C₁ alkyl, most typically H. When R^{B} is an alkyl group, the alkyl group is typically unsubstituted or is substituted with 1 or 2 substituents independently selected from OR²⁰, N(R²⁰)₂ and halogen, more often by OH, NH₂ or halogen. More often, when R^{B} is an alkyl group, the alkyl group is unsubstituted or is substituted with 1 OH or NH₂ group.

Typically, R^{C} is H or C₁₋₃ alkyl, more typically H or C₁₋₂ alkyl, more typically H or C₁ alkyl, most typically H. When R^{C} is an alkyl group, the alkyl group is typically unsubstituted or is substituted with 1 or 2 substituents independently selected from OR²⁰, N(R²⁰)₂ and halogen, more often by OH, NH₂ or halogen. More often, when R^{C} is an alkyl group, the alkyl group is unsubstituted or is substituted with 1 OH or NH₂ group.

Typically, R^{D} is H or C₁₋₃ alkyl, more typically H or C₁₋₂ alkyl, more typically H or C₁ alkyl, most typically H. When R^{D} is an alkyl group, the alkyl group is typically unsubstituted or is substituted with 1 or 2 substituents independently selected from OR²⁰, N(R²⁰)₂ and halogen, more often by OH, NH₂ or halogen. More often, when R^{D} is an alkyl group, the alkyl group is unsubstituted or is substituted with 1 OH or NH₂ group.

Thus, typically, X³ is CH₂ or C═O. For instance, X³ may be CH₂. X³ may be C═O.

Any of the compounds of Formula (A), (I), (Ia) or (Ib), or (Ic) or (Id) as described herein in more detail may be comprised in the system provided herein.

In some embodiments the compound of Formula (I) may be a compound of formula (Ia):

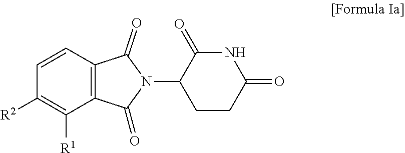

wherein
   R¹ is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, 5- to 10-membered (e.g. C₅₋₁₀) heteroaryl, 5- to 10-membered (e.g. C₅₋₁₀) heterocyclyl, C₅₋₁₀ carbocyclyl, OR²⁰, N(R²⁰)₂ and halogen;
      and wherein each aryl, heteroaryl, heterocyclyl and carbocyclyl group is independently unsubstituted or is substituted with 1, 2 or 3, preferably 1 or 2 substituents independently selected from R²⁰, OR²⁰, N(R²⁰)₂ and halogen;
   R² is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, 5- to 10-membered (e.g. C₅₋₁₀) heteroaryl, 5- to 10-membered (e.g. C₅₋₁₀) heterocyclyl, C₅₋₁₀ carbocyclyl, OR²⁰, N(R²⁰)₂ and halogen;
      and wherein each aryl, heteroaryl, heterocyclyl and carbocyclyl group is independently unsubstituted or is substituted with 1, 2 or 3, preferably 1 or 2 substituents independently selected from R²⁰, OR²⁰, N(R²⁰)₂ and halogen;
   each R¹⁰, which may be the same or different, is independently H or methyl; and
   each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl; preferably each R²⁰ is independently H or methyl.
      In some embodiments, the system provided herein comprises a compound of Formula (Ia) as described above, wherein:
R¹ is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, C₅₋₁₀ heteroaryl, C₅₋₁₀ heterocyclyl, and C₅₋₁₀ carbocyclyl;
   wherein each alkyl, alkenyl and alkynyl group is independently unsubstituted or is substituted with 1, 2 or 3 substituents independently selected from C₆₋₁₀ aryl, C₅₋₁₀ heteroaryl, C₅₋₁₀ heterocyclyl, C₅₋₁₀ carbocyclyl, OR²⁰, N(R²⁰)₂ and halogen;
   and wherein each aryl, heteroaryl, heterocyclyl and carbocyclyl group is independently unsubstituted or is substituted with 1 or 2 substituents independently selected from R²⁰, OR²⁰, N(R²⁰)₂ and halogen;
R² is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, C₅₋₁₀ heteroaryl, C₅₋₁₀ heterocyclyl, and C₅₋₁₀ carbocyclyl;
   wherein each alkyl, alkenyl and alkynyl group is independently unsubstituted or is substituted with 1, 2 or 3 substituents independently selected from C₆₋₁₀ aryl, C₅₋₁₀ heteroaryl, C₅₋₁₀ heterocyclyl, C₅₋₁₀ carbocyclyl, OR²⁰, N(R²⁰)₂ and halogen;
   and wherein each aryl, heteroaryl, heterocyclyl and carbocyclyl group is independently unsubstituted or is substituted with 1 or 2 substituents independently selected from R²⁰, OR²⁰, N(R²⁰)₂ and halogen;
each R¹⁰, which may be the same or different, is independently H or methyl; and
each R²⁰, which may be the same or different, is independently H or methyl.

Typically, R¹ may be selected from hydrogen, N(R¹⁰)₂, OR¹⁰, halogen, C₁₋₄ alkyl, C₂₋₄ alkynyl, O—C₁₋₄ alkyl, O—C₂₋₄ alkynyl, NR¹⁰—C₁₋₄ alkyl, NR¹⁰—C₂₋₄ alkynyl, C₆ aryl, and 5- to 6-membered (e.g. C₅₋₆) heterocyclyl, and 5- to 6-membered (e.g. C₅0.6) heteroaryl. More typically, R¹ is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅0.6) heterocyclyl. Still more typically, R¹ is selected from H, NH₂, OH, OMe, halogen, methyl, NH—C₁₋₃ alkyl, and unsubstituted NH—C₃ alkynyl.

Typically, when R¹ is or comprises an alkyl or alkynyl group, said alkyl or alkynyl group is independently unsubstituted or is substituted with 1, 2 or 3 substituents independently selected from C₆ aryl, 5- to 6-membered (e.g. C₅0.6) heteroaryl, and halogen. More typically, said alkyl or alkynyl group is independently unsubstituted or is substituted with 1 or 2 substituents independently selected from C₆ aryl, 5- to 6-membered (e.g. C₅0.6) heteroaryl or with 1-3 halogen substituents. Still more typically, said alkyl or alkynyl group is independently unsubstituted or is substituted with 1 or 2 substituents independently selected from C₆ aryl, 5- to 6-membered (e.g. C₅₋₆) heteroaryl or with 1-3 halogen substituents.

When R¹ comprises an aryl, heteroaryl, heterocyclyl or carbocyclyl group, each said group is typically independently unsubstituted or is substituted with 1, 2 or 3 R²⁰ groups, preferably one R²⁰ group. Often said group is unsubstituted. Often said group is substituted by methyl.

Typically, R² may be selected from hydrogen, N(R¹⁰)₂, OR¹⁰, halogen, C₁₋₄ alkyl, C₂₋₄ alkynyl, O—C₁₋₄ alkyl, O—C₂₋₄ alkynyl, NR¹⁰—C₁₋₄ alkyl, NR¹⁰—C₂₋₄ alkynyl, C₆ aryl, and 5- to 6-membered (e.g. C₅₋₆) heterocyclyl, and 5- to 6-membered (e.g. C₅₋₆) heteroaryl. More typically, R² is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl. Still more typically, R² is selected from H, N(R¹⁰)₂, OH, OMe, halogen, unsubstituted C₁₋₄ alkyl, CF₃, phenyl, piperazinyl, and morpholinyl.

Typically, when R² is or comprises an alkyl or alkynyl group, said alkyl or alkynyl group is independently unsubstituted or is substituted with 1, 2 or 3 substituents independently selected from C₆ aryl, 5- to 6-membered (e.g. C₅₋₆) heteroaryl, and halogen. More typically, said alkyl or alkynyl group is independently unsubstituted or is substituted with 1 or 2 substituents independently selected from C₆ aryl, 5- to 6-membered (e.g. C₅₋₆) heteroaryl or with 1-3 halogen substituents. Still more typically, said alkyl or alkynyl group is independently unsubstituted or is substituted with 1 or 2 substituents independently selected from C₆ aryl, 5- to 6-membered (e.g. C₅₋₆) heteroaryl or with 1-3 halogen substituents.

When R² comprises an aryl, heteroaryl, heterocyclyl or carbocyclyl group, each said group is typically independently unsubstituted or is substituted with 1, 2 or 3 R²⁰ groups, preferably one R²⁰ group. Often said group is unsubstituted. Often said group is substituted by methyl. Sometimes said group is substituted by C₁-C₄ alkyl such as methyl, ethyl, propyl (e.g. ⁱPr), butyl (e.g. ^{t}Bu). Sometimes the group is substituted by 1, 2 or 3 C₁-C₄ alkyl groups such as e.g. 1, 2 or 3 methyl groups.

Typically:
i) R¹ is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl;
ii) R² is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl;
wherein each alkyl and alkynyl group is independently unsubstituted or is substituted with 1 or 2 substituents independently selected from C₆ aryl, 5- to 6-membered (e.g. C₅₋₆) heteroaryl or with 1-3 halogen substituents;
and wherein each aryl, heteroaryl, and heterocyclyl group is independently unsubstituted or is substituted with 1, 2 or 3 R²⁰ groups, preferably one R²⁰ group.

More typically:
i) R¹ is selected from H, NH₂, OH, OMe, halogen, methyl, NH—C₁₋₃ alkyl, and unsubstituted NH—C₃ alkynyl; wherein each C₁₋₃ alkyl group is independently unsubstituted or is substituted with 1 or 2 phenyl or pyridinyl groups; wherein said phenyl or pyridinyl groups are each independently unsubstituted or substituted by methyl; and
ii) R² is selected from H, N(R¹⁰)₂, OH, OMe, halogen, unsubstituted C₁₋₄ alkyl, CF₃, phenyl, piperazinyl, and morpholinyl; wherein said phenyl, piperazinyl, and morpholinyl groups are independently unsubstituted or substituted by C₁₋₆ alkyl, preferably C₁₋₄ alkyl, more preferably methyl.

Often, R¹⁰ is H. Often, R¹⁰ is methyl.

Often, each R²⁰ is independently H or C₁₋₆ alkyl which is independently unsubstituted or is substituted with OH, NH₂, or with 1, 2 or 3 halogen substituents. More often, each R²⁰ is independently H or unsubstituted C₁₋₆ alkyl. More often, each R²⁰ is independently H or unsubstituted C₁₋₄ alkyl. More often, each R²⁰ is independently H or unsubstituted C₁₋₃ alkyl. More often, each R²⁰ is independently H or unsubstituted C₁₋₂ alkyl. Preferably, R²⁰ is H or methyl. Often, R²⁰ is H. Often, R²⁰ is methyl.

In another aspect, the system provided herein comprises a compound of Formula (Ic):

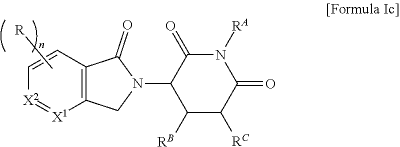

wherein X¹, X², R, n, R^{A}, R^{B}, and R^{C} are as defined above; for example, in some preferred aspects X¹, X², R, n, R^{A}, R^{B}, and R^{C} are as defined for Formula (I).

In another aspect, the system provided herein comprises a compound of Formula (Id):

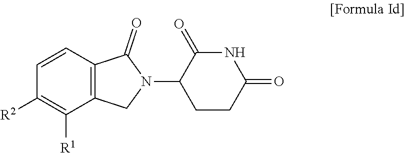

wherein R¹ and R² are as defined above; for example, in some preferred aspects R¹ and R² are as defined for Formula (Ia).

For example, in some embodiments the compound is a compound of Formula (Id), and
i) R¹ is selected from H, N(R¹⁰)₂, OR¹⁰, halogen, C₁₋₄ alkyl, NR¹⁰-C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl;
ii) R² is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl;
wherein each alkyl and alkynyl group is independently unsubstituted or is substituted with 1 or 2 substituents independently selected from C₆ aryl, 5- to 6-membered (e.g. C₅₋₆) heteroaryl or with 1-3 halogen substituents;
and wherein each aryl, heteroaryl, and heterocyclyl group is independently unsubstituted or is substituted with 1, 2 or 3 R²⁰ groups, preferably one R²⁰ group.

In some preferred aspects, the compound is a compound of formula (I) or formula (Ic) (preferably formula (I)) and X¹ is CR¹ and X² is CR²; or the compound is a compound of formula (Ia) or (Id) (preferably formula (Ia)); and R¹ and R² are according to any of the following. More preferably the compound is a compound of formula (I) and X¹ is CR¹ and X² is CR²; or the compound is a compound of formula (Ia); and R¹ and R² are according to any of the following (preferably R¹ and R² are according to any of Compounds 1 to 36 in the following table):

| Compound | | |
|---|---|---|
| number | R¹ | R² |
| 1 | 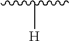 [SMILES: *[H]] | 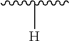 [SMILES: *[H]] |
| 2 | 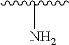 [SMILES: *N] | 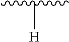 [SMILES: *[H]] |
| 3 | 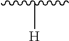 [SMILES: *[H]] | 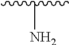 [SMILES: *N] |
| 4 | 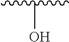 [SMILES: *O] | 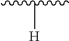 [SMILES: *[H]] |
| 5 | 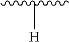 [SMILES: *[H]] | 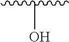 [SMILES: *O] |
| 6 | 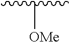 [SMILES: *OC] | 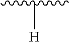 [SMILES: *[H]] |
| 7 | 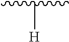 [SMILES: *[H]] | 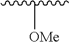 [SMILES: *OC] |
| 8 | 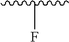 [SMILES: *F] | 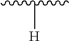 [SMILES: *[H]] |
| 9 | 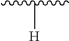 [SMILES: *[H]] | 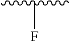 [SMILES: *F] |
| 10 | 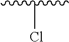 [SMILES: *Cl] | 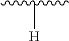 [SMILES: *[H]] |
| 11 | 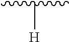 [SMILES: *[H]] | 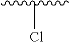 [SMILES: *Cl] |
| 12 | 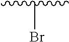 [SMILES: *Br] | 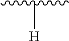 [SMILES: *[H]] |
| 13 | 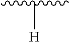 [SMILES: *[H]] | 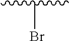 [SMILES: *Br] |
| 14 | 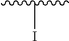 [SMILES: *I] | 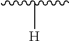 [SMILES: *[H]] |
| 15 | 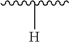 [SMILES: *[H]] | 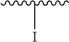 [SMILES: *I] |
| 16 | 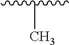 | 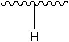 [SMILES: *[H]] |
| 17 | 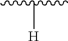 [SMILES: *[H]] | 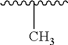 |
| 18 | 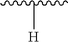 [SMILES: *[H]] | 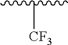 [SMILES: *C(F)(F)F] |
| 19 | 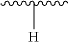 [SMILES: *[H]] | 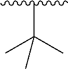 [SMILES: *C(C)(C)C] |
| 20 | 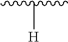 [SMILES: *[H]] | 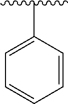 [SMILES: *c1ccccc1] |
| 21 | 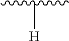 [SMILES: *[H]] | 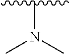 |
| 22 | 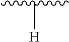 [SMILES: *[H]] | 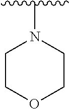 [SMILES: *N1CCOCC1] |
| 23 | 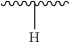 [SMILES: *[H]] | 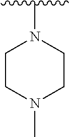 [SMILES: *N1CCN(C)CC1] |
| 24 | 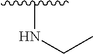 [SMILES: *NCC] | 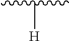 [SMILES: *[H]] |
| 25 | 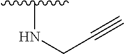 [SMILES: *NCC#C] | 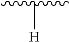 [SMILES: *[H]] |
| 26 | 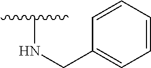 [SMILES: *NCc1ccccc1] | 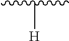 [SMILES: *[H]] |
| 27 | 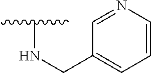 [SMILES: *NCc1cccnc1] | 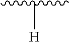 [SMILES: *[H]] |
| 28 | 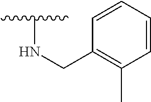 [SMILES: *NCc1ccccc1C] | 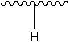 [SMILES: *[H]] |
| 29 | 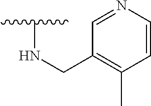 [SMILES: *NCc1cnccc1C] | 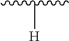 [SMILES: *[H]] |
| 30 | 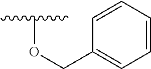 [SMILES: *OCc1ccccc1] | 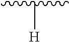 [SMILES: *[H]] |
| 31 | 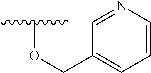 [SMILES: *OCc1cccnc1] | 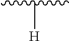 [SMILES: *[H]] |
| 32 | 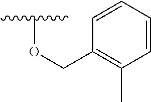 [SMILES: *OCc1ccccc1C] | 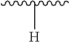 [SMILES: *[H]] |
| 33 | 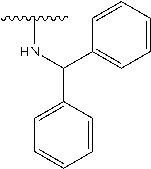 [SMILES: *NC(c1ccccc1)c1ccccc1] | 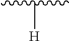 [SMILES: *[H]] |
| 34 | 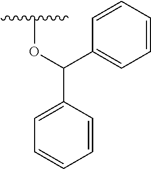 [SMILES: *OC(c1ccccc1)c1ccccc1] | 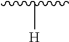 [SMILES: *[H]] |
| 35 | 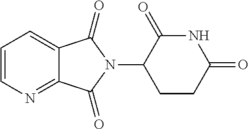 | |
| 36 | 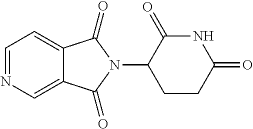 | |
| 37 | 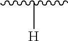 [SMILES: *[H]] | 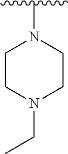 [SMILES: *N1CCN(CC)CC1] |
| 38 | 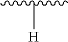 [SMILES: *[H]] | 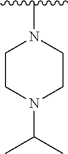 [SMILES: *N1CCN(C(C)C)CC1] |
| 39 | 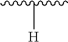 [SMILES: *[H]] | 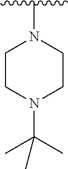 [SMILES: *N1CCN(C(C)(C)C)CC1] |
| 40 | 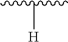 [SMILES: *[H]] | 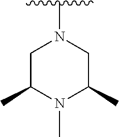 |

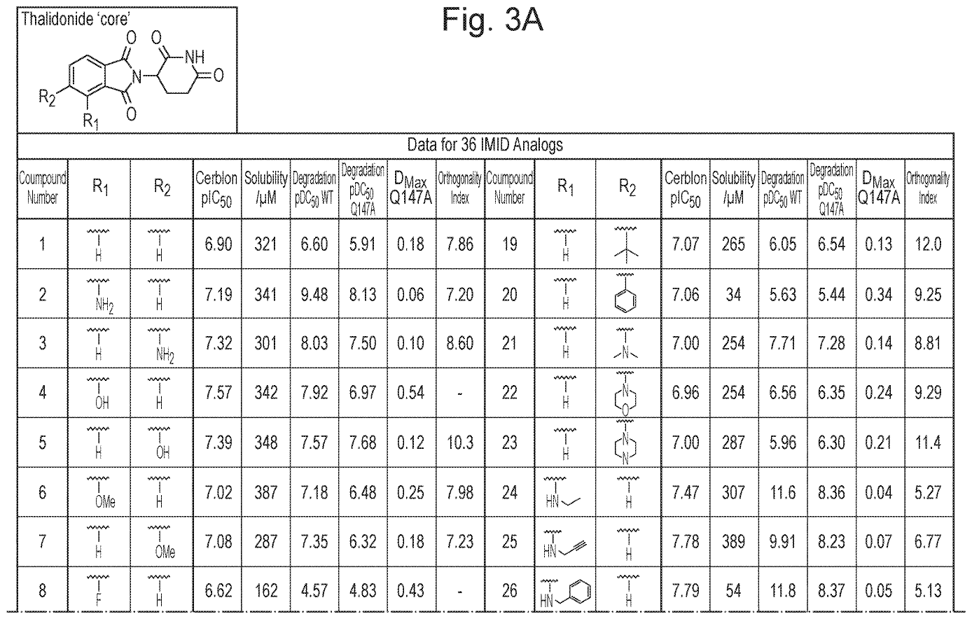

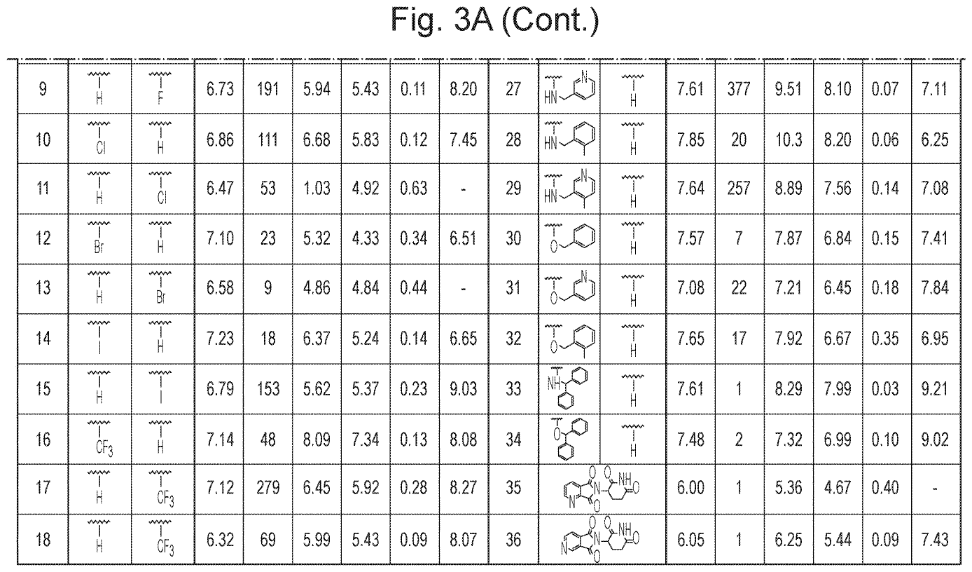

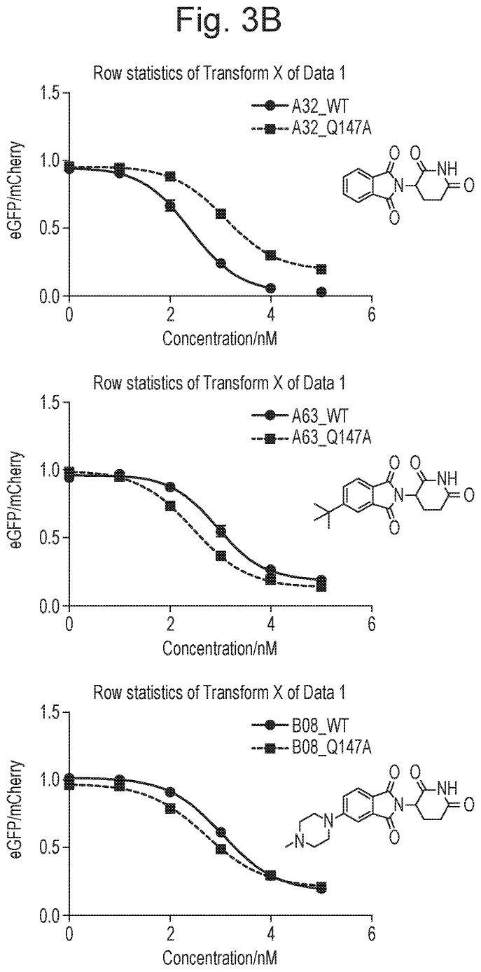

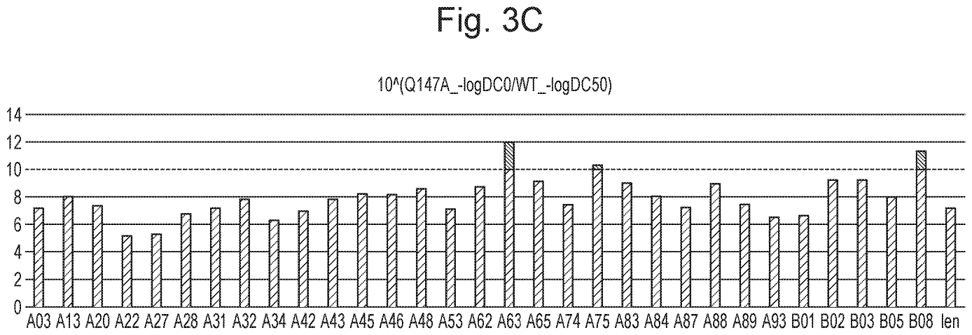

The system according to the invention may preferably comprise:
i) a polypeptide that is at least 60 amino acids in length, wherein the polypeptide has at least 70% sequence identity to region of the same length within SEQ ID NO: 2, or more preferably at least 90% sequence identity, and preferably wherein the polypeptide forms a zinc finger structural motif, and wherein the polypeptide comprises the amino acids at positions 146, 147, 149, 150, 152, 153, 154, 155 and 167 corresponding to SEQ ID NO: 2 the amino acids set out in column 4 of Table 1; and preferably
ii) a compound of Formula I wherein X¹ is CR¹, X² is CR²; preferably wherein the compound is a compound of Formula (Ta); and wherein R¹ is H and R² is H. A preferred example of such a compound is A32 (depicted in FIG. 3).

The system according to the invention may preferably comprise:
i) a polypeptide that is at least 60 amino acids in length, wherein the polypeptide has at least 70% sequence identity to region of the same length within SEQ ID NO: 2, or more preferably at least 90% sequence identity, and preferably wherein the polypeptide forms a zinc finger structural motif, and wherein the polypeptide comprises the amino acids at positions 146, 147, 149, 150, 152, 153, 154, 155 and 167 corresponding to SEQ ID NO: 2 the amino acids set out in column 4 of Table 2; and preferably
ii) a compound of Formula I wherein X¹ is CR¹, X² is CR²; preferably wherein the compound is a compound of Formula (Ta); and wherein R¹ is H and R² is C₁₋₄ alkyl; more preferably ^{t}Bu. A preferred example of such a compound is A63 (depicted in FIG. 3).

The system according to the invention may preferably comprise:
i) a polypeptide that is at least 60 amino acids in length, wherein the polypeptide has at least 70% sequence identity to region of the same length within SEQ ID NO: 2, or more preferably at least 90% sequence identity, and preferably wherein the polypeptide forms a zinc finger structural motif, and wherein the polypeptide comprises the amino acids at positions 146, 147, 149, 150, 152, 153, 154, 155 and 167 corresponding to SEQ ID NO: 2 the amino acids set out in column 4 of Table 3; and preferably
ii) a compound of Formula I wherein X¹ is CR¹, X² is CR²; preferably wherein the compound is a compound of Formula (Ta); and wherein R¹ is H and R² is 6-membered (e.g. C₆) heterocyclyl; more preferably N-Me-piperazine. A preferred example of such a compound is B08 (depicted in FIG. 3).

The system according to the invention may preferably comprise:
i) a polypeptide that is at least 60 amino acids in length, wherein the polypeptide has at least 70% sequence identity to region of the same length within SEQ ID NO: 2, or more preferably at least 90% sequence identity, and preferably wherein the polypeptide forms a zinc finger structural motif, and wherein the polypeptide comprises the amino acids at positions 146, 147, 149, 150, 152, 153, 154, 155 and 167 corresponding to SEQ ID NO: 2 the amino acids set out in column 4 of Table 4; and preferably
ii) a compound of Formula I wherein X¹ is CR¹, X² is CR²; preferably wherein the compound is a compound of Formula (Ta); and wherein R¹ is NH—C₁₋₃ alkyl; more preferably —NH—CH(Ph)₂; and R² is H. A preferred example of such a compound is A65.

The system according to the invention may preferably comprise:
i) a polypeptide that is at least 60 amino acids in length, wherein the polypeptide has at least 70% sequence identity to region of the same length within SEQ ID NO: 2, or more preferably at least 90% sequence identity, and preferably wherein the polypeptide forms a zinc finger structural motif, and wherein the polypeptide comprises the amino acids at positions 146, 147, 149, 150, 152, 153, 154, 155 and 167 corresponding to SEQ ID NO: 2 the amino acids set out in column 4 of Table 5; and preferably
ii) a compound of Formula I wherein X¹ is CR¹, X² is CR²; preferably wherein the compound is a compound of Formula (Ta); and wherein R¹ is H and R² is 6-membered (e.g. C₆) heterocyclyl; more preferably morpholine. A preferred example of such a compound B03.

The system according to the invention may preferably comprise:
i) a polypeptide that is at least 60 amino acids in length, wherein the polypeptide has at least 70% sequence identity to region of the same length within SEQ ID NO: 2, or more preferably at least 90% sequence identity, and preferably wherein the polypeptide forms a zinc finger structural motif, and wherein the polypeptide comprises the amino acids at positions 146, 147, 149, 150, 152, 153, 154, 155 and 167 corresponding to SEQ ID NO: 2 the amino acids set out in column 4 of Table 5; and preferably
ii) a compound of Formula I wherein X¹ is CR¹, X² is CR²; preferably wherein the compound is a compound of Formula (Ta); and wherein R¹ is H and R² is 6-membered (e.g. C₆) heterocyclyl; more preferably piperazine which is substituted with 1, 2 or 3 unsubstituted C₁₋₆ alkyl groups. A preferred example of such a compound wherein R² is piperazine which is substituted with one C₂ alkyl group is B22. A preferred example of such a compound wherein R² is piperazine which is substituted with one C₃ alkyl group is B25. A preferred example of such a compound wherein R² is piperazine which is substituted with one C₄ alkyl group is B31. A preferred example of such a compound wherein R² is piperazine which is substituted with three C₁ alkyl (methyl) groups is B32.

The system according to the invention may preferably comprise:
i) a polypeptide that is at least 60 amino acids in length, wherein the polypeptide has at least 70% sequence identity to region of the same length within SEQ ID NO: 2, or more preferably at least 90% sequence identity, and preferably wherein the polypeptide forms a zinc finger structural motif, and wherein the polypeptide comprises the amino acids at positions 146, 147, 149, 150, 152, 153, 154, 155 and 167 corresponding to SEQ ID NO: 2 the amino acids set out in column 4 of Table 5; and preferably
ii) a compound of Formula A wherein R^{A} is H, R^{B} is H, R^{C} is H, X³ is CHR^{D}, R^{D}is H, X¹ is CR¹, X² is CR²; preferably wherein the compound is a compound of Formula (Id); and wherein R¹ is H and R² is 6-membered (e.g. C₆) heterocyclyl; more preferably piperazine which is substituted with 1, 2 or 3 unsubstituted C₁₋₆ alkyl groups. A preferred example of such a compound wherein R² is piperazine which is substituted with one C₁ alkyl group is C01.

TABLE 1
A32 orthogonality; Estimated LogFC Coefficient calculated from the limma voom linear regression contrast of the positive
vs negative read counts for each individual mutant degron as defined relative to SEQ ID NO: 2.

| | | | Annotation of amino acids according | |
|---|---|---|---|---|
| | | | to their position in | |
| | | | SEQ ID NO: 2, wherein the stated one | |
| | | | letter amino acid may be the original | |
| | | | amino acid found in SEQ ID NO: 2 | |
| | | DNA sequence encoding positions F146-L167 of SEQ ID NO: 2 absent any | or a substitution (first numerical | |
| SEQ ID | | substitutions defined according to the encoding sequence below and | digit dropped, i.e. position 146 in | |
| NO | Degron-ID | the variant amino acid sequence set out in column 4 of this Table | SEQ ID NO: 2 = 46 as stated below) | A32_logFC |
| 4 | 3108 | TTCCAGTGTCCCGTATGTGGGGCAAAGTTTactcagaaaggtaacctcctccgccacattaaaCTG | 46F_47Q_49P_50V_52G_53A_54K_55F_67L_ | 3.961842815 |
| 5 | 3112 | TTCCAATGTCCCGTTTGTGGGGCACCTTTTactcagaaaggtaacctcctccgccacattaaaCTG | 46F_47Q_49P_50V_52G_53A_54P_55F_67L_ | 3.871679632 |
| 6 | 3016 | TTCCAGTGTAATATCTGTGGGGCAACATTTactcagaaaggtaacctcctccgccacattaaaCTG | 46F_47Q_49N_50I_52G_53A_54T_55F_67L_ | 3.866649605 |
| 7 | 3007 | TTCCAGTGTAACATATGTGGGGCAATCTTTactcagaaaggtaacctcctccgccacattaaaCTG | 46F_47Q_49N_50I_52G_53A_54I_55F_67L_ | 3.811402414 |
| 8 | 3062 | TTCCAGTGTCCCATCTGTGGGGCAGACTTTactcagaaaggtaacctcctccgccacattaaaCTG | 46F_47Q_49P_50I_52G_53A_54D_55F_67L_ | 3.805887286 |
| 9 | 3116 | TTCCAGTGTCCTGTATGTGGGGCAACTTTTactcagaaaggtaacctcctccgccacattaaaCTG | 46F_47Q_49P_50V_52G_53A_54T_55F_67L_ | 3.794141739 |
| 10 | 3067 | TTCCAGTGTCCTATCTGTGGGGCAATTTTTactcagaaaggtaacctcctccgccacattaaaCTG | 46F_47Q_49P_50I_52G_53A_54I_55F_67L_ | 3.792360734 |
| 11 | 3075 | TTCCAGTGTCCTATCTGTGGGGCAAGCTTTactcagaaaggtaacctcctccgccacattaaaCTG | 46F_47Q_49P_50I_52G_53A_54S_55F_67L_ | 3.789972901 |
| 12 | 2993 | TTCCAGTGTGAGGTTTGTGGGGCACAGTTTactcagaaaggtaacctcctccgccacattaaaCTG | 46F_47Q_49E_50V_52G_53A_54Q_55F_67L_ | 3.771940972 |
| 13 | 3061 | TTCCAGTGTCCTATCTGTGGGGCATGTTTTactcagaaaggtaacctcctccgccacattaaaCTG | 46F_47Q_49P_50I_52G_53A_54C_55F_67L_ | 3.771072345 |
| 14 | 3103 | TTCCAGTGTCCCGTATGTGGGGCAGAGTTTactcagaaaggtaacctcctccgccacattaaaCTG | 46F_47Q_49P_50V_52G_53A_54E_55F_67L_ | 3.761673523 |
| 15 | 6717 | CTGCAGTGCCCTATCTGCGGCTTCGTTTGTactcagaaaggtaacctcctccgccacattaaaCTG | 46L_47Q_49P_50I_52G_53F_54V_55C_67L_ | 3.756734884 |
| 16 | 2955 | TTCCAATGTGAGATCTGTGGGGCAAGCTTTactcagaaaggtaacctcctccgccacattaaaCTG | 46F_47Q_49E_50I_52G_53A_54S_55F_67L_ | 3.748420273 |
| 17 | 3117 | TTCCAATGTCCCGTATGTGGGGCAGTTTTTactcagaaaggtaacctcctccgccacattaaaCTG | 46F_47Q_49P_50V_52G_53A_54V_55F_67L_ | 3.745979959 |
| 18 | 2996 | TTCCAGTGTGAGGTCTGTGGGGCAACGTTTactcagaaaggtaacctcctccgccacattaaaCTG | 46F_47Q_49E_50V_52G_53A_54T_55F_67L_ | 3.744575269 |
| 19 | 2952 | TTCCAGTGTGAGATTTGTGGGGCACCATTTactcagaaaggtaacctcctccgccacattaaaCTG | 46F_47Q_49E_50I_52G_53A_54P_55F_67L_ | 3.722428107 |
| 20 | 2997 | TTCCAGTGTGAGGTATGTGGGGCAGTGTTTactcagaaaggtaacctcctccgccacattaaaCTG | 46F_47Q_49E_50V_52G_53A_54V_55F_67L_ | 3.710603856 |
| 21 | 3012 | TTCCAATGTAATATATGTGGGGCACCCTTTactcagaaaggtaacctcctccgccacattaaaCTG | 46F_47Q_49N_50I_52G_53A_54P_55F_67L_ | 3.708766019 |
| 22 | 2994 | TTCCAGTGTGAGGTGTGTGGGGCACGCTTTactcagaaaggtaacctcctccgccacattaaaCTG | 46F_47Q_49E_50V_52G_53A_54R_55F_67L_ | 3.706490633 |
| 23 | 2954 | TTCCAATGTGAGATCTGTGGGGCACGTTTTactcagaaaggtaacctcctccgccacattaaaCTG | 46F_47Q_49E_50I_52G_53A_54R_55F_67L_ | 3.704090899 |
| 24 | 2987 | TTCCAGTGTGAGGTCTGTGGGGCAATATTTactcagaaaggtaacctcctccgccacattaaaCTG | 46F_47Q_49E_50V_52G_53A_54I_55F_67L_ | 3.703445974 |
| 25 | 2983 | TTCCAATGTGAAGTCTGTGGGGCAGAGTTTactcagaaaggtaacctcctccgccacattaaaCTG | 46F_47Q_49E_50V_52G_53A_54E_55F_67L_ | 3.698174101 |
| 26 | 3065 | TTCCAGTGTCCCATTTGTGGGGCAGGTTTTactcagaaaggtaacctcctccgccacattaaaCTG | 46F_47Q_49P_50I_52G_53A_54G_55F_67L_ | 3.691376639 |
| 27 | 3100 | TTCCAGTGTCCAGTTTGTGGGGCAGCCTTTactcagaaaggtaacctcctccgccacattaaaCTG | 46F_47Q_49P_50V_52G_53A_54A_55F_67L_ | 3.688231654 |
| 28 | 2981 | TTCCAGTGTGAAGTGTGTGGGGCATGCTTTactcagaaaggtaacctcctccgccacattaaaCTG | 46F_47Q_49E_50V_52G_53A_54C_55F_67L_ | 3.686038028 |
| 29 | 2947 | TTCCAATGTGAGATCTGTGGGGCAATCTTTactcagaaaggtaacctcctccgccacattaaaCTG | 46F_47Q_49E_50I_52G_53A_54I_55F_67L_ | 3.675302068 |
| 30 | 3077 | TTCCAGTGTCCCATCTGTGGGGCAGTGTTTactcagaaaggtaacctcctccgccacattaaaCTG | 46F_47Q_49P_50I_52G_53A_54V_55F_67L_ | 3.671601149 |
| 31 | 6716 | CTGCAATGCCCCATCTGCGGGTTTACATGTactcagaaaggtaacctcctccgccacattaaaCTG | 46L_47Q_49P_50I_52G_53F_54T_55C_67L_ | 3.660541358 |
| 32 | 3072 | TTCCAATGTCCTATTTGTGGGGCACCATTTactcagaaaggtaacctcctccgccacattaaaCTG | 46F_47Q_49P_50I_52G_53A_54P_55F_67L_ | 3.650774907 |
| 33 | 3912 | TTCGTGTGTGAAATCTGTGGGGCACCCTTTactcagaaaggtaacctcctccgccacattaaaCTG | 46F_47V_49E_50I_52G_53A_54P_55F_67L_ | 3.650636194 |
| 34 | 2980 | TTCCAATGTGAAGTTTGTGGGGCAGCCTTTactcagaaaggtaacctcctccgccacattaaaCTG | 46F_47Q_49E_50V_52G_53A_54A_55F_67L_ | 3.649981999 |
| 35 | 7856 | CTGCAGTGCGAGATTTGTGGCTTCACATGCactcagaaaggtaacctcctccgccacattaaaCTG | 46L_47Q_49E_50I_52G_53F_54T_55C_67L_ | 3.649082629 |
| 36 | 3073 | TTCCAGTGTCCAATCTGTGGGGCACAGTTTactcagaaaggtaacctcctccgccacattaaaCTG | 46F_47Q_49P_50I_52G_53A_54Q_55F_67L_ | 3.648948145 |
| 37 | 2940 | TTCCAATGTGAAATCTGTGGGGCAGCATTTactcagaaaggtaacctcctccgccacattaaaCTG | 46F_47Q_49E_50I_52G_53A_54A_55F_67L_ | 3.643821427 |
| 38 | 3114 | TTCCAGTGTCCCGTATGTGGGGCAAGGTTTactcagaaaggtaacctcctccgccacattaaaCTG | 46F_47Q_49P_50V_52G_53A_54R_55F_67L_ | 3.633292745 |
| 39 | 6534 | CTCGAGTGTCCCATCTGCGGGTTTCGCTGCactcagaaaggtaacctcctccgccacattaaaCTG | 46L_47E_49P_50I_52G_53F_54R_55C_67L_ | 3.621447263 |
| 40 | 3232 | TTCAGGTGTGAGGTTTGTGGGGCACCGTTTactcagaaaggtaacctcctccgccacattaaaCTG | 46F_47R_49E_50V_52G_53A_54P_55F_67L_ | 3.617442303 |
| 41 | 3070 | TTCCAATGTCCAATCTGTGGGGCAATGTTTactcagaaaggtaacctcctccgccacattaaaCTG | 46F_47Q_49P_50I_52G_53A_54M_55F_67L_ | 3.615960468 |
| 42 | 6535 | CTAGAATGCCCCATCTGTGGTTTTTCGTGTactcagaaaggtaacctcctccgccacattaaaCTG | 46L_47E_49P_50I_52G_53F_54S_55C_67L_ | 3.615266726 |
| 43 | 3063 | TTCCAATGTCCAATTTGTGGGGCAGAGTTTactcagaaaggtaacctcctccgccacattaaaCTG | 46F_47Q_49P_50I_52G_53A_54E_55F_67L_ | 3.607281358 |
| 44 | 6701 | CTCCAATGCCCCATTTGTGGGTTCTGCTGTactcagaaaggtaacctcctccgccacattaaaCTG | 46L_47Q_49P_50I_52G_53F_54C_55C_67L_ | 3.60648233 |
| 45 | 3115 | TTCCAATGTCCTGTGTGTGGGGCATCATTTactcagaaaggtaacctcctccgccacattaaaCTG | 46F_47Q_49P_50V_52G_53A_54S_55F_67L_ | 3.601817006 |
| 46 | 2951 | TTCCAGTGTGAGATTTGTGGGGCAAACTTTactcagaaaggtaacctcctccgccacattaaaCTG | 46F_47Q_49E_50I_52G_53A_54N_55F_67L_ | 3.591308855 |
| 47 | 3113 | TTCCAGTGTCCCGTGTGTGGGGCACAGTTTactcagaaaggtaacctcctccgccacattaaaCTG | 46F_47Q_49P_50V_52G_53A_54Q_55F_67L_ | 3.589805764 |
| 48 | 6795 | CTGGTGTGCCCCATCTGCGGCTTCTCTTGTactcagaaaggtaacctcctccgccacattaaaCTG | 46L_47V_49P_50I_52G_53F_54S_55C_67L_ | 3.587385016 |
| 49 | 3110 | TTCCAGTGTCCAGTCTGTGGGGCAATGTTTactcagaaaggtaacctcctccgccacattaaaCTG | 46F_47Q_49P_50V_52G_53A_54M_55F_67L_ | 3.581623859 |
| 50 | 3071 | TTCCAGTGTCCTATTTGTGGGGCAAATTTTactcagaaaggtaacctcctccgccacattaaaCTG | 46F_47Q_49P_50I_52G_53A_54N_55F_67L_ | 3.579990305 |
| 51 | 7855 | TTACAGTGCGAAATATGCGGATTCAGCTGCactcagaaaggtaacctcctccgccacattaaaCTG | 46L_47Q_49E_50I_52G_53F_54S_55C_67L_ | 3.575648523 |
| 52 | 7854 | CTCCAGTGCGAGATCTGTGGTTTTAGATGTactcagaaaggtaacctcctccgccacattaaaCTG | 46L_47Q_49E_50I_52G_53F_54R_55C_67L_ | 3.575208659 |
| 53 | 3192 | TTCCGCTGTGAAATCTGTGGGGCACCCTTTactcagaaaggtaacctcctccgccacattaaaCTG | 46F_47R_49E_50I_52G_53A_54P_55F_67L_ | 3.575102785 |
| 54 | 2950 | TTCCAGTGTGAGATCTGTGGGGCAATGTTTactcagaaaggtaacctcctccgccacattaaaCTG | 46F_47Q_49E_50I_52G_53A_54M_55F_67L_ | 3.574297826 |
| 55 | 2991 | TTCCAGTGTGAAGTTTGTGGGGCAAATTTTactcagaaaggtaacctcctccgccacattaaaCTG | 46F_47Q_49E_50V_52G_53A_54N_55F_67L_ | 3.561330152 |
| 56 | 3076 | TTCCAGTGTCCAATATGTGGGGCAACCTTTactcagaaaggtaacctcctccgccacattaaaCTG | 46F_47Q_49P_50I_52G_53A_54T_55F_67L_ | 3.559693741 |
| 57 | 6780 | CTCGTTTGCCCAATTTGTGGGTTTGCTTGTactcagaaaggtaacctcctccgccacattaaaCTG | 46L_47V_49P_50I_52G_53F_54A_55C_67L_ | 3.558077994 |
| 58 | 2953 | TTCCAGTGTGAGATTTGTGGGGCACAGTTTactcagaaaggtaacctcctccgccacattaaaCTG | 46F_47Q_49E_50I_52G_53A_54Q_55F_67L_ | 3.557223316 |
| 59 | 6710 | CTGCAATGTCCAATCTGTGGCTTTATGTGCactcagaaaggtaacctcctcogccacattaaaCTG | 46L_47Q_49P_50I_52G_53F_54M_55C_67L_ | 3.553152041 |
| 60 | 6700 | CTCCAGTGCCCGATTTGTGGGTTTGCTTGTactcagaaaggtaacctcctccgccacattaaaCTG | 46L_47Q_49P_50I_52G_53F_54A_55C_67L_ | 3.551099882 |
| 61 | 794 | TTCGAGTGTGAAATCTGTGGGGCACGCTTTactcagaaaggtaacctcctccgccacattaaaCTG | 46F_47E_49E_50I_52G_53A_54R_55F_67L_ | 3.549299695 |
| 62 | 3074 | TTCCAATGTCCAATTTGTGGGGCAAGGTTTactcagaaaggtaacctcctccgccacattaaaCTG | 46F_47Q_49P_50I_52G_53A_54R_55F_67L_ | 3.548975408 |
| 63 | 3955 | TTCGTATGTGAGGTTTGTGGGGCATCCTTTactcagaaaggtaacctcctccgccacattaaaCTG | 46F_47V_49E_50V_52G_53A_54S_55F_67L_ | 3.539523124 |
| 64 | 2992 | TTCCAATGTGAGGTCTGTGGGGCACCATTTactcagaaaggtaacctcctccgccacattaaaCTG | 46F_47Q_49E_50V_52G_53A_54P_55F_67L_ | 3.534672317 |
| 65 | 2949 | TTCCAGTGTGAAATATGTGGGGCACTTTTTactcagaaaggtaacctcctccgccacattaaaCTG | 46F_47Q_49E_50I_52G_53A_54L_55F_67L_ | 3.534211388 |
| 66 | 6715 | TTACAGTGTCCAATCTGCGGATTTAGCTGTactcagaaaggtaacctcctccgccacattaaaCTG | 46L_47Q_49P_50I_52G_53F_54S_55C_67L_ | 3.532460778 |
| 67 | 2995 | TTCCAGTGTGAGGTCTGTGGGGCATCCTTTactcagaaaggtaacctcctccgccacattaaaCTG | 46F_47Q_49E_50V_52G_53A_54S_55F_67L_ | 3.531018064 |
| 68 | 3060 | TTCCAATGTCCTATATGTGGGGCAGCTTTTactcagaaaggtaacctcctccgccacattaaaCTG | 46F_47Q_49P_50I_52G_53A_54A_55F_67L_ | 3.528773155 |
| 69 | 3312 | TTCCGCTGTCCCATCTGTGGGGCACCATTTactcagaaaggtaacctcctccgccacattaaaCTG | 46F_47R_49P_50I_52G_53A_54P_55F_67L_ | 3.528054369 |
| 70 | 3107 | TTCCAGTGTCCAGTGTGTGGGGCAATTTTTactcagaaaggtaacctcctccgccacattaaaCTG | 46F_47Q_49P_50V_52G_53A_54I_55F_67L_ | 3.527490423 |
| 71 | 2943 | TTCCAATGTGAGATCTGTGGGGCAGAATTTactcagaaaggtaacctcctccgccacattaaaCTG | 46F_47Q_49E_50I_52G_53A_54E_55F_67L_ | 3.525182531 |
| 72 | 2990 | TTCCAGTGTGAGGTGTGTGGGGCAATGTTTactcagaaaggtaacctcctccgccacattaaaCTG | 46F_47Q_49E_50V_52G_53A_54M_55F_67L_ | 3.522008423 |
| 73 | 3132 | TTCAGGTGTGATATCTGTGGGGCACCCTTTactcagaaaggtaacctcctccgccacattaaaCTG | 46F_47R_49D_50I_52G_53A_54P_55F_67L_ | 3.521460644 |
| 74 | 3352 | TTCCGCTGTCCCGTTTGTGGGGCACCATTTactcagaaaggtaacctcctccgccacattaaaCTG | 46F_47R_49P_50V_52G_53A_54P_55F_67L_ | 3.513180079 |
| 75 | 3109 | TTCCAATGTCCTGTCTGTGGGGCACTGTTTactcagaaaggtaacctcctccgccacattaaaCTG | 46F_47Q_49P_50V_52G_53A_54L_55F_67L_ | 3.497342287 |
| 76 | 6775 | CTCACCTGCCCAATCTGCGGGTTCAGCTGTactcagaaaggtaacctcctccgccacattaaaCTG | 46L_47T_49P_50I_52G_53F_54S_55C_67L_ | 3.494800751 |
| 77 | 4032 | TTCGTGTGTCCTATCTGTGGGGCACCTTTTactcagaaaggtaacctcctccgccacattaaaCTG | 46F_47V_49P_50I_52G_53A_54P_55F_67L_ | 3.492650231 |
| 78 | 3111 | TTCCAATGTCCGGTTTGTGGGGCAAACTTTactcagaaaggtaacctcctccgccacattaaaCTG | 46F_47Q_49P_50V_52G_53A_54N_55F_67L_ | 3.483776158 |
| 79 | 2941 | TTCCAATGTGAAATCTGTGGGGCATGTTTTactcagaaaggtaacctcctccgccacattaaaCTG | 46F_47Q_49E_50I_52G_53A_54C_55F_67L_ | 3.477323463 |
| 80 | 3017 | TTCCAATGTAACATATGTGGGGCAGTGTTTactcagaaaggtaacctcctccgccacattaaaCTG | 46F_47Q_49N_50I_52G_53A_54V_55F_67L_ | 3.473136781 |
| 81 | 6719 | CTGCAGTGCCCGATCTGCGGGTTCTACTGCactcagaaaggtaacctcctccgccacattaaaCTG | 46L_47Q_49P_50I_52G_53F_54Y_55C_67L_ | 3.46909174 |
| 82 | 2887 | TTCCAGTGTGACATCTGTGGGGCAATCTTTactcagaaaggtaacctcctccgccacattaaaCTG | 46F_47Q_49D_50I_52G_53A_54I_55F_67L_ | 3.467254877 |
| 83 | 3101 | TTCCAATGTCCTGTCTGTGGGGCATGCTTTactcagaaaggtaacctcctccgccacattaaaCTG | 46F_47Q_49P_50V_52G_53A_54C_55F_67L_ | 3.464943806 |
| 84 | 2948 | TTCCAGTGTGAAATTTGTGGGGCAAAGTTTactcagaaaggtaacctcctccgccacattaaaCTG | 46F_47Q_49E_50I_52G_53A_54K_55F_67L_ | 3.461174457 |
| 85 | 6707 | TTGCAGTGCCCAATTTGCGGTTTCATTTGTactcagaaaggtaacctcctccgccacattaaaCTG | 46L_47Q_49P_50I_52G_53F_54I_55C_67L_ | 3.453318605 |
| 86 | 6713 | CTACAGTGCCCCATTTGCGGTTTCCAGTGCactcagaaaggtaacctcctccgccacattaaaCTG | 46L_47Q_49P_50I_52G_53F_54Q_55C_67L_ | 3.448638586 |
| 87 | 6776 | CTGACGTGTCCCATTTGTGGGTTTACGTGTactcagaaaggtaacctcctccgccacattaaaCTG | 46L_47T_49P_50I_52G_53F_54T_55C_67L_ | 3.444282477 |
| 88 | 6703 | CTGCAGTGCCCCATTTGCGGGTTCGAGTGTactcagaaaggtaacctcctccgccacattaaaCTG | 46L_47Q_49P_50I_52G_53F_54E_55C_67L_ | 3.442282622 |
| 89 | 6732 | CTGAGATGTCCTATTTGTGGCTTTCCCTGCactcagaaaggtaacctcctccgccacattaaaCTG | 46L_47R_49P_50I_52G_53F_54P_55C_67L_ | 3.438295777 |
| 90 | 3068 | TTCCAGTGTCCCATCTGTGGGGCAAAATTTactcagaaaggtaacctcctccgccacattaaaCTG | 46F_47Q_49P_50I_52G_53A_54K_55F_67L_ | 3.435535315 |
| 91 | 1752 | TTCATCTGTGAGATTTGTGGGGCACCTTTTactcagaaaggtaacctcctccgccacattaaaCTG | 46F_47I_49E_50I_52G_53A_54P_55F_67L_ | 3.432727236 |
| 92 | 3097 | TTCCAGTGTCCGCAGTGTGGGGCAGTTTTTactcagaaaggtaacctcctccgccacattaaaCTG | 46F_47Q_49P_50Q_52G_53A_54V_55F_67L_ | 3.423210654 |
| 93 | 2392 | TTCCTGTGTCCCGTCTGTGGGGCACCTTTTactcagaaaggtaacctcctccgccacattaaaCTG | 46F_47L_49P_50V_52G_53A_54P_55F_67L_ | 3.4141742 |
| 94 | 3057 | TTCCAGTGTAACGTTTGTGGGGCAGTATTTactcagaaaggtaacctcctccgccacattaaaCTG | 46F_47Q_49N_50V_52G_53A_54V_55F_67L_ | 3.408663984 |
| 95 | 7840 | CTCCAGTGTGAGATATGTGGGTTTGCATGCactcagaaaggtaacctcctccgccacattaaaCTG | 46L_47Q_49E_50I_52G_53F_54A_55C_67L_ | 3.402238927 |
| 96 | 6708 | CTGCAGTGTCCGATCTGTGGTTTCAAGTGTactcagaaaggtaacctcctccgccacattaaaCTG | 46L_47Q_49P_50I_52G_53F_54K_55C_67L_ | 3.401245222 |
| 97 | 2988 | TTCCAATGTGAGGTGTGTGGGGCAAAGTTTactcagaaaggtaacctcctccgccacattaaaCTG | 46F_47Q_49E_50V_52G_53A_54K_55F_67L_ | 3.401068126 |
| 98 | 3102 | TTCCAGTGTCCAGTCTGTGGGGCAGACTTTactcagaaaggtaacctcctccgccacattaaaCTG | 46F_47Q_49P_50V_52G_53A_54D_55F_67L_ | 3.400114462 |
| 99 | 7857 | CTGCAGTGCGAAATCTGCGGCTTTGTCTGCactcagaaaggtaacctcctccgccacattaaaCTG | 46L_47Q_49E_50I_52G_53F_54V_55C_67L_ | 3.39741922 |
| 100 | 3000 | TTCCAGTGTAATATTTGTGGGGCAGCATTTactcagaaaggtaacctcctccgccacattaaaCTG | 46F_47Q_49N_50I_52G_53A_54A_55F_67L_ | 3.386293654 |
| 101 | 6536 | CTGGAATGTCCGATTTGTGGCTTTACCTGCactcagaaaggtaacctcctccgccacattaaaCTG | 46L_47E_49P_50I_52G_53F_54T_55C_67L_ | 3.382376875 |
| 102 | 6496 | TTGTGCTGTCCGATATGTGGATTCACCTGTactcagaaaggtaacctcctccgccacattaaaCTG | 46L_47C_49P_50I_52G_53F_54T_55C_67L_ | 3.382276384 |
| 103 | 3105 | TTCCAGTGTCCCGTGTGTGGGGCAGGCTTTactcagaaaggtaacctcctccgccacattaaaCTG | 46F_47Q_49P_50V_52G_53A_54G_55F_67L_ | 3.381169998 |
| 104 | 3252 | TTCAGGTGTAACATTTGTGGGGCACCGTTTactcagaaaggtaacctcctccgccacattaaaCTG | 46F_47R_49N_50I_52G_53A_54P_55F_67L_ | 3.379977756 |
| 105 | 3001 | TTCCAATGTAACATATGTGGGGCATGTTTTactcagaaaggtaacctcctccgccacattaaaCTG | 46F_47Q_49N_50I_52G_53A_54C_55F_67L_ | 3.374981255 |
| 106 | 3047 | TTCCAATGTAACGTGTGTGGGGCAATTTTTactcagaaaggtaacctcctccgccacattaaaCTG | 46F_47Q_49N_50V_52G_53A_54I_55F_67L_ | 3.374297713 |
| 107 | 3069 | TTCCAATGTCCAATTTGTGGGGCACTCTTTactcagaaaggtaacctcctccgccacattaaaCTG | 46F_47Q_49P_50I_52G_53A_54L_55F_67L_ | 3.372744842 |
| 108 | 914 | TTCGAATGTCCTATTTGTGGGGCACGCTTTactcagaaaggtaacctcctccgccacattaaaCTG | 46F_47E_49P_50I_52G_53A_54R_55F_67L_ | 3.366876166 |
| 109 | 3079 | TTCCAGTGTCCAATCTGTGGGGCATATTTTactcagaaaggtaacctcctccgccacattaaaCTG | 46F_47Q_49P_50I_52G_53A_54Y_55F_67L_ | 3.361334344 |
| 110 | 3940 | TTCGTCTGTGAAGTGTGTGGGGCAGCATTTactcagaaaggtaacctcctccgccacattaaaCTG | 46F_47V_49E_50V_52G_53A_54A_55F_67L_ | 3.355307226 |
| 111 | 6714 | CTGCAGTGTCCAATCTGTGGATTCAGGTGCactcagaaaggtaacctcctccgccacattaaaCTG | 46L_47Q_49P_50I_52G_53F_54R_55C_67L_ | 3.346255154 |
| 112 | 2892 | TTCCAGTGTGATATTTGTGGGGCACCCTTTactcagaaaggtaacctcctccgccacattaaaCTG | 46F_47Q_49D_50I_52G_53A_54P_55F_67L_ | 3.345934903 |
| 113 | 6539 | CTGGAGTGCCCAATCTGTGGATTCTACTGCactcagaaaggtaacctcctccgccacattaaaCTG | 46L_47E_49P_50I_52G_53F_54Y_55C_67L_ | 3.343125374 |
| 114 | 3015 | TTCCAGTGTAATATTTGTGGGGCATCATTTactcagaaaggtaacctcctccgccacattaaaCTG | 46F_47Q_49N_50I_52G_53A_54S_55F_67L_ | 3.343063271 |
| 115 | 3972 | TTCGTCTGTAACATTTGTGGGGCACCCTTTactcagaaaggtaacctcctccgccacattaaaCTG | 46F_47V_49N_50I_52G_53A_54P_55F_67L_ | 3.331900195 |
| 116 | 917 | TTCGAGTGTCCAATCTGTGGGGCAGTCTTTactcagaaaggtaacctcctccgccacattaaaCTG | 46F_47E_49P_50I_52G_53A_54V_55F_67L_ | 3.330969334 |
| 117 | 3821 | TTCACTTGTCCAGTGTGTGGGGCATGTTTTactcagaaaggtaacctcctccgccacattaaaCTG | 46F_47T_49P_50V_52G_53A_54C_55F_67L_ | 3.329685775 |
| 118 | 7935 | TTAGTGTGCGAAATCTGCGGCTTCTCCTGCactcagaaaggtaacctcctccgccacattaaaCTG | 46L_47V_49E_50I_52G_53F_54S_55C_67L_ | 3.326030061 |
| 119 | 915 | TTCGAGTGTCCAATCTGTGGGGCATCTTTTactcagaaaggtaacctcctccgccacattaaaCTG | 46F_47E_49P_50I_52G_53A_54S_55F_67L_ | 3.324744415 |
| 120 | 3066 | TTCCAGTGTCCTATTTGTGGGGCACATTTTactcagaaaggtaacctcctccgccacattaaaCTG | 46F_47Q_49P_50I_52G_53A_54H_55F_67L_ | 3.317967633 |
| 121 | 3799 | TTCACTTGTCCAATCTGTGGGGCATATTTTactcagaaaggtaacctcctccgccacattaaaCTG | 46F_47T_49P_50I_52G_53A_54Y_55F_67L_ | 3.30986861 |
| 122 | 6706 | CTCCAGTGCCCGATCTGCGGATTTCACTGCactcagaaaggtaacctcctccgccacattaaaCTG | 46L_47Q_49P_50I_52G_53F_54H_55C_67L_ | 3.304828979 |
| 123 | 2881 | TTCCAATGTGATATATGTGGGGCATGTTTTactcagaaaggtaacctcctccgccacattaaaCTG | 46F_47Q_49D_50I_52G_53A_54C_55F_67L_ | 3.298730533 |
| 124 | 7853 | CTGCAGTGTGAGATTTGCGGTTTCCAATGTactcagaaaggtaacctcctccgccacattaaaCTG | 46L_47Q_49E_50I_52G_53F_54Q_55C_67L_ | 3.29751284 |
| 125 | 834 | TTCGAGTGTGAAGTTTGTGGGGCAAGGTTTactcagaaaggtaacctcctccgccacattaaaCTG | 46F_47E_49E_50V_52G_53A_54R_55F_67L_ | 3.29184016 |
| 126 | 2888 | TTCCAGTGTGATATCTGTGGGGCAAAGTTTactcagaaaggtaacctcctccgccacattaaaCTG | 46F_47Q_49D_50I_52G_53A_54K_55F_67L_ | 3.282141906 |
| 127 | 3013 | TTCCAGTGTAATATTTGTGGGGCACAATTTactcagaaaggtaacctcctccgccacattaaaCTG | 46F_47Q_49N_50I_52G_53A_54Q_55F_67L_ | 3.279013332 |
| 128 | 7934 | CTCGTCTGCGAGATTTGTGGCTTCAGATGCactcagaaaggtaacctcctccgccacattaaaCTG | 46L_47V_49E_50I_52G_53F_54R_55C_67L_ | 3.277456934 |
| 129 | 3080 | TTCCAATGTCCCCAGTGTGGGGCAGCATTTactcagaaaggtaacctcctccgccacattaaaCTG | 46F_47Q_49P_50Q_52G_53A_54A_55F_67L_ | 3.273372389 |
| 130 | 911 | TTCGAATGTCCCATATGTGGGGCAAATTTTactcagaaaggtaacctcctccgccacattaaaCTG | 46F_47E_49P_50I_52G_53A_54N_55F_67L_ | 3.26878841 |
| 131 | 7848 | CTGCAATGCGAAATTTGTGGATTCAAGTGCactcagaaaggtaacctcctccgccacattaaaCTG | 46L_47Q_49E_50I_52G_53F_54K_55C_67L_ | 3.266800915 |
| 132 | 3106 | TTCCAGTGTCCCGTGTGTGGGGCACACTTTactcagaaaggtaacctcctccgccacattaaaCTG | 46F_47Q_49P_50V_52G_53A_54H_55F_67L_ | 3.265847613 |
| 133 | 3014 | TTCCAGTGTAATATCTGTGGGGCACGCTTTactcagaaaggtaacctcctccgccacattaaaCTG | 46F_47Q_49N_50I_52G_53A_54R_55F_67L_ | 3.261630997 |
| 134 | 3008 | TTCCAGTGTAATATTTGTGGGGCAAAGTTTactcagaaaggtaacctcctccgccacattaaaCTG | 46F_47Q_49N_50I_52G_53A_54K_55F_67L_ | 3.259052831 |
| 135 | 6528 | TTAGAATGCCCTATCTGTGGCTTTAAATGCactcagaaaggtaacctcctccgccacattaaaCTG | 46L_47E_49P_50I_52G_53F_54K_55C_67L_ | 3.258451021 |
| 136 | 3714 | TTCACATGTGAGGTGTGTGGGGCAAGATTTactcagaaaggtaacctcctccgccacattaaaCTG | 46F_47T_49E_50V_52G_53A_54R_55F_67L_ | 3.25580671 |
| 137 | 2956 | TTCCAATGTGAGATTTGTGGGGCAACATTTactcagaaaggtaacctcctccgccacattaaaCTG | 46F_47Q_49E_50I_52G_53A_54T_55F_67L_ | 3.255800612 |
| 138 | 3081 | TTCCAGTGTCCTCAGTGTGGGGCATGTTTTactcagaaaggtaacctcctccgccacattaaaCTG | 46F_47Q_49P_50Q_52G_53A_54C_55F_67L_ | 3.255190746 |
| 139 | 3954 | TTCGTATGTGAGGTCTGTGGGGCACGATTTactcagaaaggtaacctcctccgccacattaaaCTG | 46F_47V_49E_50V_52G_53A_54R_55F_67L_ | 3.25402995 |
| 140 | 3792 | TTCACATGTCCCATCTGTGGGGCACCGTTTactcagaaaggtaacctcctccgccacattaaaCTG | 46F_47T_49P_50I_52G_53A_54P_55F_67L_ | 3.253492125 |
| 141 | 2896 | TTCCAGTGTGATATCTGTGGGGCAACGTTTactcagaaaggtaacctcctccgccacattaaaCTG | 46F_47Q_49D_50I_52G_53A_54T_55F_67L_ | 3.248610108 |
| 142 | 3003 | TTCCAGTGTAACATCTGTGGGGCAGAATTTactcagaaaggtaacctcctccgccacattaaaCTG | 46F_47Q_49N_50I_52G_53A_54E_55F_67L_ | 3.243118819 |
| 143 | 7755 | CTTATCTGTGAGATATGCGGATTTTCCTGCactcagaaaggtaacctcctccgccacattaaaCTG | 46L_47I_49E_50I_52G_53F_54S_55C_67L_ | 3.242603579 |
| 144 | 6499 | CTATGCTGTCCTATTTGTGGTTTCTACTGTactcagaaaggtaacctcctccgccacattaaaCTG | 46L_47C_49P_50I_52G_53F_54Y_55C_67L_ | 3.240335146 |
| 145 | 6779 | CTAACCTGTCCCATATGTGGTTTTTATTGCactcagaaaggtaacctcctccgccacattaaaCTG | 46L_47T_49P_50I_52G_53F_54Y_55C_67L_ | 3.236030294 |
| 146 | 2959 | TTCCAGTGTGAGATTTGTGGGGCATATTTTactcagaaaggtaacctcctccgccacattaaaCTG | 46F_47Q_49E_50I_52G_53A_54Y_55F_67L_ | 3.234022813 |
| 147 | 3674 | TTCACCTGTGAAATTTGTGGGGCAAGGTTTactcagaaaggtaacctcctccgccacattaaaCTG | 46F_47T_49E_50I_52G_53A_54R_55F_67L_ | 3.232869148 |
| 148 | 6705 | CTGCAGTGTCCTATCTGCGGTTTCGGCTGTactcagaaaggtaacctcctccgccacattaaaCTG | 46L_47Q_49P_50I_52G_53F_54G_55C_67L_ | 3.230130714 |
| 149 | 3795 | TTCACTTGTCCAATCTGTGGGGCAAGCTTTactcagaaaggtaacctcctccgccacattaaaCTG | 46F_47T_49P_50I_52G_53A_54S_55F_67L_ | 3.228402875 |
| 150 | 6709 | CTCCAGTGTCCCATTTGCGGATTCCTGTGTactcagaaaggtaacctcctccgccacattaaaCTG | 46L_47Q_49P_50I_52G_53F_54L_55C_67L_ | 3.22808616 |
| 151 | 3905 | TTCGTGTGTGAAATTTGTGGGGCAGGTTTTactcagaaaggtaacctcctccgccacattaaaCTG | 46F_47V_49E_50I_52G_53A_54G_55F_67L_ | 3.226910676 |
| 152 | 3913 | TTCGTGTGTGAGATCTGTGGGGCACAGTTTactcagaaaggtaacctcctccgccacattaaaCTG | 46F_47V_49E_50I_52G_53A_54Q_55F_67L_ | 3.223715656 |
| 153 | 2957 | TTCCAGTGTGAAATTTGTGGGGCAGTTTTTactcagaaaggtaacctcctccgccacattaaaCTG | 46F_47Q_49E_50I_52G_53A_54V_55F_67L_ | 3.221983198 |
| 154 | 6796 | TTAGTCTGCCCTATCTGCGGTTTTACCTGCactcagaaaggtaacctcctccgccacattaaaCTG | 46L_47V_49P_50I_52G_53F_54T_55C_67L_ | 3.214758862 |
| 155 | 7847 | CTCCAGTGTGAGATCTGTGGTTTCATATGCactcagaaaggtaacctcctccgccacattaaaCTG | 46L_47Q_49E_50I_52G_53F_54I_55C_67L_ | 3.214079391 |
| 156 | 2985 | TTCCAGTGTGAGGTTTGTGGGGCAGGTTTTactcagaaaggtaacctcctccgccacattaaaCTG | 46F_47Q_49E_50V_52G_53A_54G_55F_67L_ | 3.21360118 |
| 157 | 3041 | TTCCAATGTAACGTGTGTGGGGCATGTTTTactcagaaaggtaacctcctccgccacattaaaCTG | 46F_47Q_49N_50V_52G_53A_54C_55F_67L_ | 3.208398501 |
| 158 | 2989 | TTCCAGTGTGAGGTCTGTGGGGCACTGTTTactcagaaaggtaacctcctccgccacattaaaCTG | 46F_47Q_49E_50V_52G_53A_54L_55F_67L_ | 3.208344191 |
| 159 | 7936 | CTGGTGTGTGAAATCTGCGGGTTCACATGTactcagaaaggtaacctcctccgccacattaaaCTG | 46L_47V_49E_50I_52G_53F_54T_55C_67L_ | 3.204960208 |
| 160 | 6794 | TTGGTCTGTCCAATTTGCGGGTTCCGATGTactcagaaaggtaacctcctccgccacattaaaCTG | 46L_47V_49P_50I_52G_53F_54R_55C_67L_ | 3.202416834 |
| 161 | 7915 | TTGACTTGCGAGATATGTGGATTCTCCTGCactcagaaaggtaacctcctccgccacattaaaCTG | 46L_47T_49E_50I_52G_53F_54S_55C_67L_ | 3.195870557 |
| 162 | 4072 | TTCGTTTGTCCGGTCTGTGGGGCACCATTTactcagaaaggtaacctcctccgccacattaaaCTG | 46F_47V_49P_50V_52G_53A_54P_55F_67L_ | 3.194957951 |
| 163 | 6615 | TTGATCTGCCCAATTTGCGGCTTCTCCTGCactcagaaaggtaacctcctccgccacattaaaCTG | 46L_47I_49P_50I_52G_53F_54S_55C_67L_ | 3.184850812 |
| 164 | 2352 | TTCTTATGTCCCATCTGTGGGGCACCCTTTactcagaaaggtaacctcctccgccacattaaaCTG | 46F_47L_49P_50I_52G_53A_54P_55F_67L_ | 3.184050761 |
| 165 | 6785 | TTGGTGTGCCCCATATGTGGGTTCGGGTGTactcagaaaggtaacctcctccgccacattaaaCTG | 46L_47V_49P_50I_52G_53F_54G_55C_67L_ | 3.182664373 |
| 166 | 6327 | TTCCAGTGCCCAATCTGCGGCCGAATCTTTactcagaaaggtaacctcctccgccacattaaaCTG | 46F_47Q_49P_50I_52G_53R_54I_55F_67L_ | 3.177105385 |
| 167 | 3096 | TTCCAGTGTCCTCAGTGTGGGGCAACATTTactcagaaaggtaacctcctccgccacattaaaCTG | 46F_47Q_49P_50Q_52G_53A_54T_55F_67L_ | 3.174353778 |
| 168 | 2946 | TTCCAGTGTGAAATCTGTGGGGCACATTTTactcagaaaggtaacctcctccgccacattaaaCTG | 46F_47Q_49E_50I_52G_53A_54H_55F_67L_ | 3.171641367 |
| 169 | 7850 | CTTCAGTGCGAGATATGCGGATTTATGTGCactcagaaaggtaacctcctccgccacattaaaCTG | 46L_47Q_49E_50I_52G_53F_54M_55C_67L_ | 3.171233827 |
| 170 | 3672 | TTCACGTGTGAGATCTGTGGGGCACCCTTTactcagaaaggtaacctcctccgccacattaaaCTG | 46F_47T_49E_50I_52G_53A_54P_55F_67L_ | 3.170412699 |
| 171 | 6520 | CTGGAGTGTCCCATCTGCGGGTTCGCTTGCactcagaaaggtaacctcctccgccacattaaaCTG | 46L_47E_49P_50I_52G_53F_54A_55C_67L_ | 3.169772208 |
| 172 | 2897 | TTCCAGTGTGATATTTGTGGGGCAGTATTTactcagaaaggtaacctcctccgccacattaaaCTG | 46F_47Q_49D_50I_52G_53A_54V_55F_67L_ | 3.169064342 |
| 173 | 1792 | TTCATCTGTGAGGTGTGTGGGGCACCATTTactcagaaaggtaacctcctccgccacattaaaCTG | 46F_47I_49E_50V_52G_53A_54P_55F_67L_ | 3.168087388 |
| 174 | 2232 | TTCCTGTGTGAAATCTGTGGGGCACCATTTactcagaaaggtaacctcctccgccacattaaaCTG | 46F_47L_49E_50I_52G_53A_54P_55F_67L_ | 3.167959837 |
| 175 | 951 | TTCGAATGTCCGGTTTGTGGGGCAAATTTTactcagaaaggtaacctcctccgccacattaaaCTG | 46F_47E_49P_50V_52G_53A_54N_55F_67L_ | 3.164232705 |
| 176 | 1753 | TTCATTTGTGAAATTTGTGGGGCACAATTTactcagaaaggtaacctcctccgccacattaaaCTG | 46F_47I_49E_50I_52G_53A_54Q_55F_67L_ | 3.163895203 |
| 177 | 1872 | TTCATCTGTCCTATTTGTGGGGCACCTTTTactcagaaaggtaacctcctccgccacattaaaCTG | 46F_47I_49P_50I_52G_53A_54P_55F_67L_ | 3.163373848 |
| 178 | 6533 | TTAGAATGCCCTATCTGCGGTTTCCAATGCactcagaaaggtaacctcctccgccacattaaaCTG | 46L_47E_49P_50I_52G_53F_54Q_55C_67L_ | 3.15685874 |
| 179 | 2936 | TTCCAGTGTGATGTTTGTGGGGCAACTTTTactcagaaaggtaacctcctccgccacattaaaCTG | 46F_47Q_49D_50V_52G_53A_54T_55F_67L_ | 3.155787354 |
| 180 | 3900 | TTCGTGTGTGAAATCTGTGGGGCAGCATTTactcagaaaggtaacctcctccgccacattaaaCTG | 46F_47V_49E_50I_52G_53A_54A_55F_67L_ | 3.154626909 |
| 181 | 7674 | CTCGAGTGCGAAATTTGTGGATTCCGGTGCactcagaaaggtaacctcctccgccacattaaaCTG | 46L_47E_49E_50I_52G_53F_54R_55C_67L_ | 3.151982382 |
| 182 | 7916 | CTGACCTGCGAAATCTGCGGATTTACTTGCactcagaaaggtaacctcctccgccacattaaaCTG | 46L_47T_49E_50I_52G_53F_54T_55C_67L_ | 3.136183156 |
| 183 | 6704 | TTACAATGTCCTATCTGCGGATTTTTTTGTactcagaaaggtaacctcctccgccacattaaaCTG | 46L_47Q_49P_50I_52G_53F_54F_55C_67L_ | 3.135172653 |
| 184 | 7756 | CTCATCTGCGAGATCTGTGGATTCACCTGTactcagaaaggtaacctcctccgccacattaaaCTG | 46L_47I_49E_50I_52G_53F_54T_55C_67L_ | 3.132402399 |
| 185 | 7933 | CTGGTGTGTGAAATATGCGGCTTCCAATGTactcagaaaggtaacctcctccgccacattaaaCTG | 46L_47V_49E_50I_52G_53F_54Q_55C_67L_ | 3.131724152 |
| 186 | 7841 | CTGCAGTGCGAAATTTGTGGCTTCTGCTGTactcagaaaggtaacctcctccgccacattaaaCTG | 46L_47Q_49E_50I_52G_53F_54C_55C_67L_ | 3.126672373 |
| 187 | 3715 | TTCACATGTGAAGTGTGTGGGGCATCGTTTactcagaaaggtaacctcctccgccacattaaaCTG | 46F_47T_49E_50V_52G_53A_54S_55F_67L_ | 3.12070437 |
| 188 | 6712 | CTGCAATGCCCTATTTGTGGCTTTCCGTGCactcagaaaggtaacctcctccgccacattaaaCTG | 46L_47Q_49P_50I_52G_53F_54P_55C_67L_ | 3.118857948 |
| 189 | 6495 | CTCTGCTGCCCCATCTGTGGTTTCAGTTGCactcagaaaggtaacctcctccgccacattaaaCTG | 46L_47C_49P_50I_52G_53F_54S_55C_67L_ | 3.114888322 |
| 190 | 900 | TTCGAGTGTCCAATCTGTGGGGCAGCGTTTactcagaaaggtaacctcctccgccacattaaaCTG | 46F_47E_49P_50I_52G_53A_54A_55F_67L_ | 3.110759616 |
| 191 | 2976 | TTCCAGTGTGAGCAGTGTGGGGCAACCTTTactcagaaaggtaacctcctccgccacattaaaCTG | 46F_47Q_49E_50Q_52G_53A_54T_55F_67L_ | 3.097942255 |
| 192 | 3040 | TTCCAGTGTAACGTGTGTGGGGCAGCATTTactcagaaaggtaacctcctccgccacattaaaCTG | 46F_47Q_49N_50V_52G_53A_54A_55F_67L_ | 3.094419002 |
| 193 | 2982 | TTCCAATGTGAAGTGTGTGGGGCAGACTTTactcagaaaggtaacctcctccgccacattaaaCTG | 46F_47Q_49E_50V_52G_53A_54D_55F_67L_ | 3.091190687 |
| 194 | 7920 | TTAGTGTGCGAGATTTGTGGGTTCGCCTGCactcagaaaggtaacctcctccgccacattaaaCTG | 46L_47V_49E_50I_52G_53F_54A_55C_67L_ | 3.091038172 |
| 195 | 6793 | CTGGTCTGCCCTATCTGCGGGTTTCAGTGCactcagaaaggtaacctcctccgccacattaaaCTG | 46L_47V_49P_50I_52G_53F_54Q_55C_67L_ | 3.088925734 |
| 196 | 2945 | TTCCAGTGTGAAATCTGTGGGGCAGGCTTTactcagaaaggtaacctcctccgccacattaaaCTG | 46F_47Q_49E_50I_52G_53A_54G_55F_67L_ | 3.087597464 |
| 197 | 1740 | TTCATTTGTGAAATCTGTGGGGCAGCATTTactcagaaaggtaacctcctccgccacattaaaCTG | 46F_47I_49E_50I_52G_53A_54A_55F_67L_ | 3.085929703 |
| 198 | 3019 | TTCCAATGTAACATTTGTGGGGCATATTTTactcagaaaggtaacctcctccgccacattaaaCTG | 46F_47Q_49N_50I_52G_53A_54Y_55F_67L_ | 3.084849782 |
| 199 | 3011 | TTCCAGTGTAACATATGTGGGGCAAATTTTactcagaaaggtaacctcctccgccacattaaaCTG | 46F_47Q_49N_50I_52G_53A_54N_55F_67L_ | 3.084268775 |
| 200 | 4035 | TTCGTCTGTCCTATCTGTGGGGCAAGTTTTactcagaaaggtaacctcctccgccacattaaaCTG | 46F_47V_49P_50I_52G_53A_54S_55F_67L_ | 3.081814695 |
| 201 | 1912 | TTCATCTGTCCAGTCTGTGGGGCACCCTTTactcagaaaggtaacctcctccgccacattaaaCTG | 46F_47I_49P_50V_52G_53A_54P_55F_67L_ | 3.078938702 |
| 202 | 314 | TTCTGCTGTGAAATATGTGGGGCACGTTTTactcagaaaggtaacctcctccgccacattaaaCTG | 46F_47C_49E_50I_52G_53A_54R_55F_67L_ | 3.078924086 |
| 203 | 4060 | TTCGTGTGTCCTGTCTGTGGGGCAGCGTTTactcagaaaggtaacctcctccgccacattaaaCTG | 46F_47V_49P_50V_52G_53A_54A_55F_67L_ | 3.075534799 |
| 204 | 3914 | TTCGTGTGTGAGATATGTGGGGCACGCTTTactcagaaaggtaacctcctccgccacattaaaCTG | 46F_47V_49E_50I_52G_53A_54R_55F_67L_ | 3.06769753 |
| 205 | 3908 | TTCGTTTGTGAGATCTGTGGGGCAAAATTTactcagaaaggtaacctcctccgccacattaaaCTG | 46F_47V_49E_50I_52G_53A_54K_55F_67L_ | 3.067564926 |
| 206 | 6605 | CTCATCTGTCCTATCTGCGGGTTTGGATGCactcagaaaggtaacctcctccgccacattaaaCTG | 46L_47I_49P_50I_52G_53F_54G_55C_67L_ | 3.067444413 |
| 207 | 3952 | TTCGTGTGTGAGGTGTGTGGGGCACCTTTTactcagaaaggtaacctcctccgccacattaaaCTG | 46F_47V_49E_50V_52G_53A_54P_55F_67L_ | 3.066918408 |
| 208 | 3793 | TTCACCTGTCCAATCTGTGGGGCACAGTTTactcagaaaggtaacctcctccgccacattaaaCTG | 46F_47T_49P_50I_52G_53A_54Q_55F_67L_ | 3.059687361 |
| 209 | 2986 | TTCCAGTGTGAGGTTTGTGGGGCACACTTTactcagaaaggtaacctcctccgccacattaaaCTG | 46F_47Q_49E_50V_52G_53A_54H_55F_67L_ | 3.054777378 |
| 210 | 3700 | TTCACATGTGAAGTGTGTGGGGCAGCTTTTactcagaaaggtaacctcctccgccacattaaaCTG | 46F_47T_49E_50V_52G_53A_54A_55F_67L_ | 3.046839865 |
| 211 | 1780 | TTCATATGTGAGGTCTGTGGGGCAGCGTTTactcagaaaggtaacctcctccgccacattaaaCTG | 46F_47I_49E_50V_52G_53A_54A_55F_67L_ | 3.04322426 |
| 212 | 1754 | TTCATCTGTGAGATTTGTGGGGCACGTTTTactcagaaaggtaacctcctccgccacattaaaCTG | 46F_47I_49E_50I_52G_53A_54R_55F_67L_ | 3.04262375 |
| 213 | 4020 | TTCGTGTGTCCGATCTGTGGGGCAGCGTTTactcagaaaggtaacctcctccgccacattaaaCTG | 46F_47V_49P_50I_52G_53A_54A_55F_67L_ | 3.031551218 |
| 214 | 2974 | TTCCAGTGTGAACAGTGTGGGGCAAGATTTactcagaaaggtaacctcctccgccacattaaaCTG | 46F_47Q_49E_50Q_52G_53A_54R_55F_67L_ | 3.030027565 |
| 215 | 3832 | TTCACTTGTCCCGTCTGTGGGGCACCGTTTactcagaaaggtaacctcctccgccacattaaaCTG | 46F_47T_49P_50V_52G_53A_54P_55F_67L_ | 3.029128035 |
| 216 | 3781 | TTCACGTGTCCGATTTGTGGGGCATGTTTTactcagaaaggtaacctcctccgccacattaaaCTG | 46F_47T_49P_50I_52G_53A_54C_55F_67L_ | 3.0268045 |
| 217 | 2894 | TTCCAATGTGACATTTGTGGGGCACGCTTTactcagaaaggtaacctcctccgccacattaaaCTG | 46F_47Q_49D_50I_52G_53A_54R_55F_67L_ | 3.025754393 |
| 218 | 6760 | CTAACCTGCCCAATCTGCGGGTTCGCTTGTactcagaaaggtaacctcctccgccacattaaaCTG | 46L_47T_49P_50I_52G_53F_54A_55C_67L_ | 3.021480494 |
| 219 | 2961 | TTCCAATGTGAACAGTGTGGGGCATGTTTTactcagaaaggtaacctcctccgccacattaaaCTG | 46F_47Q_49E_50Q_52G_53A_54C_55F_67L_ | 3.018981329 |
| 220 | 3660 | TTCACGTGTGAAATTTGTGGGGCAGCTTTTactcagaaaggtaacctcctccgccacattaaaCTG | 46F_47T_49E_50I_52G_53A_54A_55F_67L_ | 3.017582077 |
| 221 | 7754 | CTGATCTGTGAAATTTGTGGATTCCGGTGCactcagaaaggtaacctcctccgccacattaaaCTG | 46L_47I_49E_50I_52G_53F_54R_55C_67L_ | 3.013688779 |
| 222 | 2937 | TTCCAGTGTGATGTATGTGGGGCAGTTTTTactcagaaaggtaacctcctccgccacattaaaCTG | 46F_47Q_49D_50V_52G_53A_54V_55F_67L_ | 3.013643865 |
| 223 | 2921 | TTCCAGTGTGATGTGTGTGGGGCATGCTTTactcagaaaggtaacctcctccgccacattaaaCTG | 46F_47Q_49D_50V_52G_53A_54C_55F_67L_ | 3.012806061 |
| 224 | 2960 | TTCCAGTGTGAGCAGTGTGGGGCAGCTTTTactcagaaaggtaacctcctccgccacattaaaCTG | 46F_47Q_49E_50Q_52G_53A_54A_55F_67L_ | 3.010689951 |
| 225 | 2880 | TTCCAATGTGATATTTGTGGGGCAGCCTTTactcagaaaggtaacctcctccgccacattaaaCTG | 46F_47Q_49D_50I_52G_53A_54A_55F_67L_ | 3.010260788 |
| 226 | 3794 | TTCACGTGTCCCATATGTGGGGCACGCTTTactcagaaaggtaacctcctccgccacattaaaCTG | 46F_47T_49P_50I_52G_53A_54R_55F_67L_ | 3.001013011 |
| 227 | 908 | TTCGAATGTCCAATCTGTGGGGCAAAGTTTactcagaaaggtaacctcctccgccacattaaaCTG | 46F_47E_49P_50I_52G_53A_54K_55F_67L_ | 3.000469098 |
| 228 | 3675 | TTCACATGTGAAATATGTGGGGCAAGTTTTactcagaaaggtaacctcctccgccacattaaaCTG | 46F_47T_49E_50I_52G_53A_54S_55F_67L_ | 2.99851336 |
| 229 | 3820 | TTCACCTGTCCCGTGTGTGGGGCAGCATTTactcagaaaggtaacctcctccgccacattaaaCTG | 46F_47T_49P_50V_52G_53A_54A_55F_67L_ | 2.99838281 |
| 230 | 2942 | TTCCAGTGTGAGATTTGTGGGGCAGATTTTactcagaaaggtaacctcctccgccacattaaaCTG | 46F_47Q_49E_50I_52G_53A_54D_55F_67L_ | 2.9976386 |
| 231 | 3915 | TTCGTGTGTGAAATCTGTGGGGCAAGCTTTactcagaaaggtaacctcctccgccacattaaaCTG | 46F_47V_49E_50I_52G_53A_54S_55F_67L_ | 2.997354816 |
| 232 | 940 | TTCGAATGTCCTGTATGTGGGGCAGCATTTactcagaaaggtaacctcctccgccacattaaaCTG | 46F_47E_49P_50V_52G_53A_54A_55F_67L_ | 2.996304366 |
| 233 | 7753 | CTAATCTGTGAGATATGCGGCTTTCAGTGCactcagaaaggtaacctcctccgccacattaaaCTG | 46L_47I_49E_50I_52G_53F_54Q_55C_67L_ | 2.995785668 |
| 234 | 6761 | TTAACTTGCCCCATTTGTGGCTTCTGCTGCactcagaaaggtaacctcctccgccacattaaaCTG | 46L_47T_49P_50I_52G_53F_54C_55C_67L_ | 2.995258006 |
| 235 | 3953 | TTCGTTTGTGAAGTGTGTGGGGCACAATTTactcagaaaggtaacctcctccgccacattaaaCTG | 46F_47V_49E_50V_52G_53A_54Q_55F_67L_ | 2.993868591 |
| 236 | 2895 | TTCCAATGTGACATCTGTGGGGCATCATTTactcagaaaggtaacctcctccgccacattaaaCTG | 46F_47Q_49D_50I_52G_53A_54S_55F_67L_ | 2.990760413 |
| 237 | 7875 | CTAAGATGTGAAATCTGCGGCTTTAGTTGCactcagaaaggtaacctcctccgccacattaaaCTG | 46L_47R_49E_50I_52G_53F_54S_55C_67L_ | 2.988529057 |
| 238 | 957 | TTCGAGTGTCCAGTGTGTGGGGCAGTGTTTactcagaaaggtaacctcctccgccacattaaaCTG | 46F_47E_49P_50V_52G_53A_54V_55F_67L_ | 2.987529818 |
| 239 | 3676 | TTCACTTGTGAAATTTGTGGGGCAACCTTTactcagaaaggtaacctcctccgccacattaaaCTG | 46F_47T_49E_50I_52G_53A_54T_55F_67L_ | 2.986824654 |
| 240 | 3094 | TTCCAGTGTCCCCAATGTGGGGCACGCTTTactcagaaaggtaacctcctccgccacattaaaCTG | 46F_47Q_49P_50Q_52G_53A_54R_55F_67L_ | 2.98607864 |
| 241 | 6783 | CTAGTGTGTCCGATCTGTGGCTTTGAATGTactcagaaaggtaacctcctccgccacattaaaCTG | 46L_47V_49P_50I_52G_53F_54E_55C_67L_ | 2.985954087 |
| 242 | 912 | TTCGAGTGTCCTATCTGTGGGGCACCTTTTactcagaaaggtaacctoctccgccacattaaaCTG | 46F_47E_49P_50I_52G_53A_54P_55F_67L_ | 2.984436353 |
| 243 | 4025 | TTCGTCTGTCCAATATGTGGGGCAGGCTTTactcagaaaggtaacctcctccgccacattaaaCTG | 46F_47V_49P_50I_52G_53A_54G_55F_67L_ | 2.983888213 |
| 244 | 7636 | CTTTGCTGTGAGATCTGTGGTTTTACATGTactcagaaaggtaacctcctccgccacattaaaCTG | 46L_47C_49E_50I_52G_53F_54T_55C_67L_ | 2.983496515 |
| 245 | 3119 | TTCCAGTGTCCAGTCTGTGGGGCATATTTTactcagaaaggtaacctcctccgccacattaaaCTG | 46F_47Q_49P_50V_52G_53A_54Y_55F_67L_ | 2.983360176 |
| 246 | 4073 | TTCGTCTGTCCTGTGTGTGGGGCACAGTTTactcagaaaggtaacctcctccgccacattaaaCTG | 46F_47V_49P_50V_52G_53A_54Q_55F_67L_ | 2.981813052 |
| 247 | 2592 | TTCATGTGTCCCATATGTGGGGCACCCTTTactcagaaaggtaacctcctccgccacattaaaCTG | 46F_47M_49P_50I_52G_53A_54P_55F_67L_ | 2.981044702 |
| 248 | 2272 | TTCCTCTGTGAAGTCTGTGGGGCACCCTTTactcagaaaggtaacctcctccgccacattaaaCTG | 46F_47L_49E_50V_52G_53A_54P_55F_67L_ | 2.973762562 |
| 249 | 1873 | TTCATTTGTCCAATTTGTGGGGCACAGTTTactcagaaaggtaacctcctccgccacattaaaCTG | 46F_47I_49P_50I_52G_53A_54Q_55F_67L_ | 2.972227385 |
| 250 | 6781 | CTCGTCTGCCCTATATGCGGCTTCTGCTGCactcagaaaggtaacctcctccgccacattaaaCTG | 46L_47V_49P_50I_52G_53F_54C_55C_67L_ | 2.970012519 |
| 251 | 6660 | CTTATGTGTCCAATCTGCGGTTTTGCATGTactcagaaaggtaacctcctccgccacattaaaCTG | 46L_47M_49P_50I_52G_53F_54A_55C_67L_ | 2.968131258 |
| 252 | 6711 | CTACAATGTCCAATCTGCGGTTTCAATTGTactcagaaaggtaacctcctccgccacattaaaCTG | 46L_47Q_49P_50I_52G_53F_54N_55C_67L_ | 2.966483403 |
| 253 | 6352 | TTCAGGTGTCCAATATGTGGGCGGCCGTTCactcagaaaggtaacctcctccgccacattaaaCTG | 46F_47R_49P_50I_52G_53R_54P_55F_67L_ | 2.966316806 |
| 254 | 7816 | CTCATGTGTGAGATATGTGGGTTTACATGTactcagaaaggtaacctcctccgccacattaaaCTG | 46L_47M_49E_50I_52G_53F_54T_55C_67L_ | 2.966102498 |
| 255 | 2632 | TTCATGTGTCCAGTTTGTGGGGCACCCTTTactcagaaaggtaacctcctccgccacattaaaCTG | 46F_47M_49P_50V_52G_53A_54P_55F_67L_ | 2.963570977 |
| 256 | 2999 | TTCCAGTGTGAGGTGTGTGGGGCATACTTTactcagaaaggtaacctcctccgccacattaaaCTG | 46F_47Q_49E_50V_52G_53A_54Y_55F_67L_ | 2.960494871 |
| 257 | 7928 | CTGGTGTGCGAAATCTGCGGCTTTAAGTGCactcagaaaggtaacctcctccgccacattaaaCTG | 46L_47V_49E_50I_52G_53F_54K_55C_67L_ | 2.950162456 |
| 258 | 1755 | TTCATATGTGAGATTTGTGGGGCATCTTTTactcagaaaggtaacctcctccgccacattaaaCTG | 46F_47I_49E_50I_52G_53A_54S_55F_67L_ | 2.949818227 |
| 259 | 3797 | TTCACCTGTCCTATTTGTGGGGCAGTGTTTactcagaaaggtaacctcctccgccacattaaaCTG | 46F_47T_49P_50I_52G_53A_54V_55F_67L_ | 2.94917833 |
| 260 | 3945 | TTCGTGTGTGAAGTCTGTGGGGCAGGCTTTactcagaaaggtaacctcctccgccacattaaaCTG | 46F_47V_49E_50V_52G_53A_54G_55F_67L_ | 2.94909091 |
| 261 | 3834 | TTCACTTGTCCTGTTTGTGGGGCACGCTTTactcagaaaggtaacctcctccgccacattaaaCTG | 46F_47T_49P_50V_52G_53A_54R_55F_67L_ | 2.947788675 |
| 262 | 1795 | TTCATATGTGAGGTCTGTGGGGCATCGTTTactcagaaaggtaacctcctccgccacattaaaCTG | 46F_47I_49E_50V_52G_53A_54S_55F_67L_ | 2.947306194 |
| 263 | 3087 | TTCCAGTGTCCACAGTGTGGGGCAATCTTTactcagaaaggtaacctcctccgccacattaaaCTG | 46F_47Q_49P_50Q_52G_53A_54I_55F_67L_ | 2.945894916 |
| 264 | 2472 | TTCATGTGTGAGATTTGTGGGGCACCATTTactcagaaaggtaacctcctccgccacattaaaCTG | 46F_47M_49E_50I_52G_53A_54P_55F_67L_ | 2.944977467 |
| 265 | 3839 | TTCACATGTCCTGTGTGTGGGGCATACTTTactcagaaaggtaacctcctccgccacattaaaCTG | 46F_47T_49P_50V_52G_53A_54Y_55F_67L_ | 2.944811205 |
| 266 | 6494 | CTGTGCTGTCCTATATGCGGATTTAGGTGCactcagaaaggtaacctcctccgccacattaaaCTG | 46L_47C_49P_50I_52G_53F_54R_55C_67L_ | 2.94080899 |
| 267 | 6613 | CTGATTTGCCCAATCTGTGGCTTTCAGTGTactcagaaaggtaacctcctccgccacattaaaCTG | 46L_47I_49P_50I_52G_53F_54Q_55C_67L_ | 2.940068571 |
| 268 | 901 | TTCGAGTGTCCAATCTGTGGGGCATGCTTTactcagaaaggtaacctcctccgccacattaaaCTG | 46F_47E_49P_50I_52G_53A_54C_55F_67L_ | 2.93926317 |
| 269 | 954 | TTCGAATGTCCGGTGTGTGGGGCAAGGTTTactcagaaaggtaacctcctccgccacattaaaCTG | 46F_47E_49P_50V_52G_53A_54R_55F_67L_ | 2.938494986 |
| 270 | 6475 | TTGGCGTGTCCAATTTGCGGATTCTCATGTactcagaaaggtaacctcctccgccacattaaaCTG | 46L_47A_49P_50I_52G_53F_54S_55C_67L_ | 2.935313763 |
| 271 | 6759 | CTGTCTTGTCCTATCTGTGGTTTCTATTGCactcagaaaggtaacctcctccgccacattaaaCTG | 46L_47S_49P_50I_52G_53F_54Y_55C_67L_ | 2.933751784 |
| 272 | 2977 | TTCCAGTGTGAGCAGTGTGGGGCAGTCTTTactcagaaaggtaacctcctccgccacattaaaCTG | 46F_47Q_49E_50Q_52G_53A_54V_55F_67L_ | 2.917790818 |
| 273 | 6774 | CTCACCTGCCCTATATGCGGTTTTCGCTGCactcagaaaggtaacctcctccgccacattaaaCTG | 46L_47T_49P_50I_52G_53F_54R_55C_67L_ | 2.917068472 |
| 274 | 3673 | TTCACCTGTGAGATTTGTGGGGCACAATTTactcagaaaggtaacctcctccgccacattaaaCTG | 46F_47T_49E_50I_52G_53A_54Q_55F_67L_ | 2.91678775 |
| 275 | 6332 | TTTCAGTGTCCCATTTGTGGCAGGCCTTTTactcagaaaggtaacctcctccgccacattaaaCTG | 46F_47Q_49P_50I_52G_53R_54P_55F_67L_ | 2.916120685 |
| 276 | 2890 | TTCCAGTGTGATATCTGTGGGGCAATGTTTactcagaaaggtaacctcctccgccacattaaaCTG | 46F_47Q_49D_50I_52G_53A_54M_55F_67L_ | 2.912314327 |
| 277 | 3010 | TTCCAGTGTAATATTTGTGGGGCAATGTTTactcagaaaggtaacctcctccgccacattaaaCTG | 46F_47Q_49N_50I_52G_53A_54M_55F_67L_ | 2.910141506 |
| 278 | 953 | TTCGAGTGTCCCGTTTGTGGGGCACAATTTactcagaaaggtaacctcctccgccacattaaaCTG | 46F_47E_49P_50V_52G_53A_54Q_55F_67L_ | 2.905814685 |
| 279 | 916 | TTCGAGTGTCCCATCTGTGGGGCAACCTTTactcagaaaggtaacctcctccgccacattaaaCTG | 46F_47E_49P_50I_52G_53A_54T_55F_67L_ | 2.902628482 |
| 280 | 955 | TTCGAATGTCCCGTGTGTGGGGCAAGCTTTactcagaaaggtaacctcctccgccacattaaaCTG | 46F_47E_49P_50V_52G_53A_54S_55F_67L_ | 2.902439914 |
| 281 | 354 | TTCTGTTGTGAGGTGTGTGGGGCACGTTTTactcagaaaggtaacctcctccgccacattaaaCTG | 46F_47C_49E_50V_52G_53A_54R_55F_67L_ | 2.899433947 |
| 282 | 2893 | TTCCAGTGTGATATTTGTGGGGCACAGTTTactcagaaaggtaacctcctccgccacattaaaCTG | 46F_47Q_49D_50I_52G_53A_54Q_55F_67L_ | 2.896061887 |
| 283 | 3837 | TTCACGTGTCCAGTGTGTGGGGCAGTCTTTactcagaaaggtaacctcctccgccacattaaaCTG | 46F_47T_49P_50V_52G_53A_54V_55F_67L_ | 2.894584254 |
| 284 | 6479 | CTTGCATGCCCGATCTGCGGCTTTTACTGTactcagaaaggtaacctcctccgccacattaaaCTG | 46L_47A_49P_50I_52G_53F_54Y_55C_67L_ | 2.893270269 |
| 285 | 907 | TTCGAATGTCCCATCTGTGGGGCAATCTTTactcagaaaggtaacctcctccgccacattaaaCTG | 46F_47E_49P_50I_52G_53A_54I_55F_67L_ | 2.891704953 |
| 286 | 6765 | CTCACTTGTCCGATTTGCGGTTTCGGATGTactcagaaaggtaacctcctccgccacattaaaCTG | 46L_47T_49P_50I_52G_53F_54G_55C_67L_ | 2.890549931 |
| 287 | 2935 | TTCCAATGTGATGTGTGTGGGGCAAGTTTTactcagaaaggtaacctcctccgccacattaaaCTG | 46F_47Q_49D_50V_52G_53A_54S_55F_67L_ | 2.886974905 |
| 288 | 3172 | TTCAGATGTGACGTTTGTGGGGCACCGTTTactcagaaaggtaacctcctccgccacattaaaCTG | 46F_47R_49D_50V_52G_53A_54P_55F_67L_ | 2.884531678 |
| 289 | 3796 | TTCACATGTCCAATCTGTGGGGCAACCTTTactcagaaaggtaacctcctccgccacattaaaCTG | 46F_47T_49P_50I_52G_53A_54T_55F_67L_ | 2.883280231 |
| 290 | 3055 | TTCCAATGTAATGTTTGTGGGGCAAGTTTTactcagaaaggtaacctcctccgccacattaaaCTG | 46F_47Q_49N_50V_52G_53A_54S_55F_67L_ | 2.881072591 |
| 291 | 6337 | TTTCAGTGTCCGATCTGTGGTCGAGTCTTTactcagaaaggtaacctcctccgccacattaaaCTG | 46F_47Q_49P_50I_52G_53R_54V_55F_67L_ | 2.87890899 |
| 292 | 6773 | CTGACGTGCCCTATTTGTGGCTTCCAGTGTactcagaaaggtaacctcctccgccacattaaaCTG | 46L_47T_49P_50I_52G_53F_54Q_55C_67L_ | 2.87866054 |
| 293 | 913 | TTCGAGTGTCCTATCTGTGGGGCACAATTTactcagaaaggtaacctcctccgccacattaaaCTG | 46F_47E_49P_50I_52G_53A_54Q_55F_67L_ | 2.877422549 |
| 294 | 2112 | TTCAAGTGTCCTATCTGTGGGGCACCTTTTactcagaaaggtaacctcctccgccacattaaaCTG | 46F_47K_49P_50I_52G_53A_54P_55F_67L_ | 2.874540766 |
| 295 | 6600 | CTAATCTGTCCAATCTGCGGGTTTGCATGCactcagaaaggtaacctcctccgccacattaaaCTG | 46L_47I_49P_50I_52G_53F_54A_55C_67L_ | 2.873298094 |
| 296 | 2927 | TTCCAGTGTGATGTCTGTGGGGCAATATTTactcagaaaggtaacctcctccgccacattaaaCTG | 46F_47Q_49D_50V_52G_53A_54I_55F_67L_ | 2.871956287 |
| 297 | 2928 | TTCCAGTGTGATGTGTGTGGGGCAAAATTTactcagaaaggtaacctcctccgccacattaaaCTG | 46F_47Q_49D_50V_52G_53A_54K_55F_67L_ | 2.869001872 |
| 298 | 3078 | TTCCAGTGTCCTATTTGTGGGGCATGGTTTactcagaaaggtaacctcctccgccacattaaaCTG | 46F_47Q_49P_50I_52G_53A_54W_55F_67L_ | 2.868819011 |
| 299 | 7676 | CTAGAATGCGAGATCTGCGGTTTCACCTGTactcagaaaggtaacctcctccgccacattaaaCTG | 46L_47E_49E_50I_52G_53F_54T_55C_67L_ | 2.866987958 |
| 300 | 919 | TTCGAGTGTCCAATTTGTGGGGCATACTTTactcagaaaggtaacctcctccgccacattaaaCTG | 46F_47E_49P_50I_52G_53A_54Y_55F_67L_ | 2.866787919 |
| 301 | 3661 | TTCACATGTGAAATTTGTGGGGCATGCTTTactcagaaaggtaacctcctccgccacattaaaCTG | 46F_47T_49E_50I_52G_53A_54C_55F_67L_ | 2.857431583 |
| 302 | 6771 | CTCACCTGTCCCATTTGTGGCTTTAACTGTactcagaaaggtaacctcctccgccacattaaaCTG | 46L_47T_49P_50I_52G_53F_54N_55C_67L_ | 2.857146771 |
| 303 | 3679 | TTCACATGTGAAATCTGTGGGGCATACTTTactcagaaaggtaacctcctccgccacattaaaCTG | 46F_47T_49E_50I_52G_53A_54Y_55F_67L_ | 2.85442209 |
| 304 | 3088 | TTCCAGTGTCCTCAATGTGGGGCAAAATTTactcagaaaggtaacctcctccgccacattaaaCTG | 46F_47Q_49P_50Q_52G_53A_54K_55F_67L_ | 2.847402014 |
| 305 | 3791 | TTCACATGTCCTATATGTGGGGCAAATTTTactcagaaaggtaacctcctccgccacattaaaCTG | 46F_47T_49P_50I_52G_53A_54N_55F_67L_ | 2.847381756 |
| 306 | 6799 | CTGGTGTGTCCTATTTGTGGCTTTTACTGCactcagaaaggtaacctcctccgccacattaaaCTG | 46L_47V_49P_50I_52G_53F_54Y_55C_67L_ | 2.841776407 |
| 307 | 6526 | CTGGAGTGTCCAATCTGCGGTTTTCATTGCactcagaaaggtaacctcctccgccacattaaaCTG | 46L_47E_49P_50I_52G_53F_54H_55C_67L_ | 2.841677182 |
| 308 | 6521 | CTGGAGTGTCCCATCTGCGGGTTTTGCTGTactcagaaaggtaacctcctccgccacattaaaCTG | 46L_47E_49P_50I_52G_53F_54C_55C_67L_ | 2.841113658 |
| 309 | 6537 | CTGGAATGTCCTATTTGTGGCTTTGTCTGTactcagaaaggtaacctcctccgccacattaaaCTG | 46L_47E_49P_50I_52G_53F_54V_55C_67L_ | 2.837793042 |
| 310 | 2920 | TTCCAGTGTGACGTCTGTGGGGCAGCCTTTactcagaaaggtaacctcctccgccacattaaaCTG | 46F_47Q_49D_50V_52G_53A_54A_55F_67L_ | 2.836855596 |
| 311 | 6614 | CTGATTTGCCCTATCTGTGGCTTTCGGTGTactcagaaaggtaacctcctccgccacattaaaCTG | 46L_47I_49P_50I_52G_53F_54R_55C_67L_ | 2.835577503 |
| 312 | 2975 | TTCCAATGTGAGCAATGTGGGGCATCATTTactcagaaaggtaacctcctccgccacattaaaCTG | 46F_47Q_49E_50Q_52G_53A_54S_55F_67L_ | 2.835304013 |
| 313 | 3053 | TTCCAGTGTAACGTGTGTGGGGCACAGTTTactcagaaaggtaacctcctccgccacattaaaCTG | 46F_47Q_49N_50V_52G_53A_54Q_55F_67L_ | 2.833151825 |
| 314 | 956 | TTCGAGTGTCCTGTATGTGGGGCAACCTTTactcagaaaggtaacctcctccgccacattaaaCTG | 46F_47E_49P_50V_52G_53A_54T_55F_67L_ | 2.818678606 |
| 315 | 3009 | TTCCAGTGTAACATCTGTGGGGCACTGTTTactcagaaaggtaacctcctccgccacattaaaCTG | 46F_47Q_49N_50I_52G_53A_54L_55F_67L_ | 2.815601244 |
| 316 | 4031 | TTCGTCTGTCCAATCTGTGGGGCAAACTTTactcagaaaggtaacctcctccgccacattaaaCTG | 46F_47V_49P_50I_52G_53A_54N_55F_67L_ | 2.814263034 |
| 317 | 6616 | CTGATCTGTCCCATCTGTGGGTTTACCTGCactcagaaaggtaacctcctccgccacattaaaCTG | 46L_47I_49P_50I_52G_53F_54T_55C_67L_ | 2.812288544 |
| 318 | 3095 | TTCCAATGTCCGCAGTGTGGGGCATCCTTTactcagaaaggtaacctcctccgccacattaaaCTG | 46F_47Q_49P_50Q_52G_53A_54S_55F_67L_ | 2.810654555 |
| 319 | 2934 | TTCCAATGTGATGTCTGTGGGGCACGATTTactcagaaaggtaacctcctccgccacattaaaCTG | 46F_47Q_49D_50V_52G_53A_54R_55F_67L_ | 2.809741736 |
| 320 | 6755 | CTTTCGTGTCCCATTTGCGGTTTCTCATGCactcagaaaggtaacctcctccgccacattaaaCTG | 46L_47S_49P_50I_52G_53F_54S_55C_67L_ | 2.804350847 |
| 321 | 4074 | TTCGTCTGTCCAGTGTGTGGGGCAAGGTTTactcagaaaggtaacctcctccgccacattaaaCTG | 46F_47V_49P_50V_52G_53A_54R_55F_67L_ | 2.803209079 |
| 322 | 4033 | TTCGTGTGTCCGATTTGTGGGGCACAGTTTactcagaaaggtaacctcctccgccacattaaaCTG | 46F_47V_49P_50I_52G_53A_54Q_55F_67L_ | 2.801726897 |
| 323 | 3048 | TTCCAGTGTAACGTTTGTGGGGCAAAATTTactcagaaaggtaacctcctccgccacattaaaCTG | 46F_47Q_49N_50V_52G_53A_54K_55F_67L_ | 2.801355756 |
| 324 | 3831 | TTCACATGTCCCGTGTGTGGGGCAAACTTTactcagaaaggtaacctcctccgccacattaaaCTG | 46F_47T_49P_50V_52G_53A_54N_55F_67L_ | 2.794100863 |
| 325 | 4065 | TTCGTGTGTCCCGTCTGTGGGGCAGGATTTactcagaaaggtaacctcctccgccacattaaaCTG | 46F_47V_49P_50V_52G_53A_54G_55F_67L_ | 2.789980573 |
| 326 | 3835 | TTCACCTGTCCAGTTTGTGGGGCAAGCTTTactcagaaaggtaacctcctccgccacattaaaCTG | 46F_47T_49P_50V_52G_53A_54S_55F_67L_ | 2.788902068 |
| 327 | 7695 | CTTTTCTGCGAAATTTGTGGCTTCTCCTGCactcagaaaggtaacctcctccgccacattaaaCTG | 46L_47F_49E_50I_52G_53F_54S_55C_67L_ | 2.787180228 |
| 328 | 6676 | CTGATGTGTCCCATTTGCGGCTTCACCTGCactcagaaaggtaacctcctccgccacattaaaCTG | 46L_47M_49P_50I_52G_53F_54T_55C_67L_ | 2.786569862 |
| 329 | 4075 | TTCGTCTGTCCTGTGTGTGGGGCATCTTTTactcagaaaggtaacctcctccgccacattaaaCTG | 46F_47V_49P_50V_52G_53A_54S_55F_67L_ | 2.785155336 |
| 330 | 3713 | TTCACCTGTGAGGTGTGTGGGGCACAATTTactcagaaaggtaacctcctccgccacattaaaCTG | 46F_47T_49E_50V_52G_53A_54Q_55F_67L_ | 2.784984687 |
| 331 | 6766 | CTCACATGCCCAATCTGTGGGTTCCACTGTactcagaaaggtaacctcctccgccacattaaaCTG | 46L_47T_49P_50I_52G_53F_54H_55C_67L_ | 2.784658025 |
| 332 | 902 | TTCGAATGTCCCATATGTGGGGCAGATTTTactcagaaaggtaacctcctccgccacattaaaCTG | 46F_47E_49P_50I_52G_53A_54D_55F_67L_ | 2.784503373 |
| 333 | 1860 | TTCATTTGTCCTATATGTGGGGCAGCCTTTactcagaaaggtaacctcctccgccacattaaaCTG | 46F_47I_49P_50I_52G_53A_54A_55F_67L_ | 2.783108592 |
| 334 | 6555 | TTGTTTTGTCCTATATGCGGGTTTTCATGCactcagaaaggtaacctcctccgccacattaaaCTG | 46L_47F_49P_50I_52G_53F_54S_55C_67L_ | 2.781150033 |
| 335 | 6788 | CTGGTCTGCCCCATATGTGGTTTTAAGTGCactcagaaaggtaacctcctccgccacattaaaCTG | 46L_47V_49P_50I_52G_53F_54K_55C_67L_ | 2.778004562 |
| 336 | 6480 | CTGTGTTGCCCCATCTGTGGCTTTGCATGCactcagaaaggtaacctcctccgccacattaaaCTG | 46L_47C_49P_50I_52G_53F_54A_55C_67L_ | 2.773106455 |
| 337 | 3708 | TTCACCTGTGAGGTGTGTGGGGCAAAGTTTactcagaaaggtaacctcctccgccacattaaaCTG | 46F_47T_49E_50V_52G_53A_54K_55F_67L_ | 2.770576989 |
| 338 | 3292 | TTCAGATGTAATGTTTGTGGGGCACCATTTactcagaaaggtaacctcctccgccacattaaaCTG | 46F_47R_49N_50V_52G_53A_54P_55F_67L_ | 2.767747152 |
| 339 | 4071 | TTCGTGTGTCCTGTGTGTGGGGCAAATTTTactcagaaaggtaacctcctccgccacattaaaCTG | 46F_47V_49P_50V_52G_53A_54N_55F_67L_ | 2.767039735 |
| 340 | 1900 | TTCATATGTCCCGTATGTGGGGCAGCCTTTactcagaaaggtaacctcctccgccacattaaaCTG | 46F_47I_49P_50V_52G_53A_54A_55F_67L_ | 2.765555224 |
| 341 | 674 | TTCGACTGTCCTATTTGTGGGGCAAGATTTactcagaaaggtaacctcctccgccacattaaaCTG | 46F_47D_49P_50I_52G_53A_54R_55F_67L_ | 2.762520519 |
| 342 | 1745 | TTCATTTGTGAAATCTGTGGGGCAGGGTTTactcagaaaggtaacctcctccgccacattaaaCTG | 46F_47I_49E_50I_52G_53A_54G_55F_67L_ | 2.760087598 |
| 343 | 1812 | TTCATCTGTAATATTTGTGGGGCACCCTTTactcagaaaggtaacctcctccgccacattaaaCTG | 46F_47I_49N_50I_52G_53A_54P_55F_67L_ | 2.755189809 |
| 344 | 1781 | TTCATCTGTGAAGTGTGTGGGGCATGTTTTactcagaaaggtaacctcctccgccacattaaaCTG | 46F_47I_49E_50V_52G_53A_54C_55F_67L_ | 2.75065235 |
| 345 | 947 | TTCGAGTGTCCTGTTTGTGGGGCAATCTTTactcagaaaggtaacctcctccgccacattaaaCTG | 46F_47E_49P_50V_52G_53A_54I_55F_67L_ | 2.748997263 |
| 346 | 3901 | TTCGTGTGTGAGATATGTGGGGCATGCTTTactcagaaaggtaacctcctccgccacattaaaCTG | 46F_47V_49E_50I_52G_53A_54C_55F_67L_ | 2.747172325 |
| 347 | 941 | TTCGAGTGTCCTGTTTGTGGGGCATGCTTTactcagaaaggtaacctcctccgccacattaaaCTG | 46F_47E_49P_50V_52G_53A_54C_55F_67L_ | 2.746972427 |
| 348 | 7740 | CTTATCTGCGAGATTTGTGGATTTGCGTGTactcagaaaggtaacctcctccgccacattaaaCTG | 46L_47I_49E_50I_52G_53F_54A_55C_67L_ | 2.744431814 |
| 349 | 5972 | TTTAGATGTCCGATTTGCGGACACCCATTTactcagaaaggtaacctcctccgccacattaaaCTG | 46F_47R_49P_50I_52G_53H_54P_55F_67L_ | 2.743790792 |
| 350 | 3056 | TTCCAATGTAACGTGTGTGGGGCAACCTTTactcagaaaggtaacctcctccgccacattaaaCTG | 46F_47Q_49N_50V_52G_53A_54T_55F_67L_ | 2.742747323 |
| 351 | 6791 | CTCGTATGCCCAATATGCGGATTTAACTGTactcagaaaggtaacctcctccgccacattaaaCTG | 46L_47V_49P_50I_52G_53F_54N_55C_67L_ | 2.737243082 |
| 352 | 3951 | TTCGTGTGTGAAGTGTGTGGGGCAAACTTTactcagaaaggtaacctcctccgccacattaaaCTG | 46F_47V_49E_50V_52G_53A_54N_55F_67L_ | 2.736407387 |
| 353 | 3948 | TTCGTGTGTGAAGTGTGTGGGGCAAAATTTactcagaaaggtaacctcctccgccacattaaaCTG | 46F_47V_49E_50V_52G_53A_54K_55F_67L_ | 2.732481568 |
| 354 | 1913 | TTCATCTGTCCTGTGTGTGGGGCACAATTTactcagaaaggtaacctcctccgccacattaaaCTG | 46F_47I_49P_50V_52G_53A_54Q_55F_67L_ | 2.731506007 |
| 355 | 3732 | TTCACATGTAATATCTGTGGGGCACCCTTTactcagaaaggtaacctcctccgccacattaaaCTG | 46F_47T_49N_50I_52G_53A_54P_55F_67L_ | 2.728677903 |
| 356 | 7914 | CTCACCTGTGAAATTTGCGGCTTCCGGTGCactcagaaaggtaacctcctccgccacattaaaCTG | 46L_47T_49E_50I_52G_53F_54R_55C_67L_ | 2.728464798 |
| 357 | 7939 | TTGGTCTGTGAGATTTGTGGCTTTTATTGTactcagaaaggtaacctcctccgccacattaaaCTG | 46L_47V_49E_50I_52G_53F_54Y_55C_67L_ | 2.727491842 |
| 358 | 2967 | TTCCAGTGTGAACAATGTGGGGCAATCTTTactcagaaaggtaacctcctccgccacattaaaCTG | 46F_47Q_49E_50Q_52G_53A_54I_55F_67L_ | 2.722930573 |
| 359 | 3833 | TTCACTTGTCCTGTGTGTGGGGCACAGTTTactcagaaaggtaacctcctccgccacattaaaCTG | 46F_47T_49P_50V_52G_53A_54Q_55F_67L_ | 2.718953166 |
| 360 | 3002 | TTCCAGTGTAACATATGTGGGGCAGACTTTactcagaaaggtaacctcctccgccacattaaaCTG | 46F_47Q_49N_50I_52G_53A_54D_55F_67L_ | 2.716792399 |
| 361 | 2891 | TTCCAGTGTGACATCTGTGGGGCAAACTTTactcagaaaggtaacctcctccgccacattaaaCTG | 46F_47Q_49D_50I_52G_53A_54N_55F_67L_ | 2.715923815 |
| 362 | 7748 | CTGATATGCGAAATCTGCGGGTTTAAATGCactcagaaaggtaacctcctccgccacattaaaCTG | 46L_47I_49E_50I_52G_53F_54K_55C_67L_ | 2.715643834 |
| 363 | 3712 | TTCACATGTGAGGTATGTGGGGCACCTTTTactcagaaaggtaacctcctccgccacattaaaCTG | 46F_47T_49E_50V_52G_53A_54P_55F_67L_ | 2.710273675 |
| 364 | 348 | TTCTGCTGTGAGGTCTGTGGGGCAAAATTTactcagaaaggtaacctcctccgccacattaaaCTG | 46F_47C_49E_50V_52G_53A_54K_55F_67L_ | 2.708964758 |
| 365 | 4034 | TTCGTCTGTCCAATATGTGGGGCACGGTTTactcagaaaggtaacctcctccgccacattaaaCTG | 46F_47V_49P_50I_52G_53A_54R_55F_67L_ | 2.708508362 |
| 366 | 6735 | CTGCGCTGTCCCATATGTGGTTTCAGCTGTactcagaaaggtaacctcctccgccacattaaaCTG | 46L_47R_49P_50I_52G_53F_54S_55C_67L_ | 2.707128357 |
| 367 | 714 | TTCGACTGTCCAGTGTGTGGGGCACGGTTTactcagaaaggtaacctcctccgccacattaaaCTG | 46F_47D_49P_50V_52G_53A_54R_55F_67L_ | 2.706654213 |
| 368 | 2944 | TTCCAGTGTGAAATTTGTGGGGCATTTTTTactcagaaaggtaacctcctccgccacattaaaCTG | 46F_47Q_49E_50I_52G_53A_54F_55F_67L_ | 2.706605138 |
| 369 | 3787 | TTCACGTGTCCAATCTGTGGGGCAATCTTTactcagaaaggtaacctcctccgccacattaaaCTG | 46F_47T_49P_50I_52G_53A_54I_55F_67L_ | 2.705674985 |
| 370 | 3054 | TTCCAGTGTAACGTCTGTGGGGCAAGGTTTactcagaaaggtaacctcctccgccacattaaaCTG | 46F_47Q_49N_50V_52G_53A_54R_55F_67L_ | 2.705541405 |
| 371 | 3668 | TTCACTTGTGAAATCTGTGGGGCAAAGTTTactcagaaaggtaacctcctccgccacattaaaCTG | 46F_47T_49E_50I_52G_53A_54K_55F_67L_ | 2.705164695 |
| 372 | 6764 | CTAACATGCCCCATATGTGGATTCTTCTGCactcagaaaggtaacctcctccgccacattaaaCTG | 46L_47T_49P_50I_52G_53F_54F_55C_67L_ | 2.70349064 |
| 373 | 3941 | TTCGTGTGTGAGGTGTGTGGGGCATGCTTTactcagaaaggtaacctcctccgccacattaaaCTG | 46F_47V_49E_50V_52G_53A_54C_55F_67L_ | 2.702935918 |
| 374 | 6599 | CTTCATTGCCCCATCTGCGGGTTCTACTGCactcagaaaggtaacctcctccgccacattaaaCTG | 46L_47H_49P_50I_52G_53F_54Y_55C_67L_ | 2.702721923 |
| 375 | 2968 | TTCCAGTGTGAGCAGTGTGGGGCAAAGTTTactcagaaaggtaacctcctccgccacattaaaCTG | 46F_47Q_49E_50Q_52G_53A_54K_55F_67L_ | 2.701139551 |
| 376 | 3911 | TTCGTTTGTGAGATTTGTGGGGCAAATTTTactcagaaaggtaacctcctccgccacattaaaCTG | 46F_47V_49E_50I_52G_53A_54N_55F_67L_ | 2.700226031 |
| 377 | 3118 | TTCCAATGTCCCGTTTGTGGGGCATGGTTTactcagaaaggtaacctcctccgccacattaaaCTG | 46F_47Q_49P_50V_52G_53A_54W_55F_67L_ | 2.698430247 |
| 378 | 6835 | TTGTACTGCCCTATATGTGGGTTCAGCTGCactcagaaaggtaacctcctcogccacattaaaCTG | 46L_47Y_49P_50I_52G_53F_54S_55C_67L_ | 2.697479768 |
| 379 | 1793 | TTCATTTGTGAGGTATGTGGGGCACAGTTTactcagaaaggtaacctcctccgccacattaaaCTG | 46F_47I_49E_50V_52G_53A_54Q_55F_67L_ | 2.694281205 |
| 380 | 6556 | CTGTTTTGTCCCATCTGCGGGTTCACATGTactcagaaaggtaacctcctccgccacattaaaCTG | 46L_47F_49P_50I_52G_53F_54T_55C_67L_ | 2.693897761 |
| 381 | 3050 | TTCCAGTGTAATGTGTGTGGGGCAATGTTTactcagaaaggtaacctcctccgccacattaaaCTG | 46F_47Q_49N_50V_52G_53A_54M_55F_67L_ | 2.693818188 |
| 382 | 7843 | TTGCAATGTGAAATTTGCGGTTTTGAATGCactcagaaaggtaacctcctccgccacattaaaCTG | 46L_47Q_49E_50I_52G_53F_54E_55C_67L_ | 2.691384855 |
| 383 | 6675 | CTAATGTGTCCAATTTGCGGTTTCAGCTGTactcagaaaggtaacctcctccgccacattaaaCTG | 46L_47M_49P_50I_52G_53F_54S_55C_67L_ | 2.691113138 |
| 384 | 3052 | TTCCAGTGTAACGTGTGTGGGGCACCATTTactcagaaaggtaacctcctccgccacattaaaCTG | 46F_47Q_49N_50V_52G_53A_54P_55F_67L_ | 2.686649898 |
| 385 | 6128 | TTCGACTGTCCTATCTGCGGTCGAAAGTTTactcagaaaggtaacctcctccgccacattaaaCTG | 46F_47D_49P_50I_52G_53R_54K_55F_67L_ | 2.680407319 |
| 386 | 1788 | TTCATATGTGAGGTGTGTGGGGCAAAATTTactcagaaaggtaacctcctccgccacattaaaCTG | 46F_47I_49E_50V_52G_53A_54K_55F_67L_ | 2.677328109 |
| 387 | 1751 | TTCATCTGTGAAATCTGTGGGGCAAATTTTactcagaaaggtaacctcctccgccacattaaaCTG | 46F_47I_49E_50I_52G_53A_54N_55F_67L_ | 2.675676796 |
| 388 | 3064 | TTCCAATGTCCCATTTGTGGGGCATTCTTTactcagaaaggtaacctcctccgccacattaaaCTG | 46F_47Q_49P_50I_52G_53A_54F_55F_67L_ | 2.671680585 |
| 389 | 3780 | TTCACGTGTCCAATCTGTGGGGCAGCTTTTactcagaaaggtaacctcctccgccacattaaaCTG | 46F_47T_49P_50I_52G_53A_54A_55F_67L_ | 2.670640548 |
| 390 | 3093 | TTCCAGTGTCCACAGTGTGGGGCACAGTTTactcagaaaggtaacctcctccgccacattaaaCTG | 46F_47Q_49P_50Q_52G_53A_54Q_55F_67L_ | 2.668323148 |
| 391 | 432 | TTCTGCTGTCCGATCTGTGGGGCACCTTTTactcagaaaggtaacctcctccgccacattaaaCTG | 46F_47C_49P_50I_52G_53A_54P_55F_67L_ | 2.667623288 |
| 392 | 3701 | TTCACTTGTGAGGTCTGTGGGGCATGTTTTactcagaaaggtaacctcctccgccacattaaaCTG | 46F_47T_49E_50V_52G_53A_54C_55F_67L_ | 2.663606901 |
| 393 | 6134 | TTCGATTGCCCAATTTGCGGACGTCGCTTCactcagaaaggtaacctcctccgccacattaaaCTG | 46F_47D_49P_50I_52G_53R_54R_55F_67L_ | 2.661344182 |
| 394 | 6768 | CTTACTTGTCCAATATGTGGATTTAAGTGTactcagaaaggtaacctcctccgccacattaaaCTG | 46L_47T_49P_50I_52G_53F_54K_55C_67L_ | 2.652976895 |
| 395 | 7634 | CTATGTTGCGAGATCTGTGGCTTCCGCTGTactcagaaaggtaacctcctccgccacattaaaCTG | 46L_47C_49E_50I_52G_53F_54R_55C_67L_ | 2.652835878 |
| 396 | 952 | TTCGAGTGTCCAGTATGTGGGGCACCTTTTactcagaaaggtaacctcctccgccacattaaaCTG | 46F_47E_49P_50V_52G_53A_54P_55F_67L_ | 2.648288843 |
| 397 | 7796 | TTACTGTGTGAAATCTGTGGGTTCACATGCactcagaaaggtaacctcctccgccacattaaaCTG | 46L_47L_49E_50I_52G_53F_54T_55C_67L_ | 2.646765677 |
| 398 | 6656 | CTGTTATGCCCTATCTGCGGCTTTACTTGTactcagaaaggtaacctcctccgccacattaaaCTG | 46L_47L_49P_50I_52G_53F_54T_55C_67L_ | 2.643567152 |
| 399 | 192 | TTCGCTTGTCCCATTTGTGGGGCACCATTTactcagaaaggtaacctcctccgccacattaaaCTG | 46F_47A_49P_50I_52G_53A_54P_55F_67L_ | 2.642992486 |
| 400 | 3677 | TTCACATGTGAAATCTGTGGGGCAGTTTTTactcagaaaggtaacctcctccgccacattaaaCTG | 46F_47T_49E_50I_52G_53A_54V_55F_67L_ | 2.642694517 |
| 401 | 1741 | TTCATCTGTGAGATTTGTGGGGCATGCTTTactcagaaaggtaacctcctccgccacattaaaCTG | 46F_47I_49E_50I_52G_53A_54C_55F_67L_ | 2.637624371 |
| 402 | 6481 | CTGTGCTGTCCGATTTGTGGATTCTGTTGTactcagaaaggtaacctcctccgccacattaaaCTG | 46L_47C_49P_50I_52G_53F_54C_55C_67L_ | 2.634658604 |
| 403 | 3836 | TTCACGTGTCCCGTCTGTGGGGCAACCTTTactcagaaaggtaacctcctccgccacattaaaCTG | 46F_47T_49P_50V_52G_53A_54T_55F_67L_ | 2.633449903 |
| 404 | 6836 | CTCTATTGTCCCATTTGTGGGTTCACTTGCactcagaaaggtaacctcctccgccacattaaaCTG | 46L_47Y_49P_50I_52G_53F_54T_55C_67L_ | 2.631949123 |
| 405 | 6790 | CTTGTATGTCCAATCTGCGGCTTTATGTGCactcagaaaggtaacctcctccgccacattaaaCTG | 46L_47V_49P_50I_52G_53F_54M_55C_67L_ | 2.630017541 |
| 406 | 1871 | TTCATCTGTCCTATCTGTGGGGCAAACTTTactcagaaaggtaacctcctccgccacattaaaCTG | 46F_47I_49P_50I_52G_53A_54N_55F_67L_ | 2.629460804 |
| 407 | 1874 | TTCATCTGTCCCATTTGTGGGGCACGATTTactcagaaaggtaacctcctccgccacattaaaCTG | 46F_47I_49P_50I_52G_53A_54R_55F_67L_ | 2.627800786 |
| 408 | 6603 | CTGATATGTCCTATTTGCGGATTCGAATGTactcagaaaggtaacctcctccgccacattaaaCTG | 46L_47I_49P_50I_52G_53F_54E_55C_67L_ | 2.624843379 |
| 409 | 3220 | TTCCGATGTGAAGTGTGTGGGGCAGCATTTactcagaaaggtaacctcctccgccacattaaaCTG | 46F_47R_49E_50V_52G_53A_54A_55F_67L_ | 2.623345085 |
| 410 | 3552 | TTCTCATGTCCCATCTGTGGGGCACCGTTTactcagaaaggtaacctcctccgccacattaaaCTG | 46F_47S_49P_50I_52G_53A_54P_55F_67L_ | 2.622236144 |
| 411 | 6330 | TTTCAGTGTCCCATATGCGGACGAATGTTCactcagaaaggtaacctcctccgccacattaaaCTG | 46F_47Q_49P_50I_52G_53R_54M_55F_67L_ | 2.619391529 |
| 412 | 788 | TTCGAATGTGAAATATGTGGGGCAAAATTTactcagaaaggtaacctcctccgccacattaaaCTG | 46F_47E_49E_50I_52G_53A_54K_55F_67L_ | 2.619332609 |
| 413 | 1915 | TTCATCTGTCCAGTGTGTGGGGCATCCTTTactcagaaaggtaacctcctccgccacattaaaCTG | 46F_47I_49P_50V_52G_53A_54S_55F_67L_ | 2.619273188 |
| 414 | 3716 | TTCACCTGTGAAGTTTGTGGGGCAACCTTTactcagaaaggtaacctcctccgccacattaaaCTG | 46F_47T_49E_50V_52G_53A_54T_55F_67L_ | 2.617499511 |
| 415 | 948 | TTCGAGTGTCCTGTCTGTGGGGCAAAGTTTactcagaaaggtaacctcctccgccacattaaaCTG | 46F_47E_49P_50V_52G_53A_54K_55F_67L_ | 2.616152397 |
| 416 | 1863 | TTCATATGTCCGATCTGTGGGGCAGAGTTTactcagaaaggtaacctcctccgccacattaaaCTG | 46F_47I_49P_50I_52G_53A_54E_55F_67L_ | 2.613080303 |
| 417 | 3852 | TTCGTCTGTGATATTTGTGGGGCACCATTTactcagaaaggtaacctcctccgccacattaaaCTG | 46F_47V_49D_50I_52G_53A_54P_55F_67L_ | 2.612595443 |
| 418 | 6476 | TTAGCCTGTCCCATTTGCGGGTTTACATGCactcagaaaggtaacctcctccgccacattaaaCTG | 46L_47A_49P_50I_52G_53F_54T_55C_67L_ | 2.612459115 |
| 419 | 1748 | TTCATTTGTGAGATCTGTGGGGCAAAGTTTactcagaaaggtaacctcctccgccacattaaaCTG | 46F_47I_49E_50I_52G_53A_54K_55F_67L_ | 2.611849251 |
| 420 | 1794 | TTCATCTGTGAAGTCTGTGGGGCACGCTTTactcagaaaggtaacctcctccgccacattaaaCTG | 46F_47I_49E_50V_52G_53A_54R_55F_67L_ | 2.604609506 |
| 421 | 2512 | TTCATGTGTGAAGTCTGTGGGGCACCCTTTactcagaaaggtaacctcctccgccacattaaaCTG | 46F_47M_49E_50V_52G_53A_54P_55F_67L_ | 2.602199564 |
| 422 | 7913 | CTCACCTGCGAGATATGCGGGTTTCAGTGCactcagaaaggtaacctcctccgccacattaaaCTG | 46L_47T_49E_50I_52G_53F_54Q_55C_67L_ | 2.600223396 |
| 423 | 1875 | TTCATCTGTCCGATTTGTGGGGCATCCTTTactcagaaaggtaacctcctccgccacattaaaCTG | 46F_47I_49P_50I_52G_53A_54S_55F_67L_ | 2.599137357 |
| 424 | 4023 | TTCGTGTGTCCCATCTGTGGGGCAGAATTTactcagaaaggtaacctcctccgccacattaaaCTG | 46F_47V_49P_50I_52G_53A_54E_55F_67L_ | 2.596630621 |
| 425 | 6756 | CTCAGCTGTCCTATTTGTGGATTTACCTGTactcagaaaggtaacctcctccgccacattaaaCTG | 46L_47S_49P_50I_52G_53F_54T_55C_67L_ | 2.593330997 |
| 426 | 828 | TTCGAATGTGAAGTGTGTGGGGCAAAATTTactcagaaaggtaacctcctccgccacattaaaCTG | 46F_47E_49E_50V_52G_53A_54K_55F_67L_ | 2.592783365 |
| 427 | 4039 | TTCGTCTGTCCTATCTGTGGGGCATATTTTactcagaaaggtaacctcctccgccacattaaaCTG | 46F_47V_49P_50I_52G_53A_54Y_55F_67L_ | 2.591237247 |
| 428 | 3719 | TTCACCTGTGAGGTGTGTGGGGCATATTTTactcagaaaggtaacctcctccgccacattaaaCTG | 46F_47T_49E_50V_52G_53A_54Y_55F_67L_ | 2.585162623 |
| 429 | 6718 | CTGCAGTGCCCCATCTGCGGTTTCTGGTGCactcagaaaggtaacctcctccgccacattaaaCTG | 46L_47Q_49P_50I_52G_53F_54W_55C_67L_ | 2.58505086 |
| 430 | 959 | TTCGAGTGTCCAGTGTGTGGGGCATATTTTactcagaaaggtaacctcctccgccacattaaaCTG | 46F_47E_49P_50V_52G_53A_54Y_55F_67L_ | 2.584242879 |
| 431 | 903 | TTCGAGTGTCCTATCTGTGGGGCAGAATTTactcagaaaggtaacctcctccgccacattaaaCTG | 46F_47E_49P_50I_52G_53A_54E_55F_67L_ | 2.580596312 |
| 432 | 6485 | TTATGCTGTCCCATATGTGGATTCGGCTGCactcagaaaggtaacctcctccgccacattaaaCTG | 46L_47C_49P_50I_52G_53F_54G_55C_67L_ | 2.580428877 |
| 433 | 6154 | TTTGAATGCCCTATCTGTGGCCGCAGATTCactcagaaaggtaacctcctccgccacattaaaCTG | 46F_47E_49P_50I_52G_53R_54R_55F_67L_ | 2.578804354 |
| 434 | 6331 | TTCCAGTGCCCCATTTGCGGTAGAAACTTCactcagaaaggtaacctcctccgccacattaaaCTG | 46F_47Q_49P_50I_52G_53R_54N_55F_67L_ | 2.577655964 |
| 435 | 6720 | CTCAGATGTCCTATATGTGGTTTCGCATGCactcagaaaggtaacctcctccgccacattaaaCTG | 46L_47R_49P_50I_52G_53F_54A_55C_67L_ | 2.573352586 |
| 436 | 6786 | CTGGTGTGCCCTATCTGTGGATTCCATTGCactcagaaaggtaacctcctccgccacattaaaCTG | 46L_47V_49P_50I_52G_53F_54H_55C_67L_ | 2.566542761 |
| 437 | 7859 | CTGCAGTGTGAAATTTGCGGGTTTTACTGCactcagaaaggtaacctcctccgccacattaaaCTG | 46L_47Q_49E_50I_52G_53F_54Y_55C_67L_ | 2.565466476 |
| 438 | 6524 | TTGGAGTGTCCAATCTGTGGCTTCTTTTGTactcagaaaggtaacctcctccgccacattaaaCTG | 46L_47E_49P_50I_52G_53F_54F_55C_67L_ | 2.564161073 |
| 439 | 7696 | CTGTTCTGTGAGATTTGTGGCTTCACGTGCactcagaaaggtaacctcctccgccacattaaaCTG | 46L_47F_49E_50I_52G_53F_54T_55C_67L_ | 2.563627942 |
| 440 | 7635 | CTGTGTTGCGAGATTTGCGGCTTCAGTTGTactcagaaaggtaacctcctccgccacattaaaCTG | 46L_47C_49E_50I_52G_53F_54S_55C_67L_ | 2.561165835 |
| 441 | 673 | TTCGATTGTCCCATCTGTGGGGCACAATTTactcagaaaggtaacctcctccgccacattaaaCTG | 46F_47D_49P_50I_52G_53A_54Q_55F_67L_ | 2.559557707 |
| 442 | 1785 | TTCATCTGTGAGGTGTGTGGGGCAGGCTTTactcagaaaggtaacctcctccgccacattaaaCTG | 46F_47I_49E_50V_52G_53A_54G_55F_67L_ | 2.555544935 |
| 443 | 6514 | TTAGACTGCCCTATATGTGGATTCAGGTGCactcagaaaggtaacctcctccgccacattaaaCTG | 46L_47D_49P_50I_52G_53F_54R_55C_67L_ | 2.554671056 |
| 444 | 3919 | TTCGTGTGTGAGATTTGTGGGGCATACTTTactcagaaaggtaacctcctccgccacattaaaCTG | 46F_47V_49E_50I_52G_53A_54Y_55F_67L_ | 2.553955655 |
| 445 | 906 | TTCGAGTGTCCTATTTGTGGGGCACATTTTactcagaaaggtaacctcctccgccacattaaaCTG | 46F_47E_49P_50I_52G_53A_54H_55F_67L_ | 2.550470689 |
| 446 | 3051 | TTCCAATGTAATGTCTGTGGGGCAAACTTTactcagaaaggtaacctcctccgccacattaaaCTG | 46F_47Q_49N_50V_52G_53A_54N_55F_67L_ | 2.547775645 |
| 447 | 434 | TTCTGCTGTCCCATTTGTGGGGCACGCTTTactcagaaaggtaacctcctccgccacattaaaCTG | 46F_47C_49P_50I_52G_53A_54R_55F_67L_ | 2.546089375 |
| 448 | 6488 | CTCTGCTGCCCTATCTGTGGCTTCAAATGCactcagaaaggtaacctcctccgccacattaaaCTG | 46L_47C_49P_50I_52G_53F_54K_55C_67L_ | 2.54532308 |
| 449 | 3823 | TTCACATGTCCTGTCTGTGGGGCAGAGTTTactcagaaaggtaacctcctccgccacattaaaCTG | 46F_47T_49P_50V_52G_53A_54E_55F_67L_ | 2.544999246 |
| 450 | 3916 | TTCGTGTGTGAGATCTGTGGGGCAACTTTTactcagaaaggtaacctcctccgccacattaaaCTG | 46F_47V_49E_50I_52G_53A_54T_55F_67L_ | 2.544069401 |
| 451 | 6595 | TTACATTGCCCAATATGTGGCTTCTCCTGTactcagaaaggtaacctcctccgccacattaaaCTG | 46L_47H_49P_50I_52G_53F_54S_55C_67L_ | 2.542641164 |
| 452 | 1743 | TTCATCTGTGAGATATGTGGGGCAGAATTTactcagaaaggtaacctcctccgccacattaaaCTG | 46F_47I_49E_50I_52G_53A_54E_55F_67L_ | 2.541099031 |
| 453 | 6740 | CTTAGCTGCCCCATCTGCGGTTTCGCCTGCactcagaaaggtaacctcctccgccacattaaaCTG | 46L_47S_49P_50I_52G_53F_54A_55C_67L_ | 2.538856082 |
| 454 | 4061 | TTCGTCTGTCCAGTGTGTGGGGCATGTTTTactcagaaaggtaacctcctccgccacattaaaCTG | 46F_47V_49P_50V_52G_53A_54C_55F_67L_ | 2.53221892 |
| 455 | 3782 | TTCACTTGTCCTATATGTGGGGCAGACTTTactcagaaaggtaacctcctccgccacattaaaCTG | 46F_47T_49P_50I_52G_53A_54D_55F_67L_ | 2.531993078 |
| 456 | 308 | TTCTGCTGTGAGATCTGTGGGGCAAAGTTTactcagaaaggtaacctcctccgccacattaaaCTG | 46F_47C_49E_50I_52G_53A_54K_55F_67L_ | 2.530107423 |
| 457 | 3090 | TTCCAATGTCCACAGTGTGGGGCAATGTTTactcagaaaggtaacctcctccgccacattaaaCTG | 46F_47Q_49P_50Q_52G_53A_54M_55F_67L_ | 2.529387592 |
| 458 | 4063 | TTCGTCTGTCCGGTGTGTGGGGCAGAGTTTactcagaaaggtaacctcctccgccacattaaaCTG | 46F_47V_49P_50V_52G_53A_54E_55F_67L_ | 2.528375262 |
| 459 | 780 | TTCGAGTGTGAGATCTGTGGGGCAGCGTTTactcagaaaggtaacctcctccgccacattaaaCTG | 46F_47E_49E_50I_52G_53A_54A_55F_67L_ | 2.526339761 |
| 460 | 7668 | CTGGAATGCGAAATCTGCGGATTTAAGTGTactcagaaaggtaacctcctccgccacattaaaCTG | 46L_47E_49E_50I_52G_53F_54K_55C_67L_ | 2.525094497 |
| 461 | 6784 | CTCGTCTGTCCTATCTGCGGATTTTTTTGCactcagaaaggtaacctcctccgccacattaaaCTG | 46L_47V_49P_50I_52G_53F_54F_55C_67L_ | 2.524880587 |
| 462 | 6328 | TTTCAATGCCCAATATGCGGAAGAAAGTTTactcagaaaggtaacctcctccgccacattaaaCTG | 46F_47Q_49P_50I_52G_53R_54K_55F_67L_ | 2.519782022 |
| 463 | 3783 | TTCACCTGTCCCATCTGTGGGGCAGAGTTTactcagaaaggtaacctcctccgccacattaaaCTG | 46F_47T_49P_50I_52G_53A_54E_55F_67L_ | 2.515797982 |
| 464 | 3828 | TTCACATGTCCTGTCTGTGGGGCAAAATTTactcagaaaggtaacctcctccgccacattaaaCTG | 46F_47T_49P_50V_52G_53A_54K_55F_67L_ | 2.512658964 |
| 465 | 6601 | CTGATTTGCCCCATCTGCGGGTTCTGCTGCactcagaaaggtaacctcctccgccacattaaaCTG | 46L_47I_49P_50I_52G_53F_54C_55C_67L_ | 2.511194375 |
| 466 | 820 | TTCGAATGTGAAGTGTGTGGGGCAGCATTTactcagaaaggtaacctcctccgccacattaaaCTG | 46F_47E_49E_50V_52G_53A_54A_55F_67L_ | 2.509994497 |
| 467 | 1992 | TTCAAGTGTGAAATCTGTGGGGCACCCTTTactcagaaaggtaacctcctccgccacattaaaCTG | 46F_47K_49E_50I_52G_53A_54P_55F_67L_ | 2.505783858 |
| 468 | 7849 | CTCCAATGCGAAATCTGTGGGTTCCTGTGCactcagaaaggtaacctcctccgccacattaaaCTG | 46L_47Q_49E_50I_52G_53F_54L_55C_67L_ | 2.50191494 |
| 469 | 793 | TTCGAGTGTGAGATCTGTGGGGCACAGTTTactcagaaaggtaacctcctccgccacattaaaCTG | 46F_47E_49E_50I_52G_53A_54Q_55F_67L_ | 2.501466552 |
| 470 | 3332 | TTCAGATGTCCACAGTGTGGGGCACCATTTactcagaaaggtaacctcctccgccacattaaaCTG | 46F_47R_49P_50Q_52G_53A_54P_55F_67L_ | 2.500730913 |
| 471 | 6655 | CTACTCTGTCCAATTTGCGGTTTTTCATGCactcagaaaggtaacctcctccgccacattaaaCTG | 46L_47L_49P_50I_52G_53F_54S_55C_67L_ | 2.498410006 |
| 472 | 3180 | TTCCGATGTGAGATATGTGGGGCAGCCTTTactcagaaaggtaacctcctccgccacattaaaCTG | 46F_47R_49E_50I_52G_53A_54A_55F_67L_ | 2.493270996 |
| 473 | 2292 | TTCCTGTGTAATATTTGTGGGGCACCTTTTactcagaaaggtaacctcctccgccacattaaaCTG | 46F_47L_49N_50I_52G_53A_54P_55F_67L_ | 2.491294216 |
| 474 | 6531 | CTGGAGTGCCCAATCTGTGGATTCAATTGCactcagaaaggtaacctcctccgccacattaaaCTG | 46L_47E_49P_50I_52G_53F_54N_55C_67L_ | 2.481991029 |
| 475 | 2932 | TTCCAATGTGACGTGTGTGGGGCACCCTTTactcagaaaggtaacctcctccgccacattaaaCTG | 46F_47Q_49D_50V_52G_53A_54P_55F_67L_ | 2.481979837 |
| 476 | 7628 | CTCTGCTGTGAGATCTGTGGGTTTAAATGTactcagaaaggtaacctcctccgccacattaaaCTG | 46L_47C_49E_50I_52G_53F_54K_55C_67L_ | 2.480567609 |
| 477 | 6493 | CTGTGTTGTCCGATCTGCGGCTTTCAGTGCactcagaaaggtaacctcctccgccacattaaaCTG | 46L_47C_49P_50I_52G_53F_54Q_55C_67L_ | 2.479008563 |
| 478 | 7794 | CTGCTTTGTGAGATTTGCGGCTTCCGTTGCactcagaaaggtaacctcctccgccacattaaaCTG | 46L_47L_49E_50I_52G_53F_54R_55C_67L_ | 2.478155004 |
| 479 | 3825 | TTCACCTGTCCGGTTTGTGGGGCAGGTTTTactcagaaaggtaacctcctccgccacattaaaCTG | 46F_47T_49P_50V_52G_53A_54G_55F_67L_ | 2.474843605 |
| 480 | 1692 | TTCATATGTGATATCTGTGGGGCACCATTTactcagaaaggtaacctcctccgccacattaaaCTG | 46F_47I_49D_50I_52G_53A_54P_55F_67L_ | 2.469405775 |
| 481 | 6725 | TTGCGCTGTCCCATATGCGGCTTCGGCTGCactcagaaaggtaacctcctccgccacattaaaCTG | 46L_47R_49P_50I_52G_53F_54G_55C_67L_ | 2.468940125 |
| 482 | 2460 | TTCATGTGTGAGATCTGTGGGGCAGCTTTTactcagaaaggtaacctcctccgccacattaaaCTG | 46F_47M_49E_50I_52G_53A_54A_55F_67L_ | 2.463630278 |
| 483 | 7675 | TTGGAGTGTGAGATTTGCGGGTTTTCCTGCactcagaaaggtaacctcctccgccacattaaaCTG | 46L_47E_49E_50I_52G_53F_54S_55C_67L_ | 2.462819029 |
| 484 | 7900 | CTAACCTGCGAGATATGTGGGTTTGCATGCactcagaaaggtaacctcctccgccacattaaaCTG | 46L_47T_49E_50I_52G_53F_54A_55C_67L_ | 2.462595466 |
| 485 | 356 | TTCTGCTGTGAAGTGTGTGGGGCAACCTTTactcagaaaggtaacctcctccgccacattaaaCTG | 46F_47C_49E_50V_52G_53A_54T_55F_67L_ | 2.459231614 |
| 486 | 300 | TTCTGCTGTGAAATCTGTGGGGCAGCCTTTactcagaaaggtaacctcctccgccacattaaaCTG | 46F_47C_49E_50I_52G_53A_54A_55F_67L_ | 2.458917534 |
| 487 | 3826 | TTCACGTGTCCAGTGTGTGGGGCACACTTTactcagaaaggtaacctcctccgccacattaaaCTG | 46F_47T_49P_50V_52G_53A_54H_55F_67L_ | 2.457825653 |
| 488 | 6335 | TTTCAGTGTCCCATTTGCGGCAGGTCCTTTactcagaaaggtaacctcctccgccacattaaaCTG | 46F_47Q_49P_50I_52G_53R_54S_55F_67L_ | 2.457610437 |
| 489 | 6478 | CTCGCCTGCCCGATTTGCGGGTTCTGGTGCactcagaaaggtaacctcctccgccacattaaaCTG | 46L_47A_49P_50I_52G_53F_54W_55C_67L_ | 2.454853577 |
| 490 | 4028 | TTCGTCTGTCCAATCTGTGGGGCAAAGTTTactcagaaaggtaacctcctccgccacattaaaCTG | 46F_47V_49P_50I_52G_53A_54K_55F_67L_ | 2.447138377 |
| 491 | 7975 | CTCTACTGCGAGATTTGCGGGTTTAGCTGCactcagaaaggtaacctcctccgccacattaaaCTG | 46L_47Y_49E_50I_52G_53F_54S_55C_67L_ | 2.445302047 |
| 492 | 3043 | TTCCAGTGTAATGTATGTGGGGCAGAATTTactcagaaaggtaacctcctccgccacattaaaCTG | 46F_47Q_49N_50V_52G_53A_54E_55F_67L_ | 2.441450693 |
| 493 | 1791 | TTCATTTGTGAGGTGTGTGGGGCAAACTTTactcagaaaggtaacctcctccgccacattaaaCTG | 46F_47I_49E_50V_52G_53A_54N_55F_67L_ | 2.441428937 |
| 494 | 6654 | CTACTGTGTCCTATCTGCGGCTTCAGGTGCactcagaaaggtaacctcctccgccacattaaaCTG | 46L_47L_49P_50I_52G_53F_54R_55C_67L_ | 2.441341334 |
| 495 | 7735 | CTGCACTGTGAAATATGCGGCTTTTCATGCactcagaaaggtaacctcctccgccacattaaaCTG | 46L_47H_49E_50I_52G_53F_54S_55C_67L_ | 2.439862737 |
| 496 | 3005 | TTCCAGTGTAACATATGTGGGGCAGGTTTTactcagaaaggtaacctcctccgccacattaaaCTG | 46F_47Q_49N_50I_52G_53A_54G_55F_67L_ | 2.438659244 |
| 497 | 2883 | TTCCAGTGTGATATCTGTGGGGCAGAGTTTactcagaaaggtaacctcctccgccacattaaaCTG | 46F_47Q_49D_50I_52G_53A_54E_55F_67L_ | 2.437654078 |
| 498 | 4392 | TTCTATTGTGAAATTTGTGGGGCACCATTTactcagaaaggtaacctcctccgccacattaaaCTG | 46F_47Y_49E_50I_52G_53A_54P_55F_67L_ | 2.437135488 |
| 499 | 7921 | CTGGTGTGCGAAATATGTGGCTTCTGTTGCactcagaaaggtaacctcctccgccacattaaaCTG | 46L_47V_49E_50I_52G_53F_54C_55C_67L_ | 2.436540882 |
| 500 | 3784 | TTCACCTGTCCAATCTGTGGGGCATTTTTTactcagaaaggtaacctcctccgccacattaaaCTG | 46F_47T_49P_50I_52G_53A_54F_55F_67L_ | 2.436443887 |
| 501 | 7795 | CTGCTCTGTGAGATCTGTGGGTTCTCCTGTactcagaaaggtaacctcctccgccacattaaaCTG | 46L_47L_49E_50I_52G_53F_54S_55C_67L_ | 2.433663619 |
| 502 | 791 | TTCGAGTGTGAAATTTGTGGGGCAAACTTTactcagaaaggtaacctcctccgccacattaaaCTG | 46F_47E_49E_50I_52G_53A_54N_55F_67L_ | 2.430074522 |
| 503 | 795 | TTCGAATGTGAGATTTGTGGGGCAAGCTTTactcagaaaggtaacctcctccgccacattaaaCTG | 46F_47E_49E_50I_52G_53A_54S_55F_67L_ | 2.429093078 |
| 504 | 2832 | TTCAACTGTCCTATTTGTGGGGCACCTTTTactcagaaaggtaacctcctccgccacattaaaCTG | 46F_47N_49P_50I_52G_53A_54P_55F_67L_ | 2.428485927 |
| 505 | 6336 | TTTCAGTGTCCCATTTGCGGCCGGACATTTactcagaaaggtaacctcctccgccacattaaaCTG | 46F_47Q_49P_50I_52G_53R_54T_55F_67L_ | 2.427000147 |
| 506 | 6460 | CTAGCTTGTCCAATCTGCGGTTTTGCATGTactcagaaaggtaacctcctccgccacattaaaCTG | 46L_47A_49P_50I_52G_53F_54A_55C_67L_ | 2.426159588 |
| 507 | 3671 | TTCACGTGTGAAATCTGTGGGGCAAATTTTactcagaaaggtaacctcctccgccacattaaaCTG | 46F_47T_49E_50I_52G_53A_54N_55F_67L_ | 2.425581283 |
| 508 | 7736 | TTGCATTGCGAAATATGTGGCTTCACTTGTactcagaaaggtaacctcctccgccacattaaaCTG | 46L_47H_49E_50I_52G_53F_54T_55C_67L_ | 2.425187935 |
| 509 | 6724 | CTCCGATGCCCTATCTGTGGTTTTTTTTGCactcagaaaggtaacctcctccgccacattaaaCTG | 46L_47R_49P_50I_52G_53F_54F_55C_67L_ | 2.42432115 |
| 510 | 2532 | TTCATGTGTAATATTTGTGGGGCACCCTTTactcagaaaggtaacctcctccgccacattaaaCTG | 46F_47M_49N_50I_52G_53A_54P_55F_67L_ | 2.42359045 |
| 511 | 3720 | TTCACTTGTAATATCTGTGGGGCAGCCTTTactcagaaaggtaacctcctccgccacattaaaCTG | 46F_47T_49N_50I_52G_53A_54A_55F_67L_ | 2.418704258 |
| 512 | 6516 | CTGGATTGCCCAATTTGCGGCTTCACCTGCactcagaaaggtaacctcctccgccacattaaaCTG | 46L_47D_49P_50I_52G_53F_54T_55C_67L_ | 2.413636103 |
| 513 | 7925 | CTGGTCTGCGAGATCTGCGGCTTTGGCTGCactcagaaaggtaacctcctccgccacattaaaCTG | 46L_47V_49E_50I_52G_53F_54G_55C_67L_ | 2.412648306 |
| 514 | 3734 | TTCACTTGTAATATTTGTGGGGCAAGGTTTactcagaaaggtaacctcctcogccacattaaaCTG | 46F_47T_49N_50I_52G_53A_54R_55F_67L_ | 2.410432222 |
| 515 | 2595 | TTCATGTGTCCTATCTGTGGGGCATCTTTTactcagaaaggtaacctcctccgccacattaaaCTG | 46F_47M_49P_50I_52G_53A_54S_55F_67L_ | 2.410260128 |
| 516 | 4026 | TTCGTGTGTCCTATTTGTGGGGCACACTTTactcagaaaggtaacctcctccgccacattaaaCTG | 46F_47V_49P_50I_52G_53A_54H_55F_67L_ | 2.40979917 |
| 517 | 7814 | TTGATGTGCGAGATCTGTGGGTTTAGATGTactcagaaaggtaacctcctccgccacattaaaCTG | 46L_47M_49E_50I_52G_53F_54R_55C_67L_ | 2.407608397 |
| 518 | 7930 | CTAGTATGTGAGATATGTGGATTTATGTGCactcagaaaggtaacctcctccgccacattaaaCTG | 46L_47V_49E_50I_52G_53F_54M_55C_67L_ | 2.407211236 |
| 519 | 2933 | TTCCAGTGTGACGTGTGTGGGGCACAGTTTactcagaaaggtaacctcctccgccacattaaaCTG | 46F_47Q_49D_50V_52G_53A_54Q_55F_67L_ | 2.405599586 |
| 520 | 6610 | TTAATTTGCCCTATATGTGGTTTTATGTGCactcagaaaggtaacctcctccgccacattaaaCTG | 46L_47I_49P_50I_52G_53F_54M_55C_67L_ | 2.404612653 |
| 521 | 6763 | CTTACTTGTCCTATTTGTGGCTTCGAGTGTactcagaaaggtaacctcctccgccacattaaaCTG | 46L_47T_49P_50I_52G_53F_54E_55C_67L_ | 2.404359418 |
| 522 | 787 | TTCGAGTGTGAAATCTGTGGGGCAATCTTTactcagaaaggtaacctcctccgccacattaaaCTG | 46F_47E_49E_50I_52G_53A_54I_55F_67L_ | 2.404324641 |
| 523 | 6739 | CTCAGATGCCCCATCTGTGGTTTCTATTGCactcagaaaggtaacctcctccgccacattaaaCTG | 46L_47R_49P_50I_52G_53F_54Y_55C_67L_ | 2.403089408 |
| 524 | 3785 | TTCACTTGTCCCATCTGTGGGGCAGGCTTTactcagaaaggtaacctcctccgccacattaaaCTG | 46F_47T_49P_50I_52G_53A_54G_55F_67L_ | 2.399920086 |
| 525 | 3104 | TTCCAGTGTCCCGTTTGTGGGGCATTTTTTactcagaaaggtaacctcctccgccacattaaaCTG | 46F_47Q_49P_50V_52G_53A_54F_55F_67L_ | 2.398472408 |
| 526 | 797 | TTCGAATGTGAGATCTGTGGGGCAGTCTTTactcagaaaggtaacctcctccgccacattaaaCTG | 46F_47E_49E_50I_52G_53A_54V_55F_67L_ | 2.394549599 |
| 527 | 6619 | TTGATCTGTCCAATCTGCGGTTTCTACTGCactcagaaaggtaacctcctccgccacattaaaCTG | 46L_47I_49P_50I_52G_53F_54Y_55C_67L_ | 2.391423281 |
| 528 | 6484 | CTGTGCTGTCCCATCTGTGGGTTTTTCTGCactcagaaaggtaacctcctccgccacattaaaCTG | 46L_47C_49P_50I_52G_53F_54F_55C_67L_ | 2.390856178 |
| 529 | 6744 | CTCTCCTGTCCAATATGTGGCTTTTTTTGCactcagaaaggtaacctcctccgccacattaaaCTG | 46L_47S_49P_50I_52G_53F_54F_55C_67L_ | 2.38883353 |
| 530 | 2152 | TTCAAATGTCCTGTATGTGGGGCACCCTTTactcagaaaggtaacctcctccgccacattaaaCTG | 46F_47K_49P_50V_52G_53A_54P_55F_67L_ | 2.387566757 |
| 531 | 3903 | TTCGTTTGTGAGATCTGTGGGGCAGAGTTTactcagaaaggtaacctcctccgccacattaaaCTG | 46F_47V_49E_50I_52G_53A_54E_55F_67L_ | 2.387345035 |
| 532 | 7908 | CTGACATGCGAGATTTGCGGCTTCAAGTGTactcagaaaggtaacctcctccgccacattaaaCTG | 46L_47T_49E_50I_52G_53F_54K_55C_67L_ | 2.386129747 |
| 533 | 4021 | TTCGTGTGTCCCATCTGTGGGGCATGTTTTactcagaaaggtaacctcctccgccacattaaaCTG | 46F_47V_49P_50I_52G_53A_54C_55F_67L_ | 2.376906588 |
| 534 | 1865 | TTCATCTGTCCCATCTGTGGGGCAGGCTTTactcagaaaggtaacctcctccgccacattaaaCTG | 46F_47I_49P_50I_52G_53A_54G_55F_67L_ | 2.375821178 |
| 535 | 6530 | CTTGAATGTCCTATCTGCGGATTTATGTGCactcagaaaggtaacctcctccgccacattaaaCTG | 46L_47E_49P_50I_52G_53F_54M_55C_67L_ | 2.370945499 |
| 536 | 435 | TTCTGTTGTCCAATTTGTGGGGCATCCTTTactcagaaaggtaacctcctccgccacattaaaCTG | 46F_47C_49P_50I_52G_53A_54S_55F_67L_ | 2.370059356 |
| 537 | 6673 | CTGATGTGCCCAATCTGTGGATTTCAGTGCactcagaaaggtaacctcctccgccacattaaaCTG | 46L_47M_49P_50I_52G_53F_54Q_55C_67L_ | 2.369571807 |
| 538 | 2930 | TTCCAATGTGATGTGTGTGGGGCAATGTTTactcagaaaggtaacctcctccgccacattaaaCTG | 46F_47Q_49D_50V_52G_53A_54M_55F_67L_ | 2.36852739 |
| 539 | 475 | TTCTGTTGTCCAGTCTGTGGGGCATCCTTTactcagaaaggtaacctcctccgccacattaaaCTG | 46F_47C_49P_50V_52G_53A_54S_55F_67L_ | 2.364969878 |
| 540 | 3717 | TTCACTTGTGAAGTGTGTGGGGCAGTCTTTactcagaaaggtaacctcctccgccacattaaaCTG | 46F_47T_49E_50V_52G_53A_54V_55F_67L_ | 2.364298665 |
| 541 | 905 | TTCGAGTGTCCCATATGTGGGGCAGGCTTTactcagaaaggtaacctcctccgccacattaaaCTG | 46F_47E_49P_50I_52G_53A_54G_55F_67L_ | 2.362424189 |
| 542 | 7815 | TTGATGTGTGAAATTTGCGGCTTTAGCTGCactcagaaaggtaacctcctccgccacattaaaCTG | 46L_47M_49E_50I_52G_53F_54S_55C_67L_ | 2.361960362 |
| 543 | 2889 | TTCCAGTGTGACATCTGTGGGGCATTGTTTactcagaaaggtaacctcctccgccacattaaaCTG | 46F_47Q_49D_50I_52G_53A_54L_55F_67L_ | 2.357149953 |
| 544 | 6334 | TTCCAATGCCCAATTTGTGGGCGCAGGTTCactcagaaaggtaacctcctccgccacattaaaCTG | 46F_47Q_49P_50I_52G_53R_54R_55F_67L_ | 2.355643592 |
| 545 | 340 | TTCTGTTGTGAAGTGTGTGGGGCAGCCTTTactcagaaaggtaacctcctccgccacattaaaCTG | 46F_47C_49E_50V_52G_53A_54A_55F_67L_ | 2.350519056 |
| 546 | 1911 | TTCATCTGTCCAGTGTGTGGGGCAAATTTTactcagaaaggtaacctcctccgccacattaaaCTG | 46F_47I_49P_50V_52G_53A_54N_55F_67L_ | 2.345836985 |
| 547 | 3788 | TTCACCTGTCCAATCTGTGGGGCAAAGTTTactcagaaaggtaacctcctccgccacattaaaCTG | 46F_47T_49P_50I_52G_53A_54K_55F_67L_ | 2.345761505 |
| 548 | 315 | TTCTGCTGTGAGATCTGTGGGGCATCATTTactcagaaaggtaacctcctccgccacattaaaCTG | 46F_47C_49E_50I_52G_53A_54S_55F_67L_ | 2.345373985 |
| 549 | 781 | TTCGAGTGTGAAATCTGTGGGGCATGCTTTactcagaaaggtaacctcctccgccacattaaaCTG | 46F_47E_49E_50I_52G_53A_54C_55F_67L_ | 2.345276325 |
| 550 | 6596 | CTGCACTGCCCTATCTGTGGCTTCACATGTactcagaaaggtaacctcctccgccacattaaaCTG | 46L_47H_49P_50I_52G_53F_54T_55C_67L_ | 2.343842446 |
| 551 | 7860 | CTTCGTTGTGAAATCTGTGGCTTCGCCTGCactcagaaaggtaacctcctccgccacattaaaCTG | 46L_47R_49E_50I_52G_53F_54A_55C_67L_ | 2.343474712 |
| 552 | 355 | TTCTGTTGTGAGGTCTGTGGGGCAAGTTTTactcagaaaggtaacctcctccgccacattaaaCTG | 46F_47C_49E_50V_52G_53A_54S_55F_67L_ | 2.343333196 |
| 553 | 3786 | TTCACCTGTCCTATCTGTGGGGCACACTTTactcagaaaggtaacctcctccgccacattaaaCTG | 46F_47T_49P_50I_52G_53A_54H_55F_67L_ | 2.341297201 |
| 554 | 3917 | TTCGTATGTGAGATCTGTGGGGCAGTGTTTactcagaaaggtaacctcctccgccacattaaaCTG | 46F_47V_49E_50I_52G_53A_54V_55F_67L_ | 2.334706151 |
| 555 | 6815 | CTGTGGTGCCCGATCTGTGGATTTAGCTGCactcagaaaggtaacctcctccgccacattaaaCTG | 46L_47W_49P_50I_52G_53F_54S_55C_67L_ | 2.33317937 |
| 556 | 2234 | TTCTTGTGTGAGATCTGTGGGGCAAGGTTTactcagaaaggtaacctcctccgccacattaaaCTG | 46F_47L_49E_50I_52G_53A_54R_55F_67L_ | 2.332603814 |
| 557 | 6333 | TTCCAGTGCCCCATCTGTGGCAGACAGTTCactcagaaaggtaacctcctccgccacattaaaCTG | 46F_47Q_49P_50I_52G_53R_54Q_55F_67L_ | 2.329797202 |
| 558 | 316 | TTCTGCTGTGAGATCTGTGGGGCAACCTTTactcagaaaggtaacctcctccgccacattaaaCTG | 46F_47C_49E_50I_52G_53A_54T_55F_67L_ | 2.329336066 |
| 559 | 6339 | TTCCAATGTCCCATATGTGGCAGGTACTTCactcagaaaggtaacctcctccgccacattaaaCTG | 46F_47Q_49P_50I_52G_53R_54Y_55F_67L_ | 2.328287143 |
| 560 | 854 | TTCGAGTGTAATATTTGTGGGGCAAGATTTactcagaaaggtaacctcctccgccacattaaaCTG | 46F_47E_49N_50I_52G_53A_54R_55F_67L_ | 2.325283707 |
| 561 | 1783 | TTCATCTGTGAAGTATGTGGGGCAGAGTTTactcagaaaggtaacctcctccgccacattaaaCTG | 46F_47I_49E_50V_52G_53A_54E_55F_67L_ | 2.324147143 |
| 562 | 7876 | CTCAGATGCGAAATCTGTGGGTTCACATGTactcagaaaggtaacctcctccgccacattaaaCTG | 46L_47R_49E_50I_52G_53F_54T_55C_67L_ | 2.321387752 |
| 563 | 3705 | TTCACTTGTGAGGTGTGTGGGGCAGGTTTTactcagaaaggtaacctcctccgccacattaaaCTG | 46F_47T_49E_50V_52G_53A_54G_55F_67L_ | 2.320541837 |
| 564 | 6726 | TTGAGATGTCCAATTTGTGGGTTCCACTGTactcagaaaggtaacctcctccgccacattaaaCTG | 46L_47R_49P_50I_52G_53F_54H_55C_67L_ | 2.319783933 |
| 565 | 4079 | TTCGTGTGTCCCGTATGTGGGGCATATTTTactcagaaaggtaacctcctccgccacattaaaCTG | 46F_47V_49P_50V_52G_53A_54Y_55F_67L_ | 2.318931082 |
| 566 | 2580 | TTCATGTGTCCAATTTGTGGGGCAGCCTTTactcagaaaggtaacctcctccgccacattaaaCTG | 46F_47M_49P_50I_52G_53A_54A_55F_67L_ | 2.317272871 |
| 567 | 835 | TTCGAATGTGAAGTGTGTGGGGCATCTTTTactcagaaaggtaacctcctccgccacattaaaCTG | 46F_47E_49E_50V_52G_53A_54S_55F_67L_ | 2.315710989 |
| 568 | 3006 | TTCCAGTGTAATATTTGTGGGGCACATTTTactcagaaaggtaacctcctccgccacattaaaCTG | 46F_47Q_49N_50I_52G_53A_54H_55F_67L_ | 2.315439482 |
| 569 | 7694 | CTTTTCTGTGAAATTTGCGGTTTCAGGTGTactcagaaaggtaacctcctccgccacattaaaCTG | 46L_47F_49E_50I_52G_53F_54R_55C_67L_ | 2.314192927 |
| 570 | 6580 | CTGCACTGCCCAATCTGTGGATTCGCGTGCactcagaaaggtaacctcctccgccacattaaaCTG | 46L_47H_49P_50I_52G_53F_54A_55C_67L_ | 2.313504905 |
| 571 | 831 | TTCGAGTGTGAAGTGTGTGGGGCAAACTTTactcagaaaggtaacctcctccgccacattaaaCTG | 46F_47E_49E_50V_52G_53A_54N_55F_67L_ | 2.312827125 |
| 572 | 6608 | CTCATCTGTCCAATCTGCGGCTTCAAGTGTactcagaaaggtaacctcctccgccacattaaaCTG | 46L_47I_49P_50I_52G_53F_54K_55C_67L_ | 2.312436018 |
| 573 | 3960 | TTCGTGTGTAACATCTGTGGGGCAGCGTTTactcagaaaggtaacctcctccgccacattaaaCTG | 46F_47V_49N_50I_52G_53A_54A_55F_67L_ | 2.31091301 |
| 574 | 3340 | TTCAGGTGTCCAGTATGTGGGGCAGCTTTTactcagaaaggtaacctcctccgccacattaaaCTG | 46F_47R_49P_50V_52G_53A_54A_55F_67L_ | 2.308882256 |
| 575 | 837 | TTCGAGTGTGAGGTCTGTGGGGCAGTGTTTactcagaaaggtaacctcctccgccacattaaaCTG | 46F_47E_49E_50V_52G_53A_54V_55F_67L_ | 2.308729158 |
| 576 | 2970 | TTCCAGTGTGAACAGTGTGGGGCAATGTTTactcagaaaggtaacctcctccgccacattaaaCTG | 46F_47Q_49E_50Q_52G_53A_54M_55F_67L_ | 2.307789441 |
| 577 | 6770 | CTCACCTGTCCTATTTGTGGGTTCATGTGCactcagaaaggtaacctcctccgccacattaaaCTG | 46L_47T_49P_50I_52G_53F_54M_55C_67L_ | 2.304429541 |
| 578 | 6490 | CTATGCTGCCCAATATGCGGGTTTATGTGCactcagaaaggtaacctcctccgccacattaaaCTG | 46L_47C_49P_50I_52G_53F_54M_55C_67L_ | 2.302308444 |
| 579 | 6778 | CTGACATGTCCAATATGCGGTTTCTGGTGCactcagaaaggtaacctcctccgccacattaaaCTG | 46L_47T_49P_50I_52G_53F_54W_55C_67L_ | 2.299325194 |
| 580 | 2973 | TTCCAGTGTGAACAGTGTGGGGCACAATTTactcagaaaggtaacctcctccgccacattaaaCTG | 46F_47Q_49E_50Q_52G_53A_54Q_55F_67L_ | 2.296568411 |
| 581 | 473 | TTCTGCTGTCCCGTTTGTGGGGCACAATTTactcagaaaggtaacctcctccgccacattaaaCTG | 46F_47C_49P_50V_52G_53A_54Q_55F_67L_ | 2.295761978 |
| 582 | 6321 | TTCCAATGTCCCATTTGCGGTAGGTGCTTCactcagaaaggtaacctcctccgccacattaaaCTG | 46F_47Q_49P_50I_52G_53R_54C_55F_67L_ | 2.291917564 |
| 583 | 833 | TTCGAATGTGAGGTCTGTGGGGCACAGTTTactcagaaaggtaacctcctccgccacattaaaCTG | 46F_47E_49E_50V_52G_53A_54Q_55F_67L_ | 2.290928064 |
| 584 | 6329 | TTTCAGTGCCCAATTTGTGGGAGGCTCTTCactcagaaaggtaacctcctccgccacattaaaCTG | 46F_47Q_49P_50I_52G_53R_54L_55F_67L_ | 2.290833419 |
| 585 | 3838 | TTCACCTGTCCAGTTTGTGGGGCATGGTTTactcagaaaggtaacctcctccgccacattaaaCTG | 46F_47T_49P_50V_52G_53A_54W_55F_67L_ | 2.288594559 |
| 586 | 6665 | CTTATGTGTCCAATTTGTGGTTTCGGATGCactcagaaaggtaacctcctccgccacattaaaCTG | 46L_47M_49P_50I_52G_53F_54G_55C_67L_ | 2.285783194 |
| 587 | 6746 | CTATCTTGCCCTATATGCGGCTTTCACTGCactcagaaaggtaacctcctccgccacattaaaCTG | 46L_47S_49P_50I_52G_53F_54H_55C_67L_ | 2.284795361 |
| 588 | 6148 | TTCGAATGCCCAATATGCGGCAGAAAATTCactcagaaaggtaacctcctccgccacattaaaCTG | 46F_47E_49P_50I_52G_53R_54K_55F_67L_ | 2.284047707 |
| 589 | 1632 | TTCCACTGTCCAATTTGTGGGGCACCTTTTactcagaaaggtaacctcctccgccacattaaaCTG | 46F_47H_49P_50I_52G_53A_54P_55F_67L_ | 2.281620899 |
| 590 | 3959 | TTCGTCTGTGAGGTCTGTGGGGCATACTTTactcagaaaggtaacctcctccgccacattaaaCTG | 46F_47V_49E_50V_52G_53A_54Y_55F_67L_ | 2.280959956 |
| 591 | 2500 | TTCATGTGTGAGGTATGTGGGGCAGCTTTTactcagaaaggtaacctcctccgccacattaaaCTG | 46F_47M_49E_50V_52G_53A_54A_55F_67L_ | 2.28012589 |
| 592 | 6013 | TTCACTTGTCCAATCTGCGGTCATCAATTCactcagaaaggtaacctcctccgccacattaaaCTG | 46F_47T_49P_50I_52G_53H_54Q_55F_67L_ | 2.27860974 |
| 593 | 6519 | CTGGACTGCCCCATCTGCGGCTTCTACTGCactcagaaaggtaacctcctccgccacattaaaCTG | 46L_47D_49P_50I_52G_53F_54Y_55C_67L_ | 2.27785514 |
| 594 | 3798 | TTCACATGTCCAATTTGTGGGGCATGGTTTactcagaaaggtaacctcctccgccacattaaaCTG | 46F_47T_49P_50I_52G_53A_54W_55F_67L_ | 2.277725408 |
| 595 | 6525 | TTGGAGTGCCCTATCTGCGGTTTCGGATGCactcagaaaggtaacctcctccgccacattaaaCTG | 46L_47E_49P_50I_52G_53F_54G_55C_67L_ | 2.274934723 |
| 596 | 6653 | CTTCTGTGCCCGATTTGCGGCTTCCAATGTactcagaaaggtaacctcctccgccacattaaaCTG | 46L_47L_49P_50I_52G_53F_54Q_55C_67L_ | 2.274025 |
| 597 | 313 | TTCTGTTGTGAGATATGTGGGGCACAATTTactcagaaaggtaacctcctccgccacattaaaCTG | 46F_47C_49E_50I_52G_53A_54Q_55F_67L_ | 2.273393638 |
| 598 | 3665 | TTCACGTGTGAGATATGTGGGGCAGGATTTactcagaaaggtaacctcctccgccacattaaaCTG | 46F_47T_49E_50I_52G_53A_54G_55F_67L_ | 2.272598433 |
| 599 | 6754 | CTAAGCTGCCCAATATGCGGGTTCCGGTGTactcagaaaggtaacctcctccgccacattaaaCTG | 46L_47S_49P_50I_52G_53F_54R_55C_67L_ | 2.27255619 |
| 600 | 554 | TTCGATTGTGAGATTTGTGGGGCACGGTTTactcagaaaggtaacctcctccgccacattaaaCTG | 46F_47D_49E_50I_52G_53A_54R_55F_67L_ | 2.268255148 |
| 601 | 3943 | TTCGTCTGTGAGGTTTGTGGGGCAGAGTTTactcagaaaggtaacctcctccgccacattaaaCTG | 46F_47V_49E_50V_52G_53A_54E_55F_67L_ | 2.265146254 |
| 602 | 910 | TTCGAGTGTCCAATTTGTGGGGCAATGTTTactcagaaaggtaacctcctccgccacattaaaCTG | 46F_47E_49P_50I_52G_53A_54M_55F_67L_ | 2.264118098 |
| 603 | 6541 | CTGTTCTGCCCCATATGTGGGTTCTGTTGTactcagaaaggtaacctcctccgccacattaaaCTG | 46L_47F_49P_50I_52G_53F_54C_55C_67L_ | 2.263410187 |
| 604 | 474 | TTCTGCTGTCCCGTTTGTGGGGCAAGATTTactcagaaaggtaacctcctccgccacattaaaCTG | 46F_47C_49P_50V_52G_53A_54R_55F_67L_ | 2.262505844 |
| 605 | 2958 | TTCCAGTGTGAGATATGTGGGGCATGGTTTactcagaaaggtaacctcctccgccacattaaaCTG | 46F_47Q_49E_50I_52G_53A_54W_55F_67L_ | 2.261458899 |
| 606 | 341 | TTCTGCTGTGAAGTATGTGGGGCATGTTTTactcagaaaggtaacctcctccgccacattaaaCTG | 46F_47C_49E_50V_52G_53A_54C_55F_67L_ | 2.2560768 |
| 607 | 6486 | CTGTGCTGCCCGATCTGTGGGTTTCATTGCactcagaaaggtaacctcctccgccacattaaaCTG | 46L_47C_49P_50I_52G_53F_54H_55C_67L_ | 2.254177915 |
| 608 | 2984 | TTCCAGTGTGAAGTATGTGGGGCATTTTTTactcagaaaggtaacctcctccgccacattaaaCTG | 46F_47Q_49E_50V_52G_53A_54F_55F_67L_ | 2.252090287 |
| 609 | 6527 | CTGGAATGCCCTATATGTGGATTTATCTGCactcagaaaggtaacctcctccgccacattaaaCTG | 46L_47E_49P_50I_52G_53F_54I_55C_67L_ | 2.251454869 |
| 610 | 6498 | TTGTGCTGCCCAATCTGCGGCTTCTGGTGTactcagaaaggtaacctcctccgccacattaaaCTG | 46L_47C_49P_50I_52G_53F_54W_55C_67L_ | 2.248582337 |
| 611 | 2872 | TTCAATTGTCCAGTGTGTGGGGCACCCTTTactcagaaaggtaacctcctccgccacattaaaCTG | 46F_47N_49P_50V_52G_53A_54P_55F_67L_ | 2.248331771 |
| 612 | 7976 | CTCTATTGCGAAATATGCGGTTTCACATGCactcagaaaggtaacctcctccgccacattaaaCTG | 46L_47Y_49E_50I_52G_53F_54T_55C_67L_ | 2.246273467 |
| 613 | 6464 | TTGGCGTGTCCCATCTGTGGCTTTTTTTGCactcagaaaggtaacctcctccgccacattaaaCTG | 46L_47A_49P_50I_52G_53F_54F_55C_67L_ | 2.244277033 |
| 614 | 3956 | TTCGTCTGTGAAGTCTGTGGGGCAACCTTTactcagaaaggtaacctcctccgccacattaaaCTG | 46F_47V_49E_50V_52G_53A_54T_55F_67L_ | 2.242665285 |
| 615 | 942 | TTCGAATGTCCTGTGTGTGGGGCAGACTTTactcagaaaggtaacctcctccgccacattaaaCTG | 46F_47E_49P_50V_52G_53A_54D_55F_67L_ | 2.241714527 |
| 616 | 6777 | CTCACGTGCCCAATTTGTGGCTTCGTTTGTactcagaaaggtaacctcctccgccacattaaaCTG | 46L_47T_49P_50I_52G_53F_54V_55C_67L_ | 2.239329054 |
| 617 | 301 | TTCTGCTGTGAAATCTGTGGGGCATGTTTTactcagaaaggtaacctcctccgccacattaaaCTG | 46F_47C_49E_50I_52G_53A_54C_55F_67L_ | 2.238208049 |
| 618 | 713 | TTCGACTGTCCCGTGTGTGGGGCACAGTTTactcagaaaggtaacctcctccgccacattaaaCTG | 46F_47D_49P_50V_52G_53A_54Q_55F_67L_ | 2.233216577 |
| 619 | 232 | TTCGCTTGTCCTGTGTGTGGGGCACCCTTTactcagaaaggtaacctcctccgccacattaaaCTG | 46F_47A_49P_50V_52G_53A_54P_55F_67L_ | 2.232343914 |
| 620 | 6584 | CTGCACTGCCCAATATGTGGCTTCTTTTGTactcagaaaggtaacctcctccgccacattaaaCTG | 46L_47H_49P_50I_52G_53F_54F_55C_67L_ | 2.232104864 |
| 621 | 1756 | TTCATCTGTGAAATCTGTGGGGCAACTTTTactcagaaaggtaacctcctccgccacattaaaCTG | 46F_47I_49E_50I_52G_53A_54T_55F_67L_ | 2.230332352 |
| 622 | 4036 | TTCGTGTGTCCAATTTGTGGGGCAACATTTactcagaaaggtaacctcctccgccacattaaaCTG | 46F_47V_49P_50I_52G_53A_54T_55F_67L_ | 2.227580741 |
| 623 | 6736 | CTAAGATGTCCCATCTGTGGATTTACTTGTactcagaaaggtaacctcctccgccacattaaaCTG | 46L_47R_49P_50I_52G_53F_54T_55C_67L_ | 2.226337441 |
| 624 | 431 | TTCTGTTGTCCCATCTGTGGGGCAAATTTTactcagaaaggtaacctcctccgccacattaaaCTG | 46F_47C_49P_50I_52G_53A_54N_55F_67L_ | 2.220692734 |
| 625 | 851 | TTCGAGTGTAATATTTGTGGGGCAAACTTTactcagaaaggtaacctcctccgccacattaaaCTG | 46F_47E_49N_50I_52G_53A_54N_55F_67L_ | 2.218714812 |
| 626 | 6324 | TTTCAATGTCCAATCTGCGGGCGCTTCTTCactcagaaaggtaacctcctccgccacattaaaCTG | 46F_47Q_49P_50I_52G_53R_54F_55F_67L_ | 2.218605092 |
| 627 | 420 | TTCTGCTGTCCTATCTGTGGGGCAGCCTTTactcagaaaggtaacctcctccgccacattaaaCTG | 46F_47C_49P_50I_52G_53A_54A_55F_67L_ | 2.209460584 |
| 628 | 6323 | TTTCAGTGCCCGATTTGTGGGCGGGAATTTactcagaaaggtaacctcctccgccacattaaaCTG | 46F_47Q_49P_50I_52G_53R_54E_55F_67L_ | 2.200678412 |
| 629 | 6483 | CTGTGTTGCCCGATATGCGGTTTTGAATGCactcagaaaggtaacctcctccgccacattaaaCTG | 46L_47C_49P_50I_52G_53F_54E_55C_67L_ | 2.199139042 |
| 630 | 660 | TTCGACTGTCCTATTTGTGGGGCAGCCTTTactcagaaaggtaacctcctccgccacattaaaCTG | 46F_47D_49P_50I_52G_53A_54A_55F_67L_ | 2.199053473 |
| 631 | 4512 | TTCTATTGTCCAATATGTGGGGCACCGTTTactcagaaaggtaacctcctccgccacattaaaCTG | 46F_47Y_49P_50I_52G_53A_54P_55F_67L_ | 2.195719813 |
| 632 | 1074 | TTCTTTTGTGAGGTATGTGGGGCAAGGTTTactcagaaaggtaacctcctccgccacattaaaCTG | 46F_47F_49E_50V_52G_53A_54R_55F_67L_ | 2.192263305 |
| 633 | 3300 | TTCCGCTGTCCAATCTGTGGGGCAGCCTTTactcagaaaggtaacctcctccgccacattaaaCTG | 46F_47R_49P_50I_52G_53A_54A_55F_67L_ | 2.190755124 |
| 634 | 6645 | CTGCTGTGCCCTATCTGCGGCTTTGGCTGCactcagaaaggtaacctcctccgccacattaaaCTG | 46L_47L_49P_50I_52G_53F_54G_55C_67L_ | 2.189836413 |
| 635 | 6679 | CTTATGTGCCCCATTTGCGGATTTTACTGCactcagaaaggtaacctcctccgccacattaaaCTG | 46L_47M_49P_50I_52G_53F_54Y_55C_67L_ | 2.188504534 |
| 636 | 7750 | TTGATCTGTGAGATCTGCGGGTTCATGTGTactcagaaaggtaacctcctccgccacattaaaCTG | 46L_47I_49E_50I_52G_53F_54M_55C_67L_ | 2.181873289 |
| 637 | 821 | TTCGAGTGTGAAGTGTGTGGGGCATGTTTTactcagaaaggtaacctcctccgccacattaaaCTG | 46F_47E_49E_50V_52G_53A_54C_55F_67L_ | 2.180429124 |
| 638 | 3790 | TTCACATGTCCAATCTGTGGGGCAATGTTTactcagaaaggtaacctcctccgccacattaaaCTG | 46F_47T_49P_50I_52G_53A_54M_55F_67L_ | 2.174704173 |
| 639 | 1861 | TTCATCTGTCCCATCTGTGGGGCATGTTTTactcagaaaggtaacctcctccgccacattaaaCTG | 46F_47I_49P_50I_52G_53A_54C_55F_67L_ | 2.172538954 |
| 640 | 4037 | TTCGTGTGTCCTATCTGTGGGGCAGTCTTTactcagaaaggtaacctcctccgccacattaaaCTG | 46F_47V_49P_50I_52G_53A_54V_55F_67L_ | 2.172319262 |
| 641 | 668 | TTCGATTGTCCAATATGTGGGGCAAAATTTactcagaaaggtaacctcctccgccacattaaaCTG | 46F_47D_49P_50I_52G_53A_54K_55F_67L_ | 2.169153956 |
| 642 | 6801 | CTCTGGTGTCCCATTTGTGGCTTTTGCTGCactcagaaaggtaacctcctccgccacattaaaCTG | 46L_47W_49P_50I_52G_53F_54C_55C_67L_ | 2.168694958 |
| 643 | 796 | TTCGAGTGTGAAATCTGTGGGGCAACCTTTactcagaaaggtaacctcctccgccacattaaaCTG | 46F_47E_49E_50I_52G_53A_54T_55F_67L_ | 2.167951819 |
| 644 | 836 | TTCGAATGTGAAGTCTGTGGGGCAACTTTTactcagaaaggtaacctcctccgccacattaaaCTG | 46F_47E_49E_50V_52G_53A_54T_55F_67L_ | 2.165277323 |
| 645 | 6515 | CTGGATTGCCCCATCTGTGGCTTTTCCTGTactcagaaaggtaacctcctccgccacattaaaCTG | 46L_47D_49P_50I_52G_53F_54S_55C_67L_ | 2.165096903 |
| 646 | 460 | TTCTGTTGTCCTGTGTGTGGGGCAGCCTTTactcagaaaggtaacctcctccgccacattaaaCTG | 46F_47C_49P_50V_52G_53A_54A_55F_67L_ | 2.163057769 |
| 647 | 1901 | TTCATCTGTCCAGTATGTGGGGCATGCTTTactcagaaaggtaacctcctccgccacattaaaCTG | 46F_47I_49P_50V_52G_53A_54C_55F_67L_ | 2.162126361 |
| 648 | 317 | TTCTGTTGTGAGATTTGTGGGGCAGTGTTTactcagaaaggtaacctcctccgccacattaaaCTG | 46F_47C_49E_50I_52G_53A_54V_55F_67L_ | 2.160770541 |
| 649 | 3910 | TTCGTGTGTGAAATTTGTGGGGCAATGTTTactcagaaaggtaacctcctccgccacattaaaCTG | 46F_47V_49E_50I_52G_53A_54M_55F_67L_ | 2.158837355 |
| 650 | 857 | TTCGAGTGTAACATCTGTGGGGCAGTATTTactcagaaaggtaacctcctccgccacattaaaCTG | 46F_47E_49N_50I_52G_53A_54V_55F_67L_ | 2.157349364 |
| 651 | 7741 | TTGATCTGTGAAATTTGCGGCTTTTGCTGCactcagaaaggtaacctcctccgccacattaaaCTG | 46L_47I_49E_50I_52G_53F_54C_55C_67L_ | 2.153961332 |
| 652 | 3667 | TTCACCTGTGAAATCTGTGGGGCAATTTTTactcagaaaggtaacctcctccgccacattaaaCTG | 46F_47T_49E_50I_52G_53A_54I_55F_67L_ | 2.151556866 |
| 653 | 1914 | TTCATATGTCCAGTCTGTGGGGCACGGTTTactcagaaaggtaacctcctccgccacattaaaCTG | 46F_47I_49P_50V_52G_53A_54R_55F_67L_ | 2.151465585 |
| 654 | 6326 | TTCCAGTGTCCAATCTGTGGGAGGCACTTTactcagaaaggtaacctcctccgccacattaaaCTG | 46F_47Q_49P_50I_52G_53R_54H_55F_67L_ | 2.151082294 |
| 655 | 3420 | TTCAGCTGTGAGATATGTGGGGCAGCGTTTactcagaaaggtaacctcctccgccacattaaaCTG | 46F_47S_49E_50I_52G_53A_54A_55F_67L_ | 2.15000089 |
| 656 | 672 | TTCGACTGTCCCATCTGTGGGGCACCGTTTactcagaaaggtaacctcctccgccacattaaaCTG | 46F_47D_49P_50I_52G_53A_54P_55F_67L_ | 2.149444333 |
| 657 | 6320 | TTTCAGTGCCCAATCTGCGGCAGGGCATTTactcagaaaggtaacctcctccgccacattaaaCTG | 46F_47Q_49P_50I_52G_53R_54A_55F_67L_ | 2.147747995 |
| 658 | 671 | TTCGATTGTCCAATCTGTGGGGCAAACTTTactcagaaaggtaacctcctccgccacattaaaCTG | 46F_47D_49P_50I_52G_53A_54N_55F_67L_ | 2.145807473 |
| 659 | 6487 | TTGTGCTGTCCTATATGCGGATTTATTTGTactcagaaaggtaacctcctccgccacattaaaCTG | 46L_47C_49P_50I_52G_53F_54I_55C_67L_ | 2.145714552 |
| 660 | 7745 | CTGATCTGTGAAATTTGTGGCTTCGGCTGTactcagaaaggtaacctcctccgccacattaaaCTG | 46L_47I_49E_50I_52G_53F_54G_55C_67L_ | 2.144620369 |
| 661 | 1786 | TTCATTTGTGAGGTTTGTGGGGCACATTTTactcagaaaggtaacctcctccgccacattaaaCTG | 46F_47I_49E_50V_52G_53A_54H_55F_67L_ | 2.139440291 |
| 662 | 1905 | TTCATTTGTCCGGTCTGTGGGGCAGGCTTTactcagaaaggtaacctcctccgccacattaaaCTG | 46F_47I_49P_50V_52G_53A_54G_55F_67L_ | 2.139042167 |
| 663 | 1799 | TTCATCTGTGAGGTGTGTGGGGCATATTTTactcagaaaggtaacctcctccgccacattaaaCTG | 46F_47I_49E_50V_52G_53A_54Y_55F_67L_ | 2.137374359 |
| 664 | 6652 | CTCCTGTGCCCAATTTGCGGATTTCCATGCactcagaaaggtaacctcctccgccacattaaaCTG | 46L_47L_49P_50I_52G_53F_54P_55C_67L_ | 2.135476975 |
| 665 | 1903 | TTCATCTGTCCTGTGTGTGGGGCAGAGTTTactcagaaaggtaacctcctccgccacattaaaCTG | 46F_47I_49P_50V_52G_53A_54E_55F_67L_ | 2.134899074 |
| 666 | 3739 | TTCACATGTAATATATGTGGGGCATACTTTactcagaaaggtaacctcctccgccacattaaaCTG | 46F_47T_49N_50I_52G_53A_54Y_55F_67L_ | 2.133642788 |
| 667 | 1750 | TTCATTTGTGAGATCTGTGGGGCAATGTTTactcagaaaggtaacctcctccgccacattaaaCTG | 46F_47I_49E_50I_52G_53A_54M_55F_67L_ | 2.13109989 |
| 668 | 3735 | TTCACTTGTAACATCTGTGGGGCATCTTTTactcagaaaggtaacctcctccgccacattaaaCTG | 46F_47T_49N_50I_52G_53A_54S_55F_67L_ | 2.127019946 |
| 669 | 661 | TTCGATTGTCCAATTTGTGGGGCATGTTTTactcagaaaggtaacctcctccgccacattaaaCTG | 46F_47D_49P_50I_52G_53A_54C_55F_67L_ | 2.126163001 |
| 670 | 1757 | TTCATCTGTGAGATTTGTGGGGCAGTGTTTactcagaaaggtaacctcctccgccacattaaaCTG | 46F_47I_49E_50I_52G_53A_54V_55F_67L_ | 2.124050604 |
| 671 | 7467 | TTCCAGTGTGAGATCTGTGGAAGAATATTTactcagaaaggtaacctcctccgccacattaaaCTG | 46F_47Q_49E_50I_52G_53R_54I_55F_67L_ | 2.122846761 |
| 672 | 1759 | TTCATTTGTGAGATCTGTGGGGCATACTTTactcagaaaggtaacctcctccgccacattaaaCTG | 46F_47I_49E_50I_52G_53A_54Y_55F_67L_ | 2.121988366 |
| 673 | 3091 | TTCCAGTGTCCCCAGTGTGGGGCAAATTTTactcagaaaggtaacctcctccgccacattaaaCTG | 46F_47Q_49P_50Q_52G_53A_54N_55F_67L_ | 2.121027429 |
| 674 | 3827 | TTCACCTGTCCCGTATGTGGGGCAATTTTTactcagaaaggtaacctcctccgccacattaaaCTG | 46F_47T_49P_50V_52G_53A_54I_55F_67L_ | 2.12099654 |
| 675 | 4393 | TTCTACTGTGAGATCTGTGGGGCACAATTTactcagaaaggtaacctcctccgccacattaaaCTG | 46F_47Y_49E_50I_52G_53A_54Q_55F_67L_ | 2.119527451 |
| 676 | 6474 | CTAGCCTGTCCAATATGCGGCTTCCGCTGTactcagaaaggtaacctcctccgccacattaaaCTG | 46L_47A_49P_50I_52G_53F_54R_55C_67L_ | 2.119240452 |
| 677 | 3592 | TTCAGCTGTCCTGTATGTGGGGCACCGTTTactcagaaaggtaacctcctccgccacattaaaCTG | 46F_47S_49P_50V_52G_53A_54P_55F_67L_ | 2.117415751 |
| 678 | 3195 | TTCAGGTGTGAAATCTGTGGGGCAAGCTTTactcagaaaggtaacctcctccgccacattaaaCTG | 46F_47R_49E_50I_52G_53A_54S_55F_67L_ | 2.117143552 |
| 679 | 3434 | TTCTCGTGTGAAATTTGTGGGGCACGTTTTactcagaaaggtaacctcctccgccacattaaaCTG | 46F_47S_49E_50I_52G_53A_54R_55F_67L_ | 2.116391852 |
| 680 | 476 | TTCTGCTGTCCCGTCTGTGGGGCAACTTTTactcagaaaggtaacctcctccgccacattaaaCTG | 46F_47C_49P_50V_52G_53A_54T_55F_67L_ | 2.114627029 |
| 681 | 2620 | TTCATGTGTCCCGTGTGTGGGGCAGCTTTTactcagaaaggtaacctcctccgccacattaaaCTG | 46F_47M_49P_50V_52G_53A_54A_55F_67L_ | 2.113531024 |
| 682 | 3666 | TTCACATGTGAGATCTGTGGGGCACACTTTactcagaaaggtaacctcctccgccacattaaaCTG | 46F_47T_49E_50I_52G_53A_54H_55F_67L_ | 2.110033048 |
| 683 | 3540 | TTCAGCTGTCCAATATGTGGGGCAGCTTTTactcagaaaggtaacctcctccgccacattaaaCTG | 46F_47S_49P_50I_52G_53A_54A_55F_67L_ | 2.109677343 |
| 684 | 421 | TTCTGTTGTCCAATTTGTGGGGCATGCTTTactcagaaaggtaacctcctccgccacattaaaCTG | 46F_47C_49P_50I_52G_53A_54C_55F_67L_ | 2.105664777 |
| 685 | 1746 | TTCATCTGTGAAATCTGTGGGGCACATTTTactcagaaaggtaacctcctccgccacattaaaCTG | 46F_47I_49E_50I_52G_53A_54H_55F_67L_ | 2.103673 |
| 686 | 840 | TTCGAGTGTAATATATGTGGGGCAGCCTTTactcagaaaggtaacctcctccgccacattaaaCTG | 46F_47E_49N_50I_52G_53A_54A_55F_67L_ | 2.101919419 |
| 687 | 6497 | CTTTGCTGCCCGATCTGCGGTTTCGTTTGCactcagaaaggtaacctcctccgccacattaaaCTG | 46L_47C_49P_50I_52G_53F_54V_55C_67L_ | 2.099430597 |
| 688 | 6579 | CTCGGGTGCCCAATCTGCGGCTTCTACTGCactcagaaaggtaacctcctccgccacattaaaCTG | 46L_47G_49P_50I_52G_53F_54Y_55C_67L_ | 2.096218941 |
| 689 | 472 | TTCTGCTGTCCTGTTTGTGGGGCACCGTTTactcagaaaggtaacctcctccgccacattaaaCTG | 46F_47C_49P_50V_52G_53A_54P_55F_67L_ | 2.095648859 |
| 690 | 4068 | TTCGTGTGTCCTGTCTGTGGGGCAAAGTTTactcagaaaggtaacctcctccgccacattaaaCTG | 46F_47V_49P_50V_52G_53A_54K_55F_67L_ | 2.094562509 |
| 691 | 428 | TTCTGTTGTCCAATTTGTGGGGCAAAATTTactcagaaaggtaacctcctccgccacattaaaCTG | 46F_47C_49P_50I_52G_53A_54K_55F_67L_ | 2.093121747 |
| 692 | 6816 | CTGTGGTGTCCCATCTGCGGCTTCACTTGTactcagaaaggtaacctcctccgccacattaaaCTG | 46L_47W_49P_50I_52G_53F_54T_55C_67L_ | 2.090418633 |
| 693 | 4313 | TTCTGGTGTCCAGTGTGTGGGGCACAGTTTactcagaaaggtaacctcctccgccacattaaaCTG | 46F_47W_49P_50V_52G_53A_54Q_55F_67L_ | 2.090079707 |
| 694 | 2274 | TTCTTGTGTGAAGTCTGTGGGGCACGGTTTactcagaaaggtaacctcctccgccacattaaaCTG | 46F_47L_49E_50V_52G_53A_54R_55F_67L_ | 2.089934187 |
| 695 | 7693 | TTATTCTGTGAGATCTGCGGTTTTCAGTGTactcagaaaggtaacctcctccgccacattaaaCTG | 46L_47F_49E_50I_52G_53F_54Q_55C_67L_ | 2.089747346 |
| 696 | 436 | TTCTGCTGTCCTATCTGTGGGGCAACCTTTactcagaaaggtaacctcctccgccacattaaaCTG | 46F_47C_49P_50I_52G_53A_54T_55F_67L_ | 2.088768126 |
| 697 | 1152 | TTCTTCTGTCCAATTTGTGGGGCACCATTTactcagaaaggtaacctcctccgccacattaaaCTG | 46F_47F_49P_50I_52G_53A_54P_55F_67L_ | 2.08768684 |
| 698 | 3892 | TTCGTGTGTGATGTATGTGGGGCACCCTTTactcagaaaggtaacctcctccgccacattaaaCTG | 46F_47V_49D_50V_52G_53A_54P_55F_67L_ | 2.086080998 |
| 699 | 6606 | CTCATCTGCCCTATATGTGGGTTTCACTGTactcagaaaggtaacctcctccgccacattaaaCTG | 46L_47I_49P_50I_52G_53F_54H_55C_67L_ | 2.083718177 |
| 700 | 6414 | TTTGTCTGCCCAATCTGCGGGAGGCGGTTCactcagaaaggtaacctcctccgccacattaaaCTG | 46F_47V_49P_50I_52G_53R_54R_55F_67L_ | 2.083292082 |
| 701 | 7846 | TTACAGTGTGAAATTTGCGGTTTTCATTGTactcagaaaggtaacctcctccgccacattaaaCTG | 46L_47Q_49E_50I_52G_53F_54H_55C_67L_ | 2.082736877 |
| 702 | 3474 | TTCTCTTGTGAGGTGTGTGGGGCAAGGTTTactcagaaaggtaacctcctccgccacattaaaCTG | 46F_47S_49E_50V_52G_53A_54R_55F_67L_ | 2.082079957 |
| 703 | 6661 | CTCATGTGCCCCATTTGCGGCTTCTGTTGTactcagaaaggtaacctcctccgccacattaaaCTG | 46L_47M_49P_50I_52G_53F_54C_55C_67L_ | 2.080379858 |
| 704 | 594 | TTCGACTGTGAGGTATGTGGGGCAAGGTTTactcagaaaggtaacctcctccgccacattaaaCTG | 46F_47D_49E_50V_52G_53A_54R_55F_67L_ | 2.079440902 |
| 705 | 6758 | CTCTCGTGCCCTATATGTGGATTTTGGTGCactcagaaaggtaacctcctccgccacattaaaCTG | 46L_47S_49P_50I_52G_53F_54W_55C_67L_ | 2.077764836 |
| 706 | 6394 | TTTACTTGCCCCATTTGTGGTCGCAGATTCactcagaaaggtaacctcctccgccacattaaaCTG | 46F_47T_49P_50I_52G_53R_54R_55F_67L_ | 2.075799915 |
| 707 | 792 | TTCGAATGTGAAATCTGTGGGGCACCCTTTactcagaaaggtaacctcctccgccacattaaaCTG | 46F_47E_49E_50I_52G_53A_54P_55F_67L_ | 2.073770086 |
| 708 | 7872 | CTCCGGTGCGAGATTTGTGGATTTCCCTGCactcagaaaggtaacctcctccgccacattaaaCTG | 46L_47R_49E_50I_52G_53F_54P_55C_67L_ | 2.073605111 |
| 709 | 4012 | TTCGTGTGTAACGTGTGTGGGGCACCATTTactcagaaaggtaacctcctccgccacattaaaCTG | 46F_47V_49N_50V_52G_53A_54P_55F_67L_ | 2.073471695 |
| 710 | 2228 | TTCCTTTGTGAAATATGTGGGGCAAAGTTTactcagaaaggtaacctcctccgccacattaaaCTG | 46F_47L_49E_50I_52G_53A_54K_55F_67L_ | 2.072555385 |
| 711 | 3235 | TTCCGATGTGAGGTGTGTGGGGCAAGCTTTactcagaaaggtaacctcctccgccacattaaaCTG | 46F_47R_49E_50V_52G_53A_54S_55F_67L_ | 2.071008081 |
| 712 | 2233 | TTCCTTTGTGAAATCTGTGGGGCACAGTTTactcagaaaggtaacctcctccgccacattaaaCTG | 46F_47L_49E_50I_52G_53A_54Q_55F_67L_ | 2.068036679 |
| 713 | 1852 | TTCATCTGTAACGTCTGTGGGGCACCTTTTactcagaaaggtaacctcctccgccacattaaaCTG | 46F_47I_49N_50V_52G_53A_54P_55F_67L_ | 2.06718292 |
| 714 | 3083 | TTCCAATGTCCTCAATGTGGGGCAGAGTTTactcagaaaggtaacctcctccgccacattaaaCTG | 46F_47Q_49P_50Q_52G_53A_54E_55F_67L_ | 2.066534427 |
| 715 | 4066 | TTCGTGTGTCCAGTGTGTGGGGCACACTTTactcagaaaggtaacctcctccgccacattaaaCTG | 46F_47V_49P_50V_52G_53A_54H_55F_67L_ | 2.065668739 |
| 716 | 7492 | TTCCGGTGTGAGATCTGCGGAAGGCCTTTTactcagaaaggtaacctcctccgccacattaaaCTG | 46F_47R_49E_50I_52G_53R_54P_55F_67L_ | 2.063508218 |
| 717 | 437 | TTCTGCTGTCCTATATGTGGGGCAGTGTTTactcagaaaggtaacctcctccgccacattaaaCTG | 46F_47C_49P_50I_52G_53A_54V_55F_67L_ | 2.062125748 |
| 718 | 2998 | TTCCAGTGTGAAGTGTGTGGGGCATGGTTTactcagaaaggtaacctcctccgccacattaaaCTG | 46F_47Q_49E_50V_52G_53A_54W_55F_67L_ | 2.061128811 |
| 719 | 6666 | CTGATGTGTCCCATCTGCGGATTTCACTGCactcagaaaggtaacctcctccgccacattaaaCTG | 46L_47M_49P_50I_52G_53F_54H_55C_67L_ | 2.060157753 |
| 720 | 853 | TTCGAGTGTAACATCTGTGGGGCACAATTTactcagaaaggtaacctcctccgccacattaaaCTG | 46F_47E_49N_50I_52G_53A_54Q_55F_67L_ | 2.058102015 |
| 721 | 461 | TTCTGCTGTCCCGTGTGTGGGGCATGTTTTactcagaaaggtaacctcctccgccacattaaaCTG | 46F_47C_49P_50V_52G_53A_54C_55F_67L_ | 2.057765317 |
| 722 | 1868 | TTCATATGTCCTATTTGTGGGGCAAAATTTactcagaaaggtaacctcctccgccacattaaaCTG | 46F_47I_49P_50I_52G_53A_54K_55F_67L_ | 2.056651536 |
| 723 | 312 | TTCTGCTGTGAGATTTGTGGGGCACCTTTTactcagaaaggtaacctcctccgccacattaaaCTG | 46F_47C_49E_50I_52G_53A_54P_55F_67L_ | 2.054282586 |
| 724 | 2515 | TTCATGTGTGAAGTGTGTGGGGCATCGTTTactcagaaaggtaacctcctccgccacattaaaCTG | 46F_47M_49E_50V_52G_53A_54S_55F_67L_ | 2.053648338 |
| 725 | 6814 | CTTTGGTGCCCAATATGTGGCTTCCGGTGCactcagaaaggtaacctcctccgccacattaaaCTG | 46L_47W_49P_50I_52G_53F_54R_55C_67L_ | 2.053515254 |
| 726 | 3824 | TTCACTTGTCCTGTGTGTGGGGCATTCTTTactcagaaaggtaacctcctccgccacattaaaCTG | 46F_47T_49P_50V_52G_53A_54F_55F_67L_ | 2.053126688 |
| 727 | 6604 | CTGATTTGTCCCATTTGCGGCTTCTTTTGCactcagaaaggtaacctcctccgccacattaaaCTG | 46L_47I_49P_50I_52G_53F_54F_55C_67L_ | 2.052180835 |
| 728 | 1796 | TTCATATGTGAAGTCTGTGGGGCAACCTTTactcagaaaggtaacctcctccgccacattaaaCTG | 46F_47I_49E_50V_52G_53A_54T_55F_67L_ | 2.050567923 |
| 729 | 7901 | CTGACGTGCGAGATATGCGGTTTCTGCTGCactcagaaaggtaacctcctccgccacattaaaCTG | 46L_47T_49E_50I_52G_53F_54C_55C_67L_ | 2.05009536 |
| 730 | 1813 | TTCATATGTAACATATGTGGGGCACAGTTTactcagaaaggtaacctcctccgccacattaaaCTG | 46F_47I_49N_50I_52G_53A_54Q_55F_67L_ | 2.049599325 |
| 731 | 3950 | TTCGTTTGTGAGGTCTGTGGGGCAATGTTTactcagaaaggtaacctcctccgccacattaaaCTG | 46F_47V_49E_50V_52G_53A_54M_55F_67L_ | 2.047506749 |
| 732 | 6804 | CTATGGTGTCCAATCTGCGGCTTTTTTTGCactcagaaaggtaacctcctccgccacattaaaCTG | 46L_47W_49P_50I_52G_53F_54F_55C_67L_ | 2.046812441 |
| 733 | 6699 | CTGAATTGCCCAATTTGTGGCTTCTATTGCactcagaaaggtaacctcctccgccacattaaaCTG | 46L_47N_49P_50I_52G_53F_54Y_55C_67L_ | 2.042152708 |
| 734 | 6553 | TTATTCTGCCCTATCTGTGGCTTTCAGTGCactcagaaaggtaacctcctccgccacattaaaCTG | 46L_47F_49P_50I_52G_53F_54Q_55C_67L_ | 2.041376334 |
| 735 | 4153 | TTCTGGTGTGAAATTTGTGGGGCACAGTTTactcagaaaggtaacctcctccgccacattaaaCTG | 46F_47W_49E_50I_52G_53A_54Q_55F_67L_ | 2.039856287 |
| 736 | 7813 | CTTATGTGCGAGATTTGTGGGTTCCAGTGCactcagaaaggtaacctcctccgccacattaaaCTG | 46L_47M_49E_50I_52G_53F_54Q_55C_67L_ | 2.038126199 |
| 737 | 6538 | TTAGAATGTCCTATCTGCGGTTTCTGGTGTactcagaaaggtaacctcctccgccacattaaaCTG | 46L_47E_49P_50I_52G_53F_54W_55C_67L_ | 2.037919548 |
| 738 | 3706 | TTCACCTGTGAGGTTTGTGGGGCACACTTTactcagaaaggtaacctcctccgccacattaaaCTG | 46F_47T_49E_50V_52G_53A_54H_55F_67L_ | 2.037678561 |
| 739 | 6127 | TTTGATTGTCCAATATGTGGCCGCATTTTTactcagaaaggtaacctcctccgccacattaaaCTG | 46F_47D_49P_50I_52G_53R_54I_55F_67L_ | 2.037224747 |
| 740 | 4193 | TTCTGGTGTGAGGTGTGTGGGGCACAGTTTactcagaaaggtaacctcctccgccacattaaaCTG | 46F_47W_49E_50V_52G_53A_54Q_55F_67L_ | 2.031957141 |
| 741 | 3711 | TTCACGTGTGAGGTGTGTGGGGCAAACTTTactcagaaaggtaacctcctccgccacattaaaCTG | 46F_47T_49E_50V_52G_53A_54N_55F_67L_ | 2.028689038 |
| 743 | 6508 | TTGGACTGTCCAATATGCGGTTTCAAATGTactcagaaaggtaacctcctccgccacattaaaCTG | 46L_47D_49P_50I_52G_53F_54K_55C_67L_ | 2.023933158 |
| 742 | 6491 | TTGTGCTGTCCAATCTGCGGGTTCAACTGCactcagaaaggtaacctcctccgccacattaaaCTG | 46L_47C_49P_50I_52G_53F_54N_55C_67L_ | 2.027027498 |
| 744 | 2635 | TTCATGTGTCCGGTCTGTGGGGCATCATTTactcagaaaggtaacctcctccgccacattaaaCTG | 46F_47M_49P_50V_52G_53A_54S_55F_67L_ | 2.021785865 |
| 745 | 3663 | TTCACCTGTGAGATATGTGGGGCAGAGTTTactcagaaaggtaacctcctccgccacattaaaCTG | 46F_47T_49E_50I_52G_53A_54E_55F_67L_ | 2.021225398 |
| 746 | 6741 | CTGAGCTGTCCCATCTGTGGCTTCTGCTGCactcagaaaggtaacctcctccgccacattaaaCTG | 46L_47S_49P_50I_52G_53F_54C_55C_67L_ | 2.015032561 |
| 747 | 4076 | TTCGTGTGTCCTGTGTGTGGGGCAACTTTTactcagaaaggtaacctcctccgccacattaaaCTG | 46F_47V_49P_50V_52G_53A_54T_55F_67L_ | 2.014349252 |
| 748 | 3555 | TTCAGTTGTCCTATCTGTGGGGCAAGCTTTactcagaaaggtaacctcctccgccacattaaaCTG | 46F_47S_49P_50I_52G_53A_54S_55F_67L_ | 2.012212385 |
| 749 | 3957 | TTCGTTTGTGAGGTCTGTGGGGCAGTGTTTactcagaaaggtaacctcctccgccacattaaaCTG | 46F_47V_49E_50V_52G_53A_54V_55F_67L_ | 2.012071443 |
| 750 | 7673 | TTAGAATGCGAAATATGTGGGTTCCAGTGTactcagaaaggtaacctcctccgccacattaaaCTG | 46L_47E_49E_50I_52G_53F_54Q_55C_67L_ | 2.006039982 |
| 751 | 3460 | TTCAGCTGTGAGGTCTGTGGGGCAGCTTTTactcagaaaggtaacctcctccgccacattaaaCTG | 46F_47S_49E_50V_52G_53A_54A_55F_67L_ | 2.00577077 |
| 752 | 6504 | TTAGATTGCCCCATTTGTGGCTTTTTTTGCactcagaaaggtaacctcctccgccacattaaaCTG | 46L_47D_49P_50I_52G_53F_54F_55C_67L_ | 2.004336353 |
| 753 | 700 | TTCGACTGTCCGGTCTGTGGGGCAGCATTTactcagaaaggtaacctcctccgccacattaaaCTG | 46F_47D_49P_50V_52G_53A_54A_55F_67L_ | 2.001611674 |
| 754 | 2475 | TTCATGTGTGAAATCTGTGGGGCATCATTTactcagaaaggtaacctcctccgccacattaaaCTG | 46F_47M_49E_50I_52G_53A_54S_55F_67L_ | 2.000402816 |
| 755 | 6695 | CTCAACTGCCCCATCTGCGGGTTTAGCTGCactcagaaaggtaacctcctccgccacattaaaCTG | 46L_47N_49P_50I_52G_53F_54S_55C_67L_ | 2.000386954 |

TABLE 2
A63 orthogonality; Estimated LogFC Coefficient calculated from the limma voom linear regression contrast of the positive vs negative read
counts for each individual mutant degron as defined relative to SEQ ID NO:2.

| | | | Annotation of amino acids according | |
|---|---|---|---|---|
| | | | to their position in SEQ ID NO: 2, | |
| | | | wherein the stated one letter amino | |
| | | | acid may be the original amino | |
| | | DNA sequence | acid found in SEQ ID NO: 2 or a | |
| | | encoding positions F146-L167 of SEQ ID NO: 2 absent any | substitution (first numerical digit | |
| SEQ ID | Degron- | substitutions defined according to the encoding sequence below and | dropped, i.e. position 146 in | |
| NO | ID | the variant amino acid sequence set out in column 4 of this Table | SEQ ID NO: 2 = 46 as stated below) | A63_logFC |
| 756 | 1512 | TTCCATTGTGAGATCTGTGGGGCACCATTTactcagaaaggtaacctcctccgccacattaaaCTG | 46F_47H_49E_50I_52G_53A_54P_55F 67L | 4.001281654 |
| 757 | 1072 | TTCTTCTGTGAGGTGTGTGGGGCACCCTTTactcagaaaggtaacctcctccgccacattaaaCTG | 46F_47F_49E_50V_52G_53A_54P_55F_67L | 3.99795163 |
| 758 | 4148 | TTCTGGTGTGAAATCTGTGGGGCAAAGTTTactcagaaaggtaacctcctccgccacattaaaCTG | 46F_47W_49E_50I_52G_53A_54K_55F_67L | 3.955125386 |
| 759 | 972 | TTCTTCTGTGACATATGTGGGGCACCCTTTactcagaaaggtaacctcctccgccacattaaaCTG | 46F_47F_49D_50I_52G_53A_54P_55F_67L | 3.919458587 |
| 760 | 108 | TTCGCTTGTGAAGTGTGTGGGGCAAAATTTactcagaaaggtaacctcctccgccacattaaaCTG | 46F_47A_49E_50V_52G_53A_54K 55F 67L | 3.917583973 |
| 761 | 1152 | TTCTTCTGTCCAATTTGTGGGGCACCATTTactcagaaaggtaacctcctccgccacattaaaCTG | 46F_47F_49P_50I_52G_53A_54P 55F 67L | 3.916446179 |
| 762 | 114 | TTCGCCTGTGAGGTGTGTGGGGCAAGATTTactcagaaaggtaacctcctccgccacattaaaCTG | 46F_47A_49E_50V_52G_53A_54R 55F 67L | 3.908374585 |
| 763 | 4388 | TTCTACTGTGAGATCTGTGGGGCAAAATTTactcagaaaggtaacctcctccgccacattaaaCTG | 46F_47Y_49E_50I_52G_53A_54K_55F_67L | 3.903486269 |
| 764 | 1381 | TTCGGCTGTCCCATTTGTGGGGCATGTTTTactcagaaaggtaacctcctccgccacattaaaCTG | 46F_47G_49P_50I_52G_53A_54C_55F_67L | 3.900507395 |
| 765 | 60 | TTCGCATGTGAGATCTGTGGGGCAGCCTTTactcagaaaggtaacctcctccgccacattaaaCTG | 46F_47A_49E_50I_52G_53A_54A 55F_67L | 3.896552562 |
| 766 | 1421 | TTCGGATGTCCCGTCTGTGGGGCATGCTTTactcagaaaggtaacctcctccgccacattaaaCTG | 46F_47G_49P_50V_52G_53A_54C_55F_67L | 3.884493345 |
| 767 | 4420 | TTCTACTGTGAAGTCTGTGGGGCAGCTTTTactcagaaaggtaacctcctccgccacattaaaCTG | 46F_47Y_49E_50V_52G_53A_54A_55F_67L | 3.872574027 |
| 768 | 115 | TTCGCTTGTGAGGTGTGTGGGGCAAGTTTTactcagaaaggtaacctcctccgccacattaaaCTG | 46F 47A 49E 50V 52G 53A 54S 55F 67L | 3.869782272 |
| 769 | 4452 | TTCTACTGTAATATTTGTGGGGCACCATTTactcagaaaggtaacctcctccgccacattaaaCTG | 46F_47Y_49N_50I_52G_53A_54P_55F_67L | 3.867603423 |
| 770 | 4332 | TTCTATTGTGATATATGTGGGGCACCGTTTactcagaaaggtaacctcctccgccacattaaaCTG | 46F_47Y_49D_50I_52G_53A_54P_55F_67L | 3.862621101 |
| 771 | 4392 | TTCTATTGTGAAATTTGTGGGGCACCATTTactcagaaaggtaacctcctccgccacattaaaCTG | 46F_47Y_49E_50I_52G_53A_54P_55F_67L | 3.860802269 |
| 772 | 1314 | TTCGGGTGTGAGGTGTGTGGGGCACGATTTactcagaaaggtaacctcctccgccacattaaaCTG | 46F 47G 49E 50V_52G 53A 54R 55F 67L | 3.858728012 |
| 773 | 1430 | TTCGGATGTCCGGTTTGTGGGGCAATGTTTactcagaaaggtaacctcctccgccacattaaaCTG | 46F_47G_49P_50V_52G_53A_54M_55F_67L | 3.858518478 |
| 774 | 4141 | TTCTGGTGTGAGATCTGTGGGGCATGCTTTactcagaaaggtaacctcctccgccacattaaaCTG | 46F_47W_49E_50I_52G_53A_54C_55F_67L | 3.852405412 |
| 775 | 74 | TTCGCTTGTGAGATCTGTGGGGCACGATTTactcagaaaggtaacctcctccgccacattaaaCTG | 46F_47A_49E_50I_52G_53A_54R_55F_67L | 3.837156224 |
| 776 | 4434 | TTCTACTGTGAGGTCTGTGGGGCAAGGTTTactcagaaaggtaacctcctccgccacattaaaCTG | 46F_47Y_49E_50V_52G_53A_54R_55F_67L | 3.835358574 |
| 777 | 4155 | TTCTGGTGTGAGATTTGTGGGGCATCTTTTactcagaaaggtaacctcctccgccacattaaaCTG | 46F_47W_49E_50I_52G_53A 54S_55F_67L | 3.826775102 |
| 778 | 1092 | TTCTTCTGTAACATCTGTGGGGCACCTTTTactcagaaaggtaacctcctccgccacattaaaCTG | 46F_47F_49N_50I_52G_53A_54P 55F 67L | 3.823843242 |
| 779 | 68 | TTCGCTTGTGAGATCTGTGGGGCAAAATTTactcagaaaggtaacctcctccgccacattaaaCTG | 46F_47A_49E_50I 52G 53A 54K 55F 67L | 3.821546085 |
| 780 | 4152 | TTCTGGTGTGAAATTTGTGGGGCACCATTTactcagaaaggtaacctcctccgccacattaaaCTG | 46F_47W_49E_50I_52G_53A_54P_55F_67L | 3.81889683 |
| 781 | 1428 | TTCGGTTGTCCGGTGTGTGGGGCAAAATTTactcagaaaggtaacctcctccgccacattaaaCTG | 46F_47G_49P_50V_52G_53A_54K_55F_67L | 3.817325045 |
| 782 | 1387 | TTCGGTTGTCCCATCTGTGGGGCAATTTTTactcagaaaggtaacctcctccgccacattaaaCTG | 46F_47G_49P_50I_52G_53A_54I_55F_67L | 3.812786225 |
| 783 | 1433 | TTCGGGTGTCCAGTTTGTGGGGCACAGTTTactcagaaaggtaacctcctccgccacattaaaCTG | 46F_47G_49P_50V_52G_53A_54Q_55F 67L | 3.796999371 |
| 784 | 1074 | TTCTTTTGTGAGGTATGTGGGGCAAGGTTTactcagaaaggtaacctcctccgccacattaaaCTG | 46F_47F_49E_50V_52G_53A_54R_55F_67L | 3.784844027 |
| 785 | 1021 | TTCTTTTGTGAAATCTGTGGGGCATGCTTTactcagaaaggtaacctcctccgccacattaaaCTG | 46F_47F_49E_50I_52G_53A_54C_55F_67L | 3.783564731 |
| 786 | 1028 | TTCTTTTGTGAGATTTGTGGGGCAAAATTTactcagaaaggtaacctcctccgccacattaaaCTG | 46F_47F_49E_50I_52G_53A_54K_55F_67L | 3.781063604 |
| 787 | 4195 | TTCTGGTGTGAGGTGTGTGGGGCATCCTTTactcagaaaggtaacctcctccgccacattaaaCTG | 46F_47W_49E_50V_52G_53A_54S_55F_67L | 3.771189109 |
| 788 | 4140 | TTCTGGTGTGAAATTTGTGGGGCAGCTTTTactcagaaaggtaacctcctccgccacattaaaCTG | 46F_47W_49E_50I_52G_53A_54A_55F_67L | 3.767017184 |
| 789 | 101 | TTCGCATGTGAGGTGTGTGGGGCATGCTTTactcagaaaggtaacctcctccgccacattaaaCTG | 46F_47A 49E_50V_52G_53A_54C_55F_67L | 3.756799906 |
| 790 | 65 | TTCGCTTGTGAAATATGTGGGGCAGGATTTactcagaaaggtaacctcctccgccacattaaaCTG | 46F_47A_49E_50I_52G_53A_54G_55F_67L | 3.75236937 |
| 791 | 1392 | TTCGGCTGTCCCATCTGTGGGGCACCTTTTactcagaaaggtaacctcctccgccacattaaaCTG | 46F_47G 49P 50I 52G 53A 54P 55F 67L | 3.743271159 |
| 792 | 4421 | TTCTACTGTGAGGTGTGTGGGGCATGCTTTactcagaaaggtaacctcctccgccacattaaaCTG | 46F_47Y_49E_50V_52G_53A_54C_55F_67L | 3.742874408 |
| 793 | 4188 | TTCTGGTGTGAAGTGTGTGGGGCAAAATTTactcagaaaggtaacctcctccgccacattaaaCTG | 46F_47W_49E_50V_52G_53A_54K_55F_67L | 3.739937553 |
| 794 | 4391 | TTCTACTGTGAAATTTGTGGGGCAAATTTTactcagaaaggtaacctcctccgccacattaaaCTG | 46F_47Y_49E_50I_52G_53A_54N_55F_67L | 3.738537755 |
| 795 | 6573 | CTGGGCTGTCCAATCTGCGGGTTTCAATGCactcagaaaggtaacctcctccgccacattaaaCTG | 46L_47G_49P_50I_52G_53F_54Q_55C_67L | 3.738347973 |
| 796 | 1427 | TTCGGATGTCCCGTCTGTGGGGCAATATTTactcagaaaggtaacctcctccgccacattaaaCTG | 46F_47G_49P_50V_52G_53A_54I 55F_67L | 3.732289446 |
| 797 | 195 | TTCGCCTGTCCTATCTGTGGGGCATCATTTactcagaaaggtaacctcctccgccacattaaaCTG | 46F_47A_49P_50I_52G_53A_54S_55F 67L | 3.730135631 |
| 798 | 6835 | TTGTACTGCCCTATATGTGGGTTCAGCTGCactcagaaaggtaacctcctccgccacattaaaCTG | 46L_47Y_49P_50I_52G_53F_54S_55C_67L | 3.727876293 |
| 799 | 180 | TTCGCATGTCCCATATGTGGGGCAGCTTTTactcagaaaggtaacctcctccgccacattaaaCTG | 46F_47A_49P_50I_52G_53A 54A 55F 67L | 3.727177051 |
| 800 | 4381 | TTCTATTGTGAGATATGTGGGGCATGCTTTactcagaaaggtaacctcctccgccacattaaaCTG | 46F_47Y_49E_50I_52G_53A_54C_55F_67L | 3.727084219 |
| 801 | 194 | TTCGCATGTCCTATTTGTGGGGCACGATTTactcagaaaggtaacctcctccgccacattaaaCTG | 46F_47A_49P_50I_52G_53A_54R_55F_67L | 3.721538265 |
| 802 | 300 | TTCTGCTGTGAAATCTGTGGGGCAGCCTTTactcagaaaggtaacctcctccgccacattaaaCTG | 46F_47C_49E_50I_52G_53A_54A_55F_67L | 3.719937882 |
| 803 | 4395 | TTCTATTGTGAGATCTGTGGGGCATCTTTTactcagaaaggtaacctcctccgccacattaaaCTG | 46F_47Y 49E 50I 52G 53A 54S 55F 67L | 3.718501114 |
| 804 | 61 | TTCGCGTGTGAAATTTGTGGGGCATGTTTTactcagaaaggtaacctcctccgccacattaaaCTG | 46F_47A 49E_50I_52G_53A 54C_55F_67L | 3.712548896 |
| 805 | 7694 | CTTTTCTGTGAAATTTGCGGTTTCAGGTGTactcagaaaggtaacctcctccgccacattaaaCTG | 46L_47F_49E_50I_52G_53F_54R_55C_67L | 3.706909269 |
| 806 | 4394 | TTCTACTGTGAAATCTGTGGGGCAAGATTTactcagaaaggtaacctcctccgccacattaaaCTG | 46F_47Y_49E_50I 52G_53A 54R 55F 67L | 3.704898671 |
| 807 | 192 | TTCGCTTGTCCCATTTGTGGGGCACCATTTactcagaaaggtaacctcctccgccacattaaaCTG | 46F_47A_49P_50I_52G_53A_54P_55F_67L | 3.700959025 |
| 808 | 4435 | TTCTACTGTGAAGTTTGTGGGGCAAGCTTTactcagaaaggtaacctcctccgccacattaaaCTG | 46F_47Y_49E_50V_52G_53A_54S_55F_67L | 3.698367166 |
| 809 | 4432 | TTCTACTGTGAAGTATGTGGGGCACCATTTactcagaaaggtaacctcctccgccacattaaaCTG | 46F_47Y_49E_50V_52G_53A_54P_55F 67L | 3.694524449 |
| 810 | 1308 | TTCGGGTGTGAAGTCTGTGGGGCAAAGTTTactcagaaaggtaacctcctccgccacattaaaCTG | 46F_47G_49E_50V_52G_53A_54K_55F_67L | 3.693231765 |
| 811 | 6475 | TTGGCGTGTCCAATTTGCGGATTCTCATGTactcagaaaggtaacctcctccgccacattaaaCTG | 46L_47A_49P_50I_52G_53F_54S_55C_67L | 3.692185679 |
| 812 | 2952 | TTCCAGTGTGAGATTTGTGGGGCACCATTTactcagaaaggtaacctcctccgccacattaaaCTG | 46F_47Q_49E_50I_52G_53A_54P_55F_67L | 3.68503127 |
| 813 | 6476 | TTAGCCTGTCCCATTTGCGGGTTTACATGCactcagaaaggtaacctcctccgccacattaaaCTG | 46L_47A_49P_50I_52G_53F_54T_55C_67L | 3.68254812 |
| 814 | 305 | TTCTGCTGTGAAATCTGTGGGGCAGGTTTTactcagaaaggtaacctcctccgccacattaaaCTG | 46F_47C_49E_50I_52G_53A_54G_55F_67L | 3.679166702 |
| 815 | 6568 | TTAGGATGCCCAATTTGCGGTTTCAAATGTactcagaaaggtaacctcctccgccacattaaaCTG | 46L_47G_49P_50I_52G_53F_54K_55C_67L | 3.673782013 |
| 816 | 1033 | TTCTTCTGTGAGATCTGTGGGGCACAGTTTactcagaaaggtaacctcctccgccacattaaaCTG | 46F_47F_49E_50I_52G_53A_54Q_55F_67L | 3.669840281 |
| 817 | 75 | TTCGCCTGTGAGATCTGTGGGGCATCATTTactcagaaaggtaacctcctccgccacattaaaCTG | 46F 47A 49E_50I 52G_53A_54S 55F 67L | 3.667602631 |
| 818 | 7955 | CTGTGGTGCGAGATCTGCGGCTTCAGCTGTactcagaaaggtaacctcctccgccacattaaaCTG | 46L_47W_49E_50I_52G_53F_54S_55C_67L | 3.664343355 |
| 819 | 1436 | TTCGGCTGTCCGGTGTGTGGGGCAACGTTTactcagaaaggtaacctcctccgccacattaaaCTG | 46F_47G_49P_50V_52G_53A_54T_55F_67L | 3.660641321 |
| 820 | 4397 | TTCTACTGTGAGATATGTGGGGCAGTATTTactcagaaaggtaacctcctccgccacattaaaCTG | 46F_47Y_49E_50I_52G_53A_54V_55F_67L | 3.660590217 |
| 821 | 7695 | CTTTTCTGCGAAATTTGTGGCTTCTCCTGCactcagaaaggtaacctcctccgccacattaaaCTG | 46L_47F_49E_50I_52G_53F_54S_55C_67L | 3.657182621 |
| 822 | 73 | TTCGCCTGTGAGATTTGTGGGGCACAATTTactcagaaaggtaacctcctccgccacattaaaCTG | 46F_47A_49E_50I_52G_53A_54Q_55F_67L | 3.656059946 |
| 823 | 1065 | TTCTTTTGTGAGGTGTGTGGGGCAGGATTTactcagaaaggtaacctcctccgccacattaaaCTG | 46F_47F_49E_50V_52G_53A_54G_55F_67L | 3.648774725 |
| 824 | 6576 | CTGGGGTGTCCAATATGCGGATTTACCTGCactcagaaaggtaacctcctccgccacattaaaCTG | 46L_47G_49P_50I_52G_53F_54T_55C_67L | 3.64785924 |
| 825 | 432 | TTCTGCTGTCCGATCTGTGGGGCACCTTTTactcagaaaggtaacctcctccgccacattaaaCTG | 46F_47C_49P_50I_52G_53A_54P_55F_67L | 3.64560421 |
| 826 | 4385 | TTCTATTGTGAGATATGTGGGGCAGGGTTTactcagaaaggtaacctcctccgccacattaaaCTG | 46F_47Y_49E_50I_52G_53A_54G_55F_67L | 3.644511835 |
| 827 | 4181 | TTCTGGTGTGAAGTCTGTGGGGCATGCTTTactcagaaaggtaacctcctccgccacattaaaCTG | 46F_47W_49E_50V_52G_53A_54C_55F_67L | 3.644077033 |
| 828 | 100 | TTCGCCTGTGAGGTCTGTGGGGCAGCCTTTactcagaaaggtaacctcctccgccacattaaaCTG | 46F_47A_49E_50V_52G_53A_54A_55F_67L | 3.643894679 |
| 829 | 7696 | CTGTTCTGTGAGATTTGTGGCTTCACGTGCactcagaaaggtaacctcctccgccacattaaaCTG | 46L_47F_49E_50I_52G_53F_54T_55C_67L | 3.6365287 |
| 830 | 1572 | TTCCATTGTAACATATGTGGGGCACCTTTTactcagaaaggtaacctcctccgccacattaaaCTG | 46F_47H_49N_50I_52G_53A_54P_55F_67L | 3.636177674 |
| 831 | 4145 | TTCTGGTGTGAAATCTGTGGGGCAGGCTTTactcagaaaggtaacctcctccgccacattaaaCTG | 46F_47W_49E_50I_52G_53A_54G_55F_67L | 3.63509147 |
| 832 | 1632 | TTCCACTGTCCAATTTGTGGGGCACCTTTTactcagaaaggtaacctcctccgccacattaaaCTG | 46F_47H_49P_50I_52G_53A_54P_55F_67L | 3.635082198 |
| 833 | 4512 | TTCTATTGTCCAATATGTGGGGCACCGTTTactcagaaaggtaacctcctccgccacattaaaCTG | 46F_47Y_49P_50I_52G_53A_54P 55F 67L | 3.632660153 |
| 834 | 7975 | CTCTACTGCGAGATTTGCGGGTTTAGCTGCactcagaaaggtaacctcctccgccacattaaaCTG | 46L_47Y_49E_50I_52G_53F_54S_55C_67L | 3.631288138 |
| 835 | 312 | TTCTGCTGTGAGATTTGTGGGGCACCTTTTactcagaaaggtaacctcctccgccacattaaaCTG | 46F_47C_49E_50I_52G_53A_54P_55F_67L | 3.626463671 |
| 836 | 4390 | TTCTACTGTGAGATATGTGGGGCAATGTTTactcagaaaggtaacctcctccgccacattaaaCTG | 46F_47Y_49E_50I_52G_53A_54M_55F_67L | 3.621714666 |
| 837 | 6575 | CTTGGCTGCCCAATTTGCGGGTTCTCTTGTactcagaaaggtaacctcctccgccacattaaaCTG | 46L 47G 49P_50I_52G_53F 54S 55C_67L | 3.617097293 |
| 838 | 3192 | TTCCGCTGTGAAATCTGTGGGGCACCCTTTactcagaaaggtaacctcctccgccacattaaaCTG | 46F_47R_49E_50I_52G_53A_54P 55F 67L | 3.616835297 |
| 839 | 185 | TTCGCCTGTCCCATATGTGGGGCAGGATTTactcagaaaggtaacctcctccgccacattaaaCTG | 46F_47A_49P_50I_52G_53A_54G_55F_67L | 3.609573794 |
| 840 | 4386 | TTCTACTGTGAGATTTGTGGGGCACACTTTactcagaaaggtaacctcctccgccacattaaaCTG | 46F_47Y_49E_50I_52G_53A_54H_55F_67L | 3.60575237 |
| 841 | 72 | TTCGCATGTGAAATATGTGGGGCACCATTTactcagaaaggtaacctcctccgccacattaaaCTG | 46F_47A_49E_50I_52G_53A_54P_55F_67L | 3.603361341 |
| 842 | 1035 | TTCTTTTGTGAGATCTGTGGGGCAAGCTTTactcagaaaggtaacctcctccgccacattaaaCTG | 46F_47F_49E_50I_52G_53A_54S_55F_67L | 3.603012898 |
| 843 | 7968 | TTATACTGCGAGATATGCGGCTTCAAATGCactcagaaaggtaacctcctccgccacattaaaCTG | 46L_47Y_49E_50I_52G_53F_54K_55C_67L | 3.600301428 |
| 844 | 1434 | TTCGGGTGTCCGGTCTGTGGGGCAAGGTTTactcagaaaggtaacctcctccgccacattaaaCTG | 46F_47G_49P_50V_52G_53A_54R 55F 67L | 3.599756455 |
| 845 | 4393 | TTCTACTGTGAGATCTGTGGGGCACAATTTactcagaaaggtaacctcctccgccacattaaaCTG | 46F_47Y_49E_50I_52G_53A_54Q_55F_67L | 3.598916726 |
| 846 | 6836 | CTCTATTGTCCCATTTGTGGGTTCACTTGCactcagaaaggtaacctcctccgccacattaaaCTG | 46L_47Y_49P_50I_52G_53F_54T_55C_67L | 3.598687721 |
| 847 | 6460 | CTAGCTTGTCCAATCTGCGGTTTTGCATGTactcagaaaggtaacctcctccgccacattaaaCTG | 46L_47A_49P_50I_52G_53F_54A 55C_67L | 3.598590468 |
| 848 | 3460 | TTCAGCTGTGAGGTCTGTGGGGCAGCTTTTactcagaaaggtaacctcctccgccacattaaaCTG | 46F_47S_49E_50V_52G_53A_54A_55F_67L | 3.596282984 |
| 849 | 220 | TTCGCCTGTCCTGTGTGTGGGGCAGCATTTactcagaaaggtaacctcctccgccacattaaaCTG | 46F_47A_49P_50V_52G_53A_54A_55F_67L | 3.595504729 |
| 850 | 2472 | TTCATGTGTGAGATTTGTGGGGCACCATTTactcagaaaggtaacctcctccgccacattaaaCTG | 46F_47M_49E_50I_52G_53A_54P_55F_67L | 3.594237807 |
| 851 | 1388 | TTCGGCTGTCCCATTTGTGGGGCAAAATTTactcagaaaggtaacctcctccgccacattaaaCTG | 46F_47G_49P_50I_52G_53A_54K 55F 67L | 3.591972639 |
| 852 | 4380 | TTCTATTGTGAAATCTGTGGGGCAGCCTTTactcagaaaggtaacctcctccgccacattaaaCTG | 46F_47Y_49E_50I_52G_53A_54A 55F_67L | 3.588653196 |
| 853 | 4261 | TTCTGGTGTCCAATCTGTGGGGCATGCTTTactcagaaaggtaacctcctccgccacattaaaCTG | 46F_47W_49P_50I_52G_53A_54C_55F_67L | 3.58451213 |
| 854 | 4437 | TTCTACTGTGAGGTGTGTGGGGCAGTGTTTactcagaaaggtaacctcctccgccacattaaaCTG | 46F_47Y_49E_50V_52G_53A_54V_55F_67L | 3.584152066 |
| 855 | 974 | TTCTTTTGTGACATTTGTGGGGCAAGATTTactcagaaaggtaacctcctccgccacattaaaCTG | 46F_47F_49D_50I_52G_53A_54R_55F_67L | 3.583703667 |
| 856 | 1393 | TTCGGCTGTCCAATTTGTGGGGCACAGTTTactcagaaaggtaacctcctccgccacattaaaCTG | 46F_47G_49P_50I_52G_53A_54Q_55F_67L | 3.581097712 |
| 857 | 1301 | TTCGGGTGTGAAGTTTGTGGGGCATGTTTTactcagaaaggtaacctcctccgccacattaaaCTG | 46F_47G_49E_50V_52G_53A_54C_55F_67L | 3.580434895 |
| 858 | 232 | TTCGCTTGTCCTGTGTGTGGGGCACCCTTTactcagaaaggtaacctcctccgccacattaaaCTG | 46F_47A_49P_50V_52G_53A_54P_55F_67L | 3.578001162 |
| 859 | 4396 | TTCTACTGTGAGATCTGTGGGGCAACATTTactcagaaaggtaacctcctccgccacattaaaCTG | 46F_47Y_49E_50I_52G_53A_54T_55F_67L | 3.575199458 |
| 860 | 3475 | TTCAGCTGTGAAGTGTGTGGGGCAAGTTTTactcagaaaggtaacctcctccgccacattaaaCTG | 46F_47S_49E_50V_52G_53A_54S_55F_67L | 3.575174393 |
| 861 | 7976 | CTCTATTGCGAAATATGCGGTTTCACATGCactcagaaaggtaacctcctccgccacattaaaCTG | 46L_47Y_49E_50I_52G_53F_54T_55C_67L | 3.569790529 |
| 862 | 340 | TTCTGTTGTGAAGTGTGTGGGGCAGCCTTTactcagaaaggtaacctcctccgccacattaaaCTG | 46F_47C_49E_50V_52G_53A_54A_55F_67L | 3.569732008 |
| 863 | 1380 | TTCGGGTGTCCCATATGTGGGGCAGCTTTTactcagaaaggtaacctcctccgccacattaaaCTG | 46F_47G_49P_50I_52G_53A_54A_55F_67L | 3.567204659 |
| 864 | 3435 | TTCTCGTGTGAGATCTGTGGGGCATCGTTTactcagaaaggtaacctcctccgccacattaaaCTG | 46F_47S_49E_50I_52G_53A_54S_55F_67L | 3.564099343 |
| 865 | 6479 | CTTGCATGCCCGATCTGCGGCTTTTACTGTactcagaaaggtaacctcctccgccacattaaaCTG | 46L_47A_49P_50I_52G_53F_54Y_55C_67L | 3.562293937 |
| 866 | 3420 | TTCAGCTGTGAGATATGTGGGGCAGCGTTTactcagaaaggtaacctcctccgccacattaaaCTG | 46F_47S_49E_50I_52G_53A_54A_55F_67L | 3.561520837 |
| 867 | 6821 | TTGTATTGTCCCATTTGTGGCTTTTGTTGTactcagaaaggtaacctcctccgccacattaaaCTG | 46L 47Y 49P 50I_52G_53F 54C_55C_67L | 3.561133401 |
| 868 | 1397 | TTCGGGTGTCCTATCTGTGGGGCAGTGTTTactcagaaaggtaacctcctccgccacattaaaCTG | 46F_47G_49P_50I_52G_53A_54V_55F_67L | 3.557063345 |
| 869 | 1437 | TTCGGCTGTCCGGTGTGTGGGGCAGTGTTTactcagaaaggtaacctcctccgccacattaaaCTG | 46F_47G_49P_50V_52G_53A_54V_55F_67L | 3.555085048 |
| 870 | 7956 | CTCTGGTGTGAAATTTGCGGATTCACCTGCactcagaaaggtaacctcctccgccacattaaaCTG | 46L_47W_49E_50I_52G_53F_54T_55C_67L | 3.552917006 |
| 871 | 7974 | CTGTATTGTGAAATTTGCGGCTTTCGCTGCactcagaaaggtaacctcctccgccacattaaaCTG | 46L 47Y 49E 50I_52G 53F 54R 55C 67L | 3.552716402 |
| 872 | 1061 | TTCTTCTGTGAGGTGTGTGGGGCATGCTTTactcagaaaggtaacctcctccgccacattaaaCTG | 46F_47F_49E_50V_52G_53A_54C_55F_67L | 3.552559498 |
| 873 | 6555 | TTGTTTTGTCCTATATGCGGGTTTTCATGCactcagaaaggtaacctcctccgccacattaaaCTG | 46L_47F_49P_50I_52G_53F_54S_55C_67L | 3.55174814 |
| 874 | 1020 | TTCTTCTGTGAGATCTGTGGGGCAGCTTTTactcagaaaggtaacctcctccgccacattaaaCTG | 46F_47F_49E_50I_52G_53A_54A_55F_67L | 3.549271521 |
| 875 | 4383 | TTCTACTGTGAAATCTGTGGGGCAGAGTTTactcagaaaggtaacctcctccgccacattaaaCTG | 46F_47Y_49E_50I_52G_53A_54E_55F_67L | 3.54650753 |
| 876 | 235 | TTCGCCTGTCCCGTGTGTGGGGCAAGCTTTactcagaaaggtaacctcctccgccacattaaaCTG | 46F_47A_49P_50V_52G_53A_54S_55F_67L | 3.544791962 |
| 877 | 1552 | TTCCACTGTGAGGTGTGTGGGGCACCTTTTactcagaaaggtaacctcctccgccacattaaaCTG | 46F_47H_49E_50V_52G_53A_54P_55F_67L | 3.540407044 |
| 878 | 4153 | TTCTGGTGTGAAATTTGTGGGGCACAGTTTactcagaaaggtaacctcctccgccacattaaaCTG | 46F_47W_49E_50I_52G_53A_54Q_55F_67L | 3.539997925 |
| 879 | 3432 | TTCTCCTGTGAGATTTGTGGGGCACCCTTTactcagaaaggtaacctcctccgccacattaaaCTG | 46F_47S_49E_50I_52G_53A_54P_55F_67L | 3.536393848 |
| 880 | 6579 | CTCGGGTGCCCAATCTGCGGCTTCTACTGCactcagaaaggtaacctcctccgccacattaaaCTG | 46L_47G_49P_50I_52G_53F_54Y_55C_67L | 3.534951607 |
| 881 | 1192 | TTCTTTTGTCCAGTGTGTGGGGCACCTTTTactcagaaaggtaacctcctccgccacattaaaCTG | 46F_47F_49P_50V_52G_53A_54P_55F_67L | 3.533329612 |
| 882 | 1068 | TTCTTTTGTGAGGTGTGTGGGGCAAAATTTactcagaaaggtaacctcctccgccacattaaaCTG | 46F_47F_49E_50V_52G_53A_54K_55F 67L | 3.532055405 |
| 883 | 3474 | TTCTCTTGTGAGGTGTGTGGGGCAAGGTTTactcagaaaggtaacctcctccgccacattaaaCTG | 46F_47S_49E_50V_52G_53A_54R_55F 67L | 3.531562235 |
| 884 | 1034 | TTCTTTTGTGAAATCTGTGGGGCAAGGTTTactcagaaaggtaacctcctccgccacattaaaCTG | 46F_47F_49E_50I_52G_53A_54R_55F_67L | 3.528565968 |
| 885 | 7973 | CTCTATTGCGAGATCTGTGGATTTCAGTGTactcagaaaggtaacctcctccgccacattaaaCTG | 46L_47Y_49E_50I_52G_53F_54Q_55C_67L | 3.527732848 |
| 886 | 4433 | TTCTATTGTGAGGTTTGTGGGGCACAATTTactcagaaaggtaacctcctccgccacattaaaCTG | 46F_47Y_49E_50V_52G_53A_54Q_55F_67L | 3.527353789 |
| 887 | 67 | TTCGCTTGTGAAATTTGTGGGGCAATATTTactcagaaaggtaacctcctccgccacattaaaCTG | 46F_47A_49E_50I_52G_53A_54I_55F_67L | 3.51525271 |
| 888 | 1025 | TTCTTCTGTGAAATTTGTGGGGCAGGGTTTactcagaaaggtaacctcctccgccacattaaaCTG | 46F_47F_49E_50I_52G_53A_54G_55F_67L | 3.513143003 |
| 889 | 1505 | TTCCATTGTGAAATCTGTGGGGCAGGCTTTactcagaaaggtaacctcctccgccacattaaaCTG | 46F_47H_49E_50I_52G_53A_54G_55F_67L | 3.507302566 |
| 890 | 7614 | CTCGCTTGTGAAATTTGTGGTTTCAGATGTactcagaaaggtaacctcctccgccacattaaaCTG | 46L_47A_49E_50I_52G_53F_54R_55C_67L | 3.501505454 |
| 891 | 1014 | TTCTTTTGTGACGTGTGTGGGGCACGTTTTactcagaaaggtaacctcctccgccacattaaaCTG | 46F_47F_49D_50V_52G_53A_54R_55F_67L | 3.498007962 |
| 892 | 4501 | TTCTATTGTCCTATCTGTGGGGCATGCTTTactcagaaaggtaacctcctccgccacattaaaCTG | 46F_47Y_49P_50I_52G_53A_54C_55F_67L | 3.497542755 |
| 893 | 1268 | TTCGGCTGTGAGATCTGTGGGGCAAAGTTTactcagaaaggtaacctcctccgccacattaaaCTG | 46F_47G_49E_50I_52G_53A_54K 55F 67L | 3.493028339 |
| 894 | 234 | TTCGCCTGTCCTGTCTGTGGGGCACGGTTTactcagaaaggtaacctcctccgccacattaaaCTG | 46F_47A 49P_50V_52G_53A 54R 55F_67L | 3.487427584 |
| 895 | 113 | TTCGCATGTGAGGTGTGTGGGGCACAGTTTactcagaaaggtaacctcctccgccacattaaaCTG | 46F_47A_49E_50V_52G_53A_54Q_55F_67L | 3.485208077 |
| 896 | 4428 | TTCTATTGTGAAGTATGTGGGGCAAAGTTTactcagaaaggtaacctcctccgccacattaaaCTG | 46F_47Y_49E_50V_52G_53A_54K_55F_67L | 3.483845744 |
| 897 | 2832 | TTCAACTGTCCTATTTGTGGGGCACCTTTTactcagaaaggtaacctcctccgccacattaaaCTG | 46F_47N_49P_50I_52G_53A_54P_55F_67L | 3.482733351 |
| 898 | 1273 | TTCGGCTGTGAGATATGTGGGGCACAGTTTactcagaaaggtaacctcctccgccacattaaaCTG | 46F_47G_49E_50I_52G_53A_54Q_55F_67L | 3.481695038 |
| 899 | 7616 | TTAGCCTGCGAGATCTGCGGGTTTACCTGTactcagaaaggtaacctcctccgccacattaaaCTG | 46L_47A_49E_50I_52G_53F_54T_55C_67L | 3.481554964 |
| 900 | 181 | TTCGCCTGTCCTATTTGTGGGGCATGCTTTactcagaaaggtaacctcctccgccacattaaaCTG | 46F_47A_49P_50I_52G_53A_54C_55F_67L | 3.476793812 |
| 901 | 4552 | TTCTATTGTCCAGTGTGTGGGGCACCTTTTactcagaaaggtaacctcctccgccacattaaaCTG | 46F_47Y 49P_50V_52G_53A 54P 55F 67L | 3.474980067 |
| 902 | 1060 | TTCTTCTGTGAGGTTTGTGGGGCAGCTTTTactcagaaaggtaacctcctccgccacattaaaCTG | 46F_47F_49E_50V_52G_53A_54A_55F 67L | 3.470353959 |
| 903 | 6187 | TTCGGCTGCCCTATTTGCGGCCGCATTTTCactcagaaaggtaacctcctccgccacattaaaCTG | 46F_47G_49P_50I_52G_53R_54I_55F_67L | 3.465466284 |
| 904 | 12 | TTCGCTTGTGATATCTGTGGGGCACCTTTTactcagaaaggtaacctcctccgccacattaaaCTG | 46F_47A_49D_50I_52G_53A_54P_55F_67L | 3.464169578 |
| 905 | 2705 | TTCAATTGTGAGATCTGTGGGGCAGGCTTTactcagaaaggtaacctcctccgccacattaaaCTG | 46F_47N_49E_50I 52G_53A_54G 55F_67L | 3.462921012 |
| 906 | 1396 | TTCGGATGTCCCATCTGTGGGGCAACATTTactcagaaaggtaacctcctccgccacattaaaCTG | 46F_47G_49P_50I_52G_53A_54T_55F 67L | 3.460436842 |
| 907 | 14 | TTCGCGTGTGACATTTGTGGGGCACGATTTactcagaaaggtaacctcctccgccacattaaaCTG | 46F_47A_49D_50I_52G_53A_54R_55F 67L | 3.460059382 |
| 908 | 1037 | TTCTTTTGTGAGATCTGTGGGGCAGTGTTTactcagaaaggtaacctcctccgccacattaaaCTG | 46F_47F_49E_50I_52G_53A_54V_55F_67L | 3.45940014 |
| 909 | 4146 | TTCTGGTGTGAGATCTGTGGGGCACACTTTactcagaaaggtaacctcctccgccacattaaaCTG | 46F_47W_49E_50I_52G_53A_54H_55F_67L | 3.453877418 |
| 910 | 3232 | TTCAGGTGTGAGGTTTGTGGGGCACCGTTTactcagaaaggtaacctcctccgccacattaaaCTG | 46F_47R_49E_50V_52G_53A_54P_55F_67L | 3.453467792 |
| 911 | 355 | TTCTGTTGTGAGGTCTGTGGGGCAAGTTTTactcagaaaggtaacctcctccgccacattaaaCTG | 46F_47C_49E_50V_52G_53A_54S_55F_67L | 3.452567941 |
| 912 | 6833 | CTCTACTGCCCTATTTGCGGGTTCCAGTGCactcagaaaggtaacctcctccgccacattaaaCTG | 46L_47Y_49P_50I_52G_53F_54Q_55C_67L | 3.450099249 |
| 913 | 1075 | TTCTTTTGTGAAGTCTGTGGGGCAAGCTTTactcagaaaggtaacctcctccgccacattaaaCTG | 46F_47F_49E_50V_52G_53A_54S_55F_67L | 3.448257514 |
| 914 | 4193 | TTCTGGTGTGAGGTGTGTGGGGCACAGTTTactcagaaaggtaacctcctccgccacattaaaCTG | 46F_47W_49E_50V_52G_53A_54Q_55F_67L | 3.443777078 |
| 915 | 6473 | CTTGCCTGCCCAATTTGTGGGTTTCAGTGCactcagaaaggtaacctcctccgccacattaaaCTG | 46L_47A_49P_50I_52G_53F_54Q_55C_67L | 3.440114783 |
| 916 | 3312 | TTCCGCTGTCCCATCTGTGGGGCACCATTTactcagaaaggtaacctcctccgccacattaaaCTG | 46F_47R_49P_50I_52G_53A_54P_55F_67L | 3.43983926 |
| 917 | 6541 | CTGTTCTGCCCCATATGTGGGTTCTGTTGTactcagaaaggtaacctcctccgccacattaaaCTG | 46L_47F_49P_50I_52G_53F_54C_55C_67L | 3.437469618 |
| 918 | 2715 | TTCAATTGTGAGATATGTGGGGCAAGTTTTactcagaaaggtaacctcctccgccacattaaaCTG | 46F_47N_49E_50I_52G_53A_54S_55F_67L | 3.43230991 |
| 919 | 7693 | TTATTCTGTGAGATCTGCGGTTTTCAGTGTactcagaaaggtaacctcctccgccacattaaaCTG | 46L_47F_49E_50I_52G_53F_54Q_55C_67L | 3.428319354 |
| 920 | 188 | TTCGCGTGTCCCATCTGTGGGGCAAAGTTTactcagaaaggtaacctcctccgccacattaaaCTG | 46F_47A_49P_50I_52G_53A_54K_55F_67L | 3.427819553 |
| 921 | 4387 | TTCTACTGTGAAATTTGTGGGGCAATCTTTactcagaaaggtaacctcctccgccacattaaaCTG | 46F_47Y_49E_50I_52G_53A_54I_55F_67L | 3.426042714 |
| 922 | 4154 | TTCTGGTGTGAAATTTGTGGGGCACGTTTTactcagaaaggtaacctcctccgccacattaaaCTG | 46F_47W_49E_50I_52G_53A_54R_55F_67L | 3.425617702 |
| 923 | 225 | TTCGCTTGTCCCGTTTGTGGGGCAGGGTTTactcagaaaggtaacctcctccgccacattaaaCTG | 46F_47A_49P_50V_52G_53A_54G_55F_67L | 3.422407965 |
| 924 | 6570 | CTGGGCTGTCCTATCTGTGGATTTATGTGCactcagaaaggtaacctcctccgccacattaaaCTG | 46L_47G_49P_50I_52G_53F_54M_55C_67L | 3.421998875 |
| 925 | 0 | TTCGCCTGTGATATCTGTGGGGCAGCTTTTactcagaaaggtaacctcctccgccacattaaaCTG | 46F_47A_49D_50I_52G_53A_54A_55F_67L | 3.420240838 |
| 926 | 2872 | TTCAATTGTCCAGTGTGTGGGGCACCCTTTactcagaaaggtaacctcctccgccacattaaaCTG | 46F_47N_49P_50V_52G_53A_54P_55F_67L | 3.413999722 |
| 927 | 4151 | TTCTGGTGTGAGATATGTGGGGCAAATTTTactcagaaaggtaacctcctccgccacattaaaCTG | 46F_47W_49E_50I_52G_53A_54N_55F_67L | 3.412947533 |
| 928 | 973 | TTCTTTTGTGACATTTGTGGGGCACAATTTactcagaaaggtaacctcctccgccacattaaaCTG | 46F_47F_49D_50I_52G_53A_54Q_55F 67L | 3.410045703 |
| 929 | 7615 | CTGGCGTGTGAAATTTGTGGATTCAGTTGCactcagaaaggtaacctcctccgccacattaaaCTG | 46L 47A_49E_50I_52G_53F_54S_55C_67L | 3.409775787 |
| 930 | 1032 | TTCTTTTGTGAAATCTGTGGGGCACCCTTTactcagaaaggtaacctcctccgccacattaaaCTG | 46F_47F_49E_50I_52G_53A_54P_55F_67L | 3.403825188 |
| 931 | 105 | TTCGCCTGTGAAGTATGTGGGGCAGGCTTTactcagaaaggtaacctcctccgccacattaaaCTG | 46F_47A_49E_50V_52G_53A_54G_55F_67L | 3.403758587 |
| 932 | 1261 | TTCGGCTGTGAAATCTGTGGGGCATGCTTTactcagaaaggtaacctcctccgccacattaaaCTG | 46F_47G_49E_50I_52G_53A_54C_55F_67L | 3.403355664 |
| 933 | 4301 | TTCTGGTGTCCGGTTTGTGGGGCATGCTTTactcagaaaggtaacctcctccgccacattaaaCTG | 46F_47W_49P_50V_52G_53A_54C_55F_67L | 3.403123771 |
| 934 | 4423 | TTCTATTGTGAAGTTTGTGGGGCAGAGTTTactcagaaaggtaacctcctccgccacattaaaCTG | 46F_47Y_49E_50V_52G_53A_54E_55F_67L | 3.402678689 |
| 935 | 1027 | TTCTTCTGTGAGATCTGTGGGGCAATTTTTactcagaaaggtaacctcctccgccacattaaaCTG | 46F 47F 49E 50I 52G 53A 54I 55F_67L | 3.402156681 |
| 936 | 7608 | CTGGCGTGTGAAATCTGTGGCTTTAAGTGCactcagaaaggtaacctcctccgccacattaaaCTG | 46L_47A_49E_50I_52G_53F_54K_55C_67L | 3.401801753 |
| 937 | 228 | TTCGCATGTCCTGTGTGTGGGGCAAAATTTactcagaaaggtaacctcctccgccacattaaaCTG | 46F_47A_49P_50V_52G_53A_54K_55F_67L | 3.397582313 |
| 938 | 1394 | TTCGGGTGTCCAATTTGTGGGGCACGCTTTactcagaaaggtaacctcctccgccacattaaaCTG | 46F_47G_49P_50I_52G_53A_54R_55F_67L | 3.39490249 |
| 939 | 6556 | CTGTTTTGTCCCATCTGCGGGTTCACATGTactcagaaaggtaacctcctccgccacattaaaCTG | 46L 47F_49P_50I_52G_53F_54T_55C_67L | 3.394538209 |
| 940 | 2712 | TTCAATTGTGAAATATGTGGGGCACCTTTTactcagaaaggtaacctcctccgccacattaaaCTG | 46F 47N 49E 50I 52G 53A 54P 55F 67L | 3.39442123 |
| 941 | 7954 | CTTTGGTGTGAAATCTGCGGATTCAGATGTactcagaaaggtaacctcctccgccacattaaaCTG | 46L_47W_49E_50I_52G_53F_54R_55C_67L | 3.39370091 |
| 942 | 196 | TTCGCCTGTCCAATATGTGGGGCAACTTTTactcagaaaggtaacctcctccgccacattaaaCTG | 46F_47A_49P_50I_52G_53A_54T_55F_67L | 3.392861292 |
| 943 | 1435 | TTCGGCTGTCCGGTGTGTGGGGCATCATTTactcagaaaggtaacctcctccgccacattaaaCTG | 46F_47G_49P_50V_52G_53A_54S_55F_67L | 3.389638299 |
| 944 | 3425 | TTCTCTTGTGAGATCTGTGGGGCAGGATTTactcagaaaggtaacctcctccgccacattaaaCTG | 46F_47S_49E_50I_52G_53A 54G 55F 67L | 3.389113685 |
| 945 | 221 | TTCGCATGTCCAGTATGTGGGGCATGTTTTactcagaaaggtaacctcctccgccacattaaaCTG | 46F_47A_49P_50V_52G_53A_54C_55F_67L | 3.387922266 |
| 946 | 4180 | TTCTGGTGTGAGGTCTGTGGGGCAGCCTTTactcagaaaggtaacctcctccgccacattaaaCTG | 46F_47W_49E_50V_52G_53A 54A 55F 67L | 3.38716432 |
| 947 | 7970 | CTCTATTGTGAGATCTGTGGATTTATGTGTactcagaaaggtaacctcctccgccacattaaaCTG | 46L_47Y_49E_50I_52G_53F_54M_55C_67L | 3.383188633 |
| 948 | 3072 | TTCCAATGTCCTATTTGTGGGGCACCATTTactcagaaaggtaacctcctccgccacattaaaCTG | 46F_47Q_49P_50I_52G_53A_54P_55F 67L | 3.383050059 |
| 949 | 4384 | TTCTATTGTGAGATCTGTGGGGCATTTTTTactcagaaaggtaacctcctccgccacattaaaCTG | 46F_47Y_49E_50I_52G_53A 54F 55F 67L | 3.382805634 |
| 950 | 6474 | CTAGCCTGTCCAATATGCGGCTTCCGCTGTactcagaaaggtaacctcctccgccacattaaaCTG | 46L_47A_49P_50I_52G_53F_54R_55C_67L | 3.373725893 |
| 951 | 4320 | TTCTACTGTGACATATGTGGGGCAGCCTTTactcagaaaggtaacctcctccgccacattaaaCTG | 46F_47Y_49D_50I_52G_53A_54A_55F_67L | 3.373331194 |
| 952 | 1315 | TTCGGCTGTGAAGTTTGTGGGGCAAGTTTTactcagaaaggtaacctcctccgccacattaaaCTG | 46F_47G_49E_50V_52G_53A_54S_55F_67L | 3.37094192 |
| 953 | 7681 | CTGTTTTGTGAAATTTGTGGATTTTGTTGTactcagaaaggtaacctcctccgccacattaaaCTG | 46L_47F_49E_50I_52G_53F_54C_55C_67L | 3.370799497 |
| 954 | 6553 | TTATTCTGCCCTATCTGTGGCTTTCAGTGCactcagaaaggtaacctcctccgccacattaaaCTG | 46L_47F_49P_50I_52G_53F_54Q_55C_67L | 3.361538495 |
| 955 | 4194 | TTCTGGTGTGAGGTTTGTGGGGCACGCTTTactcagaaaggtaacctcctccgccacattaaaCTG | 46F_47W_49E_50V_52G_53A_54R_55F_67L | 3.360804438 |
| 956 | 1073 | TTCTTCTGTGAGGTCTGTGGGGCACAGTTTactcagaaaggtaacctcctccgccacattaaaCTG | 46F_47F_49E_50V_52G_53A_54Q_55F_67L | 3.360259853 |
| 957 | 2592 | TTCATGTGTCCCATATGTGGGGCACCCTTTactcagaaaggtaacctcctccgccacattaaaCTG | 46F_47M_49P_50I_52G_53A_54P_55F_67L | 3.358281956 |
| 958 | 308 | TTCTGCTGTGAGATCTGTGGGGCAAAGTTTactcagaaaggtaacctcctccgccacattaaaCTG | 46F_47C_49E_50I_52G_53A_54K_55F_67L | 3.354205068 |
| 959 | 674 | TTCGACTGTCCTATTTGTGGGGCAAGATTTactcagaaaggtaacctcctccgccacattaaaCTG | 46F_47D_49P_50I_52G_53A_54R_55F_67L | 3.353917127 |
| 960 | 3555 | TTCAGTTGTCCTATCTGTGGGGCAAGCTTTactcagaaaggtaacctcctccgccacattaaaCTG | 46F_47S_49P_50I_52G_53A_54S_55F_67L | 3.353234273 |
| 961 | 315 | TTCTGCTGTGAGATCTGTGGGGCATCATTTactcagaaaggtaacctcctccgccacattaaaCTG | 46F_47C_49E_50I_52G_53A_54S_55F_67L | 3.352419797 |
| 962 | 1194 | TTCTTCTGTCCTGTGTGTGGGGCACGATTTactcagaaaggtaacctcctccgccacattaaaCTG | 46F_47F_49P_50V_52G_53A_54R_55F_67L | 3.351238643 |
| 963 | 7961 | TTGTACTGTGAGATCTGCGGATTCTGTTGTactcagaaaggtaacctcctccgccacattaaaCTG | 46L_47Y_49E_50I_52G_53F_54C_55C_67L | 3.348129258 |
| 964 | 968 | TTCTTTTGTGATATCTGTGGGGCAAAGTTTactcagaaaggtaacctcctccgccacattaaaCTG | 46F_47F_49D_50I_52G_53A_54K_55F_67L | 3.347359014 |
| 965 | 345 | TTCTGTTGTGAGGTCTGTGGGGCAGGCTTTactcagaaaggtaacctcctccgccacattaaaCTG | 46F_47C_49E_50V_52G_53A_54G_55F_67L | 3.345112007 |
| 966 | 1030 | TTCTTTTGTGAGATCTGTGGGGCAATGTTTactcagaaaggtaacctcctccgccacattaaaCTG | 46F_47F_49E_50I_52G_53A_54M_55F_67L | 3.343780945 |
| 967 | 1026 | TTCTTTTGTGAGATCTGTGGGGCACACTTTactcagaaaggtaacctcctccgccacattaaaCTG | 46F_47F_49E_50I_52G_53A_54H_55F_67L | 3.342848637 |
| 968 | 1432 | TTCGGGTGTCCAGTGTGTGGGGCACCCTTTactcagaaaggtaacctcctccgccacattaaaCTG | 46F_47G_49P_50V_52G_53A_54P_55F_67L | 3.341372212 |
| 969 | 420 | TTCTGCTGTCCTATCTGTGGGGCAGCCTTTactcagaaaggtaacctcctccgccacattaaaCTG | 46F_47C_49P 50I 52G_53A 54A 55F 67L | 3.335947618 |
| 970 | 76 | TTCGCCTGTGAAATCTGTGGGGCAACATTTactcagaaaggtaacctcctccgccacattaaaCTG | 46F_47A_49E_50I_52G_53A 54T 55F 67L | 3.33441927 |
| 971 | 4157 | TTCTGGTGTGAAATTTGTGGGGCAGTATTTactcagaaaggtaacctcctccgccacattaaaCTG | 46F_47W_49E_50I_52G_53A_54V_55F_67L | 3.334077253 |
| 972 | 4150 | TTCTGGTGTGAGATATGTGGGGCAATGTTTactcagaaaggtaacctcctccgccacattaaaCTG | 46F_47W_49E_50I_52G_53A_54M_55F_67L | 3.333402382 |
| 973 | 3595 | TTCAGCTGTCCCGTTTGTGGGGCATCCTTTactcagaaaggtaacctcctccgccacattaaaCTG | 46F_47S 49P 50V 52G_53A 54S 55F 67L | 3.331072843 |
| 974 | 6595 | TTACATTGCCCAATATGTGGCTTCTCCTGTactcagaaaggtaacctcctccgccacattaaaCTG | 46L_47H_49P_50I_52G_53F_54S_55C_67L | 3.330983574 |
| 975 | 4191 | TTCTGGTGTGAGGTTTGTGGGGCAAACTTTactcagaaaggtaacctcctccgccacattaaaCTG | 46F_47W 49E_50V_52G_53A_54N 55F 67L | 3.330448773 |
| 976 | 1390 | TTCGGCTGTCCCATTTGTGGGGCAATGTTTactcagaaaggtaacctcctccgccacattaaaCTG | 46F_47G_49P_50I_52G_53A 54M 55F 67L | 3.327060331 |
| 977 | 193 | TTCGCCTGTCCCATCTGTGGGGCACAATTTactcagaaaggtaacctcctccgccacattaaaCTG | 46F_47A_49P_50I_52G_53A_54Q_55F_67L | 3.326232218 |
| 978 | 4321 | TTCTACTGTGACATTTGTGGGGCATGTTTTactcagaaaggtaacctcctccgccacattaaaCTG | 46F_47Y_49D_50I_52G_53A 54C 55F 67L | 3.325024596 |
| 979 | 116 | TTCGCATGTGAGGTTTGTGGGGCAACCTTTactcagaaaggtaacctcctccgccacattaaaCTG | 46F_47A_49E_50V_52G_53A_54T_55F_67L | 3.323653308 |
| 980 | 1420 | TTCGGTTGTCCGGTCTGTGGGGCAGCCTTTactcagaaaggtaacctcctccgccacattaaaCTG | 46F_47G_49P_50V_52G_53A_54A_55F_67L | 3.319573002 |
| 981 | 1310 | TTCGGATGTGAGGTTTGTGGGGCAATGTTTactcagaaaggtaacctcctccgccacattaaaCTG | 46F_47G_49E_50V_52G_53A_54M_55F_67L | 3.319359362 |
| 982 | 4430 | TTCTATTGTGAAGTTTGTGGGGCAATGTTTactcagaaaggtaacctcctccgccacattaaaCTG | 46F_47Y_49E_50V_52G_53A_54M_55F_67L | 3.316577423 |
| 983 | 66 | TTCGCCTGTGAAATTTGTGGGGCACACTTTactcagaaaggtaacctcctccgccacattaaaCTG | 46F_47A 49E_50I 52G_53A 54H 55F_67L | 3.316569039 |
| 984 | 435 | TTCTGTTGTCCAATTTGTGGGGCATCCTTTactcagaaaggtaacctcctccgccacattaaaCTG | 46F_47C_49P_50I_52G_53A_54S_55F_67L | 3.315735382 |
| 985 | 6464 | TTGGCGTGTCCCATCTGTGGCTTTTTTTGCactcagaaaggtaacctcctccgccacattaaaCTG | 46L_47A_49P_50I_52G_53F_54F_55C_67L | 3.314831443 |
| 986 | 4081 | TTCTGGTGTGATATCTGTGGGGCATGCTTTactcagaaaggtaacctcctccgccacattaaaCTG | 46F_47W_49D_50I_52G_53A_54C_55F_67L | 3.314229452 |
| 987 | 6825 | TTGTACTGTCCCATCTGTGGTTTCGGTTGCactcagaaaggtaacctcctccgccacattaaaCTG | 46L 47Y_49P_50I_52G_53F_54G_55C_67L | 3.313964583 |
| 988 | 4425 | TTCTACTGTGAGGTGTGTGGGGCAGGATTTactcagaaaggtaacctcctccgccacattaaaCTG | 46F_47Y_49E_50V_52G_53A_54G_55F_67L | 3.312629555 |
| 989 | 4334 | TTCTATTGTGATATCTGTGGGGCAAGATTTactcagaaaggtaacctcctccgccacattaaaCTG | 46F_47Y_49D_50I_52G_53A_54R_55F_67L | 3.312181783 |
| 990 | 6815 | CTGTGGTGCCCGATCTGTGGATTTAGCTGCactcagaaaggtaacctcctccgccacattaaaCTG | 46L_47W_49P_50I_52G_53F_54S_55C_67L | 3.311146962 |
| 991 | 4315 | TTCTGGTGTCCCGTATGTGGGGCATCTTTTactcagaaaggtaacctcctccgccacattaaaCTG | 46F 47W 49P_50V 52G 53A 54S 55F 67L | 3.308069023 |
| 992 | 3421 | TTCAGCTGTGAAATCTGTGGGGCATGCTTTactcagaaaggtaacctcctccgccacattaaaCTG | 46F_47S_49E_50I_52G_53A_54C_55F_67L | 3.307298973 |
| 993 | 961 | TTCTTTTGTGATATCTGTGGGGCATGTTTTactcagaaaggtaacctcctccgccacattaaaCTG | 46F_47F_49D_50I_52G_53A_54C_55F 67L | 3.305997139 |
| 994 | 554 | TTCGATTGTGAGATTTGTGGGGCACGGTTTactcagaaaggtaacctcctccgccacattaaaCTG | 46F_47D_49E_50I_52G_53A_54R_55F_67L | 3.301492133 |
| 995 | 7977 | CTGTACTGCGAAATTTGCGGCTTTGTGTGTactcagaaaggtaacctcctccgccacattaaaCTG | 46L_47Y_49E_50I_52G_53F_54V_55C_67L | 3.301473932 |
| 996 | 4265 | TTCTGGTGTCCAATCTGTGGGGCAGGTTTTactcagaaaggtaacctcctccgccacattaaaCTG | 46F_47W_49P_50I_52G_53A_54G_55F_67L | 3.299514546 |
| 997 | 70 | TTCGCCTGTGAGATTTGTGGGGCAATGTTTactcagaaaggtaacctcctccgccacattaaaCTG | 46F_47A_49E_50I_52G_53A_54M_55F_67L | 3.296836235 |
| 998 | 77 | TTCGCGTGTGAGATCTGTGGGGCAGTGTTTactcagaaaggtaacctcctccgccacattaaaCTG | 46F_47A_49E_50I_52G_53A_54V_55F_67L | 3.294525569 |
| 999 | 7948 | CTGTGGTGCGAAATCTGCGGATTCAAGTGTactcagaaaggtaacctcctccgccacattaaaCTG | 46L_47W_49E_50I_52G_53F_54K_55C_67L | 3.29228761 |
| 1000 | 3352 | TTCCGCTGTCCCGTTTGTGGGGCACCATTTactcagaaaggtaacctcctccgccacattaaaCTG | 46F_47R_49P_50V_52G_53A_54P_55F_67L | 3.292180604 |
| 1001 | 4275 | TTCTGGTGTCCGATCTGTGGGGCAAGTTTTactcagaaaggtaacctcctccgccacattaaaCTG | 46F_47W_49P_50I_52G_53A_54S_55F_67L | 3.289541545 |
| 1002 | 6837 | CTATACTGCCCGATATGCGGGTTTGTCTGTactcagaaaggtaacctcctccgccacattaaaCTG | 46L_47Y_49P_50I_52G_53F_54V_55C_67L | 3.289108651 |
| 1003 | 4389 | TTCTACTGTGAAATCTGTGGGGCACTGTTTactcagaaaggtaacctcctccgccacattaaaCTG | 46F_47Y_49E_50I_52G_53A_54L 55F_67L | 3.288769181 |
| 1004 | 3552 | TTCTCATGTCCCATCTGTGGGGCACCGTTTactcagaaaggtaacctcctccgccacattaaaCTG | 46F_47S_49P_50I_52G_53A_54P_55F_67L | 3.28650892 |
| 1005 | 4272 | TTCTGGTGTCCCATCTGTGGGGCACCGTTTactcagaaaggtaacctcctccgccacattaaaCTG | 46F_47W_49P_50I_52G_53A_54P 55F_67L | 3.282617062 |
| 1006 | 1385 | TTCGGGTGTCCAATCTGTGGGGCAGGCTTTactcagaaaggtaacctcctccgccacattaaaCTG | 46F_47G_49P_50I_52G_53A_54G_55F_67L | 3.282060791 |
| 1007 | 546 | TTCGACTGTGAAATCTGTGGGGCACATTTTactcagaaaggtaacctcctccgccacattaaaCTG | 46F_47D_49E_50I 52G_53A 54H 55F 67L | 3.278795498 |
| 1008 | 4095 | TTCTGGTGTGATATCTGTGGGGCAAGTTTTactcagaaaggtaacctcctccgccacattaaaCTG | 46F 47W_49D_50I_52G_53A 54S 55F 67L | 3.27785183 |
| 1009 | 7697 | CTGTTCTGCGAGATTTGCGGATTTGTCTGCactcagaaaggtaacctcctccgccacattaaaCTG | 46L 47F 49E 50I 52G_53F 54V 55C_67L | 3.274657207 |
| 1010 | 1036 | TTCTTTTGTGAAATTTGTGGGGCAACTTTTactcagaaaggtaacctcctccgccacattaaaCTG | 46F_47F_49E_50I_52G_53A 54T 55F 67L | 3.273418452 |
| 1011 | 1395 | TTCGGCTGTCCCATCTGTGGGGCATCATTTactcagaaaggtaacctcctccgccacattaaaCTG | 46F_47G_49P_50I_52G_53A_54S_55F_67L | 3.271726874 |
| 1012 | 6564 | TTAGGCTGTCCGATTTGTGGCTTCTTTTGCactcagaaaggtaacctcctccgccacattaaaCTG | 46L_47G_49P_50I_52G_53F_54F_55C_67L | 3.269484904 |
| 1013 | 3461 | TTCAGCTGTGAAGTCTGTGGGGCATGCTTTactcagaaaggtaacctcctccgccacattaaaCTG | 46F_47S_49E_50V_52G_53A 54C_55F_67L | 3.26470381 |
| 1014 | 4519 | TTCTACTGTCCAATATGTGGGGCATACTTTactcagaaaggtaacctcctccgccacattaaaCTG | 46F_47Y_49P_50I_52G_53A_54Y_55F_67L | 3.261978068 |
| 1015 | 4431 | TTCTATTGTGAGGTATGTGGGGCAAACTTTactcagaaaggtaacctcctccgccacattaaaCTG | 46F_47Y_49E_50V_52G_53A_54N_55F_67L | 3.261421843 |
| 1016 | 1155 | TTCTTCTGTCCCATCTGTGGGGCATCCTTTactcagaaaggtaacctcctccgccacattaaaCTG | 46F_47F_49P_50I_52G_53A_54S_55F_67L | 3.260574214 |
| 1017 | 3912 | TTCGTGTGTGAAATCTGTGGGGCACCCTTTactcagaaaggtaacctcctccgccacattaaaCTG | 46F_47V_49E_50I_52G_53A_54P_55F_67L | 3.260545061 |
| 1018 | 1425 | TTCGGGTGTCCTGTTTGTGGGGCAGGCTTTactcagaaaggtaacctcctccgccacattaaaCTG | 46F_47G_49P_50V_52G_53A_54G_55F_67L | 3.259100377 |
| 1019 | 4541 | TTCTACTGTCCGGTTTGTGGGGCATGCTTTactcagaaaggtaacctcctccgccacattaaaCTG | 46F_47Y_49P_50V_52G_53A_54C_55F_67L | 3.255640413 |
| 1020 | 314 | TTCTGCTGTGAAATATGTGGGGCACGTTTTactcagaaaggtaacctcctccgccacattaaaCTG | 46F_47C_49E_50I_52G_53A_54R_55F_67L | 3.252474124 |
| 1021 | 110 | TTCGCATGTGAGGTGTGTGGGGCAATGTTTactcagaaaggtaacctcctccgccacattaaaCTG | 46F_47A_49E_50V_52G_53A_54M_55F_67L | 3.250602812 |
| 1022 | 4399 | TTCTACTGTGAGATCTGTGGGGCATACTTTactcagaaaggtaacctcctccgccacattaaaCTG | 46F_47Y_49E_50I_52G_53A_54Y_55F_67L | 3.249761988 |
| 1023 | 3434 | TTCTCGTGTGAAATTTGTGGGGCACGTTTTactcagaaaggtaacctcctccgccacattaaaCTG | 46F_47S_49E_50I_52G_53A 54R_55F 67L | 3.249323057 |
| 1024 | 4515 | TTCTACTGTCCGATCTGTGGGGCAAGTTTTactcagaaaggtaacctcctccgccacattaaaCTG | 46F_47Y_49P_50I_52G_53A_54S_55F_67L | 3.246636521 |
| 1025 | 117 | TTCGCCTGTGAGGTGTGTGGGGCAGTCTTTactcagaaaggtaacctcctccgccacattaaaCTG | 46F_47A_49E_50V_52G_53A 54V 55F_67L | 3.242931429 |
| 1026 | 6554 | CTGTTTTGCCCTATTTGTGGATTCCGCTGCactcagaaaggtaacctcctccgccacattaaaCTG | 46L_47F_49P_50I_52G_53F_54R_55C_67L | 3.242116398 |
| 1027 | 6816 | CTGTGGTGTCCCATCTGCGGCTTCACTTGTactcagaaaggtaacctcctccgccacattaaaCTG | 46L_47W_49P_50I_52G_53F_54T_55C_67L | 3.242111228 |
| 1028 | 4159 | TTCTGGTGTGAAATTTGTGGGGCATACTTTactcagaaaggtaacctcctccgccacattaaaCTG | 46F_47W_49E_50I_52G_53A_54Y_55F_67L | 3.239161312 |
| 1029 | 1500 | TTCCACTGTGAAATATGTGGGGCAGCGTTTactcagaaaggtaacctcctccgccacattaaaCTG | 46F_47H_49E_50I_52G_53A_54A 55F_67L | 3.237957018 |
| 1030 | 975 | TTCTTCTGTGACATCTGTGGGGCAAGTTTTactcagaaaggtaacctcctccgccacattaaaCTG | 46F_47F_49D_50I_52G_53A_54S_55F 67L | 3.235228849 |
| 1031 | 3428 | TTCTCCTGTGAAATCTGTGGGGCAAAATTTactcagaaaggtaacctcctccgccacattaaaCTG | 46F_47S_49E_50I_52G_53A_54K_55F_67L | 3.233474302 |
| 1032 | 4312 | TTCTGGTGTCCTGTGTGTGGGGCACCATTTactcagaaaggtaacctcctccgccacattaaaCTG | 46F_47W_49P_50V_52G_53A_54P_55F_67L | 3.2314539 |
| 1033 | 236 | TTCGCTTGTCCCGTCTGTGGGGCAACATTTactcagaaaggtaacctcctccgccacattaaaCTG | 46F_47A_49P_50V_52G_53A_54T_55F_67L | 3.229065452 |
| 1034 | 348 | TTCTGCTGTGAGGTCTGTGGGGCAAAATTTactcagaaaggtaacctcctccgccacattaaaCTG | 46F_47C_49E_50V_52G_53A_54K 55F 67L | 3.226295171 |
| 1035 | 3580 | TTCAGTTGTCCCGTCTGTGGGGCAGCCTTTactcagaaaggtaacctcctccgccacattaaaCTG | 46F_47S_49P_50V_52G_53A_54A_55F 67L | 3.225418219 |
| 1036 | 352 | TTCTGTTGTGAGGTTTGTGGGGCACCCTTTactcagaaaggtaacctcctcogccacattaaaCTG | 46F_47C_49E_50V_52G_53A_54P_55F_67L | 3.225088548 |
| 1037 | 1276 | TTCGGATGTGAGATCTGTGGGGCAACGTTTactcagaaaggtaacctcctccgccacattaaaCTG | 46F_47G_49E_50I_52G_53A 54T 55F 67L | 3.224891355 |
| 1038 | 4185 | TTCTGGTGTGAAGTGTGTGGGGCAGGATTTactcagaaaggtaacctcctccgccacattaaaCTG | 46F_47W_49E_50V_52G_53A_54G_55F_67L | 3.224058428 |
| 1039 | 3465 | TTCTCTTGTGAAGTGTGTGGGGCAGGATTTactcagaaaggtaacctcctccgccacattaaaCTG | 46F 47S 49E 50V 52G 53A 54G 55F 67L | 3.223385909 |
| 1040 | 7688 | TTATTTTGCGAGATTTGTGGGTTTAAATGCactcagaaaggtaacctcctccgccacattaaaCTG | 46L_47F_49E_50I_52G_53F_54K_55C_67L | 3.22205054 |
| 1041 | 1141 | TTCTTTTGTCCGATTTGTGGGGCATGCTTTactcagaaaggtaacctcctccgccacattaaaCTG | 46F 47F 49P 50I 52G 53A 54C 55F 67L | 3.220327078 |
| 1042 | 4088 | TTCTGGTGTGATATTTGTGGGGCAAAGTTTactcagaaaggtaacctcctccgccacattaaaCTG | 46F_47W_49D_50I_52G_53A_54K_55F 67L | 3.219208152 |
| 1043 | 6839 | CTCTATTGTCCAATCTGCGGTTTTTATTGCactcagaaaggtaacctcctccgccacattaaaCTG | 46L_47Y_49P_50I_52G_53F_54Y_55C_67L | 3.21882123 |
| 1044 | 2755 | TTCAATTGTGAGGTGTGTGGGGCAAGCTTTactcagaaaggtaacctcctccgccacattaaaCTG | 46F_47N_49E_50V_52G_53A_54S_55F_67L | 3.217478933 |
| 1045 | 4374 | TTCTACTGTGACGTGTGTGGGGCACGTTTTactcagaaaggtaacctcctccgccacattaaaCTG | 46F_47Y_49D_50V_52G_53A_54R_55F_67L | 3.210467802 |
| 1046 | 6813 | CTGTGGTGTCCGATCTGCGGCTTTCAATGCactcagaaaggtaacctcctccgccacattaaaCTG | 46L_47W_49P_50I_52G_53F_54Q_55C_67L | 3.208441257 |
| 1047 | 4328 | TTCTACTGTGATATTTGTGGGGCAAAGTTTactcagaaaggtaacctcctccgccacattaaaCTG | 46F_47Y_49D_50I_52G_53A_54K_55F_67L | 3.204914091 |
| 1048 | 4516 | TTCTATTGTCCAATCTGTGGGGCAACATTTactcagaaaggtaacctcctccgccacattaaaCTG | 46F_47Y_49P_50I_52G_53A_54T_55F_67L | 3.202460024 |
| 1049 | 7690 | TTATTCTGTGAGATCTGTGGATTTATGTGCactcagaaaggtaacctcctccgccacattaaaCTG | 46L_47F_49E_50I_52G_53F_54M_55C_67L | 3.200917049 |
| 1050 | 1672 | TTCCATTGTCCAGTGTGTGGGGCACCCTTTactcagaaaggtaacctcctccgccacattaaaCTG | 46F_47H_49P_50V_52G_53A_54P_55F 67L | 3.200869802 |
| 1051 | 4513 | TTCTACTGTCCTATCTGTGGGGCACAGTTTactcagaaaggtaacctcctccgccacattaaaCTG | 46F_47Y_49P_50I_52G_53A 54Q_55F_67L | 3.199550155 |
| 1052 | 6578 | CTGGGGTGCCCAATCTGTGGCTTTTGGTGCactcagaaaggtaacctcctccgccacattaaaCTG | 46L_47G_49P_50I_52G_53F_54W_55C_67L | 3.19855737 |
| 1053 | 6190 | TTTGGGTGCCCAATCTGTGGTCGAATGTTCactcagaaaggtaacctcctccgccacattaaaCTG | 46F_47G_49P_50I_52G_53R_54M_55F_67L | 3.19754793 |
| 1054 | 7953 | CTGTGGTGTGAGATTTGTGGCTTCCAGTGCactcagaaaggtaacctcctccgccacattaaaCTG | 46L_47W_49E_50I_52G_53F_54Q_55C_67L | 3.19749327 |
| 1055 | 6801 | CTCTGGTGTCCCATTTGTGGCTTTTGCTGCactcagaaaggtaacctcctccgccacattaaaCTG | 46L_47W_49P_50I_52G_53F 54C 55C 67L | 3.195892722 |
| 1056 | 7735 | CTGCACTGTGAAATATGCGGCTTTTCATGCactcagaaaggtaacctcctccgccacattaaaCTG | 46L_47H_49E_50I_52G_53F_54S_55C_67L | 3.193610823 |
| 1057 | 112 | TTCGCTTGTGAAGTCTGTGGGGCACCCTTTactcagaaaggtaacctcctccgccacattaaaCTG | 46F_47A_49E_50V_52G_53A_54P_55F 67L | 3.192484788 |
| 1058 | 1023 | TTCTTCTGTGAAATATGTGGGGCAGAGTTTactcagaaaggtaacctcctccgccacattaaaCTG | 46F_47F_49E_50I_52G_53A_54E_55F_67L | 3.192443274 |
| 1059 | 4335 | TTCTACTGTGATATTTGTGGGGCAAGCTTTactcagaaaggtaacctcctccgccacattaaaCTG | 46F_47Y_49D_50I_52G_53A_54S 55F 67L | 3.189382579 |
| 1060 | 4511 | TTCTATTGTCCCATTTGTGGGGCAAACTTTactcagaaaggtaacctcctccgccacattaaaCTG | 46F_47Y_49P_50I_52G_53A_54N_55F_67L | 3.187376578 |
| 1061 | 4325 | TTCTATTGTGATATCTGTGGGGCAGGGTTTactcagaaaggtaacctcctccgccacattaaaCTG | 46F_47Y_49D_50I_52G_53A_54G_55F_67L | 3.187279363 |
| 1062 | 1313 | TTCGGTTGTGAGGTTTGTGGGGCACAGTTTactcagaaaggtaacctcctccgccacattaaaCTG | 46F_47G_49E_50V_52G_53A_54Q_55F_67L | 3.182491513 |
| 1063 | 1545 | TTCCATTGTGAGGTCTGTGGGGCAGGCTTTactcagaaaggtaacctcctccgccacattaaaCTG | 46F_47H_49E_50V_52G_53A_54G 55F 67L | 3.182343627 |
| 1064 | 4429 | TTCTATTGTGAAGTGTGTGGGGCACTGTTTactcagaaaggtaacctcctccgccacattaaaCTG | 46F 47Y 49E_50V_52G_53A 54L 55F_67L | 3.182119105 |
| 1065 | 313 | TTCTGTTGTGAGATATGTGGGGCACAATTTactcagaaaggtaacctcctccgccacattaaaCTG | 46F_47C_49E_50I_52G_53A_54Q_55F_67L | 3.182005411 |
| 1066 | 1029 | TTCTTCTGTGAAATTTGTGGGGCACTGTTTactcagaaaggtaacctcctccgccacattaaaCTG | 46F_47F_49E_50I_52G_53A_54L_55F_67L | 3.181829928 |
| 1067 | 555 | TTCGATTGTGAGATTTGTGGGGCATCCTTTactcagaaaggtaacctcctccgccacattaaaCTG | 46F_47D_49E_50I_52G_53A_54S_55F_67L | 3.180566574 |
| 1068 | 4333 | TTCTACTGTGACATTTGTGGGGCACAGTTTactcagaaaggtaacctcctccgccacattaaaCTG | 46F_47Y_49D_50I_52G_53A_54Q_55F_67L | 3.178638901 |
| 1069 | 7680 | TTGTTTTGTGAGATCTGTGGTTTTGCGTGCactcagaaaggtaacctcctccgccacattaaaCTG | 46L_47F_49E_50I_52G_53F_54A_55C_67L | 3.177198006 |
| 1070 | 1452 | TTCCATTGTGACATCTGTGGGGCACCGTTTactcagaaaggtaacctcctccgccacattaaaCTG | 46F_47H_49D_50I_52G_53A_54P_55F_67L | 3.176147357 |
| 1071 | 6574 | CTGGGCTGCCCCATCTGCGGTTTCCGTTGCactcagaaaggtaacctcctccgccacattaaaCTG | 46L_47G_49P_50I_52G_53F_54R_55C_67L | 3.174540394 |
| 1072 | 79 | TTCGCATGTGAGATCTGTGGGGCATACTTTactcagaaaggtaacctcctccgccacattaaaCTG | 46F_47A_49E_50I_52G_53A_54Y_55F_67L | 3.171592439 |
| 1073 | 7960 | CTGTACTGTGAGATTTGCGGATTCGCTTGTactcagaaaggtaacctcctccgccacattaaaCTG | 46L_47Y_49E_50I_52G_53F_54A_55C_67L | 3.171186725 |
| 1074 | 6561 | CTGGGCTGCCCCATCTGCGGTTTCTGCTGCactcagaaaggtaacctcctccgccacattaaaCTG | 46L_47G_49P_50I_52G_53F_54C_55C_67L | 3.170260019 |
| 1075 | 1384 | TTCGGCTGTCCTATCTGTGGGGCATTTTTTactcagaaaggtaacctcctccgccacattaaaCTG | 46F_47G_49P_50I_52G_53A 54F_55F_67L | 3.169565756 |
| 1076 | 3132 | TTCAGGTGTGATATCTGTGGGGCACCCTTTactcagaaaggtaacctcctccgccacattaaaCTG | 46F_47R_49D_50I_52G_53A_54P_55F_67L | 3.168928038 |
| 1077 | 4147 | TTCTGGTGTGAGATTTGTGGGGCAATATTTactcagaaaggtaacctcctccgccacattaaaCTG | 46F_47W_49E_50I_52G_53A_54I_55F_67L | 3.168663124 |
| 1078 | 106 | TTCGCCTGTGAGGTGTGTGGGGCACACTTTactcagaaaggtaacctcctccgccacattaaaCTG | 46F_47A_49E_50V_52G_53A 54H 55F 67L | 3.161908778 |
| 1079 | 7613 | CTGGCTTGCGAGATATGCGGTTTTCAGTGTactcagaaaggtaacctcctccgccacattaaaCTG | 46L_47A_49E_50I_52G_53F_54Q_55C_67L | 3.161633212 |
| 1080 | 4503 | TTCTACTGTCCGATTTGTGGGGCAGAATTTactcagaaaggtaacctcctccgccacattaaaCTG | 46F_47Y_49P_50I_52G_53A_54E_55F_67L | 3.161161527 |
| 1081 | 3660 | TTCACGTGTGAAATTTGTGGGGCAGCTTTTactcagaaaggtaacctcctccgccacattaaaCTG | 46F_47T_49E_50I_52G_53A_54A_55F_67L | 3.156226091 |
| 1082 | 460 | TTCTGTTGTCCTGTGTGTGGGGCAGCCTTTactcagaaaggtaacctcctccgccacattaaaCTG | 46F_47C_49P_50V_52G_53A_54A_55F_67L | 3.155278489 |
| 1083 | 1274 | TTCGGCTGTGAGATTTGTGGGGCAAGATTTactcagaaaggtaacctcctccgccacattaaaCTG | 46F_47G_49E_50I_52G_53A_54R_55F_67L | 3.153259958 |
| 1084 | 4094 | TTCTGGTGTGATATCTGTGGGGCACGCTTTactcagaaaggtaacctcctccgccacattaaaCTG | 46F_47W_49D_50I_52G_53A_54R_55F_67L | 3.149478335 |
| 1085 | 2745 | TTCAACTGTGAGGTATGTGGGGCAGGTTTTactcagaaaggtaacctcctccgccacattaaaCTG | 46F_47N_49E_50V_52G_53A_54G_55F_67L | 3.149273248 |
| 1086 | 1185 | TTCTTCTGTCCTGTATGTGGGGCAGGTTTTactcagaaaggtaacctcctccgccacattaaaCTG | 46F_47F_49P_50V_52G_53A_54G_55F_67L | 3.14853251 |
| 1087 | 1140 | TTCTTCTGTCCAATTTGTGGGGCAGCCTTTactcagaaaggtaacctcctccgccacattaaaCTG | 46F_47F_49P_50I_52G_53A_54A_55F_67L | 3.1473906 |
| 1088 | 6189 | TTTGGATGTCCCATTTGCGGGAGACTGTTCactcagaaaggtaacctcctccgccacattaaaCTG | 46F_47G_49P_50I_52G_53R_54L_55F_67L | 3.145759742 |
| 1089 | 425 | TTCTGCTGTCCTATTTGTGGGGCAGGCTTTactcagaaaggtaacctcctccgccacattaaaCTG | 46F_47C_49P_50I_52G_53A_54G_55F_67L | 3.140179316 |
| 1090 | 6830 | TTATATTGTCCTATCTGTGGATTCATGTGCactcagaaaggtaacctcctccgccacattaaaCTG | 46L_47Y_49P_50I_52G_53F_54M_55C_67L | 3.138282279 |
| 1091 | 1070 | TTCTTTTGTGAAGTGTGTGGGGCAATGTTTactcagaaaggtaacctcctccgccacattaaaCTG | 46F_47F_49E_50V_52G_53A_54M 55F 67L | 3.137871234 |
| 1092 | 4190 | TTCTGGTGTGAAGTGTGTGGGGCAATGTTTactcagaaaggtaacctcctccgccacattaaaCTG | 46F_47W_49E_50V_52G_53A_54M_55F_67L | 3.135942695 |
| 1093 | 1429 | TTCGGATGTCCTGTCTGTGGGGCACTGTTTactcagaaaggtaacctcctccgccacattaaaCTG | 46F_47G_49P_50V_52G_53A_54L_55F_67L | 3.135834288 |
| 1094 | 7966 | CTGTACTGCGAGATCTGTGGCTTCCATTGTactcagaaaggtaacctcctccgccacattaaaCTG | 46L_47Y_49E_50I_52G_53F_54H_55C_67L | 3.134780592 |
| 1095 | 6756 | CTCAGCTGTCCTATTTGTGGATTTACCTGTactcagaaaggtaacctcctccgccacattaaaCTG | 46L_47S_49P_50I_52G_53F_54T_55C_67L | 3.131416066 |
| 1096 | 1515 | TTCCACTGTGAGATTTGTGGGGCATCTTTTactcagaaaggtaacctcctccgccacattaaaCTG | 46F_47H_49E_50I_52G_53A_54S_55F_67L | 3.130917127 |
| 1097 | 472 | TTCTGCTGTCCTGTTTGTGGGGCACCGTTTactcagaaaggtaacctcctccgccacattaaaCTG | 46F_47C_49P_50V_52G_53A_54P_55F_67L | 3.127205934 |
| 1098 | 6826 | CTCTACTGTCCCATCTGTGGCTTCCACTGCactcagaaaggtaacctcctccgccacattaaaCTG | 46L_47Y_49P_50I_52G_53F_54H_55C_67L | 3.127108468 |
| 1099 | 4300 | TTCTGGTGTCCAGTGTGTGGGGCAGCATTTactcagaaaggtaacctcctccgccacattaaaCTG | 46F_47W_49P_50V_52G_53A 54A 55F_67L | 3.126664945 |
| 1100 | 1154 | TTCTTTTGTCCAATCTGTGGGGCACGCTTTactcagaaaggtaacctcctccgccacattaaaCTG | 46F_47F_49P_50I_52G_53A_54R_55F_67L | 3.126409264 |
| 1101 | 1625 | TTCCACTGTCCTATTTGTGGGGCAGGATTTactcagaaaggtaacctcctccgccacattaaaCTG | 46F_47H_49P_50I_52G_53A_54G_55F_67L | 3.12332691 |
| 1102 | 4506 | TTCTACTGTCCGATCTGTGGGGCACATTTTactcagaaaggtaacctcctccgccacattaaaCTG | 46F_47Y_49P_50I_52G_53A_54H_55F_67L | 3.122490131 |
| 1103 | 4260 | TTCTGGTGTCCCATATGTGGGGCAGCTTTTactcagaaaggtaacctcctccgccacattaaaCTG | 46F_47W_49P_50I_52G_53A_54A_55F_67L | 3.120317692 |
| 1104 | 4080 | TTCTGGTGTGATATCTGTGGGGCAGCCTTTactcagaaaggtaacctcctccgccacattaaaCTG | 46F_47W_49D_50I_52G_53A_54A_55F_67L | 3.119808794 |
| 1105 | 3540 | TTCAGCTGTCCAATATGTGGGGCAGCTTTTactcagaaaggtaacctcctccgccacattaaaCTG | 46F_47S_49P_50I_52G_53A 54A 55F_67L | 3.118946833 |
| 1106 | 714 | TTCGACTGTCCAGTGTGTGGGGCACGGTTTactcagaaaggtaacctcctccgccacattaaaCTG | 46F_47D_49P_50V_52G_53A_54R_55F_67L | 3.11855867 |
| 1107 | 4426 | TTCTACTGTGAGGTGTGTGGGGCACATTTTactcagaaaggtaacctcctccgccacattaaaCTG | 46F_47Y_49E_50V_52G_53A_54H_55F_67L | 3.117511643 |
| 1108 | 8 | TTCGCCTGTGATATCTGTGGGGCAAAGTTTactcagaaaggtaacctcctccgccacattaaaCTG | 46F_47A_49D_50I_52G_53A_54K_55F_67L | 3.115205312 |
| 1109 | 3112 | TTCCAATGTCCCGTTTGTGGGGCACCTTTTactcagaaaggtaacctcctccgccacattaaaCTG | 46F 47Q 49P 50V 52G 53A 54P 55F 67L | 3.112641957 |
| 1110 | 1193 | TTCTTCTGTCCAGTGTGTGGGGCACAGTTTactcagaaaggtaacctcctccgccacattaaaCTG | 46F_47F_49P_50V_52G_53A_54Q_55F_67L | 3.108531799 |
| 1111 | 4436 | TTCTACTGTGAGGTTTGTGGGGCAACGTTTactcagaaaggtaacctcctccgccacattaaaCTG | 46F_47Y_49E_50V_52G_53A_54T_55F_67L | 3.103557503 |
| 1112 | 4186 | TTCTGGTGTGAGGTGTGTGGGGCACATTTTactcagaaaggtaacctcctccgccacattaaaCTG | 46F_47W_49E_50V_52G_53A_54H_55F_67L | 3.103458014 |
| 1113 | 6466 | TTGGCCTGTCCGATATGTGGTTTTCATTGCactcagaaaggtaacctcctccgccacattaaaCTG | 46L_47A_49P_50I_52G_53F_54H_55C_67L | 3.098120843 |
| 1114 | 6560 | CTGGGGTGCCCAATCTGCGGCTTTGCATGTactcagaaaggtaacctcctccgccacattaaaCTG | 46L_47G_49P_50I_52G_53F_54A_55C_67L | 3.09754978 |
| 1115 | 233 | TTCGCCTGTCCTGTTTGTGGGGCACAGTTTactcagaaaggtaacctcctccgccacattaaaCTG | 46F_47A_49P_50V_52G_53A_54Q_55F_67L | 3.096768157 |
| 1116 | 7600 | CTTGCCTGCGAGATATGCGGCTTTGCCTGTactcagaaaggtaacctcctccgccacattaaaCTG | 46L 47A_49E_50I_52G_53F_54A_55C_67L | 3.09543747 |
| 1117 | 594 | TTCGACTGTGAGGTATGTGGGGCAAGGTTTactcagaaaggtaacctcctccgccacattaaaCTG | 46F_47D_49E_50V_52G_53A_54R_55F_67L | 3.093345067 |
| 1118 | 237 | TTCGCCTGTCCCGTCTGTGGGGCAGTGTTTactcagaaaggtaacctcctccgccacattaaaCTG | 46F_47A_49P_50V_52G_53A_54V_55F_67L | 3.092641988 |
| 1119 | 4372 | TTCTACTGTGATGTCTGTGGGGCACCGTTTactcagaaaggtaacctcctccgccacattaaaCTG | 46F_47Y_49D_50V_52G_53A_54P_55F_67L | 3.088616668 |
| 1120 | 1316 | TTCGGTTGTGAAGTCTGTGGGGCAACCTTTactcagaaaggtaacctcctccgccacattaaaCTG | 46F_47G_49E_50V_52G_53A_54T_55F_67L | 3.088176288 |
| 1121 | 4555 | TTCTACTGTCCCGTGTGTGGGGCAAGCTTTactcagaaaggtaacctcctccgccacattaaaCTG | 46F_47Y_49P_50V_52G_53A_54S_55F_67L | 3.086000618 |
| 1122 | 1039 | TTCTTTTGTGAAATATGTGGGGCATATTTTactcagaaaggtaacctcctccgccacattaaaCTG | 46F_47F_49E_50I_52G_53A_54Y_55F_67L | 3.084611048 |
| 1123 | 4504 | TTCTACTGTCCAATCTGTGGGGCATTTTTTactcagaaaggtaacctcctccgccacattaaaCTG | 46F_47Y_49P_50I_52G_53A_54F_55F_67L | 3.084394365 |
| 1124 | 186 | TTCGCCTGTCCCATATGTGGGGCACACTTTactcagaaaggtaacctcctccgccacattaaaCTG | 46F_47A_49P_50I_52G_53A_54H_55F_67L | 3.083310908 |
| 1125 | 2700 | TTCAATTGTGAAATCTGTGGGGCAGCATTTactcagaaaggtaacctcctccgccacattaaaCTG | 46F_47N_49E_50I_52G_53A_54A_55F_67L | 3.080999776 |
| 1126 | 1540 | TTCCATTGTGAAGTGTGTGGGGCAGCCTTTactcagaaaggtaacctcctccgccacattaaaCTG | 46F_47H_49E_50V_52G_53A_54A_55F_67L | 3.080348557 |
| 1127 | 4192 | TTCTGGTGTGAGGTGTGTGGGGCACCCTTTactcagaaaggtaacctcctccgccacattaaaCTG | 46F_47W_49E_50V_52G_53A_54P_55F_67L | 3.079301566 |
| 1128 | 3468 | TTCTCATGTGAAGTGTGTGGGGCAAAGTTTactcagaaaggtaacctcctccgccacattaaaCTG | 46F_47S_49E_50V_52G_53A_54K_55F_67L | 3.076916959 |
| 1129 | 6834 | CTTTATTGTCCCATCTGTGGATTTCGGTGTactcagaaaggtaacctcctccgccacattaaaCTG | 46L_47Y_49P_50I_52G_53F_54R_55C_67L | 3.076328826 |
| 1130 | 1031 | TTCTTCTGTGAGATTTGTGGGGCAAATTTTactcagaaaggtaacctcctccgccacattaaaCTG | 46F_47F_49E_50I_52G_53A_54N_55F_67L | 3.075299287 |
| 1131 | 1275 | TTCGGATGTGAGATCTGTGGGGCATCCTTTactcagaaaggtaacctcctccgccacattaaaCTG | 46F_47G_49E_50I_52G_53A_54S_55F_67L | 3.071447191 |
| 1132 | 475 | TTCTGTTGTCCAGTCTGTGGGGCATCCTTTactcagaaaggtaacctcctccgccacattaaaCTG | 46F_47C_49P_50V_52G_53A_54S_55F_67L | 3.071204275 |
| 1133 | 1153 | TTCTTTTGTCCTATTTGTGGGGCACAGTTTactcagaaaggtaacctcctccgccacattaaaCTG | 46F_47F_49P_50I_52G_53A_54Q_55F67L_ | 3.07015378 |
| 1134 | 2740 | TTCAATTGTGAAGTGTGTGGGGCAGCGTTTactcagaaaggtaacctcctccgccacattaaaCTG | 46F_47N_49E_50V_52G_53A_54A_55F_67L | 3.06781668 |
| 1135 | 6188 | TTCGGATGCCCCATCTGTGGGCGAAAATTTactcagaaaggtaacctcctccgccacattaaaCTG | 46F_47G_49P_50I_52G_53R_54K_55F_67L | 3.060498607 |
| 1136 | 6545 | CTCTTCTGCCCAATCTGTGGCTTTGGATGTactcagaaaggtaacctcctccgccacattaaaCTG | 46L_47F_49P_50I_52G_53F_54G_55C_67L | 3.059512537 |
| 1137 | 15 | TTCGCCTGTGATATTTGTGGGGCAAGTTTTactcagaaaggtaacctcctccgccacattaaaCTG | 46F_47A_49D_50I_52G_53A_54S_55F_67L | 3.05945938 |
| 1138 | 6197 | TTCGGATGTCCAATTTGTGGGAGGGTGTTTactcagaaaggtaacctcctccgccacattaaaCTG | 46F_47G_49P_50I_52G_53R_54V_55F_67L | 3.057075296 |
| 1139 | 4311 | TTCTGGTGTCCCGTCTGTGGGGCAAATTTTactcagaaaggtaacctcctccgccacattaaaCTG | 46F_47W_49P_50V_52G_53A_54N_55F 67L | 3.052228852 |
| 1140 | 4156 | TTCTGGTGTGAAATCTGTGGGGCAACGTTTactcagaaaggtaacctcctccgccacattaaaCTG | 46F_47W_49E_50I_52G_53A_54T_55F_67L | 3.051679795 |
| 1141 | 1620 | TTCCATTGTCCAATATGTGGGGCAGCCTTTactcagaaaggtaacctcctccgccacattaaaCTG | 46F_47H_49P_50I_52G_53A_54A_55F_67L | 3.05103678 |
| 1142 | 7950 | CTCTGGTGTGAGATATGCGGGTTTATGTGCactcagaaaggtaacctcctccgccacattaaaCTG | 46L_47W_49E_50I_52G_53F_54M_55C_67L | 3.049569774 |
| 1143 | 2992 | TTCCAATGTGAGGTCTGTGGGGCACCATTTactcagaaaggtaacctcctccgccacattaaaCTG | 46F_47Q_49E_50V_52G_53A_54P_55F_67L | 3.048493128 |
| 1144 | 7896 | CTGTCCTGCGAAATCTGCGGATTCACATGCactcagaaaggtaacctcctccgccacattaaaCTG | 46L_47S_49E_50I_52G_53F_54T_55C_67L | 3.048019731 |
| 1145 | 6540 | CTGTTCTGCCCGATCTGCGGTTTCGCCTGCactcagaaaggtaacctcctccgccacattaaaCTG | 46L_47F_49P_50I_52G_53F_54A_55C_67L | 3.04668833 |
| 1146 | 354 | TTCTGTTGTGAGGTGTGTGGGGCACGTTTTactcagaaaggtaacctcctccgccacattaaaCTG | 46F_47C_49E_50V_52G_53A_54R_55F_67L | 3.045594502 |
| 1147 | 4545 | TTCTACTGTCCCGTCTGTGGGGCAGGATTTactcagaaaggtaacctcctccgccacattaaaCTG | 46F_47Y_49P_50V_52G_53A_54G_55F_67L | 3.045263659 |
| 1148 | 3592 | TTCAGCTGTCCTGTATGTGGGGCACCGTTTactcagaaaggtaacctcctccgccacattaaaCTG | 46F_47S_49P_50V_52G_53A_54P_55F_67L | 3.044838138 |
| 1149 | 2772 | TTCAATTGTAACATTTGTGGGGCACCGTTTactcagaaaggtaacctcctccgccacattaaaCTG | 46F_47N_49N_50I_52G_53A_54P_55F_67L | 3.043477787 |
| 1150 | 1423 | TTCGGCTGTCCAGTTTGTGGGGCAGAGTTTactcagaaaggtaacctcctccgccacattaaaCTG | 46F_47G_49P_50V_52G_53A_54E_55F_67L | 3.041077584 |
| 1151 | 1260 | TTCGGATGTGAGATCTGTGGGGCAGCGTTTactcagaaaggtaacctcctccgccacattaaaCTG | 46F_47G_49E_50I_52G_53A_54A_55F_67L | 3.041047179 |
| 1152 | 6755 | CTTTCGTGTCCCATTTGCGGTTTCTCATGCactcagaaaggtaacctcctccgccacattaaaCTG | 46L_47S_49P_50I_52G_53F_54S_55C_67L | 3.040908057 |
| 1153 | 675 | TTCGATTGTCCTATTTGTGGGGCATCCTTTactcagaaaggtaacctcctccgccacattaaaCTG | 46F_47D_49P_50I_52G_53A_54S_55F_67L | 3.040543676 |
| 1154 | 191 | TTCGCCTGTCCAATCTGTGGGGCAAATTTTactcagaaaggtaacctcctccgccacattaaaCTG | 46F_47A_49P_50I_52G_53A_54N_55F_67L | 3.039033145 |
| 1155 | 6461 | CTTGCTTGCCCAATTTGCGGGTTTTGTTGCactcagaaaggtaacctcctccgccacattaaaCTG | 46L_47A_49P_50I_52G_53F_54C_55C_67L | 3.038103111 |
| 1156 | 1195 | TTCTTCTGTCCCGTTTGTGGGGCATCCTTTactcagaaaggtaacctcctccgccacattaaaCTG | 46F_47F_49P_50V_52G_53A_54S_55F_67L | 3.036833476 |
| 1157 | 7592 | TTTTACTGCGAGATATGCGGTCGCCCCTTCactcagaaaggtaacctcctccgccacattaaaCTG | 46F_47Y_49E_50I_52G_53R_54P 55F_67L | 3.036121416 |
| 1158 | 6577 | CTAGGCTGCCCAATCTGTGGTTTCGTGTGCactcagaaaggtaacctcctccgccacattaaaCTG | 46L_47G_49P_50I_52G_53F_54V_55C_67L | 3.035421611 |
| 1159 | 4144 | TTCTGGTGTGAAATTTGTGGGGCATTCTTTactcagaaaggtaacctcctccgccacattaaaCTG | 46F_47W_49E_50I 52G_53A 54F 55F_67L | 3.031936956 |
| 1160 | 1145 | TTCTTTTGTCCCATCTGTGGGGCAGGATTTactcagaaaggtaacctcctccgccacattaaaCTG | 46F_47F_49P_50I_52G_53A_54G_55F_67L | 3.024380072 |
| 1161 | 1143 | TTCTTCTGTCCAATTTGTGGGGCAGAGTTTactcagaaaggtaacctcctccgccacattaaaCTG | 46F_47F_49P_50I_52G_53A_54E_55F_67L | 3.023559044 |
| 1162 | 6557 | CTGTTCTGTCCGATCTGTGGCTTCGTATGTactcagaaaggtaacctcctccgccacattaaaCTG | 46L 47F_49P_50I_52G_53F_54V_55C_67L | 3.022217605 |
| 1163 | 6814 | CTTTGGTGCCCAATATGTGGCTTCCGGTGCactcagaaaggtaacctcctccgccacattaaaCTG | 46L_47W_49P_50I_52G_53F_54R_55C_67L | 3.019596929 |
| 1164 | 4273 | TTCTGGTGTCCAATTTGTGGGGCACAGTTTactcagaaaggtaacctcctccgccacattaaaCTG | 46F_47W_49P_50I_52G_53A_54Q_55F_67L | 3.014620485 |
| 1165 | 1389 | TTCGGTTGTCCAATTTGTGGGGCACTGTTTactcagaaaggtaacctcctccgccacattaaaCTG | 46F_47G_49P_50I_52G_53A_54L 55F_67L | 3.01251568 |
| 1166 | 6567 | TTGGGGTGCCCTATATGCGGCTTTATCTGCactcagaaaggtaacctcctccgccacattaaaCTG | 46L_47G_49P_50I_52G_53F_54I 55C_67L | 3.011883184 |
| 1167 | 1063 | TTCTTCTGTGAGGTATGTGGGGCAGAGTTTactcagaaaggtaacctcctccgccacattaaaCTG | 46F_47F_49E_50V_52G_53A_54E_55F 67L | 3.010464372 |
| 1168 | 372 | TTCTGTTGTAACATATGTGGGGCACCTTTTactcagaaaggtaacctcctccgccacattaaaCTG | 46F_47C_49N_50I_52G_53A_54P_55F_67L | 3.007799236 |
| 1169 | 666 | TTCGACTGTCCCATTTGTGGGGCACATTTTactcagaaaggtaacctcctccgccacattaaaCTG | 46F_47D_49P_50I_52G_53A_54H_55F_67L | 3.007501258 |
| 1170 | 6192 | TTCGGATGTCCCATTTGTGGCAGGCCCTTTactcagaaaggtaacctcctccgccacattaaaCTG | 46F 47G 49P 50I 52G 53R 54P 55F 67L | 3.007426964 |
| 1171 | 7964 | CTGTACTGCGAAATCTGCGGGTTTTTTTGCactcagaaaggtaacctcctccgccacattaaaCTG | 46L_47Y_49E_50I_52G_53F_54F_55C_67L | 3.003273143 |
| 1172 | 1181 | TTCTTCTGTCCAGTATGTGGGGCATGCTTTactcagaaaggtaacctcctccgccacattaaaCTG | 46F_47F_49P_50V_52G_53A_54C_55F_67L | 3.000111032 |
| 1173 | 1270 | TTCGGATGTGAGATCTGTGGGGCAATGTTTactcagaaaggtaacctcctccgccacattaaaCTG | 46F 47G 49E 50I 52G 53A 54M 55F 67L | 2.999958905 |
| 1174 | 1439 | TTCGGATGTCCCGTCTGTGGGGCATACTTTactcagaaaggtaacctcctccgccacattaaaCTG | 46F_47G_49P_50V_52G_53A 54Y 55F_67L | 2.997318832 |
| 1175 | 132 | TTCGCCTGTAACATCTGTGGGGCACCCTTTactcagaaaggtaacctcctccgccacattaaaCTG | 46F_47A_49N_50I_52G_53A_54P 55F_67L | 2.995512413 |
| 1176 | 353 | TTCTGCTGTGAGGTGTGTGGGGCACAGTTTactcagaaaggtaacctcctccgccacattaaaCTG | 46F_47C_49E_50V_52G_53A_54Q_55F_67L | 2.995250636 |
| 1177 | 4505 | TTCTATTGTCCAATATGTGGGGCAGGTTTTactcagaaaggtaacctcctccgccacattaaaCTG | 46F 47Y 49P 50I 52G 53A 54G 55F 67L | 2.994588258 |
| 1178 | 4439 | TTCTACTGTGAAGTGTGTGGGGCATACTTTactcagaaaggtaacctcctccgccacattaaaCTG | 46F 47Y 49E_50V_52G_53A 54Y 55F 67L | 2.993447433 |
| 1179 | 4271 | TTCTGGTGTCCCATCTGTGGGGCAAACTTTactcagaaaggtaacctcctccgccacattaaaCTG | 46F_47W_49P_50I_52G_53A_54N_55F_67L | 2.991744196 |
| 1180 | 1508 | TTCCACTGTGAGATTTGTGGGGCAAAGTTTactcagaaaggtaacctcctccgccacattaaaCTG | 46F_47H_49E_50I_52G_53A_54K 55F_67L | 2.990871223 |
| 1181 | 1992 | TTCAAGTGTGAAATCTGTGGGGCACCCTTTactcagaaaggtaacctcctccgccacattaaaCTG | 46F_47K_49E_50I_52G_53A_54P_55F_67L | 2.98962285 |
| 1182 | 6468 | TTGGCTTGCCCCATTTGTGGTTTCAAGTGCactcagaaaggtaacctcctccgccacattaaaCTG | 46L 47A_49P_50I_52G_53F_54K_55C_67L | 2.988479011 |
| 1183 | 3672 | TTCACGTGTGAGATCTGTGGGGCACCCTTTactcagaaaggtaacctcctccgccacattaaaCTG | 46F 47T 49E 50I 52G 53A 54P 55F 67L | 2.987994366 |
| 1184 | 7967 | CTCTACTGCGAGATCTGTGGGTTCATATGCactcagaaaggtaacctcctccgccacattaaaCTG | 46L_47Y_49E_50I_52G_53F_54I 55C_67L | 2.98734482 |
| 1185 | 6820 | CTCTACTGTCCGATCTGTGGCTTTGCTTGTactcagaaaggtaacctcctccgccacattaaaCTG | 46L_47Y_49P_50I_52G_53F_54A_55C_67L | 2.980714704 |
| 1186 | 4313 | TTCTGGTGTCCAGTGTGTGGGGCACAGTTTactcagaaaggtaacctcctccgccacattaaaCTG | 46F_47W_49P_50V_52G_53A_54Q_55F_67L | 2.977631768 |
| 1187 | 6465 | CTCGCATGCCCAATCTGTGGCTTTGGGTGCactcagaaaggtaacctcctccgccacattaaaCTG | 46L_47A_49P_50I_52G_53F_54G_55C_67L | 2.9729912 |
| 1188 | 586 | TTCGACTGTGAGGTGTGTGGGGCACACTTTactcagaaaggtaacctcctccgccacattaaaCTG | 46F_47D_49E_50V_52G_53A_54H_55F_67L | 2.97295546 |
| 1189 | 4143 | TTCTGGTGTGAGATTTGTGGGGCAGAGTTTactcagaaaggtaacctcctccgccacattaaaCTG | 46F_47W_49E_50I_52G_53A_54E_55F_67L | 2.971818363 |
| 1190 | 1501 | TTCCATTGTGAGATTTGTGGGGCATGCTTTactcagaaaggtaacctcctccgccacattaaaCTG | 46F_47H_49E_50I_52G_53A_54C_55F_67L | 2.970281633 |
| 1191 | 6571 | CTGGGCTGCCCCATATGTGGGTTCAACTGCactcagaaaggtaacctcctccgccacattaaaCTG | 46L 47G 49P 50I_52G 53F 54N_55C_67L | 2.96454861 |
| 1192 | 1012 | TTCTTCTGTGATGTTTGTGGGGCACCTTTTactcagaaaggtaacctcctccgccacattaaaCTG | 46F_47F_49D_50V_52G_53A_54P_55F 67L | 2.961893489 |
| 1193 | 465 | TTCTGCTGTCCTGTGTGTGGGGCAGGTTTTactcagaaaggtaacctcctccgccacattaaaCTG | 46F_47C_49P_50V_52G_53A_54G 55F 67L | 2.960267965 |
| 1194 | 1660 | TTCCACTGTCCTGTTTGTGGGGCAGCATTTactcagaaaggtaacctcctccgccacattaaaCTG | 46F_47H_49P_50V_52G_53A_54A_55F_67L | 2.95829778 |
| 1195 | 2232 | TTCCTGTGTGAAATCTGTGGGGCACCATTTactcagaaaggtaacctcctccgccacattaaaCTG | 46F 47L 49E_50I_52G_53A_54P 55F 67L | 2.956359935 |
| 1196 | 2820 | TTCAATTGTCCTATATGTGGGGCAGCCTTTactcagaaaggtaacctcctccgccacattaaaCTG | 46F_47N_49P_50I_52G_53A_54A 55F 67L | 2.956212747 |
| 1197 | 4201 | TTCTGGTGTAACATCTGTGGGGCATGTTTTactcagaaaggtaacctcctccgccacattaaaCTG | 46F_47W_49N_50I_52G_53A_54C_55F_67L | 2.954843673 |
| 1198 | 4492 | TTCTACTGTAATGTATGTGGGGCACCGTTTactcagaaaggtaacctcctccgccacattaaaCTG | 46F_47Y_49N_50V_52G_53A_54P_55F_67L | 2.950240496 |
| 1199 | 6478 | CTCGCCTGCCCGATTTGCGGGTTCTGGTGCactcagaaaggtaacctcctccgccacattaaaCTG | 46L_47A_49P_50I_52G_53F_54W_55C_67L | 2.948007112 |
| 1200 | 1424 | TTCGGCTGTCCCGTGTGTGGGGCATTTTTTactcagaaaggtaacctcctccgccacattaaaCTG | 46F_47G_49P_50V_52G_53A_54F_55F 67L | 2.947184188 |
| 1201 | 6800 | CTATGGTGCCCCATCTGCGGCTTTGCCTGCactcagaaaggtaacctcctccgccacattaaaCTG | 46L_47W_49P_50I_52G_53F_54A_55C_67L | 2.94539283 |
| 1202 | 6827 | TTATACTGTCCAATTTGTGGTTTTATTTGCactcagaaaggtaacctcctccgccacattaaaCTG | 46L_47Y_49P_50I_52G_53F_54I_55C_67L | 2.943791454 |
| 1203 | 6732 | CTGAGATGTCCTATTTGTGGCTTTCCCTGCactcagaaaggtaacctcctccgccacattaaaCTG | 46L_47R_49P_50I_52G_53F_54P_55C_67L | 2.943736453 |
| 1204 | 134 | TTCGCCTGTAACATTTGTGGGGCAAGGTTTactcagaaaggtaacctcctccgccacattaaaCTG | 46F_47A_49N_50I_52G_53A_54R_55F_67L | 2.943212563 |
| 1205 | 111 | TTCGCATGTGAAGTTTGTGGGGCAAATTTTactcagaaaggtaacctcctccgccacattaaaCTG | 46F_47A_49E_50V_52G_53A_54N_55F_67L | 2.940452582 |
| 1206 | 4268 | TTCTGGTGTCCCATCTGTGGGGCAAAGTTTactcagaaaggtaacctcctccgccacattaaaCTG | 46F_47W_49P_50I_52G_53A_54K_55F_67L | 2.937406284 |
| 1207 | 6546 | CTGTTTTGCCCCATCTGCGGCTTTCACTGTactcagaaaggtaacctcctccgccacattaaaCTG | 46L 47F 49P 50I_52G_53F 54H 55C_67L | 2.936287346 |
| 1208 | 71 | TTCGCTTGTGAAATCTGTGGGGCAAACTTTactcagaaaggtaacctcctccgccacattaaaCTG | 46F_47A_49E_50I_52G_53A_54N_55F_67L | 2.934297442 |
| 1209 | 1399 | TTCGGCTGTCCCATCTGTGGGGCATATTTTactcagaaaggtaacctcctccgccacattaaaCTG | 46F_47G_49P_50I_52G_53A_54Y 55F 67L | 2.933893751 |
| 1210 | 1555 | TTCCACTGTGAGGTGTGTGGGGCATCTTTTactcagaaaggtaacctcctccgccacattaaaCTG | 46F_47H_49E_50V_52G_53A_54S_55F_67L | 2.932227729 |
| 1211 | 1267 | TTCGGCTGTGAGATTTGTGGGGCAATTTTTactcagaaaggtaacctcctccgccacattaaaCTG | 46F 47G 49E 50I_52G_53A 54I 55F 67L | 2.932156393 |
| 1212 | 4149 | TTCTGGTGTGAAATCTGTGGGGCACTGTTTactcagaaaggtaacctcctccgccacattaaaCTG | 46F_47W_49E_50I_52G_53A_54L_55F_67L | 2.9309244 |
| 1213 | 4540 | TTCTACTGTCCAGTCTGTGGGGCAGCTTTTactcagaaaggtaacctcctccgccacattaaaCTG | 46F_47Y_49P_50V_52G_53A_54A 55F 67L | 2.929235048 |
| 1214 | 226 | TTCGCCTGTCCGGTATGTGGGGCACATTTTactcagaaaggtaacctcctccgccacattaaaCTG | 46F_47A_49P_50V_52G_53A_54H 55F 67L | 2.928760661 |
| 1215 | 6470 | CTCGCTTGTCCCATCTGCGGGTTCATGTGCactcagaaaggtaacctcctccgccacattaaaCTG | 46L_47A_49P_50I_52G_53F_54M_55C_67L | 2.926059645 |
| 1216 | 4337 | TTCTACTGTGATATATGTGGGGCAGTTTTTactcagaaaggtaacctcctccgccacattaaaCTG | 46F_47Y_49D_50I_52G_53A_54V_55F_67L | 2.924958318 |
| 1217 | 1148 | TTCTTTTGTCCCATATGTGGGGCAAAGTTTactcagaaaggtaacctcctccgccacattaaaCTG | 46F_47F_49P_50I_52G_53A_54K_55F_67L | 2.923391205 |
| 1218 | 4128 | TTCTGGTGTGATGTCTGTGGGGCAAAGTTTactcagaaaggtaacctcctccgccacattaaaCTG | 46F_47W_49D_50V_52G_53A 54K 55F 67L | 2.918122736 |
| 1219 | 960 | TTCTTCTGTGACATCTGTGGGGCAGCCTTTactcagaaaggtaacctcctccgccacattaaaCTG | 46F_47F_49D_50I_52G_53A_54A_55F_67L | 2.916697447 |
| 1220 | 1548 | TTCCATTGTGAAGTGTGTGGGGCAAAGTTTactcagaaaggtaacctcctccgccacattaaaCTG | 46F_47H_49E_50V_52G_53A_54K 55F 67L | 2.915334192 |
| 1221 | 197 | TTCGCCTGTCCCATCTGTGGGGCAGTATTTactcagaaaggtaacctcctccgccacattaaaCTG | 46F_47A_49P_50I_52G_53A_54V_55F_67L | 2.913371429 |
| 1222 | 4305 | TTCTGGTGTCCCGTGTGTGGGGCAGGTTTTactcagaaaggtaacctcctccgccacattaaaCTG | 46F_47W_49P_50V_52G_53A_54G_55F_67L | 2.909854234 |
| 1223 | 4092 | TTCTGGTGTGATATCTGTGGGGCACCCTTTactcagaaaggtaacctcctccgccacattaaaCTG | 46F_47W_49D_50I_52G_53A_54P_55F_67L | 2.905654942 |
| 1224 | 976 | TTCTTCTGTGACATTTGTGGGGCAACCTTTactcagaaaggtaacctcctccgccacattaaaCTG | 46F_47F_49D_50I_52G_53A 54T_55F 67L | 2.90530901 |
| 1225 | 7941 | CTATGGTGCGAAATATGCGGATTCTGTTGCactcagaaaggtaacctcctccgccacattaaaCTG | 46L_47W_49E_50I_52G_53F_54C_55C_67L | 2.902458402 |
| 1226 | 706 | TTCGATTGTCCTGTCTGTGGGGCACACTTTactcagaaaggtaacctcctccgccacattaaaCTG | 46F_47D_49P_50V_52G_53A_54H_55F_67L | 2.899660449 |
| 1227 | 2825 | TTCAATTGTCCAATCTGTGGGGCAGGGTTTactcagaaaggtaacctcctccgccacattaaaCTG | 46F_47N_49P_50I_52G_53A_54G 55F 67L | 2.898396014 |
| 1228 | 4559 | TTCTACTGTCCTGTTTGTGGGGCATACTTTactcagaaaggtaacctcctccgccacattaaaCTG | 46F_47Y_49P_50V_52G_53A_54Y_55F_67L | 2.897942431 |
| 1229 | 6695 | CTCAACTGCCCCATCTGCGGGTTTAGCTGCactcagaaaggtaacctcctccgccacattaaaCTG | 46L 47N 49P 50I 52G 53F 54S 55C 67L | 2.897831647 |
| 1230 | 965 | TTCTTTTGTGACATATGTGGGGCAGGTTTTactcagaaaggtaacctcctccgccacattaaaCTG | 46F_47F_49D_50I_52G_53A_54G_55F 67L | 2.89737216 |
| 1231 | 4427 | TTCTATTGTGAAGTTTGTGGGGCAATCTTTactcagaaaggtaacctcctccgccacattaaaCTG | 46F_47Y_49E_50V_52G_53A_54I_55F_67L | 2.89413739 |
| 1232 | 4134 | TTCTGGTGTGACGTGTGTGGGGCACGCTTTactcagaaaggtaacctcctccgccacattaaaCTG | 46F_47W_49D_50V_52G_53A_54R_55F_67L | 2.893479913 |
| 1233 | 2940 | TTCCAATGTGAAATCTGTGGGGCAGCATTTactcagaaaggtaacctcctccgccacattaaaCTG | 46F_47Q_49E_50I_52G_53A_54A 55F 67L | 2.890539027 |
| 1234 | 3472 | TTCTCTTGTGAAGTATGTGGGGCACCCTTTactcagaaaggtaacctcctccgccacattaaaCTG | 46F_47S_49E_50V_52G_53A_54P_55F 67L | 2.890099109 |
| 1235 | 4546 | TTCTATTGTCCTGTGTGTGGGGCACACTTTactcagaaaggtaacctcctccgccacattaaaCTG | 46F_47Y_49P_50V_52G_53A_54H 55F 67L | 2.8898658 |
| 1236 | 1386 | TTCGGGTGTCCTATTTGTGGGGCACACTTTactcagaaaggtaacctcctccgccacattaaaCTG | 46F_47G_49P_50I_52G_53A_54H_55F_67L | 2.889780831 |
| 1237 | 7736 | TTGCATTGCGAAATATGTGGCTTCACTTGTactcagaaaggtaacctcctccgccacattaaaCTG | 46L_47H_49E_50I_52G_53F_54T_55C_67L | 2.888962206 |
| 1238 | 4553 | TTCTACTGTCCGGTGTGTGGGGCACAATTTactcagaaaggtaacctcctccgccacattaaaCTG | 46F_47Y_49P_50V_52G_53A_54Q 55F 67L | 2.884894386 |
| 1239 | 3252 | TTCAGGTGTAACATTTGTGGGGCACCGTTTactcagaaaggtaacctcctccgccacattaaaCTG | 46F_47R_49N_50I_52G_53A_54P_55F_67L | 2.884206307 |
| 1240 | 2713 | TTCAACTGTGAGATATGTGGGGCACAATTTactcagaaaggtaacctcctccgccacattaaaCTG | 46F_47N_49E_50I_52G_53A_54Q_55F_67L | 2.883127674 |
| 1241 | 1277 | TTCGGATGTGAAATTTGTGGGGCAGTGTTTactcagaaaggtaacctcctccgccacattaaaCTG | 46F_47G_49E_50I_52G_53A_54V_55F_67L | 2.88219703 |
| 1242 | 6559 | CTGTTTTGTCCAATCTGCGGGTTCTATTGCactcagaaaggtaacctcctccgccacattaaaCTG | 46L_47F_49P_50I_52G_53F_54Y_55C_67L | 2.880876698 |
| 1243 | 1180 | TTCTTCTGTCCCGTGTGTGGGGCAGCATTTactcagaaaggtaacctcctccgccacattaaaCTG | 46F_47F_49P_50V_52G_53A_54A_55F_67L | 2.879974098 |
| 1244 | 240 | TTCTGCTGTGACATTTGTGGGGCAGCATTTactcagaaaggtaacctcctccgccacattaaaCTG | 46F_47C_49D_50I_52G_53A_54A_55F_67L | 2.879177741 |
| 1245 | 4212 | TTCTGGTGTAATATCTGTGGGGCACCATTTactcagaaaggtaacctcctccgccacattaaaCTG | 46F_47W_49N_50I_52G_53A_54P_55F 67L | 2.87892669 |
| 1246 | 672 | TTCGACTGTCCCATCTGTGGGGCACCGTTTactcagaaaggtaacctcctccgccacattaaaCTG | 46F_47D_49P_50I_52G_53A_54P 55F 67L | 2.877803347 |
| 1247 | 7957 | CTGTGGTGCGAGATTTGTGGATTTGTATGCactcagaaaggtaacctcctccgccacattaaaCTG | 46L 47W_49E_50I_52G_53F_54V_55C_67L | 2.877631856 |
| 1248 | 4093 | TTCTGGTGTGACATCTGTGGGGCACAGTTTactcagaaaggtaacctcctccgccacattaaaCTG | 46F_47W_49D_50I_52G_53A_54Q_55F_67L | 2.876777335 |
| 1249 | 1300 | TTCGGGTGTGAGGTCTGTGGGGCAGCTTTTactcagaaaggtaacctcctccgccacattaaaCTG | 46F_47G_49E_50V_52G_53A_54A_55F_67L | 2.87647245 |
| 1250 | 2865 | TTCAACTGTCCAGTGTGTGGGGCAGGGTTTactcagaaaggtaacctcctccgccacattaaaCTG | 46F_47N_49P_50V_52G_53A_54G_55F_67L | 2.87589373 |
| 1251 | 4517 | TTCTACTGTCCTATTTGTGGGGCAGTGTTTactcagaaaggtaacctcctccgccacattaaaCTG | 46F_47Y_49P_50I_52G_53A_54V_55F_67L | 2.875230307 |
| 1252 | 6817 | CTCTGGTGCCCCATCTGCGGATTCGTGTGCactcagaaaggtaacctcctccgccacattaaaCTG | 46L 47W_49P_50I_52G_53F_54V_55C_67L | 2.870823367 |
| 1253 | 6193 | TTCGGCTGCCCCATTTGCGGCAGGCAGTTTactcagaaaggtaacctcctccgccacattaaaCTG | 46F_47G_49P_50I_52G_53R_54Q_55F_67L | 2.869553248 |
| 1254 | 1317 | TTCGGATGTGAAGTCTGTGGGGCAGTGTTTactcagaaaggtaacctcctccgccacattaaaCTG | 46F_47G_49E_50V_52G_53A_54V_55F_67L | 2.869012109 |
| 1255 | 1159 | TTCTTTTGTCCGATTTGTGGGGCATACTTTactcagaaaggtaacctcctccgccacattaaaCTG | 46F_47F_49P_50I_52G_53A_54Y_55F_67L | 2.868305897 |
| 1256 | 1077 | TTCTTCTGTGAGGTGTGTGGGGCAGTGTTTactcagaaaggtaacctcctccgccacattaaaCTG | 46F_47F_49E_50V_52G_53A_54V_55F_67L | 2.867820835 |
| 1257 | 4500 | TTCTACTGTCCAATCTGTGGGGCAGCATTTactcagaaaggtaacctcctccgccacattaaaCTG | 46F_47Y_49P_50I_52G_53A_54A_55F_67L | 2.860566877 |
| 1258 | 780 | TTCGAGTGTGAGATCTGTGGGGCAGCGTTTactcagaaaggtaacctcctccgccacattaaaCTG | 46F_47E_49E_50I_52G_53A_54A_55F_67L | 2.860515261 |
| 1259 | 3433 | TTCTCGTGTGAAATCTGTGGGGCACAGTTTactcagaaaggtaacctcctccgccacattaaaCTG | 46F_47S_49E_50I_52G_53A 54Q_55F_67L | 2.859770788 |
| 1260 | 1183 | TTCTTCTGTCCGGTGTGTGGGGCAGAATTTactcagaaaggtaacctcctccgccacattaaaCTG | 46F_47F_49P_50V_52G_53A_54E_55F_67L | 2.856823723 |
| 1261 | 6810 | CTGTGGTGCCCCATTTGTGGCTTCATGTGTactcagaaaggtaacctcctccgccacattaaaCTG | 46L_47W_49P_50I_52G_53F_54M_55C_67L | 2.855738916 |
| 1262 | 4441 | TTCTATTGTAACATATGTGGGGCATGTTTTactcagaaaggtaacctcctccgccacattaaaCTG | 46F_47Y_49N_50I_52G_53A_54C_55F_67L | 2.855172985 |
| 1263 | 1426 | TTCGGCTGTCCAGTATGTGGGGCACACTTTactcagaaaggtaacctcctccgccacattaaaCTG | 46F_47G_49P_50V_52G_53A_54H_55F_67L | 2.853598177 |
| 1264 | 199 | TTCGCGTGTCCCATTTGTGGGGCATATTTTactcagaaaggtaacctcctccgccacattaaaCTG | 46F_47A_49P_50I_52G_53A_54Y_55F_67L | 2.851306116 |
| 1265 | 4199 | TTCTGGTGTGAGGTCTGTGGGGCATACTTTactcagaaaggtaacctcctccgccacattaaaCTG | 46F_47W_49E_50V_52G_53A_54Y_55F_67L | 2.850862019 |
| 1266 | 1 | TTCGCCTGTGACATCTGTGGGGCATGCTTTactcagaaaggtaacctcctccgccacattaaaCTG | 46F_47A_49D_50I_52G_53A_54C_55F_67L | 2.847596651 |
| 1267 | 1665 | TTCCATTGTCCGGTGTGTGGGGCAGGCTTTactcagaaaggtaacctcctccgccacattaaaCTG | 46F_47H_49P_50V_52G_53A_54G_55F_67L | 2.846575266 |
| 1268 | 6824 | CTTTACTGTCCCATATGCGGGTTTTTCTGTactcagaaaggtaacctcctccgccacattaaaCTG | 46L_47Y 49P 50I 52G 53F 54F 55C 67L | 2.845124896 |
| 1269 | 2835 | TTCAACTGTCCCATCTGTGGGGCATCTTTTactcagaaaggtaacctcctccgccacattaaaCTG | 46F_47N_49P_50I_52G_53A_54S_55F_67L | 2.844011038 |
| 1270 | 977 | TTCTTTTGTGACATCTGTGGGGCAGTGTTTactcagaaaggtaacctcctccgccacattaaaCTG | 46F_47F_49D_50I_52G_53A_54V_55F_67L | 2.842053855 |
| 1271 | 1431 | TTCGGATGTCCAGTATGTGGGGCAAACTTTactcagaaaggtaacctcctccgccacattaaaCTG | 46F_47G_49P_50V_52G_53A_54N_55F_67L | 2.840268415 |
| 1272 | 3541 | TTCTCATGTCCTATATGTGGGGCATGTTTTactcagaaaggtaacctcctccgccacattaaaCTG | 46F_47S_49P_50I_52G_53A_54C_55F_67L | 2.840132311 |
| 1273 | 40 | TTCGCCTGTGATGTCTGTGGGGCAGCTTTTactcagaaaggtaacctcctccgccacattaaaCTG | 46F_47A_49D_50V_52G_53A_54A_55F_67L | 2.838525742 |
| 1274 | 4509 | TTCTATTGTCCAATCTGTGGGGCATTGTTTactcagaaaggtaacctcctccgccacattaaaCTG | 46F_47Y_49P_50I_52G_53A_54L_55F_67L | 2.833828918 |
| 1275 | 1071 | TTCTTCTGTGAGGTCTGTGGGGCAAACTTTactcagaaaggtaacctcctccgccacattaaaCTG | 46F_47F_49E_50V_52G_53A_54N_55F_67L | 2.831824761 |
| 1276 | 4197 | TTCTGGTGTGAGGTGTGTGGGGCAGTCTTTactcagaaaggtaacctcctccgccacattaaaCTG | 46F_47W_49E_50V_52G_53A_54V_55F 67L | 2.831581409 |
| 1277 | 1752 | TTCATCTGTGAGATTTGTGGGGCACCTTTTactcagaaaggtaacctcctccgccacattaaaCTG | 46F_47I_49E_50I_52G_53A_54P 55F_67L | 2.830924682 |
| 1278 | 3436 | TTCTCCTGTGAAATTTGTGGGGCAACTTTTactcagaaaggtaacctcctccgccacattaaaCTG | 46F_47S_49E_50I_52G_53A_54T_55F_67L | 2.830111648 |
| 1279 | 6495 | CTCTGCTGCCCCATCTGTGGTTTCAGTTGCactcagaaaggtaacctcctccgccacattaaaCTG | 46L_47C_49P_50I_52G_53F_54S_55C_67L | 2.829488172 |
| 1280 | 4543 | TTCTATTGTCCCGTGTGTGGGGCAGAGTTTactcagaaaggtaacctcctccgccacattaaaCTG | 46F_47Y_49P_50V_52G_53A_54E_55F_67L | 2.82920043 |
| 1281 | 230 | TTCGCCTGTCCTGTCTGTGGGGCAATGTTTactcagaaaggtaacctcctccgccacattaaaCTG | 46F_47A_49P_50V_52G_53A_54M_55F_67L | 2.828926863 |
| 1282 | 3545 | TTCTCTTGTCCGATTTGTGGGGCAGGGTTTactcagaaaggtaacctcctccgccacattaaaCTG | 46F_47S_49P_50I_52G_53A 54G_55F 67L | 2.828392434 |
| 1283 | 6196 | TTTGGTTGCCCAATCTGCGGGAGGACATTTactcagaaaggtaacctcctccgccacattaaaCTG | 46F_47G_49P_50I_52G_53R_54T_55F_67L | 2.827751524 |
| 1284 | 1013 | TTCTTTTGTGACGTTTGTGGGGCACAATTTactcagaaaggtaacctcctccgccacattaaaCTG | 46F_47F_49D_50V_52G_53A_54Q_55F_67L | 2.823885742 |
| 1285 | 6585 | CTACATTGTCCTATCTGCGGCTTTGGGTGTactcagaaaggtaacctcctccgccacattaaaCTG | 46L_47H_49P_50I_52G_53F_54G_55C_67L | 2.82330936 |
| 1286 | 4551 | TTCTACTGTCCAGTCTGTGGGGCAAATTTTactcagaaaggtaacctcctccgccacattaaaCTG | 46F_47Y_49P_50V_52G_53A_54N_55F_67L | 2.821262016 |
| 1287 | 7894 | TTAAGCTGCGAGATTTGTGGCTTTAGATGCactcagaaaggtaacctcctccgccacattaaaCTG | 46L_47S_49E_50I_52G_53F_54R_55C_67L | 2.82113688 |
| 1288 | 54 | TTCGCTTGTGATGTATGTGGGGCACGGTTTactcagaaaggtaacctcctccgccacattaaaCTG | 46F_47A_49D_50V_52G_53A_54R_55F_67L | 2.8204591 |
| 1289 | 4556 | TTCTATTGTCCGGTGTGTGGGGCAACTTTTactcagaaaggtaacctcctccgccacattaaaCTG | 46F_47Y_49P_50V_52G_53A 54T 55F 67L | 2.814076 |
| 1290 | 595 | TTCGATTGTGAAGTCTGTGGGGCATCTTTTactcagaaaggtaacctcctccgccacattaaaCTG | 46F_47D_49E_50V_52G_53A_54S_55F_67L | 2.812903931 |
| 1291 | 1156 | TTCTTTTGTCCCATATGTGGGGCAACGTTTactcagaaaggtaacctcctccgccacattaaaCTG | 46F_47F_49P_50I_52G_53A_54T_55F_67L | 2.812670463 |
| 1292 | 2945 | TTCCAGTGTGAAATCTGTGGGGCAGGCTTTactcagaaaggtaacctcctccgccacattaaaCTG | 46F_47Q_49E_50I_52G_53A_54G_55F_67L | 2.810773904 |
| 1293 | 4091 | TTCTGGTGTGACATTTGTGGGGCAAACTTTactcagaaaggtaacctcctccgccacattaaaCTG | 46F_47W_49D_50I_52G_53A_54N_55F_67L | 2.808554552 |
| 1294 | 6548 | CTGTTTTGTCCCATCTGTGGCTTCAAGTGTactcagaaaggtaacctcctccgccacattaaaCTG | 46L 47F_49P_50I_52G_53F_54K_55C_67L | 2.80849036 |
| 1295 | 2465 | TTCATGTGTGAGATCTGTGGGGCAGGTTTTactcagaaaggtaacctcctccgccacattaaaCTG | 46F_47M_49E_50I_52G_53A_54G_55F_67L | 2.807985113 |
| 1296 | 6740 | CTTAGCTGCCCCATCTGCGGTTTCGCCTGCactcagaaaggtaacctcctccgccacattaaaCTG | 46L_47S_49P_50I_52G_53F_54A_55C_67L | 2.806287092 |
| 1297 | 1151 | TTCTTCTGTCCCATTTGTGGGGCAAATTTTactcagaaaggtaacctcctccgccacattaaaCTG | 46F_47F_49P_50I_52G_53A_54N_55F 67L | 2.805205861 |
| 1298 | 190 | TTCGCCTGTCCTATCTGTGGGGCAATGTTTactcagaaaggtaacctcctccgccacattaaaCTG | 46F_47A_49P_50I_52G_53A_54M_55F_67L | 2.801637805 |
| 1299 | 6543 | CTGTTCTGCCCCATCTGCGGATTCGAGTGTactcagaaaggtaacctcctccgccacattaaaCTG | 46L_47F_49P_50I_52G_53F_54E_55C_67L | 2.80163717 |
| 1300 | 3700 | TTCACATGTGAAGTGTGTGGGGCAGCTTTTactcagaaaggtaacctcctccgccacattaaaCTG | 46F_47T_49E_50V_52G_53A_54A_55F_67L | 2.800951092 |
| 1301 | 1066 | TTCTTCTGTGAGGTTTGTGGGGCACACTTTactcagaaaggtaacctcctccgccacattaaaCTG | 46F 47F_49E_50V_52G_53A_54H 55F 67L | 2.798298076 |
| 1302 | 1132 | TTCTTCTGTAACGTGTGTGGGGCACCCTTTactcagaaaggtaacctcctccgccacattaaaCTG | 46F_47F_49N_50V_52G_53A_54P_55F_67L | 2.798116435 |
| 1303 | 7687 | CTCTTCTGCGAAATCTGCGGGTTCATATGTactcagaaaggtaacctcctccgccacattaaaCTG | 46L 47F_49E_50I_52G_53F_54I_55C_67L | 2.791588272 |
| 1304 | 6754 | CTAAGCTGCCCAATATGCGGGTTCCGGTGTactcagaaaggtaacctcctccgccacattaaaCTG | 46L_47S_49P_50I_52G_53F_54R_55C_67L | 2.790475724 |
| 1305 | 1188 | TTCTTCTGTCCCGTGTGTGGGGCAAAGTTTactcagaaaggtaacctcctccgccacattaaaCTG | 46F 47F 49P 50V_52G_53A_54K 55F 67L | 2.78732375 |
| 1306 | 1391 | TTCGGGTGTCCAATCTGTGGGGCAAACTTTactcagaaaggtaacctcctccgccacattaaaCTG | 46F_47G_49P_50I_52G_53A_54N_55F_67L | 2.786621359 |
| 1307 | 4331 | TTCTATTGTGATATTTGTGGGGCAAACTTTactcagaaaggtaacctcctccgccacattaaaCTG | 46F_47Y_49D_50I_52G_53A_54N_55F_67L | 2.784164895 |
| 1308 | 4090 | TTCTGGTGTGACATTTGTGGGGCAATGTTTactcagaaaggtaacctcctccgccacattaaaCTG | 46F_47W_49D_50I_52G_53A_54M_55F_67L | 2.783984086 |
| 1309 | 301 | TTCTGCTGTGAAATCTGTGGGGCATGTTTTactcagaaaggtaacctcctccgccacattaaaCTG | 46F 47C_49E_50I_52G_53A_54C_55F 67L | 2.782113949 |
| 1310 | 2875 | TTCAATTGTCCCGTATGTGGGGCAAGTTTTactcagaaaggtaacctcctccgccacattaaaCTG | 46F_47N_49P_50V_52G_53A_54S_55F_67L | 2.78200685 |
| 1311 | 4274 | TTCTGGTGTCCTATTTGTGGGGCACGATTTactcagaaaggtaacctcctccgccacattaaaCTG | 46F_47W_49P_50I_52G_53A_54R_55F_67L | 2.781296391 |
| 1312 | 3185 | TTCCGTTGTGAGATCTGTGGGGCAGGTTTTactcagaaaggtaacctcctccgccacattaaaCTG | 46F_47R_49E_50I_52G_53A_54G_55F_67L | 2.780237343 |
| 1313 | 3594 | TTCTCTTGTCCAGTATGTGGGGCACGATTTactcagaaaggtaacctcctccgccacattaaaCTG | 46F 47S_49P_50V_52G_53A_54R_55F_67L | 2.780203697 |
| 1314 | 6819 | CTGTGGTGTCCCATCTGCGGCTTTTATTGCactcagaaaggtaacctcctccgccacattaaaCTG | 46L_47W_49P_50I_52G_53F_54Y_55C 67L | 2.779350775 |
| 1315 | 6823 | TTGTATTGTCCGATCTGCGGGTTCGAGTGCactcagaaaggtaacctcctccgccacattaaaCTG | 46L 47Y_49P_50I_52G_53F_54E_55C_67L | 2.778418184 |
| 1316 | 1157 | TTCTTCTGTCCCATCTGTGGGGCAGTCTTTactcagaaaggtaacctcctccgccacattaaaCTG | 46F_47F_49P_50I_52G_53A_54V_55F_67L | 2.778290562 |
| 1317 | 4121 | TTCTGGTGTGATGTATGTGGGGCATGCTTTactcagaaaggtaacctcctccgccacattaaaCTG | 46F_47W_49D_50V_52G_53A_54C_55F_67L | 2.776846872 |
| 1318 | 3556 | TTCTCATGTCCCATCTGTGGGGCAACTTTTactcagaaaggtaacctcctccgccacattaaaCTG | 46F_47S_49P_50I_52G_53A_54T 55F 67L | 2.776174605 |
| 1319 | 120 | TTCGCCTGTAACATTTGTGGGGCAGCATTTactcagaaaggtaacctcctccgccacattaaaCTG | 46F_47A_49N_50I_52G_53A_54A_55F_67L | 2.773478162 |
| 1320 | 3473 | TTCTCCTGTGAGGTGTGTGGGGCACAGTTTactcagaaaggtaacctcctccgccacattaaaCTG | 46F_47S_49E_50V_52G_53A_54Q_55F_67L | 2.771582535 |
| 1321 | 1635 | TTCCACTGTCCTATCTGTGGGGCATCCTTTactcagaaaggtaacctcctccgccacattaaaCTG | 46F_47H_49P_50I_52G_53A_54S_55F_67L | 2.769277633 |
| 1322 | 187 | TTCGCGTGTCCTATTTGTGGGGCAATCTTTactcagaaaggtaacctcctccgccacattaaaCTG | 46F_47A_49P_50I_52G_53A_54I_55F_67L | 2.764805282 |
| 1323 | 4424 | TTCTATTGTGAGGTCTGTGGGGCATTCTTTactcagaaaggtaacctcctccgccacattaaaCTG | 46F_47Y_49E_50V_52G_53A_54F_55F_67L | 2.760123873 |
| 1324 | 1008 | TTCTTCTGTGATGTGTGTGGGGCAAAATTTactcagaaaggtaacctcctccgccacattaaaCTG | 46F_47F_49D_50V_52G_53A_54K_55F_67L | 2.755211237 |
| 1325 | 182 | TTCGCATGTCCAATCTGTGGGGCAGACTTTactcagaaaggtaacctcctccgccacattaaaCTG | 46F_47A_49P_50I_52G_53A_54D_55F_67L | 2.754946511 |
| 1326 | 6580 | CTGCACTGCCCAATCTGTGGATTCGCGTGCactcagaaaggtaacctcctccgccacattaaaCTG | 46L_47H_49P_50I_52G_53F_54A_55C_67L | 2.752626604 |
| 1327 | 2512 | TTCATGTGTGAAGTCTGTGGGGCACCCTTTactcagaaaggtaacctcctccgccacattaaaCTG | 46F_47M_49E_50V_52G_53A 54P 55F 67L | 2.752137451 |
| 1328 | 4196 | TTCTGGTGTGAAGTTTGTGGGGCAACATTTactcagaaaggtaacctcctccgccacattaaaCTG | 46F_47W_49E_50V_52G_53A 54T 55F 67L | 2.751896795 |
| 1329 | 4507 | TTCTATTGTCCTATATGTGGGGCAATTTTTactcagaaaggtaacctcctccgccacattaaaCTG | 46F_47Y_49P_50I 52G_53A_54I 55F_67L | 2.750595708 |
| 1330 | 6596 | CTGCACTGCCCTATCTGTGGCTTCACATGTactcagaaaggtaacctcctccgccacattaaaCTG | 46L_47H_49P_50I_52G_53F_54T_55C_67L | 2.749652356 |
| 1331 | 7895 | CTGAGTTGCGAAATCTGCGGCTTCAGCTGTactcagaaaggtaacctcctccgccacattaaaCTG | 46L 47S_49E_50I_52G_53F_54S_55C_67L | 2.745721271 |
| 1332 | 6725 | TTGCGCTGTCCCATATGCGGCTTCGGCTGCactcagaaaggtaacctcctccgccacattaaaCTG | 46L_47R_49P_50I_52G_53F_54G_55C_67L | 2.744161798 |
| 1333 | 4096 | TTCTGGTGTGATATCTGTGGGGCAACATTTactcagaaaggtaacctcctccgccacattaaaCTG | 46F_47W_49D_50I_52G_53A_54T_55F_67L | 2.743305046 |
| 1334 | 7686 | CTGTTTTGCGAAATCTGCGGATTTCATTGTactcagaaaggtaacctcctccgccacattaaaCTG | 46L_47F_49E_50I_52G_53F_54H_55C_67L | 2.742445049 |
| 1335 | 4308 | TTCTGGTGTCCAGTGTGTGGGGCAAAGTTTactcagaaaggtaacctcctccgccacattaaaCTG | 46F_47W_49P_50V_52G_53A_54K_55F_67L | 2.741880848 |
| 1336 | 6452 | TTCTACTGCCCAATTTGCGGCCGGCCATTTactcagaaaggtaacctcctccgccacattaaaCTG | 46F_47Y_49P_50I_52G_53R_54P_55F_67L | 2.739320704 |
| 1337 | 6805 | CTGTGGTGTCCAATCTGCGGTTTTGGCTGCactcagaaaggtaacctcctccgccacattaaaCTG | 46L_47W_49P_50I_52G_53F_54G_55C_67L | 2.738602589 |
| 1338 | 4445 | TTCTATTGTAACATTTGTGGGGCAGGCTTTactcagaaaggtaacctcctccgccacattaaaCTG | 46F_47Y_49N_50I_52G_53A_54G_55F_67L | 2.736208045 |
| 1339 | 6806 | CTGTGGTGTCCTATCTGCGGGTTCCACTGTactcagaaaggtaacctcctccgccacattaaaCTG | 46L_47W_49P_50I_52G_53F_54H_55C_67L | 2.735147942 |
| 1340 | 715 | TTCGACTGTCCCGTCTGTGGGGCATCCTTTactcagaaaggtaacctcctccgccacattaaaCTG | 46F_47D_49P_50V_52G_53A_54S_55F_67L | 2.734403808 |
| 1341 | 16 | TTCGCCTGTGACATTTGTGGGGCAACTTTTactcagaaaggtaacctcctccgccacattaaaCTG | 46F_47A_49D_50I_52G_53A_54T_55F_67L | 2.731196501 |
| 1342 | 3476 | TTCAGCTGTGAGGTTTGTGGGGCAACTTTTactcagaaaggtaacctcctccgccacattaaaCTG | 46F_47S_49E_50V_52G_53A_54T_55F_67L | 2.730587103 |
| 1343 | 7946 | CTGTGGTGCGAAATTTGCGGGTTTCATTGCactcagaaaggtaacctcctccgccacattaaaCTG | 46L_47W_49E_50I_52G_53F_54H 55C_67L | 2.729168063 |
| 1344 | 3437 | TTCTCTTGTGAAATTTGTGGGGCAGTCTTTactcagaaaggtaacctcctccgccacattaaaCTG | 46F_47S_49E_50I_52G_53A_54V_55F_67L | 2.72881873 |
| 1345 | 1144 | TTCTTCTGTCCTATTTGTGGGGCATTTTTTactcagaaaggtaacctcctccgccacattaaaCTG | 46F_47F_49P_50I_52G_53A_54F_55F_67L | 2.726568403 |
| 1346 | 3585 | TTCAGCTGTCCCGTCTGTGGGGCAGGATTTactcagaaaggtaacctcctccgccacattaaaCTG | 46F_47S_49P_50V_52G_53A_54G_55F_67L | 2.72640891 |
| 1347 | 7965 | CTATACTGTGAAATCTGCGGATTCGGGTGCactcagaaaggtaacctcctccgccacattaaaCTG | 46L 47Y_49E_50I_52G_53F_54G_55C_67L | 2.725553342 |
| 1348 | 2980 | TTCCAATGTGAAGTTTGTGGGGCAGCCTTTactcagaaaggtaacctcctccgccacattaaaCTG | 46F_47Q_49E_50V_52G_53A_54A_55F_67L | 2.724227663 |
| 1349 | 239 | TTCGCATGTCCCGTTTGTGGGGCATATTTTactcagaaaggtaacctcctccgccacattaaaCTG | 46F_47A_49P_50V_52G_53A_54Y_55F_67L | 2.724222007 |
| 1350 | 6566 | CTGGGCTGCCCAATTTGCGGCTTTCACTGCactcagaaaggtaacctcctccgccacattaaaCTG | 46L 47G_49P_50I_52G_53F_54H_55C_67L | 2.723597332 |
| 1351 | 3596 | TTCTCTTGTCCTGTTTGTGGGGCAACATTTactcagaaaggtaacctcctccgccacattaaaCTG | 46F_47S_49P_50V_52G_53A_54T_55F_67L | 2.722817935 |
| 1352 | 1076 | TTCTTCTGTGAGGTGTGTGGGGCAACATTTactcagaaaggtaacctcctccgccacattaaaCTG | 46F_47F_49E_50V_52G_53A_54T_55F_67L | 2.719418187 |
| 1353 | 1514 | TTCCATTGTGAAATATGTGGGGCACGATTTactcagaaaggtaacctcctccgccacattaaaCTG | 46F_47H_49E_50I_52G_53A_54R_55F_67L | 2.718620428 |
| 1354 | 4279 | TTCTGGTGTCCTATTTGTGGGGCATATTTTactcagaaaggtaacctcctccgccacattaaaCTG | 46F_47W_49P_50I_52G_53A_54Y_55F_67L | 2.71850382 |
| 1355 | 2701 | TTCAACTGTGAGATTTGTGGGGCATGTTTTactcagaaaggtaacctcctccgccacattaaaCTG | 46F_47N_49E_50I_52G_53A_54C_55F_67L | 2.718411543 |
| 1356 | 3180 | TTCCGATGTGAGATATGTGGGGCAGCCTTTactcagaaaggtaacctcctccgccacattaaaCTG | 46F_47R_49E_50I_52G_53A_54A_55F_67L | 2.717390692 |
| 1357 | 7734 | CTCCACTGTGAGATTTGTGGGTTCCGATGCactcagaaaggtaacctcctccgccacattaaaCTG | 46L_47H_49E_50I_52G_53F_54R_55C_67L | 2.714387651 |
| 1358 | 107 | TTCGCATGTGAAGTCTGTGGGGCAATCTTTactcagaaaggtaacctcctccgccacattaaaCTG | 46F_47A_49E_50V_52G_53A_54I 55F 67L | 2.712284845 |
| 1359 | 2412 | TTCATGTGTGACATTTGTGGGGCACCCTTTactcagaaaggtaacctcctccgccacattaaaCTG | 46F_47M_49D_50I_52G_53A_54P_55F_67L | 2.709134586 |
| 1360 | 1621 | TTCCACTGTCCTATTTGTGGGGCATGCTTTactcagaaaggtaacctcctccgccacattaaaCTG | 46F_47H_49P_50I_52G_53A_54C_55F_67L | 2.708131057 |
| 1361 | 6550 | CTTTTCTGCCCAATCTGCGGATTCATGTGTactcagaaaggtaacctcctccgccacattaaaCTG | 46L 47F_49P_50I_52G_53F_54M_55C_67L | 2.707743868 |
| 1362 | 69 | TTCGCATGTGAGATATGTGGGGCACTGTTTactcagaaaggtaacctcctccgccacattaaaCTG | 46F_47A_49E_50I_52G_53A 54L 55F 67L | 2.706091143 |
| 1363 | 963 | TTCTTTTGTGACATATGTGGGGCAGAGTTTactcagaaaggtaacctcctccgccacattaaaCTG | 46F_47F_49D_50I_52G_53A_54E_55F_67L | 2.704063421 |
| 1364 | 2585 | TTCATGTGTCCAATTTGTGGGGCAGGATTTactcagaaaggtaacctcctccgccacattaaaCTG | 46F_47M_49P_50I_52G_53A_54G_55F_67L | 2.702867692 |
| 1365 | 2752 | TTCAATTGTGAGGTTTGTGGGGCACCCTTTactcagaaaggtaacctcctccgccacattaaaCTG | 46F_47N_49E_50V_52G_53A_54P 55F 67L | 2.702380534 |
| 1366 | 252 | TTCTGTTGTGATATCTGTGGGGCACCTTTTactcagaaaggtaacctcctccgccacattaaaCTG | 46F_47C_49D_50I_52G_53A_54P 55F_67L | 2.7019869 |
| 1367 | 792 | TTCGAATGTGAAATCTGTGGGGCACCCTTTactcagaaaggtaacctcctccgccacattaaaCTG | 46F_47E_49E_50I_52G_53A_54P 55F_67L | 2.701740149 |
| 1368 | 6581 | CTGCACTGCCCAATTTGCGGGTTCTGCTGTactcagaaaggtaacctcctccgccacattaaaCTG | 46L_47H_49P_50I_52G_53F_54C_55C_67L | 2.700554876 |
| 1369 | 4310 | TTCTGGTGTCCAGTCTGTGGGGCAATGTTTactcagaaaggtaacctcctccgccacattaaaCTG | 46F_47W_49P_50V_52G_53A_54M_55F_67L | 2.696027176 |
| 1370 | 1186 | TTCTTTTGTCCTGTCTGTGGGGCACATTTTactcagaaaggtaacctcctccgccacattaaaCTG | 46F_47F_49P_50V_52G_53A 54H 55F 67L | 2.694707642 |
| 1371 | 1985 | TTCAAATGTGAAATATGTGGGGCAGGTTTTactcagaaaggtaacctcctccgccacattaaaCTG | 46F_47K_49E_50I_52G_53A_54G_55F_67L | 2.694443429 |
| 1372 | 4135 | TTCTGGTGTGACGTCTGTGGGGCATCTTTTactcagaaaggtaacctcctccgccacattaaaCTG | 46F_47W_49D_50V_52G_53A_54S_55F_67L | 2.692306721 |
| 1373 | 4544 | TTCTACTGTCCCGTCTGTGGGGCATTCTTTactcagaaaggtaacctcctccgccacattaaaCTG | 46F_47Y_49P_50V_52G_53A_54F_55F_67L | 2.692182523 |
| 1374 | 4336 | TTCTACTGTGACATCTGTGGGGCAACTTTTactcagaaaggtaacctcctccgccacattaaaCTG | 46F_47Y_49D_50I_52G_53A_54T_55F_67L | 2.691637205 |
| 1375 | 473 | TTCTGCTGTCCCGTTTGTGGGGCACAATTTactcagaaaggtaacctcctccgccacattaaaCTG | 46F_47C_49P_50V_52G_53A_54Q_55F_67L | 2.687531745 |
| 1376 | 1199 | TTCTTTTGTCCCGTGTGTGGGGCATATTTTactcagaaaggtaacctcctccgccacattaaaCTG | 46F_47F_49P_50V_52G_53A_54Y_55F_67L | 2.685576574 |
| 1377 | 4368 | TTCTATTGTGACGTATGTGGGGCAAAATTTactcagaaaggtaacctcctccgccacattaaaCTG | 46F 47Y 49D_50V 52G 53A 54K 55F 67L | 2.684934864 |
| 1378 | 6759 | CTGTCTTGTCCTATCTGTGGTTTCTATTGCactcagaaaggtaacctcctccgccacattaaaCTG | 46L_47S_49P_50I_52G_53F_54Y 55C_67L | 2.681746923 |
| 1379 | 2860 | TTCAACTGTCCCGTGTGTGGGGCAGCCTTTactcagaaaggtaacctcctccgccacattaaaCTG | 46F_47N_49P_50V_52G_53A_54A_55F_67L | 2.681512385 |
| 1380 | 2652 | TTCAATTGTGACATCTGTGGGGCACCTTTTactcagaaaggtaacctcctccgccacattaaaCTG | 46F_47N_49D_50I_52G_53A_54P_55F_67L | 2.681279214 |
| 1381 | 3792 | TTCACATGTCCCATCTGTGGGGCACCGTTTactcagaaaggtaacctcctccgccacattaaaCTG | 46F_47T_49P_50I_52G_53A_54P_55F_67L | 2.678566547 |
| 1382 | 4133 | TTCTGGTGTGACGTTTGTGGGGCACAGTTTactcagaaaggtaacctcctccgccacattaaaCTG | 46F_47W_49D_50V_52G_53A_54Q_55F_67L | 2.675332452 |
| 1383 | 48 | TTCGCCTGTGACGTATGTGGGGCAAAGTTTactcagaaaggtaacctcctccgccacattaaaCTG | 46F_47A_49D_50V_52G_53A_54K 55F 67L | 2.673465759 |
| 1384 | 4314 | TTCTGGTGTCCTGTGTGTGGGGCAAGATTTactcagaaaggtaacctcctccgccacattaaaCTG | 46F_47W_49P_50V_52G_53A 54R 55F 67L | 2.670611051 |
| 1385 | 6715 | TTACAGTGTCCAATCTGCGGATTTAGCTGTactcagaaaggtaacctcctccgccacattaaaCTG | 46L_47Q_49P_50I_52G_53F_54S_55C_67L | 2.667379238 |
| 1386 | 1079 | TTCTTCTGTGAGGTGTGTGGGGCATACTTTactcagaaaggtaacctcctccgccacattaaaCTG | 46F 47F 49E 50V 52G 53A 54Y 55F 67L | 2.664868919 |
| 1387 | 1146 | TTCTTCTGTCCTATCTGTGGGGCACACTTTactcagaaaggtaacctcctccgccacattaaaCTG | 46F_47F_49P_50I_52G_53A_54H_55F_67L | 2.664026555 |
| 1388 | 4266 | TTCTGGTGTCCGATCTGTGGGGCACATTTTactcagaaaggtaacctcctccgccacattaaaCTG | 46F_47W_49P_50I_52G_53A 54H 55F 67L | 2.663987556 |
| 1389 | 3225 | TTCAGATGTGAGGTCTGTGGGGCAGGCTTTactcagaaaggtaacctcctccgccacattaaaCTG | 46F_47R_49E_50V_52G_53A_54G_55F_67L | 2.662989513 |
| 1390 | 4440 | TTCTATTGTAATATCTGTGGGGCAGCCTTTactcagaaaggtaacctcctccgccacattaaaCTG | 46F_47Y_49N_50I_52G_53A_54A_55F_67L | 2.662554997 |
| 1391 | 4557 | TTCTATTGTCCAGTTTGTGGGGCAGTGTTTactcagaaaggtaacctcctccgccacattaaaCTG | 46F_47Y_49P_50V_52G_53A_54V_55F_67L | 2.660726148 |
| 1392 | 6700 | CTCCAGTGCCCGATTTGTGGGTTTGCTTGTactcagaaaggtaacctcctccgccacattaaaCTG | 46L 47Q 49P 50I 52G 53F 54A 55C_67L | 2.660355767 |
| 1393 | 6804 | CTATGGTGTCCAATCTGCGGCTTTTTTTGCactcagaaaggtaacctcctccgccacattaaaCTG | 46L 47W_49P_50I_52G_53F_54F_55C_67L | 2.659219517 |
| 1394 | 4187 | TTCTGGTGTGAAGTCTGTGGGGCAATCTTTactcagaaaggtaacctcctccgccacattaaaCTG | 46F_47W_49E_50V_52G_53A_54I_55F_67L | 2.656484948 |
| 1395 | 4375 | TTCTATTGTGATGTCTGTGGGGCATCCTTTactcagaaaggtaacctcctccgccacattaaaCTG | 46F_47Y_49D_50V_52G_53A_54S_55F_67L | 2.656206477 |
| 1396 | 1513 | TTCCATTGTGAGATCTGTGGGGCACAATTTactcagaaaggtaacctcctccgccacattaaaCTG | 46F_47H_49E_50I_52G_53A_54Q_55F_67L | 2.655602474 |
| 1397 | 4323 | TTCTACTGTGACATTTGTGGGGCAGAGTTTactcagaaaggtaacctcctccgccacattaaaCTG | 46F_47Y_49D_50I_52G_53A_54E 55F 67L | 2.65393074 |
| 1398 | 7610 | TTGGCATGTGAAATCTGCGGTTTTATGTGTactcagaaaggtaacctcctccgccacattaaaCTG | 46L_47A_49E_50I_52G_53F_54M_55C_67L | 2.6531791 |
| 1399 | 2706 | TTCAATTGTGAGATTTGTGGGGCACACTTTactcagaaaggtaacctcctccgccacattaaaCTG | 46F 47N 49E 50I_52G_53A 54H 55F 67L | 2.651918892 |
| 1400 | 3060 | TTCCAATGTCCTATATGTGGGGCAGCTTTTactcagaaaggtaacctcctccgccacattaaaCTG | 46F_47Q_49P_50I_52G_53A_54A 55F 67L | 2.651176447 |
| 1401 | 4510 | TTCTACTGTCCCATTTGTGGGGCAATGTTTactcagaaaggtaacctcctccgccacattaaaCTG | 46F_47Y_49P_50I_52G_53A_54M_55F_67L | 2.64919494 |
| 1402 | 428 | TTCTGTTGTCCAATTTGTGGGGCAAAATTTactcagaaaggtaacctcctccgccacattaaaCTG | 46F_47C_49P_50I_52G_53A_54K_55F_67L | 2.645977926 |
| 1403 | 1269 | TTCGGTTGTGAGATCTGTGGGGCACTTTTTactcagaaaggtaacctcctccgccacattaaaCTG | 46F_47G_49E_50I_52G_53A_54L 55F 67L | 2.645745462 |
| 1404 | 6181 | TTCGGCTGTCCAATCTGTGGCCGTTGTTTCactcagaaaggtaacctcctccgccacattaaaCTG | 46F_47G_49P_50I_52G_53R_54C_55F_67L | 2.645479909 |
| 1405 | 7940 | TTATGGTGTGAGATCTGTGGCTTTGCCTGTactcagaaaggtaacctcctccgccacattaaaCTG | 46L_47W_49E_50I_52G_53F_54A_55C_67L | 2.644172009 |
| 1406 | 2460 | TTCATGTGTGAGATCTGTGGGGCAGCTTTTactcagaaaggtaacctcctccgccacattaaaCTG | 46F_47M_49E_50I_52G_53A_54A_55F_67L | 2.640819743 |
| 1407 | 3581 | TTCTCGTGTCCTGTATGTGGGGCATGCTTTactcagaaaggtaacctcctccgccacattaaaCTG | 46F_47S_49P_50V_52G_53A_54C_55F_67L | 2.639186963 |
| 1408 | 6547 | CTGTTCTGTCCAATATGTGGCTTCATCTGTactcagaaaggtaacctcctccgccacattaaaCTG | 46L_47F_49P_50I_52G_53F_54I_55C_67L | 2.633495742 |
| 1409 | 3554 | TTCAGCTGTCCCATTTGTGGGGCAAGATTTactcagaaaggtaacctcctccgccacattaaaCTG | 46F_47S_49P_50I_52G_53A_54R 55F 67L | 2.630130567 |
| 1410 | 6808 | CTGTGGTGTCCTATCTGTGGGTTTAAGTGTactcagaaaggtaacctcctccgccacattaaaCTG | 46L_47W_49P_50I_52G_53F_54K_55C_67L | 2.630070908 |
| 1411 | 183 | TTCGCCTGTCCAATATGTGGGGCAGAATTTactcagaaaggtaacctcctccgccacattaaaCTG | 46F_47A_49P_50I_52G_53A_54E_55F_67L | 2.627949251 |
| 1412 | 231 | TTCGCTTGTCCAGTCTGTGGGGCAAACTTTactcagaaaggtaacctcctccgccacattaaaCTG | 46F_47A_49P_50V_52G_53A_54N_55F_67L | 2.627659062 |
| 1413 | 671 | TTCGATTGTCCAATCTGTGGGGCAAACTTTactcagaaaggtaacctcctccgccacattaaaCTG | 46F_47D_49P_50I_52G_53A_54N_55F_67L | 2.625736484 |
| 1414 | 540 | TTCGATTGTGAGATCTGTGGGGCAGCATTTactcagaaaggtaacctcctccgccacattaaaCTG | 46F_47D_49E_50I_52G_53A_54A_55F_67L | 2.614234366 |
| 1415 | 64 | TTCGCTTGTGAAATCTGTGGGGCATTCTTTactcagaaaggtaacctcctccgccacattaaaCTG | 46F_47A_49E_50I 52G 53A 54F 55F 67L | 2.60956391 |
| 1416 | 4120 | TTCTGGTGTGACGTCTGTGGGGCAGCATTTactcagaaaggtaacctcctccgccacattaaaCTG | 46F_47W_49D_50V_52G_53A_54A_55F_67L | 2.607829116 |
| 1417 | 3220 | TTCCGATGTGAAGTGTGTGGGGCAGCATTTactcagaaaggtaacctcctccgccacattaaaCTG | 46F_47R_49E_50V_52G_53A_54A_55F_67L | 2.607060188 |
| 1418 | 4508 | TTCTATTGTCCCATCTGTGGGGCAAAGTTTactcagaaaggtaacctcctccgccacattaaaCTG | 46F_47Y_49P_50I_52G_53A_54K_55F_67L | 2.606365493 |
| 1419 | 4208 | TTCTGGTGTAACATCTGTGGGGCAAAGTTTactcagaaaggtaacctcctccgccacattaaaCTG | 46F_47W_49N_50I_52G_53A_54K_55F_67L | 2.606197658 |
| 1420 | 1383 | TTCGGATGTCCGATATGTGGGGCAGAATTTactcagaaaggtaacctcctccgccacattaaaCTG | 46F_47G_49P_50I_52G_53A_54E_55F_67L | 2.602321336 |
| 1421 | 1401 | TTCGGCTGTCCCCAGTGTGGGGCATGTTTTactcagaaaggtaacctcctccgccacattaaaCTG | 46F_47G_49P_50Q_52G_53A_54C_55F_67L | 2.599616135 |
| 1422 | 13 | TTCGCATGTGATATCTGTGGGGCACAGTTTactcagaaaggtaacctcctccgccacattaaaCTG | 46F_47A_49D_50I_52G_53A_54Q_55F_67L | 2.599502069 |
| 1423 | 4215 | TTCTGGTGTAATATATGTGGGGCAAGCTTTactcagaaaggtaacctcctccgccacattaaaCTG | 46F_47W_49N_50I_52G_53A_54S_55F_67L | 2.596861278 |
| 1424 | 1628 | TTCCACTGTCCAATTTGTGGGGCAAAATTTactcagaaaggtaacctcctccgccacattaaaCTG | 46F_47H_49P_50I_52G_53A_54K_55F 67L | 2.59562676 |
| 1425 | 4550 | TTCTATTGTCCAGTGTGTGGGGCAATGTTTactcagaaaggtaacctcctccgccacattaaaCTG | 46F_47Y_49P_50V_52G_53A_54M_55F_67L | 2.590868738 |
| 1426 | 1196 | TTCTTCTGTCCTGTGTGTGGGGCAACTTTTactcagaaaggtaacctcctccgccacattaaaCTG | 46F_47F_49P_50V_52G_53A_54T_55F_67L | 2.587393896 |
| 1427 | 4360 | TTCTACTGTGACGTCTGTGGGGCAGCGTTTactcagaaaggtaacctcctccgccacattaaaCTG | 46F_47Y_49D_50V_52G_53A_54A_55F_67L | 2.587208075 |
| 1428 | 1024 | TTCTTTTGTGAAATTTGTGGGGCATTCTTTactcagaaaggtaacctcctccgccacattaaaCTG | 46F_47F_49E_50I_52G_53A_54F_55F_67L | 2.584119109 |
| 1429 | 1149 | TTCTTTTGTCCTATCTGTGGGGCACTATTTactcagaaaggtaacctcctccgccacattaaaCTG | 46F_47F_49P_50I_52G_53A 54L 55F 67L | 2.58119731 |
| 1430 | 4548 | TTCTACTGTCCCGTCTGTGGGGCAAAATTTactcagaaaggtaacctcctccgccacattaaaCTG | 46F_47Y_49P_50V_52G_53A_54K_55F 67L | 2.580320978 |
| 1431 | 2826 | TTCAACTGTCCGATCTGTGGGGCACATTTTactcagaaaggtaacctcctccgccacattaaaCTG | 46F_47N_49P_50I 52G_53A 54H_55F_67L | 2.57638062 |
| 1432 | 6593 | TTGCACTGCCCTATCTGTGGATTTCAATGCactcagaaaggtaacctcctccgccacattaaaCTG | 46L_47H_49P_50I_52G_53F_54Q_55C_67L | 2.57244456 |
| 1433 | 6463 | CTTGCTTGCCCCATTTGTGGATTTGAGTGCactcagaaaggtaacctcctccgccacattaaaCTG | 46L 47A_49P_50I_52G_53F_54E_55C_67L | 2.568428276 |
| 1434 | 4306 | TTCTGGTGTCCAGTCTGTGGGGCACACTTTactcagaaaggtaacctcctccgccacattaaaCTG | 46F_47W_49P_50V_52G_53A 54H 55F_67L | 2.567398674 |
| 1435 | 6828 | CTGTATTGCCCTATTTGCGGTTTCAAGTGTactcagaaaggtaacctcctccgccacattaaaCTG | 46L_47Y_49P_50I_52G_53F_54K_55C_67L | 2.563841819 |
| 1436 | 2892 | TTCCAGTGTGATATTTGTGGGGCACCCTTTactcagaaaggtaacctcctcogccacattaaaCTG | 46F_47Q_49D_50I_52G_53A_54P_55F_67L | 2.563409697 |
| 1437 | 4365 | TTCTACTGTGATGTCTGTGGGGCAGGTTTTactcagaaaggtaacctcctccgccacattaaaCTG | 46F_47Y_49D_50V_52G_53A_54G_55F_67L | 2.562135928 |
| 1438 | 820 | TTCGAATGTGAAGTGTGTGGGGCAGCATTTactcagaaaggtaacctcctccgccacattaaaCTG | 46F_47E_49E_50V_52G_53A_54A_55F_67L | 2.561073693 |
| 1439 | 6741 | CTGAGCTGTCCCATCTGTGGCTTCTGCTGCactcagaaaggtaacctcctccgccacattaaaCTG | 46L_47S_49P_50I_52G_53F_54C_55C_67L | 2.56040886 |
| 1440 | 6599 | CTTCATTGCCCCATCTGCGGGTTCTACTGCactcagaaaggtaacctcctccgccacattaaaCTG | 46L_47H_49P_50I_52G_53F_54Y_55C_67L | 2.559997241 |
| 1441 | 227 | TTCGCGTGTCCAGTTTGTGGGGCAATATTTactcagaaaggtaacctcctccgccacattaaaCTG | 46F_47A 49P_50V_52G_53A_54I 55F_67L | 2.558663943 |
| 1442 | 1150 | TTCTTCTGTCCCATTTGTGGGGCAATGTTTactcagaaaggtaacctcctccgccacattaaaCTG | 46F_47F_49P_50I_52G_53A_54M 55F 67L | 2.557966029 |
| 1443 | 7635 | CTGTGTTGCGAGATTTGCGGCTTCAGTTGTactcagaaaggtaacctcctccgccacattaaaCTG | 46L_47C_49E_50I_52G_53F_54S_55C_67L | 2.554372769 |
| 1444 | 1069 | TTCTTTTGTGAGGTGTGTGGGGCACTGTTTactcagaaaggtaacctcctccgccacattaaaCTG | 46F_47F_49E_50V_52G_53A_54L_55F_67L | 2.55244661 |
| 1445 | 2225 | TTCCTGTGTGAGATCTGTGGGGCAGGGTTTactcagaaaggtaacctcctccgccacattaaaCTG | 46F_47L 49E 50I 52G 53A 54G 55F 67L | 2.550623653 |
| 1446 | 979 | TTCTTCTGTGACATTTGTGGGGCATATTTTactcagaaaggtaacctcctccgccacattaaaCTG | 46F_47F_49D_50I_52G_53A_54Y_55F_67L | 2.54998241 |
| 1447 | 2112 | TTCAAGTGTCCTATCTGTGGGGCACCTTTTactcagaaaggtaacctcctccgccacattaaaCTG | 46F_47K_49P_50I_52G_53A_54P_55F_67L | 2.549836287 |
| 1448 | 1190 | TTCTTTTGTCCCGTGTGTGGGGCAATGTTTactcagaaaggtaacctcctccgccacattaaaCTG | 46F_47F_49P_50V_52G_53A_54M_55F_67L | 2.546567074 |
| 1449 | 7568 | TTCTGGTGCGAGATCTGTGGCCGCAAGTTTactcagaaaggtaacctcctccgccacattaaaCTG | 46F_47W_49E_50I 52G_53R 54K 55F 67L | 2.546210461 |
| 1450 | 3075 | TTCCAGTGTCCTATCTGTGGGGCAAGCTTTactcagaaaggtaacctcctccgccacattaaaCTG | 46F_47Q_49P_50I_52G_53A_54S_55F 67L | 2.545927307 |
| 1451 | 4085 | TTCTGGTGTGACATATGTGGGGCAGGGTTTactcagaaaggtaacctcctccgccacattaaaCTG | 46F_47W_49D_50I_52G_53A_54G_55F_67L | 2.544431587 |
| 1452 | 3100 | TTCCAGTGTCCAGTTTGTGGGGCAGCCTTTactcagaaaggtaacctcctccgccacattaaaCTG | 46F_47Q_49P_50V_52G_53A_54A 55F 67L | 2.54402036 |
| 1453 | 4270 | TTCTGGTGTCCAATTTGTGGGGCAATGTTTactcagaaaggtaacctcctccgccacattaaaCTG | 46F_47W_49P_50I_52G_53A_54M_55F_67L | 2.543196227 |
| 1454 | 5 | TTCGCCTGTGACATCTGTGGGGCAGGGTTTactcagaaaggtaacctcctccgccacattaaaCTG | 46F_47A_49D_50I_52G_53A_54G_55F_67L | 2.540102555 |
| 1455 | 2505 | TTCATGTGTGAGGTGTGTGGGGCAGGATTTactcagaaaggtaacctcctccgccacattaaaCTG | 46F_47M_49E_50V_52G_53A_54G_55F_67L | 2.539493964 |
| 1456 | 341 | TTCTGCTGTGAAGTATGTGGGGCATGTTTTactcagaaaggtaacctcctccgccacattaaaCTG | 46F_47C_49E_50V_52G_53A_54C_55F_67L | 2.536967863 |
| 1457 | 3374 | TTCTCTTGTGATATCTGTGGGGCACGATTTactcagaaaggtaacctcctccgccacattaaaCTG | 46F_47S_49D_50I_52G_53A_54R_55F_67L | 2.535519303 |
| 1458 | 2708 | TTCAATTGTGAGATTTGTGGGGCAAAATTTactcagaaaggtaacctcctccgccacattaaaCTG | 46F_47N_49E_50I_52G_53A_54K 55F 67L | 2.534600428 |
| 1459 | 4549 | TTCTATTGTCCGGTGTGTGGGGCATTGTTTactcagaaaggtaacctcctccgccacattaaaCTG | 46F_47Y_49P_50V_52G_53A_54L_55F_67L | 2.533066368 |
| 1460 | 7312 | TTCTTTTGCGAGATATGTGGTAGGCCATTCactcagaaaggtaacctcctccgccacattaaaCTG | 46F_47F_49E_50I_52G_53R_54P_55F 67L | 2.53295541 |
| 1461 | 434 | TTCTGCTGTCCCATTTGTGGGGCACGCTTTactcagaaaggtaacctcctccgccacattaaaCTG | 46F_47C_49P_50I_52G_53A_54R_55F_67L | 2.532616642 |
| 1462 | 6184 | TTCGGCTGTCCAATTTGCGGCCGCTTTTTCactcagaaaggtaacctcctccgccacattaaaCTG | 46F_47G_49P_50I_52G_53R_54F_55F_67L | 2.530782578 |
| 1463 | 7720 | TTGCATTGCGAGATTTGTGGATTTGCCTGCactcagaaaggtaacctcctccgccacattaaaCTG | 46L_47H_49E_50I_52G_53F_54A_55C_67L | 2.53012892 |
| 1464 | 1191 | TTCTTCTGTCCTGTCTGTGGGGCAAACTTTactcagaaaggtaacctcctccgccacattaaaCTG | 46F_47F_49P_50V_52G_53A_54N_55F_67L | 2.529551253 |
| 1465 | 1307 | TTCGGTTGTGAAGTCTGTGGGGCAATTTTTactcagaaaggtaacctcctccgccacattaaaCTG | 46F_47G_49E_50V_52G_53A 54I 55F 67L | 2.528224804 |
| 1466 | 2632 | TTCATGTGTCCAGTTTGTGGGGCACCCTTTactcagaaaggtaacctcctccgccacattaaaCTG | 46F_47M_49P_50V_52G_53A_54P_55F_67L | 2.526494254 |
| 1467 | 1001 | TTCTTTTGTGACGTATGTGGGGCATGCTTTactcagaaaggtaacctcctccgccacattaaaCTG | 46F 47F 49D_50V 52G 53A 54C 55F 67L | 2.526479568 |
| 1468 | 63 | TTCGCGTGTGAAATTTGTGGGGCAGAATTTactcagaaaggtaacctcctccgccacattaaaCTG | 46F_47A_49E_50I_52G_53A_54E_55F 67L | 2.523820495 |
| 1469 | 421 | TTCTGTTGTCCAATTTGTGGGGCATGCTTTactcagaaaggtaacctcctccgccacattaaaCTG | 46F_47C_49P_50I_52G_53A_54C_55F_67L | 2.523787735 |
| 1470 | 55 | TTCGCTTGTGACGTATGTGGGGCATCCTTTactcagaaaggtaacctcctcogccacattaaaCTG | 46F_47A_49D_50V_52G_53A_54S_55F_67L | 2.523297577 |
| 1471 | 2532 | TTCATGTGTAATATTTGTGGGGCACCCTTTactcagaaaggtaacctcctccgccacattaaaCTG | 46F_47M_49N_50I_52G_53A_54P_55F_67L | 2.520078098 |
| 1472 | 3492 | TTCTCATGTAATATCTGTGGGGCACCATTTactcagaaaggtaacctcctccgccacattaaaCTG | 46F_47S_49N_50I_52G_53A 54P 55F 67L | 2.519325762 |
| 1473 | 128 | TTCGCGTGTAATATCTGTGGGGCAAAGTTTactcagaaaggtaacctcctccgccacattaaaCTG | 46F_47A_49N_50I_52G_53A 54K 55F 67L | 2.517868937 |
| 1474 | 4319 | TTCTGGTGTCCTGTTTGTGGGGCATACTTTactcagaaaggtaacctcctccgccacattaaaCTG | 46F_47W_49P_50V_52G_53A_54Y_55F_67L | 2.516394306 |
| 1475 | 6775 | CTCACCTGCCCAATCTGCGGGTTCAGCTGTactcagaaaggtaacctcctccgccacattaaaCTG | 46L_47T_49P_50I_52G_53F_54S_55C_67L | 2.513643843 |
| 1476 | 4326 | TTCTATTGTGATATTTGTGGGGCACATTTTactcagaaaggtaacctcctccgccacattaaaCTG | 46F_47Y_49D_50I_52G_53A_54H_55F_67L | 2.513206616 |
| 1477 | 6544 | CTGTTCTGCCCCATCTGCGGATTCTTTTGTactcagaaaggtaacctcctccgccacattaaaCTG | 46L_47F_49P_50I_52G_53F_54F_55C_67L | 2.504832985 |
| 1478 | 4554 | TTCTACTGTCCCGTGTGTGGGGCAAGGTTTactcagaaaggtaacctcctccgccacattaaaCTG | 46F_47Y_49P_50V_52G_53A_54R_55F_67L | 2.500814039 |
| 1479 | 3375 | TTCTCGTGTGACATCTGTGGGGCAAGCTTTactcagaaaggtaacctcctccgccacattaaaCTG | 46F 47S_49D_50I 52G_53A 54S 55F 67L | 2.500510948 |
| 1480 | 2955 | TTCCAATGTGAGATCTGTGGGGCAAGCTTTactcagaaaggtaacctcctccgccacattaaaCTG | 46F_47Q_49E_50I_52G_53A_54S_55F_67L | 2.499240554 |
| 1481 | 7934 | CTCGTCTGCGAGATTTGTGGCTTCAGATGCactcagaaaggtaacctcctccgccacattaaaCTG | 46L_47V_49E_50I_52G_53F_54R_55C_67L | 2.49822166 |
| 1482 | 1067 | TTCTTCTGTGAGGTATGTGGGGCAATATTTactcagaaaggtaacctcctccgccacattaaaCTG | 46F_47F_49E_50V_52G_53A_54I_55F_67L | 2.498000462 |
| 1483 | 4087 | TTCTGGTGTGACATTTGTGGGGCAATCTTTactcagaaaggtaacctcctccgccacattaaaCTG | 46F_47W_49D_50I_52G_53A 54I 55F 67L | 2.496011573 |
| 1484 | 6760 | CTAACCTGCCCAATCTGCGGGTTCGCTTGTactcagaaaggtaacctcctccgccacattaaaCTG | 46L 47T 49P_50I 52G_53F 54A 55C_67L | 2.493734985 |
| 1485 | 6480 | CTGTGTTGCCCCATCTGTGGCTTTGCATGCactcagaaaggtaacctcctccgccacattaaaCTG | 46L_47C_49P_50I_52G_53F_54A_55C_67L | 2.492486793 |
| 1486 | 6194 | TTTGGTTGTCCAATCTGTGGAAGACGCTTCactcagaaaggtaacctcctccgccacattaaaCTG | 46F_47G_49P_50I 52G_53R_54R_55F 67L | 2.492478322 |
| 1487 | 6199 | TTCGGTTGCCCCATTTGTGGTCGATACTTCactcagaaaggtaacctcctccgccacattaaaCTG | 46F_47G_49P_50I_52G_53R_54Y_55F_67L | 2.492201859 |
| 1488 | 7947 | TTGTGGTGTGAAATCTGCGGCTTCATTTGCactcagaaaggtaacctcctccgccacattaaaCTG | 46L_47W_49E_50I_52G_53F 54I 55C 67L | 2.491668238 |
| 1489 | 7636 | CTTTGCTGTGAGATCTGTGGTTTTACATGTactcagaaaggtaacctcctccgccacattaaaCTG | 46L_47C_49E_50I_52G_53F_54T_55C_67L | 2.491217106 |
| 1490 | 2995 | TTCCAGTGTGAGGTCTGTGGGGCATCCTTTactcagaaaggtaacctcctccgccacattaaaCTG | 46F_47Q_49E_50V_52G_53A_54S_55F_67L | 2.487922761 |
| 1491 | 4361 | TTCTACTGTGACGTTTGTGGGGCATGTTTTactcagaaaggtaacctcctccgccacattaaaCTG | 46F_47Y_49D_50V_52G_53A_54C_55F_67L | 2.48734771 |
| 1492 | 2714 | TTCAACTGTGAGATTTGTGGGGCAAGGTTTactcagaaaggtaacctcctccgccacattaaaCTG | 46F_47N_49E_50I_52G_53A_54R_55F_67L | 2.485115799 |
| 1493 | 1675 | TTCCATTGTCCAGTTTGTGGGGCATCATTTactcagaaaggtaacctcctccgccacattaaaCTG | 46F_47H_49P_50V_52G_53A_54S_55F_67L | 2.482061395 |
| 1494 | 1264 | TTCGGCTGTGAGATTTGTGGGGCATTCTTTactcagaaaggtaacctcctccgccacattaaaCTG | 46F_47G_49E_50I_52G_53A_54F_55F_67L | 2.480184564 |
| 1495 | 2753 | TTCAATTGTGAGGTGTGTGGGGCACAGTTTactcagaaaggtaacctcctccgccacattaaaCTG | 46F_47N_49E_50V_52G_53A_54Q_55F_67L | 2.476669835 |
| 1496 | 2833 | TTCAACTGTCCTATCTGTGGGGCACAGTTTactcagaaaggtaacctcctccgccacattaaaCTG | 46F_47N_49P_50I_52G_53A_54Q_55F_67L | 2.476637208 |
| 1497 | 3780 | TTCACGTGTCCAATCTGTGGGGCAGCTTTTactcagaaaggtaacctcctccgccacattaaaCTG | 46F_47T 49P_50I_52G_53A 54A 55F 67L | 2.474992218 |
| 1498 | 4263 | TTCTGGTGTCCTATTTGTGGGGCAGAGTTTactcagaaaggtaacctcctccgccacattaaaCTG | 46F_47W_49P_50I_52G_53A_54E_55F_67L | 2.46695996 |
| 1499 | 245 | TTCTGCTGTGACATTTGTGGGGCAGGGTTTactcagaaaggtaacctcctccgccacattaaaCTG | 46F_47C_49D_50I_52G_53A_54G_55F 67L | 2.466767404 |
| 1500 | 4329 | TTCTATTGTGATATCTGTGGGGCACTATTTactcagaaaggtaacctcctccgccacattaaaCTG | 46F_47Y_49D_50I_52G_53A_54L_55F_67L | 2.466492065 |
| 1501 | 3426 | TTCTCCTGTGAGATATGTGGGGCACACTTTactcagaaaggtaacctcctccgccacattaaaCTG | 46F_47S_49E_50I_52G_53A_54H_55F_67L | 2.465993387 |
| 1502 | 3551 | TTCTCATGTCCGATTTGTGGGGCAAATTTTactcagaaaggtaacctcctccgccacattaaaCTG | 46F_47S_49P_50I_52G_53A_54N_55F_67L | 2.464616707 |
| 1503 | 912 | TTCGAGTGTCCTATCTGTGGGGCACCTTTTactcagaaaggtaacctcctccgccacattaaaCTG | 46F_47E_49P_50I_52G_53A_54P_55F_67L | 2.46444817 |
| 1504 | 6705 | CTGCAGTGTCCTATCTGCGGTTTCGGCTGTactcagaaaggtaacctcctccgccacattaaaCTG | 46L_47Q_49P_50I_52G_53F_54G_55C_67L | 2.464068517 |
| 1505 | 1633 | TTCCACTGTCCGATCTGTGGGGCACAGTTTactcagaaaggtaacctcctccgccacattaaaCTG | 46F_47H_49P_50I_52G_53A 54Q_55F_67L | 2.45925075 |
| 1506 | 3439 | TTCTCCTGTGAAATCTGTGGGGCATACTTTactcagaaaggtaacctcctccgccacattaaaCTG | 46F_47S_49E_50I_52G_53A 54Y 55F_67L | 2.456843028 |
| 1507 | 4330 | TTCTATTGTGATATCTGTGGGGCAATGTTTactcagaaaggtaacctcctccgccacattaaaCTG | 46F_47Y_49D_50I_52G_53A_54M_55F_67L | 2.456030529 |
| 1508 | 6172 | TTCTTCTGCCCAATCTGCGGACGCCCATTTactcagaaaggtaacctcctccgccacattaaaCTG | 46F_47F_49P_50I_52G_53R_54P_55F_67L | 2.456005398 |
| 1509 | 468 | TTCTGTTGTCCAGTTTGTGGGGCAAAGTTTactcagaaaggtaacctcctccgccacattaaaCTG | 46F_47C_49P_50V_52G_53A_54K_55F_67L | 2.455910444 |
| 1510 | 7915 | TTGACTTGCGAGATATGTGGATTCTCCTGCactcagaaaggtaacctcctccgccacattaaaCTG | 46L_47T_49E_50I_52G_53F_54S_55C_67L | 2.447341416 |
| 1511 | 4455 | TTCTATTGTAACATCTGTGGGGCATCCTTTactcagaaaggtaacctcctccgccacattaaaCTG | 46F_47Y_49N_50I_52G_53A_54S_55F_67L | 2.446168985 |
| 1512 | 2985 | TTCCAGTGTGAGGTTTGTGGGGCAGGTTTTactcagaaaggtaacctcctccgccacattaaaCTG | 46F_47Q_49E_50V_52G_53A_54G_55F_67L | 2.44559022 |
| 1513 | 7699 | TTATTCTGCGAAATCTGCGGTTTTTATTGCactcagaaaggtaacctcctccgccacattaaaCTG | 46L_47F 49E_50I_52G_53F 54Y 55C 67L | 2.444805785 |
| 1514 | 4184 | TTCTGGTGTGAGGTGTGTGGGGCATTCTTTactcagaaaggtaacctcctccgccacattaaaCTG | 46F_47W_49E_50V_52G_53A_54F_55F_67L | 2.444134564 |
| 1515 | 222 | TTCGCATGTCCTGTGTGTGGGGCAGACTTTactcagaaaggtaacctcctccgccacattaaaCTG | 46F_47A_49P_50V_52G_53A_54D_55F_67L | 2.441306427 |
| 1516 | 10 | TTCGCTTGTGATATTTGTGGGGCAATGTTTactcagaaaggtaacctcctccgccacattaaaCTG | 46F_47A_49D_50I_52G_53A_54M_55F_67L | 2.436200876 |
| 1517 | 1208 | TTCGGGTGTGATATATGTGGGGCAAAGTTTactcagaaaggtaacctcctccgccacattaaaCTG | 46F_47G_49D_50I_52G_53A_54K 55F 67L | 2.434234943 |
| 1518 | 1414 | TTCGGCTGTCCACAGTGTGGGGCACGATTTactcagaaaggtaacctcctccgccacattaaaCTG | 46F_47G_49P_50Q_52G_53A_54R 55F 67L | 2.432526771 |
| 1519 | 6811 | CTGTGGTGTCCCATTTGCGGGTTCAATTGTactcagaaaggtaacctcctccgccacattaaaCTG | 46L_47W_49P_50I_52G_53F_54N_55C_67L | 2.43088592 |
| 1520 | 4097 | TTCTGGTGTGACATATGTGGGGCAGTGTTTactcagaaaggtaacctcctccgccacattaaaCTG | 46F_47W_49D_50I_52G_53A_54V_55F_67L | 2.428669338 |
| 1521 | 4089 | TTCTGGTGTGATATCTGTGGGGCATTATTTactcagaaaggtaacctcctccgccacattaaaCTG | 46F_47W_49D_50I_52G_53A_54L_55F_67L | 2.425521368 |
| 1522 | 3470 | TTCAGTIGTGAGGTATGTGGGGCAATGTTTactcagaaaggtaacctcctccgccacattaaaCTG | 46F_47S_49E_50V_52G_53A 54M 55F_67L | 2.425248339 |
| 1523 | 311 | TTCTGCTGTGAGATCTGTGGGGCAAATTTTactcagaaaggtaacctcctccgccacattaaaCTG | 46F_47C_49E_50I_52G_53A 54N 55F_67L | 2.424974367 |
| 1524 | 2711 | TTCAACTGTGAAATATGTGGGGCAAATTTTactcagaaaggtaacctcctccgccacattaaaCTG | 46F_47N_49E_50I_52G_53A_54N_55F 67L | 2.424048999 |
| 1525 | 6832 | TTATACTGCCCCATCTGCGGATTCCCTTGTactcagaaaggtaacctcctccgccacattaaaCTG | 46L_47Y_49P_50I_52G_53F_54P_55C_67L | 2.42082023 |
| 1526 | 3586 | TTCAGTTGTCCCGTGTGTGGGGCACATTTTactcagaaaggtaacctcctccgccacattaaaCTG | 46F_47S_49P_50V_52G_53A_54H 55F_67L | 2.417664131 |
| 1527 | 4084 | TTCTGGTGTGATATTTGTGGGGCATTCTTTactcagaaaggtaacctcctccgccacattaaaCTG | 46F_47W_49D_50I_52G_53A_54F_55F_67L | 2.415075802 |
| 1528 | 1661 | TTCCACTGTCCTGTATGTGGGGCATGTTTTactcagaaaggtaacctcctccgccacattaaaCTG | 46F_47H_49P_50V_52G_53A_54C_55F_67L | 2.414999694 |
| 1529 | 7855 | TTACAGTGCGAAATATGCGGATTCAGCTGCactcagaaaggtaacctcctccgccacattaaaCTG | 46L_47Q_49E_50I_52G_53F_54S_55C_67L | 2.412020973 |
| 1530 | 4373 | TTCTACTGTGACGTGTGTGGGGCACAGTTTactcagaaaggtaacctcctccgccacattaaaCTG | 46F_47Y_49D_50V_52G_53A_54Q_55F_67L | 2.410611969 |
| 1531 | 6180 | TTTGGATGCCCGATCTGTGGCCGCGCTTTCactcagaaaggtaacctcctccgccacattaaaCTG | 46F 47G_49P_50I_52G_53R_54A 55F_67L | 2.410090467 |
| 1532 | 4168 | TTCTGGTGTGAACAGTGTGGGGCAAAGTTTactcagaaaggtaacctcctccgccacattaaaCTG | 46F_47W_49E_50Q_52G_53A_54K_55F_67L | 2.404340081 |
| 1533 | 6092 | TTTGCATGCCCAATCTGCGGCCGGCCTTTTactcagaaaggtaacctcctccgccacattaaaCTG | 46F_47A_49P_50I_52G_53R_54P_55F_67L | 2.403676363 |
| 1534 | 4032 | TTCGTGTGTCCTATCTGTGGGGCACCTTTTactcagaaaggtaacctcctccgccacattaaaCTG | 46F_47V_49P_50I_52G_53A_54P_55F_67L | 2.403114897 |
| 1535 | 119 | TTCGCATGTGAAGTGTGTGGGGCATACTTTactcagaaaggtaacctcctccgccacattaaaCTG | 46F_47A_49E_50V_52G_53A_54Y_55F_67L | 2.401216822 |
| 1536 | 7893 | CTAAGCTGCGAGATTTGCGGGTTTCAGTGCactcagaaaggtaacctcctccgccacattaaaCTG | 46L_47S_49E_50I_52G_53F_54Q_55C_67L | 2.40098213 |
| 1537 | 6745 | TTATCTTGTCCAATTTGCGGTTTCGGGTGTactcagaaaggtaacctcctccgccacattaaaCTG | 46L_47S_49P_50I_52G_53F_54G_55C_67L | 2.398533939 |
| 1538 | 1623 | TTCCACTGTCCTATTTGTGGGGCAGAGTTTactcagaaaggtaacctcctccgccacattaaaCTG | 46F_47H_49P_50I_52G_53A_54E_55F_67L | 2.395538069 |
| 1539 | 1554 | TTCCACTGTGAGGTTTGTGGGGCACGCTTTactcagaaaggtaacctcctccgccacattaaaCTG | 46F_47H_49E_50V_52G_53A_54R_55F_67L | 2.393657922 |
| 1540 | 660 | TTCGACTGTCCTATTTGTGGGGCAGCCTTTactcagaaaggtaacctcctccgccacattaaaCTG | 46F_47D_49P_50I_52G_53A_54A_55F_67L | 2.393508898 |
| 1541 | 7733 | TTACACTGCGAGATTTGTGGCTTCCAATGCactcagaaaggtaacctcctccgccacattaaaCTG | 46L_47H_49E_50I_52G_53F_54Q_55C_67L | 2.393160333 |
| 1542 | 712 | TTCGATTGTCCGGTCTGTGGGGCACCTTTTactcagaaaggtaacctcctccgccacattaaaCTG | 46F_47D_49P_50V_52G_53A_54P_55F_67L | 2.38988564 |
| 1543 | 7935 | TTAGTGTGCGAAATCTGCGGCTTCTCCTGCactcagaaaggtaacctcctccgccacattaaaCTG | 46L_47V_49E_50I_52G_53F_54S_55C_67L | 2.389471796 |
| 1544 | 2272 | TTCCTCTGTGAAGTCTGTGGGGCACCCTTTactcagaaaggtaacctcctccgccacattaaaCTG | 46F 47L 49E_50V_52G_53A_54P_55F 67L | 2.387211689 |
| 1545 | 3705 | TTCACTTGTGAGGTGTGTGGGGCAGGTTTTactcagaaaggtaacctcctccgccacattaaaCTG | 46F_47T_49E_50V_52G_53A_54G_55F_67L | 2.386846348 |
| 1546 | 3600 | TTCACATGTGATATCTGTGGGGCAGCCTTTactcagaaaggtaacctcctccgccacattaaaCTG | 46F_47T_49D_50I_52G_53A_54A_55F_67L | 2.386329008 |
| 1547 | 433 | TTCTGCTGTCCTATTTGTGGGGCACAATTTactcagaaaggtaacctcctccgccacattaaaCTG | 46F 47C 49P 50I 52G_53A 54Q 55F 67L | 2.386088335 |
| 1548 | 4264 | TTCTGGTGTCCAATCTGTGGGGCATTCTTTactcagaaaggtaacctcctccgccacattaaaCTG | 46F_47W_49P_50I_52G_53A_54F_55F_67L | 2.386017197 |
| 1549 | 3065 | TTCCAGTGTCCCATTTGTGGGGCAGGTTTTactcagaaaggtaacctcctccgccacattaaaCTG | 46F_47Q_49P_50I_52G_53A_54G_55F_67L | 2.385696631 |
| 1550 | 6477 | CTGGCTTGCCCAATCTGCGGCTTTGTGTGTactcagaaaggtaacctcctccgccacattaaaCTG | 46L_47A_49P_50I_52G_53F_54V_55C_67L | 2.385203504 |
| 1551 | 4514 | TTCTATTGTCCGATCTGTGGGGCAAGATTTactcagaaaggtaacctcctccgccacattaaaCTG | 46F 47Y 49P 50I 52G_53A 54R 55F 67L | 2.383401274 |
| 1552 | 3195 | TTCAGGTGTGAAATCTGTGGGGCAAGCTTTactcagaaaggtaacctcctccgccacattaaaCTG | 46F 47R 49E 50I 52G_53A 54S_55F 67L | 2.380616109 |
| 1553 | 1510 | TTCCACTGTGAGATATGTGGGGCAATGTTTactcagaaaggtaacctcctccgccacattaaaCTG | 46F_47H_49E_50I_52G_53A_54M_55F_67L | 2.374945039 |
| 1554 | 461 | TTCTGCTGTCCCGTGTGTGGGGCATGTTTTactcagaaaggtaacctcctccgccacattaaaCTG | 46F_47C_49P_50V_52G_53A_54C_55F_67L | 2.37394942 |
| 1555 | 1634 | TTCCACTGTCCTATTTGTGGGGCACGTTTTactcagaaaggtaacctcctccgccacattaaaCTG | 46F_47H_49P_50I_52G_53A_54R_55F_67L | 2.37394428 |
| 1556 | 6746 | CTATCTTGCCCTATATGCGGCTTTCACTGCactcagaaaggtaacctcctccgccacattaaaCTG | 46L 47S_49P_50I_52G_53F_54H_55C_67L | 2.372293168 |
| 1557 | 7944 | CTCTGGTGCGAAATATGTGGTTTTTTTTGTactcagaaaggtaacctcctccgccacattaaaCTG | 46L_47W_49E_50I_52G_53F_54F_55C_67L | 2.370283591 |
| 1558 | 4183 | TTCTGGTGTGAAGTGTGTGGGGCAGAGTTTactcagaaaggtaacctcctccgccacattaaaCTG | 46F_47W_49E_50V_52G_53A_54E_55F_67L | 2.369103105 |
| 1559 | 3553 | TTCTCCTGTCCGATATGTGGGGCACAGTTTactcagaaaggtaacctcctccgccacattaaaCTG | 46F_47S_49P_50I_52G_53A_54Q_55F_67L | 2.368984456 |
| 1560 | 1081 | TTCTTCTGTAATATTTGTGGGGCATGCTTTactcagaaaggtaacctcctccgccacattaaaCTG | 46F_47F_49N_50I_52G_53A_54C_55F_67L | 2.365952118 |
| 1561 | 966 | TTCTTTTGTGACATTTGTGGGGCACATTTTactcagaaaggtaacctcctccgccacattaaaCTG | 46F_47F_49D_50I_52G_53A_54H_55F_67L | 2.364727251 |
| 1562 | 1492 | TTCCACTGTGATGTCTGTGGGGCACCATTTactcagaaaggtaacctcctccgccacattaaaCTG | 46F_47H_49D_50V_52G_53A_54P_55F_67L | 2.363444117 |
| 1563 | 2640 | TTCAATTGTGATATCTGTGGGGCAGCATTTactcagaaaggtaacctcctccgccacattaaaCTG | 46F_47N_49D_50I_52G_53A_54A_55F_67L | 2.361779681 |
| 1564 | 7634 | CTATGTTGCGAGATCTGTGGCTTCCGCTGTactcagaaaggtaacctcctccgccacattaaaCTG | 46L_47C_49E_50I_52G_53F_54R_55C_67L | 2.360775155 |
| 1565 | 900 | TTCGAGTGTCCAATCTGTGGGGCAGCGTTTactcagaaaggtaacctcctccgccacattaaaCTG | 46F_47E_49P_50I_52G_53A_54A 55F_67L | 2.360212834 |
| 1566 | 3372 | TTCAGCTGTGATATTTGTGGGGCACCGTTTactcagaaaggtaacctcctccgccacattaaaCTG | 46F_47S_49D_50I_52G_53A_54P_55F_67L | 2.356240114 |
| 1567 | 3665 | TTCACGTGTGAGATATGTGGGGCAGGATTTactcagaaaggtaacctcctccgccacattaaaCTG | 46F_47T_49E_50I_52G_53A_54G_55F_67L | 2.353596438 |
| 1568 | 3012 | TTCCAATGTAATATATGTGGGGCACCCTTTactcagaaaggtaacctcctccgccacattaaaCTG | 46F_47Q_49N_50I_52G_53A_54P_55F_67L | 2.353281021 |
| 1569 | 1668 | TTCCATTGTCCAGTGTGTGGGGCAAAGTTTactcagaaaggtaacctcctccgccacattaaaCTG | 46F_47H_49P_50V_52G_53A_54K_55F_67L | 2.352567402 |
| 1570 | 6584 | CTGCACTGCCCAATATGTGGCTTCTTTTGTactcagaaaggtaacctcctccgccacattaaaCTG | 46L_47H_49P_50I_52G_53F_54F_55C_67L | 2.352268523 |
| 1571 | 1015 | TTCTTTTGTGATGTGTGTGGGGCATCCTTTactcagaaaggtaacctcctccgccacattaaaCTG | 46F_47F_49D_50V_52G_53A_54S_55F_67L | 2.352072996 |
| 1572 | 3115 | TTCCAATGTCCTGTGTGTGGGGCATCATTTactcagaaaggtaacctcctccgccacattaaaCTG | 46F_47Q_49P_50V_52G_53A_54S_55F_67L | 2.349917046 |
| 1573 | 3546 | TTCAGTIGTCCAATCTGTGGGGCACACTTTactcagaaaggtaacctcctccgccacattaaaCTG | 46F_47S_49P_50I_52G_53A_54H_55F_67L | 2.349669112 |
| 1574 | 2821 | TTCAACTGTCCAATCTGTGGGGCATGCTTTactcagaaaggtaacctcctccgccacattaaaCTG | 46F_47N_49P_50I_52G_53A_54C_55F_67L | 2.344857839 |
| 1575 | 3360 | TTCAGCTGTGATATTTGTGGGGCAGCCTTTactcagaaaggtaacctcctccgccacattaaaCTG | 46F_47S_49D_50I_52G_53A_54A 55F 67L | 2.343234431 |
| 1576 | 1197 | TTCTTTTGTCCCGTCTGTGGGGCAGTCTTTactcagaaaggtaacctcctccgccacattaaaCTG | 46F_47F_49P_50V_52G_53A_54V_55F_67L | 2.342411399 |
| 1577 | 971 | TTCTTTTGTGATATCTGTGGGGCAAATTTTactcagaaaggtaacctcctccgccacattaaaCTG | 46F 47F 49D 50I_52G_53A 54N 55F 67L | 2.340998085 |
| 1578 | 254 | TTCTGTTGTGATATCTGTGGGGCACGCTTTactcagaaaggtaacctcctccgccacattaaaCTG | 46F_47C_49D_50I_52G_53A_54R_55F_67L | 2.340029208 |
| 1579 | 1085 | TTCTTCTGTAATATTTGTGGGGCAGGGTTTactcagaaaggtaacctcctccgccacattaaaCTG | 46F_47F_49N_50I_52G_53A_54G_55F_67L | 2.33835575 |
| 1580 | 4086 | TTCTGGTGTGACATATGTGGGGCACACTTTactcagaaaggtaacctcctccgccacattaaaCTG | 46F_47W_49D_50I_52G_53A_54H_55F_67L | 2.336181176 |
| 1581 | 665 | TTCGACTGTCCTATATGTGGGGCAGGCTTTactcagaaaggtaacctcctccgccacattaaaCTG | 46F 47D 49P 50I 52G 53A 54G 55F 67L | 2.334324833 |
| 1582 | 6494 | CTGTGCTGTCCTATATGCGGATTTAGGTGCactcagaaaggtaacctcctccgccacattaaaCTG | 46L_47C_49P_50I_52G_53F_54R_55C_67L | 2.330261835 |
| 1583 | 6586 | CTTCATTGCCCAATTTGCGGATTCCACTGTactcagaaaggtaacctcctccgccacattaaaCTG | 46L_47H_49P_50I_52G_53F_54H_55C_67L | 2.329117375 |
| 1584 | 3548 | TTCAGTTGTCCTATTTGTGGGGCAAAATTTactcagaaaggtaacctcctccgccacattaaaCTG | 46F_47S_49P_50I_52G_53A_54K_55F_67L | 2.327884205 |
| 1585 | 2025 | TTCAAATGTGAGGTTTGTGGGGCAGGCTTTactcagaaaggtaacctcctccgccacattaaaCTG | 46F_47K_49E_50V_52G_53A_54G_55F_67L | 2.324248032 |
| 1586 | 255 | TTCTGTTGTGACATCTGTGGGGCATCTTTTactcagaaaggtaacctcctccgccacattaaaCTG | 46F_47C_49D_50I_52G_53A_54S_55F_67L | 2.320244241 |
| 1587 | 3430 | TTCTCGTGTGAGATCTGTGGGGCAATGTTTactcagaaaggtaacctcctccgccacattaaaCTG | 46F_47S_49E_50I_52G_53A_54M_55F_67L | 2.318558657 |
| 1588 | 3345 | TTCAGATGTCCTGTCTGTGGGGCAGGCTTTactcagaaaggtaacctcctccgccacattaaaCTG | 46F_47R_49P_50V_52G_53A_54G_55F_67L | 2.316739609 |
| 1589 | 474 | TTCTGCTGTCCCGTTTGTGGGGCAAGATTTactcagaaaggtaacctcctccgccacattaaaCTG | 46F_47C_49P_50V_52G_53A_54R_55F_67L | 2.313746139 |
| 1590 | 6551 | CTGTTTTGTCCCATTTGCGGGTTCAATTGCactcagaaaggtaacctcctccgccacattaaaCTG | 46L_47F_49P_50I_52G_53F_54N_55C_67L | 2.310923891 |
| 1591 | 2580 | TTCATGTGTCCAATTTGTGGGGCAGCCTTTactcagaaaggtaacctcctccgccacattaaaCTG | 46F_47M_49P_50I_52G_53A_54A_55F_67L | 2.309459856 |
| 1592 | 6753 | CTCTCATGCCCAATTTGTGGATTCCAGTGCactcagaaaggtaacctcctccgccacattaaaCTG | 46L_47S_49P_50I_52G_53F_54Q_55C_67L | 2.307405421 |
| 1593 | 94 | TTCGCATGTGAACAGTGTGGGGCAAGATTTactcagaaaggtaacctcctccgccacattaaaCTG | 46F_47A_49E_50Q_52G_53A_54R_55F_67L | 2.305733961 |
| 1594 | 6496 | TTGTGCTGTCCGATATGTGGATTCACCTGTactcagaaaggtaacctcctccgccacattaaaCTG | 46L_47C_49P_50I_52G_53F_54T_55C_67L | 2.303478514 |
| 1595 | 4339 | TTCTACTGTGATATCTGTGGGGCATACTTTactcagaaaggtaacctcctccgccacattaaaCTG | 46F_47Y_49D_50I_52G_53A_54Y_55F_67L | 2.301568437 |
| 1596 | 1639 | TTCCACTGTCCAATCTGTGGGGCATATTTTactcagaaaggtaacctcctccgccacattaaaCTG | 46F_47H_49P_50I_52G_53A_54Y_55F_67L | 2.300955748 |
| 1597 | 6680 | CTGAACTGTCCAATCTGTGGATTCGCCTGTactcagaaaggtaacctcctccgccacattaaaCTG | 46L_47N_49P_50I_52G_53F_54A_55C_67L | 2.297760384 |
| 1598 | 3820 | TTCACCTGTCCCGTGTGTGGGGCAGCATTTactcagaaaggtaacctcctccgccacattaaaCTG | 46F_47T_49P_50V_52G_53A_54A_55F_67L | 2.297597014 |
| 1599 | 1147 | TTCTTTTGTCCCATCTGTGGGGCAATCTTTactcagaaaggtaacctcctccgccacattaaaCTG | 46F 47F 49P 50I 52G 53A 54I 55F_67L | 2.294202275 |
| 1600 | 4200 | TTCTGGTGTAACATTTGTGGGGCAGCTTTTactcagaaaggtaacctcctccgccacattaaaCTG | 46F_47W_49N_50I_52G_53A_54A_55F_67L | 2.29130007 |
| 1601 | 135 | TTCGCTTGTAATATTTGTGGGGCATCCTTTactcagaaaggtaacctcctccgccacattaaaCTG | 46F_47A_49N_50I_52G_53A_54S_55F_67L | 2.286114502 |
| 1602 | 2265 | TTCCTTTGTGAAGTGTGTGGGGCAGGCTTTactcagaaaggtaacctcctccgccacattaaaCTG | 46F_47L_49E_50V_52G_53A_54G_55F_67L | 2.278856435 |
| 1603 | 7716 | CTGGGGTGTGAGATCTGCGGGTTCACTTGCactcagaaaggtaacctcctccgccacattaaaCTG | 46L 47G_49E_50I_52G_53F_54T 55C_67L | 2.278649038 |
| 1604 | 6195 | TTCGGTTGCCCAATTTGTGGGCGGAGCTTCactcagaaaggtaacctcctccgccacattaaaCTG | 46F_47G_49P_50I_52G_53R_54S_55F_67L | 2.27843269 |
| 1605 | 310 | TTCTGCTGTGAGATTTGTGGGGCAATGTTTactcagaaaggtaacctcctccgccacattaaaCTG | 46F_47C_49E_50I_52G_53A_54M_55F_67L | 2.277362203 |
| 1606 | 6594 | TTGCATTGTCCCATCTGTGGTTTCCGTTGTactcagaaaggtaacctcctccgccacattaaaCTG | 46L_47H_49P_50I_52G_53F_54R_55C_67L | 2.275692443 |
| 1607 | 969 | TTCTTCTGTGATATCTGTGGGGCACTTTTTactcagaaaggtaacctcctccgccacattaaaCTG | 46F_47F_49D_50I_52G_53A_54L 55F 67L | 2.274887879 |
| 1608 | 668 | TTCGATTGTCCAATATGTGGGGCAAAATTTactcagaaaggtaacctcctccgccacattaaaCTG | 46F_47D_49P_50I_52G_53A_54K_55F_67L | 2.274680401 |
| 1609 | 4211 | TTCTGGTGTAATATCTGTGGGGCAAACTTTactcagaaaggtaacctcctccgccacattaaaCTG | 46F_47W_49N_50I_52G_53A_54N_55F_67L | 2.27450152 |
| 1610 | 3715 | TTCACATGTGAAGTGTGTGGGGCATCGTTTactcagaaaggtaacctcctccgccacattaaaCTG | 46F_47T_49E_50V_52G_53A_54S_55F_67L | 2.272396854 |
| 1611 | 6807 | CTTTGGTGTCCTATATGCGGCTTTATTTGCactcagaaaggtaacctcctccgccacattaaaCTG | 46L_47W_49P_50I_52G_53F_54I_55C_67L | 2.272208833 |
| 1612 | 7308 | TTTTTTTGCGAGATCTGTGGTCGGAAGTTTactcagaaaggtaacctcctccgccacattaaaCTG | 46F_47F_49E_50I_52G_53R_54K_55F_67L | 2.271906081 |
| 1613 | 3675 | TTCACATGTGAAATATGTGGGGCAAGTTTTactcagaaaggtaacctcctccgccacattaaaCTG | 46F_47T_49E_50I_52G_53A_54S_55F_67L | 2.271244609 |
| 1614 | 6569 | CTGGGGTGTCCCATATGTGGATTCCTTTGCactcagaaaggtaacctcctccgccacattaaaCTG | 46L_47G_49P_50I_52G_53F_54L_55C_67L | 2.271043931 |
| 1615 | 7840 | CTCCAGTGTGAGATATGTGGGTTTGCATGCactcagaaaggtaacctcctccgccacattaaaCTG | 46L 47Q 49E 50I_52G_53F 54A 55C_67L | 2.27027186 |
| 1616 | 103 | TTCGCCTGTGAAGTGTGTGGGGCAGAGTTTactcagaaaggtaacctcctccgccacattaaaCTG | 46F_47A_49E_50V_52G_53A_54E_55F_67L | 2.269781023 |
| 1617 | 7888 | CTGTCATGCGAGATCTGCGGCTTCAAATGTactcagaaaggtaacctcctccgccacattaaaCTG | 46L_47S_49E_50I_52G_53F_54K_55C_67L | 2.268440132 |
| 1618 | 7684 | CTGTTTTGTGAAATCTGTGGCTTTTTCTGCactcagaaaggtaacctcctccgccacattaaaCTG | 46L_47F_49E_50I_52G_53F_54F_55C_67L | 2.26823403 |
| 1619 | 3300 | TTCCGCTGTCCAATCTGTGGGGCAGCCTTTactcagaaaggtaacctcctccgccacattaaaCTG | 46F 47R 49P_50I_52G_53A_54A 55F_67L | 2.263777472 |
| 1620 | 3292 | TTCAGATGTAATGTTTGTGGGGCACCATTTactcagaaaggtaacctcctccgccacattaaaCTG | 46F_47R_49N_50V_52G_53A_54P_55F_67L | 2.262563438 |
| 1621 | 3471 | TTCTCCTGTGAAGTGTGTGGGGCAAATTTTactcagaaaggtaacctcctccgccacattaaaCTG | 46F_47S_49E_50V_52G_53A_54N_55F_67L | 2.257648509 |
| 1622 | 6795 | CTGGTGTGCCCCATCTGCGGCTTCTCTTGTactcagaaaggtaacctcctccgccacattaaaCTG | 46L_47V_49P_50I_52G_53F_54S_55C_67L | 2.247477349 |
| 1623 | 4277 | TTCTGGTGTCCGATTTGTGGGGCAGTCTTTactcagaaaggtaacctcctccgccacattaaaCTG | 46F_47W_49P_50I_52G_53A_54V_55F_67L | 2.244815331 |
| 1624 | 3593 | TTCTCGTGTCCTGTTTGTGGGGCACAGTTTactcagaaaggtaacctcctccgccacattaaaCTG | 46F_47S_49P_50V_52G_53A_54Q_55F_67L | 2.244327438 |
| 1625 | 673 | TTCGATTGTCCCATCTGTGGGGCACAATTTactcagaaaggtaacctcctccgccacattaaaCTG | 46F_47D_49P_50I_52G_53A_54Q_55F_67L | 2.24122284 |
| 1626 | 6735 | CTGCGCTGTCCCATATGTGGTTTCAGCTGTactcagaaaggtaacctcctccgccacattaaaCTG | 46L_47R_49P_50I_52G_53F_54S_55C_67L | 2.240625792 |
| 1627 | 4453 | TTCTATTGTAATATCTGTGGGGCACAGTTTactcagaaaggtaacctcctccgccacattaaaCTG | 46F_47Y_49N_50I_52G_53A_54Q_55F_67L | 2.239763103 |
| 1628 | 6696 | CTCAATTGCCCAATCTGTGGTTTTACTTGTactcagaaaggtaacctcctccgccacattaaaCTG | 46L_47N_49P_50I_52G_53F_54T_55C_67L | 2.237464726 |
| 1629 | 4099 | TTCTGGTGTGACATCTGTGGGGCATACTTTactcagaaaggtaacctcctccgccacattaaaCTG | 46F_47W_49D_50I_52G_53A_54Y_55F_67L | 2.236803396 |
| 1630 | 3559 | TTCTCCTGTCCGATCTGTGGGGCATATTTTactcagaaaggtaacctcctccgccacattaaaCTG | 46F_47S_49P_50I_52G_53A_54Y_55F_67L | 2.234767313 |
| 1631 | 7875 | CTAAGATGTGAAATCTGCGGCTTTAGTTGCactcagaaaggtaacctcctccgccacattaaaCTG | 46L_47R_49E_50I_52G_53F_54S_55C_67L | 2.2341589 |
| 1632 | 7959 | TTGTGGTGCGAGATCTGCGGCTTCTATTGCactcagaaaggtaacctcctccgccacattaaaCTG | 46L_47W_49E_50I_52G_53F_54Y_55C_67L | 2.232873518 |
| 1633 | 4304 | TTCTGGTGTCCTGTTTGTGGGGCATTTTTTactcagaaaggtaacctcctccgccacattaaaCTG | 46F_47W_49P_50V_52G_53A 54F 55F 67L | 2.232163939 |
| 1634 | 6838 | CTGTACTGCCCTATTTGTGGATTTTGGTGCactcagaaaggtaacctcctccgccacattaaaCTG | 46L_47Y_49P_50I_52G_53F_54W_55C_67L | 2.230667484 |
| 1635 | 4303 | TTCTGGTGTCCAGTCTGTGGGGCAGAATTTactcagaaaggtaacctcctccgccacattaaaCTG | 46F_47W_49P_50V_52G_53A_54E_55F_67L | 2.230528198 |
| 1636 | 4324 | TTCTACTGTGACATCTGTGGGGCATTTTTTactcagaaaggtaacctcctccgccacattaaaCTG | 46F_47Y_49D_50I_52G_53A_54F_55F_67L | 2.230008704 |
| 1637 | 2500 | TTCATGTGTGAGGTATGTGGGGCAGCTTTTactcagaaaggtaacctcctccgccacattaaaCTG | 46F 47M 49E_50V_52G_53A 54A 55F 67L | 2.229918666 |
| 1638 | 3952 | TTCGTGTGTGAGGTGTGTGGGGCACCTTTTactcagaaaggtaacctcctccgccacattaaaCTG | 46F_47V_49E_50V_52G_53A_54P_55F_67L | 2.228057042 |
| 1639 | 6471 | TTAGCCTGTCCAATCTGTGGCTTTAACTGCactcagaaaggtaacctcctccgccacattaaaCTG | 46L_47A_49P_50I_52G_53F_54N_55C_67L | 2.226211952 |
| 1640 | 3305 | TTCAGATGTCCCATTTGTGGGGCAGGCTTTactcagaaaggtaacctcctccgccacattaaaCTG | 46F_47R_49P_50I_52G_53A_54G_55F_67L | 2.225683789 |
| 1641 | 7963 | TTGTATTGCGAAATTTGCGGCTTTGAGTGCactcagaaaggtaacctcctccgccacattaaaCTG | 46L_47Y_49E_50I_52G_53F_54E_55C_67L | 2.224815411 |
| 1642 | 1000 | TTCTTTTGTGACGTGTGTGGGGCAGCGTTTactcagaaaggtaacctcctccgccacattaaaCTG | 46F_47F_49D_50V_52G_53A_54A_55F_67L | 2.219746899 |
| 1643 | 4276 | TTCTGGTGTCCCATCTGTGGGGCAACCTTTactcagaaaggtaacctcctccgccacattaaaCTG | 46F_47W_49P_50I_52G_53A_54T_55F_67L | 2.219020329 |
| 1644 | 4317 | TTCTGGTGTCCCGTCTGTGGGGCAGTTTTTactcagaaaggtaacctcctccgccacattaaaCTG | 46F_47W_49P_50V_52G_53A_54V_55F_67L | 2.218854563 |
| 1645 | 7860 | CTTCGTTGTGAAATCTGTGGCTTCGCCTGCactcagaaaggtaacctcctccgccacattaaaCTG | 46L_47R_49E_50I_52G_53F_54A_55C_67L | 2.217687349 |
| 1646 | 2645 | TTCAATTGTGATATCTGTGGGGCAGGATTTactcagaaaggtaacctcctccgccacattaaaCTG | 46F_47N_49D_50I_52G_53A_54G_55F 67L | 2.216720459 |
| 1647 | 1612 | TTCCACTGTAATGTCTGTGGGGCACCTTTTactcagaaaggtaacctcctccgccacattaaaCTG | 46F_47H_49N_50V_52G_53A_54P_55F_67L | 2.216154378 |
| 1648 | 1541 | TTCCATTGTGAGGTGTGTGGGGCATGCTTTactcagaaaggtaacctcctccgccacattaaaCTG | 46F_47H_49E_50V_52G_53A_54C_55F_67L | 2.215561621 |
| 1649 | 17 | TTCGCCTGTGATATCTGTGGGGCAGTTTTTactcagaaaggtaacctcctccgccacattaaaCTG | 46F 47A 49D_50I 52G_53A 54V 55F 67L | 2.215182206 |
| 1650 | 1679 | TTCCATTGTCCTGTGTGTGGGGCATATTTTactcagaaaggtaacctcctccgccacattaaaCTG | 46F_47H_49P_50V_52G_53A_54Y_55F_67L | 2.212494366 |
| 1651 | 3340 | TTCAGGTGTCCAGTATGTGGGGCAGCTTTTactcagaaaggtaacctcctccgccacattaaaCTG | 46F_47R_49P_50V_52G_53A_54A_55F_67L | 2.210841116 |
| 1652 | 3105 | TTCCAGTGTCCCGTGTGTGGGGCAGGCTTTactcagaaaggtaacctcctccgccacattaaaCTG | 46F_47Q_49P_50V_52G_53A_54G_55F_67L | 2.210241997 |
| 1653 | 552 | TTCGATTGTGAAATCTGTGGGGCACCTTTTactcagaaaggtaacctcctccgccacattaaaCTG | 46F_47D_49E_50I 52G_53A 54P 55F 67L | 2.206702688 |
| 1654 | 7880 | CTGTCCTGTGAGATTTGTGGTTTCGCGTGCactcagaaaggtaacctcctccgccacattaaaCTG | 46L 47S_49E_50I_52G_53F_54A 55C 67L | 2.203730299 |
| 1655 | 2839 | TTCAACTGTCCCATCTGTGGGGCATACTTTactcagaaaggtaacctcctccgccacattaaaCTG | 46F_47N_49P_50I_52G_53A 54Y 55F_67L | 2.201942013 |
| 1656 | 700 | TTCGACTGTCCGGTCTGTGGGGCAGCATTTactcagaaaggtaacctcctccgccacattaaaCTG | 46F_47D_49P_50V_52G_53A 54A 55F 67L | 2.201267125 |
| 1657 | 6472 | CTGGCCTGTCCAATCTGTGGCTTTCCATGCactcagaaaggtaacctcctccgccacattaaaCTG | 46L_47A_49P_50I_52G_53F_54P_55C_67L | 2.200884554 |
| 1658 | 2746 | TTCAATTGTGAAGTGTGTGGGGCACACTTTactcagaaaggtaacctcctccgccacattaaaCTG | 46F_47N_49E_50V_52G_53A_54H_55F_67L | 2.200745699 |
| 1659 | 223 | TTCGCCTGTCCCGTCTGTGGGGCAGAATTTactcagaaaggtaacctcctccgccacattaaaCTG | 46F_47A_49P_50V_52G_53A_54E_55F 67L | 2.197611658 |
| 1660 | 2625 | TTCATGTGTCCAGTTTGTGGGGCAGGCTTTactcagaaaggtaacctcctccgccacattaaaCTG | 46F 47M 49P 50V 52G 53A 54G 55F 67L | 2.19587986 |
| 1661 | 1080 | TTCTTCTGTAATATCTGTGGGGCAGCATTTactcagaaaggtaacctcctccgccacattaaaCTG | 46F_47F_49N_50I_52G_53A_54A_55F_67L | 2.194572872 |
| 1662 | 6588 | CTTCACTGTCCTATCTGTGGGTTCAAATGCactcagaaaggtaacctcctccgccacattaaaCTG | 46L 47H_49P_50I_52G_53F_54K_55C_67L | 2.193800019 |
| 1663 | 4214 | TTCTGGTGTAACATTTGTGGGGCAAGATTTactcagaaaggtaacctcctccgccacattaaaCTG | 46F_47W_49N_50I_52G_53A 54R 55F_67L | 2.193503306 |
| 1664 | 1184 | TTCTTCTGTCCGGTGTGTGGGGCATTTTTTactcagaaaggtaacctcctccgccacattaaaCTG | 46F_47F_49P_50V_52G_53A 54F 55F_67L | 2.193318548 |
| 1665 | 3466 | TTCTCGTGTGAGGTCTGTGGGGCACATTTTactcagaaaggtaacctcctccgccacattaaaCTG | 46F_47S_49E_50V_52G_53A_54H_55F_67L | 2.192230295 |
| 1666 | 4414 | TTCTACTGTGAGCAGTGTGGGGCACGCTTTactcagaaaggtaacctcctccgccacattaaaCTG | 46F_47Y_49E_50Q_52G_53A_54R_55F_67L | 2.191154829 |
| 1667 | 3427 | TTCAGTIGTGAAATATGTGGGGCAATTTTTactcagaaaggtaacctcctccgccacattaaaCTG | 46F_47S_49E_50I_52G_53A_54I_55F_67L | 2.191074633 |
| 1668 | 3477 | TTCTCTTGTGAGGTTTGTGGGGCAGTGTTTactcagaaaggtaacctcctccgccacattaaaCTG | 46F_47S_49E_50V_52G_53A_54V_55F 67L | 2.189802522 |
| 1669 | 1408 | TTCGGTTGTCCACAGTGTGGGGCAAAGTTTactcagaaaggtaacctcctccgccacattaaaCTG | 46F_47G_49P_50Q_52G_53A_54K 55F 67L | 2.188422712 |
| 1670 | 6831 | CTTTATTGCCCAATCTGTGGGTTTAATTGCactcagaaaggtaacctcctccgccacattaaaCTG | 46L_47Y_49P_50I_52G_53F_54N_55C_67L | 2.187647483 |
| 1671 | 6685 | TTAAACTGCCCGATCTGCGGATTCGGCTGTactcagaaaggtaacctcctccgccacattaaaCTG | 46L_47N_49P_50I_52G_53F 54G 55C_67L | 2.185894867 |
| 1672 | 7685 | CTGTTTTGCGAAATTTGTGGATTCGGATGCactcagaaaggtaacctcctccgccacattaaaCTG | 46L_47F_49E_50I_52G_53F_54G_55C_67L | 2.185760735 |
| 1673 | 2719 | TTCAACTGTGAAATCTGTGGGGCATACTTTactcagaaaggtaacctcctccgccacattaaaCTG | 46F_47N_49E_50I_52G_53A_54Y_55F_67L | 2.185065936 |
| 1674 | 1309 | TTCGGCTGTGAGGTCTGTGGGGCACTGTTTactcagaaaggtaacctcctccgccacattaaaCTG | 46F_47G_49E_50V_52G_53A_54L_55F_67L | 2.176536214 |
| 1675 | 6590 | CTCCATTGTCCGATTTGTGGCTTCATGTGTactcagaaaggtaacctcctccgccacattaaaCTG | 46L_47H_49P_50I_52G_53F_54M_55C_67L | 2.174954111 |
| 1676 | 4189 | TTCTGGTGTGAAGTTTGTGGGGCACTCTTTactcagaaaggtaacctcctccgccacattaaaCTG | 46F_47W_49E_50V_52G_53A_54L_55F_67L | 2.173712871 |
| 1677 | 4547 | TTCTACTGTCCAGTGTGTGGGGCAATCTTTactcagaaaggtaacctcctccgccacattaaaCTG | 46F_47Y_49P_50V_52G_53A_54I_55F 67L | 2.172666356 |
| 1678 | 1440 | TTCCACTGTGATATTTGTGGGGCAGCATTTactcagaaaggtaacctcctccgccacattaaaCTG | 46F_47H_49D_50I_52G_53A_54A_55F_67L | 2.171115695 |
| 1679 | 3795 | TTCACTTGTCCAATCTGTGGGGCAAGCTTTactcagaaaggtaacctcctccgccacattaaaCTG | 46F_47T_49P_50I_52G_53A_54S_55F_67L | 2.170399857 |
| 1680 | 551 | TTCGATTGTGAGATCTGTGGGGCAAATTTTactcagaaaggtaacctcctccgccacattaaaCTG | 46F_47D_49E_50I_52G_53A_54N_55F_67L | 2.167805467 |
| 1681 | 1064 | TTCTTCTGTGAGGTGTGTGGGGCATTCTTTactcagaaaggtaacctcctccgccacattaaaCTG | 46F_47F_49E_50V_52G_53A_54F_55F 67L | 2.167535351 |
| 1682 | 3712 | TTCACATGTGAGGTATGTGGGGCACCTTTTactcagaaaggtaacctcctccgccacattaaaCTG | 46F_47T_49E_50V_52G_53A_54P_55F 67L | 2.164451663 |
| 1683 | 3599 | TTCTCATGTCCCGTTTGTGGGGCATATTTTactcagaaaggtaacctcctccgccacattaaaCTG | 46F_47S 49P_50V_52G_53A 54Y 55F 67L | 2.163534757 |
| 1684 | 6720 | CTCAGATGTCCTATATGTGGTTTCGCATGCactcagaaaggtaacctcctccgccacattaaaCTG | 46L_47R_49P_50I_52G_53F_54A_55C_67L | 2.161769449 |
| 1685 | 184 | TTCGCATGTCCGATTTGTGGGGCATTCTTTactcagaaaggtaacctcctccgccacattaaaCTG | 46F_47A_49P_50I_52G_53A_54F_55F_67L | 2.158182722 |
| 1686 | 1095 | TTCTTTTGTAATATTTGTGGGGCATCCTTTactcagaaaggtaacctcctccgccacattaaaCTG | 46F_47F_49N_50I_52G_53A_54S_55F_67L | 2.157221187 |
| 1687 | 3557 | TTCAGCTGTCCCATCTGTGGGGCAGTGTTTactcagaaaggtaacctcctccgccacattaaaCTG | 46F_47S_49P_50I_52G_53A_54V 55F 67L | 2.15539854 |
| 1688 | 1214 | TTCGGGTGTGATATTTGTGGGGCAAGATTTactcagaaaggtaacctcctccgccacattaaaCTG | 46F_47G_49D_50I_52G_53A_54R 55F 67L | 2.15429165 |
| 1689 | 1980 | TTCAAATGTGAGATTTGTGGGGCAGCATTTactcagaaaggtaacctcctccgccacattaaaCTG | 46F_47K 49E_50I_52G_53A 54A 55F 67L | 2.154017846 |
| 1690 | 6748 | CTTAGTTGCCCGATCTGCGGGTTTAAATGTactcagaaaggtaacctcctccgccacattaaaCTG | 46L_47S_49P_50I_52G_53F_54K_55C_67L | 2.153751954 |
| 1691 | 6134 | TTCGATTGCCCAATTTGCGGACGTCGCTTCactcagaaaggtaacctcctccgccacattaaaCTG | 46F_47D_49P_50I_52G_53R_54R_55F_67L | 2.151311743 |
| 1692 | 915 | TTCGAGTGTCCAATCTGTGGGGCATCTTTTactcagaaaggtaacctcctccgccacattaaaCTG | 46F_47E_49P_50I_52G_53A_54S_55F_67L | 2.149218334 |
| 1693 | 1626 | TTCCACTGTCCTATTTGTGGGGCACATTTTactcagaaaggtaacctcctccgccacattaaaCTG | 46F_47H_49P_50I_52G_53A_54H_55F_67L | 2.145340764 |
| 1694 | 1445 | TTCCATTGTGACATATGTGGGGCAGGTTTTactcagaaaggtaacctcctccgccacattaaaCTG | 46F_47H_49D_50I_52G_53A_54G_55F_67L | 2.144652236 |
| 1695 | 548 | TTCGACTGTGAAATCTGTGGGGCAAAGTTTactcagaaaggtaacctcctccgccacattaaaCTG | 46F_47D_49E_50I_52G_53A_54K_55F_67L | 2.14313183 |
| 1696 | 2866 | TTCAATTGTCCAGTGTGTGGGGCACACTTTactcagaaaggtaacctcctccgccacattaaaCTG | 46F_47N_49P_50V_52G_53A_54H_55F_67L | 2.143002815 |
| 1697 | 4450 | TTCTATTGTAACATCTGTGGGGCAATGTTTactcagaaaggtaacctcctccgccacattaaaCTG | 46F_47Y_49N_50I_52G_53A_54M_55F_67L | 2.14179221 |
| 1698 | 7714 | CTGGGATGCGAGATCTGCGGCTTTAGATGCactcagaaaggtaacctcctccgccacattaaaCTG | 46L_47G_49E_50I_52G_53F_54R_55C_67L | 2.14106778 |
| 1699 | 2831 | TTCAATTGTCCTATCTGTGGGGCAAATTTTactcagaaaggtaacctcctccgccacattaaaCTG | 46F_47N_49P_50I_52G_53A_54N_55F_67L | 2.140505217 |
| 1700 | 3588 | TTCTCTTGTCCCGTGTGTGGGGCAAAGTTTactcagaaaggtaacctcctccgccacattaaaCTG | 46F_47S_49P_50V_52G_53A_54K_55F_67L | 2.137996829 |
| 1701 | 7 | TTCGCTTGTGACATCTGTGGGGCAATTTTTactcagaaaggtaacctcctccgccacattaaaCTG | 46F_47A_49D_50I_52G_53A_54I_55F_67L | 2.134409652 |
| 1702 | 2020 | TTCAAATGTGAAGTATGTGGGGCAGCTTTTactcagaaaggtaacctcctccgccacattaaaCTG | 46F_47K_49E_50V_52G_53A_54A_55F_67L | 2.13162468 |
| 1703 | 1005 | TTCTTTTGTGATGTATGTGGGGCAGGATTTactcagaaaggtaacctcctccgccacattaaaCTG | 46F_47F_49D_50V_52G_53A_54G_55F_67L | 2.131256651 |
| 1704 | 104 | TTCGCCTGTGAGGTGTGTGGGGCATTCTTTactcagaaaggtaacctcctccgccacattaaaCTG | 46F_47A_49E_50V_52G_53A_54F_55F_67L | 2.131213378 |
| 1705 | 316 | TTCTGCTGTGAGATCTGTGGGGCAACCTTTactcagaaaggtaacctcctccgccacattaaaCTG | 46F_47C_49E_50I_52G_53A_54T_55F_67L | 2.131096959 |
| 1706 | 6485 | TTATGCTGTCCCATATGTGGATTCGGCTGCactcagaaaggtaacctcctccgccacattaaaCTG | 46L_47C_49P_50I_52G_53F_54G_55C_67L | 2.126901524 |
| 1707 | 121 | TTCGCTTGTAATATTTGTGGGGCATGTTTTactcagaaaggtaacctcctccgccacattaaaCTG | 46F_47A_49N_50I_52G_53A_54C_55F_67L | 2.125555377 |
| 1708 | 2754 | TTCAACTGTGAAGTGTGTGGGGCACGCTTTactcagaaaggtaacctcctccgccacattaaaCTG | 46F_47N_49E_50V_52G_53A_54R_55F_67L | 2.123788761 |
| 1709 | 1265 | TTCGGATGTGAAATCTGTGGGGCAGGCTTTactcagaaaggtaacctcctccgccacattaaaCTG | 46F_47G_49E_50I_52G_53A_54G_55F_67L | 2.12224439 |
| 1710 | 7900 | CTAACCTGCGAGATATGTGGGTTTGCATGCactcagaaaggtaacctcctccgccacattaaaCTG | 46L_47T_49E_50I_52G_53F_54A_55C_67L | 2.118512486 |
| 1711 | 4316 | TTCTGGTGTCCAGTTTGTGGGGCAACCTTTactcagaaaggtaacctcctccgccacattaaaCTG | 46F_47W_49P_50V_52G_53A_54T_55F_67L | 2.117756978 |
| 1712 | 6803 | TTGTGGTGTCCTATTTGTGGGTTCGAGTGCactcagaaaggtaacctcctccgccacattaaaCTG | 46L_47W_49P_50I_52G_53F_54E_55C_67L | 2.117522613 |
| 1713 | 1201 | TTCGGTTGTGATATTTGTGGGGCATGTTTTactcagaaaggtaacctcctccgccacattaaaCTG | 46F_47G_49D_50I_52G_53A_54C_55F_67L | 2.117107172 |
| 1714 | 795 | TTCGAATGTGAGATTTGTGGGGCAAGCTTTactcagaaaggtaacctcctccgccacattaaaCTG | 46F_47E_49E_50I_52G_53A_54S_55F_67L | 2.115505043 |
| 1715 | 7314 | TTTTTCTGCGAGATCTGTGGTAGACGGTTCactcagaaaggtaacctcctccgccacattaaaCTG | 46F_47F_49E_50I_52G_53R_54R_55F_67L | 2.115159731 |
| 1716 | 3431 | TTCAGTTGTGAAATCTGTGGGGCAAACTTTactcagaaaggtaacctcctccgccacattaaaCTG | 46F_47S_49E_50I_52G_53A_54N_55F_67L | 2.112254769 |
| 1717 | 4401 | TTCTATTGTGAACAGTGTGGGGCATGTTTTactcagaaaggtaacctcctccgccacattaaaCTG | 46F_47Y_49E_50Q_52G_53A_54C_55F_67L | 2.109706632 |
| 1718 | 7728 | CTCCACTGTGAAATTTGTGGTTTCAAGTGCactcagaaaggtaacctcctccgccacattaaaCTG | 46L_47H_49E_50I_52G_53F_54K_55C_67L | 2.107834072 |
| 1719 | 1304 | TTCGGCTGTGAAGTCTGTGGGGCATTCTTTactcagaaaggtaacctcctccgccacattaaaCTG | 46F_47G_49E_50V_52G_53A_54F_55F_67L | 2.100285951 |
| 1720 | 2741 | TTCAATTGTGAGGTGTGTGGGGCATGCTTTactcagaaaggtaacctcctccgccacattaaaCTG | 46F_47N_49E_50V_52G_53A_54C_55F_67L | 2.09996753 |
| 1721 | 3612 | TTCACTTGTGATATCTGTGGGGCACCCTTTactcagaaaggtaacctcctccgccacattaaaCTG | 46F_47T 49D 50I_52G_53A 54P 55F 67L | 2.099830702 |
| 1722 | 2834 | TTCAATTGTCCGATTTGTGGGGCAAGGTTTactcagaaaggtaacctcctccgccacattaaaCTG | 46F_47N_49P_50I_52G_53A_54R_55F 67L | 2.099682243 |
| 1723 | 940 | TTCGAATGTCCTGTATGTGGGGCAGCATTTactcagaaaggtaacctcctccgccacattaaaCTG | 46F_47E_49P_50V_52G_53A_54A 55F 67L | 2.095788201 |
| 1724 | 2751 | TTCAATTGTGAGGTGTGTGGGGCAAATTTTactcagaaaggtaacctcctccgccacattaaaCTG | 46F_47N_49E_50V_52G_53A 54N 55F 67L | 2.093709343 |
| 1725 | 580 | TTCGACTGTGAAGTGTGTGGGGCAGCTTTTactcagaaaggtaacctcctccgccacattaaaCTG | 46F_47D_49E_50V_52G_53A_54A_55F_67L | 2.09284993 |
| 1726 | 351 | TTCTGCTGTGAAGTGTGTGGGGCAAACTTTactcagaaaggtaacctcctccgccacattaaaCTG | 46F_47C_49E_50V_52G_53A_54N 55F 67L | 2.0907306 |
| 1727 | 835 | TTCGAATGTGAAGTGTGTGGGGCATCTTTTactcagaaaggtaacctcctccgccacattaaaCTG | 46F_47E_49E_50V_52G_53A_54S_55F_67L | 2.087409748 |
| 1728 | 711 | TTCGATTGTCCAGTGTGTGGGGCAAACTTTactcagaaaggtaacctcctccgccacattaaaCTG | 46F_47D_49P_50V_52G_53A_54N_55F_67L | 2.08607044 |
| 1729 | 6212 | TTCCACTGTCCCATTTGTGGACGTCCCTTCactcagaaaggtaacctcctccgccacattaaaCTG | 46F_47H_49P_50I_52G_53R_54P_55F_67L | 2.082020473 |
| 1730 | 2105 | TTCAAATGTCCTATCTGTGGGGCAGGATTTactcagaaaggtaacctcctccgccacattaaaCTG | 46F_47K_49P_50I_52G_53A_54G_55F_67L | 2.076534012 |
| 1731 | 3365 | TTCTCCTGTGACATTTGTGGGGCAGGATTTactcagaaaggtaacctcctccgccacattaaaCTG | 46F_47S_49D_50I_52G_53A_54G_55F_67L | 2.076152784 |
| 1732 | 3235 | TTCCGATGTGAGGTGTGTGGGGCAAGCTTTactcagaaaggtaacctcctccgccacattaaaCTG | 46F_47R_49E_50V_52G_53A_54S_55F_67L | 2.07139249 |
| 1733 | 3172 | TTCAGATGTGACGTTTGTGGGGCACCGTTTactcagaaaggtaacctcctccgccacattaaaCTG | 46F_47R_49D_50V_52G_53A_54P_55F_67L | 2.068254739 |
| 1734 | 2828 | TTCAACTGTCCTATTTGTGGGGCAAAGTTTactcagaaaggtaacctcctccgccacattaaaCTG | 46F_47N_49P_50I_52G_53A_54K_55F_67L | 2.067167195 |
| 1735 | 431 | TTCTGTTGTCCCATCTGTGGGGCAAATTTTactcagaaaggtaacctcctccgccacattaaaCTG | 46F_47C_49P_50I_52G_53A_54N_55F_67L | 2.065965276 |
| 1736 | 6185 | TTTGGCTGTCCAATCTGTGGCCGGGGTTTCactcagaaaggtaacctcctccgccacattaaaCTG | 46F_47G_49P_50I_52G_53R_54G_55F_67L | 2.065571485 |
| 1737 | 4376 | TTCTACTGTGACGTTTGTGGGGCAACCTTTactcagaaaggtaacctcctccgccacattaaaCTG | 46F_47Y_49D_50V_52G_53A 54T 55F 67L | 2.063283938 |
| 1738 | 2152 | TTCAAATGTCCTGTATGTGGGGCACCCTTTactcagaaaggtaacctcctccgccacattaaaCTG | 46F_47K_49P_50V_52G_53A_54P_55F_67L | 2.056749276 |
| 1739 | 7713 | CTGGGTTGTGAGATCTGCGGCTTCCAGTGTactcagaaaggtaacctcctccgccacattaaaCTG | 46L 47G_49E_50I_52G_53F_54Q_55C_67L | 2.056507516 |
| 1740 | 306 | TTCTGCTGTGAAATTTGTGGGGCACACTTTactcagaaaggtaacctcctccgccacattaaaCTG | 46F_47C_49E_50I_52G_53A_54H_55F_67L | 2.055392023 |
| 1741 | 6744 | CTCTCCTGTCCAATATGTGGCTTTTTTTGCactcagaaaggtaacctcctccgccacattaaaCTG | 46L 47S_49P_50I_52G_53F_54F_55C_67L | 2.048537298 |
| 1742 | 553 | TTCGACTGTGAGATATGTGGGGCACAGTTTactcagaaaggtaacctcctccgccacattaaaCTG | 46F_47D_49E_50I_52G_53A_54Q_55F_67L | 2.043501101 |
| 1743 | 174 | TTCGCCTGTAACGTGTGTGGGGCAAGATTTactcagaaaggtaacctcctccgccacattaaaCTG | 46F_47A 49N_50V_52G_53A 54R 55F 67L | 2.043052209 |
| 1744 | 2100 | TTCAAGTGTCCCATATGTGGGGCAGCATTTactcagaaaggtaacctcctccgccacattaaaCTG | 46F_47K_49P_50I_52G_53A_54A_55F_67L | 2.042369015 |
| 1745 | 62 | TTCGCCTGTGAGATCTGTGGGGCAGATTTTactcagaaaggtaacctcctccgccacattaaaCTG | 46F_47A_49E_50I_52G_53A_54D_55F_67L | 2.040065833 |
| 1746 | 7617 | CTCGCCTGCGAAATATGCGGTTTTGTCTGCactcagaaaggtaacctcctccgccacattaaaCTG | 46L_47A_49E_50I_52G_53F_54V_55C_67L | 2.039182133 |
| 1747 | 6467 | CTGGCTTGTCCCATTTGTGGTTTTATTTGTactcagaaaggtaacctcctccgccacattaaaCTG | 46L_47A_49P_50I_52G_53F_54I_55C_67L | 2.037690503 |
| 1748 | 6493 | CTGTGTTGTCCGATCTGCGGCTTTCAGTGCactcagaaaggtaacctcctccgccacattaaaCTG | 46L 47C_49P_50I_52G_53F_54Q_55C_67L | 2.035462959 |
| 1749 | 133 | TTCGCCTGTAACATTTGTGGGGCACAGTTTactcagaaaggtaacctcctccgccacattaaaCTG | 46F_47A_49N_50I_52G_53A_54Q_55F_67L | 2.035445021 |
| 1750 | 3479 | TTCAGCTGTGAAGTGTGTGGGGCATATTTTactcagaaaggtaacctcctccgccacattaaaCTG | 46F_47S_49E_50V_52G_53A_54Y_55F_67L | 2.033987477 |
| 1751 | 3732 | TTCACATGTAATATCTGTGGGGCACCCTTTactcagaaaggtaacctcctccgccacattaaaCTG | 46F 47T 49N 50I 52G 53A 54P 55F 67L | 2.033738391 |
| 1752 | 1792 | TTCATCTGTGAGGTGTGTGGGGCACCATTTactcagaaaggtaacctcctccgccacattaaaCTG | 46F 47I 49E_50V_52G_53A 54P 55F 67L | 2.029603485 |
| 1753 | 4210 | TTCTGGTGTAATATTTGTGGGGCAATGTTTactcagaaaggtaacctcctccgccacattaaaCTG | 46F_47W_49N_50I_52G_53A_54M_55F 67L | 2.029302215 |
| 1754 | 6583 | CTACACTGTCCAATCTGCGGATTTGAGTGTactcagaaaggtaacctcctccgccacattaaaCTG | 46L_47H_49P_50I_52G_53F_54E_55C_67L | 2.028246346 |
| 1755 | 6552 | CTCTTTTGCCCCATCTGTGGATTTCCATGTactcagaaaggtaacctcctccgccacattaaaCTG | 46L 47F_49P_50I_52G_53F_54P 55C 67L | 2.023282721 |
| 1756 | 2874 | TTCAACTGTCCCGTCTGTGGGGCACGCTTTactcagaaaggtaacctcctccgccacattaaaCTG | 46F_47N_49P_50V_52G_53A_54R_55F_67L | 2.022550318 |
| 1757 | 4213 | TTCTGGTGTAACATCTGTGGGGCACAGTTTactcagaaaggtaacctcctccgccacattaaaCTG | 46F_47W_49N_50I_52G_53A_54Q_55F_67L | 2.022525656 |
| 1758 | 2352 | TTCTTATGTCCCATCTGTGGGGCACCCTTTactcagaaaggtaacctcctccgccacattaaaCTG | 46F_47L_49P_50I_52G_53A 54P 55F_67L | 2.021719998 |
| 1759 | 2220 | TTCCTCTGTGAGATATGTGGGGCAGCGTTTactcagaaaggtaacctcctccgccacattaaaCTG | 46F_47L_49E_50I_52G_53A_54A_55F_67L | 2.021533988 |
| 1760 | 7865 | TTGAGATGTGAAATTTGTGGCTTCGGTTGCactcagaaaggtaacctcctccgccacattaaaCTG | 46L 47R_49E_50I_52G_53F_54G_55C_67L | 2.018858678 |
| 1761 | 2032 | TTCAAGTGTGAGGTGTGTGGGGCACCTTTTactcagaaaggtaacctcctccgccacattaaaCTG | 46F_47K_49E_50V_52G_53A_54P 55F 67L | 2.015717044 |
| 1762 | 1093 | TTCTTCTGTAACATTTGTGGGGCACAATTTactcagaaaggtaacctcctccgccacattaaaCTG | 46F 47F_49N_50I_52G_53A 54Q 55F 67L | 2.015402022 |
| 1763 | 214 | TTCGCCTGTCCTCAATGTGGGGCACGTTTTactcagaaaggtaacctcctccgccacattaaaCTG | 46F_47A_49P_50Q_52G_53A_54R_55F_67L | 2.01161088 |
| 1764 | 6432 | TTTTGGTGCCCAATTTGTGGCCGCCCATTTactcagaaaggtaacctcctccgccacattaaaCTG | 46F_47W_49P_50I_52G_53R_54P_55F_67L | 2.011114825 |
| 1765 | 2873 | TTCAATTGTCCCGTGTGTGGGGCACAGTTTactcagaaaggtaacctcctccgccacattaaaCTG | 46F_47N_49P_50V_52G_53A_54Q_55F_67L | 2.010389145 |
| 1760 | 7601 | TTGGCATGTGAGATATGCGGTTTTTGTTGCactcagaaaggtaacctcctccgccacattaaaCTG | 46L_47A_49E_50I_52G_53F_54C_55C_67L | 2.006228222 |
| 1767 | 1448 | TTCCATTGTGACATTTGTGGGGCAAAATTTactcagaaaggtaacctcctccgccacattaaaCTG | 46F_47H_49D_50I_52G_53A_54K_55F_67L | 2.00469557 |
| 1768 | 7971 | CTCTACTGTGAAATTTGTGGATTCAATTGTactcagaaaggtaacctcctccgccacattaaaCTG | 46L_47Y_49E_50I_52G_53F_54N_55C_67L | 2.004596121 |
| 1769 | 4130 | TTCTGGTGTGATGTGTGTGGGGCAATGTTTactcagaaaggtaacctcctccgccacattaaaCTG | 46F_47W_49D_50V_52G_53A_54M_55F_67L | 2.004001358 |
| 1770 | 7628 | CTCTGCTGTGAGATCTGTGGGTTTAAATGTactcagaaaggtaacctcctccgccacattaaaCTG | 46L_47C_49E_50I_52G_53F_54K_55C_67L | 2.000652851 |

TABLE 3
B08 orthogonality; Estimated LogFC Coefficient calculated from the limma voom linear regression contrast of the positive vs negative read
counts for each individual mutant degron as defined relative to SEQ ID NO: 2.

| | | | Annotation of amino acids according | |
|---|---|---|---|---|
| | | | to their position in SEQ ID NO: 2, | |
| | | | wherein the stated one letter | |
| | | | amino acid may be the original | |
| | | DNA | amino acid found in SEQ ID NO: 2 or a | |
| | | sequence encoding positions F146-L167 of SEQ ID NO: 2 absent any | substitution (first numerical digit | |
| SEQ ID | Degron- | substitutions defined according to the encoding sequence below and | dropped, i.e. position 146 in | |
| NO | ID | the variant amino acid sequence set out in column 4 of this Table | SEQ ID NO: 2 = 46 as stated below) | B08_logFC |
| 1771 | 4155 | TTCTGGTGTGAGATTTGTGGGGCATCTTTTactcagaaaggtaacctcctccgccacattaaaCTG | 46F_47W_49E_50I_52G_53A_54S_55F_67L | 4.449438937 |
| 1772 | 4091 | TTCTGGTGTGACATTTGTGGGGCAAACTTTactcagaaaggtaacctcctccgccacattaaaCTG | 46F_47W_49D_50I_52G_53A_54N_55F_67L | 4.276180962 |
| 1773 | 4213 | TTCTGGTGTAACATCTGTGGGGCACAGTTTactcagaaaggtaacctcctccgccacattaaaCTG | 46F_47W_49N_50I_52G_53A_54Q_55F_67L | 4.274781832 |
| 1774 | 4080 | TTCTGGTGTGATATCTGTGGGGCAGCCTTTactcagaaaggtaacctcctccgccacattaaaCTG | 46F_47W_49D_50I_52G_53A_54A_55F_67L | 4.260051396 |
| 1775 | 4195 | TTCTGGTGTGAGGTGTGTGGGGCATCCTTTactcagaaaggtaacctcctccgccacattaaaCTG | 46F_47W_49E_50V_52G_53A_54S_55F_67L | 4.256668042 |
| 1776 | 4133 | TTCTGGTGTGACGTTTGTGGGGCACAGTTTactcagaaaggtaacctcctccgccacattaaaCTG | 46F_47W_49D_50V_52G_53A_54Q_55F_67L | 4.236869123 |
| 1777 | 4435 | TTCTACTGTGAAGTTTGTGGGGCAAGCTTTactcagaaaggtaacctcctccgccacattaaaCTG | 46F_47Y_49E_50V_52G_53A_54S_55F_67L | 4.235604256 |
| 1778 | 4095 | TTCTGGTGTGATATCTGTGGGGCAAGTTTTactcagaaaggtaacctcctccgccacattaaaCTG | 46F_47W_49D_50I_52G_53A_54S_55F_67L | 4.219861332 |
| 1779 | 4135 | TTCTGGTGTGACGTCTGTGGGGCATCTTTTactcagaaaggtaacctcctccgccacattaaaCTG | 46F_47W_49D_50V_52G_53A_54S_55F_67L | 4.206711678 |
| 1780 | 4313 | TTCTGGTGTCCAGTGTGTGGGGCACAGTTTactcagaaaggtaacctcctccgccacattaaaCTG | 46F_47W_49P_50V_52G_53A_54Q_55F_67L | 4.197454461 |
| 1781 | 4175 | TTCTGGTGTGAGCAGTGTGGGGCATCCTTTactcagaaaggtaacctcctccgccacattaaaCTG | 46F_47W_49E_50Q_52G_53A_54S_55F_67L | 4.195240976 |
| 1782 | 4146 | TTCTGGTGTGAGATCTGTGGGGCACACTTTactcagaaaggtaacctcctccgccacattaaaCTG | 46F_47W_49E_50I_52G_53A_54H_55F_67L | 4.18611121 |
| 1783 | 4141 | TTCTGGTGTGAGATCTGTGGGGCATGCTTTactcagaaaggtaacctcctccgccacattaaaCTG | 46F_47W_49E_50I_52G_53A_54C_55F_67L | 4.182985596 |
| 1784 | 4396 | TTCTACTGTGAGATCTGTGGGGCAACATTTactcagaaaggtaacctcctccgccacattaaaCTG | 46F_47Y_49E_50I_52G_53A_54T_55F_67L | 4.175479709 |
| 1785 | 4120 | TTCTGGTGTGACGTCTGTGGGGCAGCATTTactcagaaaggtaacctcctccgccacattaaaCTG | 46F_47W_49D_50V_52G_53A_54A_55F_67L | 4.174550793 |
| 1786 | 4148 | TTCTGGTGTGAAATCTGTGGGGCAAAGTTTactcagaaaggtaacctcctccgccacattaaaCTG | 46F_47W_49E_50I_52G_53A_54K_55F_67L | 4.169205204 |
| 1787 | 4423 | TTCTATTGTGAAGTTTGTGGGGCAGAGTTTactcagaaaggtaacctcctccgccacattaaaCTG | 46F_47Y_49E_50V_52G_53A_54E_55F_67L | 4.141665143 |
| 1788 | 4395 | TTCTATTGTGAGATCTGTGGGGCATCTTTTactcagaaaggtaacctcctccgccacattaaaCTG | 46F_47Y_49E_50I_52G_53A_54S_55F_67L | 4.137477656 |
| 1789 | 4255 | TTCTGGTGTAACGTCTGTGGGGCATCTTTTactcagaaaggtaacctcctccgccacattaaaCTG | 46F_47W_49N_50V_52G_53A_54S_55F_67L | 4.13566263 |
| 1790 | 4193 | TTCTGGTGTGAGGTGTGTGGGGCACAGTTTactcagaaaggtaacctcctccgccacattaaaCTG | 46F_47W_49E_50V_52G_53A_54Q_55F_67L | 4.134357689 |
| 1791 | 4311 | TTCTGGTGTCCCGTCTGTGGGGCAAATTTTactcagaaaggtaacctcctccgccacattaaaCTG | 46F_47W_49P_50V_52G_53A_54N_55F_67L | 4.129949239 |
| 1792 | 4196 | TTCTGGTGTGAAGTTTGTGGGGCAACATTTactcagaaaggtaacctcctccgccacattaaaCTG | 46F_47W_49E_50V_52G_53A_54T_55F_67L | 4.1239529 |
| 1793 | 4121 | TTCTGGTGTGATGTATGTGGGGCATGCTTTactcagaaaggtaacctcctccgccacattaaaCTG | 46F_47W_49D_50V_52G_53A_54C_55F_67L | 4.113253999 |
| 1794 | 4197 | TTCTGGTGTGAGGTGTGTGGGGCAGTCTTTactcagaaaggtaacctcctccgccacattaaaCTG | 46F_47W_49E_50V_52G_53A_54V_55F_67L | 4.112544552 |
| 1795 | 4211 | TTCTGGTGTAATATCTGTGGGGCAAACTTTactcagaaaggtaacctcctccgccacattaaaCTG | 46F_47W_49N_50I_52G_53A_54N_55F_67L | 4.105222963 |
| 1796 | 4231 | TTCTGGTGTAACCAATGTGGGGCAAACTTTactcagaaaggtaacctcctccgccacattaaaCTG | 46F_47W_49N_50Q_52G_53A_54N_55F_67L | 4.104043452 |
| 1797 | 4096 | TTCTGGTGTGATATCTGTGGGGCAACATTTactcagaaaggtaacctcctccgccacattaaaCTG | 46F_47W_49D_50I_52G_53A_54T_55F_67L | 4.100600845 |
| 1798 | 4181 | TTCTGGTGTGAAGTCTGTGGGGCATGCTTTactcagaaaggtaacctcctccgccacattaaaCTG | 46F_47W_49E_50V_52G_53A_54C_55F_67L | 4.096237523 |
| 1799 | 1031 | TTCTTCTGTGAGATTTGTGGGGCAAATTTTactcagaaaggtaacctcctccgccacattaaaCTG | 46F_47F_49E_50I_52G_53A_54N_55F_67L | 4.088330008 |
| 1800 | 7955 | CTGTGGTGCGAGATCTGCGGCTTCAGCTGTactcagaaaggtaacctcctccgccacattaaaCTG | 46L_47W_49E_50I_52G_53F_54S_55C_67L | 4.086511198 |
| 1801 | 4275 | TTCTGGTGTCCGATCTGTGGGGCAAGTTTTactcagaaaggtaacctcctccgccacattaaaCTG | 46F_47W_49P_50I_52G_53A_54S_55F_67L | 4.086487622 |
| 1802 | 4391 | TTCTACTGTGAAATTTGTGGGGCAAATTTTactcagaaaggtaacctcctccgccacattaaaCTG | 46F_47Y_49E_50I_52G_53A_54N_55F_67L | 4.07609494 |
| 1803 | 4140 | TTCTGGTGTGAAATTTGTGGGGCAGCTTTTactcagaaaggtaacctcctccgccacattaaaCTG | 46F_47W_49E_50I_52G_53A_54A_55F_67L | 4.073568307 |
| 1804 | 4241 | TTCTGGTGTAATGTGTGTGGGGCATGTTTTactcagaaaggtaacctcctccgccacattaaaCTG | 46F_47W_49N_50V_52G_53A_54C_55F_67L | 4.068744743 |
| 1805 | 7956 | CTCTGGTGTGAAATTTGCGGATTCACCTGCactcagaaaggtaacctcctccgccacattaaaCTG | 46L_47W_49E_50I_52G_53F_54T_55C_67L | 4.064426599 |
| 1806 | 4251 | TTCTGGTGTAATGTATGTGGGGCAAATTTTactcagaaaggtaacctcctccgccacattaaaCTG | 46F_47W_49N_50V_52G_53A_54N_55F_67L | 4.061395242 |
| 1807 | 4199 | TTCTGGTGTGAGGTCTGTGGGGCATACTTTactcagaaaggtaacctcctccgccacattaaaCTG | 46F_47W_49E_50V_52G_53A_54Y_55F_67L | 4.060454694 |
| 1808 | 4420 | TTCTACTGTGAAGTCTGTGGGGCAGCTTTTactcagaaaggtaacctcctccgccacattaaaCTG | 46F_47Y_49E_50V_52G_53A_54A_55F_67L | 4.060261325 |
| 1809 | 4153 | TTCTGGTGTGAAATTTGTGGGGCACAGTTTactcagaaaggtaacctcctccgccacattaaaCTG | 46F_47W_49E_50I_52G_53A_54Q_55F_67L | 4.05831085 |
| 1810 | 4291 | TTCTGGTGTCCCCAGTGTGGGGCAAACTTTactcagaaaggtaacctcctccgccacattaaaCTG | 46F_47W_49P_50Q_52G_53A_54N_55F_67L | 4.047803602 |
| 1811 | 4301 | TTCTGGTGTCCGGTTTGTGGGGCATGCTTTactcagaaaggtaacctcctccgccacattaaaCTG | 46F_47W_49P_50V_52G_53A_54C_55F_67L | 4.043793173 |
| 1812 | 4126 | TTCTGGTGTGATGTCTGTGGGGCACACTTTactcagaaaggtaacctcctccgccacattaaaCTG | 46F_47W_49D_50V_52G_53A_54H_55F_67L | 4.036067525 |
| 1813 | 4437 | TTCTACTGTGAGGTGTGTGGGGCAGTGTTTactcagaaaggtaacctcctccgccacattaaaCTG | 46F_47Y_49E_50V_52G_53A_54V_55F_67L | 4.034209695 |
| 1814 | 4300 | TTCTGGTGTCCAGTGTGTGGGGCAGCATTTactcagaaaggtaacctcctccgccacattaaaCTG | 46F_47W_49P_50V_52G_53A_54A_55F_67L | 4.027607236 |
| 1815 | 4156 | TTCTGGTGTGAAATCTGTGGGGCAACGTTTactcagaaaggtaacctcctccgccacattaaaCTG | 46F_47W_49E_50I_52G_53A_54T_55F_67L | 4.024101375 |
| 1816 | 1075 | TTCTTTTGTGAAGTCTGTGGGGCAAGCTTTactcagaaaggtaacctcctccgccacattaaaCTG | 46F_47F_49E_50V_52G_53A_54S_55F_67L | 4.02283338 |
| 1817 | 7976 | CTCTATTGCGAAATATGCGGTTTCACATGCactcagaaaggtaacctcctccgccacattaaaCTG | 46L_47Y_49E_50I_52G_53F_54T_55C_67L | 4.012500316 |
| 1818 | 4191 | TTCTGGTGTGAGGTTTGTGGGGCAAACTTTactcagaaaggtaacctcctccgccacattaaaCTG | 46F_47W_49E_50V_52G_53A_54N_55F_67L | 4.010793364 |
| 1819 | 6813 | CTGTGGTGTCCGATCTGCGGCTTTCAATGCactcagaaaggtaacctcctccgccacattaaaCTG | 46L_47W_49P_50I_52G_53F_54Q_55C_67L | 4.008514505 |
| 1820 | 4157 | TTCTGGTGTGAAATTTGTGGGGCAGTATTTactcagaaaggtaacctcctccgccacattaaaCTG | 46F_47W_49E_50I_52G_53A_54V_55F_67L | 4.006844345 |
| 1821 | 4147 | TTCTGGTGTGAGATTTGTGGGGCAATATTTactcagaaaggtaacctcctccgccacattaaaCTG | 46F_47W_49E_50I_52G_53A_54I_55F_67L | 4.000995539 |
| 1822 | 1021 | TTCTTTTGTGAAATCTGTGGGGCATGCTTTactcagaaaggtaacctcctccgccacattaaaCTG | 46F_47F_49E_50I_52G_53A_54C_55F_67L | 3.995110951 |
| 1823 | 4194 | TTCTGGTGTGAGGTTTGTGGGGCACGCTTTactcagaaaggtaacctcctccgccacattaaaCTG | 46F_47W_49E_50V_52G_53A_54R_55F_67L | 3.98851874 |
| 1824 | 4081 | TTCTGGTGTGATATCTGTGGGGCATGCTTTactcagaaaggtaacctcctccgccacattaaaCTG | 46F_47W_49D_50I_52G_53A_54C_55F_67L | 3.985538392 |
| 1825 | 4390 | TTCTACTGTGAGATATGTGGGGCAATGTTTactcagaaaggtaacctcctccgccacattaaaCTG | 46F_47Y_49E_50I_52G_53A_54M_55F_67L | 3.982788588 |
| 1826 | 4333 | TTCTACTGTGACATTTGTGGGGCACAGTTTactcagaaaggtaacctcctccgccacattaaaCTG | 46F_47Y_49D_50I_52G_53A_54Q_55F_67L | 3.982599699 |
| 1827 | 4186 | TTCTGGTGTGAGGTGTGTGGGGCACATTTTactcagaaaggtaacctcctccgccacattaaaCTG | 46F_47W_49E_50V_52G_53A_54H_55F_67L | 3.981328498 |
| 1828 | 4128 | TTCTGGTGTGATGTCTGTGGGGCAAAGTTTactcagaaaggtaacctcctccgccacattaaaCTG | 46F_47W_49D_50V_52G_53A_54K_55F_67L | 3.980555404 |
| 1829 | 4331 | TTCTATTGTGATATTTGTGGGGCAAACTTTactcagaaaggtaacctcctccgccacattaaaCTG | 46F_47Y_49D_50I_52G_53A_54N_55F_67L | 3.955481814 |
| 1830 | 4381 | TTCTATTGTGAGATATGTGGGGCATGCTTTactcagaaaggtaacctcctccgccacattaaaCTG | 46F_47Y_49E_50I_52G_53A_54C_55F_67L | 3.952794448 |
| 1831 | 4271 | TTCTGGTGTCCCATCTGTGGGGCAAACTTTactcagaaaggtaacctcctccgccacattaaaCTG | 46F_47W_49P_50I_52G_53A_54N_55F_67L | 3.950224144 |
| 1832 | 4421 | TTCTACTGTGAGGTGTGTGGGGCATGCTTTactcagaaaggtaacctcctccgccacattaaaCTG | 46F_47Y_49E_50V_52G_53A_54C_55F_67L | 3.948311703 |
| 1833 | 4093 | TTCTGGTGTGACATCTGTGGGGCACAGTTTactcagaaaggtaacctcctccgccacattaaaCTG | 46F_47W_49D_50I_52G_53A_54Q_55F_67L | 3.943881558 |
| 1834 | 4214 | TTCTGGTGTAACATTTGTGGGGCAAGATTTactcagaaaggtaacctcctccgccacattaaaCTG | 46F_47W_49N_50I_52G_53A_54R_55F_67L | 3.943844091 |
| 1835 | 4383 | TTCTACTGTGAAATCTGTGGGGCAGAGTTTactcagaaaggtaacctcctccgccacattaaaCTG | 46F_47Y_49E_50I_52G_53A_54E_55F_67L | 3.930644606 |
| 1836 | 4099 | TTCTGGTGTGACATCTGTGGGGCATACTTTactcagaaaggtaacctcctccgccacattaaaCTG | 46F_47W_49D_50I_52G_53A_54Y_55F_67L | 3.929074545 |
| 1837 | 4143 | TTCTGGTGTGAGATTTGTGGGGCAGAGTTTactcagaaaggtaacctcctccgccacattaaaCTG | 46F_47W_49E_50I_52G_53A_54E_55F_67L | 3.922781402 |
| 1838 | 4111 | TTCTGGTGTGACCAATGTGGGGCAAACTTTactcagaaaggtaacctcctccgccacattaaaCTG | 46F_47W_49D_50Q_52G_53A_54N_55F_67L | 3.918939402 |
| 1839 | 4183 | TTCTGGTGTGAAGTGTGTGGGGCAGAGTTTactcagaaaggtaacctcctccgccacattaaaCTG | 46F_47W_49E_50V_52G_53A_54E_55F_67L | 3.915545127 |
| 1840 | 6836 | CTCTATTGTCCCATTTGTGGGTTCACTTGCactcagaaaggtaacctcctccgccacattaaaCTG | 46L_47Y_49P_50I_52G_53F_54T_55C_67L | 3.915115475 |
| 1841 | 4145 | TTCTGGTGTGAAATCTGTGGGGCAGGCTTTactcagaaaggtaacctcctccgccacattaaaCTG | 46F_47W_49E_50I_52G_53A_54G_55F_67L | 3.914571683 |
| 1842 | 4185 | TTCTGGTGTGAAGTGTGTGGGGCAGGATTTactcagaaaggtaacctcctccgccacattaaaCTG | 46F_47W_49E_50V_52G_53A_54G_55F_67L | 3.913328688 |
| 1843 | 4276 | TTCTGGTGTCCCATCTGTGGGGCAACCTTTactcagaaaggtaacctcctccgccacattaaaCTG | 46F_47W_49P_50I_52G_53A_54T_55F_67L | 3.912370952 |
| 1844 | 7695 | CTTTTCTGCGAAATTTGTGGCTTCTCCTGCactcagaaaggtaacctcctccgccacattaaaCTG | 46L_47F_49E_50I_52G_53F_54S_55C_67L | 3.912112217 |
| 1845 | 4086 | TTCTGGTGTGACATATGTGGGGCACACTTTactcagaaaggtaacctcctccgccacattaaaCTG | 46F_47W_49D_50I_52G_53A_54H_55F_67L | 3.899428788 |
| 1846 | 4150 | TTCTGGTGTGAGATATGTGGGGCAATGTTTactcagaaaggtaacctcctccgccacattaaaCTG | 46F_47W_49E_50I_52G_53A_54M_55F_67L | 3.898582942 |
| 1847 | 6816 | CTGTGGTGTCCCATCTGCGGCTTCACTTGTactcagaaaggtaacctcctccgccacattaaaCTG | 46L_47W_49P_50I_52G_53F_54T_55C_67L | 3.897848719 |
| 1848 | 4137 | TTCTGGTGTGACGTGTGTGGGGCAGTCTTTactcagaaaggtaacctcctccgccacattaaaCTG | 46F_47W_49D_50V_52G_53A_54V_55F_67L | 3.896972819 |
| 1849 | 4235 | TTCTGGTGTAACCAGTGTGGGGCAAGTTTTactcagaaaggtaacctcctccgccacattaaaCTG | 46F_47W_49N_50Q_52G_53A_54S_55F_67L | 3.896093373 |
| 1850 | 4261 | TTCTGGTGTCCAATCTGTGGGGCATGCTTTactcagaaaggtaacctcctccgccacattaaaCTG | 46F_47W_49P_50I_52G_53A_54C_55F_67L | 3.894461398 |
| 1851 | 4215 | TTCTGGTGTAATATATGTGGGGCAAGCTTTactcagaaaggtaacctcctccgccacattaaaCTG | 46F_47W_49N_50I_52G_53A_54S_55F_67L | 3.894306492 |
| 1852 | 4260 | TTCTGGTGTCCCATATGTGGGGCAGCTTTTactcagaaaggtaacctcctccgccacattaaaCTG | 46F_47W_49P_50I_52G_53A_54A_55F_67L | 3.892437699 |
| 1853 | 4431 | TTCTATTGTGAGGTATGTGGGGCAAACTTTactcagaaaggtaacctcctccgccacattaaaCTG | 46F_47Y_49E_50V_52G_53A_54N_55F_67L | 3.891625776 |
| 1854 | 4316 | TTCTGGTGTCCAGTTTGTGGGGCAACCTTTactcagaaaggtaacctcctccgccacattaaaCTG | 46F_47W_49P_50V_52G_53A_54T_55F_67L | 3.891051793 |
| 1855 | 4201 | TTCTGGTGTAACATCTGTGGGGCATGTTTTactcagaaaggtaacctcctccgccacattaaaCTG | 46F_47W_49N_50I_52G_53A_54C_55F_67L | 3.891007758 |
| 1856 | 3312 | TTCCGCTGTCCCATCTGTGGGGCACCATTTactcagaaaggtaacctcctccgccacattaaaCTG | 46F_47R_49P_50I_52G_53A_54P_55F_67L | 3.887885077 |
| 1857 | 4216 | TTCTGGTGTAATATCTGTGGGGCAACATTTactcagaaaggtaacctcctccgccacattaaaCTG | 46F_47W_49N_50I_52G_53A_54T_55F_67L | 3.88549492 |
| 1858 | 4387 | TTCTACTGTGAAATTTGTGGGGCAATCTTTactcagaaaggtaacctcctccgccacattaaaCTG | 46F_47Y_49E_50I_52G_53A_54I_55F_67L | 3.882803584 |
| 1859 | 4253 | TTCTGGTGTAACGTATGTGGGGCACAGTTTactcagaaaggtaacctcctccgccacattaaaCTG | 46F_47W_49N_50V_52G_53A_54Q_55F_67L | 3.880656863 |
| 1860 | 4159 | TTCTGGTGTGAAATTTGTGGGGCATACTTTactcagaaaggtaacctcctccgccacattaaaCTG | 46F_47W_49E_50I_52G_53A_54Y_55F_67L | 3.880012037 |
| 1861 | 4397 | TTCTACTGTGAGATATGTGGGGCAGTATTTactcagaaaggtaacctcctccgccacattaaaCTG | 46F_47Y_49E_50I_52G_53A_54V_55F_67L | 3.879422686 |
| 1862 | 4160 | TTCTGGTGTGAGCAGTGTGGGGCAGCTTTTactcagaaaggtaacctcctccgccacattaaaCTG | 46F_47W_49E_50Q_52G_53A_54A_55F_67L | 3.879373806 |
| 1863 | 4436 | TTCTACTGTGAGGTTTGTGGGGCAACGTTTactcagaaaggtaacctcctccgccacattaaaCTG | 46F_47Y_49E_50V_52G_53A_54T_55F_67L | 3.875831081 |
| 1864 | 4180 | TTCTGGTGTGAGGTCTGTGGGGCAGCCTTTactcagaaaggtaacctcctccgccacattaaaCTG | 46F_47W_49E_50V_52G_53A_54A_55F_67L | 3.87574282 |
| 1865 | 1061 | TTCTTCTGTGAGGTGTGTGGGGCATGCTTTactcagaaaggtaacctcctccgccacattaaaCTG | 46F_47F_49E_50V_52G_53A_54C_55F_67L | 3.867343676 |
| 1866 | 7957 | CTGTGGTGCGAGATTTGTGGATTTGTATGCactcagaaaggtaacctcctccgccacattaaaCTG | 46L_47W_49E_50I_52G_53F_54V_55C_67L | 3.861812066 |
| 1867 | 4392 | TTCTATTGTGAAATTTGTGGGGCACCATTTactcagaaaggtaacctcctccgccacattaaaCTG | 46F_47Y_49E_50I_52G_53A_54P_55F_67L | 3.856966773 |
| 1868 | 4273 | TTCTGGTGTCCAATTTGTGGGGCACAGTTTactcagaaaggtaacctcctccgccacattaaaCTG | 46F_47W_49P_50I_52G_53A_540_55F_67L | 3.854932554 |
| 1869 | 4136 | TTCTGGTGTGATGTCTGTGGGGCAACGTTTactcagaaaggtaacctcctccgccacattaaaCTG | 46F_47W_49D_50V_52G_53A_54T_55F_67L | 3.851668527 |
| 1870 | 4427 | TTCTATTGTGAAGTTTGTGGGGCAATCTTTactcagaaaggtaacctcctccgccacattaaaCTG | 46F_47Y_49E_50V_52G_53A_54I_55F_67L | 3.849039959 |
| 1871 | 7975 | CTCTACTGCGAGATTTGCGGGTTTAGCTGCactcagaaaggtaacctcctccgccacattaaaCTG | 46L_47Y_49E_50I_52G_53F_54S_55C_67L | 3.843496296 |
| 1872 | 4240 | TTCTGGTGTAATGTCTGTGGGGCAGCCTTTactcagaaaggtaacctcctccgccacattaaaCTG | 46F_47W_49N_50V_52G_53A_54A_55F_67L | 3.839518397 |
| 1873 | 4516 | TTCTATTGTCCAATCTGTGGGGCAACATTTactcagaaaggtaacctcctccgccacattaaaCTG | 46F_47Y_49P_50I_52G_53A_54T_55F_67L | 3.839009913 |
| 1874 | 4161 | TTCTGGTGTGAGCAGTGTGGGGCATGCTTTactcagaaaggtaacctcctccgccacattaaaCTG | 46F_47W_49E_50Q_52G_53A_54C_55F_67L | 3.835439438 |
| 1875 | 4210 | TTCTGGTGTAATATTTGTGGGGCAATGTTTactcagaaaggtaacctcctccgccacattaaaCTG | 46F_47W_49N_50I_52G_53A_54M_55F_67L | 3.834289371 |
| 1876 | 4217 | TTCTGGTGTAATATCTGTGGGGCAGTCTTTactcagaaaggtaacctcctccgccacattaaaCTG | 46F_47W_49N_50I_52G_53A_54V_55F_67L | 3.831783985 |
| 1877 | 7696 | CTGTTCTGTGAGATTTGTGGCTTCACGTGCactcagaaaggtaacctcctccgccacattaaaCTG | 46L_47F_49E_50I_52G_53F_54T_55C_67L | 3.82528054 |
| 1878 | 1060 | TTCTTCTGTGAGGTTTGTGGGGCAGCTTTTactcagaaaggtaacctcctccgccacattaaaCTG | 46F_47F_49E_50V_52G_53A_54A_55F_67L | 3.822382638 |
| 1879 | 4380 | TTCTATTGTGAAATCTGTGGGGCAGCCTTTactcagaaaggtaacctcctccgccacattaaaCTG | 46F_47Y_49E_50I_52G_53A_54A_55F_67L | 3.820778517 |
| 1880 | 4131 | TTCTGGTGTGACGTTTGTGGGGCAAATTTTactcagaaaggtaacctcctccgccacattaaaCTG | 46F_47W_49D_50V_52G_53A_54N_55F_67L | 3.818557069 |
| 1881 | 7973 | CTCTATTGCGAGATCTGTGGATTTCAGTGTactcagaaaggtaacctcctccgccacattaaaCTG | 46L_47Y_49E_50I_52G_53F_54Q_55C_67L | 3.818464231 |
| 1882 | 4315 | TTCTGGTGTCCCGTATGTGGGGCATCTTTTactcagaaaggtaacctcctccgccacattaaaCTG | 46F_47W_49P_50V_52G_53A_54S_55F_67L | 3.817318848 |
| 1883 | 1063 | TTCTTCTGTGAGGTATGTGGGGCAGAGTTTactcagaaaggtaacctcctccgccacattaaaCTG | 46F_47F_49E_50V_52G_53A_54E_55F_67L | 3.814919772 |
| 1884 | 6835 | TTGTACTGCCCTATATGTGGGTTCAGCTGCactcagaaaggtaacctcctccgccacattaaaCTG | 46L_47Y_49P_50I_52G_53F_54S_55C_67L | 3.806521928 |
| 1885 | 973 | TTCTTTTGTGACATTTGTGGGGCACAATTTactcagaaaggtaacctcctccgccacattaaaCTG | 46F_47F_49D_50I_52G_53A_54Q_55F_67L | 3.802105697 |
| 1886 | 4187 | TTCTGGTGTGAAGTCTGTGGGGCAATCTTTactcagaaaggtaacctcctccgccacattaaaCTG | 46F_47W_49E_50V_52G_53A_54I_55F_67L | 3.797599837 |
| 1887 | 4094 | TTCTGGTGTGATATCTGTGGGGCACGCTTTactcagaaaggtaacctcctccgccacattaaaCTG | 46F_47W_49D_50I_52G_53A_54R_55F_67L | 3.79593066 |
| 1888 | 1752 | TTCATCTGTGAGATTTGTGGGGCACCTTTTactcagaaaggtaacctcctccgccacattaaaCTG | 46F_47I_49E_50I_52G_53A_54P_55F_67L | 3.790934721 |
| 1889 | 4386 | TTCTACTGTGAGATTTGTGGGGCACACTTTactcagaaaggtaacctcctccgccacattaaaCTG | 46F_47Y_49E_50I_52G_53A_54H_55F_67L | 3.790615127 |
| 1890 | 4154 | TTCTGGTGTGAAATTTGTGGGGCACGTTTTactcagaaaggtaacctcctccgccacattaaaCTG | 46F_47W_49E_50I_52G_53A_54R_55F_67L | 3.790142127 |
| 1891 | 7954 | CTTTGGTGTGAAATCTGCGGATTCAGATGTactcagaaaggtaacctcctccgccacattaaaCTG | 46L_47W_49E_50I_52G_53F_54R_55C_67L | 3.788734622 |
| 1892 | 4151 | TTCTGGTGTGAGATATGTGGGGCAAATTTTactcagaaaggtaacctcctccgccacattaaaCTG | 46F_47W_49E_50I_52G_53A_54N_55F_67L | 3.788170744 |
| 1893 | 4513 | TTCTACTGTCCTATCTGTGGGGCACAGTTTactcagaaaggtaacctcctccgccacattaaaCTG | 46F_47Y_49P_50I_52G_53A_54Q_55F_67L | 3.787619487 |
| 1894 | 4206 | TTCTGGTGTAATATCTGTGGGGCACACTTTactcagaaaggtaacctcctccgccacattaaaCTG | 46F_47W_49N_50I_52G_53A_54H_55F_67L | 3.784240069 |
| 1895 | 4293 | TTCTGGTGTCCTCAGTGTGGGGCACAGTTTactcagaaaggtaacctcctccgccacattaaaCTG | 46F_47W_49P_50Q_52G_53A_54Q_55F_67L | 3.784233592 |
| 1896 | 820 | TTCGAATGTGAAGTGTGTGGGGCAGCATTTactcagaaaggtaacctcctccgccacattaaaCTG | 46F_47E_49E_50V_52G_53A_54A_55F_67L | 3.781020929 |
| 1897 | 4115 | TTCTGGTGTGACCAATGTGGGGCATCATTTactcagaaaggtaacctcctccgccacattaaaCTG | 46F_47W_49D_50Q_52G_53A_54S_55F_67L | 3.778938472 |
| 1898 | 4200 | TTCTGGTGTAACATTTGTGGGGCAGCTTTTactcagaaaggtaacctcctccgccacattaaaCTG | 46F_47W_49N_50I_52G_53A_54A_55F_67L | 3.778660659 |
| 1899 | 7961 | TTGTACTGTGAGATCTGCGGATTCTGTTGTactcagaaaggtaacctcctccgccacattaaaCTG | 46L_47Y_49E_50I_52G_53F_54C_55C_67L | 3.777287882 |
| 1900 | 4274 | TTCTGGTGTCCTATTTGTGGGGCACGATTTactcagaaaggtaacctcctccgccacattaaaCTG | 46F_47W_49P_50I_52G_53A_54R_55F_67L | 3.76885278 |
| 1901 | 4097 | TTCTGGTGTGACATATGTGGGGCAGTGTTTactcagaaaggtaacctcctccgccacattaaaCTG | 46F_47W_49D_50I_52G_53A_54V_55F_67L | 3.76872724 |
| 1902 | 4388 | TTCTACTGTGAGATCTGTGGGGCAAAATTTactcagaaaggtaacctcctccgccacattaaaCTG | 46F_47Y_49E_50I_52G_53A_54K_55F_67L | 3.766779793 |
| 1903 | 4212 | TTCTGGTGTAATATCTGTGGGGCACCATTTactcagaaaggtaacctcctccgccacattaaaCTG | 46F_47W_49N_50I_52G_53A_54P_55F_67L | 3.765946258 |
| 1904 | 4393 | TTCTACTGTGAGATCTGTGGGGCACAATTTactcagaaaggtaacctcctccgccacattaaaCTG | 46F_47Y_49E_50I_52G_53A_54Q_55F_67L | 3.764167983 |
| 1905 | 4130 | TTCTGGTGTGATGTGTGTGGGGCAATGTTTactcagaaaggtaacctcctccgccacattaaaCTG | 46F_47W_49D_50V_52G_53A_54M_55F_67L | 3.759721633 |
| 1906 | 4306 | TTCTGGTGTCCAGTCTGTGGGGCACACTTTactcagaaaggtaacctcctccgccacattaaaCTG | 46F_47W_49P_50V_52G_53A_54H_55F_67L | 3.755649742 |
| 1907 | 6555 | TTGTTTTGTCCTATATGCGGGTTTTCATGCactcagaaaggtaacctcctccgccacattaaaCTG | 46L_47F_49P_50I_52G_53F_54S_55C_67L | 3.747873676 |
| 1908 | 4171 | TTCTGGTGTGAGCAGTGTGGGGCAAACTTTactcagaaaggtaacctcctccgccacattaaaCTG | 46F_47W_49E_50Q_52G_53A_54N_55F_67L | 3.747556185 |
| 1909 | 4511 | TTCTATTGTCCCATTTGTGGGGCAAACTTTactcagaaaggtaacctcctccgccacattaaaCTG | 46F_47Y_49P_50I_52G_53A_54N_55F_67L | 3.744626122 |
| 1910 | 4332 | TTCTATTGTGATATATGTGGGGCACCGTTTactcagaaaggtaacctcctccgccacattaaaCTG | 46F_47Y_49D_50I_52G_53A_54P_55F_67L | 3.742187175 |
| 1911 | 1037 | TTCTTTTGTGAGATCTGTGGGGCAGTGTTTactcagaaaggtaacctcctccgccacattaaaCTG | 46F_47F_49E_50I_52G_53A_54V_55F_67L | 3.7378415 |
| 1912 | 4335 | TTCTACTGTGATATTTGTGGGGCAAGCTTTactcagaaaggtaacctcctccgccacattaaaCTG | 46F_47Y_49D_50I_52G_53A_54S_55F_67L | 3.737491024 |
| 1913 | 3232 | TTCAGGTGTGAGGTTTGTGGGGCACCGTTTactcagaaaggtaacctcctccgccacattaaaCTG | 46F_47R_49E_50V_52G_53A_54P_55F_67L | 3.732511075 |
| 1914 | 1073 | TTCTTCTGTGAGGTCTGTGGGGCACAGTTTactcagaaaggtaacctcctccgccacattaaaCTG | 46F_47F_49E_50V_52G_53A_54Q_55F_67L | 3.731660669 |
| 1915 | 4089 | TTCTGGTGTGATATCTGTGGGGCATTATTTactcagaaaggtaacctcctccgccacattaaaCTG | 46F_47W_49D_50I_52G_53A_54L_55F_67L | 3.731217709 |
| 1916 | 1020 | TTCTTCTGTGAGATCTGTGGGGCAGCTTTTactcagaaaggtaacctcctccgccacattaaaCTG | 46F_47F_49E_50I_52G_53A_54A_55F_67L | 3.72928269 |
| 1917 | 4320 | TTCTACTGTGACATATGTGGGGCAGCCTTTactcagaaaggtaacctcctccgccacattaaaCTG | 46F_47Y_49D_50I_52G_53A_54A_55F_67L | 3.729020362 |
| 1918 | 6800 | CTATGGTGCCCCATCTGCGGCTTTGCCTGCactcagaaaggtaacctcctccgccacattaaaCTG | 46L_47W_49P_50I_52G_53F_54A_55C_67L | 3.7097174 |
| 1919 | 7953 | CTGTGGTGTGAGATTTGTGGCTTCCAGTGCactcagaaaggtaacctcctccgccacattaaaCTG | 46L_47W_49E_50I_52G_53F_54Q_55C_67L | 3.708853904 |
| 1920 | 1035 | TTCTTTTGTGAGATCTGTGGGGCAAGCTTTactcagaaaggtaacctcctccgccacattaaaCTG | 46F_47F_49E_50I_52G_53A_54S_55F_67L | 3.706871158 |
| 1921 | 6815 | CTGTGGTGCCCGATCTGTGGATTTAGCTGCactcagaaaggtaacctcctccgccacattaaaCTG | 46L_47W_49P_50I_52G_53F_54S_55C_67L | 3.704206428 |
| 1922 | 4426 | TTCTACTGTGAGGTGTGTGGGGCACATTTTactcagaaaggtaacctcctccgccacattaaaCTG | 46F_47Y_49E_50V_52G_53A_54H_55F_67L | 3.704007815 |
| 1923 | 1028 | TTCTTTTGTGAGATTTGTGGGGCAAAATTTactcagaaaggtaacctcctccgccacattaaaCTG | 46F_47F_49E_50I_52G_53A_54K_55F_67L | 3.702222675 |
| 1924 | 1076 | TTCTTCTGTGAGGTGTGTGGGGCAACATTTactcagaaaggtaacctcctccgccacattaaaCTG | 46F_47F_49E_50V_52G_53A_54T_55F_67L | 3.699517491 |
| 1925 | 4373 | TTCTACTGTGACGTGTGTGGGGCACAGTTTactcagaaaggtaacctcctccgccacattaaaCTG | 46F_47Y_49D_50V_52G_53A_54Q_55F_67L | 3.688546774 |
| 1926 | 4173 | TTCTGGTGTGAGCAGTGTGGGGCACAATTTactcagaaaggtaacctcctccgccacattaaaCTG | 46F_47W_49E_50Q_52G_53A_54Q_55F_67L | 3.684487883 |
| 1927 | 6556 | CTGTTTTGTCCCATCTGCGGGTTCACATGTactcagaaaggtaacctcctccgccacattaaaCTG | 46L_47F_49P_50I_52G_53F_54T_55C_67L | 3.681470086 |
| 1928 | 961 | TTCTTTTGTGATATCTGTGGGGCATGTTTTactcagaaaggtaacctcctccgccacattaaaCTG | 46F_47F_49D_50I_52G_53A_54C_55F_67L | 3.679904874 |
| 1929 | 7697 | CTGTTCTGCGAGATTTGCGGATTTGTCTGCactcagaaaggtaacctcctccgccacattaaaCTG | 46L_47F_49E_50I_52G_53F_54V_55C_67L | 3.678411954 |
| 1930 | 7940 | TTATGGTGTGAGATCTGTGGCTTTGCCTGTactcagaaaggtaacctcctccgccacattaaaCTG | 46L_47W_49E_50I_52G_53F_54A_55C_67L | 3.675754451 |
| 1931 | 7960 | CTGTACTGTGAGATTTGCGGATTCGCTTGTactcagaaaggtaacctcctccgccacattaaaCTG | 46L_47Y_49E_50I_52G_53F_54A_55C_67L | 3.671290027 |
| 1932 | 4190 | TTCTGGTGTGAAGTGTGTGGGGCAATGTTTactcagaaaggtaacctcctccgccacattaaaCTG | 46F_47W_49E_50V_52G_53A_54M_55F_67L | 3.667060545 |
| 1933 | 1036 | TTCTTTTGTGAAATTTGTGGGGCAACTTTTactcagaaaggtaacctcctccgccacattaaaCTG | 46F_47F_49E_50I_52G_53A_54T_55F_67L | 3.66566238 |
| 1934 | 6817 | CTCTGGTGCCCCATCTGCGGATTCGTGTGCactcagaaaggtaacctcctccgccacattaaaCTG | 46L_47W_49P_50I_52G_53F_54V_55C_67L | 3.665268385 |
| 1935 | 1023 | TTCTTCTGTGAAATATGTGGGGCAGAGTTTactcagaaaggtaacctcctccgccacattaaaCTG | 46F_47F_49E_50I_52G_53A_54E_55F_67L | 3.664692994 |
| 1936 | 4184 | TTCTGGTGTGAGGTGTGTGGGGCATTCTTTactcagaaaggtaacctcctccgccacattaaaCTG | 46F_47W_49E_50V_52G_53A_54F_55F_67L | 3.663194853 |
| 1937 | 6806 | CTGTGGTGTCCTATCTGCGGGTTCCACTGTactcagaaaggtaacctcctccgccacattaaaCTG | 46L_47W_49P_50I_52G_53F_54H_55C_67L | 3.66218786 |
| 1938 | 1193 | TTCTTCTGTCCAGTGTGTGGGGCACAGTTTactcagaaaggtaacctcctccgccacattaaaCTG | 46F_47F_49P_50V_52G_53A_54Q_55F_67L | 3.661564355 |
| 1939 | 4433 | TTCTATTGTGAGGTTTGTGGGGCACAATTTactcagaaaggtaacctcctccgccacattaaaCTG | 46F_47Y_49E_50V_52G_53A_54Q_55F_67L | 3.65605915 |
| 1940 | 4219 | TTCTGGTGTAATATCTGTGGGGCATATTTTactcagaaaggtaacctcctccgccacattaaaCTG | 46F_47W_49N_50I_52G_53A_54Y_55F_67L | 3.656054889 |
| 1941 | 4321 | TTCTACTGTGACATTTGTGGGGCATGTTTTactcagaaaggtaacctcctccgccacattaaaCTG | 46F_47Y_49D_50I_52G_53A_54C_55F_67L | 3.651701413 |
| 1942 | 4336 | TTCTACTGTGACATCTGTGGGGCAACTTTTactcagaaaggtaacctcctccgccacattaaaCTG | 46F_47Y_49D_50I_52G_53A_54T_55F_67L | 3.64843122 |
| 1943 | 4551 | TTCTACTGTCCAGTCTGTGGGGCAAATTTTactcagaaaggtaacctcctccgccacattaaaCTG | 46F_47Y_49P_50V_52G_53A_54N_55F_67L | 3.64769285 |
| 1944 | 4144 | TTCTGGTGTGAAATTTGTGGGGCATTCTTTactcagaaaggtaacctcctccgccacattaaaCTG | 46F_47W_49E_50I_52G_53A_54F_55F_67L | 3.64499644 |
| 1945 | 3352 | TTCCGCTGTCCCGTTTGTGGGGCACCATTTactcagaaaggtaacctcctccgccacattaaaCTG | 46F_47R_49P_50V_52G_53A_54P_55F_67L | 3.644694578 |
| 1946 | 1155 | TTCTTCTGTCCCATCTGTGGGGCATCCTTTactcagaaaggtaacctcctccgccacattaaaCTG | 46F_47F_49P_50I_52G_53A_54S_55F_67L | 3.643770416 |
| 1947 | 7693 | TTATTCTGTGAGATCTGCGGTTTTCAGTGTactcagaaaggtaacctcctccgccacattaaaCTG | 46L_47F_49E_50I_52G_53F_54Q_55C_67L | 3.64325548 |
| 1948 | 4434 | TTCTACTGTGAGGTCTGTGGGGCAAGGTTTactcagaaaggtaacctcctccgccacattaaaCTG | 46F_47Y_49E_50V_52G_53A_54R_55F_67L | 3.643033085 |
| 1949 | 6801 | CTCTGGTGTCCCATTTGTGGCTTTTGCTGCactcagaaaggtaacctcctccgccacattaaaCTG | 46L_47W_49P_50I_52G_53F_54C_55C_67L | 3.642649089 |
| 1950 | 1153 | TTCTTTTGTCCTATTTGTGGGGCACAGTTTactcagaaaggtaacctcctccgccacattaaaCTG | 46F_47F_49P_50I_52G_53A_54Q_55F_67L | 3.64126956 |
| 1951 | 4277 | TTCTGGTGTCCGATTTGTGGGGCAGTCTTTactcagaaaggtaacctcctccgccacattaaaCTG | 46F_47W_49P_50I_52G_53A_54V_55F_67L | 3.63866176 |
| 1952 | 4188 | TTCTGGTGTGAAGTGTGTGGGGCAAAATTTactcagaaaggtaacctcctccgccacattaaaCTG | 46F_47W_49E_50V_52G_53A_54K_55F_67L | 3.637755828 |
| 1953 | 7950 | CTCTGGTGTGAGATATGCGGGTTTATGTGCactcagaaaggtaacctcctccgccacattaaaCTG | 46L_47W_49E_50I_52G_53F_54M_55C_67L | 3.633627092 |
| 1954 | 4440 | TTCTATTGTAATATCTGTGGGGCAGCCTTTactcagaaaggtaacctcctccgccacattaaaCTG | 46F_47Y_49N_50I_52G_53A_54A_55F_67L | 3.632467389 |
| 1955 | 6837 | CTATACTGCCCGATATGCGGGTTTGTCTGTactcagaaaggtaacctcctccgccacattaaaCTG | 46L_47Y_49P_50I_52G_53F_54V_55C_67L | 3.63135513 |
| 1956 | 1196 | TTCTTCTGTCCTGTGTGTGGGGCAACTTTTactcagaaaggtaacctcctccgccacattaaaCTG | 46F_47F_49P_50V_52G_53A_54T_55F_67L | 3.631109199 |
| 1957 | 1013 | TTCTTTTGTGACGTTTGTGGGGCACAATTTactcagaaaggtaacctcctccgccacattaaaCTG | 46F_47F_49D_50V_52G_53A_54Q_55F_67L | 3.629856218 |
| 1958 | 4134 | TTCTGGTGTGACGTGTGTGGGGCACGCTTTactcagaaaggtaacctcctccgccacattaaaCTG | 46F_47W_49D_50V_52G_53A_54R_55F_67L | 3.628608793 |
| 1959 | 1515 | TTCCACTGTGAGATTTGTGGGGCATCTTTTactcagaaaggtaacctcctccgccacattaaaCTG | 46F_47H_49E_50I_52G_53A_54S_55F_67L | 3.627760246 |
| 1960 | 4208 | TTCTGGTGTAACATCTGTGGGGCAAAGTTTactcagaaaggtaacctcctccgccacattaaaCTG | 46F_47W_49N_50I_52G_53A_54K_55F_67L | 3.625540558 |
| 1961 | 7977 | CTGTACTGCGAAATTTGCGGCTTTGTGTGTactcagaaaggtaacctcctccgccacattaaaCTG | 46L_47Y_49E_50I_52G_53F_54V_55C_67L | 3.621574501 |
| 1962 | 4272 | TTCTGGTGTCCCATCTGTGGGGCACCGTTTactcagaaaggtaacctcctccgccacattaaaCTG | 46F_47W_49P_50I_52G_53A_54P_55F_67L | 3.621421116 |
| 1963 | 555 | TTCGATTGTGAGATTTGTGGGGCATCCTTTactcagaaaggtaacctcctccgccacattaaaCTG | 46F_47D_49E_50I_52G_53A_54S_55F_67L | 3.616793634 |
| 1964 | 1026 | TTCTTTTGTGAGATCTGTGGGGCACACTTTactcagaaaggtaacctcctccgccacattaaaCTG | 46F_47F_49E_50I_52G_53A_54H_55F_67L | 3.615222818 |
| 1965 | 4090 | TTCTGGTGTGACATTTGTGGGGCAATGTTTactcagaaaggtaacctcctccgccacattaaaCTG | 46F_47W_49D_50I_52G_53A_54M_55F_67L | 3.613790609 |
| 1966 | 6811 | CTGTGGTGTCCCATTTGCGGGTTCAATTGTactcagaaaggtaacctcctccgccacattaaaCTG | 46L_47W_49P_50I_52G_53F_54N_55C_67L | 3.610388936 |
| 1967 | 4428 | TTCTATTGTGAAGTATGTGGGGCAAAGTTTactcagaaaggtaacctcctccgccacattaaaCTG | 46F_47Y_49E_50V_52G_53A_54K_55F_67L | 3.609076874 |
| 1968 | 4314 | TTCTGGTGTCCTGTGTGTGGGGCAAGATTTactcagaaaggtaacctcctccgccacattaaaCTG | 46F_47W_49P_50V_52G_53A_54R_55F_67L | 3.608359161 |
| 1969 | 4295 | TTCTGGTGTCCTCAATGTGGGGCAAGTTTTactcagaaaggtaacctcctccgccacattaaaCTG | 46F_47W_49P_50Q_52G_53A_54S_55F_67L | 3.605796059 |
| 1970 | 7941 | CTATGGTGCGAAATATGCGGATTCTGTTGCactcagaaaggtaacctcctccgccacattaaaCTG | 46L_47W_49E_50I_52G_53F_54C_55C_67L | 3.604408962 |
| 1971 | 4429 | TTCTATTGTGAAGTGTGTGGGGCACTGTTTactcagaaaggtaacctcctccgccacattaaaCTG | 46F_47Y_49E_50V_52G_53A_54L_55F_67L | 3.603529972 |
| 1972 | 4268 | TTCTGGTGTCCCATCTGTGGGGCAAAGTTTactcagaaaggtaacctcctccgccacattaaaCTG | 46F_47W_49P_50I_52G_53A_54K_55F_67L | 3.601536833 |
| 1973 | 6810 | CTGTGGTGCCCCATTTGTGGCTTCATGTGTactcagaaaggtaacctcctccgccacattaaaCTG | 46L_47W_49P_50I_52G_53F_54M_55C_67L | 3.601124303 |
| 1974 | 1077 | TTCTTCTGTGAGGTGTGTGGGGCAGTGTTTactcagaaaggtaacctcctccgccacattaaaCTG | 46F_47F_49E_50V_52G_53A_54V_55F_67L | 3.598186324 |
| 1975 | 4553 | TTCTACTGTCCGGTGTGTGGGGCACAATTTactcagaaaggtaacctcctccgccacattaaaCTG | 46F_47Y_49P_50V_52G_53A_54Q_55F_67L | 3.596233039 |
| 1976 | 4246 | TTCTGGTGTAACGTGTGTGGGGCACACTTTactcagaaaggtaacctcctccgccacattaaaCTG | 46F_47W_49N_50V_52G_53A_54H_55F_67L | 3.594885746 |
| 1977 | 4375 | TTCTATTGTGATGTCTGTGGGGCATCCTTTactcagaaaggtaacctcctccgccacattaaaCTG | 46F_47Y_49D_50V_52G_53A_54S_55F_67L | 3.593920686 |
| 1978 | 6833 | CTCTACTGCCCTATTTGCGGGTTCCAGTGCactcagaaaggtaacctcctccgccacattaaaCTG | 46L_47Y_49P_50I_52G_53F_54Q_55C_67L | 3.593589288 |
| 1979 | 4149 | TTCTGGTGTGAAATCTGTGGGGCACTGTTTactcagaaaggtaacctcctccgccacattaaaCTG | 46F_47W_49E_50I_52G_53A_54L_55F_67L | 3.59325215 |
| 1980 | 580 | TTCGACTGTGAAGTGTGTGGGGCAGCTTTTactcagaaaggtaacctcctccgccacattaaaCTG | 46F_47D_49E_50V_52G_53A_54A_55F_67L | 3.588564206 |
| 1981 | 6814 | CTTTGGTGCCCAATATGTGGCTTCCGGTGCactcagaaaggtaacctcctccgccacattaaaCTG | 46L_47W_49P_50I_52G_53F_54R_55C_67L | 3.586121888 |
| 1982 | 4556 | TTCTATTGTCCGGTGTGTGGGGCAACTTTTactcagaaaggtaacctcctccgccacattaaaCTG | 46F_47Y_49P_50V_52G_53A_54T_55F_67L | 3.584538604 |
| 1983 | 1156 | TTCTTTTGTCCCATATGTGGGGCAACGTTTactcagaaaggtaacctcctccgccacattaaaCTG | 46F_47F_49P_50I_52G_53A_54T_55F_67L | 3.58301741 |
| 1984 | 2952 | TTCCAGTGTGAGATTTGTGGGGCACCATTTactcagaaaggtaacctcctccgccacattaaaCTG | 46F_47Q_49E_50I_52G_53A_54P_55F_67L | 3.58083426 |
| 1985 | 4088 | TTCTGGTGTGATATTTGTGGGGCAAAGTTTactcagaaaggtaacctcctccgccacattaaaCTG | 46F_47W_49D_50I_52G_53A_54K_55F_67L | 3.580577654 |
| 1986 | 4360 | TTCTACTGTGACGTCTGTGGGGCAGCGTTTactcagaaaggtaacctcctccgccacattaaaCTG | 46F_47Y_49D_50V_52G_53A_54A_55F_67L | 3.57818296 |
| 1987 | 1071 | TTCTTCTGTGAGGTCTGTGGGGCAAACTTTactcagaaaggtaacctcctccgccacattaaaCTG | 46F_47F_49E_50V_52G_53A_54N_55F_67L | 3.577314074 |
| 1988 | 4142 | TTCTGGTGTGAAATTTGTGGGGCAGATTTTactcagaaaggtaacctcctccgccacattaaaCTG | 46F_47W_49E_50I_52G_53A_54D_55F_67L | 3.571955811 |
| 1989 | 4453 | TTCTATTGTAATATCTGTGGGGCACAGTTTactcagaaaggtaacctcctccgccacattaaaCTG | 46F_47Y_49N_50I_52G_53A_54Q_55F_67L | 3.571270542 |
| 1990 | 4233 | TTCTGGTGTAACCAGTGTGGGGCACAATTTactcagaaaggtaacctcctccgccacattaaaCTG | 46F_47W_49N_50Q_52G_53A_54Q_55F_67L | 3.570734129 |
| 1991 | 4394 | TTCTACTGTGAAATCTGTGGGGCAAGATTTactcagaaaggtaacctcctccgccacattaaaCTG | 46F_47Y_49E_50I_52G_53A_54R_55F_67L | 3.569291676 |
| 1992 | 1191 | TTCTTCTGTCCTGTCTGTGGGGCAAACTTTactcagaaaggtaacctcctccgccacattaaaCTG | 46F_47F_49P_50V_52G_53A_54N_55F_67L | 3.567574561 |
| 1993 | 780 | TTCGAGTGTGAGATCTGTGGGGCAGCGTTTactcagaaaggtaacctcctccgccacattaaaCTG | 46F_47E_49E_50I_52G_53A_54A_55F_67L | 3.567522113 |
| 1994 | 4455 | TTCTATTGTAACATCTGTGGGGCATCCTTTactcagaaaggtaacctcctccgccacattaaaCTG | 46F_47Y_49N_50I_52G_53A_54S_55F_67L | 3.559303464 |
| 1995 | 6807 | CTTTGGTGTCCTATATGCGGCTTTATTTGCactcagaaaggtaacctcctccgccacattaaaCTG | 46L_47W_49P_50I_52G_53F_54I_55C_67L | 3.55253085 |
| 1996 | 4317 | TTCTGGTGTCCCGTCTGTGGGGCAGTTTTTactcagaaaggtaacctcctccgccacattaaaCTG | 46F_47W_49P_50V_52G_53A_54V_55F_67L | 3.552408226 |
| 1997 | 1015 | TTCTTTTGTGATGTGTGTGGGGCATCCTTTactcagaaaggtaacctcctccgccacattaaaCTG | 46F_47F_49D_50V_52G_53A_54S_55F_67L | 3.549651058 |
| 1998 | 1072 | TTCTTCTGTGAGGTGTGTGGGGCACCCTTTactcagaaaggtaacctcctccgccacattaaaCTG | 46F_47F_49E_50V_52G_53A_54P_55F_67L | 3.548974015 |
| 1999 | 6541 | CTGTTCTGCCCCATATGTGGGTTCTGTTGTactcagaaaggtaacctcctccgccacattaaaCTG | 46L_47F_49P_50I_52G_53F_54C_55C_67L | 3.547567732 |
| 2000 | 4389 | TTCTACTGTGAAATCTGTGGGGCACTGTTTactcagaaaggtaacctcctccgccacattaaaCTG | 46F_47Y_49E_50I_52G_53A_54L_55F_67L | 3.547193337 |
| 2001 | 976 | TTCTTCTGTGACATTTGTGGGGCAACCTTTactcagaaaggtaacctcctccgccacattaaaCTG | 46F_47F_49D_50I_52G_53A_54T_55F_67L | 3.546100257 |
| 2002 | 4087 | TTCTGGTGTGACATTTGTGGGGCAATCTTTactcagaaaggtaacctcctccgccacattaaaCTG | 46F_47W_49D_50I_52G_53A_54I_55F_67L | 3.546013607 |
| 2003 | 4441 | TTCTATTGTAACATATGTGGGGCATGTTTTactcagaaaggtaacctcctccgccacattaaaCTG | 46F_47Y_49N_50I_52G_53A_54C_55F_67L | 3.544697293 |
| 2004 | 7946 | CTGTGGTGCGAAATTTGCGGGTTTCATTGCactcagaaaggtaacctcctccgccacattaaaCTG | 46L_47W_49E_50I_52G_53F_54H_55C_67L | 3.541503282 |
| 2005 | 4092 | TTCTGGTGTGATATCTGTGGGGCACCCTTTactcagaaaggtaacctcctccgccacattaaaCTG | 46F_47W_49D_50I_52G_53A_54P_55F_67L | 3.537570074 |
| 2006 | 1033 | TTCTTCTGTGAGATCTGTGGGGCACAGTTTactcagaaaggtaacctcctccgccacattaaaCTG | 46F_47F_49E_50I_52G_53A_54Q_55F_67L | 3.534672564 |
| 2007 | 1151 | TTCTTCTGTCCCATTTGTGGGGCAAATTTTactcagaaaggtaacctcctccgccacattaaaCTG | 46F_47F_49P_50I_52G_53A_54N_55F_67L | 3.533840574 |
| 2008 | 4385 | TTCTATTGTGAGATATGTGGGGCAGGGTTTactcagaaaggtaacctcctccgccacattaaaCTG | 46F_47Y_49E_50I_52G_53A_54G_55F_67L | 3.531161729 |
| 2009 | 1029 | TTCTTCTGTGAAATTTGTGGGGCACTGTTTactcagaaaggtaacctcctccgccacattaaaCTG | 46F_47F_49E_50I_52G_53A_54L_55F_67L | 3.530640428 |
| 2010 | 540 | TTCGATTGTGAGATCTGTGGGGCAGCATTTactcagaaaggtaacctcctccgccacattaaaCTG | 46F_47D_49E_50I_52G_53A_54A_55F_67L | 3.524882964 |
| 2011 | 3192 | TTCCGCTGTGAAATCTGTGGGGCACCCTTTactcagaaaggtaacctcctccgccacattaaaCTG | 46F_47R_49E_50I_52G_53A_54P_55F_67L | 3.524354871 |
| 2012 | 1555 | TTCCACTGTGAGGTGTGTGGGGCATCTTTTactcagaaaggtaacctcctccgccacattaaaCTG | 46F_47H_49E_50V_52G_53A_54S_55F_67L | 3.520298853 |
| 2013 | 4432 | TTCTACTGTGAAGTATGTGGGGCACCATTTactcagaaaggtaacctcctccgccacattaaaCTG | 46F_47Y_49E_50V_52G_53A_54P_55F_67L | 3.518969297 |
| 2014 | 6821 | TTGTATTGTCCCATTTGTGGCTTTTGTTGTactcagaaaggtaacctcctccgccacattaaaCTG | 46L_47Y_49P_50I_52G_53F_54C_55C_67L | 3.510227575 |
| 2015 | 4152 | TTCTGGTGTGAAATTTGTGGGGCACCATTTactcagaaaggtaacctcctccgccacattaaaCTG | 46F_47W_49E_50I_52G_53A_54P_55F_67L | 3.508242362 |
| 2016 | 3912 | TTCGTGTGTGAAATCTGTGGGGCACCCTTTactcagaaaggtaacctcctccgccacattaaaCTG | 46F_47V_49E_50I_52G_53A_54P_55F_67L | 3.500044523 |
| 2017 | 4280 | TTCTGGTGTCCCCAGTGTGGGGCAGCTTTTactcagaaaggtaacctcctccgccacattaaaCTG | 46F_47W_49P_50Q_52G_53A_54A_55F_67L | 3.49979629 |
| 2018 | 4541 | TTCTACTGTCCGGTTTGTGGGGCATGCTTTactcagaaaggtaacctcctccgccacattaaaCTG | 46F_47Y_49P_50V_52G_53A_54C_55F_67L | 3.495660239 |
| 2019 | 1632 | TTCCACTGTCCAATTTGTGGGGCACCTTTTactcagaaaggtaacctcctccgccacattaaaCTG | 46F_47H_49P_50I_52G_53A_54P_55F_67L | 3.493253795 |
| 2020 | 4266 | TTCTGGTGTCCGATCTGTGGGGCACATTTTactcagaaaggtaacctcctccgccacattaaaCTG | 46F_47W_49P_50I_52G_53A_54H_55F_67L | 3.490899591 |
| 2021 | 595 | TTCGATTGTGAAGTCTGTGGGGCATCTTTTactcagaaaggtaacctcctccgccacattaaaCTG | 46F_47D_49E_50V_52G_53A_54S_55F_67L | 3.487326631 |
| 2022 | 4189 | TTCTGGTGTGAAGTTTGTGGGGCACTCTTTactcagaaaggtaacctcctccgccacattaaaCTG | 46F_47W_49E_50V_52G_53A_54L_55F_67L | 3.487204124 |
| 2023 | 1180 | TTCTTCTGTCCCGTGTGTGGGGCAGCATTTactcagaaaggtaacctcctccgccacattaaaCTG | 46F_47F_49P_50V_52G_53A_54A_55F_67L | 3.483109807 |
| 2024 | 4032 | TTCGTGTGTCCTATCTGTGGGGCACCTTTTactcagaaaggtaacctcctccgccacattaaaCTG | 46F_47V_49P_50I_52G_53A_54P_55F_67L | 3.48196446 |
| 2025 | 7680 | TTGTTTTGTGAGATCTGTGGTTTTGCGTGCactcagaaaggtaacctcctccgccacattaaaCTG | 46L_47F_49E_50I_52G_53F_54A_55C_67L | 3.479586038 |
| 2026 | 1027 | TTCTTCTGTGAGATCTGTGGGGCAATTTTTactcagaaaggtaacctcctccgccacattaaaCTG | 46F_47F_49E_50I_52G_53A_54I_55F_67L | 3.47819927 |
| 2027 | 4207 | TTCTGGTGTAATATTTGTGGGGCAATATTTactcagaaaggtaacctcctccgccacattaaaCTG | 46F_47W_49N_50I_52G_53A_54I_55F_67L | 3.477231005 |
| 2028 | 1540 | TTCCATTGTGAAGTGTGTGGGGCAGCCTTTactcagaaaggtaacctcctccgccacattaaaCTG | 46F_47H_49E_50V_52G_53A_54A_55F_67L | 3.476969087 |
| 2029 | 4371 | TTCTATTGTGATGTGTGTGGGGCAAACTTTactcagaaaggtaacctcctccgccacattaaaCTG | 46F_47Y_49D_50V_52G_53A_54N_55F_67L | 3.474659346 |
| 2030 | 4113 | TTCTGGTGTGATCAATGTGGGGCACAGTTTactcagaaaggtaacctcctccgccacattaaaCTG | 46F_47W_49D_50Q_52G_53A_54Q_55F_67L | 3.473290604 |
| 2031 | 6732 | CTGAGATGTCCTATTTGTGGCTTTCCCTGCactcagaaaggtaacctcctccgccacattaaaCTG | 46L_47R_49P_50I_52G_53F_54P_55C_67L | 3.469844391 |
| 2032 | 6826 | CTCTACTGTCCCATCTGTGGCTTCCACTGCactcagaaaggtaacctcctccgccacattaaaCTG | 46L_47Y_49P_50I_52G_53F_54H_55C_67L | 3.463871591 |
| 2033 | 7966 | CTGTACTGCGAGATCTGTGGCTTCCATTGTactcagaaaggtaacctcctccgccacattaaaCTG | 46L_47Y_49E_50I_52G_53F_54H_55C_67L | 3.462249986 |
| 2034 | 4256 | TTCTGGTGTAATGTGTGTGGGGCAACCTTTactcagaaaggtaacctcctccgccacattaaaCTG | 46F_47W_49N_50V_52G_53A_54T_55F_67L | 3.460625995 |
| 2035 | 4376 | TTCTACTGTGACGTTTGTGGGGCAACCTTTactcagaaaggtaacctcctccgccacattaaaCTG | 46F_47Y_49D_50V_52G_53A_54T_55F_67L | 3.458720432 |
| 2036 | 6808 | CTGTGGTGTCCTATCTGTGGGTTTAAGTGTactcagaaaggtaacctcctccgccacattaaaCTG | 46L_47W_49P_50I_52G_53F_54K_55C_67L | 3.453061152 |
| 2037 | 4430 | TTCTATTGTGAAGTTTGTGGGGCAATGTTTactcagaaaggtaacctcctccgccacattaaaCTG | 46F_47Y_49E_50V_52G_53A_54M_55F_67L | 3.451928481 |
| 2038 | 4085 | TTCTGGTGTGACATATGTGGGGCAGGGTTTactcagaaaggtaacctcctccgccacattaaaCTG | 46F_47W_49D_50I_52G_53A_54G_55F_67L | 3.450193154 |
| 2039 | 7948 | CTGTGGTGCGAAATCTGCGGATTCAAGTGTactcagaaaggtaacctcctccgccacattaaaCTG | 46L_47W_49E_50I_52G_53F_54K_55C_67L | 3.450110619 |
| 2040 | 1792 | TTCATCTGTGAGGTGTGTGGGGCACCATTTactcagaaaggtaacctcctccgccacattaaaCTG | 46F_47I_49E_50V_52G_53A_54P_55F_67L | 3.446277315 |
| 2041 | 4204 | TTCTGGTGTAACATATGTGGGGCATTTTTTactcagaaaggtaacctcctccgccacattaaaCTG | 46F_47W_49N_50I_52G_53A_54F_55F_67L | 3.445800416 |
| 2042 | 795 | TTCGAATGTGAGATTTGTGGGGCAAGCTTTactcagaaaggtaacctcctccgccacattaaaCTG | 46F_47E_49E_50I_52G_53A_54S_55F_67L | 3.445766282 |
| 2043 | 6553 | TTATTCTGCCCTATCTGTGGCTTTCAGTGCactcagaaaggtaacctcctccgccacattaaaCTG | 46L_47F_49P_50I_52G_53F_54Q_55C_67L | 3.441102782 |
| 2044 | 1513 | TTCCATTGTGAGATCTGTGGGGCACAATTTactcagaaaggtaacctcctccgccacattaaaCTG | 46F_47H_49E_50I_52G_53A_54Q_55F_67L | 3.440365794 |
| 2045 | 792 | TTCGAATGTGAAATCTGTGGGGCACCCTTTactcagaaaggtaacctcctccgccacattaaaCTG | 46F_47E_49E_50I_52G_53A_54P_55F_67L | 3.438796432 |
| 2046 | 4084 | TTCTGGTGTGATATTTGTGGGGCATTCTTTactcagaaaggtaacctcctccgccacattaaaCTG | 46F_47W_49D_50I_52G_53A_54F_55F_67L | 3.43878597 |
| 2047 | 975 | TTCTTCTGTGACATCTGTGGGGCAAGTTTTactcagaaaggtaacctcctccgccacattaaaCTG | 46F_47F_49D_50I_52G_53A_54S_55F_67L | 3.43563391 |
| 2048 | 7967 | CTCTACTGCGAGATCTGTGGGTTCATATGCactcagaaaggtaacctcctccgccacattaaaCTG | 46L_47Y_49E_50I_52G_53F_54I_55C_67L | 3.434813922 |
| 2049 | 4501 | TTCTATTGTCCTATCTGTGGGGCATGCTTTactcagaaaggtaacctcctccgccacattaaaCTG | 46F_47Y_49P_50I_52G_53A_54C_55F_67L | 3.43356469 |
| 2050 | 4308 | TTCTGGTGTCCAGTGTGTGGGGCAAAGTTTactcagaaaggtaacctcctccgccacattaaaCTG | 46F_47W_49P_50V_52G_53A_54K_55F_67L | 3.429650007 |
| 2051 | 4540 | TTCTACTGTCCAGTCTGTGGGGCAGCTTTTactcagaaaggtaacctcctccgccacattaaaCTG | 46F_47Y_49P_50V_52G_53A_54A_55F_67L | 3.426798383 |
| 2052 | 4303 | TTCTGGTGTCCAGTCTGTGGGGCAGAATTTactcagaaaggtaacctcctccgccacattaaaCTG | 46F_47W_49P_50V_52G_53A_54E_55F_67L | 3.426673639 |
| 2053 | 4555 | TTCTACTGTCCCGTGTGTGGGGCAAGCTTTactcagaaaggtaacctcctccgccacattaaaCTG | 46F_47Y_49P_50V_52G_53A_54S_55F_67L | 3.422679829 |
| 2054 | 1512 | TTCCATTGTGAGATCTGTGGGGCACCATTTactcagaaaggtaacctcctccgccacattaaaCTG | 46F_47H_49E_50I_52G_53A_54P_55F_67L | 3.421022425 |
| 2055 | 3420 | TTCAGCTGTGAGATATGTGGGGCAGCGTTTactcagaaaggtaacctcctccgccacattaaaCTG | 46F_47S_49E_50I_52G_53A_54A_55F_67L | 3.420213413 |
| 2056 | 4127 | TTCTGGTGTGACGTGTGTGGGGCAATATTTactcagaaaggtaacctcctccgccacattaaaCTG | 46F_47W_49D_50V_52G_53A_54I_55F_67L | 3.417674852 |
| 2057 | 1011 | TTCTTTTGTGATGTTTGTGGGGCAAACTTTactcagaaaggtaacctcctccgccacattaaaCTG | 46F_47F_49D_50V_52G_53A_54N_55F_67L | 3.413674056 |
| 2058 | 1195 | TTCTTCTGTCCCGTTTGTGGGGCATCCTTTactcagaaaggtaacctcctccgccacattaaaCTG | 46F_47F_49P_50V_52G_53A_54S_55F_67L | 3.408499139 |
| 2059 | 4263 | TTCTGGTGTCCTATTTGTGGGGCAGAGTTTactcagaaaggtaacctcctccgccacattaaaCTG | 46F_47W_49P_50I_52G_53A_54E_55F_67L | 3.408301052 |
| 2060 | 960 | TTCTTCTGTGACATCTGTGGGGCAGCCTTTactcagaaaggtaacctcctccgccacattaaaCTG | 46F_47F_49D_50I_52G_53A_54A_55F_67L | 3.406345116 |
| 2061 | 6804 | CTATGGTGTCCAATCTGCGGCTTTTTTTGCactcagaaaggtaacctcctccgccacattaaaCTG | 46L_47W_49P_50I_52G_53F_54F_55C_67L | 3.403835656 |
| 2062 | 3072 | TTCCAATGTCCTATTTGTGGGGCACCATTTactcagaaaggtaacctcctccgccacattaaaCTG | 46F_47Q_49P_50I_52G_53A_54P_55F_67L | 3.402505581 |
| 2063 | 4083 | TTCTGGTGTGATATTTGTGGGGCAGAATTTactcagaaaggtaacctcctccgccacattaaaCTG | 46F_47W_49D_50I_52G_53A_54E_55F_67L | 3.40108435 |
| 2064 | 4326 | TTCTATTGTGATATTTGTGGGGCACATTTTactcagaaaggtaacctcctccgccacattaaaCTG | 46F_47Y_49D_50I_52G_53A_54H_55F_67L | 3.390988389 |
| 2065 | 3852 | TTCGTCTGTGATATTTGTGGGGCACCATTTactcagaaaggtaacctcctccgccacattaaaCTG | 46F_47V_49D_50I_52G_53A_54P_55F_67L | 3.390466932 |
| 2066 | 1872 | TTCATCTGTCCTATTTGTGGGGCACCTTTTactcagaaaggtaacctcctccgccacattaaaCTG | 46F_47I_49P_50I_52G_53A_54P_55F_67L | 3.386400536 |
| 2067 | 4503 | TTCTACTGTCCGATTTGTGGGGCAGAATTTactcagaaaggtaacctcctccgccacattaaaCTG | 46F_47Y_49P_50I_52G_53A_54E_55F_67L | 3.384516818 |
| 2068 | 7947 | TTGTGGTGTGAAATCTGCGGCTTCATTTGCactcagaaaggtaacctcctccgccacattaaaCTG | 46L_47W_49E_50I_52G_53F_54I_55C_67L | 3.381974458 |
| 2069 | 7681 | CTGTTTTGTGAAATTTGTGGATTTTGTTGTactcagaaaggtaacctcctccgccacattaaaCTG | 46L_47F_49E_50I_52G_53F_54C_55C_67L | 3.38147161 |
| 2070 | 4416 | TTCTACTGTGAACAGTGTGGGGCAACCTTTactcagaaaggtaacctcctccgccacattaaaCTG | 46F_47Y_49E_50Q_52G_53A_54T_55F_67L | 3.379446724 |
| 2071 | 1030 | TTCTTTTGTGAGATCTGTGGGGCAATGTTTactcagaaaggtaacctcctccgccacattaaaCTG | 46F_47F_49E_50I_52G_53A_54M_55F_67L | 3.379358649 |
| 2072 | 4100 | TTCTGGTGTGATCAGTGTGGGGCAGCCTTTactcagaaaggtaacctcctccgccacattaaaCTG | 46F_47W_49D_50Q_52G_53A_54A_55F_67L | 3.374982029 |
| 2073 | 4254 | TTCTGGTGTAACGTCTGTGGGGCACGTTTTactcagaaaggtaacctcctccgccacattaaaCTG | 46F_47W_49N_50V_52G_53A_54R_55F_67L | 3.374176961 |
| 2074 | 971 | TTCTTTTGTGATATCTGTGGGGCAAATTTTactcagaaaggtaacctcctccgccacattaaaCTG | 46F_47F_49D_50I_52G_53A_54N_55F_67L | 3.369819308 |
| 2075 | 1500 | TTCCACTGTGAAATATGTGGGGCAGCGTTTactcagaaaggtaacctcctccgccacattaaaCTG | 46F_47H_49E_50I_52G_53A_54A_55F_67L | 3.367589575 |
| 2076 | 4452 | TTCTACTGTAATATTTGTGGGGCACCATTTactcagaaaggtaacctcctccgccacattaaaCTG | 46F_47Y_49N_50I_52G_53A_54P_55F_67L | 3.366912808 |
| 2077 | 3460 | TTCAGCTGTGAGGTCTGTGGGGCAGCTTTTactcagaaaggtaacctcctccgccacattaaaCTG | 46F_47S_49E_50V_52G_53A_54A_55F_67L | 3.36528858 |
| 2078 | 4265 | TTCTGGTGTCCAATCTGTGGGGCAGGTTTTactcagaaaggtaacctcctccgccacattaaaCTG | 46F_47W_49P_50I_52G_53A_54G_55F_67L | 3.357597226 |
| 2079 | 4250 | TTCTGGTGTAATGTCTGTGGGGCAATGTTTactcagaaaggtaacctcctccgccacattaaaCTG | 46F_47W_49N_50V_52G_53A_54M_55F_67L | 3.349291342 |
| 2080 | 1912 | TTCATCTGTCCAGTCTGTGGGGCACCCTTTactcagaaaggtaacctcctccgccacattaaaCTG | 46F_47I_49P_50V_52G_53A_54P_55F_67L | 3.34118659 |
| 2081 | 4399 | TTCTACTGTGAGATCTGTGGGGCATACTTTactcagaaaggtaacctcctccgccacattaaaCTG | 46F_47Y_49E_50I_52G_53A_54Y_55F_67L | 3.340998145 |
| 2082 | 1181 | TTCTTCTGTCCAGTATGTGGGGCATGCTTTactcagaaaggtaacctcctccgccacattaaaCTG | 46F_47F_49P_50V_52G_53A_54C_55F_67L | 3.335874186 |
| 2083 | 1000 | TTCTTTTGTGACGTGTGTGGGGCAGCGTTTactcagaaaggtaacctcctccgccacattaaaCTG | 46F_47F_49D_50V_52G_53A_54A_55F_67L | 3.335439996 |
| 2084 | 4552 | TTCTATTGTCCAGTGTGTGGGGCACCTTTTactcagaaaggtaacctcctccgccacattaaaCTG | 46F_47Y_49P_50V_52G_53A_54P_55F_67L | 3.334057541 |
| 2085 | 4515 | TTCTACTGTCCGATCTGTGGGGCAAGTTTTactcagaaaggtaacctcctccgccacattaaaCTG | 46F_47Y_49P_50I_52G_53A_54S_55F_67L | 3.333039878 |
| 2086 | 1092 | TTCTTCTGTAACATCTGTGGGGCACCTTTTactcagaaaggtaacctcctccgccacattaaaCTG | 46F_47F_49N_50I_52G_53A_54P_55F_67L | 3.330411259 |
| 2087 | 6557 | CTGTTCTGTCCGATCTGTGGCTTCGTATGTactcagaaaggtaacctcctccgccacattaaaCTG | 46L_47F_49P_50I_52G_53F_54V_55C_67L | 3.325999957 |
| 2088 | 1067 | TTCTTCTGTGAGGTATGTGGGGCAATATTTactcagaaaggtaacctcctccgccacattaaaCTG | 46F_47F_49E_50V_52G_53A_54I_55F_67L | 3.324176126 |
| 2089 | 4439 | TTCTACTGTGAAGTGTGTGGGGCATACTTTactcagaaaggtaacctcctccgccacattaaaCTG | 46F_47Y_49E_50V_52G_53A_54Y_55F_67L | 3.322171962 |
| 2090 | 7974 | CTGTATTGTGAAATTTGCGGCTTTCGCTGCactcagaaaggtaacctcctccgccacattaaaCTG | 46L_47Y_49E_50I_52G_53F_54R_55C_67L | 3.319332458 |
| 2091 | 4139 | TTCTGGTGTGACGTGTGTGGGGCATACTTTactcagaaaggtaacctcctccgccacattaaaCTG | 46F_47W_49D_50V_52G_53A_54Y_55F_67L | 3.317928197 |
| 2092 | 6803 | TTGTGGTGTCCTATTTGTGGGTTCGAGTGCactcagaaaggtaacctcctccgccacattaaaCTG | 46L_47W_49P_50I_52G_53F_54E_55C_67L | 3.316912513 |
| 2093 | 4425 | TTCTACTGTGAGGTGTGTGGGGCAGGATTTactcagaaaggtaacctcctccgccacattaaaCTG | 46F_47Y_49E_50V_52G_53A_54G_55F_67L | 3.315137199 |
| 2094 | 4512 | TTCTATTGTCCAATATGTGGGGCACCGTTTactcagaaaggtaacctcctccgccacattaaaCTG | 46F_47Y_49P_50I_52G_53A_54P_55F_67L | 3.3108082 |
| 2095 | 7970 | CTCTATTGTGAGATCTGTGGATTTATGTGTactcagaaaggtaacctcctccgccacattaaaCTG | 46L_47Y_49E_50I_52G_53F_54M_55C_67L | 3.309637578 |
| 2096 | 4220 | TTCTGGTGTAATCAGTGTGGGGCAGCATTTactcagaaaggtaacctcctccgccacattaaaCTG | 46F_47W_49N_50Q_52G_53A_54A_55F_67L | 3.308902063 |
| 2097 | 1152 | TTCTTCTGTCCAATTTGTGGGGCACCATTTactcagaaaggtaacctcctccgccacattaaaCTG | 46F_47F_49P_50I_52G_53A_54P_55F_67L | 3.305299231 |
| 2098 | 1039 | TTCTTTTGTGAAATATGTGGGGCATATTTTactcagaaaggtaacctcctccgccacattaaaCTG | 46F_47F_49E_50I_52G_53A_54Y_55F_67L | 3.305287903 |
| 2099 | 6540 | CTGTTCTGCCCGATCTGCGGTTTCGCCTGCactcagaaaggtaacctcctccgccacattaaaCTG | 46L_47F_49P_50I_52G_53F_54A_55C_67L | 3.304196095 |
| 2100 | 4270 | TTCTGGTGTCCAATTTGTGGGGCAATGTTTactcagaaaggtaacctcctccgccacattaaaCTG | 46F_47W_49P_50I_52G_53A_54M_55F_67L | 3.301672447 |
| 2101 | 6819 | CTGTGGTGTCCCATCTGCGGCTTTTATTGCactcagaaaggtaacctcctccgccacattaaaCTG | 46L_47W_49P_50I_52G_53F_54Y_55C_67L | 3.300697589 |
| 2102 | 6820 | CTCTACTGTCCGATCTGTGGCTTTGCTTGTactcagaaaggtaacctcctccgccacattaaaCTG | 46L_47Y_49P_50I_52G_53F_54A_55C_67L | 3.300356383 |
| 2103 | 4281 | TTCTGGTGTCCCCAGTGTGGGGCATGCTTTactcagaaaggtaacctcctccgccacattaaaCTG | 46F_47W_49P_50Q_52G_53A_54C_55F_67L | 3.29899001 |
| 2104 | 7690 | TTATTCTGTGAGATCTGTGGATTTATGTGCactcagaaaggtaacctcctccgccacattaaaCTG | 46L_47F_49E_50I_52G_53F_54M_55C_67L | 3.293799976 |
| 2105 | 1157 | TTCTTCTGTCCCATCTGTGGGGCAGTCTTTactcagaaaggtaacctcctccgccacattaaaCTG | 46F_47F_49P_50I_52G_53A_54V_55F_67L | 3.29308605 |
| 2106 | 1140 | TTCTTCTGTCCAATTTGTGGGGCAGCCTTTactcagaaaggtaacctcctccgccacattaaaCTG | 46F_47F_49P_50I_52G_53A_54A_55F_67L | 3.285833231 |
| 2107 | 4506 | TTCTACTGTCCGATCTGTGGGGCACATTTTactcagaaaggtaacctcctccgccacattaaaCTG | 46F_47Y_49P_50I_52G_53A_54H_55F_67L | 3.284486559 |
| 2108 | 3952 | TTCGTGTGTGAGGTGTGTGGGGCACCTTTTactcagaaaggtaacctcctccgccacattaaaCTG | 46F_47V_49E_50V_52G_53A_54P_55F_67L | 3.283371982 |
| 2109 | 4203 | TTCTGGTGTAACATTTGTGGGGCAGAATTTactcagaaaggtaacctcctccgccacattaaaCTG | 46F_47W_49N_50I_52G_53A_54E_55F_67L | 3.282850521 |
| 2110 | 4248 | TTCTGGTGTAACGTGTGTGGGGCAAAGTTTactcagaaaggtaacctcctccgccacattaaaCTG | 46F_47W_49N_50V_52G_53A_54K_55F_67L | 3.281594286 |
| 2111 | 7959 | TTGTGGTGCGAGATCTGCGGCTTCTATTGCactcagaaaggtaacctcctccgccacattaaaCTG | 46L_47W_49E_50I_52G_53F_54Y_55C_67L | 3.281481919 |
| 2112 | 4101 | TTCTGGTGTGACCAATGTGGGGCATGCTTTactcagaaaggtaacctcctccgccacattaaaCTG | 46F_47W_49D_50Q_52G_53A_54C_55F_67L | 3.28099575 |
| 2113 | 4176 | TTCTGGTGTGAACAATGTGGGGCAACATTTactcagaaaggtaacctcctccgccacattaaaCTG | 46F_47W_49E_50Q_52G_53A_54T_55F_67L | 3.280809402 |
| 2114 | 6830 | TTATATTGTCCTATCTGTGGATTCATGTGCactcagaaaggtaacctcctccgccacattaaaCTG | 46L_47Y_49P_50I_52G_53F_54M_55C_67L | 3.280677813 |
| 2115 | 3672 | TTCACGTGTGAGATCTGTGGGGCACCCTTTactcagaaaggtaacctcctccgccacattaaaCTG | 46F_47T_49E_50I_52G_53A_54P_55F_67L | 3.279694583 |
| 2116 | 4319 | TTCTGGTGTCCTGTTTGTGGGGCATACTTTactcagaaaggtaacctcctccgccacattaaaCTG | 46F_47W_49P_50V_52G_53A_54Y_55F_67L | 3.27865792 |
| 2117 | 4123 | TTCTGGTGTGATGTGTGTGGGGCAGAGTTTactcagaaaggtaacctcctccgccacattaaaCTG | 46F_47W_49D_50V_52G_53A_54E_55F_67L | 3.277524224 |
| 2118 | 1143 | TTCTTCTGTCCAATTTGTGGGGCAGAGTTTactcagaaaggtaacctcctccgccacattaaaCTG | 46F_47F_49P_50I_52G_53A_54E_55F_67L | 3.274031123 |
| 2119 | 4205 | TTCTGGTGTAATATATGTGGGGCAGGTTTTactcagaaaggtaacctcctccgccacattaaaCTG | 46F_47W_49N_50I_52G_53A_54G_55F_67L | 3.271528641 |
| 2120 | 1068 | TTCTTTTGTGAGGTGTGTGGGGCAAAATTTactcagaaaggtaacctcctccgccacattaaaCTG | 46F_47F_49E_50V_52G_53A_54K_55F_67L | 3.265606842 |
| 2121 | 1074 | TTCTTTTGTGAGGTATGTGGGGCAAGGTTTactcagaaaggtaacctcctccgccacattaaaCTG | 46F_47F_49E_50V_52G_53A_54R_55F_67L | 3.261577394 |
| 2122 | 1183 | TTCTTCTGTCCGGTGTGTGGGGCAGAATTTactcagaaaggtaacctcctccgccacattaaaCTG | 46F_47F_49P_50V_52G_53A_54E_55F_67L | 3.256613877 |
| 2123 | 4451 | TTCTACTGTAATATTTGTGGGGCAAACTTTactcagaaaggtaacctcctccgccacattaaaCTG | 46F_47Y_49N_50I_52G_53A_54N_55F_67L | 3.252723823 |
| 2124 | 3475 | TTCAGCTGTGAAGTGTGTGGGGCAAGTTTTactcagaaaggtaacctcctccgccacattaaaCTG | 46F_47S_49E_50V_52G_53A_54S_55F_67L | 3.25004452 |
| 2125 | 972 | TTCTTCTGTGACATATGTGGGGCACCCTTTactcagaaaggtaacctcctccgccacattaaaCTG | 46F_47F_49D_50I_52G_53A_54P_55F_67L | 3.247063534 |
| 2126 | 300 | TTCTGCTGTGAAATCTGTGGGGCAGCCTTTactcagaaaggtaacctcctccgccacattaaaCTG | 46F_47C_49E_50I_52G_53A_54A_55F_67L | 3.244024104 |
| 2127 | 6839 | CTCTATTGTCCAATCTGCGGTTTTTATTGCactcagaaaggtaacctcctccgccacattaaaCTG | 46L_47Y_49P_50I_52G_53F_54Y_55C_67L | 3.241320563 |
| 2128 | 4361 | TTCTACTGTGACGTTTGTGGGGCATGTTTTactcagaaaggtaacctcctccgccacattaaaCTG | 46F_47Y_49D_50V_52G_53A_54C_55F_67L | 3.239538169 |
| 2129 | 4456 | TTCTATTGTAACATCTGTGGGGCAACATTTactcagaaaggtaacctcctccgccacattaaaCTG | 46F_47Y_49N_50I_52G_53A_54T_55F_67L | 3.239508101 |
| 2130 | 4312 | TTCTGGTGTCCTGTGTGTGGGGCACCATTTactcagaaaggtaacctcctccgccacattaaaCTG | 46F_47W_49P_50V_52G_53A_54P_55F_67L | 3.238387225 |
| 2131 | 1022 | TTCTTCTGTGAAATCTGTGGGGCAGACTTTactcagaaaggtaacctcctccgccacattaaaCTG | 46F_47F_49E_50I_52G_53A_54D_55F_67L | 3.234910287 |
| 2132 | 6595 | TTACATTGCCCAATATGTGGCTTCTCCTGTactcagaaaggtaacctcctccgccacattaaaCTG | 46L_47H_49P_50I_52G_53F_54S_55C_67L | 3.231603133 |
| 2133 | 553 | TTCGACTGTGAGATATGTGGGGCACAGTTTactcagaaaggtaacctcctccgccacattaaaCTG | 46F_47D_49E_50I_52G_53A_54Q_55F_67L | 3.230785829 |
| 2134 | 3660 | TTCACGTGTGAAATTTGTGGGGCAGCTTTTactcagaaaggtaacctcctccgccacattaaaCTG | 46F_47T_49E_50I_52G_53A_54A_55F_67L | 3.221004533 |
| 2135 | 3132 | TTCAGGTGTGATATCTGTGGGGCACCCTTTactcagaaaggtaacctcctccgccacattaaaCTG | 46F_47R_49D_50I_52G_53A_54P_55F_67L | 3.220237272 |
| 2136 | 1016 | TTCTTCTGTGATGTTTGTGGGGCAACTTTTactcagaaaggtaacctcctccgccacattaaaCTG | 46F_47F_49D_50V_52G_53A_54T_55F_67L | 3.217983946 |
| 2137 | 554 | TTCGATTGTGAGATTTGTGGGGCACGGTTTactcagaaaggtaacctcctccgccacattaaaCTG | 46F_47D_49E_50I_52G_53A_54R_55F_67L | 3.217629929 |
| 2138 | 4221 | TTCTGGTGTAATCAGTGTGGGGCATGCTTTactcagaaaggtaacctcctccgccacattaaaCTG | 46F_47W_49N_50Q_52G_53A_54C_55F_67L | 3.217304845 |
| 2139 | 1141 | TTCTTTTGTCCGATTTGTGGGGCATGCTTTactcagaaaggtaacctcctccgccacattaaaCTG | 46F_47F_49P_50I_52G_53A_54C_55F_67L | 3.21585266 |
| 2140 | 1032 | TTCTTTTGTGAAATCTGTGGGGCACCCTTTactcagaaaggtaacctcctccgccacattaaaCTG | 46F_47F_49E_50I_52G_53A_54P_55F_67L | 3.21455038 |
| 2141 | 7968 | TTATACTGCGAGATATGCGGCTTCAAATGCactcagaaaggtaacctcctccgccacattaaaCTG | 46L_47Y_49E_50I_52G_53F_54K_55C_67L | 3.2109954 |
| 2142 | 7573 | TTCTGGTGTGAAATTTGTGGCCGCCAGTTTactcagaaaggtaacctcctccgccacattaaaCTG | 46F_47W_49E_50I_52G_53R_54Q_55F_67L | 3.205332236 |
| 2143 | 2592 | TTCATGTGTCCCATATGTGGGGCACCCTTTactcagaaaggtaacctcctccgccacattaaaCTG | 46F_47M_49P_50I_52G_53A_54P_55F_67L | 3.204435467 |
| 2144 | 1692 | TTCATATGTGATATCTGTGGGGCACCATTTactcagaaaggtaacctcctccgccacattaaaCTG | 46F_47I_49D_50I_52G_53A_54P_55F_67L | 3.202820304 |
| 2145 | 4491 | TTCTATTGTAACGTTTGTGGGGCAAACTTTactcagaaaggtaacctcctccgccacattaaaCTG | 46F_47Y_49N_50V_52G_53A_54N_55F_67L | 3.202405981 |
| 2146 | 2992 | TTCCAATGTGAGGTCTGTGGGGCACCATTTactcagaaaggtaacctcctccgccacattaaaCTG | 46F_47Q_49E_50V_52G_53A_54P_55F_67L | 3.201854295 |
| 2147 | 4328 | TTCTACTGTGATATTTGTGGGGCAAAGTTTactcagaaaggtaacctcctccgccacattaaaCTG | 46F_47Y_49D_50I_52G_53A_54K_55F_67L | 3.200168302 |
| 2148 | 4519 | TTCTACTGTCCAATATGTGGGGCATACTTTactcagaaaggtaacctcctccgccacattaaaCTG | 46F_47Y_49P_50I_52G_53A_54Y_55F_67L | 3.1995754 |
| 2149 | 6543 | CTGTTCTGCCCCATCTGCGGATTCGAGTGTactcagaaaggtaacctcctccgccacattaaaCTG | 46L_47F_49P_50I_52G_53F_54E_55C_67L | 3.195930558 |
| 2150 | 7979 | CTGTATTGTGAGATTTGCGGTTTCTACTGTactcagaaaggtaacctcctccgccacattaaaCTG | 46L_47Y_49E_50I_52G_53F_54Y_55C_67L | 3.195012015 |
| 2151 | 7943 | CTGTGGTGTGAAATCTGCGGCTTCGAGTGTactcagaaaggtaacctcctccgccacattaaaCTG | 46L_47W_49E_50I_52G_53F_54E_55C_67L | 3.192728833 |
| 2152 | 4546 | TTCTATTGTCCTGTGTGTGGGGCACACTTTactcagaaaggtaacctcctccgccacattaaaCTG | 46F_47Y_49P_50V_52G_53A_54H_55F_67L | 3.190500835 |
| 2153 | 3112 | TTCCAATGTCCCGTTTGTGGGGCACCTTTTactcagaaaggtaacctcctccgccacattaaaCTG | 46F_47Q_49P_50V_52G_53A_54P_55F_67L | 3.183922542 |
| 2154 | 7963 | TTGTATTGCGAAATTTGCGGCTTTGAGTGCactcagaaaggtaacctcctccgccacattaaaCTG | 46L_47Y_49E_50I_52G_53F_54E_55C_67L | 3.183741932 |
| 2155 | 6433 | TTTTGGTGTCCAATATGCGGCCGGCAGTTTactcagaaaggtaacctcctccgccacattaaaCTG | 46F_47W_49P_50I_52G_53R_54Q_55F_67L | 3.18283531 |
| 2156 | 6823 | TTGTATTGTCCGATCTGCGGGTTCGAGTGCactcagaaaggtaacctcctccgccacattaaaCTG | 46L_47Y_49P_50I_52G_53F_54E_55C_67L | 3.182656197 |
| 2157 | 963 | TTCTTTTGTGACATATGTGGGGCAGAGTTTactcagaaaggtaacctcctccgccacattaaaCTG | 46F_47F_49D_50I_52G_53A_54E_55F_67L | 3.182488166 |
| 2158 | 4174 | TTCTGGTGTGAACAGTGTGGGGCACGCTTTactcagaaaggtaacctcctccgccacattaaaCTG | 46F_47W_49E_50Q_52G_53A_54R_55F_67L | 3.182149931 |
| 2159 | 1066 | TTCTTCTGTGAGGTTTGTGGGGCACACTTTactcagaaaggtaacctcctccgccacattaaaCTG | 46F_47F_49E_50V_52G_53A_54H_55F_67L | 3.178454383 |
| 2160 | 60 | TTCGCATGTGAGATCTGTGGGGCAGCCTTTactcagaaaggtaacctcctccgccacattaaaCTG | 46F_47A_49E_50I_52G_53A_54A_55F_67L | 3.177995177 |
| 2161 | 3712 | TTCACATGTGAGGTATGTGGGGCACCTTTTactcagaaaggtaacctcctccgccacattaaaCTG | 46F_47T_49E_50V_52G_53A_54P_55F_67L | 3.177648302 |
| 2162 | 4267 | TTCTGGTGTCCCATTTGTGGGGCAATCTTTactcagaaaggtaacctcctccgccacattaaaCTG | 46F_47W_49P_50I_52G_53A_54I_55F_67L | 3.177409029 |
| 2163 | 4401 | TTCTATTGTGAACAGTGTGGGGCATGTTTTactcagaaaggtaacctcctccgccacattaaaCTG | 46F_47Y_49E_50Q_52G_53A_54C_55F_67L | 3.169550646 |
| 2164 | 4192 | TTCTGGTGTGAGGTGTGTGGGGCACCCTTTactcagaaaggtaacctcctccgccacattaaaCTG | 46F_47W_49E_50V_52G_53A_54P_55F_67L | 3.16692262 |
| 2165 | 4323 | TTCTACTGTGACATTTGTGGGGCAGAGTTTactcagaaaggtaacctcctccgccacattaaaCTG | 46F_47Y_49D_50I_52G_53A_54E_55F_67L | 3.165343499 |
| 2166 | 6827 | TTATACTGTCCAATTTGTGGTTTTATTTGCactcagaaaggtaacctcctccgccacattaaaCTG | 46L_47Y_49P_50I_52G_53F_54I_55C_67L | 3.162689118 |
| 2167 | 4366 | TTCTATTGTGATGTTTGTGGGGCACATTTTactcagaaaggtaacctcctccgccacattaaaCTG | 46F_47Y_49D_50V_52G_53A_54H_55F_67L | 3.162377754 |
| 2168 | 1025 | TTCTTCTGTGAAATTTGTGGGGCAGGGTTTactcagaaaggtaacctcctccgccacattaaaCTG | 46F_47F_49E_50I_52G_53A_54G_55F_67L | 3.157410587 |
| 2169 | 340 | TTCTGTTGTGAAGTGTGTGGGGCAGCCTTTactcagaaaggtaacctcctccgccacattaaaCTG | 46F_47C_49E_50V_52G_53A_54A_55F_67L | 3.156455647 |
| 2170 | 4310 | TTCTGGTGTCCAGTCTGTGGGGCAATGTTTactcagaaaggtaacctcctccgccacattaaaCTG | 46F_47W_49P_50V_52G_53A_54M_55F_67L | 3.156434803 |
| 2171 | 835 | TTCGAATGTGAAGTGTGTGGGGCATCTTTTactcagaaaggtaacctcctccgccacattaaaCTG | 46F_47E_49E_50V_52G_53A_54S_55F_67L | 3.151045199 |
| 2172 | 7951 | CTGTGGTGTGAGATTTGTGGATTCAATTGCactcagaaaggtaacctcctccgccacattaaaCTG | 46L_47W_49E_50I_52G_53F_54N_55C_67L | 3.150867788 |
| 2173 | 4457 | TTCTATTGTAACATCTGTGGGGCAGTCTTTactcagaaaggtaacctcctccgccacattaaaCTG | 46F_47Y_49N_50I_52G_53A_54V_55F_67L | 3.150495376 |
| 2174 | 1452 | TTCCATTGTGACATCTGTGGGGCACCGTTTactcagaaaggtaacctcctccgccacattaaaCTG | 46F_47H_49D_50I_52G_53A_54P_55F_67L | 3.141057217 |
| 2175 | 541 | TTCGACTGTGAGATTTGTGGGGCATGTTTTactcagaaaggtaacctcctccgccacattaaaCTG | 46F_47D_49E_50I_52G_53A_54C_55F_67L | 3.138428936 |
| 2176 | 7944 | CTCTGGTGCGAAATATGTGGTTTTTTTTGTactcagaaaggtaacctcctccgccacattaaaCTG | 46L_47W_49E_50I_52G_53F_54F_55C_67L | 3.135222108 |
| 2177 | 1034 | TTCTTTTGTGAAATCTGTGGGGCAAGGTTTactcagaaaggtaacctcctccgccacattaaaCTG | 46F_47F_49E_50I_52G_53A_54R_55F_67L | 3.133380172 |
| 2178 | 545 | TTCGATTGTGAAATCTGTGGGGCAGGCTTTactcagaaaggtaacctcctccgccacattaaaCTG | 46F_47D_49E_50I_52G_53A_54G_55F_67L | 3.130954295 |
| 2179 | 7687 | CTCTTCTGCGAAATCTGCGGGTTCATATGTactcagaaaggtaacctcctccgccacattaaaCTG | 46L_47F_49E_50I_52G_53F_54I_55C_67L | 3.124153543 |
| 2180 | 4517 | TTCTACTGTCCTATTTGTGGGGCAGTGTTTactcagaaaggtaacctcctccgccacattaaaCTG | 46F_47Y_49P_50I_52G_53A_54V_55F_67L | 3.1179368 |
| 2181 | 3795 | TTCACTTGTCCAATCTGTGGGGCAAGCTTTactcagaaaggtaacctcctccgccacattaaaCTG | 46F_47T_49P_50I_52G_53A_54S_55F_67L | 3.116727265 |
| 2182 | 4286 | TTCTGGTGTCCACAATGTGGGGCACATTTTactcagaaaggtaacctcctccgccacattaaaCTG | 46F_47W_49P_50Q_52G_53A_54H_55F_67L | 3.115178068 |
| 2183 | 100 | TTCGCCTGTGAGGTCTGTGGGGCAGCCTTTactcagaaaggtaacctcctccgccacattaaaCTG | 46F_47A_49E_50V_52G_53A_54A_55F_67L | 3.114351461 |
| 2184 | 4337 | TTCTACTGTGATATATGTGGGGCAGTTTTTactcagaaaggtaacctcctccgccacattaaaCTG | 46F_47Y_49D_50I_52G_53A_54V_55F_67L | 3.110086105 |
| 2185 | 4304 | TTCTGGTGTCCTGTTTGTGGGGCATTTTTTactcagaaaggtaacctcctccgccacattaaaCTG | 46F_47W_49P_50V_52G_53A_54F_55F_67L | 3.106413893 |
| 2186 | 6546 | CTGTTTTGCCCCATCTGCGGCTTTCACTGTactcagaaaggtaacctcctccgccacattaaaCTG | 46L_47F_49P_50I_52G_53F_54H_55C_67L | 3.106399803 |
| 2187 | 2980 | TTCCAATGTGAAGTTTGTGGGGCAGCCTTTactcagaaaggtaacctcctccgccacattaaaCTG | 46F_47Q_49E_50V_52G_53A_54A_55F_67L | 3.100493468 |
| 2188 | 1633 | TTCCACTGTCCGATCTGTGGGGCACAGTTTactcagaaaggtaacctcctccgccacattaaaCTG | 46F_47H_49P_50I_52G_53A_54Q_55F_67L | 3.099714134 |
| 2189 | 4557 | TTCTATTGTCCAGTTTGTGGGGCAGTGTTTactcagaaaggtaacctcctccgccacattaaaCTG | 46F_47Y_49P_50V_52G_53A_54V_55F_67L | 3.098960765 |
| 2190 | 6551 | CTGTTTTGTCCCATTTGCGGGTTCAATTGCactcagaaaggtaacctcctccgccacattaaaCTG | 46L_47F_49P_50I_52G_53F_54N_55C_67L | 3.097000753 |
| 2191 | 4446 | TTCTACTGTAACATATGTGGGGCACATTTTactcagaaaggtaacctcctccgccacattaaaCTG | 46F_47Y_49N_50I_52G_53A_54H_55F_67L | 3.093756795 |
| 2192 | 4257 | TTCTGGTGTAATGTGTGTGGGGCAGTCTTTactcagaaaggtaacctcctccgccacattaaaCTG | 46F_47W_49N_50V_52G_53A_54V_55F_67L | 3.089854146 |
| 2193 | 4072 | TTCGTTTGTCCGGTCTGTGGGGCACCATTTactcagaaaggtaacctcctccgccacattaaaCTG | 46F_47V_49P_50V_52G_53A_54P_55F_67L | 3.079926509 |
| 2194 | 977 | TTCTTTTGTGACATCTGTGGGGCAGTGTTTactcagaaaggtaacctcctccgccacattaaaCTG | 46F_47F_49D_50I_52G_53A_54V_55F_67L | 3.077747176 |
| 2195 | 4279 | TTCTGGTGTCCTATTTGTGGGGCATATTTTactcagaaaggtaacctcctccgccacattaaaCTG | 46F_47W_49P_50I_52G_53A_54Y_55F_67L | 3.070657181 |
| 2196 | 1070 | TTCTTTTGTGAAGTGTGTGGGGCAATGTTTactcagaaaggtaacctcctccgccacattaaaCTG | 46F_47F_49E_50V_52G_53A_54M_55F_67L | 3.066372261 |
| 2197 | 4382 | TTCTATTGTGAGATCTGTGGGGCAGATTTTactcagaaaggtaacctcctccgccacattaaaCTG | 46F_47Y_49E_50I_52G_53A_54D_55F_67L | 3.058432615 |
| 2198 | 7694 | CTTTTCTGTGAAATTTGCGGTTTCAGGTGTactcagaaaggtaacctcctccgccacattaaaCTG | 46L_47F_49E_50I_52G_53F_54R_55C_67L | 3.058412473 |
| 2199 | 1501 | TTCCATTGTGAGATTTGTGGGGCATGCTTTactcagaaaggtaacctcctccgccacattaaaCTG | 46F_47H_49E_50I_52G_53A_54C_55F_67L | 3.05733117 |
| 2200 | 974 | TTCTTTTGTGACATTTGTGGGGCAAGATTTactcagaaaggtaacctoctccgccacattaaaCTG | 46F_47F_49D_50I_52G_53A_54R_55F_67L | 3.056912648 |
| 2201 | 2472 | TTCATGTGTGAGATTTGTGGGGCACCATTTactcagaaaggtaacctcctccgccacattaaaCTG | 46F_47M_49E_50I_52G_53A_54P_55F_67L | 3.053890881 |
| 2202 | 3972 | TTCGTCTGTAACATTTGTGGGGCACCCTTTactcagaaaggtaacctcctccgccacattaaaCTG | 46F_47V_49N_50I_52G_53A_54P_55F_67L | 3.051793471 |
| 2203 | 7688 | TTATTTTGCGAGATTTGTGGGTTTAAATGCactcagaaaggtaacctcctccgccacattaaaCTG | 46L_47F_49E_50I_52G_53G_54K_55C_67L | 3.051610685 |
| 2204 | 1065 | TTCTTTTGTGAGGTGTGTGGGGCAGGATTTactcagaaaggtaacctcctccgccacattaaaCTG | 46F_47F_49E_50V_52G_53A_54G_55F_67L | 3.05063215 |
| 2205 | 1081 | TTCTTCTGTAATATTTGTGGGGCATGCTTTactcagaaaggtaacctcctccgccacattaaaCTG | 46F_47F_49N_50I_52G_53A_54C_55F_67L | 3.049781626 |
| 2206 | 6805 | CTGTGGTGTCCAATCTGCGGTTTTGGCTGCactcagaaaggtaacctcctccgccacattaaaCTG | 46L_47W_49P_50I_52G_53F_54G_55C_67L | 3.049733709 |
| 2207 | 7735 | CTGCACTGTGAAATATGCGGCTTTTCATGCactcagaaaggtaacctcctccgccacattaaaCTG | 46L_47H_49E_50I_52G_53F_54S_55C_67L | 3.04595384 |
| 2208 | 720 | TTCGAGTGTGATATCTGTGGGGCAGCCTTTactcagaaaggtaacctcctccgccacattaaaCTG | 46F_47E_49D_50I_52G_53A_54A_55F_67L | 3.041526629 |
| 2209 | 3612 | TTCACTTGTGATATCTGTGGGGCACCCTTTactcagaaaggtaacctcctccgccacattaaaCTG | 46F_47T_49D_50I_52G_53A_54P_55F_67L | 3.041011338 |
| 2210 | 3700 | TTCACATGTGAAGTGTGTGGGGCAGCTTTTactcagaaaggtaacctcctccgccacattaaaCTG | 46F_47T_49E_50V_52G_53A_54A_55F_67L | 3.038620603 |
| 2211 | 1069 | TTCTTTTGTGAGGTGTGTGGGGCACTGTTTactcagaaaggtaacctcctccgccacattaaaCTG | 46F_47F_49E_50V_52G_53A_54L_55F_67L | 3.037718663 |
| 2212 | 552 | TTCGATTGTGAAATCTGTGGGGCACCTTTTactcagaaaggtaacctcctccgccacattaaaCTG | 46F_47D_49E_50I_52G_53A_54P_55F_67L | 3.036466484 |
| 2213 | 1197 | TTCTTTTGTCCCGTCTGTGGGGCAGTCTTTactcagaaaggtaacctcctccgccacattaaaCTG | 46F_47F_49P_50V_52G_53A_54V_55F_67L | 3.035715233 |
| 2214 | 7686 | CTGTTTTGCGAAATCTGCGGATTTCATTGTactcagaaaggtaacctcctccgccacattaaaCTG | 46L_47F_49E_50I_52G_53F_54H_55C_67L | 3.033490018 |
| 2215 | 4500 | TTCTACTGTCCAATCTGTGGGGCAGCATTTactcagaaaggtaacctcctccgccacattaaaCTG | 46F_47Y_49P_50I_52G_53A_54A_55F_67L | 3.025275403 |
| 2216 | 4166 | TTCTGGTGTGAACAGTGTGGGGCACATTTTactcagaaaggtaacctcctccgccacattaaaCTG | 46F_47W_49E_50Q_52G_53A_54H_55F_67L | 3.024101207 |
| 2217 | 1553 | TTCCATTGTGAGGTATGTGGGGCACAGTTTactcagaaaggtaacctcctccgccacattaaaCTG | 46F_47H_49E_50V_52G_53A_54Q_55F_67L | 3.023786072 |
| 2218 | 840 | TTCGAGTGTAATATATGTGGGGCAGCCTTTactcagaaaggtaacctcctccgccacattaaaCTG | 46F_47E_49N_50I_52G_53A_54A_55F_67L | 3.022800697 |
| 2219 | 4480 | TTCTATTGTAATGTTTGTGGGGCAGCCTTTactcagaaaggtaacctcctccgccacattaaaCTG | 46F_47Y_49N_50V_52G_53A_54A_55F_67L | 3.014476699 |
| 2220 | 7568 | TTCTGGTGCGAGATCTGTGGCCGCAAGTTTactcagaaaggtaacctcctccgccacattaaaCTG | 46F_47W_49E_50I_52G_53R_54K_55F_67L | 3.006459231 |
| 2221 | 546 | TTCGACTGTGAAATCTGTGGGGCACATTTTactcagaaaggtaacctcctccgccacattaaaCTG | 46F_47D_49E_50I_52G_53A_54H_55F_67L | 3.006044587 |
| 2222 | 7683 | CTGTTTTGCGAGATCTGCGGGTTCGAGTGTactcagaaaggtaacctcctccgccacattaaaCTG | 46L_47F_49E_50I_52G_53F_54E_55C_67L | 3.000709138 |
| 2223 | 4177 | TTCTGGTGTGAACAATGTGGGGCAGTCTTTactcagaaaggtaacctcctccgccacattaaaCTG | 46F_47W_49E_50Q_52G_53A_54V_55F_67L | 3.000215973 |
| 2224 | 3436 | TTCTCCTGTGAAATTTGTGGGGCAACTTTTactcagaaaggtaacctcctccgccacattaaaCTG | 46F_47S_49E_50I_52G_53A_54T_55F_67L | 2.999428695 |
| 2225 | 3940 | TTCGTCTGTGAAGTGTGTGGGGCAGCATTTactcagaaaggtaacctcctccgccacattaaaCTG | 46F_47V_49E_50V_52G_53A_54A_55F_67L | 2.997862402 |
| 2226 | 4305 | TTCTGGTGTCCCGTGTGTGGGGCAGGTTTTactcagaaaggtaacctcctccgccacattaaaCTG | 46F_47W_49P_50V_52G_53A_54G_55F_67L | 2.997240988 |
| 2227 | 4302 | TTCTGGTGTCCCGTCTGTGGGGCAGACTTTactcagaaaggtaacctcctccgccacattaaaCTG | 46F_47W_49P_50V_52G_53A_54D_55F_67L | 2.997129605 |
| 2228 | 1620 | TTCCATTGTCCAATATGTGGGGCAGCCTTTactcagaaaggtaacctcctccgccacattaaaCTG | 46F_47H_49P_50I_52G_53A_54A_55F_67L | 2.996506637 |
| 2229 | 594 | TTCGACTGTGAGGTATGTGGGGCAAGGTTTactcagaaaggtaacctcctccgccacattaaaCTG | 46F_47D_49E_50V_52G_53A_54R_55F_67L | 2.996289771 |
| 2230 | 785 | TTCGAGTGTGAGATTTGTGGGGCAGGCTTTactcagaaaggtaacctcctccgccacattaaaCTG | 46F_47E_49E_50I_52G_53A_54G_55F_67L | 2.995870298 |
| 2231 | 6831 | CTTTATTGCCCAATCTGTGGGTTTAATTGCactcagaaaggtaacctcctccgccacattaaaCTG | 46L_47Y_49P_50I_52G_53F_54N_55C_67L | 2.991473798 |
| 2232 | 4307 | TTCTGGTGTCCCGTTTGTGGGGCAATTTTTactcagaaaggtaacctcctccgccacattaaaCTG | 46F_47W_49P_50V_52G_53A_54I_55F_67L | 2.989933962 |
| 2233 | 315 | TTCTGCTGTGAGATCTGTGGGGCATCATTTactcagaaaggtaacctcctccgccacattaaaCTG | 46F_47C_49E_50I_52G_53A_54S_55F_67L | 2.988607638 |
| 2234 | 6547 | CTGTTCTGTCCAATATGTGGCTTCATCTGTactcagaaaggtaacctcctccgccacattaaaCTG | 46L_47F_49P_50I_52G_53F_54I_55C_67L | 2.985069724 |
| 2235 | 1001 | TTCTTTTGTGACGTATGTGGGGCATGCTTTactcagaaaggtaacctcctccgccacattaaaCTG | 46F_47F_49D_50V_52G_53A_54C_55F_67L | 2.983548181 |
| 2236 | 968 | TTCTTTTGTGATATCTGTGGGGCAAAGTTTactcagaaaggtaacctcctccgccacattaaaCTG | 46F_47F_49D_50I_52G_53A_54K_55F_67L | 2.974881042 |
| 2237 | 4296 | TTCTGGTGTCCGCAGTGTGGGGCAACGTTTactcagaaaggtaacctcctccgccacattaaaCTG | 46F_47W_49P_50Q_52G_53A_54T_55F_67L | 2.970903286 |
| 2238 | 4106 | TTCTGGTGTGATCAGTGTGGGGCACATTTTactcagaaaggtaacctcctccgccacattaaaCTG | 46F_47W_49D_50Q_52G_53A_54H_55F_67L | 2.96508335 |
| 2239 | 1093 | TTCTTCTGTAACATTTGTGGGGCACAATTTactcagaaaggtaacctcctccgccacattaaaCTG | 46F_47F_49N_50I_52G_53A_54Q_55F_67L | 2.963517451 |
| 2240 | 6825 | TTGTACTGTCCCATCTGTGGTTTCGGTTGCactcagaaaggtaacctcctccgccacattaaaCTG | 46L_47Y_49P_50I_52G_53F_54G_55C_67L | 2.963372479 |
| 2241 | 6554 | CTGTTTTGCCCTATTTGTGGATTCCGCTGCactcagaaaggtaacctcctccgccacattaaaCTG | 46L_47F_49P_50I_52G_53F_54R_55C_67L | 2.960533652 |
| 2242 | 4226 | TTCTGGTGTAACCAGTGTGGGGCACACTTTactcagaaaggtaacctcctccgccacattaaaCTG | 46F_47W_49N_50Q_52G_53A_54H_55F_67L | 2.958588302 |
| 2243 | 1080 | TTCTTCTGTAATATCTGTGGGGCAGCATTTactcagaaaggtaacctcctccgccacattaaaCTG | 46F_47F_49N_50I_52G_53A_54A_55F_67L | 2.957844259 |
| 2244 | 1194 | TTCTTCTGTCCTGTGTGTGGGGCACGATTTactcagaaaggtaacctcctccgccacattaaaCTG | 46F_47F_49P_50V_52G_53A_54R_55F_67L | 2.954258853 |
| 2245 | 3675 | TTCACATGTGAAATATGTGGGGCAAGTTTTactcagaaaggtaacctcctccgccacattaaaCTG | 46F_47T_49E_50I_52G_53A_54S_55F_67L | 2.953837464 |
| 2246 | 4209 | TTCTGGTGTAACATCTGTGGGGCACTGTTTactcagaaaggtaacctcctccgccacattaaaCTG | 46F_47W_49N_50I_52G_53A_54L_55F_67L | 2.952662724 |
| 2247 | 1672 | TTCCATTGTCCAGTGTGTGGGGCACCCTTTactcagaaaggtaacctcctccgccacattaaaCTG | 46F_47H_49P_50V_52G_53A_54P_55F_67L | 2.95148067 |
| 2248 | 2632 | TTCATGTGTCCAGTTTGTGGGGCACCCTTTactcagaaaggtaacctcctccgccacattaaaCTG | 46F_47M_49P_50V_52G_53A_54P_55F_67L | 2.94789005 |
| 2249 | 3792 | TTCACATGTCCCATCTGTGGGGCACCGTTTactcagaaaggtaacctcctccgccacattaaaCTG | 46F_47T_49P_50I_52G_53A_54P_55F_67L | 2.943123298 |
| 2250 | 3715 | TTCACATGTGAAGTGTGTGGGGCATCGTTTactcagaaaggtaacctcctccgccacattaaaCTG | 46F_47T_49E_50V_52G_53A_54S_55F_67L | 2.941523636 |
| 2251 | 4443 | TTCTACTGTAACATCTGTGGGGCAGAGTTTactcagaaaggtaacctcctccgccacattaaaCTG | 46F_47Y_49N_50I_52G_53A_54E_55F_67L | 2.940171113 |
| 2252 | 4125 | TTCTGGTGTGATGTATGTGGGGCAGGCTTTactcagaaaggtaacctcctccgccacattaaaCTG | 46F_47W_49D_50V_52G_53A_54G_55F_67L | 2.9362124 |
| 2253 | 4334 | TTCTATTGTGATATCTGTGGGGCAAGATTTactcagaaaggtaacctcctccgccacattaaaCTG | 46F_47Y_49D_50I_52G_53A_54R_55F_67L | 2.934773301 |
| 2254 | 3067 | TTCCAGTGTCCTATCTGTGGGGCAATTTTTactcagaaaggtaacctcctccgccacattaaaCTG | 46F_47Q_49P_50I_52G_53A_54I_55F_67L | 2.933461393 |
| 2255 | 4372 | TTCTACTGTGATGTCTGTGGGGCACCGTTTactcagaaaggtaacctcctccgccacattaaaCTG | 46F_47Y_49D_50V_52G_53A_54P_55F_67L | 2.929447987 |
| 2256 | 4264 | TTCTGGTGTCCAATCTGTGGGGCATTCTTTactcagaaaggtaacctcctccgccacattaaaCTG | 46F_47W_49P_50I_52G_53A_54F_55F_67L | 2.928853915 |
| 2257 | 7896 | CTGTCCTGCGAAATCTGCGGATTCACATGCactcagaaaggtaacctcctccgccacattaaaCTG | 46L_47S_49E_50I_52G_53F_54T_55C_67L | 2.927159738 |
| 2258 | 551 | TTCGATTGTGAGATCTGTGGGGCAAATTTTactcagaaaggtaacctcctccgccacattaaaCTG | 46F_47D_49E_50I_52G_53A_54N_55F_67L | 2.92164276 |
| 2259 | 2892 | TTCCAGTGTGATATTTGTGGGGCACCCTTTactcagaaaggtaacctcctccgccacattaaaCTG | 46F_47Q_49D_50I_52G_53A_54P_55F_67L | 2.919238068 |
| 2260 | 4252 | TTCTGGTGTAATGTTTGTGGGGCACCTTTTactcagaaaggtaacctcctccgccacattaaaCTG | 46F_47W_49N_50V_52G_53A_54P_55F_67L | 2.91811761 |
| 2261 | 4124 | TTCTGGTGTGATGTATGTGGGGCATTTTTTactcagaaaggtaacctcctccgccacattaaaCTG | 46F_47W_49D_50V_52G_53A_54F_55F_67L | 2.913346889 |
| 2262 | 4481 | TTCTATTGTAACGTGTGTGGGGCATGCTTTactcagaaaggtaacctcctccgccacattaaaCTG | 46F_47Y_49N_50V_52G_53A_54C_55F_67L | 2.905598562 |
| 2263 | 4116 | TTCTGGTGTGATCAGTGTGGGGCAACCTTTactcagaaaggtaacctcctccgccacattaaaCTG | 46F_47W_49D_50Q_52G_53A_54T_55F_67L | 2.90411395 |
| 2264 | 4507 | TTCTATTGTCCTATATGTGGGGCAATTTTTactcagaaaggtaacctcctccgccacattaaaCTG | 46F_47Y_49P_50I_52G_53A_54I_55F_67L | 2.903224152 |
| 2265 | 1091 | TTCTTTTGTAATATTTGTGGGGCAAATTTTactcagaaaggtaacctcctccgccacattaaaCTG | 46F_47F_49N_50I_52G_53A_54N_55F_67L | 2.902583495 |
| 2266 | 4495 | TTCTACTGTAACGTTTGTGGGGCAAGCTTTactcagaaaggtaacctcctccgccacattaaaCTG | 46F_47Y_49N_50V_52G_53A_54S_55F_67L | 2.901675749 |
| 2267 | 1541 | TTCCATTGTGAGGTGTGTGGGGCATGCTTTactcagaaaggtaacctcctccgccacattaaaCTG | 46F_47H_49E_50V_52G_53A_54C_55F_67L | 2.901449683 |
| 2268 | 1552 | TTCCACTGTGAGGTGTGTGGGGCACCTTTTactcagaaaggtaacctcctccgccacattaaaCTG | 46F_47H_49E_50V_52G_53A_54P_55F_67L | 2.901020553 |
| 2269 | 4400 | TTCTATTGTGAGCAGTGTGGGGCAGCCTTTactcagaaaggtaacctcctccgccacattaaaCTG | 46F_47Y_49E_50Q_52G_53A_54A_55F_67L | 2.898540415 |
| 2270 | 4368 | TTCTATTGTGACGTATGTGGGGCAAAATTTactcagaaaggtaacctcctccgccacattaaaCTG | 46F_47Y_49D_50V_52G_53A_54K_55F_67L | 2.897494687 |
| 2271 | 3012 | TTCCAATGTAATATATGTGGGGCACCCTTTactcagaaaggtaacctcctccgccacattaaaCTG | 46F_47Q_49N_50I_52G_53A_54P_55F_67L | 2.893156671 |
| 2272 | 4415 | TTCTACTGTGAGCAATGTGGGGCAAGTTTTactcagaaaggtaacctcctccgccacattaaaCTG | 46F_47Y_49E_50Q_52G_53A_54S_55F_67L | 2.887648206 |
| 2273 | 480 | TTCGATTGTGATATCTGTGGGGCAGCTTTTactcagaaaggtaacctcctccgccacattaaaCTG | 46F_47D_49D_50I_52G_53A_54A_55F_67L | 2.886638993 |
| 2274 | 4509 | TTCTATTGTCCAATCTGTGGGGCATTGTTTactcagaaaggtaacctcctccgccacattaaaCTG | 46F_47Y_49P_50I_52G_53A_54L_55F_67L | 2.885635103 |
| 2275 | 1095 | TTCTTTTGTAATATTTGTGGGGCATCCTTTactcagaaaggtaacctcctccgccacattaaaCTG | 46F_47F_49N_50I_52G_53A_54S_55F_67L | 2.882023704 |
| 2276 | 4259 | TTCTGGTGTAATGTCTGTGGGGCATATTTTactcagaaaggtaacctcctccgccacattaaaCTG | 46F_47W_49N_50V_52G_53A_54Y_55F_67L | 2.87887273 |
| 2277 | 1812 | TTCATCTGTAATATTTGTGGGGCACCCTTTactcagaaaggtaacctcctccgccacattaaaCTG | 46F_47I_49N_50I_52G_53A_54P_55F_67L | 2.871636317 |
| 2278 | 6755 | CTTTCGTGTCCCATTTGCGGTTTCTCATGCactcagaaaggtaacctcctccgccacattaaaCTG | 46L_47S_49P_50I_52G_53F_54S_55C_67L | 2.860257446 |
| 2279 | 4384 | TTCTATTGTGAGATCTGTGGGGCATTTTTTactcagaaaggtaacctcctccgccacattaaaCTG | 46F_47Y_49E_50I_52G_53A_54F_55F_67L | 2.859488312 |
| 2280 | 3732 | TTCACATGTAATATCTGTGGGGCACCCTTTactcagaaaggtaacctcctccgccacattaaaCTG | 46F_47T_49N_50I_52G_53A_54P_55F_67L | 2.855158088 |
| 2281 | 1673 | TTCCATTGTCCAGTGTGTGGGGCACAATTTactcagaaaggtaacctcctccgccacattaaaCTG | 46F_47H_49P_50V_52G_53A_54Q_55F_67L | 2.854516778 |
| 2282 | 825 | TTCGAGTGTGAGGTATGTGGGGCAGGCTTTactcagaaaggtaacctcctccgccacattaaaCTG | 46F_47E_49E_50V_52G_53A_54G_55F_67L | 2.852686056 |
| 2283 | 966 | TTCTTTTGTGACATTTGTGGGGCACATTTTactcagaaaggtaacctcctccgccacattaaaCTG | 46F_47F_49D_50I_52G_53A_54H_55F_67L | 2.849836992 |
| 2284 | 600 | TTCGACTGTAACATCTGTGGGGCAGCATTTactcagaaaggtaacctcctccgccacattaaaCTG | 46F_47D_49N_50I_52G_53A_54A_55F_67L | 2.845317096 |
| 2285 | 6824 | CTTTACTGTCCCATATGCGGGTTTTTCTGTactcagaaaggtaacctcctccgccacattaaaCTG | 46L_47Y_49P_50I_52G_53F_54F_55C_67L | 2.841440213 |
| 2286 | 3435 | TTCTCGTGTGAGATCTGTGGGGCATCGTTTactcagaaaggtaacctcctccgccacattaaaCTG | 46F_47S_49E_50I_52G_53A_54S_55F_67L | 2.840748645 |
| 2287 | 3421 | TTCAGCTGTGAAATCTGTGGGGCATGCTTTactcagaaaggtaacctcctccgccacattaaaCTG | 46F_47S_49E_50I_52G_53A_54C_55F_67L | 2.828393098 |
| 2288 | 4182 | TTCTGGTGTGAAGTTTGTGGGGCAGACTTTactcagaaaggtaacctcctccgccacattaaaCTG | 46F_47W_49E_50V_52G_53A_54D_55F_67L | 2.825103184 |
| 2289 | 3360 | TTCAGCTGTGATATTTGTGGGGCAGCCTTTactcagaaaggtaacctcctccgccacattaaaCTG | 46F_47S_49D_50I_52G_53A_54A_55F_67L | 2.822859337 |
| 2290 | 556 | TTCGACTGTGAGATCTGTGGGGCAACCTTTactcagaaaggtaacctcctccgccacattaaaCTG | 46F_47D_49E_50I_52G_53A_54T_55F_67L | 2.821375189 |
| 2291 | 3172 | TTCAGATGTGACGTTTGTGGGGCACCGTTTactcagaaaggtaacctcctccgccacattaaaCTG | 46F_47R_49D_50V_52G_53A_54P_55F_67L | 2.81994179 |
| 2292 | 4543 | TTCTATTGTCCCGTGTGTGGGGCAGAGTTTactcagaaaggtaacctcctccgccacattaaaCTG | 46F_47Y_49P_50V_52G_53A_54E_55F_67L | 2.8189892 |
| 2293 | 1159 | TTCTTTTGTCCGATTTGTGGGGCATACTTTactcagaaaggtaacctcctccgccacattaaaCTG | 46F_47F_49P_50I_52G_53A_54Y_55F_67L | 2.817367609 |
| 2294 | 2832 | TTCAACTGTCCTATTTGTGGGGCACCTTTTactcagaaaggtaacctcctccgccacattaaaCTG | 46F_47N_49P_50I_52G_53A_54P_55F_67L | 2.815229545 |
| 2295 | 3955 | TTCGTATGTGAGGTTTGTGGGGCATCCTTTactcagaaaggtaacctcctccgccacattaaaCTG | 46F_47V_49E_50V_52G_53A_54S_55F_67L | 2.814703177 |
| 2296 | 4294 | TTCTGGTGTCCTCAATGTGGGGCACGTTTTactcagaaaggtaacctcctccgccacattaaaCTG | 46F_47W_49P_50Q_52G_53A_54R_55F_67L | 2.812020523 |
| 2297 | 7971 | CTCTACTGTGAAATTTGTGGATTCAATTGTactcagaaaggtaacctcctccgccacattaaaCTG | 46L_47Y_49E_50I_52G_53F_54N_55C_67L | 2.807166499 |
| 2298 | 3905 | TTCGTGTGTGAAATTTGTGGGGCAGGTTTTactcagaaaggtaacctcctccgccacattaaaCTG | 46F_47V_49E_50I_52G_53A_54G_55F_67L | 2.804875858 |
| 2299 | 4269 | TTCTGGTGTCCCATTTGTGGGGCACTGTTTactcagaaaggtaacctcctccgccacattaaaCTG | 46F_47W_49P_50I_52G_53A_54L_55F_67L | 2.804733527 |
| 2300 | 6795 | CTGGTGTGCCCCATCTGCGGCTTCTCTTGTactcagaaaggtaacctcctccgccacattaaaCTG | 46L_47V_49P_50I_52G_53F_54S_55C_67L | 2.801479011 |
| 2301 | 6550 | CTTTTCTGCCCAATCTGCGGATTCATGTGTactcagaaaggtaacctcctccgccacattaaaCTG | 46L_47F_49P_50I_52G_53F_54M_55C_67L | 2.799620696 |
| 2302 | 4377 | TTCTATTGTGATGTATGTGGGGCAGTCTTTactcagaaaggtaacctcctccgccacattaaaCTG | 46F_47Y_49D_50V_52G_53A_54V_55F_67L | 2.799569192 |
| 2303 | 1192 | TTCTTTTGTCCAGTGTGTGGGGCACCTTTTactcagaaaggtaacctcctccgccacattaaaCTG | 46F_47F_49P_50V_52G_53A_54P_55F_67L | 2.797177413 |
| 2304 | 3820 | TTCACCTGTCCCGTGTGTGGGGCAGCATTTactcagaaaggtaacctcctccgccacattaaaCTG | 46F_47T_49P_50V_52G_53A_54A_55F_67L | 2.797016795 |
| 2305 | 1146 | TTCTTCTGTCCTATCTGTGGGGCACACTTTactcagaaaggtaacctcctccgccacattaaaCTG | 46F_47F_49P_50I_52G_53A_54H_55F_67L | 2.791510754 |
| 2306 | 4508 | TTCTATTGTCCCATCTGTGGGGCAAAGTTTactcagaaaggtaacctcctccgccacattaaaCTG | 46F_47Y_49P_50I_52G_53A_54K_55F_67L | 2.789420809 |
| 2307 | 1572 | TTCCATTGTAACATATGTGGGGCACCTTTTactcagaaaggtaacctcctccgccacattaaaCTG | 46F_47H_49N_50I_52G_53A_54P_55F_67L | 2.781420635 |
| 2308 | 7616 | TTAGCCTGCGAGATCTGCGGGTTTACCTGTactcagaaaggtaacctcctccgccacattaaaCTG | 46L_47A_49E_50I_52G_53F_54T_55C_67L | 2.778800314 |
| 2309 | 4168 | TTCTGGTGTGAACAGTGTGGGGCAAAGTTTactcagaaaggtaacctcctccgccacattaaaCTG | 46F_47W_49E_50Q_52G_53A_54K_55F_67L | 2.774208308 |
| 2310 | 7574 | TTCTGGTGCGAAATCTGTGGGCGGAGATTTactcagaaaggtaacctcctccgccacattaaaCTG | 46F_47W_49E_50I_52G_53R_54R_55F_67L | 2.772314311 |
| 2311 | 4158 | TTCTGGTGTGAGATATGTGGGGCATGGTTTactcagaaaggtaacctcctccgccacattaaaCTG | 46F_47W_49E_50I_52G_53A_54W_55F_67L | 2.771150962 |
| 2312 | 4262 | TTCTGGTGTCCGATCTGTGGGGCAGATTTTactcagaaaggtaacctcctccgccacattaaaCTG | 46F_47W_49P_50I_52G_53A_54D_55F_67L | 2.76938821 |
| 2313 | 4493 | TTCTACTGTAATGTATGTGGGGCACAGTTTactcagaaaggtaacctcctccgccacattaaaCTG | 46F_47Y_49N_50V_52G_53A_54Q_55F_67L | 2.768513166 |
| 2314 | 2940 | TTCCAATGTGAAATCTGTGGGGCAGCATTTactcagaaaggtaacctcctccgccacattaaaCTG | 46F_47Q_49E_50I_52G_53A_54A_55F_67L | 2.768151963 |
| 2315 | 1635 | TTCCACTGTCCTATCTGTGGGGCATCCTTTactcagaaaggtaacctcctccgccacattaaaCTG | 46F_47H_49P_50I_52G_53A_54S_55F_67L | 2.765773459 |
| 2316 | 3892 | TTCGTGTGTGATGTATGTGGGGCACCCTTTactcagaaaggtaacctcctccgccacattaaaCTG | 46F_47V_49D_50V_52G_53A_54P_55F_67L | 2.764273095 |
| 2317 | 4413 | TTCTACTGTGAACAGTGTGGGGCACAATTTactcagaaaggtaacctcctccgccacattaaaCTG | 46F_47Y_49E_50Q_52G_53A_54Q_55F_67L | 2.76319992 |
| 2318 | 3832 | TTCACTTGTCCCGTCTGTGGGGCACCGTTTactcagaaaggtaacctcctccgccacattaaaCTG | 46F_47T_49P_50V_52G_53A_54P_55F_67L | 2.762497963 |
| 2319 | 1014 | TTCTTTTGTGACGTGTGTGGGGCACGTTTTactcagaaaggtaacctcctccgccacattaaaCTG | 46F_47F_49D_50V_52G_53A_54R_55F_67L | 2.759296234 |
| 2320 | 4132 | TTCTGGTGTGACGTGTGTGGGGCACCGTTTactcagaaaggtaacctcctccgccacattaaaCTG | 46F_47W_49D_50V_52G_53A_54P_55F_67L | 2.757950552 |
| 2321 | 1186 | TTCTTTTGTCCTGTCTGTGGGGCACATTTTactcagaaaggtaacctcctccgccacattaaaCTG | 46F_47F_49P_50V_52G_53A_54H_55F_67L | 2.754403967 |
| 2322 | 3600 | TTCACATGTGATATCTGTGGGGCAGCCTTTactcagaaaggtaacctcctccgccacattaaaCTG | 46F_47T_49D_50I_52G_53A_54A_55F_67L | 2.751185556 |
| 2323 | 3433 | TTCTCGTGTGAAATCTGTGGGGCACAGTTTactcagaaaggtaacctcctccgccacattaaaCTG | 46F_47S_49E_50I_52G_53A_54Q_55F_67L | 2.749718327 |
| 2324 | 1575 | TTCCATTGTAACATTTGTGGGGCATCATTTactcagaaaggtaacctcctccgccacattaaaCTG | 46F_47H_49N_50I_52G_53A_54S_55F_67L | 2.749643286 |
| 2325 | 7736 | TTGCATTGCGAAATATGTGGCTTCACTTGTactcagaaaggtaacctcctccgccacattaaaCTG | 46L_47H_49E_50I_52G_53F_54T_55C_67L | 2.749383843 |
| 2326 | 852 | TTCGAGTGTAATATTTGTGGGGCACCCTTTactcagaaaggtaacctcctccgccacattaaaCTG | 46F_47E_49N_50I_52G_53A_54P_55F_67L | 2.749234817 |
| 2327 | 495 | TTCGATTGTGATATTTGTGGGGCAAGTTTTactcagaaaggtaacctcctccgccacattaaaCTG | 46F_47D_49D_50I_52G_53A_54S_55F_67L | 2.747963391 |
| 2328 | 1660 | TTCCACTGTCCTGTTTGTGGGGCAGCATTTactcagaaaggtaacctcctccgccacattaaaCTG | 46F_47H_49P_50V_52G_53A_54A_55F_67L | 2.745743542 |
| 2329 | 1079 | TTCTTCTGTGAGGTGTGTGGGGCATACTTTactcagaaaggtaacctcctccgccacattaaaCTG | 46F_47F_49E_50V_52G_53A_54Y_55F_67L | 2.74308509 |
| 2330 | 4325 | TTCTATTGTGATATCTGTGGGGCAGGGTTTactcagaaaggtaacctcctccgccacattaaaCTG | 46F_47Y_49D_50I_52G_53A_54G_55F_67L | 2.740562746 |
| 2331 | 4129 | TTCTGGTGTGACGTCTGTGGGGCACTGTTTactcagaaaggtaacctcctccgccacattaaaCTG | 46F_47W_49D_50V_52G_53A_54L_55F_67L | 2.732883719 |
| 2332 | 6775 | CTCACCTGCCCAATCTGCGGGTTCAGCTGTactcagaaaggtaacctcctccgccacattaaaCTG | 46L_47T_49P_50I_52G_53F_54S_55C_67L | 2.729829086 |
| 2333 | 1154 | TTCTTTTGTCCAATCTGTGGGGCACGCTTTactcagaaaggtaacctcctccgccacattaaaCTG | 46F_47F_49P_50I_52G_53A_54R_55F_67L | 2.72394228 |
| 2334 | 355 | TTCTGTTGTGAGGTCTGTGGGGCAAGTTTTactcagaaaggtaacctcctccgccacattaaaCTG | 46F_47C_49E_50V_52G_53A_54S_55F_67L | 2.717905688 |
| 2335 | 4374 | TTCTACTGTGACGTGTGTGGGGCACGTTTTactcagaaaggtaacctcctccgccacattaaaCTG | 46F_47Y_49D_50V_52G_53A_54R_55F_67L | 2.714854922 |
| 2336 | 557 | TTCGATTGTGAGATATGTGGGGCAGTATTTactcagaaaggtaacctcctccgccacattaaaCTG | 46F_47D_49E_50I_52G_53A_54V_55F_67L | 2.714381423 |
| 2337 | 1440 | TTCCACTGTGATATTTGTGGGGCAGCATTTactcagaaaggtaacctcctccgccacattaaaCTG | 46F_47H_49D_50I_52G_53A_54A_55F_67L | 2.711925361 |
| 2338 | 3100 | TTCCAGTGTCCAGTTTGTGGGGCAGCCTTTactcagaaaggtaacctcctccgccacattaaaCTG | 46F_47Q_49P_50V_52G_53A_54A_55F_67L | 2.706639781 |
| 2339 | 3476 | TTCAGCTGTGAGGTTTGTGGGGCAACTTTTactcagaaaggtaacctcctccgccacattaaaCTG | 46F_47S_49E_50V_52G_53A_54T_55F_67L | 2.705087223 |
| 2340 | 1096 | TTCTTCTGTAATATTTGTGGGGCAACCTTTactcagaaaggtaacctcctccgccacattaaaCTG | 46F_47F_49N_50I_52G_53A_54T_55F_67L | 2.70357032 |
| 2341 | 2987 | TTCCAGTGTGAGGTCTGTGGGGCAATATTTactcagaaaggtaacctcctccgccacattaaaCTG | 46F_47Q_49E_50V_52G_53A_54I_55F_67L | 2.703151441 |
| 2342 | 2412 | TTCATGTGTGACATTTGTGGGGCACCCTTTactcagaaaggtaacctcctccgccacattaaaCTG | 46F_47M_49D_50I_52G_53A_54P_55F_67L | 2.702234154 |
| 2343 | 1147 | TTCTTTTGTCCCATCTGTGGGGCAATCTTTactcagaaaggtaacctcctccgccacattaaaCTG | 46F_47F_49P_50I_52G_53A_54I_55F_67L | 2.700518918 |
| 2344 | 6834 | CTTTATTGTCCCATCTGTGGATTTCGGTGTactcagaaaggtaacctcctccgccacattaaaCTG | 46L_47Y_49P_50I_52G_53F_54R_55C_67L | 2.684739179 |
| 2345 | 6545 | CTCTTCTGCCCAATCTGTGGCTTTGGATGTactcagaaaggtaacctcctccgccacattaaaCTG | 46L_47F_49P_50I_52G_53F_54G_55C_67L | 2.683223624 |
| 2346 | 432 | TTCTGCTGTCCGATCTGTGGGGCACCTTTTactcagaaaggtaacctcctccgccacattaaaCTG | 46F_47C_49P_50I_52G_53A_54P_55F_67L | 2.682978698 |
| 2347 | 4445 | TTCTATTGTAACATTTGTGGGGCAGGCTTTactcagaaaggtaacctcctccgccacattaaaCTG | 46F_47Y_49N_50I_52G_53A_54G_55F_67L | 2.67794712 |
| 2348 | 591 | TTCGATTGTGAAGTCTGTGGGGCAAATTTTactcagaaaggtaacctcctccgccacattaaaCTG | 46F_47D_49E_50V_52G_53A_54N_55F_67L | 2.677654733 |
| 2349 | 3252 | TTCAGGTGTAACATTTGTGGGGCACCGTTTactcagaaaggtaacctcctccgccacattaaaCTG | 46F_47R_49N_50I_52G_53A_54P_55F_67L | 2.675829465 |
| 2350 | 2957 | TTCCAGTGTGAAATTTGTGGGGCAGTTTTTactcagaaaggtaacctcctccgccacattaaaCTG | 46F_47Q_49E_50I_52G_53A_54V_55F_67L | 2.675147197 |
| 2351 | 3900 | TTCGTGTGTGAAATCTGTGGGGCAGCATTTactcagaaaggtaacctcctccgccacattaaaCTG | 46F_47V_49E_50I_52G_53A_54A_55F_67L | 2.674806249 |
| 2352 | 4550 | TTCTATTGTCCAGTGTGTGGGGCAATGTTTactcagaaaggtaacctcctccgccacattaaaCTG | 46F_47Y_49P_50V_52G_53A_54M_55F_67L | 2.667780468 |
| 2353 | 4536 | TTCTATTGTCCACAGTGTGGGGCAACCTTTactcagaaaggtaacctcctccgccacattaaaCTG | 46F_47Y_49P_50Q_52G_53A_54T_55F_67L | 2.667170015 |
| 2354 | 115 | TTCGCTTGTGAGGTGTGTGGGGCAAGTTTTactcagaaaggtaacctcctccgccacattaaaCTG | 46F_47A_49E_50V_52G_53A_54S_55F_67L | 2.66475205 |
| 2355 | 593 | TTCGATTGTGAGGTATGTGGGGCACAGTTTactcagaaaggtaacctcctccgccacattaaaCTG | 46F_47D_49E_50V_52G_53A_54Q_55F_67L | 2.663724086 |
| 2356 | 312 | TTCTGCTGTGAGATTTGTGGGGCACCTTTTactcagaaaggtaacctcctccgccacattaaaCTG | 46F_47C_49E_50I_52G_53A_54P_55F_67L | 2.661980579 |
| 2357 | 6559 | CTGTTTTGTCCAATCTGCGGGTTCTATTGCactcagaaaggtaacctcctccgccacattaaaCTG | 46L_47F_49P_50I_52G_53F_54Y_55C_67L | 2.661184944 |
| 2358 | 2955 | TTCCAATGTGAGATCTGTGGGGCAAGCTTTactcagaaaggtaacctcctccgccacattaaaCTG | 46F_47Q_49E_50I_52G_53A_54S_55F_67L | 2.659747734 |
| 2359 | 313 | TTCTGTTGTGAGATATGTGGGGCACAATTTactcagaaaggtaacctcctccgccacattaaaCTG | 46F_47C_49E_50I_52G_53A_54Q_55F_67L | 2.659437726 |
| 2360 | 7964 | CTGTACTGCGAAATCTGCGGGTTTTTTTGCactcagaaaggtaacctcctccgccacattaaaCTG | 46L_47Y_49E_50I_52G_53F_54F_55C_67L | 2.653125901 |
| 2361 | 1149 | TTCTTTTGTCCTATCTGTGGGGCACTATTTactcagaaaggtaacctcctccgccacattaaaCTG | 46F_47F_49P_50I_52G_53A_54L_55F_67L | 2.649601496 |
| 2362 | 7915 | TTGACTTGCGAGATATGTGGATTCTCCTGCactcagaaaggtaacctcctccgccacattaaaCTG | 46L_47T_49E_50I_52G_53F_54S_55C_67L | 2.648591686 |
| 2363 | 7636 | CTTTGCTGTGAGATCTGTGGTTTTACATGTactcagaaaggtaacctcctccgccacattaaaCTG | 46L_47C_49E_50I_52G_53F_54T_55C_67L | 2.644496156 |
| 2364 | 316 | TTCTGCTGTGAGATCTGTGGGGCAACCTTTactcagaaaggtaacctcctccgccacattaaaCTG | 46F_47C_49E_50I_52G_53A_54T_55F_67L | 2.640540363 |
| 2365 | 6581 | CTGCACTGCCCAATTTGCGGGTTCTGCTGTactcagaaaggtaacctcctccgccacattaaaCTG | 46L_47H_49P_50I_52G_53F_54C_55C_67L | 2.636593845 |
| 2366 | 3437 | TTCTCTTGTGAAATTTGTGGGGCAGTCTTTactcagaaaggtaacctcctccgccacattaaaCTG | 46F_47S_49E_50I_52G_53A_54V_55F_67L | 2.635204925 |
| 2367 | 4450 | TTCTATTGTAACATCTGTGGGGCAATGTTTactcagaaaggtaacctcctccgccacattaaaCTG | 46F_47Y_49N_50I_52G_53A_54M_55F_67L | 2.635082753 |
| 2368 | 1056 | TTCTTCTGTGAGCAATGTGGGGCAACGTTTactcagaaaggtaacctcctccgccacattaaaCTG | 46F_47F_49E_50Q_52G_53A_54T_55F_67L | 2.630053354 |
| 2369 | 4330 | TTCTATTGTGATATCTGTGGGGCAATGTTTactcagaaaggtaacctcctccgccacattaaaCTG | 46F_47Y_49D_50I_52G_53A_54M_55F_67L | 2.629560705 |
| 2370 | 1516 | TTCCACTGTGAGATCTGTGGGGCAACCTTTactcagaaaggtaacctcctccgccacattaaaCTG | 46F_47H_49E_50I_52G_53A_54T_55F_67L | 2.627173479 |
| 2371 | 4422 | TTCTACTGTGAAGTGTGTGGGGCAGATTTTactcagaaaggtaacctcctccgccacattaaaCTG | 46F_47Y_49E_50V_52G_53A_54D_55F_67L | 2.623282754 |
| 2372 | 615 | TTCGATTGTAATATATGTGGGGCAAGCTTTactcagaaaggtaacctcctccgccacattaaaCTG | 46F_47D_49N_50I_52G_53A_54S_55F_67L | 2.62138724 |
| 2373 | 1260 | TTCGGATGTGAGATCTGTGGGGCAGCGTTTactcagaaaggtaacctcctccgccacattaaaCTG | 46F_47G_49E_50I_52G_53A_54A_55F_67L | 2.621144988 |
| 2374 | 586 | TTCGACTGTGAGGTGTGTGGGGCACACTTTactcagaaaggtaacctcctccgccacattaaaCTG | 46F_47D_49E_50V_52G_53A_54H_55F_67L | 2.620173756 |
| 2375 | 4548 | TTCTACTGTCCCGTCTGTGGGGCAAAATTTactcagaaaggtaacctcctccgccacattaaaCTG | 46F_47Y_49P_50V_52G_53A_54K_55F_67L | 2.619477861 |
| 2376 | 3480 | TTCTCCTGTAACATCTGTGGGGCAGCATTTactcagaaaggtaacctcctccgccacattaaaCTG | 46F_47S_49N_50I_52G_53A_54A_55F_67L | 2.616899304 |
| 2377 | 4243 | TTCTGGTGTAACGTGTGTGGGGCAGAGTTTactcagaaaggtaacctcctccgccacattaaaCTG | 46F_47W_49N_50V_52G_53A_54E_55F_67L | 2.616447402 |
| 2378 | 4510 | TTCTACTGTCCCATTTGTGGGGCAATGTTTactcagaaaggtaacctcctccgccacattaaaCTG | 46F_47Y_49P_50I_52G_53A_54M_55F_67L | 2.616101532 |
| 2379 | 1380 | TTCGGGTGTCCCATATGTGGGGCAGCTTTTactcagaaaggtaacctcctccgccacattaaaCTG | 46F_47G_49P_50I_52G_53A_54A_55F_67L | 2.615434602 |
| 2380 | 75 | TTCGCCTGTGAGATCTGTGGGGCATCATTTactcagaaaggtaacctcctccgccacattaaaCTG | 46F_47A_49E_50I_52G_53A_54S_55F_67L | 2.611409241 |
| 2381 | 3432 | TTCTCCTGTGAGATTTGTGGGGCACCCTTTactcagaaaggtaacctcctccgccacattaaaCTG | 46F_47S_49E_50I_52G_53A_54P_55F_67L | 2.610200069 |
| 2382 | 6776 | CTGACGTGTCCCATTTGTGGGTTTACGTGTactcagaaaggtaacctcctccgccacattaaaCTG | 46L_47T_49P_50I_52G_53F_54T_55C_67L | 2.606310107 |
| 2383 | 1300 | TTCGGGTGTGAGGTCTGTGGGGCAGCTTTTactcagaaaggtaacctcctccgccacattaaaCTG | 46F_47G_49E_50V_52G_53A_54A_55F_67L | 2.606212562 |
| 2384 | 120 | TTCGCCTGTAACATTTGTGGGGCAGCATTTactcagaaaggtaacctcctccgccacattaaaCTG | 46F_47A_49N_50I_52G_53A_54A_55F_67L | 2.603227724 |
| 2385 | 6596 | CTGCACTGCCCTATCTGTGGCTTCACATGTactcagaaaggtaacctcctccgccacattaaaCTG | 46L_47H_49P_50I_52G_53F_54T_55C_67L | 2.602378472 |
| 2386 | 353 | TTCTGCTGTGAGGTGTGTGGGGCACAGTTTactcagaaaggtaacctcctccgccacattaaaCTG | 46F_47C_49E_50V_52G_53A_54Q_55F_67L | 2.600114368 |
| 2387 | 6809 | CTTTGGTGCCCCATCTGTGGTTTTCTTTGTactcagaaaggtaacctcctccgccacattaaaCTG | 46L_47W_49P_50I_52G_53F_54L_55C_67L | 2.594260748 |
| 2388 | 435 | TTCTGTTGTCCAATTTGTGGGGCATCCTTTactcagaaaggtaacctcctccgccacattaaaCTG | 46F_47C_49P_50I_52G_53A_54S_55F_67L | 2.593408051 |
| 2389 | 6828 | CTGTATTGCCCTATTTGCGGTTTCAAGTGTactcagaaaggtaacctcctccgccacattaaaCTG | 46L_47Y_49P_50I_52G_53F_54K_55C_67L | 2.591380924 |
| 2390 | 3461 | TTCAGCTGTGAAGTCTGTGGGGCATGCTTTactcagaaaggtaacctcctccgccacattaaaCTG | 46F_47S_49E_50V_52G_53A_54C_55F_67L | 2.590962023 |
| 2391 | 6548 | CTGTTTTGTCCCATCTGTGGCTTCAAGTGTactcagaaaggtaacctcctccgccacattaaaCTG | 46L_47F_49P_50I_52G_53F_54K_55C_67L | 2.590582027 |
| 2392 | 1083 | TTCTTCTGTAATATTTGTGGGGCAGAGTTTactcagaaaggtaacctcctccgccacattaaaCTG | 46F_47F_49N_50I_52G_53A_54E_55F_67L | 2.589239913 |
| 2393 | 4247 | TTCTGGTGTAATGTGTGTGGGGCAATCTTTactcagaaaggtaacctcctccgccacattaaaCTG | 46F_47W_49N_50V_52G_53A_54I_55F_67L | 2.584165052 |
| 2394 | 1621 | TTCCACTGTCCTATTTGTGGGGCATGCTTTactcagaaaggtaacctcctccgccacattaaaCTG | 46F_47H_49P_50I_52G_53A_54C_55F_67L | 2.583185377 |
| 2395 | 6475 | TTGGCGTGTCCAATTTGCGGATTCTCATGTactcagaaaggtaacctcctccgccacattaaaCTG | 46L_47A_49P_50I_52G_53F_54S_55C_67L | 2.581013889 |
| 2396 | 2947 | TTCCAATGTGAGATCTGTGGGGCAATCTTTactcagaaaggtaacctcctccgccacattaaaCTG | 46F_47Q_49E_50I_52G_53A_54I_55F_67L | 2.577972467 |
| 2397 | 543 | TTCGACTGTGAGATTTGTGGGGCAGAGTTTactcagaaaggtaacctcctccgccacattaaaCTG | 46F_47D_49E_50I_52G_53A_54E_55F_67L | 2.574092753 |
| 2398 | 6780 | CTCGTTTGCCCAATTTGTGGGTTTGCTTGTactcagaaaggtaacctcctccgccacattaaaCTG | 46L_47V_49P_50I_52G_53F_54A_55C_67L | 2.57363107 |
| 2399 | 4424 | TTCTATTGTGAGGTCTGTGGGGCATTCTTTactcagaaaggtaacctcctccgccacattaaaCTG | 46F_47Y_49E_50V_52G_53A_54F_55F_67L | 2.560239186 |
| 2400 | 4547 | TTCTACTGTCCAGTGTGTGGGGCAATCTTTactcagaaaggtaacctcctccgccacattaaaCTG | 46F_47Y_49P_50V_52G_53A_54I_55F_67L | 2.559353724 |
| 2401 | 832 | TTCGAATGTGAGGTGTGTGGGGCACCTTTTactcagaaaggtaacctcctccgccacattaaaCTG | 46F_47E_49E_50V_52G_53A_54P_55F_67L | 2.559327078 |
| 2402 | 4504 | TTCTACTGTCCAATCTGTGGGGCATTTTTTactcagaaaggtaacctcctccgccacattaaaCTG | 46F_47Y_49P_50I_52G_53A_54F_55F_67L | 2.557722866 |
| 2403 | 1455 | TTCCATTGTGACATTTGTGGGGCAAGCTTTactcagaaaggtaacctcctccgccacattaaaCTG | 46F_47H_49D_50I_52G_53A_54S_55F_67L | 2.556991883 |
| 2404 | 3540 | TTCAGCTGTCCAATATGTGGGGCAGCTTTTactcagaaaggtaacctcctccgccacattaaaCTG | 46F_47S_49P_50I_52G_53A_54A_55F_67L | 2.551614157 |
| 2405 | 732 | TTCGAGTGTGATATCTGTGGGGCACCGTTTactcagaaaggtaacctcctccgccacattaaaCTG | 46F_47E_49D_50I_52G_53A_54P_55F_67L | 2.547803596 |
| 2406 | 1148 | TTCTTTTGTCCCATATGTGGGGCAAAGTTTactcagaaaggtaacctcctccgccacattaaaCTG | 46F_47F_49P_50I_52G_53A_54K_55F_67L | 2.546194319 |
| 2407 | 660 | TTCGACTGTCCTATTTGTGGGGCAGCCTTTactcagaaaggtaacctcctccgccacattaaaCTG | 46F_47D_49P_50I_52G_53A_54A_55F_67L | 2.540195995 |
| 2408 | 314 | TTCTGCTGTGAAATATGTGGGGCACGTTTTactcagaaaggtaacctcctccgccacattaaaCTG | 46F_47C_49E_50I_52G_53A_54R_55F_67L | 2.539525518 |
| 2409 | 2512 | TTCATGTGTGAAGTCTGTGGGGCACCCTTTactcagaaaggtaacctcctccgccacattaaaCTG | 46F_47M_49E_50V_52G_53A_54P_55F_67L | 2.538605377 |
| 2410 | 3714 | TTCACATGTGAGGTGTGTGGGGCAAGATTTactcagaaaggtaacctcctccgccacattaaaCTG | 46F_47T_49E_50V_52G_53A_54R_55F_67L | 2.537279719 |
| 2411 | 4245 | TTCTGGTGTAACGTGTGTGGGGCAGGCTTTactcagaaaggtaacctcctccgccacattaaaCTG | 46F_47W_49N_50V_52G_53A_54G_55F_67L | 2.534865194 |
| 2412 | 6756 | CTCAGCTGTCCTATTTGTGGATTTACCTGTactcagaaaggtaacctcctccgccacattaaaCTG | 46L_47S_49P_50I_52G_53F_54T_55C_67L | 2.532679515 |
| 2413 | 4114 | TTCTGGTGTGATCAATGTGGGGCACGCTTTactcagaaaggtaacctcctccgccacattaaaCTG | 46F_47W_49D_50Q_52G_53A_54R_55F_67L | 2.53263587 |
| 2414 | 7935 | TTAGTGTGCGAAATCTGCGGCTTCTCCTGCactcagaaaggtaacctcctccgccacattaaaCTG | 46L_47V_49E_50I_52G_53F_54S_55C_67L | 2.529458591 |
| 2415 | 1675 | TTCCATTGTCCAGTTTGTGGGGCATCATTTactcagaaaggtaacctcctccgccacattaaaCTG | 46F_47H_49P_50V_52G_53A_54S_55F_67L | 2.523095829 |
| 2416 | 1661 | TTCCACTGTCCTGTATGTGGGGCATGTTTTactcagaaaggtaacctcctccgccacattaaaCTG | 46F_47H_49P_50V_52G_53A_54C_55F_67L | 2.522456888 |
| 2417 | 4559 | TTCTACTGTCCTGTTTGTGGGGCATACTTTactcagaaaggtaacctcctccgccacattaaaCTG | 46F_47Y_49P_50V_52G_53A_54Y_55F_67L | 2.51737473 |
| 2418 | 6576 | CTGGGGTGTCCAATATGCGGATTTACCTGCactcagaaaggtaacctcctccgccacattaaaCTG | 46L_47G_49P_50I_52G_53F_54T_55C_67L | 2.51487154 |
| 2419 | 4535 | TTCTATTGTCCGCAGTGTGGGGCATCCTTTactcagaaaggtaacctcctccgccacattaaaCTG | 46F_47Y_49P_50Q_52G_53A_54S_55F_67L | 2.514087375 |
| 2420 | 4329 | TTCTATTGTGATATCTGTGGGGCACTATTTactcagaaaggtaacctcctccgccacattaaaCTG | 46F_47Y_49D_50I_52G_53A_54L_55F_67L | 2.512584874 |
| 2421 | 3705 | TTCACTTGTGAGGTGTGTGGGGCAGGTTTTactcagaaaggtaacctcctccgccacattaaaCTG | 46F_47T_49E_50V_52G_53A_54G_55F_67L | 2.509665724 |
| 2422 | 3376 | TTCTCGTGTGACATCTGTGGGGCAACTTTTactcagaaaggtaacctcctccgccacattaaaCTG | 46F_47S_49D_50I_52G_53A_54T_55F_67L | 2.507587195 |
| 2423 | 5675 | CTGTGGTGTAACCAGTGCGGCTTTTCCTGTactcagaaaggtaacctcctccgccacattaaaCTG | 46L_47W_49N_50Q_52G_53F_54S_55C_67L | 2.507341601 |
| 2424 | 2941 | TTCCAATGTGAAATCTGTGGGGCATGTTTTactcagaaaggtaacctcctccgccacattaaaCTG | 46F_47Q_49E_50I_52G_53A_54C_55F_67L | 2.506184846 |
| 2425 | 6476 | TTAGCCTGTCCCATTTGCGGGTTTACATGCactcagaaaggtaacctcctccgccacattaaaCTG | 46L_47A_49P_50I_52G_53F_54T_55C_67L | 2.503038819 |
| 2426 | 61 | TTCGCGTGTGAAATTTGTGGGGCATGTTTTactcagaaaggtaacctcctccgccacattaaaCTG | 46F_47A_49E_50I_52G_53A_54C_55F_67L | 2.49935546 |
| 2427 | 7895 | CTGAGTTGCGAAATCTGCGGCTTCAGCTGTactcagaaaggtaacctcctccgccacattaaaCTG | 46L_47S_49E_50I_52G_53F_54S_55C_67L | 2.497592842 |
| 2428 | 1008 | TTCTTCTGTGATGTGTGTGGGGCAAAATTTactcagaaaggtaacctcctccgccacattaaaCTG | 46F_47F_49D_50V_52G_53A_54K_55F_67L | 2.496976545 |
| 2429 | 4060 | TTCGTGTGTCCTGTCTGTGGGGCAGCGTTTactcagaaaggtaacctcctccgccacattaaaCTG | 46F_47V_49P_50V_52G_53A_54A_55F_67L | 2.496278861 |
| 2430 | 356 | TTCTGCTGTGAAGTGTGTGGGGCAACCTTTactcagaaaggtaacctcctccgccacattaaaCTG | 46F_47C_49E_50V_52G_53A_54T_55F_67L | 2.495515166 |
| 2431 | 1150 | TTCTTCTGTCCCATTTGTGGGGCAATGTTTactcagaaaggtaacctcctccgccacattaaaCTG | 46F_47F_49P_50I_52G_53A_54M_55F_67L | 2.493984602 |
| 2432 | 360 | TTCTGTTGTAATATCTGTGGGGCAGCCTTTactcagaaaggtaacctcctccgccacattaaaCTG | 46F_47C_49N_50I_52G_53A_54A_55F_67L | 2.480832793 |
| 2433 | 735 | TTCGAATGTGATATTTGTGGGGCATCTTTTactcagaaaggtaacctcctccgccacattaaaCTG | 46F_47E_49D_50I_52G_53A_54S_55F_67L | 2.479341427 |
| 2434 | 4549 | TTCTATTGTCCGGTGTGTGGGGCATTGTTTactcagaaaggtaacctcctccgccacattaaaCTG | 46F_47Y_49P_50V_52G_53A_54L_55F_67L | 2.475827221 |
| 2435 | 4288 | TTCTGGTGTCCACAGTGTGGGGCAAAGTTTactcagaaaggtaacctcctccgccacattaaaCTG | 46F_47W_49P_50Q_52G_53A_54K_55F_67L | 2.470920506 |
| 2436 | 7755 | CTTATCTGTGAGATATGCGGATTTTCCTGCactcagaaaggtaacctcctccgccacattaaaCTG | 46L_47I_49E_50I_52G_53F_54S_55C_67L | 2.468773637 |
| 2437 | 1135 | TTCTTCTGTAACGTTTGTGGGGCAAGCTTTactcagaaaggtaacctcctccgccacattaaaCTG | 46F_47F_49N_50V_52G_53A_54S_55F_67L | 2.467159274 |
| 2438 | 3552 | TTCTCATGTCCCATCTGTGGGGCACCGTTTactcagaaaggtaacctcctccgccacattaaaCTG | 46F_47S_49P_50I_52G_53A_54P_55F_67L | 2.466600982 |
| 2439 | 494 | TTCGACTGTGATATCTGTGGGGCAAGATTTactcagaaaggtaacctcctccgccacattaaaCTG | 46F_47D_49D_50I_52G_53A_54R_55F_67L | 2.457859545 |
| 2440 | 7699 | TTATTCTGCGAAATCTGCGGTTTTTATTGCactcagaaaggtaacctcctccgccacattaaaCTG | 46L_47F_49E_50I_52G_53F_54Y_55C_67L | 2.457669132 |
| 2441 | 4236 | TTCTGGTGTAACCAGTGTGGGGCAACATTTactcagaaaggtaacctcctccgccacattaaaCTG | 46F_47W_49N_50Q_52G_53A_54T_55F_67L | 2.455930322 |
| 2442 | 6495 | CTCTGCTGCCCCATCTGTGGTTTCAGTTGCactcagaaaggtaacctcctccgccacattaaaCTG | 46L_47C_49P_50I_52G_53F_54S_55C_67L | 2.454124998 |
| 2443 | 4492 | TTCTACTGTAATGTATGTGGGGCACCGTTTactcagaaaggtaacctcctccgccacattaaaCTG | 46F_47Y_49N_50V_52G_53A_54P_55F_67L | 2.453772058 |
| 2444 | 2995 | TTCCAGTGTGAGGTCTGTGGGGCATCCTTTactcagaaaggtaacctcctccgccacattaaaCTG | 46F_47Q_49E_50V_52G_53A_54S_55F_67L | 2.450120236 |
| 2445 | 1732 | TTCATTTGTGATGTCTGTGGGGCACCCTTTactcagaaaggtaacctcctccgccacattaaaCTG | 46F_47I_49D_50V_52G_53A_54P_55F_67L | 2.448338817 |
| 2446 | 4496 | TTCTATTGTAACGTCTGTGGGGCAACCTTTactcagaaaggtaacctcctccgccacattaaaCTG | 46F_47Y_49N_50V_52G_53A_54T_55F_67L | 2.444396506 |
| 2447 | 420 | TTCTGCTGTCCTATCTGTGGGGCAGCCTTTactcagaaaggtaacctcctccgccacattaaaCTG | 46F_47C_49P_50I_52G_53A_54A_55F_67L | 2.443895961 |
| 2448 | 3375 | TTCTCGTGTGACATCTGTGGGGCAAGCTTTactcagaaaggtaacctcctccgccacattaaaCTG | 46F_47S_49D_50I_52G_53A_54S_55F_67L | 2.440914476 |
| 2449 | 1190 | TTCTTTTGTCCCGTGTGTGGGGCAATGTTTactcagaaaggtaacctcctccgccacattaaaCTG | 46F_47F_49P_50V_52G_53A_54M_55F_67L | 2.439866852 |
| 2450 | 1276 | TTCGGATGTGAGATCTGTGGGGCAACGTTTactcagaaaggtaacctcctccgccacattaaaCTG | 46F_47G_49E_50I_52G_53A_54T_55F_67L | 2.436762056 |
| 2451 | 4170 | TTCTGGTGTGAGCAGTGTGGGGCAATGTTTactcagaaaggtaacctcctccgccacattaaaCTG | 46F_47W_49E_50Q_52G_53A_54M_55F_67L | 2.436698816 |
| 2452 | 912 | TTCGAGTGTCCTATCTGTGGGGCACCTTTTactcagaaaggtaacctcctccgccacattaaaCTG | 46F_47E_49P_50I_52G_53A_54P_55F_67L | 2.435210115 |
| 2453 | 3555 | TTCAGTTGTCCTATCTGTGGGGCAAGCTTTactcagaaaggtaacctcctccgccacattaaaCTG | 46F_47S_49P_50I_52G_53A_54S_55F_67L | 2.43323189 |
| 2454 | 1420 | TTCGGTTGTCCGGTCTGTGGGGCAGCCTTTactcagaaaggtaacctcctccgccacattaaaCTG | 46F_47G_49P_50V_52G_53A_54A_55F_67L | 2.432657187 |
| 2455 | 3060 | TTCCAATGTCCTATATGTGGGGCAGCTTTTactcagaaaggtaacctcctccgccacattaaaCTG | 46F_47Q_49P_50I_52G_53A_54A_55F_67L | 2.432491455 |
| 2456 | 6580 | CTGCACTGCCCAATCTGTGGATTCGCGTGCactcagaaaggtaacctcctccgccacattaaaCTG | 46L_47H_49P_50I_52G_53F_54A_55C_67L | 2.431664471 |
| 2457 | 3580 | TTCAGTTGTCCCGTCTGTGGGGCAGCCTTTactcagaaaggtaacctcctccgccacattaaaCTG | 46F_47S_49P_50V_52G_53A_54A_55F_67L | 2.430177552 |
| 2458 | 73 | TTCGCCTGTGAGATTTGTGGGGCACAATTTactcagaaaggtaacctcctccgccacattaaaCTG | 46F_47A_49E_50I_52G_53A_54Q_55F_67L | 2.428824136 |
| 2459 | 1560 | TTCCACTGTAATATTTGTGGGGCAGCCTTTactcagaaaggtaacctcctccgccacattaaaCTG | 46F_47H_49N_50I_52G_53A_54A_55F_67L | 2.425577066 |
| 2460 | 7965 | CTATACTGTGAAATCTGCGGATTCGGGTGCactcagaaaggtaacctcctccgccacattaaaCTG | 46L_47Y_49E_50I_52G_53F_54G_55C_67L | 2.425335678 |
| 2461 | 3674 | TTCACCTGTGAAATTTGTGGGGCAAGGTTTactcagaaaggtaacctcctccgccacattaaaCTG | 46F_47T_49E_50I_52G_53A_54R_55F_67L | 2.424726924 |
| 2462 | 4025 | TTCGTCTGTCCAATATGTGGGGCAGGCTTTactcagaaaggtaacctcctccgccacattaaaCTG | 46F_47V_49P_50I_52G_53A_54G_55F_67L | 2.421568645 |
| 2463 | 1753 | TTCATTTGTGAAATTTGTGGGGCACAATTTactcagaaaggtaacctcctccgccacattaaaCTG | 46F_47I_49E_50I_52G_53A_54Q_55F_67L | 2.420831881 |
| 2464 | 1261 | TTCGGCTGTGAAATCTGTGGGGCATGCTTTactcagaaaggtaacctcctccgccacattaaaCTG | 46F_47G_49E_50I_52G_53A_54C_55F_67L | 2.414115063 |
| 2465 | 3077 | TTCCAGTGTCCCATCTGTGGGGCAGTGTTTactcagaaaggtaacctcctccgccacattaaaCTG | 46F_47Q_49P_50I_52G_53A_54V_55F_67L | 2.413112964 |
| 2466 | 5676 | CTGTGGTGTAATCAATGTGGTTTTACGTGTactcagaaaggtaacctcctccgccacattaaaCTG | 46L_47W_49N_50Q_52G_53F_54T_55C_67L | 2.412921336 |
| 2467 | 240 | TTCTGCTGTGACATTTGTGGGGCAGCATTTactcagaaaggtaacctcctccgccacattaaaCTG | 46F_47C_49D_50I_52G_53A_54A_55F_67L | 2.41054515 |
| 2468 | 4448 | TTCTACTGTAATATCTGTGGGGCAAAGTTTactcagaaaggtaacctcctccgccacattaaaCTG | 46F_47Y_49N_50I_52G_53A_54K_55F_67L | 2.407709836 |
| 2469 | 1199 | TTCTTTTGTCCCGTGTGTGGGGCATATTTTactcagaaaggtaacctcctccgccacattaaaCTG | 46F_47F_49P_50V_52G_53A_54Y_55F_67L | 2.405700565 |
| 2470 | 1176 | TTCTTTTGTCCGCAATGTGGGGCAACCTTTactcagaaaggtaacctcctccgccacattaaaCTG | 46F_47F_49P_50Q_52G_53A_54T_55F_67L | 2.403359427 |
| 2471 | 3835 | TTCACCTGTCCAGTTTGTGGGGCAAGCTTTactcagaaaggtaacctcctccgccacattaaaCTG | 46F_47T_49P_50V_52G_53A_54S_55F_67L | 2.399128253 |
| 2472 | 3361 | TTCAGTTGTGATATATGTGGGGCATGTTTTactcagaaaggtaacctcctccgccacattaaaCTG | 46F_47S_49D_50I_52G_53A_54C_55F_67L | 2.397915027 |
| 2473 | 1097 | TTCTTTTGTAACATTTGTGGGGCAGTTTTTactcagaaaggtaacctcctccgccacattaaaCTG | 46F_47F_49N_50I_52G_53A_54V_55F_67L | 2.397522975 |
| 2474 | 180 | TTCGCATGTCCCATATGTGGGGCAGCTTTTactcagaaaggtaacctcctccgccacattaaaCTG | 46F_47A_49P_50I_52G_53A_54A_55F_67L | 2.394788912 |
| 2475 | 1275 | TTCGGATGTGAGATCTGTGGGGCATCCTTTactcagaaaggtaacctcctccgccacattaaaCTG | 46F_47G_49E_50I_52G_53A_54S_55F_67L | 2.390670261 |
| 2476 | 1510 | TTCCACTGTGAGATATGTGGGGCAATGTTTactcagaaaggtaacctcctccgccacattaaaCTG | 46F_47H_49E_50I_52G_53A_54M_55F_67L | 2.385985528 |
| 2477 | 354 | TTCTGTTGTGAGGTGTGTGGGGCACGTTTTactcagaaaggtaacctcctccgccacattaaaCTG | 46F_47C_49E_50V_52G_53A_54R_55F_67L | 2.383926788 |
| 2478 | 6760 | CTAACCTGCCCAATCTGCGGGTTCGCTTGTactcagaaaggtaacctcctccgccacattaaaCTG | 46L_47T_49P_50I_52G_53F_54A_55C_67L | 2.382203369 |
| 2479 | 1003 | TTCTTTTGTGATGTGTGTGGGGCAGAGTTTactcagaaaggtaacctcctccgccacattaaaCTG | 46F_47F_49D_50V_52G_53A_54E_55F_67L | 2.382063362 |
| 2480 | 1315 | TTCGGCTGTGAAGTTTGTGGGGCAAGTTTTactcagaaaggtaacctcctccgccacattaaaCTG | 46F_47G_49E_50V_52G_53A_54S_55F_67L | 2.381515235 |
| 2481 | 4339 | TTCTACTGTGATATCTGTGGGGCATACTTTactcagaaaggtaacctcctccgccacattaaaCTG | 46F_47Y_49D_50I_52G_53A_54Y_55F_67L | 2.380608841 |
| 2482 | 1441 | TTCCATTGTGACATTTGTGGGGCATGCTTTactcagaaaggtaacctcctccgccacattaaaCTG | 46F_47H_49D_50I_52G_53A_54C_55F_67L | 2.380531184 |
| 2483 | 3716 | TTCACCTGTGAAGTTTGTGGGGCAACCTTTactcagaaaggtaacctcctccgccacattaaaCTG | 46F_47T_49E_50V_52G_53A_54T_55F_67L | 2.37773251 |
| 2484 | 6593 | TTGCACTGCCCTATCTGTGGATTTCAATGCactcagaaaggtaacctcctccgccacattaaaCTG | 46L_47H_49P_50I_52G_53F_54Q_55C_67L | 2.377257477 |
| 2485 | 700 | TTCGACTGTCCGGTCTGTGGGGCAGCATTTactcagaaaggtaacctcctccgccacattaaaCTG | 46F_47D_49P_50V_52G_53A_54A_55F_67L | 2.375808557 |
| 2486 | 4309 | TTCTGGTGTCCAGTATGTGGGGCACTGTTTactcagaaaggtaacctcctccgccacattaaaCTG | 46F_47W_49P_50V_52G_53A_54L_55F_67L | 2.375623078 |
| 2487 | 3665 | TTCACGTGTGAGATATGTGGGGCAGGATTTactcagaaaggtaacctcctccgccacattaaaCTG | 46F_47T_49E_50I_52G_53A_54G_55F_67L | 2.368161261 |
| 2488 | 675 | TTCGATTGTCCTATTTGTGGGGCATCCTTTactcagaaaggtaacctcctccgccacattaaaCTG | 46F_47D_49P_50I_52G_53A_54S_55F_67L | 2.367929931 |
| 2489 | 1187 | TTCTTTTGTCCTGTCTGTGGGGCAATCTTTactcagaaaggtaacctcctccgccacattaaaCTG | 46F_47F_49P_50V_52G_53A_54I_55F_67L | 2.367619415 |
| 2490 | 4533 | TTCTACTGTCCCCAGTGTGGGGCACAGTTTactcagaaaggtaacctcctccgccacattaaaCTG | 46F_47Y_49P_50Q_52G_53A_54Q_55F_67L | 2.367611324 |
| 2491 | 1277 | TTCGGATGTGAAATTTGTGGGGCAGTGTTTactcagaaaggtaacctcctccgccacattaaaCTG | 46F_47G_49E_50I_52G_53A_54V_55F_67L | 2.365824958 |
| 2492 | 7579 | TTTTGGTGCGAGATTTGCGGCAGATATTTCactcagaaaggtaacctcctccgccacattaaaCTG | 46F_47W_49E_50I_52G_53R_54Y_55F_67L | 2.364517976 |
| 2493 | 4459 | TTCTATTGTAACATCTGTGGGGCATATTTTactcagaaaggtaacctcctccgccacattaaaCTG | 46F_47Y_49N_50I_52G_53A_54Y_55F_67L | 2.354235572 |
| 2494 | 7569 | TTTTGGTGCGAGATCTGTGGCCGATTATTTactcagaaaggtaacctcctccgccacattaaaCTG | 46F_47W_49E_50I_52G_53R_54L_55F_67L | 2.353528444 |
| 2495 | 1780 | TTCATATGTGAGGTCTGTGGGGCAGCGTTTactcagaaaggtaacctcctccgccacattaaaCTG | 46F_47I_49E_50V_52G_53A_54A_55F_67L | 2.353224455 |
| 2496 | 1631 | TTCCATTGTCCAATTTGTGGGGCAAACTTTactcagaaaggtaacctcctccgccacattaaaCTG | 46F_47H_49P_50I_52G_53A_54N_55F_67L | 2.350158637 |
| 2497 | 3107 | TTCCAGTGTCCAGTGTGTGGGGCAATTTTTactcagaaaggtaacctcctccgccacattaaaCTG | 46F_47Q_49P_50V_52G_53A_54I_55F_67L | 2.350073241 |
| 2498 | 2532 | TTCATGTGTAATATTTGTGGGGCACCCTTTactcagaaaggtaacctcctccgccacattaaaCTG | 46F_47M_49N_50I_52G_53A_54P_55F_67L | 2.348259335 |
| 2499 | 1017 | TTCTTCTGTGATGTGTGTGGGGCAGTGTTTactcagaaaggtaacctcctccgccacattaaaCTG | 46F_47F_49D_50V_52G_53A_54V_55F_67L | 2.347154697 |
| 2500 | 4249 | TTCTGGTGTAATGTGTGTGGGGCACTGTTTactcagaaaggtaacctcctccgccacattaaaCTG | 46F_47W_49N_50V_52G_53A_54L_55F_67L | 2.344499612 |
| 2501 | 900 | TTCGAGTGTCCAATCTGTGGGGCAGCGTTTactcagaaaggtaacctcctccgccacattaaaCTG | 46F_47E_49P_50I_52G_53A_54A_55F_67L | 2.343688971 |
| 2502 | 6575 | CTTGGCTGCCCAATTTGCGGGTTCTCTTGTactcagaaaggtaacctcctccgccacattaaaCTG | 46L_47G_49P_50I_52G_53F_54S_55C_67L | 2.338491003 |
| 2503 | 6785 | TTGGTGTGCCCCATATGTGGGTTCGGGTGTactcagaaaggtaacctcctccgccacattaaaCTG | 46L_47V_49P_50I_52G_53F_54G_55C_67L | 2.334487434 |
| 2504 | 1145 | TTCTTTTGTCCCATCTGTGGGGCAGGATTTactcagaaaggtaacctcctccgccacattaaaCTG | 46F_47F_49P_50I_52G_53A_54G_55F_67L | 2.334435924 |
| 2505 | 1556 | TTCCATTGTGAAGTGTGTGGGGCAACTTTTactcagaaaggtaacctcctccgccacattaaaCTG | 46F_47H_49E_50V_52G_53A_54T_55F_67L | 2.329605187 |
| 2506 | 2872 | TTCAATTGTCCAGTGTGTGGGGCACCCTTTactcagaaaggtaacctcctccgccacattaaaCTG | 46F_47N_49P_50V_52G_53A_54P_55F_67L | 2.328959964 |
| 2507 | 760 | TTCGAGTGTGATGTGTGTGGGGCAGCATTTactcagaaaggtaacctcctccgccacattaaaCTG | 46F_47E_49D_50V_52G_53A_54A_55F_67L | 2.328514371 |
| 2508 | 855 | TTCGAATGTAACATTTGTGGGGCATCATTTactcagaaaggtaacctcctccgccacattaaaCTG | 46F_47E_49N_50I_52G_53A_54S_55F_67L | 2.328479507 |
| 2509 | 255 | TTCTGTTGTGACATCTGTGGGGCATCTTTTactcagaaaggtaacctcctccgccacattaaaCTG | 46F_47C_49D_50I_52G_53A_54S_55F_67L | 2.327180743 |
| 2510 | 4234 | TTCTGGTGTAACCAGTGTGGGGCAAGATTTactcagaaaggtaacctcctccgccacattaaaCTG | 46F_47W_49N_50Q_52G_53A_54R_55F_67L | 2.326918154 |
| 2511 | 2981 | TTCCAGTGTGAAGTGTGTGGGGCATGCTTTactcagaaaggtaacctcctccgccacattaaaCTG | 46F_47Q_49E_50V_52G_53A_54C_55F_67L | 2.326517405 |
| 2512 | 1024 | TTCTTTTGTGAAATTTGTGGGGCATTCTTTactcagaaaggtaacctcctccgccacattaaaCTG | 46F_47F_49E_50I_52G_53A_54F_55F_67L | 2.32637088 |
| 2513 | 1012 | TTCTTCTGTGATGTTTGTGGGGCACCTTTTactcagaaaggtaacctcctccgccacattaaaCTG | 46F_47F_49D_50V_52G_53A_54P_55F_67L | 2.326057588 |
| 2514 | 3780 | TTCACGTGTCCAATCTGTGGGGCAGCTTTTactcagaaaggtaacctcctccgccacattaaaCTG | 46F_47T_49P_50I_52G_53A_54A_55F_67L | 2.325940584 |
| 2515 | 4035 | TTCGTCTGTCCTATCTGTGGGGCAAGTTTTactcagaaaggtaacctcctccgccacattaaaCTG | 46F_47V_49P_50I_52G_53A_54S_55F_67L | 2.325318267 |
| 2516 | 550 | TTCGATTGTGAAATTTGTGGGGCAATGTTTactcagaaaggtaacctcctccgccacattaaaCTG | 46F_47D_49E_50I_52G_53A_54M_55F_67L | 2.324660677 |
| 2517 | 4082 | TTCTGGTGTGATATTTGTGGGGCAGACTTTactcagaaaggtaacctcctccgccacattaaaCTG | 46F_47W_49D_50I_52G_53A_54D_55F_67L | 2.315393016 |
| 2518 | 596 | TTCGACTGTGAAGTTTGTGGGGCAACCTTTactcagaaaggtaacctcctccgccacattaaaCTG | 46F_47D_49E_50V_52G_53A_54T_55F_67L | 2.315176142 |
| 2519 | 301 | TTCTGCTGTGAAATCTGTGGGGCATGTTTTactcagaaaggtaacctcctcogccacattaaaCTG | 46F_47C_49E_50I_52G_53A_54C_55F_67L | 2.314444423 |
| 2520 | 4244 | TTCTGGTGTAACGTGTGTGGGGCATTCTTTactcagaaaggtaacctcctccgccacattaaaCTG | 46F_47W_49N_50V_52G_53A_54F_55F_67L | 2.312847552 |
| 2521 | 1517 | TTCCACTGTGAAATCTGTGGGGCAGTGTTTactcagaaaggtaacctcctccgccacattaaaCTG | 46F_47H_49E_50I_52G_53A_54V_55F_67L | 2.312148846 |
| 2522 | 799 | TTCGAATGTGAAATTTGTGGGGCATACTTTactcagaaaggtaacctcctccgccacattaaaCTG | 46F_47E_49E_50I_52G_53A_54Y_55F_67L | 2.30891618 |
| 2523 | 4531 | TTCTATTGTCCACAATGTGGGGCAAATTTTactcagaaaggtaacctcctccgccacattaaaCTG | 46F_47Y_49P_50Q_52G_53A_54N_55F_67L | 2.305420205 |
| 2524 | 581 | TTCGATTGTGAGGTATGTGGGGCATGTTTTactcagaaaggtaacctcctccgccacattaaaCTG | 46F_47D_49E_50V_52G_53A_54C_55F_67L | 2.304671993 |
| 2525 | 3915 | TTCGTGTGTGAAATCTGTGGGGCAAGCTTTactcagaaaggtaacctcctccgccacattaaaCTG | 46F_47V_49E_50I_52G_53A_54S_55F_67L | 2.30415063 |
| 2526 | 7675 | TTGGAGTGTGAGATTTGCGGGTTTTCCTGCactcagaaaggtaacctcctccgccacattaaaCTG | 46L_47E_49E_50I_52G_53F_54S_55C_67L | 2.302765246 |
| 2527 | 585 | TTCGATTGTGAGGTGTGTGGGGCAGGATTTactcagaaaggtaacctcctccgccacattaaaCTG | 46F_47D_49E_50V_52G_53A_54G_55F_67L | 2.299382208 |
| 2528 | 1062 | TTCTTCTGTGAGGTGTGTGGGGCAGACTTTactcagaaaggtaacctcctccgccacattaaaCTG | 46F_47F_49E_50V_52G_53A_54D_55F_67L | 2.299366473 |
| 2529 | 1554 | TTCCACTGTGAGGTTTGTGGGGCACGCTTTactcagaaaggtaacctcctccgccacattaaaCTG | 46F_47H_49E_50V_52G_53A_54R_55F_67L | 2.296296135 |
| 2530 | 4486 | TTCTACTGTAATGTGTGTGGGGCACACTTTactcagaaaggtaacctcctccgccacattaaaCTG | 46F_47Y_49N_50V_52G_53A_54H_55F_67L | 2.290359142 |
| 2531 | 481 | TTCGATTGTGACATATGTGGGGCATGCTTTactcagaaaggtaacctcctccgccacattaaaCTG | 46F_47D_49D_50I_52G_53A_54C_55F_67L | 2.289842504 |
| 2532 | 7691 | CTATTCTGCGAGATCTGTGGGTTCAATTGCactcagaaaggtaacctcctccgccacattaaaCTG | 46L_47F_49E_50I_52G_53F_54N_55C_67L | 2.289123388 |
| 2533 | 192 | TTCGCTTGTCCCATTTGTGGGGCACCATTTactcagaaaggtaacctcctccgccacattaaaCTG | 46F_47A_49P_50I_52G_53A_54P_55F_67L | 2.286959683 |
| 2534 | 77 | TTCGCGTGTGAGATCTGTGGGGCAGTGTTTactcagaaaggtaacctcctccgccacattaaaCTG | 46F_47A_49E_50I_52G_53A_54V_55F_67L | 2.285183688 |
| 2535 | 1188 | TTCTTCTGTCCCGTGTGTGGGGCAAAGTTTactcagaaaggtaacctcctccgccacattaaaCTG | 46F_47F_49P_50V_52G_53A_54K_55F_67L | 2.284596637 |
| 2536 | 2880 | TTCCAATGTGATATTTGTGGGGCAGCCTTTactcagaaaggtaacctcctccgccacattaaaCTG | 46F_47Q_49D_50I_52G_53A_54A_55F_67L | 2.282958949 |
| 2537 | 7570 | TTTTGGTGTGAGATATGCGGACGCATGTTCactcagaaaggtaacctcctccgccacattaaaCTG | 46F_47W_49E_50I_52G_53R_54M_55F_67L | 2.279865852 |
| 2538 | 1055 | TTCTTTTGTGAACAGTGTGGGGCAAGTTTTactcagaaaggtaacctcctccgccacattaaaCTG | 46F_47F_49E_50Q_52G_53A_54S_55F_67L | 2.278886239 |
| 2539 | 1041 | TTCTTCTGTGAACAGTGTGGGGCATGTTTTactcagaaaggtaacctcctccgccacattaaaCTG | 46F_47F_49E_50Q_52G_53A_54C_55F_67L | 2.275802837 |
| 2540 | 341 | TTCTGCTGTGAAGTATGTGGGGCATGTTTTactcagaaaggtaacctcctccgccacattaaaCTG | 46F_47C_49E_50V_52G_53A_54C_55F_67L | 2.273566305 |
| 2541 | 965 | TTCTTTTGTGACATATGTGGGGCAGGTTTTactcagaaaggtaacctcctccgccacattaaaCTG | 46F_47F_49D_50I_52G_53A_54G_55F_67L | 2.273231627 |
| 2542 | 7575 | TTTTGGTGTGAGATCTGCGGCCGCAGCTTCactcagaaaggtaacctcctccgccacattaaaCTG | 46F_47W_49E_50I_52G_53R_54S_55F_67L | 2.271545334 |
| 2543 | 7949 | TTGTGGTGCGAGATTTGTGGATTTCTGTGTactcagaaaggtaacctcctccgccacattaaaCTG | 46L_47W_49E_50I_52G_53F_54L_55C_67L | 2.268991165 |
| 2544 | 3117 | TTCCAATGTCCCGTATGTGGGGCAGTTTTTactcagaaaggtaacctcctccgccacattaaaCTG | 46F_47Q_49P_50V_52G_53A_54V_55F_67L | 2.266356131 |
| 2545 | 7561 | TTTTGGTGCGAGATTTGTGGCCGTTGCTTTactcagaaaggtaacctcctccgccacattaaaCTG | 46F_47W_49E_50I_52G_53R_54C_55F_67L | 2.26223011 |
| 2546 | 2712 | TTCAATTGTGAAATATGTGGGGCACCTTTTactcagaaaggtaacctcctccgccacattaaaCTG | 46F_47N_49E_50I_52G_53A_54P_55F_67L | 2.259614859 |
| 2547 | 4020 | TTCGTGTGTCCGATCTGTGGGGCAGCGTTTactcagaaaggtaacctcctccgccacattaaaCTG | 46F_47V_49P_50I_52G_53A_54A_55F_67L | 2.259251353 |
| 2548 | 1573 | TTCCACTGTAACATTTGTGGGGCACAATTTactcagaaaggtaacctcctccgccacattaaaCTG | 46F_47H_49N_50I_52G_53A_54Q_55F_67L | 2.257430009 |
| 2549 | 3945 | TTCGTGTGTGAAGTCTGTGGGGCAGGCTTTactcagaaaggtaacctcctccgccacattaaaCTG | 46F_47V_49E_50V_52G_53A_54G_55F_67L | 2.25486065 |
| 2550 | 6496 | TTGTGCTGTCCGATATGTGGATTCACCTGTactcagaaaggtaacctcctccgccacattaaaCTG | 46L_47C_49P_50I_52G_53F_54T_55C_67L | 2.254800255 |
| 2551 | 4297 | TTCTGGTGTCCTCAGTGTGGGGCAGTCTTTactcagaaaggtaacctcctccgccacattaaaCTG | 46F_47W_49P_50Q_52G_53A_54V_55F_67L | 2.25328262 |
| 2552 | 3061 | TTCCAGTGTCCTATCTGTGGGGCATGTTTTactcagaaaggtaacctcctccgccacattaaaCTG | 46F_47Q_49P_50I_52G_53A_54C_55F_67L | 2.253178755 |
| 2553 | 1785 | TTCATCTGTGAGGTGTGTGGGGCAGGCTTTactcagaaaggtaacctcctccgccacattaaaCTG | 46F_47I_49E_50V_52G_53A_54G_55F_67L | 2.251335024 |
| 2554 | 6818 | CTGTGGTGTCCCATTTGTGGTTTCTGGTGCactcagaaaggtaacctcctccgccacattaaaCTG | 46L_47W_49P_50I_52G_53F_54W_55C_67L | 2.251291041 |
| 2555 | 7958 | CTATGGTGCGAAATATGTGGCTTCTGGTGCactcagaaaggtaacctcctccgccacattaaaCTG | 46L_47W_49E_50I_52G_53F_54W_55C_67L | 2.249601629 |
| 2556 | 6352 | TTCAGGTGTCCAATATGTGGGCGGCCGTTCactcagaaaggtaacctcctccgccacattaaaCTG | 46F_47R_49P_50I_52G_53R_54P_55F_67L | 2.249319957 |
| 2557 | 4411 | TTCTACTGTGAGCAGTGTGGGGCAAACTTTactcagaaaggtaacctcctccgccacattaaaCTG | 46F_47Y_49E_50Q_52G_53A_54N_55F_67L | 2.248895859 |
| 2558 | 4065 | TTCGTGTGTCCCGTCTGTGGGGCAGGATTTactcagaaaggtaacctcctccgccacattaaaCTG | 46F_47V_49P_50V_52G_53A_54G_55F_67L | 2.246105734 |
| 2559 | 4198 | TTCTGGTGTGAAGTGTGTGGGGCATGGTTTactcagaaaggtaacctcctccgccacattaaaCTG | 46F_47W_49E_50V_52G_53A_54W_55F_67L | 2.245391883 |
| 2560 | 1395 | TTCGGCTGTCCCATCTGTGGGGCATCATTTactcagaaaggtaacctcctccgccacattaaaCTG | 46F_47G_49P_50I_52G_53A_54S_55F_67L | 2.243685556 |
| 2561 | 2956 | TTCCAATGTGAGATTTGTGGGGCAACATTTactcagaaaggtaacctcctccgccacattaaaCTG | 46F_47Q_49E_50I_52G_53A_54T_55F_67L | 2.24028872 |
| 2562 | 940 | TTCGAATGTCCTGTATGTGGGGCAGCATTTactcagaaaggtaacctcctccgccacattaaaCTG | 46F_47E_49P_50V_52G_53A_54A_55F_67L | 2.238842805 |
| 2563 | 6599 | CTTCATTGCCCCATCTGCGGGTTCTACTGCactcagaaaggtaacctcctccgccacattaaaCTG | 46L_47H_49P_50I_52G_53F_54Y_55C_67L | 2.236940904 |
| 2564 | 2997 | TTCCAGTGTGAGGTATGTGGGGCAGTGTTTactcagaaaggtaacctcctccgccacattaaaCTG | 46F_47Q_49E_50V_52G_53A_54V_55F_67L | 2.228798082 |
| 2565 | 4278 | TTCTGGTGTCCTATCTGTGGGGCATGGTTTactcagaaaggtaacctcctccgccacattaaaCTG | 46F_47W_49P_50I_52G_53A_54W_55F_67L | 2.226710614 |
| 2566 | 4108 | TTCTGGTGTGATCAGTGTGGGGCAAAATTTactcagaaaggtaacctcctccgccacattaaaCTG | 46F_47W_49D_50Q_52G_53A_54K_55F_67L | 2.225000232 |
| 2567 | 475 | TTCTGTTGTCCAGTCTGTGGGGCATCCTTTactcagaaaggtaacctcctccgccacattaaaCTG | 46F_47C_49P_50V_52G_53A_54S_55F_67L | 2.22493653 |
| 2568 | 6544 | CTGTTCTGCCCCATCTGCGGATTCTTTTGTactcagaaaggtaacctcctccgccacattaaaCTG | 46L_47F_49P_50I_52G_53F_54F_55C_67L | 2.223296971 |
| 2569 | 1480 | TTCCACTGTGACGTCTGTGGGGCAGCATTTactcagaaaggtaacctcctccgccacattaaaCTG | 46F_47H_49D_50V_52G_53A_54A_55F_67L | 2.222523249 |
| 2570 | 3615 | TTCACATGTGATATATGTGGGGCAAGCTTTactcagaaaggtaacctcctccgccacattaaaCTG | 46F_47T_49D_50I_52G_53A_54S_55F_67L | 2.222172391 |
| 2571 | 4505 | TTCTATTGTCCAATATGTGGGGCAGGTTTTactcagaaaggtaacctcctccgccacattaaaCTG | 46F_47Y_49P_50I_52G_53A_54G_55F_67L | 2.22111844 |
| 2572 | 1086 | TTCTTCTGTAATATATGTGGGGCACATTTTactcagaaaggtaacctcctccgccacattaaaCTG | 46F_47F_49N_50I_52G_53A_54H_55F_67L | 2.220654928 |
| 2573 | 1085 | TTCTTCTGTAATATTTGTGGGGCAGGGTTTactcagaaaggtaacctcctccgccacattaaaCTG | 46F_47F_49N_50I_52G_53A_54G_55F_67L | 2.219438445 |
| 2574 | 1561 | TTCCACTGTAATATCTGTGGGGCATGTTTTactcagaaaggtaacctcctccgccacattaaaCTG | 46F_47H_49N_50I_52G_53A_54C_55F_67L | 2.218800839 |
| 2575 | 3473 | TTCTCCTGTGAGGTGTGTGGGGCACAGTTTactcagaaaggtaacctcctccgccacattaaaCTG | 46F_47S_49E_50V_52G_53A_54Q_55F_67L | 2.217807837 |
| 2576 | 4167 | TTCTGGTGTGAGCAGTGTGGGGCAATCTTTactcagaaaggtaacctcctccgccacattaaaCTG | 46F_47W_49E_50Q_52G_53A_54I_55F_67L | 2.215702414 |
| 2577 | 1860 | TTCATTTGTCCTATATGTGGGGCAGCCTTTactcagaaaggtaacctcctccgccacattaaaCTG | 46F_47I_49P_50I_52G_53A_54A_55F_67L | 2.214274261 |
| 2578 | 2988 | TTCCAATGTGAGGTGTGTGGGGCAAAGTTTactcagaaaggtaacctcctccgccacattaaaCTG | 46F_47Q_49E_50V_52G_53A_54K_55F_67L | 2.21160683 |
| 2579 | 1006 | TTCTTTTGTGACGTGTGTGGGGCACATTTTactcagaaaggtaacctcctccgccacattaaaCTG | 46F_47F_49D_50V_52G_53A_54H_55F_67L | 2.208383429 |
| 2580 | 7577 | TTTTGGTGTGAGATCTGCGGCCGCGTCTTTactcagaaaggtaacctcctccgccacattaaaCTG | 46F_47W_49E_50I_52G_53R_54V_55F_67L | 2.208131169 |
| 2581 | 4228 | TTCTGGTGTAATCAATGTGGGGCAAAATTTactcagaaaggtaacctcctccgccacattaaaCTG | 46F_47W_49N_50Q_52G_53A_54K_55F_67L | 2.208097607 |
| 2582 | 3794 | TTCACGTGTCCCATATGTGGGGCACGCTTTactcagaaaggtaacctcctccgccacattaaaCTG | 46F_47T_49P_50I_52G_53A_54R_55F_67L | 2.206555347 |
| 2583 | 7684 | CTGTTTTGTGAAATCTGTGGCTTTTTCTGCactcagaaaggtaacctcctccgccacattaaaCTG | 46L_47F_49E_50I_52G_53F_54F_55C_67L | 2.20343033 |
| 2584 | 597 | TTCGACTGTGAGGTATGTGGGGCAGTCTTTactcagaaaggtaacctcctccgccacattaaaCTG | 46F_47D_49E_50V_52G_53A_54V_55F_67L | 2.202924215 |
| 2585 | 3640 | TTCACGTGTGACGTCTGTGGGGCAGCGTTTactcagaaaggtaacctcctccgccacattaaaCTG | 46F_47T_49D_50V_52G_53A_54A_55F_67L | 2.202099018 |
| 2586 | 6740 | CTTAGCTGCCCCATCTGCGGTTTCGCCTGCactcagaaaggtaacctcctccgccacattaaaCTG | 46L_47S_49P_50I_52G_53F_54A_55C_67L | 2.199244094 |
| 2587 | 4117 | TTCTGGTGTGATCAGTGTGGGGCAGTCTTTactcagaaaggtaacctcctccgccacattaaaCTG | 46F_47W_49D_50Q_52G_53A_54V_55F_67L | 2.193532868 |
| 2588 | 135 | TTCGCTTGTAATATTTGTGGGGCATCCTTTactcagaaaggtaacctcctccgccacattaaaCTG | 46F_47A_49N_50I_52G_53A_54S_55F_67L | 2.192954124 |
| 2589 | 220 | TTCGCCTGTCCTGTGTGTGGGGCAGCATTTactcagaaaggtaacctcctccgccacattaaaCTG | 46F_47A_49P_50V_52G_53A_54A_55F_67L | 2.192286079 |
| 2590 | 305 | TTCTGCTGTGAAATCTGTGGGGCAGGTTTTactcagaaaggtaacctcctccgccacattaaaCTG | 46F_47C_49E_50I_52G_53A_54G_55F_67L | 2.191398224 |
| 2591 | 4163 | TTCTGGTGTGAGCAGTGTGGGGCAGAGTTTactcagaaaggtaacctcctccgccacattaaaCTG | 46F_47W_49E_50Q_52G_53A_54E_55F_67L | 2.188692641 |
| 2592 | 1421 | TTCGGATGTCCCGTCTGTGGGGCATGCTTTactcagaaaggtaacctcctccgccacattaaaCTG | 46F_47G_49P_50V_52G_53A_54C_55F_67L | 2.187501421 |
| 2593 | 845 | TTCGAATGTAACATCTGTGGGGCAGGTTTTactcagaaaggtaacctcctccgccacattaaaCTG | 46F_47E_49N_50I_52G_53A_54G_55F_67L | 2.187064966 |
| 2594 | 372 | TTCTGTTGTAACATATGTGGGGCACCTTTTactcagaaaggtaacctcctccgccacattaaaCTG | 46F_47C_49N_50I_52G_53A_54P_55F_67L | 2.185830511 |
| 2595 | 1185 | TTCTTCTGTCCTGTATGTGGGGCAGGTTTTactcagaaaggtaacctcctccgccacattaaaCTG | 46F_47F_49P_50V_52G_53A_54G_55F_67L | 2.185481635 |
| 2596 | 3840 | TTCGTGTGTGACATCTGTGGGGCAGCCTTTactcagaaaggtaacctcctccgccacattaaaCTG | 46F_47V_49D_50I_52G_53A_54A_55F_67L | 2.185108998 |
| 2597 | 3400 | TTCTCATGTGACGTGTGTGGGGCAGCCTTTactcagaaaggtaacctcctccgccacattaaaCTG | 46F_47S_49D_50V_52G_53A_54A_55F_67L | 2.184961781 |
| 2598 | 4544 | TTCTACTGTCCCGTCTGTGGGGCATTCTTTactcagaaaggtaacctcctccgccacattaaaCTG | 46F_47Y_49P_50V_52G_53A_54F_55F_67L | 2.184026889 |
| 2599 | 1873 | TTCATTTGTCCAATTTGTGGGGCACAGTTTactcagaaaggtaacctcctccgccacattaaaCTG | 46F_47I_49P_50I_52G_53A_54Q_55F_67L | 2.182211205 |
| 2600 | 6460 | CTAGCTTGTCCAATCTGCGGTTTTGCATGTactcagaaaggtaacctcctccgccacattaaaCTG | 46L_47A_49P_50I_52G_53F_54A_55C_67L | 2.179193862 |
| 2601 | 7615 | CTGGCGTGTGAAATTTGTGGATTCAGTTGCactcagaaaggtaacctcctccgccacattaaaCTG | 46L_47A_49E_50I_52G_53F_54S_55C_67L | 2.178344908 |
| 2602 | 101 | TTCGCATGTGAGGTGTGTGGGGCATGCTTTactcagaaaggtaacctcctccgccacattaaaCTG | 46F_47A_49E_50V_52G_53A_54C_55F_67L | 2.177633792 |
| 2603 | 493 | TTCGACTGTGATATTTGTGGGGCACAGTTTactcagaaaggtaacctcctccgccacattaaaCTG | 46F_47D_49D_50I_52G_53A_54Q_55F_67L | 2.177348563 |
| 2604 | 4449 | TTCTACTGTAATATCTGTGGGGCACTCTTTactcagaaaggtaacctcctccgccacattaaaCTG | 46F_47Y_49N_50I_52G_53A_54L_55F_67L | 2.173219915 |
| 2605 | 715 | TTCGACTGTCCCGTCTGTGGGGCATCCTTTactcagaaaggtaacctcctccgccacattaaaCTG | 46F_47D_49P_50V_52G_53A_54S_55F_67L | 2.172157439 |
| 2606 | 3292 | TTCAGATGTAATGTTTGTGGGGCACCATTTactcagaaaggtaacctcctccgccacattaaaCTG | 46F_47R_49N_50V_52G_53A_54P_55F_67L | 2.171707964 |
| 2607 | 7916 | CTGACCTGCGAAATCTGCGGATTTACTTGCactcagaaaggtaacctcctccgccacattaaaCTG | 46L_47T_49E_50I_52G_53F_54T_55C_67L | 2.165191803 |
| 2608 | 1505 | TTCCATTGTGAAATCTGTGGGGCAGGCTTTactcagaaaggtaacctcctccgccacattaaaCTG | 46F_47H_49E_50I_52G_53A_54G_55F_67L | 2.164431616 |
| 2609 | 1636 | TTCCACTGTCCTATTTGTGGGGCAACCTTTactcagaaaggtaacctcctccgccacattaaaCTG | 46F_47H_49P_50I_52G_53A_54T_55F_67L | 2.16404143 |
| 2610 | 2948 | TTCCAGTGTGAAATTTGTGGGGCAAAGTTTactcagaaaggtaacctcctccgccacattaaaCTG | 46F_47Q_49E_50I_52G_53A_54K_55F_67L | 2.163501083 |
| 2611 | 1142 | TTCTTCTGTCCGATTTGTGGGGCAGATTTTactcagaaaggtaacctcctccgccacattaaaCTG | 46F_47F_49P_50I_52G_53A_54D_55F_67L | 2.163189939 |
| 2612 | 673 | TTCGATTGTCCCATCTGTGGGGCACAATTTactcagaaaggtaacctcctccgccacattaaaCTG | 46F_47D_49P_50I_52G_53A_54Q_55F_67L | 2.160412122 |
| 2613 | 116 | TTCGCATGTGAGGTTTGTGGGGCAACCTTTactcagaaaggtaacctcctccgccacattaaaCTG | 46F_47A_49E_50V_52G_53A_54T_55F_67L | 2.159467397 |
| 2614 | 4417 | TTCTACTGTGAACAATGTGGGGCAGTCTTTactcagaaaggtaacctcctccgccacattaaaCTG | 46F_47Y_49E_50Q_52G_53A_54V_55F_67L | 2.155569491 |
| 2615 | 1671 | TTCCATTGTCCCGTCTGTGGGGCAAACTTTactcagaaaggtaacctcctccgccacattaaaCTG | 46F_47H_49P_50V_52G_53A_54N_55F_67L | 2.155348428 |
| 2616 | 970 | TTCTTTTGTGACATCTGTGGGGCAATGTTTactcagaaaggtaacctcctccgccacattaaaCTG | 46F_47F_49D_50I_52G_53A_54M_55F_67L | 2.150761215 |
| 2617 | 674 | TTCGACTGTCCTATTTGTGGGGCAAGATTTactcagaaaggtaacctcctccgccacattaaaCTG | 46F_47D_49P_50I_52G_53A_54R_55F_67L | 2.149811903 |
| 2618 | 4545 | TTCTACTGTCCCGTCTGTGGGGCAGGATTTactcagaaaggtaacctcctccgccacattaaaCTG | 46F_47Y_49P_50V_52G_53A_54G_55F_67L | 2.14911469 |
| 2619 | 6590 | CTCCATTGTCCGATTTGTGGCTTCATGTGTactcagaaaggtaacctcctccgccacattaaaCTG | 46L_47H_49P_50I_52G_53F_54M_55C_67L | 2.146549469 |
| 2620 | 4521 | TTCTACTGTCCTCAGTGTGGGGCATGCTTTactcagaaaggtaacctcctccgccacattaaaCTG | 46F_47Y_49P_50Q_52G_53A_54C_55F_67L | 2.143554442 |
| 2621 | 4365 | TTCTACTGTGATGTCTGTGGGGCAGGTTTTactcagaaaggtaacctcctccgccacattaaaCTG | 46F_47Y_49D_50V_52G_53A_54G_55F_67L | 2.143245265 |
| 2622 | 460 | TTCTGTTGTCCTGTGTGTGGGGCAGCCTTTactcagaaaggtaacctcctccgccacattaaaCTG | 46F_47C_49P_50V_52G_53A_54A_55F_67L | 2.143060092 |
| 2623 | 72 | TTCGCATGTGAAATATGTGGGGCACCATTTactcagaaaggtaacctcctccgccacattaaaCTG | 46F_47A_49E_50I_52G_53A_54P_55F_67L | 2.140807136 |
| 2624 | 7567 | TTTTGGTGTGAGATTTGTGGGAGAATCTTCactcagaaaggtaacctcctccgccacattaaaCTG | 46F_47W_49E_50I_52G_53R_54I_55F_67L | 2.140122785 |
| 2625 | 1453 | TTCCACTGTGATATCTGTGGGGCACAGTTTactcagaaaggtaacctcctccgccacattaaaCTG | 46F_47H_49D_50I_52G_53A_54Q_55F_67L | 2.139314941 |
| 2626 | 3701 | TTCACTTGTGAGGTCTGTGGGGCATGTTTTactcagaaaggtaacctcctccgccacattaaaCTG | 46F_47T_49E_50V_52G_53A_54C_55F_67L | 2.136408018 |
| 2627 | 345 | TTCTGTTGTGAGGTCTGTGGGGCAGGCTTTactcagaaaggtaacctcctccgccacattaaaCTG | 46F_47C_49E_50V_52G_53A_54G_55F_67L | 2.136103561 |
| 2628 | 3676 | TTCACTTGTGAAATTTGTGGGGCAACCTTTactcagaaaggtaacctcctccgccacattaaaCTG | 46F_47T_49E_50I_52G_53A_54T_55F_67L | 2.134568282 |
| 2629 | 7635 | CTGTGTTGCGAGATTTGCGGCTTCAGTTGTactcagaaaggtaacctcctccgccacattaaaCTG | 46L_47C_49E_50I_52G_53F_54S_55C_67L | 2.132847756 |
| 2630 | 7571 | TTTTGGTGTGAAATTTGCGGTAGAAACTTCactcagaaaggtaacctcctccgccacattaaaCTG | 46F_47W_49E_50I_52G_53R_54N_55F_67L | 2.132535721 |
| 2631 | 1133 | TTCTTCTGTAATGTGTGTGGGGCACAGTTTactcagaaaggtaacctcctccgccacattaaaCTG | 46F_47F_49N_50V_52G_53A_54Q_55F_67L | 2.131438406 |
| 2632 | 1634 | TTCCACTGTCCTATTTGTGGGGCACGTTTTactcagaaaggtaacctcctccgccacattaaaCTG | 46F_47H_49P_50I_52G_53A_54R_55F_67L | 2.130598532 |
| 2633 | 4447 | TTCTATTGTAACATCTGTGGGGCAATCTTTactcagaaaggtaacctcctccgccacattaaaCTG | 46F_47Y_49N_50I_52G_53A_54I_55F_67L | 2.130032721 |
| 2634 | 2993 | TTCCAGTGTGAGGTTTGTGGGGCACAGTTTactcagaaaggtaacctcctccgccacattaaaCTG | 46F_47Q_49E_50V_52G_53A_54Q_55F_67L | 2.128153278 |
| 2635 | 317 | TTCTGTTGTGAGATTTGTGGGGCAGTGTTTactcagaaaggtaacctcctccgccacattaaaCTG | 46F_47C_49E_50I_52G_53A_54V_55F_67L | 2.126413284 |
| 2636 | 6584 | CTGCACTGCCCAATATGTGGCTTCTTTTGTactcagaaaggtaacctcctccgccacattaaaCTG | 46L_47H_49P_50I_52G_53F_54F_55C_67L | 2.125912337 |
| 2637 | 1120 | TTCTTTTGTAATGTTTGTGGGGCAGCCTTTactcagaaaggtaacctcctccgccacattaaaCTG | 46F_47F_49N_50V_52G_53A_54A_55F_67L | 2.125751632 |
| 2638 | 4327 | TTCTACTGTGACATTTGTGGGGCAATATTTactcagaaaggtaacctcctccgccacattaaaCTG | 46F_47Y_49D_50I_52G_53A_54I_55F_67L | 2.125526389 |
| 2639 | 3496 | TTCTCATGTAACATCTGTGGGGCAACTTTTactcagaaaggtaacctcctccgccacattaaaCTG | 46F_47S_49N_50I_52G_53A_54T_55F_67L | 2.124538265 |
| 2640 | 6615 | TTGATCTGCCCAATTTGCGGCTTCTCCTGCactcagaaaggtaacctcctccgccacattaaaCTG | 46L_47I_49P_50I_52G_53F_54S_55C_67L | 2.124489941 |
| 2641 | 3075 | TTCCAGTGTCCTATCTGTGGGGCAAGCTTTactcagaaaggtaacctcctccgccacattaaaCTG | 46F_47Q_49P_50I_52G_53A_54S_55F_67L | 2.121273836 |
| 2642 | 7560 | TTTTGGTGTGAGATTTGCGGAAGAGCTTTCactcagaaaggtaacctcctccgccacattaaaCTG | 46F_47W_49E_50I_52G_53R_54A_55F_67L | 2.11965447 |
| 2643 | 4363 | TTCTACTGTGACGTATGTGGGGCAGAGTTTactcagaaaggtaacctcctccgccacattaaaCTG | 46F_47Y_49D_50V_52G_53A_54E_55F_67L | 2.119366242 |
| 2644 | 3595 | TTCAGCTGTCCCGTTTGTGGGGCATCCTTTactcagaaaggtaacctcctccgccacattaaaCTG | 46F_47S_49P_50V_52G_53A_54S_55F_67L | 2.117878359 |
| 2645 | 2996 | TTCCAGTGTGAGGTCTGTGGGGCAACGTTTactcagaaaggtaacctcctccgccacattaaaCTG | 46F_47Q_49E_50V_52G_53A_54T_55F_67L | 2.114648194 |
| 2646 | 7576 | TTTTGGTGTGAGATTTGCGGCCGCACTTTCactcagaaaggtaacctcctccgccacattaaaCTG | 46F_47W_49E_50I_52G_53R_54T_55F_67L | 2.113553863 |
| 2647 | 1435 | TTCGGCTGTCCGGTGTGTGGGGCATCATTTactcagaaaggtaacctcctccgccacattaaaCTG | 46F_47G_49P_50V_52G_53A_54S_55F_67L | 2.110255902 |
| 2648 | 520 | TTCGACTGTGATGTCTGTGGGGCAGCGTTTactcagaaaggtaacctcctccgccacattaaaCTG | 46F_47D_49D_50V_52G_53A_54A_55F_67L | 2.10987256 |
| 2649 | 6586 | CTTCATTGCCCAATTTGCGGATTCCACTGTactcagaaaggtaacctcctccgccacattaaaCTG | 46L_47H_49P_50I_52G_53F_54H_55C_67L | 2.104440659 |
| 2650 | 3556 | TTCTCATGTCCCATCTGTGGGGCAACTTTTactcagaaaggtaacctcctccgccacattaaaCTG | 46F_47S_49P_50I_52G_53A_54T_55F_67L | 2.104235905 |
| 2651 | 0 | TTCGCCTGTGATATCTGTGGGGCAGCTTTTactcagaaaggtaacctcctccgccacattaaaCTG | 46F_47A_49D_50I_52G_53A_54A_55F_67L | 2.103578519 |
| 2652 | 781 | TTCGAGTGTGAAATCTGTGGGGCATGCTTTactcagaaaggtaacctcctccgccacattaaaCTG | 46F_47E_49E_50I_52G_53A_54C_55F_67L | 2.101851439 |
| 2653 | 3477 | TTCTCTTGTGAGGTTTGTGGGGCAGTGTTTactcagaaaggtaacctcctccgccacattaaaCTG | 46F_47S_49E_50V_52G_53A_54V_55F_67L | 2.101115742 |
| 2654 | 76 | TTCGCCTGTGAAATCTGTGGGGCAACATTTactcagaaaggtaacctcctccgccacattaaaCTG | 46F_47A_49E_50I_52G_53A_54T_55F_67L | 2.100337592 |
| 2655 | 7593 | TTTTATTGTGAGATCTGTGGCAGGCAATTTactcagaaaggtaacctcctccgccacattaaaCTG | 46F_47Y_49E_50I_52G_53R_54Q_55F_67L | 2.098348507 |
| 2656 | 614 | TTCGATTGTAACATCTGTGGGGCAAGGTTTactcagaaaggtaacctcctccgccacattaaaCTG | 46F_47D_49N_50I_52G_53A_54R_55F_67L | 2.097202365 |
| 2657 | 1053 | TTCTTCTGTGAGCAGTGTGGGGCACAATTTactcagaaaggtaacctcctccgccacattaaaCTG | 46F_47F_49E_50Q_52G_53A_54Q_55F_67L | 2.093002893 |
| 2658 | 7920 | TTAGTGTGCGAGATTTGTGGGTTCGCCTGCactcagaaaggtaacctcctccgccacattaaaCTG | 46L_47V_49E_50I_52G_53F_54A_55C_67L | 2.091899224 |
| 2659 | 1392 | TTCGGCTGTCCCATCTGTGGGGCACCTTTTactcagaaaggtaacctcctccgccacattaaaCTG | 46F_47G_49P_50I_52G_53A_54P_55F_67L | 2.091136414 |
| 2660 | 3834 | TTCACTTGTCCTGTTTGTGGGGCACGCTTTactcagaaaggtaacctcctccgccacattaaaCTG | 46F_47T_49P_50V_52G_53A_54R_55F_67L | 2.087950553 |
| 2661 | 1514 | TTCCATTGTGAAATATGTGGGGCACGATTTactcagaaaggtaacctcctccgccacattaaaCTG | 46F_47H_49E_50I_52G_53A_54R_55F_67L | 2.083026931 |
| 2662 | 7720 | TTGCATTGCGAGATTTGTGGATTTGCCTGCactcagaaaggtaacctcctccgccacattaaaCTG | 46L_47H_49E_50I_52G_53F_54A_55C_67L | 2.082532742 |
| 2663 | 1381 | TTCGGCTGTCCCATTTGTGGGGCATGTTTTactcagaaaggtaacctcctccgccacattaaaCTG | 46F_47G_49P_50I_52G_53A_54C_55F_67L | 2.080517078 |
| 2664 | 4502 | TTCTACTGTCCAATTTGTGGGGCAGATTTTactcagaaaggtaacctcctccgccacattaaaCTG | 46F_47Y_49P_50I_52G_53A_54D_55F_67L | 2.077229896 |
| 2665 | 1131 | TTCTTTTGTAATGTCTGTGGGGCAAACTTTactcagaaaggtaacctcctccgccacattaaaCTG | 46F_47F_49N_50V_52G_53A_54N_55F_67L | 2.075765123 |
| 2666 | 375 | TTCTGTTGTAACATTTGTGGGGCAAGCTTTactcagaaaggtaacctcctccgccacattaaaCTG | 46F_47C_49N_50I_52G_53A_54S_55F_67L | 2.073385056 |
| 2667 | 348 | TTCTGCTGTGAGGTCTGTGGGGCAAAATTTactcagaaaggtaacctcctccgccacattaaaCTG | 46F_47C_49E_50V_52G_53A_54K_55F_67L | 2.073153336 |
| 2668 | 3553 | TTCTCCTGTCCGATATGTGGGGCACAGTTTactcagaaaggtaacctcctccgccacattaaaCTG | 46F_47S_49P_50I_52G_53A_54Q_55F_67L | 2.071336036 |
| 2669 | 3661 | TTCACATGTGAAATTTGTGGGGCATGCTTTactcagaaaggtaacctcctccgccacattaaaCTG | 46F_47T_49E_50I_52G_53A_54C_55F_67L | 2.070661699 |
| 2670 | 1740 | TTCATTTGTGAAATCTGTGGGGCAGCATTTactcagaaaggtaacctcctccgccacattaaaCTG | 46F_47I_49E_50I_52G_53A_54A_55F_67L | 2.067571495 |
| 2671 | 7733 | TTACACTGCGAGATTTGTGGCTTCCAATGCactcagaaaggtaacctcctccgccacattaaaCTG | 46L_47H_49E_50I_52G_53F_54Q_55C_67L | 2.067055111 |
| 2672 | 1301 | TTCGGGTGTGAAGTTTGTGGGGCATGTTTTactcagaaaggtaacctcctccgccacattaaaCTG | 46F_47G_49E_50V_52G_53A_54C_55F_67L | 2.064573353 |
| 2673 | 1548 | TTCCATTGTGAAGTGTGTGGGGCAAAGTTTactcagaaaggtaacctcctccgccacattaaaCTG | 46F_47H_49E_50V_52G_53A_54K_55F_67L | 2.063031848 |
| 2674 | 7945 | CTGTGGTGCGAAATATGTGGCTTCGGGTGTactcagaaaggtaacctcctccgccacattaaaCTG | 46L_47W_49E_50I_52G_53F_54G_55C_67L | 2.05568346 |
| 2675 | 1492 | TTCCACTGTGATGTCTGTGGGGCACCATTTactcagaaaggtaacctcctccgccacattaaaCTG | 46F_47H_49D_50V_52G_53A_54P_55F_67L | 2.054378739 |
| 2676 | 3332 | TTCAGATGTCCACAGTGTGGGGCACCATTTactcagaaaggtaacctcctccgccacattaaaCTG | 46F_47R_49P_50Q_52G_53A_54P_55F_67L | 2.054024128 |
| 2677 | 7660 | CTGGAGTGCGAGATCTGCGGGTTTGCCTGCactcagaaaggtaacctcctccgccacattaaaCTG | 46L_47E_49E_50I_52G_53F_54A_55C_67L | 2.052189439 |
| 2678 | 2352 | TTCTTATGTCCCATCTGTGGGGCACCCTTTactcagaaaggtaacctcctccgccacattaaaCTG | 46F_47L_49P_50I_52G_53A_54P_55F_67L | 2.05169795 |
| 2679 | 1637 | TTCCACTGTCCCATCTGTGGGGCAGTGTTTactcagaaaggtaacctcctccgccacattaaaCTG | 46F_47H_49P_50I_52G_53A_54V_55F_67L | 2.050421931 |
| 2680 | 4075 | TTCGTCTGTCCTGTGTGTGGGGCATCTTTTactcagaaaggtaacctcctccgccacattaaaCTG | 46F_47V_49P_50V_52G_53A_54S_55F_67L | 2.049744317 |
| 2681 | 3434 | TTCTCGTGTGAAATTTGTGGGGCACGTTTTactcagaaaggtaacctcctccgccacattaaaCTG | 46F_47S_49E_50I_52G_53A_54R_55F_67L | 2.049523438 |
| 2682 | 4033 | TTCGTGTGTCCGATTTGTGGGGCACAGTTTactcagaaaggtaacctcctccgccacattaaaCTG | 46F_47V_49P_50I_52G_53A_54Q_55F_67L | 2.048375762 |
| 2683 | 6480 | CTGTGTTGCCCCATCTGTGGCTTTGCATGCactcagaaaggtaacctcctccgccacattaaaCTG | 46L_47C_49P_50I_52G_53F_54A_55C_67L | 2.048184526 |
| 2684 | 1144 | TTCTTCTGTCCTATTTGTGGGGCATTTTTTactcagaaaggtaacctcctccgccacattaaaCTG | 46F_47F_49P_50I_52G_53A_54F_55F_67L | 2.047276182 |
| 2685 | 3673 | TTCACCTGTGAGATTTGTGGGGCACAATTTactcagaaaggtaacctcctccgccacattaaaCTG | 46F_47T_49E_50I_52G_53A_54Q_55F_67L | 2.045437955 |
| 2686 | 6479 | CTTGCATGCCCGATCTGCGGCTTTTACTGTactcagaaaggtaacctcctccgccacattaaaCTG | 46L_47A_49P_50I_52G_53F_54Y_55C_67L | 2.042121212 |
| 2687 | 880 | TTCGAGTGTAATGTCTGTGGGGCAGCTTTTactcagaaaggtaacctcctccgccacattaaaCTG | 46F_47E_49N_50V_52G_53A_54A_55F_67L | 2.040716562 |
| 2688 | 2740 | TTCAATTGTGAAGTGTGTGGGGCAGCGTTTactcagaaaggtaacctcctccgccacattaaaCTG | 46F_47N_49E_50V_52G_53A_54A_55F_67L | 2.040118232 |
| 2689 | 583 | TTCGACTGTGAGGTCTGTGGGGCAGAATTTactcagaaaggtaacctcctccgccacattaaaCTG | 46F_47D_49E_50V_52G_53A_54E_55F_67L | 2.039267547 |
| 2690 | 7313 | TTCTTCTGCGAAATCTGCGGGAGGCAGTTTactcagaaaggtaacctcctccgccacattaaaCTG | 46F_47F_49E_50I_52G_53R_54Q_55F_67L | 2.035942149 |
| 2691 | 7900 | CTAACCTGCGAGATATGTGGGTTTGCATGCactcagaaaggtaacctcctccgccacattaaaCTG | 46L_47T_49E_50I_52G_53F_54A_55C_67L | 2.033703082 |
| 2692 | 4098 | TTCTGGTGTGATATATGTGGGGCATGGTTTactcagaaaggtaacctcctccgccacattaaaCTG | 46F_47W_49D_50I_52G_53A_54W_55F_67L | 2.032989961 |
| 2693 | 7587 | TTCTACTGCGAAATCTGCGGCCGGATTTTTactcagaaaggtaacctcctccgccacattaaaCTG | 46F_47Y_49E_50I_52G_53R_54I_55F_67L | 2.030549105 |
| 2694 | 661 | TTCGATTGTCCAATTTGTGGGGCATGTTTTactcagaaaggtaacctcctccgccacattaaaCTG | 46F_47D_49P_50I_52G_53A_54C_55F_67L | 2.028168225 |
| 2695 | 2932 | TTCCAATGTGACGTGTGTGGGGCACCCTTTactcagaaaggtaacctcctccgccacattaaaCTG | 46F_47Q_49D_50V_52G_53A_54P_55F_67L | 2.026676373 |
| 2696 | 3953 | TTCGTTTGTGAAGTGTGTGGGGCACAATTTactcagaaaggtaacctcctccgccacattaaaCTG | 46F_47V_49E_50V_52G_53A_54Q_55F_67L | 2.023424633 |
| 2697 | 4514 | TTCTATTGTCCGATCTGTGGGGCAAGATTTactcagaaaggtaacctcctccgccacattaaaCTG | 46F_47Y_49P_50I_52G_53A_54R_55F_67L | 2.022885642 |
| 2698 | 3781 | TTCACGTGTCCGATTTGTGGGGCATGTTTTactcagaaaggtaacctcctccgccacattaaaCTG | 46F_47T_49P_50I_52G_53A_54C_55F_67L | 2.021943272 |
| 2699 | 1189 | TTCTTCTGTCCTGTGTGTGGGGCACTCTTTactcagaaaggtaacctcctccgccacattaaaCTG | 46F_47F_49P_50V_52G_53A_54L_55F_67L | 2.020249123 |
| 2700 | 4554 | TTCTACTGTCCCGTGTGTGGGGCAAGGTTTactcagaaaggtaacctcctccgccacattaaaCTG | 46F_47Y_49P_50V_52G_53A_54R_55F_67L | 2.018410489 |
| 2701 | 1094 | TTCTTTTGTAATATATGTGGGGCAAGGTTTactcagaaaggtaacctcctccgccacattaaaCTG | 46F_47F_49N_50I_52G_53A_54R_55F_67L | 2.016983734 |
| 2702 | 1745 | TTCATTTGTGAAATCTGTGGGGCAGGGTTTactcagaaaggtaacctcctccgccacattaaaCTG | 46F_47I_49E_50I_52G_53A_54G_55F_67L | 2.014685324 |
| 2703 | 308 | TTCTGCTGTGAGATCTGTGGGGCAAAGTTTactcagaaaggtaacctcctccgccacattaaaCTG | 46F_47C_49E_50I_52G_53A_54K_55F_67L | 2.012750788 |
| 2704 | 3720 | TTCACTTGTAATATCTGTGGGGCAGCCTTTactcagaaaggtaacctcctccgccacattaaaCTG | 46F_47T_49N_50I_52G_53A_54A_55F_67L | 2.010963198 |
| 2705 | 6812 | CTATGGTGTCCCATCTGCGGTTTCCCTTGTactcagaaaggtaacctcctccgccacattaaaCTG | 46L_47W_49P_50I_52G_53F_54P_55C_67L | 2.010528771 |
| 2706 | 3913 | TTCGTGTGTGAGATCTGTGGGGCACAGTTTactcagaaaggtaacctcctccgccacattaaaCTG | 46F_47V_49E_50I_52G_53A_54Q_55F_67L | 2.007497724 |
| 2707 | 7716 | CTGGGGTGTGAGATCTGCGGGTTCACTTGCactcagaaaggtaacctcctccgccacattaaaCTG | 46L_47G_49E_50I_52G_53F_54T_55C_67L | 2.006165765 |
| 2708 | 4110 | TTCTGGTGTGACCAGTGTGGGGCAATGTTTactcagaaaggtaacctcctccgccacattaaaCTG | 46F_47W_49D_50Q_52G_53A_54M_55F_67L | 2.005474312 |
| 2709 | 3068 | TTCCAGTGTCCCATCTGTGGGGCAAAATTTactcagaaaggtaacctcctccgccacattaaaCTG | 46F_47Q_49P_50I_52G_53A_54K_55F_67L | 2.004971163 |
| 2710 | 1755 | TTCATATGTGAGATTTGTGGGGCATCTTTTactcagaaaggtaacctcctccgccacattaaaCTG | 46F_47I_49E_50I_52G_53A_54S_55F_67L | 2.002327988 |
| 2711 | 4031 | TTCGTCTGTCCAATCTGTGGGGCAAACTTTactcagaaaggtaacctcctccgccacattaaaCTG | 46F_47V_49P_50I_52G_53A_54N_55F_67L | 2.001433633 |

TABLE 4
A65 orthogonality; Estimated LogFC Coefficient calculated from the limma voom linear regression contrast of the positive vs negative read
counts for each individual mutant degron as defined relative to SEQ ID NO: 2.

| | | | Annotation of amino acids according | |
|---|---|---|---|---|
| | | | to their position in SEQ ID NO: 2, | |
| | | | wherein the stated one letter amino | |
| | | | acid may be the original amino acid | |
| | | DNA sequence encoding | found in SEQ ID NO: 2 or a | |
| | | positions F146-L167 of SEQ ID NO: 2 absent any substitutions | substitution (first numerical digit | |
| SEQ ID | Degron- | defined according to the encoding sequence below and the variant | dropped, i.e. position 146 | |
| NO | ID | amino acid sequence set out in column 4 of this Table | in SEQ ID NO: 2 = 46 as stated below) | A65_logFC |
| 2712 | 3232 | TTCAGGTGTGAGGTTTGTGGGGCACCGTTTactcagaaaggtaacctcctccgccacattaaaCTG | 46F_47R_49E_50V_52G_53A_54P_55F_67L | 3.39282224 |
| 2713 | 3132 | TTCAGGTGTGATATCTGTGGGGCACCCTTTactcagaaaggtaacctcctccgccacattaaaCTG | 46F_47R_49D_50I_52G_53A_54P_55F_67L | 3.322610123 |
| 2714 | 3192 | TTCCGCTGTGAAATCTGTGGGGCACCCTTTactcagaaaggtaacctcctccgccacattaaaCTG | 46F_47R_49E_50I_52G_53A_54P_55F_67L | 3.312302385 |
| 2715 | 3352 | TTCCGCTGTCCCGTTTGTGGGGCACCATTTactcagaaaggtaacctoctccgccacattaaaCTG | 46F_47R_49P_50V_52G_53A_54P_55F_67L | 3.268430718 |
| 2716 | 2532 | TTCATGTGTAATATTTGTGGGGCACCCTTTactcagaaaggtaacctcctccgccacattaaaCTG | 46F_47M_49N_50I_52G_53A_54P_55F_67L | 3.235780862 |
| 2717 | 3172 | TTCAGATGTGACGTTTGTGGGGCACCGTTTactcagaaaggtaacctcctccgccacattaaaCTG | 46F_47R_49D_50V_52G_53A_54P_55F_67L | 3.209511221 |
| 2718 | 2460 | TTCATGTGTGAGATCTGTGGGGCAGCTTTTactcagaaaggtaacctcctccgccacattaaaCTG | 46F_47M_49E_50I_52G_53A_54A_55F_67L | 3.194868019 |
| 2719 | 7976 | CTCTATTGCGAAATATGCGGTTTCACATGCactcagaaaggtaacctcctccgccacattaaaCTG | 46L_47Y_49E_50I_52G_53F_54T_55C_67L | 3.190510423 |
| 2720 | 6499 | CTATGCTGTCCTATTTGTGGTTTCTACTGTactcagaaaggtaacctcctccgccacattaaaCTG | 46L_47C_49P_50I_52G_53F_54Y_55C_67L | 3.159043039 |
| 2721 | 6496 | TTGTGCTGTCCGATATGTGGATTCACCTGTactcagaaaggtaacctcctccgccacattaaaCTG | 46L_47C_49P_50I_52G_53F_54T_55C_67L | 3.141026409 |
| 2722 | 4393 | TTCTACTGTGAGATCTGTGGGGCACAATTTactcagaaaggtaacctcctccgccacattaaaCTG | 46F_47Y_49E_50I_52G_53A_54Q_55F_67L | 3.134568016 |
| 2723 | 2472 | TTCATGTGTGAGATTTGTGGGGCACCATTTactcagaaaggtaacctcctccgccacattaaaCTG | 46F_47M_49E_50I_52G_53A_54P_55F_67L | 3.13278528 |
| 2724 | 2512 | TTCATGTGTGAAGTCTGTGGGGCACCCTTTactcagaaaggtaacctcctccgccacattaaaCTG | 46F_47M_49E_50V_52G_53A_54P_55F_67L | 3.115664344 |
| 2725 | 1424 | TTCGGCTGTCCCGTGTGTGGGGCATTTTTTactcagaaaggtaacctcctccgccacattaaaCTG | 46F_47G_49P_50V_52G_53A_54F_55F_67L | 3.113954241 |
| 2726 | 4421 | TTCTACTGTGAGGTGTGTGGGGCATGCTTTactcagaaaggtaacctcctccgccacattaaaCTG | 46F_47Y_49E_50V_52G_53A_54C_55F_67L | 3.104881583 |
| 2727 | 7975 | CTCTACTGCGAGATTTGCGGGTTTAGCTGCactcagaaaggtaacctcctccgccacattaaaCTG | 46L_47Y_49E_50I_52G_53F_54S_55C_67L | 3.099686388 |
| 2728 | 6835 | TTGTACTGCCCTATATGTGGGTTCAGCTGCactcagaaaggtaacctcctccgccacattaaaCTG | 46L_47Y_49P_50I_52G_53F_54S_55C_67L | 3.090231355 |
| 2729 | 2592 | TTCATGTGTCCCATATGTGGGGCACCCTTTactcagaaaggtaacctcctccgccacattaaaCTG | 46F_47M_49P_50I_52G_53A_54P_55F_67L | 3.089576647 |
| 2730 | 2632 | TTCATGTGTCCAGTTTGTGGGGCACCCTTTactcagaaaggtaacctcctccgccacattaaaCTG | 46F_47M_49P_50V_52G_53A_54P_55F_67L | 3.086942144 |
| 2731 | 2620 | TTCATGTGTCCCGTGTGTGGGGCAGCTTTTactcagaaaggtaacctcctccgccacattaaaCTG | 46F_47M_49P_50V_52G_53A_54A_55F_67L | 3.078320712 |
| 2732 | 7956 | CTCTGGTGTGAAATTTGCGGATTCACCTGCactcagaaaggtaacctcctccgccacattaaaCTG | 46L_47W_49E_50I_52G_53F_54T_55C_67L | 3.077619304 |
| 2733 | 7955 | CTGTGGTGCGAGATCTGCGGCTTCAGCTGTactcagaaaggtaacctcctccgccacattaaaCTG | 46L_47W_49E_50I_52G_53F_54S_55C_67L | 3.076869614 |
| 2734 | 7816 | CTCATGTGTGAGATATGTGGGTTTACATGTactcagaaaggtaacctcctccgccacattaaaCTG | 46L_47M_49E_50I_52G_53F_54T_55C_67L | 3.074033576 |
| 2735 | 7961 | TTGTACTGTGAGATCTGCGGATTCTGTTGTactcagaaaggtaacctcctccgccacattaaaCTG | 46L_47Y_49E_50I_52G_53F_54C_55C_67L | 3.065624674 |
| 2736 | 4392 | TTCTATTGTGAAATTTGTGGGGCACCATTTactcagaaaggtaacctcctccgccacattaaaCTG | 46F_47Y_49E_50I_52G_53A_54P_55F_67L | 3.063967823 |
| 2737 | 6486 | CTGTGCTGCCCGATCTGTGGGTTTCATTGCactcagaaaggtaacctcctccgccacattaaaCTG | 46L_47C_49P_50I_52G_53F_54H_55C_67L | 3.060372042 |
| 2738 | 1753 | TTCATTTGTGAAATTTGTGGGGCACAATTTactcagaaaggtaacctcctccgccacattaaaCTG | 46F_47I_49E_50I_52G_53A_54Q_55F_67L | 3.055225426 |
| 2739 | 3252 | TTCAGGTGTAACATTTGTGGGGCACCGTTTactcagaaaggtaacctcctccgccacattaaaCTG | 46F_47R_49N_50I_52G_53A_54P_55F_67L | 3.048990751 |
| 2740 | 6816 | CTGTGGTGTCCCATCTGCGGCTTCACTTGTactcagaaaggtaacctcctccgccacattaaaCTG | 46L_47W_49P_50I_52G_53F_54T_55C_67L | 3.048327024 |
| 2741 | 3312 | TTCCGCTGTCCCATCTGTGGGGCACCATTTactcagaaaggtaacctcctccgccacattaaaCTG | 46F_47R_49P_50I_52G_53A_54P_55F_67L | 3.044559924 |
| 2742 | 2412 | TTCATGTGTGACATTTGTGGGGCACCCTTTactcagaaaggtaacctcctccgccacattaaaCTG | 46F_47M_49D_50I_52G_53A_54P_55F_67L | 3.025817537 |
| 2743 | 6732 | CTGAGATGTCCTATTTGTGGCTTTCCCTGCactcagaaaggtaacctcctccgccacattaaaCTG | 46L_47R_49P_50I_52G_53F_54P_55C_67L | 3.024445694 |
| 2744 | 6495 | CTCTGCTGCCCCATCTGTGGTTTCAGTTGCactcagaaaggtaacctcctccgccacattaaaCTG | 46L_47C_49P_50I_52G_53F_54S_55C_67L | 3.004925159 |
| 2745 | 477 | TTCTGTTGTCCGGTGTGTGGGGCAGTCTTTactcagaaaggtaacctcctccgccacattaaaCTG | 46F_47C_49P_50V_52G_53A_54V_55F_67L | 3.002745118 |
| 2746 | 3580 | TTCAGTTGTCCCGTCTGTGGGGCAGCCTTTactcagaaaggtaacctcctccgccacattaaaCTG | 46F_47S_49P_50V_52G_53A_54A_55F_67L | 2.998405924 |
| 2747 | 4433 | TTCTATTGTGAGGTTTGTGGGGCACAATTTactcagaaaggtaacctcctccgccacattaaaCTG | 46F_47Y_49E_50V_52G_53A_54Q_55F_67L | 2.993685477 |
| 2748 | 2582 | TTCATGTGTCCCATCTGTGGGGCAGATTTTactcagaaaggtaacctcctccgccacattaaaCTG | 46F_47M_49P_50I_52G_53A_54D_55F_67L | 2.99259872 |
| 2749 | 1793 | TTCATTTGTGAGGTATGTGGGGCACAGTTTactcagaaaggtaacctcctccgccacattaaaCTG | 46F_47I_49E_50V_52G_53A_54Q_55F_67L | 2.991481394 |
| 2750 | 461 | TTCTGCTGTCCCGTGTGTGGGGCATGTTTTactcagaaaggtaacctcctccgccacattaaaCTG | 46F_47C_49P_50V_52G_53A_54C_55F_67L | 2.990320109 |
| 2751 | 6675 | CTAATGTGTCCAATTTGCGGTTTCAGCTGTactcagaaaggtaacctcctccgccacattaaaCTG | 46L_47M_49P_50I_52G_53F_54S_55C_67L | 2.98647291 |
| 2752 | 4420 | TTCTACTGTGAAGTCTGTGGGGCAGCTTTTactcagaaaggtaacctcctccgccacattaaaCTG | 46F_47Y_49E_50V_52G_53A_54A_55F_67L | 2.984779535 |
| 2753 | 6836 | CTCTATTGTCCCATTTGTGGGTTCACTTGCactcagaaaggtaacctcctccgccacattaaaCTG | 46L_47Y_49P_50I_52G_53F_54T_55C_67L | 2.972484201 |
| 2754 | 1420 | TTCGGTTGTCCGGTCTGTGGGGCAGCCTTTactcagaaaggtaacctcctccgccacattaaaCTG | 46F_47G_49P_50V_52G_53A_54A_55F_67L | 2.965578783 |
| 2755 | 4435 | TTCTACTGTGAAGTTTGTGGGGCAAGCTTTactcagaaaggtaacctcctccgccacattaaaCTG | 46F_47Y_49E_50V_52G_53A_54S_55F_67L | 2.941176449 |
| 2756 | 3292 | TTCAGATGTAATGTTTGTGGGGCACCATTTactcagaaaggtaacctcctccgccacattaaaCTG | 46F_47R_49N_50V_52G_53A_54P_55F_67L | 2.939043997 |
| 2757 | 2515 | TTCATGTGTGAAGTGTGTGGGGCATCGTTTactcagaaaggtaacctcctccgccacattaaaCTG | 46F_47M_49E_50V_52G_53A_54S_55F_67L | 2.938150988 |
| 2758 | 2580 | TTCATGTGTCCAATTTGTGGGGCAGCCTTTactcagaaaggtaacctcctccgccacattaaaCTG | 46F_47M_49P_50I_52G_53A_54A_55F_67L | 2.936538778 |
| 2759 | 2622 | TTCATGTGTCCTGTGTGTGGGGCAGATTTTactcagaaaggtaacctcctccgccacattaaaCTG | 46F_47M_49P_50V_52G_53A_54D_55F_67L | 2.928631849 |
| 2760 | 6779 | CTAACCTGTCCCATATGTGGTTTTTATTGCactcagaaaggtaacctcctccgccacattaaaCTG | 46L_47T_49P_50I_52G_53F_54Y_55C_67L | 2.927338274 |
| 2761 | 2392 | TTCCTGTGTCCCGTCTGTGGGGCACCTTTTactcagaaaggtaacctcctccgccacattaaaCTG | 46F_47L_49P_50V_52G_53A_54P_55F_67L | 2.926424217 |
| 2762 | 6801 | CTCTGGTGTCCCATTTGTGGCTTTTGCTGCactcagaaaggtaacctcctccgccacattaaaCTG | 46L_47W_49P_50I_52G_53F_54C_55C_67L | 2.924004769 |
| 2763 | 2631 | TTCATGTGTCCAGTGTGTGGGGCAAATTTTactcagaaaggtaacctcctccgccacattaaaCTG | 46F_47M_49P_50V_52G_53A_54N_55F_67L | 2.923330908 |
| 2764 | 3953 | TTCGTTTGTGAAGTGTGTGGGGCACAATTTactcagaaaggtaacctcctccgccacattaaaCTG | 46F_47V_49E_50V_52G_53A_54Q_55F_67L | 2.920131265 |
| 2765 | 6759 | CTGTCTTGTCCTATCTGTGGTTTCTATTGCactcagaaaggtaacctcctccgccacattaaaCTG | 46L_47S_49P_50I_52G_53F_54Y_55C_67L | 2.919961849 |
| 2766 | 4381 | TTCTATTGTGAGATATGTGGGGCATGCTTTactcagaaaggtaacctcctccgccacattaaaCTG | 46F_47Y_49E_50I_52G_53A_54C_55F_67L | 2.919796883 |
| 2767 | 911 | TTCGAATGTCCCATATGTGGGGCAAATTTTactcagaaaggtaacctcctccgccacattaaaCTG | 46F_47E_49P_50I_52G_53A_54N_55F_67L | 2.918489254 |
| 2768 | 4380 | TTCTATTGTGAAATCTGTGGGGCAGCCTTTactcagaaaggtaacctcctccgccacattaaaCTG | 46F_47Y_49E_50I_52G_53A_54A_55F_67L | 2.918312252 |
| 2769 | 6821 | TTGTATTGTCCCATTTGTGGCTTTTGTTGTactcagaaaggtaacctcctccgccacattaaaCTG | 46L_47Y_49P_50I_52G_53F_54C_55C_67L | 2.910428855 |
| 2770 | 4395 | TTCTATTGTGAGATCTGTGGGGCATCTTTTactcagaaaggtaacctcctccgccacattaaaCTG | 46F_47Y_49E_50I_52G_53A_54S_55F_67L | 2.906293591 |
| 2771 | 2595 | TTCATGTGTCCTATCTGTGGGGCATCTTTTactcagaaaggtaacctcctccgccacattaaaCTG | 46F_47M_49P_50I_52G_53A_54S_55F_67L | 2.906029822 |
| 2772 | 2475 | TTCATGTGTGAAATCTGTGGGGCATCATTTactcagaaaggtaacctcctccgccacattaaaCTG | 46F_47M_49E_50I_52G_53A_54S_55F_67L | 2.904813836 |
| 2773 | 2633 | TTCATGTGTCCAGTTTGTGGGGCACAATTTactcagaaaggtaacctcctccgccacattaaaCTG | 46F_47M_49P_50V_52G_53A_54Q_55F_67L | 2.895699062 |
| 2774 | 1913 | TTCATCTGTCCTGTGTGTGGGGCACAATTTactcagaaaggtaacctcctccgccacattaaaCTG | 46F_47I_49P_50V_52G_53A_54Q_55F_67L | 2.894482972 |
| 2775 | 6679 | CTTATGTGCCCCATTTGCGGATTTTACTGCactcagaaaggtaacctcctccgccacattaaaCTG | 46L_47M_49P_50I_52G_53F_54Y_55C_67L | 2.882826225 |
| 2776 | 1380 | TTCGGGTGTCCCATATGTGGGGCAGCTTTTactcagaaaggtaacctcctccgccacattaaaCTG | 46F_47G_49P_50I_52G_53A_54A_55F_67L | 2.882393915 |
| 2777 | 3795 | TTCACTTGTCCAATCTGTGGGGCAAGCTTTactcagaaaggtaacctcctccgccacattaaaCTG | 46F_47T_49P_50I_52G_53A_54S_55F_67L | 2.878380215 |
| 2778 | 1384 | TTCGGCTGTCCTATCTGTGGGGCATTTTTTactcagaaaggtaacctcctccgccacattaaaCTG | 46F_47G_49P_50I_52G_53A_54F_55F_67L | 2.878346729 |
| 2779 | 341 | TTCTGCTGTGAAGTATGTGGGGCATGTTTTactcagaaaggtaacctcctccgccacattaaaCTG | 46F_47C_49E_50V_52G_53A_54C_55F_67L | 2.877240806 |
| 2780 | 6775 | CTCACCTGCCCAATCTGCGGGTTCAGCTGTactcagaaaggtaacctcctccgccacattaaaCTG | 46L_47T_49P_50I_52G_53F_54S_55C_67L | 2.870701736 |
| 2781 | 2591 | TTCATGTGTCCCATCTGTGGGGCAAACTTTactcagaaaggtaacctcctccgccacattaaaCTG | 46F_47M_49P_50I_52G_53A_54N_55F_67L | 2.870606424 |
| 2782 | 433 | TTCTGCTGTCCTATTTGTGGGGCACAATTTactcagaaaggtaacctcctccgccacattaaaCTG | 46F_47C_49P_50I_52G_53A_54Q_55F_67L | 2.866766898 |
| 2783 | 1632 | TTCCACTGTCCAATTTGTGGGGCACCTTTTactcagaaaggtaacctcctccgccacattaaaCTG | 46F_47H_49P_50I_52G_53A_54P_55F_67L | 2.865865478 |
| 2784 | 3220 | TTCCGATGTGAAGTGTGTGGGGCAGCATTTactcagaaaggtaacctcctccgccacattaaaCTG | 46F_47R_49E_50V_52G_53A_54A_55F_67L | 2.863023751 |
| 2785 | 6819 | CTGTGGTGTCCCATCTGCGGCTTTTATTGCactcagaaaggtaacctcctccgccacattaaaCTG | 46L_47W_49P_50I_52G_53F_54Y_55C_67L | 2.860353926 |
| 2786 | 6660 | CTTATGTGTCCAATCTGCGGTTTTGCATGTactcagaaaggtaacctcctccgccacattaaaCTG | 46L_47M_49P_50I_52G_53F_54A_55C_67L | 2.860106014 |
| 2787 | 3913 | TTCGTGTGTGAGATCTGTGGGGCACAGTTTactcagaaaggtaacctcctccgccacattaaaCTG | 46F_47V_49E_50I_52G_53A_54Q_55F_67L | 2.859911713 |
| 2788 | 6810 | CTGTGGTGCCCCATTTGTGGCTTCATGTGTactcagaaaggtaacctcctccgccacattaaaCTG | 46L_47W_49P_50I_52G_53F_54M_55C_67L | 2.859378459 |
| 2789 | 6490 | CTATGCTGCCCAATATGCGGGTTTATGTGCactcagaaaggtaacctcctccgccacattaaaCTG | 46L_47C_49P_50I_52G_53F_54M_55C_67L | 2.858055513 |
| 2790 | 6599 | CTTCATTGCCCCATCTGCGGGTTCTACTGCactcagaaaggtaacctcctccgccacattaaaCTG | 46L_47H_49P_50I_52G_53F_54Y_55C_67L | 2.854839901 |
| 2791 | 473 | TTCTGCTGTCCCGTTTGTGGGGCACAATTTactcagaaaggtaacctcctccgccacattaaaCTG | 46F_47C_49P_50V_52G_53A_54Q_55F_67L | 2.842921363 |
| 2792 | 1873 | TTCATTTGTCCAATTTGTGGGGCACAGTTTactcagaaaggtaacctcctccgccacattaaaCTG | 46F_47I_49P_50I_52G_53A_54Q_55F_67L | 2.842608744 |
| 2793 | 3067 | TTCCAGTGTCCTATCTGTGGGGCAATTTTTactcagaaaggtaacctcctccgccacattaaaCTG | 46F_47Q_49P_50I_52G_53A_54I_55F_67L | 2.838399852 |
| 2794 | 1439 | TTCGGATGTCCCGTCTGTGGGGCATACTTTactcagaaaggtaacctcctccgccacattaaaCTG | 46F_47G_49P_50V_52G_53A_54Y_55F_67L | 2.836568683 |
| 2795 | 6815 | CTGTGGTGCCCGATCTGTGGATTTAGCTGCactcagaaaggtaacctcctccgccacattaaaCTG | 46L_47W_49P_50I_52G_53F_54S_55C_67L | 2.836175278 |
| 2796 | 4512 | TTCTATTGTCCAATATGTGGGGCACCGTTTactcagaaaggtaacctcctccgccacattaaaCTG | 46F_47Y_49P_50I_52G_53A_54P_55F_67L | 2.836118883 |
| 2797 | 2947 | TTCCAATGTGAGATCTGTGGGGCAATCTTTactcagaaaggtaacctcctccgccacattaaaCTG | 46F_47Q_49E_50I_52G_53A_54I_55F_67L | 2.835902449 |
| 2798 | 3839 | TTCACATGTCCTGTGTGTGGGGCATACTTTactcagaaaggtaacctcctccgccacattaaaCTG | 46F_47T_49P_50V_52G_53A_54Y_55F_67L | 2.834947057 |
| 2799 | 6595 | TTACATTGCCCAATATGTGGCTTCTCCTGTactcagaaaggtaacctcctccgccacattaaaCTG | 46L_47H_49P_50I_52G_53F_54S_55C_67L | 2.831623019 |
| 2800 | 3799 | TTCACTTGTCCAATCTGTGGGGCATATTTTactcagaaaggtaacctcctccgccacattaaaCTG | 46F_47T_49P_50I_52G_53A_54Y_55F_67L | 2.829249065 |
| 2801 | 1399 | TTCGGCTGTCCCATCTGTGGGGCATATTTTactcagaaaggtaacctcctccgccacattaaaCTG | 46F_47G_49P_50I_52G_53A_54Y_55F_67L | 2.827879511 |
| 2802 | 4073 | TTCGTCTGTCCTGTGTGTGGGGCACAGTTTactcagaaaggtaacctcctccgccacattaaaCTG | 46F_47V_49P_50V_52G_53A_54Q_55F_67L | 2.825227498 |
| 2803 | 4193 | TTCTGGTGTGAGGTGTGTGGGGCACAGTTTactcagaaaggtaacctcctccgccacattaaaCTG | 46F_47W_49E_50V_52G_53A_54Q_55F_67L | 2.824383174 |
| 2804 | 421 | TTCTGTTGTCCAATTTGTGGGGCATGCTTTactcagaaaggtaacctcctccgccacattaaaCTG | 46F_47C_49P_50I_52G_53A_54C_55F_67L | 2.822694743 |
| 2805 | 4437 | TTCTACTGTGAGGTGTGTGGGGCAGTGTTTactcagaaaggtaacctcctccgccacattaaaCTG | 46F_47Y_49E_50V_52G_53A_54V_55F_67L | 2.821551146 |
| 2806 | 1397 | TTCGGGTGTCCTATCTGTGGGGCAGTGTTTactcagaaaggtaacctcctccgccacattaaaCTG | 46F_47G_49P_50I_52G_53A_54V_55F_67L | 2.81816629 |
| 2807 | 4552 | TTCTATTGTCCAGTGTGTGGGGCACCTTTTactcagaaaggtaacctcctccgccacattaaaCTG | 46F_47Y_49P_50V_52G_53A_54P_55F_67L | 2.818068918 |
| 2808 | 3781 | TTCACGTGTCCGATTTGTGGGGCATGTTTTactcagaaaggtaacctcctccgccacattaaaCTG | 46F_47T_49P_50I_52G_53A_54C_55F_67L | 2.814779263 |
| 2809 | 1421 | TTCGGATGTCCCGTCTGTGGGGCATGCTTTactcagaaaggtaacctcctccgccacattaaaCTG | 46F_47G_49P_50V_52G_53A_54C_55F_67L | 2.814714234 |
| 2810 | 6804 | CTATGGTGTCCAATCTGCGGCTTTTTTTGCactcagaaaggtaacctcctccgccacattaaaCTG | 46L_47W_49P_50I_52G_53F_54F_55C_67L | 2.814402805 |
| 2811 | 6813 | CTGTGGTGTCCGATCTGCGGCTTTCAATGCactcagaaaggtaacctcctccgccacattaaaCTG | 46L_47W_49P_50I_52G_53F_54Q_55C_67L | 2.814349084 |
| 2812 | 2583 | TTCATGTGTCCAATATGTGGGGCAGAATTTactcagaaaggtaacctcctccgccacattaaaCTG | 46F_47M_49P_50I_52G_53A_54E_55F_67L | 2.811579609 |
| 2813 | 476 | TTCTGCTGTCCCGTCTGTGGGGCAACTTTTactcagaaaggtaacctcctccgccacattaaaCTG | 46F_47C_49P_50V_52G_53A_54T_55F_67L | 2.808329683 |
| 2814 | 2625 | TTCATGTGTCCAGTTTGTGGGGCAGGCTTTactcagaaaggtaacctcctccgccacattaaaCTG | 46F_47M_49P_50V_52G_53A_54G_55F_67L | 2.806530966 |
| 2815 | 6596 | CTGCACTGCCCTATCTGTGGCTTCACATGTactcagaaaggtaacctcctccgccacattaaaCTG | 46L_47H_49P_50I_52G_53F_54T_55C_67L | 2.803954853 |
| 2816 | 7966 | CTGTACTGCGAGATCTGTGGCTTCCATTGTactcagaaaggtaacctcctccgccacattaaaCTG | 46L_47Y_49E_50I_52G_53F_54H_55C_67L | 2.800208414 |
| 2817 | 4181 | TTCTGGTGTGAAGTCTGTGGGGCATGCTTTactcagaaaggtaacctcctccgccacattaaaCTG | 46F_47W_49E_50V_52G_53A_54C_55F_67L | 2.79841656 |
| 2818 | 6480 | CTGTGTTGCCCCATCTGTGGCTTTGCATGCactcagaaaggtaacctcctccgccacattaaaCTG | 46L_47C_49P_50I_52G_53F_54A_55C_67L | 2.797481955 |
| 2819 | 6481 | CTGTGCTGTCCGATTTGTGGATTCTGTTGTactcagaaaggtaacctcctccgccacattaaaCTG | 46L_47C_49P_50I_52G_53F_54C_55C_67L | 2.794670006 |
| 2820 | 6584 | CTGCACTGCCCAATATGTGGCTTCTTTTGTactcagaaaggtaacctcctccgccacattaaaCTG | 46L_47H_49P_50I_52G_53F_54F_55C_67L | 2.792006888 |
| 2821 | 6555 | TTGTTTTGTCCTATATGCGGGTTTTCATGCactcagaaaggtaacctcctccgccacattaaaCTG | 46L_47F_49P_50I_52G_53F_54S_55C_67L | 2.791145303 |
| 2822 | 4397 | TTCTACTGTGAGATATGTGGGGCAGTATTTactcagaaaggtaacctcctccgccacattaaaCTG | 46F_47Y_49E_50I_52G_53A_54V_55F_67L | 2.786695207 |
| 2823 | 3597 | TTCTCTTGTCCAGTGTGTGGGGCAGTGTTTactcagaaaggtaacctcctccgccacattaaaCTG | 46F_47S_49P_50V_52G_53A_54V_55F_67L | 2.786641061 |
| 2824 | 1792 | TTCATCTGTGAGGTGTGTGGGGCACCATTTactcagaaaggtaacctcctccgccacattaaaCTG | 46F_47I_49E_50V_52G_53A_54P_55F_67L | 2.781290757 |
| 2825 | 3833 | TTCACTTGTCCTGTGTGTGGGGCACAGTTTactcagaaaggtaacctcctccgccacattaaaCTG | 46F_47T_49P_50V_52G_53A_54Q_55F_67L | 2.77919281 |
| 2826 | 3820 | TTCACCTGTCCCGTGTGTGGGGCAGCATTTactcagaaaggtaacctcctccgccacattaaaCTG | 46F_47T_49P_50V_52G_53A_54A_55F_67L | 2.776755164 |
| 2827 | 7973 | CTCTATTGCGAGATCTGTGGATTTCAGTGTactcagaaaggtaacctcctccgccacattaaaCTG | 46L_47Y_49E_50I_52G_53F_54Q_55C_67L | 2.775981374 |
| 2828 | 6484 | CTGTGCTGTCCCATCTGTGGGTTTTTCTGCactcagaaaggtaacctcctccgccacattaaaCTG | 46L_47C_49P_50I_52G_53F_54F_55C_67L | 2.773424656 |
| 2829 | 4141 | TTCTGGTGTGAGATCTGTGGGGCATGCTTTactcagaaaggtaacctcctccgccacattaaaCTG | 46F_47W_49E_50I_52G_53A_54C_55F_67L | 2.771095252 |
| 2830 | 2513 | TTCATGTGTGAGGTTTGTGGGGCACAGTTTactcagaaaggtaacctcctccgccacattaaaCTG | 46F_47M_49E_50V_52G_53A_54Q_55F_67L | 2.770683004 |
| 2831 | 4180 | TTCTGGTGTGAGGTCTGTGGGGCAGCCTTTactcagaaaggtaacctcctccgccacattaaaCTG | 46F_47W_49E_50V_52G_53A_54A_55F_67L | 2.768545284 |
| 2832 | 917 | TTCGAGTGTCCAATCTGTGGGGCAGTCTTTactcagaaaggtaacctcctccgccacattaaaCTG | 46F_47E_49P_50I_52G_53A_54V_55F_67L | 2.766341052 |
| 2833 | 1304 | TTCGGCTGTGAAGTCTGTGGGGCATTCTTTactcagaaaggtaacctcctccgccacattaaaCTG | 46F_47G_49E_50V_52G_53A_54F_55F_67L | 2.765739727 |
| 2834 | 3075 | TTCCAGTGTCCTATCTGTGGGGCAAGCTTTactcagaaaggtaacctcctccgccacattaaaCTG | 46F_47Q_49P_50I_52G_53A_54S_55F_67L | 2.763459429 |
| 2835 | 6541 | CTGTTCTGCCCCATATGTGGGTTCTGTTGTactcagaaaggtaacctcctccgccacattaaaCTG | 46L_47F_49P_50I_52G_53F_54C_55C_67L | 2.763072896 |
| 2836 | 6676 | CTGATGTGTCCCATTTGCGGCTTCACCTGCactcagaaaggtaacctcctccgccacattaaaCTG | 46L_47M_49P_50I_52G_53F_54T_55C_67L | 2.761605439 |
| 2837 | 1752 | TTCATCTGTGAGATTTGTGGGGCACCTTTTactcagaaaggtaacctcctccgccacattaaaCTG | 46F_47I_49E_50I_52G_53A_54P_55F_67L | 2.760826345 |
| 2838 | 6493 | CTGTGTTGTCCGATCTGCGGCTTTCAGTGCactcagaaaggtaacctcctccgccacattaaaCTG | 46L_47C_49P_50I_52G_53F_54Q_55C_67L | 2.759678535 |
| 2839 | 2471 | TTCATGTGTGAGATATGTGGGGCAAACTTTactcagaaaggtaacctcctccgccacattaaaCTG | 46F_47M_49E_50I_52G_53A_54N_55F_67L | 2.756769779 |
| 2840 | 2585 | TTCATGTGTCCAATTTGTGGGGCAGGATTTactcagaaaggtaacctcctccgccacattaaaCTG | 46F_47M_49P_50I_52G_53A_54G_55F_67L | 2.756213106 |
| 2841 | 2635 | TTCATGTGTCCGGTCTGTGGGGCATCATTTactcagaaaggtaacctcctccgccacattaaaCTG | 46F_47M_49P_50V_52G_53A_54S_55F_67L | 2.75374625 |
| 2842 | 3117 | TTCCAATGTCCCGTATGTGGGGCAGTTTTTactcagaaaggtaacctcctccgccacattaaaCTG | 46F_47Q_49P_50V_52G_53A_54V_55F_67L | 2.749755086 |
| 2843 | 2500 | TTCATGTGTGAGGTATGTGGGGCAGCTTTTactcagaaaggtaacctcctccgccacattaaaCTG | 46F_47M_49E_50V_52G_53A_54A_55F_67L | 2.748142569 |
| 2844 | 1381 | TTCGGCTGTCCCATTTGTGGGGCATGTTTTactcagaaaggtaacctcctccgccacattaaaCTG | 46F_47G_49P_50I_52G_53A_54C_55F_67L | 2.744565179 |
| 2845 | 2505 | TTCATGTGTGAGGTGTGTGGGGCAGGATTTactcagaaaggtaacctcctccgccacattaaaCTG | 46F_47M_49E_50V_52G_53A_54G_55F_67L | 2.741885515 |
| 2846 | 313 | TTCTGTTGTGAGATATGTGGGGCACAATTTactcagaaaggtaacctcctccgccacattaaaCTG | 46F_47C_49E_50I_52G_53A_54Q_55F_67L | 2.739980199 |
| 2847 | 6806 | CTGTGGTGTCCTATCTGCGGGTTCCACTGTactcagaaaggtaacctcctccgccacattaaaCTG | 46L_47W_49P_50I_52G_53F_54H_55C_67L | 2.736748324 |
| 2848 | 2465 | TTCATGTGTGAGATCTGTGGGGCAGGTTTTactcagaaaggtaacctcctccgccacattaaaCTG | 46F_47M_49E_50I_52G_53A_54G_55F_67L | 2.732866566 |
| 2849 | 6534 | CTCGAGTGTCCCATCTGCGGGTTTCGCTGCactcagaaaggtaacctcctccgccacattaaaCTG | 46L_47E_49P_50I_52G_53F_54R_55C_67L | 2.731388951 |
| 2850 | 6491 | TTGTGCTGTCCAATCTGCGGGTTCAACTGCactcagaaaggtaacctcctccgccacattaaaCTG | 46L_47C_49P_50I_52G_53F_54N_55C_67L | 2.729981754 |
| 2851 | 3821 | TTCACTTGTCCAGTGTGTGGGGCATGTTTTactcagaaaggtaacctcctccgccacattaaaCTG | 46F_47T_49P_50V_52G_53A_54C_55F_67L | 2.729642906 |
| 2852 | 3581 | TTCTCGTGTCCTGTATGTGGGGCATGCTTTactcagaaaggtaacctcctccgccacattaaaCTG | 46F_47S_49P_50V_52G_53A_54C_55F_67L | 2.72891225 |
| 2853 | 1437 | TTCGGCTGTCCGGTGTGTGGGGCAGTGTTTactcagaaaggtaacctcctccgccacattaaaCTG | 46F_47G_49P_50V_52G_53A_54V_55F_67L | 2.722959423 |
| 2854 | 6800 | CTATGGTGCCCCATCTGCGGCTTTGCCTGCactcagaaaggtaacctcctccgccacattaaaCTG | 46L_47W_49P_50I_52G_53F_54A_55C_67L | 2.716729653 |
| 2855 | 6839 | CTCTATTGTCCAATCTGCGGTTTTTATTGCactcagaaaggtaacctcctccgccacattaaaCTG | 46L_47Y_49P_50I_52G_53F_54Y_55C_67L | 2.715454771 |
| 2856 | 2511 | TTCATGTGTGAGGTTTGTGGGGCAAACTTTactcagaaaggtaacctcctccgccacattaaaCTG | 46F_47M_49E_50V_52G_53A_54N_55F_67L | 2.715281404 |
| 2857 | 3557 | TTCAGCTGTCCCATCTGTGGGGCAGTGTTTactcagaaaggtaacctcctccgccacattaaaCTG | 46F_47S_49P_50I_52G_53A_54V_55F_67L | 2.715066926 |
| 2858 | 2941 | TTCCAATGTGAAATCTGTGGGGCATGTTTTactcagaaaggtaacctcctccgccacattaaaCTG | 46F_47Q_49E_50I_52G_53A_54C_55F_67L | 2.712169019 |
| 2859 | 301 | TTCTGCTGTGAAATCTGTGGGGCATGTTTTactcagaaaggtaacctcctccgccacattaaaCTG | 46F_47C_49E_50I_52G_53A_54C_55F_67L | 2.709021801 |
| 2860 | 2473 | TTCATGTGTGAGATCTGTGGGGCACAGTTTactcagaaaggtaacctcctccgccacattaaaCTG | 46F_47M_49E_50I_52G_53A_54Q_55F_67L | 2.704169912 |
| 2861 | 7979 | CTGTATTGTGAGATTTGCGGTTTCTACTGTactcagaaaggtaacctcctccgccacattaaaCTG | 46L_47Y_49E_50I_52G_53F_54Y_55C_67L | 2.70259398 |
| 2862 | 3077 | TTCCAGTGTCCCATCTGTGGGGCAGTGTTTactcagaaaggtaacctcctccgccacattaaaCTG | 46F_47Q_49P_50I_52G_53A_54V_55F_67L | 2.701410513 |
| 2863 | 951 | TTCGAATGTCCGGTTTGTGGGGCAAATTTTactcagaaaggtaacctcctccgccacattaaaCTG | 46F_47E_49P_50V_52G_53A_54N_55F_67L | 2.700114347 |
| 2864 | 3332 | TTCAGATGTCCACAGTGTGGGGCACCATTTactcagaaaggtaacctcctccgccacattaaaCTG | 46F_47R_49P_50Q_52G_53A_54P_55F_67L | 2.699851476 |
| 2865 | 4153 | TTCTGGTGTGAAATTTGTGGGGCACAGTTTactcagaaaggtaacctcctccgccacattaaaCTG | 46F_47W_49E_50I_52G_53A_54Q_55F_67L | 2.697427235 |
| 2866 | 3540 | TTCAGCTGTCCAATATGTGGGGCAGCTTTTactcagaaaggtaacctcctccgccacattaaaCTG | 46F_47S_49P_50I_52G_53A_54A_55F_67L | 2.695080818 |
| 2867 | 4541 | TTCTACTGTCCGGTTTGTGGGGCATGCTTTactcagaaaggtaacctcctccgccacattaaaCTG | 46F_47Y_49P_50V_52G_53A_54C_55F_67L | 2.689677665 |
| 2868 | 2232 | TTCCTGTGTGAAATCTGTGGGGCACCATTTactcagaaaggtaacctcctccgccacattaaaCTG | 46F_47L_49E_50I_52G_53A_54P_55F_67L | 2.687704723 |
| 2869 | 3973 | TTCGTATGTAATATTTGTGGGGCACAATTTactcagaaaggtaacctcctccgccacattaaaCTG | 46F_47V_49N_50I_52G_53A_54Q_55F_67L | 2.687664174 |
| 2870 | 6673 | CTGATGTGCCCAATCTGTGGATTTCAGTGCactcagaaaggtaacctcctccgccacattaaaCTG | 46L_47M_49P_50I_52G_53F_54Q_55C_67L | 2.684368424 |
| 2871 | 437 | TTCTGCTGTCCTATATGTGGGGCAGTGTTTactcagaaaggtaacctcctccgccacattaaaCTG | 46F_47C_49P_50I_52G_53A_54V_55F_67L | 2.680438493 |
| 2872 | 6665 | CTTATGTGTCCAATTTGTGGTTTCGGATGCactcagaaaggtaacctcctccgccacattaaaCTG | 46L_47M_49P_50I_52G_53F_54G_55C_67L | 2.676409962 |
| 2873 | 357 | TTCTGTTGTGAGGTGTGTGGGGCAGTCTTTactcagaaaggtaacctcctccgccacattaaaCTG | 46F_47C_49E_50V_52G_53A_54V_55F_67L | 2.675692473 |
| 2874 | 427 | TTCTGTTGTCCTATTTGTGGGGCAATTTTTactcagaaaggtaacctcctccgccacattaaaCTG | 46F_47C_49P_50I_52G_53A_54I_55F_67L | 2.673931962 |
| 2875 | 6761 | TTAACTTGCCCCATTTGTGGCTTCTGCTGCactcagaaaggtaacctcctccgccacattaaaCTG | 46L_47T_49P_50I_52G_53F_54C_55C_67L | 2.671218032 |
| 2876 | 4140 | TTCTGGTGTGAAATTTGTGGGGCAGCTTTTactcagaaaggtaacctcctccgccacattaaaCTG | 46F_47W_49E_50I_52G_53A_54A_55F_67L | 2.669618436 |
| 2877 | 3835 | TTCACCTGTCCAGTTTGTGGGGCAAGCTTTactcagaaaggtaacctcctccgccacattaaaCTG | 46F_47T_49P_50V_52G_53A_54S_55F_67L | 2.669126303 |
| 2878 | 4312 | TTCTGGTGTCCTGTGTGTGGGGCACCATTTactcagaaaggtaacctcctccgccacattaaaCTG | 46F_47W_49P_50V_52G_53A_54P_55F_67L | 2.667922369 |
| 2879 | 4195 | TTCTGGTGTGAGGTGTGTGGGGCATCCTTTactcagaaaggtaacctcctccgccacattaaaCTG | 46F_47W_49E_50V_52G_53A_54S_55F_67L | 2.667468359 |
| 2880 | 6580 | CTGCACTGCCCAATCTGTGGATTCGCGTGCactcagaaaggtaacctcctccgccacattaaaCTG | 46L_47H_49P_50I_52G_53F_54A_55C_67L | 2.666715658 |
| 2881 | 6824 | CTTTACTGTCCCATATGCGGGTTTTTCTGTactcagaaaggtaacctcctccgccacattaaaCTG | 46L_47Y_49P_50I_52G_53F_54F_55C_67L | 2.665128981 |
| 2882 | 901 | TTCGAGTGTCCAATCTGTGGGGCATGCTTTactcagaaaggtaacctcctccgccacattaaaCTG | 46F_47E_49P_50I_52G_53A_54C_55F_67L | 2.656223397 |
| 2883 | 2987 | TTCCAGTGTGAGGTCTGTGGGGCAATATTTactcagaaaggtaacctcctccgccacattaaaCTG | 46F_47Q_49E_50V_52G_53A_54I_55F_67L | 2.654347968 |
| 2884 | 7800 | TTGATGTGTGAAATTTGTGGATTCGCATGCactcagaaaggtaacctcctccgccacattaaaCTG | 46L_47M_49E_50I_52G_53F_54A_55C_67L | 2.651355667 |
| 2885 | 4033 | TTCGTGTGTCCGATTTGTGGGGCACAGTTTactcagaaaggtaacctcctccgccacattaaaCTG | 46F_47V_49P_50I_52G_53A_54Q_55F_67L | 2.650749197 |
| 2886 | 4513 | TTCTACTGTCCTATCTGTGGGGCACAGTTTactcagaaaggtaacctcctccgccacattaaaCTG | 46F_47Y_49P_50I_52G_53A_54Q_55F_67L | 2.650610856 |
| 2887 | 6760 | CTAACCTGCCCAATCTGCGGGTTCGCTTGTactcagaaaggtaacctcctccgccacattaaaCTG | 46L_47T_49P_50I_52G_53F_54A_55C_67L | 2.650317654 |
| 2888 | 7960 | CTGTACTGTGAGATTTGCGGATTCGCTTGTactcagaaaggtaacctcctccgccacattaaaCTG | 46L_47Y_49E_50I_52G_53F_54A_55C_67L | 2.649379729 |
| 2889 | 2623 | TTCATGTGTCCAGTTTGTGGGGCAGAATTTactcagaaaggtaacctcctccgccacattaaaCTG | 46F_47M_49P_50V_52G_53A_54E_55F_67L | 2.64889411 |
| 2890 | 7636 | CTTTGCTGTGAGATCTGTGGTTTTACATGTactcagaaaggtaacctcctccgccacattaaaCTG | 46L_47C_49E_50I_52G_53F_54T_55C_67L | 2.647775944 |
| 2891 | 3792 | TTCACATGTCCCATCTGTGGGGCACCGTTTactcagaaaggtaacctcctccgccacattaaaCTG | 46F_47T_49P_50I_52G_53A_54P_55F_67L | 2.647175402 |
| 2892 | 2352 | TTCTTATGTCCCATCTGTGGGGCACCCTTTactcagaaaggtaacctcctccgccacattaaaCTG | 46F_47L_49P_50I_52G_53A_54P_55F_67L | 2.647114353 |
| 2893 | 6776 | CTGACGTGTCCCATTTGTGGGTTTACGTGTactcagaaaggtaacctcctccgccacattaaaCTG | 46L_47T_49P_50I_52G_53F_54T_55C_67L | 2.646152743 |
| 2894 | 6536 | CTGGAATGTCCGATTTGTGGCTTTACCTGCactcagaaaggtaacctcctccgccacattaaaCTG | 46L_47E_49P_50I_52G_53F_54T_55C_67L | 2.641992677 |
| 2895 | 6744 | CTCTCCTGTCCAATATGTGGCTTTTTTTGCactcagaaaggtaacctcctccgccacattaaaCTG | 46L_47S_49P_50I_52G_53F_54F_55C_67L | 2.640239271 |
| 2896 | 4152 | TTCTGGTGTGAAATTTGTGGGGCACCATTTactcagaaaggtaacctcctccgccacattaaaCTG | 46F_47W_49E_50I_52G_53A_54P_55F_67L | 2.635655207 |
| 2897 | 3107 | TTCCAGTGTCCAGTGTGTGGGGCAATTTTTactcagaaaggtaacctcctccgccacattaaaCTG | 46F_47Q_49P_50V_52G_53A_54I_55F_67L | 2.635535277 |
| 2898 | 3541 | TTCTCATGTCCTATATGTGGGGCATGTTTTactcagaaaggtaacctcctccgccacattaaaCTG | 46F_47S_49P_50I_52G_53A_54C_55F_67L | 2.635260867 |
| 2899 | 4432 | TTCTACTGTGAAGTATGTGGGGCACCATTTactcagaaaggtaacctcctccgccacattaaaCTG | 46F_47Y_49E_50V_52G_53A_54P_55F_67L | 2.634685687 |
| 2900 | 4272 | TTCTGGTGTCCCATCTGTGGGGCACCGTTTactcagaaaggtaacctcctccgccacattaaaCTG | 46F_47W_49P_50I_52G_53A_54P_55F_67L | 2.6303289 |
| 2901 | 4313 | TTCTGGTGTCCAGTGTGTGGGGCACAGTTTactcagaaaggtaacctcctccgccacattaaaCTG | 46F_47W_49P_50V_52G_53A_54Q_55F_67L | 2.629865567 |
| 2902 | 4391 | TTCTACTGTGAAATTTGTGGGGCAAATTTTactcagaaaggtaacctcctccgccacattaaaCTG | 46F_47Y_49E_50I_52G_53A_54N_55F_67L | 2.628664905 |
| 2903 | 7875 | CTAAGATGTGAAATCTGCGGCTTTAGTTGCactcagaaaggtaacctcctccgccacattaaaCTG | 46L_47R_49E_50I_52G_53F_54S_55C_67L | 2.622431116 |
| 2904 | 3180 | TTCCGATGTGAGATATGTGGGGCAGCCTTTactcagaaaggtaacctcctccgccacattaaaCTG | 46F_47R_49E_50I_52G_53A_54A_55F_67L | 2.617110988 |
| 2906 | 6826 | CTCTACTGTCCCATCTGTGGCTTCCACTGCactcagaaaggtaacctcctccgccacattaaaCTG | 46L_47Y_49P_50I_52G_53F_54H_55C_67L | 2.609148999 |
| 2905 | 2593 | TTCATGTGTCCTATCTGTGGGGCACAGTTTactcagaaaggtaacctcctccgccacattaaaCTG | 46F_47M_49P_50I_52G_53A_54Q_55F_67L | 2.615303121 |
| 2907 | 6661 | CTCATGTGCCCCATTTGCGGCTTCTGTTGTactcagaaaggtaacctcctccgccacattaaaCTG | 46L_47M_49P_50I_52G_53F_54C_55C_67L | 2.607030746 |
| 2908 | 2400 | TTCATGTGTGATATTTGTGGGGCAGCCTTTactcagaaaggtaacctcctccgccacattaaaCTG | 46F_47M_49D_50I_52G_53A_54A_55F_67L | 2.604836482 |
| 2909 | 4425 | TTCTACTGTGAGGTGTGTGGGGCAGGATTTactcagaaaggtaacctcctccgccacattaaaCTG | 46F_47Y_49E_50V_52G_53A_54G_55F_67L | 2.603979319 |
| 2910 | 7950 | CTCTGGTGTGAGATATGCGGGTTTATGTGCactcagaaaggtaacctcctccgccacattaaaCTG | 46L_47W_49E_50I_52G_53F_54M_55C_67L | 2.603925054 |
| 2911 | 6820 | CTCTACTGTCCGATCTGTGGCTTTGCTTGTactcagaaaggtaacctcctccgccacattaaaCTG | 46L_47Y_49P_50I_52G_53F_54A_55C_67L | 2.603355813 |
| 2912 | 6546 | CTGTTTTGCCCCATCTGCGGCTTTCACTGTactcagaaaggtaacctcctccgccacattaaaCTG | 46L_47F_49P_50I_52G_53F_54H_55C_67L | 2.603337939 |
| 2913 | 6833 | CTCTACTGCCCTATTTGCGGGTTCCAGTGCactcagaaaggtaacctcctccgccacattaaaCTG | 46L_47Y_49P_50I_52G_53F_54Q_55C_67L | 2.592871033 |
| 2914 | 7696 | CTGTTCTGTGAGATTTGTGGCTTCACGTGCactcagaaaggtaacctcctccgccacattaaaCTG | 46L_47F_49E_50I_52G_53F_54T_55C_67L | 2.592812832 |
| 2915 | 7815 | TTGATGTGTGAAATTTGCGGCTTTAGCTGCactcagaaaggtaacctcctccgccacattaaaCTG | 46L_47M_49E_50I_52G_53F_54S_55C_67L | 2.592298084 |
| 2916 | 3549 | TTCTCTTGTCCCATCTGTGGGGCACTCTTTactcagaaaggtaacctcctccgccacattaaaCTG | 46F_47S_49P_50I_52G_53A_54L_55F_67L | 2.591467767 |
| 2917 | 6466 | TTGGCCTGTCCGATATGTGGTTTTCATTGCactcagaaaggtaacctcctccgccacattaaaCTG | 46L_47A_49P_50I_52G_53F_54H_55C_67L | 2.591247553 |
| 2918 | 6497 | CTTTGCTGCCCGATCTGCGGTTTCGTTTGCactcagaaaggtaacctcctccgccacattaaaCTG | 46L_47C_49P_50I_52G_53F_54V_55C_67L | 2.58888798 |
| 2919 | 4396 | TTCTACTGTGAGATCTGTGGGGCAACATTTactcagaaaggtaacctcctccgccacattaaaCTG | 46F_47Y_49E_50I_52G_53A_54T_55F_67L | 2.587652227 |
| 2920 | 4501 | TTCTATTGTCCTATCTGTGGGGCATGCTTTactcagaaaggtaacctcctccgccacattaaaCTG | 46F_47Y_49P_50I_52G_53A_54C_55F_67L | 2.586792361 |
| 2921 | 1693 | TTCATTTGTGATATATGTGGGGCACAGTTTactcagaaaggtaacctcctccgccacattaaaCTG | 46F_47I_49D_50I_52G_53A_54Q_55F_67L | 2.586246446 |
| 2922 | 6740 | CTTAGCTGCCCCATCTGCGGTTTCGCCTGCactcagaaaggtaacctcctccgccacattaaaCTG | 46L_47S_49P_50I_52G_53F_54A_55C_67L | 2.585745182 |
| 2923 | 1912 | TTCATCTGTCCAGTCTGTGGGGCACCCTTTactcagaaaggtaacctcctccgccacattaaaCTG | 46F_47I_49P_50V_52G_53A_54P_55F_67L | 2.584645168 |
| 2924 | 1279 | TTCGGATGTGAGATCTGTGGGGCATATTTTactcagaaaggtaacctcctccgccacattaaaCTG | 46F_47G_49E_50I_52G_53A_54Y_55F_67L | 2.583211228 |
| 2925 | 6478 | CTCGCCTGCCCGATTTGCGGGTTCTGGTGCactcagaaaggtaacctcctccgccacattaaaCTG | 46L_47A_49P_50I_52G_53F_54W_55C_67L | 2.581591668 |
| 2926 | 1264 | TTCGGCTGTGAGATTTGTGGGGCATTCTTTactcagaaaggtaacctcctccgccacattaaaCTG | 46F_47G_49E_50I_52G_53A_54F_55F_67L | 2.579593764 |
| 2927 | 4386 | TTCTACTGTGAGATTTGTGGGGCACACTTTactcagaaaggtaacctcctccgccacattaaaCTG | 46F_47Y_49E_50I_52G_53A_54H_55F_67L | 2.57491496 |
| 2928 | 3116 | TTCCAGTGTCCTGTATGTGGGGCAACTTTTactcagaaaggtaacctcctccgccacattaaaCTG | 46F_47Q_49P_50V_52G_53A_54T_55F_67L | 2.574374287 |
| 2929 | 3460 | TTCAGCTGTGAGGTCTGTGGGGCAGCTTTTactcagaaaggtaacctcctccgccacattaaaCTG | 46F_47S_49E_50V_52G_53A_54A_55F_67L | 2.571965739 |
| 2930 | 6672 | CTGATGTGTCCTATTTGCGGCTTCCCGTGCactcagaaaggtaacctcctccgccacattaaaCTG | 46L_47M_49P_50I_52G_53F_54P_55C_67L | 2.568952268 |
| 2931 | 1435 | TTCGGCTGTCCGGTGTGTGGGGCATCATTTactcagaaaggtaacctcctccgccacattaaaCTG | 46F_47G_49P_50V_52G_53A_54S_55F_67L | 2.568003575 |
| 2932 | 6487 | TTGTGCTGTCCTATATGCGGATTTATTTGTactcagaaaggtaacctcctccgccacattaaaCTG | 46L_47C_49P_50I_52G_53F_54I_55C_67L | 2.565036346 |
| 2933 | 4301 | TTCTGGTGTCCGGTTTGTGGGGCATGCTTTactcagaaaggtaacctcctccgccacattaaaCTG | 46F_47W_49P_50V_52G_53A_54C_55F_67L | 2.563997597 |
| 2934 | 4155 | TTCTGGTGTGAGATTTGTGGGGCATCTTTTactcagaaaggtaacctcctccgccacattaaaCTG | 46F_47W_49E_50I_52G_53A_54S_55F_67L | 2.562652269 |
| 2935 | 4431 | TTCTATTGTGAGGTATGTGGGGCAAACTTTactcagaaaggtaacctcctccgccacattaaaCTG | 46F_47Y_49E_50V_52G_53A_54N_55F_67L | 2.559712751 |
| 2936 | 1033 | TTCTTCTGTGAGATCTGTGGGGCACAGTTTactcagaaaggtaacctcctccgccacattaaaCTG | 46F_47F_49E_50I_52G_53A_54Q_55F_67L | 2.559214366 |
| 2937 | 3060 | TTCCAATGTCCTATATGTGGGGCAGCTTTTactcagaaaggtaacctcctccgccacattaaaCTG | 46F_47Q_49P_50I_52G_53A_54A_55F_67L | 2.558971985 |
| 2938 | 1061 | TTCTTCTGTGAGGTGTGTGGGGCATGCTTTactcagaaaggtaacctcctccgccacattaaaCTG | 46F_47F_49E_50V_52G_53A_54C_55F_67L | 2.557891098 |
| 2939 | 3421 | TTCAGCTGTGAAATCTGTGGGGCATGCTTTactcagaaaggtaacctcctccgccacattaaaCTG | 46F_47S_49E_50I_52G_53A_54C_55F_67L | 2.557690074 |
| 2940 | 6586 | CTTCATTGCCCAATTTGCGGATTCCACTGTactcagaaaggtaacctcctccgccacattaaaCTG | 46L_47H_49P_50I_52G_53F_54H_55C_67L | 2.554831873 |
| 2941 | 356 | TTCTGCTGTGAAGTGTGTGGGGCAACCTTTactcagaaaggtaacctcctccgccacattaaaCTG | 46F_47C_49E_50V_52G_53A_54T_55F_67L | 2.55448039 |
| 2942 | 1152 | TTCTTCTGTCCAATTTGTGGGGCACCATTTactcagaaaggtaacctcctccgccacattaaaCTG | 46F_47F_49P_50I_52G_53A_54P_55F_67L | 2.554134953 |
| 2943 | 1020 | TTCTTCTGTGAGATCTGTGGGGCAGCTTTTactcagaaaggtaacctcctccgccacattaaaCTG | 46F_47F_49E_50I_52G_53A_54A_55F_67L | 2.553491207 |
| 2944 | 4390 | TTCTACTGTGAGATATGTGGGGCAATGTTTactcagaaaggtaacctcctccgccacattaaaCTG | 46F_47Y_49E_50I_52G_53A_54M_55F_67L | 2.551690645 |
| 2945 | 7813 | CTTATGTGCGAGATTTGTGGGTTCCAGTGCactcagaaaggtaacctcctccgccacattaaaCTG | 46L_47M_49E_50I_52G_53F_54Q_55C_67L | 2.551050666 |
| 2946 | 316 | TTCTGCTGTGAGATCTGTGGGGCAACCTTTactcagaaaggtaacctcctccgccacattaaaCTG | 46F_47C_49E_50I_52G_53A_54T_55F_67L | 2.548071898 |
| 2947 | 1387 | TTCGGTTGTCCCATCTGTGGGGCAATTTTTactcagaaaggtaacctcctccgccacattaaaCTG | 46F_47G_49P_50I_52G_53A_54I_55F_67L | 2.546934009 |
| 2948 | 6716 | CTGCAATGCCCCATCTGCGGGTTTACATGTactcagaaaggtaacctcctccgccacattaaaCTG | 46L_47Q_49P_50I_52G_53F_54T_55C_67L | 2.541910735 |
| 2949 | 2520 | TTCATGTGTAACATCTGTGGGGCAGCTTTTactcagaaaggtaacctcctccgccacattaaaCTG | 46F_47M_49N_50I_52G_53A_54A_55F_67L | 2.541109741 |
| 2950 | 3853 | TTCGTTTGTGACATTTGTGGGGCACAATTTactcagaaaggtaacctcctccgccacattaaaCTG | 46F_47V_49D_50I_52G_53A_54Q_55F_67L | 2.539670023 |
| 2951 | 3461 | TTCAGCTGTGAAGTCTGTGGGGCATGCTTTactcagaaaggtaacctcctccgccacattaaaCTG | 46F_47S_49E_50V_52G_53A_54C_55F_67L | 2.539189413 |
| 2952 | 4515 | TTCTACTGTCCGATCTGTGGGGCAAGTTTTactcagaaaggtaacctcctccgccacattaaaCTG | 46F_47Y_49P_50I_52G_53A_54S_55F_67L | 2.538724209 |
| 2953 | 6755 | CTTTCGTGTCCCATTTGCGGTTTCTCATGCactcagaaaggtaacctcctccgccacattaaaCTG | 46L_47S_49P_50I_52G_53F_54S_55C_67L | 2.538069101 |
| 2954 | 6598 | CTGCACTGTCCAATCTGCGGCTTCTGGTGCactcagaaaggtaacctcctccgccacattaaaCTG | 46L_47H_49P_50I_52G_53F_54W_55C_67L | 2.535738839 |
| 2955 | 3955 | TTCGTATGTGAGGTTTGTGGGGCATCCTTTactcagaaaggtaacctcctccgccacattaaaCTG | 46F_47V_49E_50V_52G_53A_54S_55F_67L | 2.535387632 |
| 2956 | 2461 | TTCATGTGTGAGATTTGTGGGGCATGTTTTactcagaaaggtaacctcctccgccacattaaaCTG | 46F_47M_49E_50I_52G_53A_54C_55F_67L | 2.533186829 |
| 2957 | 7953 | CTGTGGTGTGAGATTTGTGGCTTCCAGTGCactcagaaaggtaacctcctccgccacattaaaCTG | 46L_47W_49E_50I_52G_53F_54Q_55C_67L | 2.533161303 |
| 2958 | 7970 | CTCTATTGTGAGATCTGTGGATTTATGTGTactcagaaaggtaacctcctccgccacattaaaCTG | 46L_47Y_49E_50I_52G_53F_54M_55C_67L | 2.532983583 |
| 2959 | 3072 | TTCCAATGTCCTATTTGTGGGGCACCATTTactcagaaaggtaacctcctccgccacattaaaCTG | 46F_47Q_49P_50I_52G_53A_54P_55F_67L | 2.532135299 |
| 2960 | 3061 | TTCCAGTGTCCTATCTGTGGGGCATGTTTTactcagaaaggtaacctcctccgccacattaaaCTG | 46F_47Q_49P_50I_52G_53A_54C_55F_67L | 2.531384643 |
| 2961 | 1757 | TTCATCTGTGAGATTTGTGGGGCAGTGTTTactcagaaaggtaacctcctccgccacattaaaCTG | 46F_47I_49E_50I_52G_53A_54V_55F_67L | 2.529344497 |
| 2962 | 7695 | CTTTTCTGCGAAATTTGTGGCTTCTCCTGCactcagaaaggtaacctcctccgccacattaaaCTG | 46L_47F_49E_50I_52G_53F_54S_55C_67L | 2.526303404 |
| 2963 | 3715 | TTCACATGTGAAGTGTGTGGGGCATCGTTTactcagaaaggtaacctcctccgccacattaaaCTG | 46F_47T_49E_50V_52G_53A_54S_55F_67L | 2.525493765 |
| 2964 | 1781 | TTCATCTGTGAAGTGTGTGGGGCATGTTTTactcagaaaggtaacctcctccgccacattaaaCTG | 46F_47I_49E_50V_52G_53A_54C_55F_67L | 2.5240386 |
| 2965 | 941 | TTCGAGTGTCCTGTTTGTGGGGCATGCTTTactcagaaaggtaacctcctccgccacattaaaCTG | 46F_47E_49P_50V_52G_53A_54C_55F_67L | 2.523993675 |
| 2966 | 6479 | CTTGCATGCCCGATCTGCGGCTTTTACTGTactcagaaaggtaacctcctccgccacattaaaCTG | 46L_47A_49P_50I_52G_53F_54Y_55C_67L | 2.520255649 |
| 2967 | 4273 | TTCTGGTGTCCAATTTGTGGGGCACAGTTTactcagaaaggtaacctcctccgccacattaaaCTG | 46F_47W_49P_50I_52G_53A_54Q_55F_67L | 2.519496334 |
| 2968 | 3100 | TTCCAGTGTCCAGTTTGTGGGGCAGCCTTTactcagaaaggtaacctcctccgccacattaaaCTG | 46F_47Q_49P_50V_52G_53A_54A_55F_67L | 2.518974194 |
| 2969 | 2572 | TTCATGTGTAATGTATGTGGGGCACCGTTTactcagaaaggtaacctcctccgccacattaaaCTG | 46F_47M_49N_50V_52G_53A_54P_55F_67L | 2.517818856 |
| 2970 | 467 | TTCTGCTGTCCCGTTTGTGGGGCAATTTTTactcagaaaggtaacctcctccgccacattaaaCTG | 46F_47C_49P_50V_52G_53A_54I_55F_67L | 2.517587678 |
| 2971 | 1872 | TTCATCTGTCCTATTTGTGGGGCACCTTTTactcagaaaggtaacctcctccgccacattaaaCTG | 46F_47I_49P_50I_52G_53A_54P_55F_67L | 2.515857496 |
| 2972 | 7935 | TTAGTGTGCGAAATCTGCGGCTTCTCCTGCactcagaaaggtaacctcctccgccacattaaaCTG | 46L_47V_49E_50I_52G_53F_54S_55C_67L | 2.515093282 |
| 2973 | 6666 | CTGATGTGTCCCATCTGCGGATTTCACTGCactcagaaaggtaacctcctccgccacattaaaCTG | 46L_47M_49P_50I_52G_53F_54H_55C_67L | 2.51368859 |
| 2974 | 6476 | TTAGCCTGTCCCATTTGCGGGTTTACATGCactcagaaaggtaacctcctccgccacattaaaCTG | 46L_47A_49P_50I_52G_53F_54T_55C_67L | 2.513019342 |
| 2975 | 4197 | TTCTGGTGTGAGGTGTGTGGGGCAGTCTTTactcagaaaggtaacctcctccgccacattaaaCTG | 46F_47W_49E_50V_52G_53A_54V_55F_67L | 2.512663654 |
| 2976 | 3070 | TTCCAATGTCCAATCTGTGGGGCAATGTTTactcagaaaggtaacctcctccgccacattaaaCTG | 46F_47Q_49P_50I_52G_53A_54M_55F_67L | 2.509501967 |
| 2977 | 3701 | TTCACTTGTGAGGTCTGTGGGGCATGTTTTactcagaaaggtaacctcctccgccacattaaaCTG | 46F_47T_49E_50V_52G_53A_54C_55F_67L | 2.50853845 |
| 2978 | 3113 | TTCCAGTGTCCCGTGTGTGGGGCACAGTTTactcagaaaggtaacctcctccgccacattaaaCTG | 46F_47Q_49P_50V_52G_53A_54Q_55F_67L | 2.508246727 |
| 2979 | 3063 | TTCCAATGTCCAATTTGTGGGGCAGAGTTTactcagaaaggtaacctcctccgccacattaaaCTG | 46F_47Q_49P_50I_52G_53A_54E_55F_67L | 2.5065491 |
| 2980 | 4157 | TTCTGGTGTGAAATTTGTGGGGCAGTATTTactcagaaaggtaacctcctccgccacattaaaCTG | 46F_47W_49E_50I_52G_53A_54V_55F_67L | 2.504575914 |
| 2981 | 3837 | TTCACGTGTCCAGTGTGTGGGGCAGTCTTTactcagaaaggtaacctcctccgccacattaaaCTG | 46F_47T_49P_50V_52G_53A_54V_55F_67L | 2.503738426 |
| 2982 | 7936 | CTGGTGTGTGAAATCTGCGGGTTCACATGTactcagaaaggtaacctcctccgccacattaaaCTG | 46L_47V_49E_50I_52G_53F_54T_55C_67L | 2.503150926 |
| 2983 | 2477 | TTCATGTGTGAGATATGTGGGGCAGTTTTTactcagaaaggtaacctcctccgccacattaaaCTG | 46F_47M_49E_50I_52G_53A_54V_55F_67L | 2.500832147 |
| 2984 | 307 | TTCTGCTGTGAAATCTGTGGGGCAATATTTactcagaaaggtaacctcctccgccacattaaaCTG | 46F_47C_49E_50I_52G_53A_54I_55F_67L | 2.499075416 |
| 2985 | 3675 | TTCACATGTGAAATATGTGGGGCAAGTTTTactcagaaaggtaacctcctccgccacattaaaCTG | 46F_47T_49E_50I_52G_53A_54S_55F_67L | 2.496047656 |
| 2986 | 1427 | TTCGGATGTCCCGTCTGTGGGGCAATATTTactcagaaaggtaacctcctccgccacattaaaCTG | 46F_47G_49P_50V_52G_53A_54I_55F_67L | 2.495943151 |
| 2987 | 1672 | TTCCATTGTCCAGTGTGTGGGGCACCCTTTactcagaaaggtaacctcctccgccacattaaaCTG | 46F_47H_49P_50V_52G_53A_54P_55F_67L | 2.495283347 |
| 2988 | 475 | TTCTGTTGTCCAGTCTGTGGGGCATCCTTTactcagaaaggtaacctcctccgccacattaaaCTG | 46F_47C_49P_50V_52G_53A_54S_55F_67L | 2.49413606 |
| 2989 | 3437 | TTCTCTTGTGAAATTTGTGGGGCAGTCTTTactcagaaaggtaacctcctccgccacattaaaCTG | 46F_47S_49E_50I_52G_53A_54V_55F_67L | 2.493648415 |
| 2990 | 2581 | TTCATGTGTCCGATATGTGGGGCATGTTTTactcagaaaggtaacctcctccgccacattaaaCTG | 46F_47M_49P_50I_52G_53A_54C_55F_67L | 2.493278422 |
| 2991 | 6556 | CTGTTTTGTCCCATCTGCGGGTTCACATGTactcagaaaggtaacctcctccgccacattaaaCTG | 46L_47F_49P_50I_52G_53F_54T_55C_67L | 2.493210153 |
| 2992 | 1633 | TTCCACTGTCCGATCTGTGGGGCACAGTTTactcagaaaggtaacctcctccgccacattaaaCTG | 46F_47H_49P_50I_52G_53A_54Q_55F_67L | 2.492791165 |
| 2993 | 1395 | TTCGGCTGTCCCATCTGTGGGGCATCATTTactcagaaaggtaacctcctccgccacattaaaCTG | 46F_47G_49P_50I_52G_53A_54S_55F_67L | 2.492491621 |
| 2994 | 1153 | TTCTTTTGTCCTATTTGTGGGGCACAGTTTactcagaaaggtaacctcctccgccacattaaaCTG | 46F_47F_49P_50I_52G_53A_54Q_55F_67L | 2.491482283 |
| 2995 | 3912 | TTCGTGTGTGAAATCTGTGGGGCACCCTTTactcagaaaggtaacctcctccgccacattaaaCTG | 46F_47V_49E_50I_52G_53A_54P_55F_67L | 2.489619044 |
| 2996 | 3661 | TTCACATGTGAAATTTGTGGGGCATGCTTTactcagaaaggtaacctcctccgccacattaaaCTG | 46F_47T_49E_50I_52G_53A_54C_55F_67L | 2.487404494 |
| 2997 | 4559 | TTCTACTGTCCTGTTTGTGGGGCATACTTTactcagaaaggtaacctcctccgccacattaaaCTG | 46F_47Y_49P_50V_52G_53A_54Y_55F_67L | 2.484380202 |
| 2998 | 420 | TTCTGCTGTCCTATCTGTGGGGCAGCCTTTactcagaaaggtaacctcctccgccacattaaaCTG | 46F_47C_49P_50I_52G_53A_54A_55F_67L | 2.48350377 |
| 2999 | 460 | TTCTGTTGTCCTGTGTGTGGGGCAGCCTTTactcagaaaggtaacctcctccgccacattaaaCTG | 46F_47C_49P_50V_52G_53A_54A_55F_67L | 2.4834915 |
| 3000 | 1075 | TTCTTTTGTGAAGTCTGTGGGGCAAGCTTTactcagaaaggtaacctcctccgccacattaaaCTG | 46F_47F_49E_50V_52G_53A_54S_55F_67L | 2.483339126 |
| 3001 | 6756 | CTCAGCTGTCCTATTTGTGGATTTACCTGTactcagaaaggtaacctcctccgccacattaaaCTG | 46L_47S_49P_50I_52G_53F_54T_55C_67L | 2.482181757 |
| 3002 | 3300 | TTCCGCTGTCCAATCTGTGGGGCAGCCTTTactcagaaaggtaacctcctccgccacattaaaCTG | 46F_47R_49P_50I_52G_53A_54A_55F_67L | 2.481052868 |
| 3003 | 6825 | TTGTACTGTCCCATCTGTGGTTTCGGTTGCactcagaaaggtaacctcctccgccacattaaaCTG | 46L_47Y_49P_50I_52G_53F_54G_55C_67L | 2.480729656 |
| 3004 | 7954 | CTTTGGTGTGAAATCTGCGGATTCAGATGTactcagaaaggtaacctcctccgccacattaaaCTG | 46L_47W_49E_50I_52G_53F_54R_55C_67L | 2.479274838 |
| 3005 | 3780 | TTCACGTGTCCAATCTGTGGGGCAGCTTTTactcagaaaggtaacctcctcogccacattaaaCTG | 46F_47T_49P_50I_52G_53A_54A_55F_67L | 2.477394408 |
| 3006 | 6475 | TTGGCGTGTCCAATTTGCGGATTCTCATGTactcagaaaggtaacctcctccgccacattaaaCTG | 46L_47A_49P_50I_52G_53F_54S_55C_67L | 2.477315255 |
| 3007 | 3062 | TTCCAGTGTCCCATCTGTGGGGCAGACTTTactcagaaaggtaacctcctccgccacattaaaCTG | 46F_47Q_49P_50I_52G_53A_54D_55F_67L | 2.475085294 |
| 3008 | 6498 | TTGTGCTGCCCAATCTGCGGCTTCTGGTGTactcagaaaggtaacctcctccgccacattaaaCTG | 46L_47C_49P_50I_52G_53F_54W_55C_67L | 2.474894822 |
| 3009 | 3672 | TTCACGTGTGAGATCTGTGGGGCACCCTTTactcagaaaggtaacctcctccgccacattaaaCTG | 46F_47T_49E_50I_52G_53A_54P_55F_67L | 2.474058076 |
| 3010 | 6664 | TTAATGTGCCCTATTTGCGGCTTTTTTTGCactcagaaaggtaacctcctccgccacattaaaCTG | 46L_47M_49P_50I_52G_53F_54F_55C_67L | 2.472233212 |
| 3011 | 6766 | CTCACATGCCCAATCTGTGGGTTCCACTGTactcagaaaggtaacctcctccgccacattaaaCTG | 46L_47T_49P_50I_52G_53F_54H_55C_67L | 2.472004298 |
| 3012 | 6700 | CTCCAGTGCCCGATTTGTGGGTTTGCTTGTactcagaaaggtaacctcctccgccacattaaaCTG | 46L_47Q_49P_50I_52G_53F_54A_55C_67L | 2.470662226 |
| 3013 | 6539 | CTGGAGTGCCCAATCTGTGGATTCTACTGCactcagaaaggtaacctcctccgccacattaaaCTG | 46L_47E_49P_50I_52G_53F_54Y_55C_67L | 2.470119293 |
| 3014 | 317 | TTCTGTTGTGAGATTTGTGGGGCAGTGTTTactcagaaaggtaacctcctccgccacattaaaCTG | 46F_47C_49E_50I_52G_53A_54V_55F_67L | 2.47007553 |
| 3015 | 4436 | TTCTACTGTGAGGTTTGTGGGGCAACGTTTactcagaaaggtaacctcctccgccacattaaaCTG | 46F_47Y_49E_50V_52G_53A_54T_55F_67L | 2.466835445 |
| 3016 | 6581 | CTGCACTGCCCAATTTGCGGGTTCTGCTGTactcagaaaggtaacctcctccgccacattaaaCTG | 46L_47H_49P_50I_52G_53F_54C_55C_67L | 2.462237201 |
| 3017 | 3185 | TTCCGTTGTGAGATCTGTGGGGCAGGTTTTactcagaaaggtaacctcctccgccacattaaaCTG | 46F_47R_49E_50I_52G_53A_54G_55F_67L | 2.461267664 |
| 3018 | 6579 | CTCGGGTGCCCAATCTGCGGCTTCTACTGCactcagaaaggtaacctcctccgccacattaaaCTG | 46L_47G_49P_50I_52G_53F_54Y_55C_67L | 2.460052562 |
| 3019 | 957 | TTCGAGTGTCCAGTGTGTGGGGCAGTGTTTactcagaaaggtaacctcctccgccacattaaaCTG | 46F_47E_49P_50V_52G_53A_54V_55F_67L | 2.45812958 |
| 3020 | 2950 | TTCCAGTGTGAGATCTGTGGGGCAATGTTTactcagaaaggtaacctcctccgccacattaaaCTG | 46F_47Q_49E_50I_52G_53A_54M_55F_67L | 2.453044593 |
| 3021 | 2501 | TTCATGTGTGAGGTGTGTGGGGCATGTTTTactcagaaaggtaacctcctccgccacattaaaCTG | 46F_47M_49E_50V_52G_53A_54C_55F_67L | 2.450469953 |
| 3022 | 361 | TTCTGTTGTAACATCTGTGGGGCATGTTTTactcagaaaggtaacctcctccgccacattaaaCTG | 46F_47C_49N_50I_52G_53A_54C_55F_67L | 2.450360342 |
| 3023 | 4426 | TTCTACTGTGAGGTGTGTGGGGCACATTTTactcagaaaggtaacctcctccgccacattaaaCTG | 46F_47Y_49E_50V_52G_53A_54H_55F_67L | 2.449972019 |
| 3024 | 2452 | TTCATGTGTGACGTATGTGGGGCACCTTTTactcagaaaggtaacctcctccgccacattaaaCTG | 46F_47M_49D_50V_52G_53A_54P_55F_67L | 2.449568179 |
| 3025 | 1741 | TTCATCTGTGAGATTTGTGGGGCATGCTTTactcagaaaggtaacctcctccgccacattaaaCTG | 46F_47I_49E_50I_52G_53A_54C_55F_67L | 2.449346068 |
| 3026 | 353 | TTCTGCTGTGAGGTGTGTGGGGCACAGTTTactcagaaaggtaacctcctccgccacattaaaCTG | 46F_47C_49E_50V_52G_53A_54Q_55F_67L | 2.446482883 |
| 3027 | 2990 | TTCCAGTGTGAGGTGTGTGGGGCAATGTTTactcagaaaggtaacctcctccgccacattaaaCTG | 46F_47Q_49E_50V_52G_53A_54M_55F_67L | 2.442838925 |
| 3028 | 4300 | TTCTGGTGTCCAGTGTGTGGGGCAGCATTTactcagaaaggtaacctcctccgccacattaaaCTG | 46F_47W_49P_50V_52G_53A_54A_55F_67L | 2.442278776 |
| 3029 | 3825 | TTCACCTGTCCGGTTTGTGGGGCAGGTTTTactcagaaaggtaacctcctccgccacattaaaCTG | 46F_47T_49P_50V_52G_53A_54G_55F_67L | 2.442221396 |
| 3030 | 1740 | TTCATTTGTGAAATCTGTGGGGCAGCATTTactcagaaaggtaacctcctccgccacattaaaCTG | 46F_47I_49E_50I_52G_53A_54A_55F_67L | 2.442165204 |
| 3031 | 6544 | CTGTTCTGCCCCATCTGCGGATTCTTTTGTactcagaaaggtaacctcctccgccacattaaaCTG | 46L_47F_49P_50I_52G_53F_54F_55C_67L | 2.441566382 |
| 3032 | 1795 | TTCATATGTGAGGTCTGTGGGGCATCGTTTactcagaaaggtaacctcctccgccacattaaaCTG | 46F_47I_49E_50V_52G_53A_54S_55F_67L | 2.441346886 |
| 3033 | 3340 | TTCAGGTGTCCAGTATGTGGGGCAGCTTTTactcagaaaggtaacctcctccgccacattaaaCTG | 46F_47R_49P_50V_52G_53A_54A_55F_67L | 2.440898463 |
| 3034 | 6735 | CTGCGCTGTCCCATATGTGGTTTCAGCTGTactcagaaaggtaacctcctccgccacattaaaCTG | 46L_47R_49P_50I_52G_53F_54S_55C_67L | 2.439988226 |
| 3035 | 3972 | TTCGTCTGTAACATTTGTGGGGCACCCTTTactcagaaaggtaacctcctccgccacattaaaCTG | 46F_47V_49N_50I_52G_53A_54P_55F_67L | 2.439121381 |
| 3036 | 6540 | CTGTTCTGCCCGATCTGCGGTTTCGCCTGCactcagaaaggtaacctcctccgccacattaaaCTG | 46L_47F_49P_50I_52G_53F_54A_55C_67L | 2.437112204 |
| 3037 | 435 | TTCTGTTGTCCAATTTGTGGGGCATCCTTTactcagaaaggtaacctcctccgccacattaaaCTG | 46F_47C_49P_50I_52G_53A_54S_55F_67L | 2.435524506 |
| 3038 | 7977 | CTGTACTGCGAAATTTGCGGCTTTGTGTGTactcagaaaggtaacctcctccgccacattaaaCTG | 46L_47Y_49E_50I_52G_53F_54V_55C_67L | 2.434584387 |
| 3039 | 6741 | CTGAGCTGTCCCATCTGTGGCTTCTGCTGCactcagaaaggtaacctcctccgccacattaaaCTG | 46L_47S_49P_50I_52G_53F_54C_55C_67L | 2.434040284 |
| 3040 | 6830 | TTATATTGTCCTATCTGTGGATTCATGTGCactcagaaaggtaacctcctccgccacattaaaCTG | 46L_47Y_49P_50I_52G_53F_54M_55C_67L | 2.43291547 |
| 3042 | 3797 | TTCACCTGTCCTATTTGTGGGGCAGTGTTTactcagaaaggtaacctcctccgccacattaaaCTG | 46F_47T_49P_50I_52G_53A_54V_55F_67L | 2.431785701 |
| 3041 | 4452 | TTCTACTGTAATATTTGTGGGGCACCATTTactcagaaaggtaacctcctccgccacattaaaCTG | 46F_47Y_49N_50I_52G_53A_54P_55F_67L | 2.432817843 |
| 3043 | 4430 | TTCTATTGTGAAGTTTGTGGGGCAATGTTTactcagaaaggtaacctcctccgccacattaaaCTG | 46F_47Y_49E_50V_52G_53A_54M_55F_67L | 2.430918064 |
| 3044 | 4427 | TTCTATTGTGAAGTTTGTGGGGCAATCTTTactcagaaaggtaacctcctccgccacattaaaCTG | 46F_47Y_49E_50V_52G_53A_54I_55F_67L | 2.429161133 |
| 3045 | 7964 | CTGTACTGCGAAATCTGCGGGTTTTTTTGCactcagaaaggtaacctcctccgccacattaaaCTG | 46L_47Y_49E_50I_52G_53F_54F_55C_67L | 2.428120001 |
| 3046 | 6663 | CTGATGTGCCCAATTTGCGGTTTTGAGTGTactcagaaaggtaacctcctccgccacattaaaCTG | 46L_47M_49P_50I_52G_53F_54E_55C_67L | 2.427156976 |
| 3047 | 4503 | TTCTACTGTCCGATTTGTGGGGCAGAATTTactcagaaaggtaacctcctccgccacattaaaCTG | 46F_47Y_49P_50I_52G_53A_54E_55F_67L | 2.427107039 |
| 3048 | 1193 | TTCTTCTGTCCAGTGTGTGGGGCACAGTTTactcagaaaggtaacctcctcogccacattaaaCTG | 46F_47F_49P_50V_52G_53A_54Q_55F_67L | 2.427088728 |
| 3049 | 1625 | TTCCACTGTCCTATTTGTGGGGCAGGATTTactcagaaaggtaacctcctccgccacattaaaCTG | 46F_47H_49P_50I_52G_53A_54G_55F_67L | 2.426675421 |
| 3050 | 7736 | TTGCATTGCGAAATATGTGGCTTCACTTGTactcagaaaggtaacctcctccgccacattaaaCTG | 46L_47H_49E_50I_52G_53F_54T_55C_67L | 2.425460875 |
| 3051 | 6778 | CTGACATGTCCAATATGCGGTTTCTGGTGCactcagaaaggtaacctcctccgccacattaaaCTG | 46L_47T_49P_50I_52G_53F_54W_55C_67L | 2.424904029 |
| 3052 | 4517 | TTCTACTGTCCTATTTGTGGGGCAGTGTTTactcagaaaggtaacctcctccgccacattaaaCTG | 46F_47Y_49P_50I_52G_53A_54V_55F_67L | 2.420084589 |
| 3053 | 3595 | TTCAGCTGTCCCGTTTGTGGGGCATCCTTTactcagaaaggtaacctcctccgccacattaaaCTG | 46F_47S_49P_50V_52G_53A_54S_55F_67L | 2.419013805 |
| 3054 | 4516 | TTCTATTGTCCAATCTGTGGGGCAACATTTactcagaaaggtaacctcctccgccacattaaaCTG | 46F_47Y_49P_50I_52G_53A_54T_55F_67L | 2.419009589 |
| 3055 | 4315 | TTCTGGTGTCCCGTATGTGGGGCATCTTTTactcagaaaggtaacctcctccgccacattaaaCTG | 46F_47W_49P_50V_52G_53A_54S_55F_67L | 2.41677948 |
| 3056 | 1073 | TTCTTCTGTGAGGTCTGTGGGGCACAGTTTactcagaaaggtaacctcctccgccacattaaaCTG | 46F_47F_49E_50V_52G_53A_54Q_55F_67L | 2.416321978 |
| 3057 | 6535 | CTAGAATGCCCCATCTGTGGTTTTTCGTGTactcagaaaggtaacctcctccgccacattaaaCTG | 46L_47E_49P_50I_52G_53F_54S_55C_67L | 2.415611986 |
| 3058 | 3705 | TTCACTTGTGAGGTGTGTGGGGCAGGTTTTactcagaaaggtaacctcctccgccacattaaaCTG | 46F_47T_49E_50V_52G_53A_54G_55F_67L | 2.41534566 |
| 3059 | 3832 | TTCACTTGTCCCGTCTGTGGGGCACCGTTTactcagaaaggtaacctcctccgccacattaaaCTG | 46F_47T_49P_50V_52G_53A_54P_55F_67L | 2.414748853 |
| 3060 | 1180 | TTCTTCTGTCCCGTGTGTGGGGCAGCATTTactcagaaaggtaacctcctccgccacattaaaCTG | 46F_47F_49P_50V_52G_53A_54A_55F_67L | 2.414632092 |
| 3061 | 3429 | TTCTCCTGTGAGATTTGTGGGGCACTTTTTactcagaaaggtaacctcctccgccacattaaaCTG | 46F_47S_49E_50I_52G_53A_54L_55F_67L | 2.413992681 |
| 3062 | 6715 | TTACAGTGTCCAATCTGCGGATTTAGCTGTactcagaaaggtaacctcctccgccacattaaaCTG | 46L_47Q_49P_50I_52G_53F_54S_55C_67L | 2.411108438 |
| 3063 | 3073 | TTCCAGTGTCCAATCTGTGGGGCACAGTTTactcagaaaggtaacctcctccgccacattaaaCTG | 46F_47Q_49P_50I_52G_53A_54Q_55F_67L | 2.409602958 |
| 3064 | 2953 | TTCCAGTGTGAGATTTGTGGGGCACAGTTTactcagaaaggtaacctcctccgccacattaaaCTG | 46F_47Q_49E_50I_52G_53A_54Q_55F_67L | 2.405803625 |
| 3065 | 4147 | TTCTGGTGTGAGATTTGTGGGGCAATATTTactcagaaaggtaacctcctccgccacattaaaCTG | 46F_47W_49E_50I_52G_53A_54I_55F_67L | 2.405514744 |
| 3066 | 2956 | TTCCAATGTGAGATTTGTGGGGCAACATTTactcagaaaggtaacctcctccgccacattaaaCTG | 46F_47Q_49E_50I_52G_53A_54T_55F_67L | 2.404746358 |
| 3067 | 4279 | TTCTGGTGTCCTATTTGTGGGGCATATTTTactcagaaaggtaacctcctccgccacattaaaCTG | 46F_47W_49P_50I_52G_53A_54Y_55F_67L | 2.404267782 |
| 3068 | 4387 | TTCTACTGTGAAATTTGTGGGGCAATCTTTactcagaaaggtaacctcctccgccacattaaaCTG | 46F_47Y_49E_50I_52G_53A_54I_55F_67L | 2.402307696 |
| 3069 | 4320 | TTCTACTGTGACATATGTGGGGCAGCCTTTactcagaaaggtaacctcctccgccacattaaaCTG | 46F_47Y_49D_50I_52G_53A_54A_55F_67L | 2.401918498 |
| 3070 | 3941 | TTCGTGTGTGAGGTGTGTGGGGCATGCTTTactcagaaaggtaacctcctccgccacattaaaCTG | 46F_47V_49E_50V_52G_53A_54C_55F_67L | 2.399614669 |
| 3071 | 4384 | TTCTATTGTGAGATCTGTGGGGCATTTTTTactcagaaaggtaacctcctccgccacattaaaCTG | 46F_47Y_49E_50I_52G_53A_54F_55F_67L | 2.398674627 |
| 3072 | 4261 | TTCTGGTGTCCAATCTGTGGGGCATGCTTTactcagaaaggtaacctcctccgccacattaaaCTG | 46F_47W_49P_50I_52G_53A_54C_55F_67L | 2.396914114 |
| 3073 | 900 | TTCGAGTGTCCAATCTGTGGGGCAGCGTTTactcagaaaggtaacctcctccgccacattaaaCTG | 46F_47E_49P_50I_52G_53A_54A_55F_67L | 2.394543695 |
| 3074 | 3676 | TTCACTTGTGAAATTTGTGGGGCAACCTTTactcagaaaggtaacctcctccgccacattaaaCTG | 46F_47T_49E_50I_52G_53A_54T_55F_67L | 2.392712407 |
| 3075 | 2997 | TTCCAGTGTGAGGTATGTGGGGCAGTGTTTactcagaaaggtaacctcctccgccacattaaaCTG | 46F_47Q_49E_50V_52G_53A_54V_55F_67L | 2.391803486 |
| 3076 | 7755 | CTTATCTGTGAGATATGCGGATTTTCCTGCactcagaaaggtaacctcctccgccacattaaaCTG | 46L_47I_49E_50I_52G_53F_54S_55C_67L | 2.391633038 |
| 3077 | 1755 | TTCATATGTGAGATTTGTGGGGCATCTTTTactcagaaaggtaacctcctccgccacattaaaCTG | 46F_47I_49E_50I_52G_53A_54S_55F_67L | 2.39051538 |
| 3078 | 3101 | TTCCAATGTCCTGTCTGTGGGGCATGCTTTactcagaaaggtaacctcctccgccacattaaaCTG | 46F_47Q_49P_50V_52G_53A_54C_55F_67L | 2.389574207 |
| 3079 | 4519 | TTCTACTGTCCAATATGTGGGGCATACTTTactcagaaaggtaacctcctccgccacattaaaCTG | 46F_47Y_49P_50I_52G_53A_54Y_55F_67L | 2.388670846 |
| 3080 | 6720 | CTCAGATGTCCTATATGTGGTTTCGCATGCactcagaaaggtaacctcctccgccacattaaaCTG | 46L_47R_49P_50I_52G_53F_54A_55C_67L | 2.388456498 |
| 3081 | 7915 | TTGACTTGCGAGATATGTGGATTCTCCTGCactcagaaaggtaacctcctccgccacattaaaCTG | 46L_47T_49E_50I_52G_53F_54S_55C_67L | 2.388357082 |
| 3082 | 6746 | CTATCTTGCCCTATATGCGGCTTTCACTGCactcagaaaggtaacctcctccgccacattaaaCTG | 46L_47S_49P_50I_52G_53F_54H_55C_67L | 2.388082611 |
| 3083 | 4385 | TTCTATTGTGAGATATGTGGGGCAGGGTTTactcagaaaggtaacctcctccgccacattaaaCTG | 46F_47Y_49E_50I_52G_53A_54G_55F_67L | 2.386567483 |
| 3084 | 6817 | CTCTGGTGCCCCATCTGCGGATTCGTGTGCactcagaaaggtaacctcctccgccacattaaaCTG | 46L_47W_49P_50I_52G_53F_54V_55C_67L | 2.385036544 |
| 3085 | 4511 | TTCTATTGTCCCATTTGTGGGGCAAACTTTactcagaaaggtaacctcctccgccacattaaaCTG | 46F_47Y_49P_50I_52G_53A_54N_55F_67L | 2.38304467 |
| 3086 | 3115 | TTCCAATGTCCTGTGTGTGGGGCATCATTTactcagaaaggtaacctcctcogccacattaaaCTG | 46F_47Q_49P_50V_52G_53A_54S_55F_67L | 2.382850735 |
| 3087 | 4389 | TTCTACTGTGAAATCTGTGGGGCACTGTTTactcagaaaggtaacctcctccgccacattaaaCTG | 46F_47Y_49E_50I_52G_53A_54L_55F_67L | 2.382676439 |
| 3088 | 4500 | TTCTACTGTCCAATCTGTGGGGCAGCATTTactcagaaaggtaacctcctccgccacattaaaCTG | 46F_47Y_49P_50I_52G_53A_54A_55F_67L | 2.3825962 |
| 3089 | 4553 | TTCTACTGTCCGGTGTGTGGGGCACAATTTactcagaaaggtaacctcctccgccacattaaaCTG | 46F_47Y_49P_50V_52G_53A_54Q_55F_67L | 2.38109103 |
| 3090 | 7753 | CTAATCTGTGAGATATGCGGCTTTCAGTGCactcagaaaggtaacctcctccgccacattaaaCTG | 46L_47I_49E_50I_52G_53F_54Q_55C_67L | 2.38099613 |
| 3091 | 915 | TTCGAGTGTCCAATCTGTGGGGCATCTTTTactcagaaaggtaacctcctccgccacattaaaCTG | 46F_47E_49P_50I_52G_53A_54S_55F_67L | 2.377349295 |
| 3092 | 1512 | TTCCATTGTGAGATCTGTGGGGCACCATTTactcagaaaggtaacctcctccgccacattaaaCTG | 46F_47H_49E_50I_52G_53A_54P_55F_67L | 2.376661827 |
| 3093 | 6795 | CTGGTGTGCCCCATCTGCGGCTTCTCTTGTactcagaaaggtaacctcctccgccacattaaaCTG | 46L_47V_49P_50I_52G_53F_54S_55C_67L | 2.376585167 |
| 3094 | 4060 | TTCGTGTGTCCTGTCTGTGGGGCAGCGTTTactcagaaaggtaacctcctccgccacattaaaCTG | 46F_47V_49P_50V_52G_53A_54A_55F_67L | 2.374555549 |
| 3095 | 3102 | TTCCAGTGTCCAGTCTGTGGGGCAGACTTTactcagaaaggtaacctcctccgccacattaaaCTG | 46F_47Q_49P_50V_52G_53A_54D_55F_67L | 2.373599195 |
| 3096 | 1910 | TTCATATGTCCAGTGTGTGGGGCAATGTTTactcagaaaggtaacctcctccgccacattaaaCTG | 46F_47I_49P_50V_52G_53A_54M_55F_67L | 2.371012205 |
| 3097 | 7944 | CTCTGGTGCGAAATATGTGGTTTTTTTTGTactcagaaaggtaacctcctccgccacattaaaCTG | 46L_47W_49E_50I_52G_53F_54F_55C_67L | 2.365701386 |
| 3098 | 347 | TTCTGTTGTGAAGTGTGTGGGGCAATCTTTactcagaaaggtaacctcctccgccacattaaaCTG | 46F_47C_49E_50V_52G_53A_54I_55F_67L | 2.364381529 |
| 3099 | 1324 | TTCGGGTGTAATATATGTGGGGCATTCTTTactcagaaaggtaacctcctccgccacattaaaCTG | 46F_47G_49N_50I_52G_53A_54F_55F_67L | 2.36402202 |
| 3100 | 436 | TTCTGCTGTCCTATCTGTGGGGCAACCTTTactcagaaaggtaacctcctccgccacattaaaCTG | 46F_47C_49P_50I_52G_53A_54T_55F_67L | 2.363418817 |
| 3101 | 6811 | CTGTGGTGTCCCATTTGCGGGTTCAATTGTactcagaaaggtaacctcctccgccacattaaaCTG | 46L_47W_49P_50I_52G_53F_54N_55C_67L | 2.363282755 |
| 3102 | 4540 | TTCTACTGTCCAGTCTGTGGGGCAGCTTTTactcagaaaggtaacctcctccgccacattaaaCTG | 46F_47Y_49P_50V_52G_53A_54A_55F_67L | 2.362887674 |
| 3103 | 2600 | TTCATGTGTCCGCAGTGTGGGGCAGCGTTTactcagaaaggtaacctcctccgccacattaaaCTG | 46F_47M_49P_50Q_52G_53A_54A_55F_67L | 2.357660668 |
| 3104 | 3917 | TTCGTATGTGAGATCTGTGGGGCAGTGTTTactcagaaaggtaacctcctccgccacattaaaCTG | 46F_47V_49E_50I_52G_53A_54V_55F_67L | 2.357622341 |
| 3105 | 6593 | TTGCACTGCCCTATCTGTGGATTTCAATGCactcagaaaggtaacctcctccgccacattaaaCTG | 46L_47H_49P_50I_52G_53F_54Q_55C_67L | 2.355139708 |
| 3106 | 6585 | CTACATTGTCCTATCTGCGGCTTTGGGTGTactcagaaaggtaacctcctccgccacattaaaCTG | 46L_47H_49P_50I_52G_53F_54G_55C_67L | 2.35422958 |
| 3107 | 315 | TTCTGCTGTGAGATCTGTGGGGCATCATTTactcagaaaggtaacctcctccgccacattaaaCTG | 46F_47C_49E_50I_52G_53A_54S_55F_67L | 2.353966965 |
| 3108 | 3596 | TTCTCTTGTCCTGTTTGTGGGGCAACATTTactcagaaaggtaacctcctccgccacattaaaCTG | 46F_47S_49P_50V_52G_53A_54T_55F_67L | 2.353536052 |
| 3109 | 6807 | CTTTGGTGTCCTATATGCGGCTTTATTTGCactcagaaaggtaacctcctccgccacattaaaCTG | 46L_47W_49P_50I_52G_53F_54I_55C_67L | 2.352724939 |
| 3110 | 3785 | TTCACTTGTCCCATCTGTGGGGCAGGCTTTactcagaaaggtaacctcctccgccacattaaaCTG | 46F_47T_49P_50I_52G_53A_54G_55F_67L | 2.351901922 |
| 3111 | 3589 | TTCTCTTGTCCCGTGTGTGGGGCACTGTTTactcagaaaggtaacctcctccgccacattaaaCTG | 46F_47S_49P_50V_52G_53A_54L_55F_67L | 2.351651334 |
| 3112 | 1060 | TTCTTCTGTGAGGTTTGTGGGGCAGCTTTTactcagaaaggtaacctcctccgccacattaaaCTG | 46F_47F_49E_50V_52G_53A_54A_55F_67L | 2.350954509 |
| 3113 | 6013 | TTCACTTGTCCAATCTGCGGTCATCAATTCactcagaaaggtaacctcctccgccacattaaaCTG | 46F_47T_49P_50I_52G_53H_54Q_55F_67L | 2.346642257 |
| 3114 | 791 | TTCGAGTGTGAAATTTGTGGGGCAAACTTTactcagaaaggtaacctcctccgccacattaaaCTG | 46F_47E_49E_50I_52G_53A_54N_55F_67L | 2.346552582 |
| 3115 | 3900 | TTCGTGTGTGAAATCTGTGGGGCAGCATTTactcagaaaggtaacctcctccgccacattaaaCTG | 46F_47V_49E_50I_52G_53A_54A_55F_67L | 2.346000654 |
| 3116 | 1665 | TTCCATTGTCCGGTGTGTGGGGCAGGCTTTactcagaaaggtaacctcctccgccacattaaaCTG | 46F_47H_49P_50V_52G_53A_54G_55F_67L | 2.345807171 |
| 3117 | 1759 | TTCATTTGTGAGATCTGTGGGGCATACTTTactcagaaaggtaacctcctccgccacattaaaCTG | 46F_47I_49E_50I_52G_53A_54Y_55F_67L | 2.343815101 |
| 3118 | 3110 | TTCCAGTGTCCAGTCTGTGGGGCAATGTTTactcagaaaggtaacctcctccgccacattaaaCTG | 46F_47Q_49P_50V_52G_53A_54M_55F_67L | 2.342943522 |
| 3119 | 6559 | CTGTTTTGTCCAATCTGCGGGTTCTATTGCactcagaaaggtaacctcctccgccacattaaaCTG | 46L_47F_49P_50I_52G_53F_54Y_55C_67L | 2.342902984 |
| 3120 | 3120 | TTCAGATGTGACATTTGTGGGGCAGCTTTTactcagaaaggtaacctcctccgccacattaaaCTG | 46F_47R_49D_50I_52G_53A_54A_55F_67L | 2.342787311 |
| 3121 | 4150 | TTCTGGTGTGAGATATGTGGGGCAATGTTTactcagaaaggtaacctcctccgccacattaaaCTG | 46F_47W_49E_50I_52G_53A_54M_55F_67L | 2.341867004 |
| 3122 | 3112 | TTCCAATGTCCCGTTTGTGGGGCACCTTTTactcagaaaggtaacctcctccgccacattaaaCTG | 46F_47Q_49P_50V_52G_53A_54P_55F_67L | 2.340184107 |
| 3123 | 7856 | CTGCAGTGCGAGATTTGTGGCTTCACATGCactcagaaaggtaacctcctccgccacattaaaCTG | 46L_47Q_49E_50I_52G_53F_54T_55C_67L | 2.339488568 |
| 3124 | 7756 | CTCATCTGCGAGATCTGTGGATTCACCTGTactcagaaaggtaacctcctccgccacattaaaCTG | 46L_47I_49E_50I_52G_53F_54T_55C_67L | 2.339351952 |
| 3125 | 2940 | TTCCAATGTGAAATCTGTGGGGCAGCATTTactcagaaaggtaacctcctccgccacattaaaCTG | 46F_47Q_49E_50I_52G_53A_54A_55F_67L | 2.33862196 |
| 3126 | 6578 | CTGGGGTGCCCAATCTGTGGCTTTTGGTGCactcagaaaggtaacctcctccgccacattaaaCTG | 46L_47G_49P_50I_52G_53F_54W_55C_67L | 2.337737043 |
| 3127 | 2957 | TTCCAGTGTGAAATTTGTGGGGCAGTTTTTactcagaaaggtaacctcctccgccacattaaaCTG | 46F_47Q_49E_50I_52G_53A_54V_55F_67L | 2.337193009 |
| 3128 | 3556 | TTCTCATGTCCCATCTGTGGGGCAACTTTTactcagaaaggtaacctcctccgccacattaaaCTG | 46F_47S_49P_50I_52G_53A_54T_55F_67L | 2.336341255 |
| 3129 | 4555 | TTCTACTGTCCCGTGTGTGGGGCAAGCTTTactcagaaaggtaacctcctccgccacattaaaCTG | 46F_47Y_49P_50V_52G_53A_54S_55F_67L | 2.335495261 |
| 3130 | 4260 | TTCTGGTGTCCCATATGTGGGGCAGCTTTTactcagaaaggtaacctcctccgccacattaaaCTG | 46F_47W_49P_50I_52G_53A_54A_55F_67L | 2.334839727 |
| 3131 | 3660 | TTCACGTGTGAAATTTGTGGGGCAGCTTTTactcagaaaggtaacctcctccgccacattaaaCTG | 46F_47T_49E_50I_52G_53A_54A_55F_67L | 2.333752776 |
| 3132 | 3076 | TTCCAGTGTCCAATATGTGGGGCAACCTTTactcagaaaggtaacctcctccgccacattaaaCTG | 46F_47Q_49P_50I_52G_53A_54T_55F_67L | 2.333032691 |
| 3133 | 2272 | TTCCTCTGTGAAGTCTGTGGGGCACCCTTTactcagaaaggtaacctcctccgccacattaaaCTG | 46F_47L_49E_50V_52G_53A_54P_55F_67L | 2.332133058 |
| 3134 | 3225 | TTCAGATGTGAGGTCTGTGGGGCAGGCTTTactcagaaaggtaacctcctccgccacattaaaCTG | 46F_47R_49E_50V_52G_53A_54G_55F_67L | 2.330040575 |
| 3135 | 3787 | TTCACGTGTCCAATCTGTGGGGCAATCTTTactcagaaaggtaacctcctccgccacattaaaCTG | 46F_47T_49P_50I_52G_53A_54I_55F_67L | 2.33001957 |
| 3136 | 3272 | TTCCGGTGTAACCAGTGTGGGGCACCCTTTactcagaaaggtaacctcctccgccacattaaaCTG | 46F_47R_49N_50Q_52G_53A_54P_55F_67L | 2.328544816 |
| 3137 | 1797 | TTCATCTGTGAGGTGTGTGGGGCAGTGTTTactcagaaaggtaacctcctccgccacattaaaCTG | 46F_47I_49E_50V_52G_53A_54V_55F_67L | 2.328527245 |
| 3138 | 6553 | TTATTCTGCCCTATCTGTGGCTTTCAGTGCactcagaaaggtaacctcctccgccacattaaaCTG | 46L_47F_49P_50I_52G_53F_54Q_55C_67L | 2.328519648 |
| 3139 | 7946 | CTGTGGTGCGAAATTTGCGGGTTTCATTGCactcagaaaggtaacctcctccgccacattaaaCTG | 46L_47W_49E_50I_52G_53F_54H_55C_67L | 2.328127406 |
| 3140 | 431 | TTCTGTTGTCCCATCTGTGGGGCAAATTTTactcagaaaggtaacctcctccgccacattaaaCTG | 46F_47C_49P_50I_52G_53A_54N_55F_67L | 2.327393804 |
| 3141 | 6725 | TTGCGCTGTCCCATATGCGGCTTCGGCTGCactcagaaaggtaacctcctccgccacattaaaCTG | 46L_47R_49P_50I_52G_53F_54G_55C_67L | 2.326947547 |
| 3142 | 1021 | TTCTTTTGTGAAATCTGTGGGGCATGCTTTactcagaaaggtaacctcctccgccacattaaaCTG | 46F_47F_49E_50I_52G_53A_54C_55F_67L | 2.326545279 |
| 3143 | 471 | TTCTGCTGTCCGGTGTGTGGGGCAAATTTTactcagaaaggtaacctcctccgccacattaaaCTG | 46F_47C_49P_50V_52G_53A_54N_55F_67L | 2.323319334 |
| 3144 | 902 | TTCGAATGTCCCATATGTGGGGCAGATTTTactcagaaaggtaacctcctccgccacattaaaCTG | 46F_47E_49P_50I_52G_53A_54D_55F_67L | 2.322895012 |
| 3145 | 1385 | TTCGGGTGTCCAATCTGTGGGGCAGGCTTTactcagaaaggtaacctcctccgccacattaaaCTG | 46F_47G_49P_50I_52G_53A_54G_55F_67L | 2.320711124 |
| 3146 | 940 | TTCGAATGTCCTGTATGTGGGGCAGCATTTactcagaaaggtaacctcctccgccacattaaaCTG | 46F_47E_49P_50V_52G_53A_54A_55F_67L | 2.320381742 |
| 3147 | 1692 | TTCATATGTGATATCTGTGGGGCACCATTTactcagaaaggtaacctcctccgccacattaaaCTG | 46F_47I_49D_50I_52G_53A_54P_55F_67L | 2.318714071 |
| 3148 | 1425 | TTCGGGTGTCCTGTTTGTGGGGCAGGCTTTactcagaaaggtaacctcctccgccacattaaaCTG | 46F_47G_49P_50V_52G_53A_54G_55F_67L | 2.318511149 |
| 3149 | 4551 | TTCTACTGTCCAGTCTGTGGGGCAAATTTTactcagaaaggtaacctcctccgccacattaaaCTG | 46F_47Y_49P_50V_52G_53A_54N_55F_67L | 2.317825889 |
| 3150 | 1812 | TTCATCTGTAATATTTGTGGGGCACCCTTTactcagaaaggtaacctcctccgccacattaaaCTG | 46F_47I_49N_50I_52G_53A_54P_55F_67L | 2.317722987 |
| 3151 | 3793 | TTCACCTGTCCAATCTGTGGGGCACAGTTTactcagaaaggtaacctcctccgccacattaaaCTG | 46F_47T_49P_50I_52G_53A_54Q_55F_67L | 2.316412684 |
| 3152 | 6809 | CTTTGGTGCCCCATCTGTGGTTTTCTTTGTactcagaaaggtaacctcctccgccacattaaaCTG | 46L_47W_49P_50I_52G_53F_54L_55C_67L | 2.315009791 |
| 3153 | 1035 | TTCTTTTGTGAGATCTGTGGGGCAAGCTTTactcagaaaggtaacctcctccgccacattaaaCTG | 46F_47F_49E_50I_52G_53A_54S_55F_67L | 2.314279588 |
| 3154 | 6814 | CTTTGGTGCCCAATATGTGGCTTCCGGTGCactcagaaaggtaacctcctccgccacattaaaCTG | 46L_47W_49P_50I_52G_53F_54R_55C_67L | 2.313090391 |
| 3155 | 3477 | TTCTCTTGTGAGGTTTGTGGGGCAGTGTTTactcagaaaggtaacctcctccgccacattaaaCTG | 46F_47S_49E_50V_52G_53A_54V_55F_67L | 2.310904022 |
| 3156 | 1780 | TTCATATGTGAGGTCTGTGGGGCAGCGTTTactcagaaaggtaacctcctccgccacattaaaCTG | 46F_47I_49E_50V_52G_53A_54A_55F_67L | 2.309599733 |
| 3157 | 6564 | TTAGGCTGTCCGATTTGTGGCTTCTTTTGCactcagaaaggtaacctcctccgccacattaaaCTG | 46L_47G_49P_50I_52G_53F_54F_55C_67L | 2.309132436 |
| 3158 | 2621 | TTCATGTGTCCGGTGTGTGGGGCATGTTTTactcagaaaggtaacctcctccgccacattaaaCTG | 46F_47M_49P_50V_52G_53A_54C_55F_67L | 2.307843328 |
| 3159 | 7940 | TTATGGTGTGAGATCTGTGGCTTTGCCTGTactcagaaaggtaacctcctccgccacattaaaCTG | 46L_47W_49E_50I_52G_53F_54A_55C_67L | 2.306452305 |
| 3160 | 4546 | TTCTATTGTCCTGTGTGTGGGGCACACTTTactcagaaaggtaacctcctccgccacattaaaCTG | 46F_47Y_49P_50V_52G_53A_54H_55F_67L | 2.305670559 |
| 3161 | 1733 | TTCATCTGTGACGTCTGTGGGGCACAATTTactcagaaaggtaacctcctccgccacattaaaCTG | 46F_47I_49D_50V_52G_53A_54Q_55F_67L | 2.305481399 |
| 3162 | 3064 | TTCCAATGTCCCATTTGTGGGGCATTCTTTactcagaaaggtaacctcctccgccacattaaaCTG | 46F_47Q_49P_50I_52G_53A_54F_55F_67L | 2.304860513 |
| 3163 | 4032 | TTCGTGTGTCCTATCTGTGGGGCACCTTTTactcagaaaggtaacctcctccgccacattaaaCTG | 46F_47V_49P_50I_52G_53A_54P_55F_67L | 2.304714902 |
| 3164 | 3836 | TTCACGTGTCCCGTCTGTGGGGCAACCTTTactcagaaaggtaacctcctccgccacattaaaCTG | 46F_47T_49P_50V_52G_53A_54T_55F_67L | 2.300415935 |
| 3165 | 671 | TTCGATTGTCCAATCTGTGGGGCAAACTTTactcagaaaggtaacctcctccgccacattaaaCTG | 46F_47D_49P_50I_52G_53A_54N_55F_67L | 2.299760304 |
| 3166 | 3940 | TTCGTCTGTGAAGTGTGTGGGGCAGCATTTactcagaaaggtaacctcctccgccacattaaaCTG | 46F_47V_49E_50V_52G_53A_54A_55F_67L | 2.299213263 |
| 3167 | 1750 | TTCATTTGTGAGATCTGTGGGGCAATGTTTactcagaaaggtaacctcctccgccacattaaaCTG | 46F_47I_49E_50I_52G_53A_54M_55F_67L | 2.297677235 |
| 3168 | 3103 | TTCCAGTGTCCCGTATGTGGGGCAGAGTTTactcagaaaggtaacctcctccgccacattaaaCTG | 46F_47Q_49P_50V_52G_53A_54E_55F_67L | 2.296756825 |
| 3169 | 2639 | TTCATGTGTCCCGTGTGTGGGGCATATTTTactcagaaaggtaacctcctccgccacattaaaCTG | 46F_47M_49P_50V_52G_53A_54Y_55F_67L | 2.296522057 |
| 3170 | 6352 | TTCAGGTGTCCAATATGTGGGCGGCCGTTCactcagaaaggtaacctcctccgccacattaaaCTG | 46F_47R_49P_50I_52G_53R_54P_55F_67L | 2.294635924 |
| 3171 | 1319 | TTCGGATGTGAGGTGTGTGGGGCATATTTTactcagaaaggtaacctcctccgccacattaaaCTG | 46F_47G_49E_50V_52G_53A_54Y_55F_67L | 2.291035414 |
| 3172 | 300 | TTCTGCTGTGAAATCTGTGGGGCAGCCTTTactcagaaaggtaacctcctccgccacattaaaCTG | 46F_47C_49E_50I_52G_53A_54A_55F_67L | 2.288056027 |
| 3173 | 6837 | CTATACTGCCCGATATGCGGGTTTGTCTGTactcagaaaggtaacctcctccgccacattaaaCTG | 46L_47Y_49P_50I_52G_53F_54V_55C_67L | 2.286563829 |
| 3174 | 3796 | TTCACATGTCCAATCTGTGGGGCAACCTTTactcagaaaggtaacctcctccgccacattaaaCTG | 46F_47T_49P_50I_52G_53A_54T_55F_67L | 2.284938256 |
| 3175 | 4543 | TTCTATTGTCCCGTGTGTGGGGCAGAGTTTactcagaaaggtaacctcctccgccacattaaaCTG | 46F_47Y_49P_50V_52G_53A_54E_55F_67L | 2.284632566 |
| 3176 | 4275 | TTCTGGTGTCCGATCTGTGGGGCAAGTTTTactcagaaaggtaacctcctccgccacattaaaCTG | 46F_47W_49P_50I_52G_53A_54S_55F_67L | 2.283604954 |
| 3177 | 3838 | TTCACCTGTCCAGTTTGTGGGGCATGGTTTactcagaaaggtaacctcctccgccacattaaaCTG | 46F_47T_49P_50V_52G_53A_54W_55F_67L | 2.283533487 |
| 3178 | 1032 | TTCTTTTGTGAAATCTGTGGGGCACCCTTTactcagaaaggtaacctcctccgccacattaaaCTG | 46F_47F_49E_50I_52G_53A_54P_55F_67L | 2.283488333 |
| 3179 | 3901 | TTCGTGTGTGAGATATGTGGGGCATGCTTTactcagaaaggtaacctcctccgccacattaaaCTG | 46F_47V_49E_50I_52G_53A_54C_55F_67L | 2.283146313 |
| 3180 | 3665 | TTCACGTGTGAGATATGTGGGGCAGGATTTactcagaaaggtaacctcctccgccacattaaaCTG | 46F_47T_49E_50I_52G_53A_54G_55F_67L | 2.282835312 |
| 3181 | 6494 | CTGTGCTGTCCTATATGCGGATTTAGGTGCactcagaaaggtaacctcctccgccacattaaaCTG | 46L_47C_49P_50I_52G_53F_54R_55C_67L | 2.281600493 |
| 3182 | 6590 | CTCCATTGTCCGATTTGTGGCTTCATGTGTactcagaaaggtaacctcctccgccacattaaaCTG | 46L_47H_49P_50I_52G_53F_54M_55C_67L | 2.281105073 |
| 3183 | 3305 | TTCAGATGTCCCATTTGTGGGGCAGGCTTTactcagaaaggtaacctcctccgccacattaaaCTG | 46F_47R_49P_50I_52G_53A_54G_55F_67L | 2.2808658 |
| 3184 | 1620 | TTCCATTGTCCAATATGTGGGGCAGCCTTTactcagaaaggtaacctcctccgccacattaaaCTG | 46F_47H_49P_50I_52G_53A_54A_55F_67L | 2.28021074 |
| 3185 | 1141 | TTCTTTTGTCCGATTTGTGGGGCATGCTTTactcagaaaggtaacctcctccgccacattaaaCTG | 46F_47F_49P_50I_52G_53A_54C_55F_67L | 2.279209764 |
| 3186 | 4556 | TTCTATTGTCCGGTGTGTGGGGCAACTTTTactcagaaaggtaacctcctccgccacattaaaCTG | 46F_47Y_49P_50V_52G_53A_54T_55F_67L | 2.278362264 |
| 3187 | 4151 | TTCTGGTGTGAGATATGTGGGGCAAATTTTactcagaaaggtaacctcctccgccacattaaaCTG | 46F_47W_49E_50I_52G_53A_54N_55F_67L | 2.276816581 |
| 3188 | 3713 | TTCACCTGTGAGGTGTGTGGGGCACAATTTactcagaaaggtaacctcctccgccacattaaaCTG | 46F_47T_49E_50V_52G_53A_54Q_55F_67L | 2.272760038 |
| 3189 | 1813 | TTCATATGTAACATATGTGGGGCACAGTTTactcagaaaggtaacctcctccgccacattaaaCTG | 46F_47I_49N_50I_52G_53A_54Q_55F_67L | 2.272755674 |
| 3190 | 6770 | CTCACCTGTCCTATTTGTGGGTTCATGTGCactcagaaaggtaacctcctccgccacattaaaCTG | 46L_47T_49P_50I_52G_53F_54M_55C_67L | 2.2723719 |
| 3191 | 2944 | TTCCAGTGTGAAATTTGTGGGGCATTTTTTactcagaaaggtaacctcctccgccacattaaaCTG | 46F_47Q_49E_50I_52G_53A_54F_55F_67L | 2.271816135 |
| 3192 | 1195 | TTCTTCTGTCCCGTTTGTGGGGCATCCTTTactcagaaaggtaacctcctccgccacattaaaCTG | 46F_47F_49P_50V_52G_53A_54S_55F_67L | 2.270264332 |
| 3193 | 1401 | TTCGGCTGTCCCCAGTGTGGGGCATGTTTTactcagaaaggtaacctcctccgccacattaaaCTG | 46F_47G_49P_50Q_52G_53A_54C_55F_67L | 2.2686542 |
| 3194 | 7735 | CTGCACTGTGAAATATGCGGCTTTTCATGCactcagaaaggtaacctcctccgccacattaaaCTG | 46L_47H_49E_50I_52G_53F_54S_55C_67L | 2.268297723 |
| 3195 | 6464 | TTGGCGTGTCCCATCTGTGGCTTTTTTTGCactcagaaaggtaacctcctccgccacattaaaCTG | 46L_47A_49P_50I_52G_53F_54F_55C_67L | 2.267994569 |
| 3196 | 3475 | TTCAGCTGTGAAGTGTGTGGGGCAAGTTTTactcagaaaggtaacctcctccgccacattaaaCTG | 46F_47S_49E_50V_52G_53A_54S_55F_67L | 2.267741909 |
| 3197 | 2517 | TTCATGTGTGAAGTCTGTGGGGCAGTTTTTactcagaaaggtaacctcctccgccacattaaaCTG | 46F_47M_49E_50V_52G_53A_54V_55F_67L | 2.267337176 |
| 3198 | 6780 | CTCGTTTGCCCAATTTGTGGGTTTGCTTGTactcagaaaggtaacctcctccgccacattaaaCTG | 46L_47V_49P_50I_52G_53F_54A_55C_67L | 2.263218017 |
| 3199 | 4434 | TTCTACTGTGAGGTCTGTGGGGCAAGGTTTactcagaaaggtaacctcctccgccacattaaaCTG | 46F_47Y_49E_50V_52G_53A_54R_55F_67L | 2.262396744 |
| 3200 | 3547 | TTCTCCTGTCCTATATGTGGGGCAATCTTTactcagaaaggtaacctcctccgccacattaaaCTG | 46F_47S_49P_50I_52G_53A_54I_55F_67L | 2.261546442 |
| 3201 | 7933 | CTGGTGTGTGAAATATGCGGCTTCCAATGTactcagaaaggtaacctcctccgccacattaaaCTG | 46L_47V_49E_50I_52G_53F_54Q_55C_67L | 2.260582138 |
| 3202 | 7957 | CTGTGGTGCGAGATTTGTGGATTTGTATGCactcagaaaggtaacctcctccgccacattaaaCTG | 46L_47W_49E_50I_52G_53F_54V_55C_67L | 2.260176903 |
| 3203 | 6129 | TTCGACTGCCCAATCTGTGGCCGGCTGTTCactcagaaaggtaacctcctccgccacattaaaCTG | 46F_47D_49P_50I_52G_53R_54L_55F_67L | 2.257667799 |
| 3204 | 3212 | TTCCGGTGTGAACAGTGTGGGGCACCATTTactcagaaaggtaacctcctccgccacattaaaCTG | 46F_47R_49E_50Q_52G_53A_54P_55F_67L | 2.25491705 |
| 3205 | 3719 | TTCACCTGTGAGGTGTGTGGGGCATATTTTactcagaaaggtaacctcctccgccacattaaaCTG | 46F_47T_49E_50V_52G_53A_54Y_55F_67L | 2.252022137 |
| 3206 | 6773 | CTGACGTGCCCTATTTGTGGCTTCCAGTGTactcagaaaggtaacctcctccgccacattaaaCTG | 46L_47T_49P_50I_52G_53F_54Q_55C_67L | 2.251639019 |
| 3207 | 4504 | TTCTACTGTCCAATCTGTGGGGCATTTTTTactcagaaaggtaacctcctccgccacattaaaCTG | 46F_47Y_49P_50I_52G_53A_54F_55F_67L | 2.25109281 |
| 3208 | 3345 | TTCAGATGTCCTGTCTGTGGGGCAGGCTTTactcagaaaggtaacctcctccgccacattaaaCTG | 46F_47R_49P_50V_52G_53A_54G_55F_67L | 2.249651997 |
| 3209 | 355 | TTCTGTTGTGAGGTCTGTGGGGCAAGTTTTactcagaaaggtaacctcctccgccacattaaaCTG | 46F_47C_49E_50V_52G_53A_54S_55F_67L | 2.249390787 |
| 3210 | 3673 | TTCACCTGTGAGATTTGTGGGGCACAATTTactcagaaaggtaacctcctccgccacattaaaCTG | 46F_47T_49E_50I_52G_53A_54Q_55F_67L | 2.246716943 |
| 3211 | 7959 | TTGTGGTGCGAGATCTGCGGCTTCTATTGCactcagaaaggtaacctcctccgccacattaaaCTG | 46L_47W_49E_50I_52G_53F_54Y_55C_67L | 2.245279335 |
| 3212 | 6771 | CTCACCTGTCCCATTTGTGGCTTTAACTGTactcagaaaggtaacctcctccgccacattaaaCTG | 46L_47T_49P_50I_52G_53F_54N_55C_67L | 2.24491874 |
| 3213 | 3420 | TTCAGCTGTGAGATATGTGGGGCAGCGTTTactcagaaaggtaacctcctccgccacattaaaCTG | 46F_47S_49E_50I_52G_53A_54A_55F_67L | 2.243916721 |
| 3214 | 6781 | CTCGTCTGCCCTATATGCGGCTTCTGCTGCactcagaaaggtaacctcctccgccacattaaaCTG | 46L_47V_49P_50I_52G_53F_54C_55C_67L | 2.242152181 |
| 3215 | 3831 | TTCACATGTCCCGTGTGTGGGGCAAACTTTactcagaaaggtaacctcctccgccacattaaaCTG | 46F_47T_49P_50V_52G_53A_54N_55F_67L | 2.24185221 |
| 3216 | 4544 | TTCTACTGTCCCGTCTGTGGGGCATTCTTTactcagaaaggtaacctcctccgccacattaaaCTG | 46F_47Y_49P_50V_52G_53A_54F_55F_67L | 2.238896929 |
| 3217 | 3952 | TTCGTGTGTGAGGTGTGTGGGGCACCTTTTactcagaaaggtaacctcctccgccacattaaaCTG | 46F_47V_49E_50V_52G_53A_54P_55F_67L | 2.238440715 |
| 3218 | 4429 | TTCTATTGTGAAGTGTGTGGGGCACTGTTTactcagaaaggtaacctcctccgccacattaaaCTG | 46F_47Y_49E_50V_52G_53A_54L_55F_67L | 2.235804236 |
| 3219 | 4190 | TTCTGGTGTGAAGTGTGTGGGGCAATGTTTactcagaaaggtaacctcctccgccacattaaaCTG | 46F_47W_49E_50V_52G_53A_54M_55F_67L | 2.234269717 |
| 3220 | 7801 | CTGATGTGCGAGATTTGCGGGTTCTGCTGCactcagaaaggtaacctcctccgccacattaaaCTG | 46L_47M_49E_50I_52G_53F_54C_55C_67L | 2.234100514 |
| 3221 | 1747 | TTCATTTGTGAGATATGTGGGGCAATCTTTactcagaaaggtaacctcctccgccacattaaaCTG | 46F_47I_49E_50I_52G_53A_54I_55F_67L | 2.23360817 |
| 3222 | 912 | TTCGAGTGTCCTATCTGTGGGGCACCTTTTactcagaaaggtaacctcctccgccacattaaaCTG | 46F_47E_49P_50I_52G_53A_54P_55F_67L | 2.23221226 |
| 3223 | 2981 | TTCCAGTGTGAAGTGTGTGGGGCATGCTTTactcagaaaggtaacctcctccgccacattaaaCTG | 46F_47Q_49E_50V_52G_53A_54C_55F_67L | 2.231768604 |
| 3224 | 1192 | TTCTTTTGTCCAGTGTGTGGGGCACCTTTTactcagaaaggtaacctcctccgccacattaaaCTG | 46F_47F_49P_50V_52G_53A_54P_55F_67L | 2.231124912 |
| 3225 | 2955 | TTCCAATGTGAGATCTGTGGGGCAAGCTTTactcagaaaggtaacctcctccgccacattaaaCTG | 46F_47Q_49E_50I_52G_53A_54S_55F_67L | 2.227418511 |
| 3226 | 7876 | CTCAGATGCGAAATCTGTGGGTTCACATGTactcagaaaggtaacctcctccgccacattaaaCTG | 46L_47R_49E_50I_52G_53F_54T_55C_67L | 2.226648843 |
| 3227 | 3469 | TTCTCTTGTGAGGTTTGTGGGGCATTGTTTactcagaaaggtaacctcctccgccacattaaaCTG | 46F_47S_49E_50V_52G_53A_54L_55F_67L | 2.225739183 |
| 3228 | 1799 | TTCATCTGTGAGGTGTGTGGGGCATATTTTactcagaaaggtaacctcctccgccacattaaaCTG | 46F_47I_49E_50V_52G_53A_54Y_55F_67L | 2.224673669 |
| 3229 | 430 | TTCTGCTGTCCCATCTGTGGGGCAATGTTTactcagaaaggtaacctcctccgccacattaaaCTG | 46F_47C_49P_50I_52G_53A_54M_55F_67L | 2.223956734 |
| 3230 | 6764 | CTAACATGCCCCATATGTGGATTCTTCTGCactcagaaaggtaacctcctccgccacattaaaCTG | 46L_47T_49P_50I_52G_53F_54F_55C_67L | 2.222194397 |
| 3231 | 661 | TTCGATTGTCCAATTTGTGGGGCATGTTTTactcagaaaggtaacctcctccgccacattaaaCTG | 46F_47D_49P_50I_52G_53A_54C_55F_67L | 2.221255424 |
| 3232 | 6597 | CTGCACTGCCCGATTTGCGGCTTCGTGTGCactcagaaaggtaacctcctccgccacattaaaCTG | 46L_47H_49P_50I_52G_53F_54V_55C_67L | 2.22122898 |
| 3233 | 1430 | TTCGGATGTCCGGTTTGTGGGGCAATGTTTactcagaaaggtaacctcctccgccacattaaaCTG | 46F_47G_49P_50V_52G_53A_54M_55F_67L | 2.219049354 |
| 3234 | 6796 | TTAGTCTGCCCTATCTGCGGTTTTACCTGCactcagaaaggtaacctcctccgccacattaaaCTG | 46L_47V_49P_50I_52G_53F_54T_55C_67L | 2.218506484 |
| 3235 | 4545 | TTCTACTGTCCCGTCTGTGGGGCAGGATTTactcagaaaggtaacctcctccgccacattaaaCTG | 46F_47Y_49P_50V_52G_53A_54G_55F_67L | 2.21817428 |
| 3236 | 1900 | TTCATATGTCCCGTATGTGGGGCAGCCTTTactcagaaaggtaacctcctccgccacattaaaCTG | 46F_47I_49P_50V_52G_53A_54A_55F_67L | 2.218103911 |
| 3237 | 4549 | TTCTATTGTCCGGTGTGTGGGGCATTGTTTactcagaaaggtaacctcctccgccacattaaaCTG | 46F_47Y_49P_50V_52G_53A_54L_55F_67L | 2.218100474 |
| 3238 | 7492 | TTCCGGTGTGAGATCTGCGGAAGGCCTTTTactcagaaaggtaacctcctccgccacattaaaCTG | 46F_47R_49E_50I_52G_53R_54P_55F_67L | 2.217638195 |
| 3239 | 2980 | TTCCAATGTGAAGTTTGTGGGGCAGCCTTTactcagaaaggtaacctcctccgccacattaaaCTG | 46F_47Q_49E_50V_52G_53A_54A_55F_67L | 2.217542095 |
| 3240 | 2535 | TTCATGTGTAATATTTGTGGGGCAAGTTTTactcagaaaggtaacctcctccgccacattaaaCTG | 46F_47M_49N_50I_52G_53A_54S_55F_67L | 2.216999199 |
| 3241 | 1790 | TTCATTTGTGAAGTGTGTGGGGCAATGTTTactcagaaaggtaacctcctccgccacattaaaCTG | 46F_47I_49E_50V_52G_53A_54M_55F_67L | 2.216443789 |
| 3242 | 7819 | CTTATGTGTGAGATATGCGGCTTTTACTGTactcagaaaggtaacctcctccgccacattaaaCTG | 46L_47M_49E_50I_52G_53F_54Y_55C_67L | 2.216115717 |
| 3243 | 2413 | TTCATGTGTGACATTTGTGGGGCACAGTTTactcagaaaggtaacctcctccgccacattaaaCTG | 46F_47M_49D_50I_52G_53A_54Q_55F_67L | 2.214649605 |
| 3244 | 7810 | CTCATGTGCGAGATATGCGGATTTATGTGTactcagaaaggtaacctcctccgccacattaaaCTG | 46L_47M_49E_50I_52G_53F_54M_55C_67L | 2.214024971 |
| 3245 | 3732 | TTCACATGTAATATCTGTGGGGCACCCTTTactcagaaaggtaacctcctccgccacattaaaCTG | 46F_47T_49N_50I_52G_53A_54P_55F_67L | 2.213582682 |
| 3246 | 6670 | CTGATGTGTCCAATCTGTGGCTTTATGTGTactcagaaaggtaacctcctccgccacattaaaCTG | 46L_47M_49P_50I_52G_53F_54M_55C_67L | 2.212942568 |
| 3247 | 1860 | TTCATTTGTCCTATATGTGGGGCAGCCTTTactcagaaaggtaacctcctccgccacattaaaCTG | 46F_47I_49P_50I_52G_53A_54A_55F_67L | 2.212403179 |
| 3248 | 3007 | TTCCAGTGTAACATATGTGGGGCAATCTTTactcagaaaggtaacctcctccgccacattaaaCTG | 46F_47Q_49N_50I_52G_53A_54I_55F_67L | 2.211592612 |
| 3249 | 1159 | TTCTTTTGTCCGATTTGTGGGGCATACTTTactcagaaaggtaacctcctccgccacattaaaCTG | 46F_47F_49P_50I_52G_53A_54Y_55F_67L | 2.210380575 |
| 3250 | 2993 | TTCCAGTGTGAGGTTTGTGGGGCACAGTTTactcagaaaggtaacctcctccgccacattaaaCTG | 46F_47Q_49E_50V_52G_53A_54Q_55F_67L | 2.209881205 |
| 3251 | 955 | TTCGAATGTCCCGTGTGTGGGGCAAGCTTTactcagaaaggtaacctcctccgccacattaaaCTG | 46F_47E_49P_50V_52G_53A_54S_55F_67L | 2.203739439 |
| 3252 | 6758 | CTCTCGTGCCCTATATGTGGATTTTGGTGCactcagaaaggtaacctcctccgccacattaaaCTG | 46L_47S_49P_50I_52G_53F_54W_55C_67L | 2.201886962 |
| 3253 | 2597 | TTCATGTGTCCAATCTGTGGGGCAGTGTTTactcagaaaggtaacctcctccgccacattaaaCTG | 46F_47M_49P_50I_52G_53A_54V_55F_67L | 2.20030169 |
| 3254 | 7967 | CTCTACTGCGAGATCTGTGGGTTCATATGCactcagaaaggtaacctcctccgccacattaaaCTG | 46L_47Y_49E_50I_52G_53F_54I_55C_67L | 2.199429389 |
| 3255 | 2476 | TTCATGTGTGAGATCTGTGGGGCAACCTTTactcagaaaggtaacctcctccgccacattaaaCTG | 46F_47M_49E_50I_52G_53A_54T_55F_67L | 2.196697513 |
| 3256 | 6799 | CTGGTGTGTCCTATTTGTGGCTTTTACTGCactcagaaaggtaacctcctccgccacattaaaCTG | 46L_47V_49P_50I_52G_53F_54Y_55C_67L | 2.196010869 |
| 3257 | 1037 | TTCTTTTGTGAGATCTGTGGGGCAGTGTTTactcagaaaggtaacctcctccgccacattaaaCTG | 46F_47F_49E_50I_52G_53A_54V_55F_67L | 2.195322517 |
| 3258 | 1389 | TTCGGTTGTCCAATTTGTGGGGCACTGTTTactcagaaaggtaacctcctccgccacattaaaCTG | 46F_47G_49P_50I_52G_53A_54L_55F_67L | 2.194519408 |
| 3259 | 3071 | TTCCAGTGTCCTATTTGTGGGGCAAATTTTactcagaaaggtaacctcctccgccacattaaaCTG | 46F_47Q_49P_50I_52G_53A_54N_55F_67L | 2.194057325 |
| 3260 | 7817 | CTGATGTGTGAGATATGTGGCTTTGTGTGCactcagaaaggtaacctcctccgccacattaaaCTG | 46L_47M_49E_50I_52G_53F_54V_55C_67L | 2.192509452 |
| 3261 | 2440 | TTCATGTGTGATGTTTGTGGGGCAGCCTTTactcagaaaggtaacctcctccgccacattaaaCTG | 46F_47M_49D_50V_52G_53A_54A_55F_67L | 2.191713806 |
| 3262 | 1673 | TTCCATTGTCCAGTGTGTGGGGCACAATTTactcagaaaggtaacctcctccgccacattaaaCTG | 46F_47H_49P_50V_52G_53A_54Q_55F_67L | 2.191344162 |
| 3263 | 6805 | CTGTGGTGTCCAATCTGCGGTTTTGGCTGCactcagaaaggtaacctcctccgccacattaaaCTG | 46L_47W_49P_50I_52G_53F_54G_55C_67L | 2.189463157 |
| 3264 | 3599 | TTCTCATGTCCCGTTTGTGGGGCATATTTTactcagaaaggtaacctcctccgccacattaaaCTG | 46F_47S_49P_50V_52G_53A_54Y_55F_67L | 2.18907194 |
| 3265 | 470 | TTCTGTTGTCCAGTCTGTGGGGCAATGTTTactcagaaaggtaacctcctccgccacattaaaCTG | 46F_47C_49P_50V_52G_53A_54M_55F_67L | 2.187538693 |
| 3266 | 4035 | TTCGTCTGTCCTATCTGTGGGGCAAGTTTTactcagaaaggtaacctcctccgccacattaaaCTG | 46F_47V_49P_50I_52G_53A_54S_55F_67L | 2.186872679 |
| 3267 | 4509 | TTCTATTGTCCAATCTGTGGGGCATTGTTTactcagaaaggtaacctcctccgccacattaaaCTG | 46F_47Y_49P_50I_52G_53A_54L_55F_67L | 2.186789605 |
| 3268 | 1140 | TTCTTCTGTCCAATTTGTGGGGCAGCCTTTactcagaaaggtaacctcctccgccacattaaaCTG | 46F_47F_49P_50I_52G_53A_54A_55F_67L | 2.184884851 |
| 3269 | 4557 | TTCTATTGTCCAGTTTGTGGGGCAGTGTTTactcagaaaggtaacctcctccgccacattaaaCTG | 46F_47Y_49P_50V_52G_53A_54V_55F_67L | 2.184385398 |
| 3270 | 1155 | TTCTTCTGTCCCATCTGTGGGGCATCCTTTactcagaaaggtaacctcctccgccacattaaaCTG | 46F_47F_49P_50I_52G_53A_54S_55F_67L | 2.183467149 |
| 3271 | 4146 | TTCTGGTGTGAGATCTGTGGGGCACACTTTactcagaaaggtaacctcctccgccacattaaaCTG | 46F_47W_49E_50I_52G_53A_54H_55F_67L | 2.183012776 |
| 3272 | 7916 | CTGACCTGCGAAATCTGCGGATTTACTTGCactcagaaaggtaacctcctccgccacattaaaCTG | 46L_47T_49E_50I_52G_53F_54T_55C_67L | 2.18292761 |
| 3273 | 4424 | TTCTATTGTGAGGTCTGTGGGGCATTCTTTactcagaaaggtaacctcctccgccacattaaaCTG | 46F_47Y_49E_50V_52G_53A_54F_55F_67L | 2.17579673 |
| 3274 | 6520 | CTGGAGTGTCCCATCTGCGGGTTCGCTTGCactcagaaaggtaacctcctccgccacattaaaCTG | 46L_47E_49P_50I_52G_53F_54A_55C_67L | 2.17425789 |
| 3275 | 7974 | CTGTATTGTGAAATTTGCGGCTTTCGCTGCactcagaaaggtaacctcctccgccacattaaaCTG | 46L_47Y_49E_50I_52G_53F_54R_55C_67L | 2.174066784 |
| 3276 | 469 | TTCTGTTGTCCGGTCTGTGGGGCACTGTTTactcagaaaggtaacctcctccgccacattaaaCTG | 46F_47C_49P_50V_52G_53A_54L_55F_67L | 2.173715084 |
| 3277 | 6724 | CTCCGATGCCCTATCTGTGGTTTTTTTTGCactcagaaaggtaacctcctccgccacattaaaCTG | 46L_47R_49P_50I_52G_53F_54F_55C_67L | 2.171080197 |
| 3278 | 907 | TTCGAATGTCCCATCTGTGGGGCAATCTTTactcagaaaggtaacctcctccgccacattaaaCTG | 46F_47E_49P_50I_52G_53A_54I_55F_67L | 2.170551776 |
| 3279 | 1915 | TTCATCTGTCCAGTGTGTGGGGCATCCTTTactcagaaaggtaacctcctccgccacattaaaCTG | 46F_47I_49P_50V_52G_53A_54S_55F_67L | 2.170120711 |
| 3280 | 3555 | TTCAGTTGTCCTATCTGTGGGGCAAGCTTTactcagaaaggtaacctcctccgccacattaaaCTG | 46F_47S_49P_50I_52G_53A_54S_55F_67L | 2.167440946 |
| 3281 | 6803 | TTGTGGTGTCCTATTTGTGGGTTCGAGTGCactcagaaaggtaacctcctccgccacattaaaCTG | 46L_47W_49P_50I_52G_53F_54E_55C_67L | 2.167326492 |
| 3282 | 3559 | TTCTCCTGTCCGATCTGTGGGGCATATTTTactcagaaaggtaacctcctccgccacattaaaCTG | 46F_47S_49P_50I_52G_53A_54Y_55F_67L | 2.167139488 |
| 3283 | 1396 | TTCGGATGTCCCATCTGTGGGGCAACATTTactcagaaaggtaacctcctccgccacattaaaCTG | 46F_47G_49P_50I_52G_53A_54T_55F_67L | 2.166857072 |
| 3284 | 6678 | CTGATGTGCCCCATCTGCGGATTTTGGTGTactcagaaaggtaacctcctccgccacattaaaCTG | 46L_47M_49P_50I_52G_53F_54W_55C_67L | 2.165742856 |
| 3285 | 6765 | CTCACTTGTCCGATTTGCGGTTTCGGATGTactcagaaaggtaacctcctccgccacattaaaCTG | 46L_47T_49P_50I_52G_53F_54G_55C_67L | 2.165339099 |
| 3286 | 6832 | TTATACTGCCCCATCTGCGGATTCCCTTGTactcagaaaggtaacctcctccgccacattaaaCTG | 46L_47Y_49P_50I_52G_53F_54P_55C_67L | 2.163886066 |
| 3287 | 6533 | TTAGAATGCCCTATCTGCGGTTTCCAATGCactcagaaaggtaacctcctccgccacattaaaCTG | 46L_47E_49P_50I_52G_53F_54Q_55C_67L | 2.161189742 |
| 3288 | 432 | TTCTGCTGTCCGATCTGTGGGGCACCTTTTactcagaaaggtaacctcctccgccacattaaaCTG | 46F_47C_49P_50I_52G_53A_54P_55F_67L | 2.16067899 |
| 3289 | 6739 | CTCAGATGCCCCATCTGTGGTTTCTATTGCactcagaaaggtaacctcctccgccacattaaaCTG | 46L_47R_49P_50I_52G_53F_54Y_55C_67L | 2.157438197 |
| 3290 | 1660 | TTCCACTGTCCTGTTTGTGGGGCAGCATTTactcagaaaggtaacctcctccgccacattaaaCTG | 46F_47H_49P_50V_52G_53A_54A_55F_67L | 2.15692029 |
| 3291 | 4191 | TTCTGGTGTGAGGTTTGTGGGGCAAACTTTactcagaaaggtaacctcctccgccacattaaaCTG | 46F_47W_49E_50V_52G_53A_54N_55F_67L | 2.156432401 |
| 3292 | 1181 | TTCTTCTGTCCAGTATGTGGGGCATGCTTTactcagaaaggtaacctcctccgccacattaaaCTG | 46F_47F_49P_50V_52G_53A_54C_55F_67L | 2.156004164 |
| 3293 | 1751 | TTCATCTGTGAAATCTGTGGGGCAAATTTTactcagaaaggtaacctcctccgccacattaaaCTG | 46F_47I_49E_50I_52G_53A_54N_55F_67L | 2.15487014 |
| 3294 | 3798 | TTCACATGTCCAATTTGTGGGGCATGGTTTactcagaaaggtaacctcctccgccacattaaaCTG | 46F_47T_49P_50I_52G_53A_54W_55F_67L | 2.153530373 |
| 3295 | 6489 | CTCTGCTGTCCAATCTGTGGATTCCTGTGTactcagaaaggtaacctcctccgccacattaaaCTG | 46L_47C_49P_50I_52G_53F_54L_55C_67L | 2.153527124 |
| 3296 | 3933 | TTCGTGTGTGAACAGTGTGGGGCACAGTTTactcagaaaggtaacctcctccgccacattaaaCTG | 46F_47V_49E_50Q_52G_53A_54Q_55F_67L | 2.152346779 |
| 3297 | 1260 | TTCGGATGTGAGATCTGTGGGGCAGCGTTTactcagaaaggtaacctcctccgccacattaaaCTG | 46F_47G_49E_50I_52G_53A_54A_55F_67L | 2.151091428 |
| 3298 | 7814 | TTGATGTGCGAGATCTGTGGGTTTAGATGTactcagaaaggtaacctcctccgccacattaaaCTG | 46L_47M_49E_50I_52G_53F_54R_55C_67L | 2.148552892 |
| 3299 | 6818 | CTGTGGTGTCCCATTTGTGGTTTCTGGTGCactcagaaaggtaacctcctccgccacattaaaCTG | 46L_47W_49P_50I_52G_53F_54W_55C_67L | 2.145915935 |
| 3300 | 1199 | TTCTTTTGTCCCGTGTGTGGGGCATATTTTactcagaaaggtaacctcctccgccacattaaaCTG | 46F_47F_49P_50V_52G_53A_54Y_55F_67L | 2.145759268 |
| 3301 | 3235 | TTCCGATGTGAGGTGTGTGGGGCAAGCTTTactcagaaaggtaacctcctccgccacattaaaCTG | 46F_47R_49E_50V_52G_53A_54S_55F_67L | 2.145006292 |
| 3302 | 2467 | TTCATGTGTGAAATCTGTGGGGCAATATTTactcagaaaggtaacctcctccgccacattaaaCTG | 46F_47M_49E_50I_52G_53A_54I_55F_67L | 2.140714376 |
| 3303 | 6452 | TTCTACTGCCCAATTTGCGGCCGGCCATTTactcagaaaggtaacctcctccgccacattaaaCTG | 46F_47Y_49P_50I_52G_53R_54P_55F_67L | 2.13786653 |
| 3304 | 1277 | TTCGGATGTGAAATTTGTGGGGCAGTGTTTactcagaaaggtaacctcctccgccacattaaaCTG | 46F_47G_49E_50I_52G_53A_54V_55F_67L | 2.137793612 |
| 3305 | 3561 | TTCAGCTGTCCACAGTGTGGGGCATGCTTTactcagaaaggtaacctcctccgccacattaaaCTG | 46F_47S_49P_50Q_52G_53A_54C_55F_67L | 2.137650293 |
| 3306 | 7941 | CTATGGTGCGAAATATGCGGATTCTGTTGCactcagaaaggtaacctcctccgccacattaaaCTG | 46L_47W_49E_50I_52G_53F_54C_55C_67L | 2.136223608 |
| 3307 | 6671 | TTAATGTGCCCAATCTGCGGCTTTAATTGTactcagaaaggtaacctcctccgccacattaaaCTG | 46L_47M_49P_50I_52G_53F_54N_55C_67L | 2.13539167 |
| 3308 | 377 | TTCTGCTGTAATATTTGTGGGGCAGTATTTactcagaaaggtaacctcctccgccacattaaaCTG | 46F_47C_49N_50I_52G_53A_54V_55F_67L | 2.135104763 |
| 3309 | 6674 | CTGATGTGCCCCATATGCGGATTTCGATGCactcagaaaggtaacctcctccgccacattaaaCTG | 46L_47M_49P_50I_52G_53F_54R_55C_67L | 2.133411989 |
| 3310 | 2995 | TTCCAGTGTGAGGTCTGTGGGGCATCCTTTactcagaaaggtaacctcctccgccacattaaaCTG | 46F_47Q_49E_50V_52G_53A_54S_55F_67L | 2.133228107 |
| 3311 | 6583 | CTACACTGTCCAATCTGCGGATTTGAGTGTactcagaaaggtaacctcctccgccacattaaaCTG | 46L_47H_49P_50I_52G_53F_54E_55C_67L | 2.132401742 |
| 3312 | 6701 | CTCCAATGCCCCATTTGTGGGTTCTGCTGTactcagaaaggtaacctcctccgccacattaaaCTG | 46L_47Q_49P_50I_52G_53F_54C_55C_67L | 2.132354628 |
| 3313 | 6592 | CTTCATTGTCCAATCTGTGGGTTCCCATGCactcagaaaggtaacctcctccgccacattaaaCTG | 46L_47H_49P_50I_52G_53F_54P_55C_67L | 2.128998551 |
| 3314 | 6616 | CTGATCTGTCCCATCTGTGGGTTTACCTGCactcagaaaggtaacctcctccgccacattaaaCTG | 46L_47I_49P_50I_52G_53F_54T_55C_67L | 2.128946182 |
| 3315 | 2626 | TTCATGTGTCCCGTGTGTGGGGCACACTTTactcagaaaggtaacctcctccgccacattaaaCTG | 46F_47M_49P_50V_52G_53A_54H_55F_67L | 2.128902724 |
| 3316 | 4072 | TTCGTTTGTCCGGTCTGTGGGGCACCATTTactcagaaaggtaacctcctccgccacattaaaCTG | 46F_47V_49P_50V_52G_53A_54P_55F_67L | 2.127694317 |
| 3317 | 3160 | TTCCGTTGTGATGTATGTGGGGCAGCGTTTactcagaaaggtaacctcctccgccacattaaaCTG | 46F_47R_49D_50V_52G_53A_54A_55F_67L | 2.125167338 |
| 3318 | 7947 | TTGTGGTGTGAAATCTGCGGCTTCATTTGCactcagaaaggtaacctcctccgccacattaaaCTG | 46L_47W_49E_50I_52G_53F_54I_55C_67L | 2.12449796 |
| 3319 | 7860 | CTTCGTTGTGAAATCTGTGGCTTCGCCTGCactcagaaaggtaacctcctccgccacattaaaCTG | 46L_47R_49E_50I_52G_53F_54A_55C_67L | 2.123450255 |
| 3320 | 1066 | TTCTTCTGTGAGGTTTGTGGGGCACACTTTactcagaaaggtaacctcctcogccacattaaaCTG | 46F_47F_49E_50V_52G_53A_54H_55F_67L | 2.123227533 |
| 3321 | 6575 | CTTGGCTGCCCAATTTGCGGGTTCTCTTGTactcagaaaggtaacctcctccgccacattaaaCTG | 46L_47G_49P_50I_52G_53F_54S_55C_67L | 2.122830397 |
| 3322 | 4399 | TTCTACTGTGAGATCTGTGGGGCATACTTTactcagaaaggtaacctcctccgccacattaaaCTG | 46F_47Y_49E_50I_52G_53A_54Y_55F_67L | 2.122339385 |
| 3323 | 6757 | CTGTCCTGCCCAATATGTGGCTTCGTATGCactcagaaaggtaacctcctccgccacattaaaCTG | 46L_47S_49P_50I_52G_53F_54V_55C_67L | 2.122166585 |
| 3324 | 3152 | TTCCGCTGTGATCAGTGTGGGGCACCATTTactcagaaaggtaacctcctccgccacattaaaCTG | 46F_47R_49D_50Q_52G_53A_54P_55F_67L | 2.122115056 |
| 3325 | 4311 | TTCTGGTGTCCCGTCTGTGGGGCAAATTTTactcagaaaggtaacctcctccgccacattaaaCTG | 46F_47W_49P_50V_52G_53A_54N_55F_67L | 2.118829349 |
| 3326 | 4439 | TTCTACTGTGAAGTGTGTGGGGCATACTTTactcagaaaggtaacctcctccgccacattaaaCTG | 46F_47Y_49E_50V_52G_53A_54Y_55F_67L | 2.118745065 |
| 3327 | 4144 | TTCTGGTGTGAAATTTGTGGGGCATTCTTTactcagaaaggtaacctcctccgccacattaaaCTG | 46F_47W_49E_50I_52G_53A_54F_55F_67L | 2.117290605 |
| 3328 | 3716 | TTCACCTGTGAAGTTTGTGGGGCAACCTTTactcagaaaggtaacctcctccgccacattaaaCTG | 46F_47T_49E_50V_52G_53A_54T_55F_67L | 2.116730286 |
| 3329 | 1785 | TTCATCTGTGAGGTGTGTGGGGCAGGCTTTactcagaaaggtaacctcctccgccacattaaaCTG | 46F_47I_49E_50V_52G_53A_54G_55F_67L | 2.115308586 |
| 3330 | 6610 | TTAATTTGCCCTATATGTGGTTTTATGTGCactcagaaaggtaacctcctccgccacattaaaCTG | 46L_47I_49P_50I_52G_53F_54M_55C_67L | 2.114320268 |
| 3331 | 6613 | CTGATTTGCCCAATCTGTGGCTTTCAGTGTactcagaaaggtaacctcctccgccacattaaaCTG | 46L_47I_49P_50I_52G_53F_54Q_55C_67L | 2.114011964 |
| 3332 | 310 | TTCTGCTGTGAGATTTGTGGGGCAATGTTTactcagaaaggtaacctcctccgccacattaaaCTG | 46F_47C_49E_50I_52G_53A_54M_55F_67L | 2.112510977 |
| 3333 | 3481 | TTCAGCTGTAACATCTGTGGGGCATGTTTTactcagaaaggtaacctcctccgccacattaaaCTG | 46F_47S_49N_50I_52G_53A_54C_55F_67L | 2.112003203 |
| 3334 | 4333 | TTCTACTGTGACATTTGTGGGGCACAGTTTactcagaaaggtaacctcctccgccacattaaaCTG | 46F_47Y_49D_50I_52G_53A_54Q_55F_67L | 2.111847703 |
| 3335 | 1661 | TTCCACTGTCCTGTATGTGGGGCATGTTTTactcagaaaggtaacctcctccgccacattaaaCTG | 46F_47H_49P_50V_52G_53A_54C_55F_67L | 2.111808261 |
| 3336 | 913 | TTCGAGTGTCCTATCTGTGGGGCACAATTTactcagaaaggtaacctcctccgccacattaaaCTG | 46F_47E_49P_50I_52G_53A_54Q_55F_67L | 2.111618184 |
| 3337 | 6551 | CTGTTTTGTCCCATTTGCGGGTTCAATTGCactcagaaaggtaacctcctccgccacattaaaCTG | 46L_47F_49P_50I_52G_53F_54N_55C_67L | 2.110765669 |
| 3338 | 3435 | TTCTCGTGTGAGATCTGTGGGGCATCGTTTactcagaaaggtaacctcctccgccacattaaaCTG | 46F_47S_49E_50I_52G_53A_54S_55F_67L | 2.109764529 |
| 3339 | 3911 | TTCGTTTGTGAGATTTGTGGGGCAAATTTTactcagaaaggtaacctcctccgccacattaaaCTG | 46F_47V_49E_50I_52G_53A_54N_55F_67L | 2.107271183 |
| 3340 | 1623 | TTCCACTGTCCTATTTGTGGGGCAGAGTTTactcagaaaggtaacctcctccgccacattaaaCTG | 46F_47H_49P_50I_52G_53A_54E_55F_67L | 2.106988076 |
| 3341 | 4394 | TTCTACTGTGAAATCTGTGGGGCAAGATTTactcagaaaggtaacctcctccgccacattaaaCTG | 46F_47Y_49E_50I_52G_53A_54R_55F_67L | 2.105608941 |
| 3342 | 3720 | TTCACTTGTAATATCTGTGGGGCAGCCTTTactcagaaaggtaacctcctccgccacattaaaCTG | 46F_47T_49N_50I_52G_53A_54A_55F_67L | 2.105523272 |
| 3343 | 1337 | TTCGGCTGTAACATTTGTGGGGCAGTGTTTactcagaaaggtaacctcctccgccacattaaaCTG | 46F_47G_49N_50I_52G_53A_54V_55F_67L | 2.104669398 |
| 3344 | 3957 | TTCGTTTGTGAGGTCTGTGGGGCAGTGTTTactcagaaaggtaacctcctccgccacattaaaCTG | 46F_47V_49E_50V_52G_53A_54V_55F_67L | 2.104200014 |
| 3345 | 2415 | TTCATGTGTGACATTTGTGGGGCATCTTTTactcagaaaggtaacctcctccgccacattaaaCTG | 46F_47M_49D_50I_52G_53A_54S_55F_67L | 2.104122564 |
| 3346 | 1186 | TTCTTTTGTCCTGTCTGTGGGGCACATTTTactcagaaaggtaacctcctccgccacattaaaCTG | 46F_47F_49P_50V_52G_53A_54H_55F_67L | 2.10394624 |
| 3347 | 2292 | TTCCTGTGTAATATTTGTGGGGCACCTTTTactcagaaaggtaacctcctccgccacattaaaCTG | 46F_47L_49N_50I_52G_53A_54P_55F_67L | 2.101167437 |
| 3348 | 340 | TTCTGTTGTGAAGTGTGTGGGGCAGCCTTTactcagaaaggtaacctcctccgccacattaaaCTG | 46F_47C_49E_50V_52G_53A_54A_55F_67L | 2.099933084 |
| 3349 | 6784 | CTCGTCTGTCCTATCTGCGGATTTTTTTGCactcagaaaggtaacctcctccgccacattaaaCTG | 46L_47V_49P_50I_52G_53F_54F_55C_67L | 2.09933835 |
| 3350 | 6615 | TTGATCTGCCCAATTTGCGGCTTCTCCTGCactcagaaaggtaacctcctccgccacattaaaCTG | 46L_47I_49P_50I_52G_53F_54S_55C_67L | 2.099207208 |
| 3351 | 4506 | TTCTACTGTCCGATCTGTGGGGCACATTTTactcagaaaggtaacctcctccgccacattaaaCTG | 46F_47Y_49P_50I_52G_53A_54H_55F_67L | 2.098528921 |
| 3352 | 311 | TTCTGCTGTGAGATCTGTGGGGCAAATTTTactcagaaaggtaacctcctccgccacattaaaCTG | 46F_47C_49E_50I_52G_53A_54N_55F_67L | 2.098146635 |
| 3353 | 4505 | TTCTATTGTCCAATATGTGGGGCAGGTTTTactcagaaaggtaacctcctccgccacattaaaCTG | 46F_47Y_49P_50I_52G_53A_54G_55F_67L | 2.096782392 |
| 3354 | 953 | TTCGAGTGTCCCGTTTGTGGGGCACAATTTactcagaaaggtaacctcctccgccacattaaaCTG | 46F_47E_49P_50V_52G_53A_54Q_55F_67L | 2.094210149 |
| 3355 | 6831 | CTTTATTGCCCAATCTGTGGGTTTAATTGCactcagaaaggtaacctcctccgccacattaaaCTG | 46L_47Y_49P_50I_52G_53F_54N_55C_67L | 2.09018005 |
| 3356 | 6790 | CTTGTATGTCCAATCTGCGGCTTTATGTGCactcagaaaggtaacctcctccgccacattaaaCTG | 46L_47V_49P_50I_52G_53F_54M_55C_67L | 2.090066373 |
| 3357 | 2516 | TTCATGTGTGAAGTGTGTGGGGCAACGTTTactcagaaaggtaacctcctccgccacattaaaCTG | 46F_47M_49E_50V_52G_53A_54T_55F_67L | 2.089368023 |
| 3358 | 1404 | TTCGGATGTCCACAGTGTGGGGCATTCTTTactcagaaaggtaacctcctccgccacattaaaCTG | 46F_47G_49P_50Q_52G_53A_54F_55F_67L | 2.087617525 |
| 3359 | 3951 | TTCGTGTGTGAAGTGTGTGGGGCAAACTTTactcagaaaggtaacctcctccgccacattaaaCTG | 46F_47V_49E_50V_52G_53A_54N_55F_67L | 2.08684489 |
| 3360 | 6823 | TTGTATTGTCCGATCTGCGGGTTCGAGTGCactcagaaaggtaacctcctccgccacattaaaCTG | 46L_47Y_49P_50I_52G_53F_54E_55C_67L | 2.084394289 |
| 3361 | 3104 | TTCCAGTGTCCCGTTTGTGGGGCATTTTTTactcagaaaggtaacctcctccgccacattaaaCTG | 46F_47Q_49P_50V_52G_53A_54F_55F_67L | 2.083038866 |
| 3362 | 4021 | TTCGTGTGTCCCATCTGTGGGGCATGTTTTactcagaaaggtaacctcctccgccacattaaaCTG | 46F_47V_49P_50I_52G_53A_54C_55F_67L | 2.082721054 |
| 3363 | 4061 | TTCGTCTGTCCAGTGTGTGGGGCATGTTTTactcagaaaggtaacctcctccgccacattaaaCTG | 46F_47V_49P_50V_52G_53A_54C_55F_67L | 2.082646734 |
| 3364 | 3587 | TTCTCGTGTCCGGTCTGTGGGGCAATCTTTactcagaaaggtaacctcctccgccacattaaaCTG | 46F_47S_49P_50V_52G_53A_54I_55F_67L | 2.082280893 |
| 3365 | 3784 | TTCACCTGTCCAATCTGTGGGGCATTTTTTactcagaaaggtaacctcctccgccacattaaaCTG | 46F_47T_49P_50I_52G_53A_54F_55F_67L | 2.081183008 |
| 3366 | 4550 | TTCTATTGTCCAGTGTGTGGGGCAATGTTTactcagaaaggtaacctcctccgccacattaaaCTG | 46F_47Y_49P_50V_52G_53A_54M_55F_67L | 2.080488215 |
| 3367 | 1621 | TTCCACTGTCCTATTTGTGGGGCATGCTTTactcagaaaggtaacctcctccgccacattaaaCTG | 46F_47H_49P_50I_52G_53A_54C_55F_67L | 2.078874535 |
| 3368 | 1635 | TTCCACTGTCCTATCTGTGGGGCATCCTTTactcagaaaggtaacctcctccgccacattaaaCTG | 46F_47H_49P_50I_52G_53A_54S_55F_67L | 2.077927514 |
| 3369 | 3830 | TTCACCTGTCCCGTGTGTGGGGCAATGTTTactcagaaaggtaacctcctccgccacattaaaCTG | 46F_47T_49P_50V_52G_53A_54M_55F_67L | 2.077036592 |
| 3370 | 6677 | TTGATGTGTCCTATCTGCGGATTTGTGTGCactcagaaaggtaacctcctccgccacattaaaCTG | 46L_47M_49P_50I_52G_53F_54V_55C_67L | 2.07655185 |
| 3371 | 3677 | TTCACATGTGAAATCTGTGGGGCAGTTTTTactcagaaaggtaacctcctccgccacattaaaCTG | 46F_47T_49E_50I_52G_53A_54V_55F_67L | 2.076309517 |
| 3372 | 2596 | TTCATGTGTCCCATTTGTGGGGCAACCTTTactcagaaaggtaacctcctccgccacattaaaCTG | 46F_47M_49P_50I_52G_53A_54T_55F_67L | 2.075328264 |
| 3373 | 4423 | TTCTATTGTGAAGTTTGTGGGGCAGAGTTTactcagaaaggtaacctcctccgccacattaaaCTG | 46F_47Y_49E_50V_52G_53A_54E_55F_67L | 2.075162681 |
| 3374 | 6777 | CTCACGTGCCCAATTTGTGGCTTCGTTTGTactcagaaaggtaacctcctccgccacattaaaCTG | 46L_47T_49P_50I_52G_53F_54V_55C_67L | 2.075006901 |
| 3375 | 6719 | CTGCAGTGCCCGATCTGCGGGTTCTACTGCactcagaaaggtaacctcctccgccacattaaaCTG | 46L_47Q_49P_50I_52G_53F_54Y_55C_67L | 2.072644911 |
| 3376 | 2353 | TTCCTCTGTCCTATTTGTGGGGCACAGTTTactcagaaaggtaacctcctccgccacattaaaCTG | 46F_47L_49P_50I_52G_53A_54Q_55F_67L | 2.071626366 |
| 3377 | 1026 | TTCTTTTGTGAGATCTGTGGGGCACACTTTactcagaaaggtaacctcctccgccacattaaaCTG | 46F_47F_49E_50I_52G_53A_54H_55F_67L | 2.070549202 |
| 3378 | 4317 | TTCTGGTGTCCCGTCTGTGGGGCAGTTTTTactcagaaaggtaacctcctccgccacattaaaCTG | 46F_47W_49P_50V_52G_53A_54V_55F_67L | 2.068967815 |
| 3379 | 309 | TTCTGTTGTGAGATTTGTGGGGCATTATTTactcagaaaggtaacctoctocgccacattaaaCTG | 46F_47C_49E_50I_52G_53A_54L_55F_67L | 2.067374874 |
| 3380 | 2627 | TTCATGTGTCCTGTTTGTGGGGCAATTTTTactcagaaaggtaacctcctccgccacattaaaCTG | 46F_47M_49P_50V_52G_53A_54I_55F_67L | 2.066913347 |
| 3381 | 1429 | TTCGGATGTCCTGTCTGTGGGGCACTGTTTactcagaaaggtaacctcctccgccacattaaaCTG | 46F_47G_49P_50V_52G_53A_54L_55F_67L | 2.06527252 |
| 3382 | 4149 | TTCTGGTGTGAAATCTGTGGGGCACTGTTTactcagaaaggtaacctcctccgccacattaaaCTG | 46F_47W_49E_50I_52G_53A_54L_55F_67L | 2.064657921 |
| 3383 | 1261 | TTCGGCTGTGAAATCTGTGGGGCATGCTTTactcagaaaggtaacctcctccgccacattaaaCTG | 46F_47G_49E_50I_52G_53A_54C_55F_67L | 2.063952321 |
| 3384 | 6785 | TTGGTGTGCCCCATATGTGGGTTCGGGTGTactcagaaaggtaacctcctccgccacattaaaCTG | 46L_47V_49P_50I_52G_53F_54G_55C_67L | 2.063144907 |
| 3385 | 6707 | TTGCAGTGCCCAATTTGCGGTTTCATTTGTactcagaaaggtaacctcctccgccacattaaaCTG | 46L_47Q_49P_50I_52G_53F_54I_55C_67L | 2.061324264 |
| 3386 | 1893 | TTCATCTGTCCGCAGTGTGGGGCACAGTTTactcagaaaggtaacctcctccgccacattaaaCTG | 46F_47I_49P_50Q_52G_53A_54Q_55F_67L | 2.06131923 |
| 3387 | 1436 | TTCGGCTGTCCGGTGTGTGGGGCAACGTTTactcagaaaggtaacctcctccgccacattaaaCTG | 46F_47G_49P_50V_52G_53A_54T_55F_67L | 2.060635638 |
| 3388 | 4383 | TTCTACTGTGAAATCTGTGGGGCAGAGTTTactcagaaaggtaacctcctccgccacattaaaCTG | 46F_47Y_49E_50I_52G_53A_54E_55F_67L | 2.059437766 |
| 3389 | 6667 | CTGATGTGTCCTATCTGTGGGTTCATATGCactcagaaaggtaacctcctccgccacattaaaCTG | 46L_47M_49P_50I_52G_53F_54I_55C_67L | 2.05732325 |
| 3390 | 3700 | TTCACATGTGAAGTGTGTGGGGCAGCTTTTactcagaaaggtaacctcctccgccacattaaaCTG | 46F_47T_49E_50V_52G_53A_54A_55F_67L | 2.055753711 |
| 3391 | 376 | TTCTGTTGTAACATTTGTGGGGCAACATTTactcagaaaggtaacctcctccgccacattaaaCTG | 46F_47C_49N_50I_52G_53A_54T_55F_67L | 2.055502448 |
| 3392 | 429 | TTCTGTTGTCCTATTTGTGGGGCATTGTTTactcagaaaggtaacctcctccgccacattaaaCTG | 46F_47C_49P_50I_52G_53A_54L_55F_67L | 2.051874071 |
| 3393 | 1870 | TTCATCTGTCCCATTTGTGGGGCAATGTTTactcagaaaggtaacctcctccgccacattaaaCTG | 46F_47I_49P_50I_52G_53A_54M_55F_67L | 2.050229048 |
| 3394 | 6550 | CTTTTCTGCCCAATCTGCGGATTCATGTGTactcagaaaggtaacctcctccgccacattaaaCTG | 46L_47F_49P_50I_52G_53F_54M_55C_67L | 2.048928532 |
| 3395 | 780 | TTCGAGTGTGAGATCTGTGGGGCAGCGTTTactcagaaaggtaacctcctccgccacattaaaCTG | 46F_47E_49E_50I_52G_53A_54A_55F_67L | 2.048266703 |
| 3396 | 7686 | CTGTTTTGCGAAATCTGCGGATTTCATTGTactcagaaaggtaacctcctccgccacattaaaCTG | 46L_47F_49E_50I_52G_53F_54H_55C_67L | 2.04644351 |
| 3397 | 6460 | CTAGCTTGTCCAATCTGCGGTTTTGCATGTactcagaaaggtaacctcctccgccacattaaaCTG | 46L_47A_49P_50I_52G_53F_54A_55C_67L | 2.045868322 |
| 3398 | 942 | TTCGAATGTCCTGTGTGTGGGGCAGACTTTactcagaaaggtaacctcctccgccacattaaaCTG | 46F_47E_49P_50V_52G_53A_54D_55F_67L | 2.045267202 |
| 3399 | 4037 | TTCGTGTGTCCTATCTGTGGGGCAGTCTTTactcagaaaggtaacctcctccgccacattaaaCTG | 46F_47V_49P_50I_52G_53A_54V_55F_67L | 2.044398535 |
| 3400 | 4020 | TTCGTGTGTCCGATCTGTGGGGCAGCGTTTactcagaaaggtaacctcctccgccacattaaaCTG | 46F_47V_49P_50I_52G_53A_54A_55F_67L | 2.043471654 |
| 3401 | 6710 | CTGCAATGTCCAATCTGTGGCTTTATGTGCactcagaaaggtaacctcctccgccacattaaaCTG | 46L_47Q_49P_50I_52G_53F_54M_55C_67L | 2.04327747 |
| 3402 | 1787 | TTCATCTGTGAAGTCTGTGGGGCAATTTTTactcagaaaggtaacctcctccgccacattaaaCTG | 46F_47I_49E_50V_52G_53A_54I_55F_67L | 2.043162139 |
| 3403 | 1675 | TTCCATTGTCCAGTTTGTGGGGCATCATTTactcagaaaggtaacctcctccgccacattaaaCTG | 46F_47H_49P_50V_52G_53A_54S_55F_67L | 2.042089997 |
| 3404 | 6703 | CTGCAGTGCCCCATTTGCGGGTTCGAGTGTactcagaaaggtaacctcctccgccacattaaaCTG | 46L_47Q_49P_50I_52G_53F_54E_55C_67L | 2.041147853 |
| 3405 | 711 | TTCGATTGTCCAGTGTGTGGGGCAAACTTTactcagaaaggtaacctcctccgccacattaaaCTG | 46F_47D_49P_50V_52G_53A_54N_55F_67L | 2.040878975 |
| 3406 | 6793 | CTGGTCTGCCCTATCTGCGGGTTTCAGTGCactcagaaaggtaacctcctccgccacattaaaCTG | 46L_47V_49P_50I_52G_53F_54Q_55C_67L | 2.039478309 |
| 3407 | 7750 | TTGATCTGTGAGATCTGCGGGTTCATGTGTactcagaaaggtaacctcctccgccacattaaaCTG | 46L_47I_49E_50I_52G_53F_54M_55C_67L | 2.038918239 |
| 3408 | 4025 | TTCGTCTGTCCAATATGTGGGGCAGGCTTTactcagaaaggtaacctcctccgccacattaaaCTG | 46F_47V_49P_50I_52G_53A_54G_55F_67L | 2.038389802 |
| 3409 | 831 | TTCGAGTGTGAAGTGTGTGGGGCAAACTTTactcagaaaggtaacctcctccgccacattaaaCTG | 46F_47E_49E_50V_52G_53A_54N_55F_67L | 2.038113647 |
| 3410 | 1901 | TTCATCTGTCCAGTATGTGGGGCATGCTTTactcagaaaggtaacctcctccgccacattaaaCTG | 46F_47I_49P_50V_52G_53A_54C_55F_67L | 2.038023776 |
| 3411 | 3497 | TTCAGCTGTAACATTTGTGGGGCAGTGTTTactcagaaaggtaacctcctccgccacattaaaCTG | 46F_47S_49N_50I_52G_53A_54V_55F_67L | 2.03748762 |
| 3412 | 4535 | TTCTATTGTCCGCAGTGTGGGGCATCCTTTactcagaaaggtaacctcctccgccacattaaaCTG | 46F_47Y_49P_50Q_52G_53A_54S_55F_67L | 2.037134004 |
| 3413 | 1431 | TTCGGATGTCCAGTATGTGGGGCAAACTTTactcagaaaggtaacctcctccgccacattaaaCTG | 46F_47G_49P_50V_52G_53A_54N_55F_67L | 2.034305258 |
| 3414 | 1773 | TTCATTTGTGAGCAGTGTGGGGCACAGTTTactcagaaaggtaacctcctccgccacattaaaCTG | 46F_47I_49E_50Q_52G_53A_54Q_55F_67L | 2.033545256 |
| 3415 | 2172 | TTCCTGTGTGATATCTGTGGGGCACCATTTactcagaaaggtaacctcctccgccacattaaaCTG | 46F_47L_49D_50I_52G_53A_54P_55F_67L | 2.032819428 |
| 3416 | 2952 | TTCCAGTGTGAGATTTGTGGGGCACCATTTactcagaaaggtaacctcctccgccacattaaaCTG | 46F_47Q_49E_50I_52G_53A_54P_55F_67L | 2.031431287 |
| 3417 | 1637 | TTCCACTGTCCCATCTGTGGGGCAGTGTTTactcagaaaggtaacctcctccgccacattaaaCTG | 46F_47H_49P_50I_52G_53A_54V_55F_67L | 2.030897931 |
| 3418 | 3827 | TTCACCTGTCCCGTATGTGGGGCAATTTTTactcagaaaggtaacctcctccgccacattaaaCTG | 46F_47T_49P_50V_52G_53A_54I_55F_67L | 2.030735954 |
| 3419 | 1339 | TTCGGATGTAATATCTGTGGGGCATACTTTactcagaaaggtaacctcctccgccacattaaaCTG | 46F_47G_49N_50I_52G_53A_54Y_55F_67L | 2.02983724 |
| 3420 | 1791 | TTCATTTGTGAGGTGTGTGGGGCAAACTTTactcagaaaggtaacctcctccgccacattaaaCTG | 46F_47I_49E_50V_52G_53A_54N_55F_67L | 2.026457746 |
| 3421 | 1552 | TTCCACTGTGAGGTGTGTGGGGCACCTTTTactcagaaaggtaacctcctccgccacattaaaCTG | 46F_47H_49E_50V_52G_53A_54P_55F_67L | 2.026346599 |
| 3422 | 6545 | CTCTTCTGCCCAATCTGTGGCTTTGGATGTactcagaaaggtaacctcctccgccacattaaaCTG | 46L_47F_49P_50I_52G_53F_54G_55C_67L | 2.025100485 |
| 3423 | 2455 | TTCATGTGTGATGTTTGTGGGGCATCCTTTactcagaaaggtaacctcctccgccacattaaaCTG | 46F_47M_49D_50V_52G_53A_54S_55F_67L | 2.024808389 |
| 3424 | 7674 | CTCGAGTGCGAAATTTGTGGATTCCGGTGCactcagaaaggtaacctcctccgccacattaaaCTG | 46L_47E_49E_50I_52G_53F_54R_55C_67L | 2.023928564 |
| 3425 | 1317 | TTCGGATGTGAAGTCTGTGGGGCAGTGTTTactcagaaaggtaacctcctccgccacattaaaCTG | 46F_47G_49E_50V_52G_53A_54V_55F_67L | 2.023795073 |
| 3426 | 3782 | TTCACTTGTCCTATATGTGGGGCAGACTTTactcagaaaggtaacctcctccgccacattaaaCTG | 46F_47T_49P_50I_52G_53A_54D_55F_67L | 2.02104594 |
| 3427 | 2463 | TTCATGTGTGAAATCTGTGGGGCAGAGTTTactcagaaaggtaacctcctccgccacattaaaCTG | 46F_47M_49E_50I_52G_53A_54E_55F_67L | 2.02100806 |
| 3428 | 3679 | TTCACATGTGAAATCTGTGGGGCATACTTTactcagaaaggtaacctcctccgccacattaaaCTG | 46F_47T_49E_50I_52G_53A_54Y_55F_67L | 2.020237629 |
| 3429 | 2533 | TTCATGTGTAACATATGTGGGGCACAGTTTactcagaaaggtaacctcctccgccacattaaaCTG | 46F_47M_49N_50I_52G_53A_54Q_55F_67L | 2.019804625 |
| 3430 | 4332 | TTCTATTGTGATATATGTGGGGCACCGTTTactcagaaaggtaacctcctccgccacattaaaCTG | 46F_47Y_49D_50I_52G_53A_54P_55F_67L | 2.019170646 |
| 3431 | 3552 | TTCTCATGTCCCATCTGTGGGGCACCGTTTactcagaaaggtaacctcctccgccacattaaaCTG | 46F_47S_49P_50I_52G_53A_54P_55F_67L | 2.018757569 |
| 3432 | 4510 | TTCTACTGTCCCATTTGTGGGGCAATGTTTactcagaaaggtaacctcctccgccacattaaaCTG | 46F_47Y_49P_50I_52G_53A_54M_55F_67L | 2.018222602 |
| 3433 | 3826 | TTCACGTGTCCAGTGTGTGGGGCACACTTTactcagaaaggtaacctcctccgccacattaaaCTG | 46F_47T_49P_50V_52G_53A_54H_55F_67L | 2.018136588 |
| 3434 | 441 | TTCTGCTGTCCGCAGTGTGGGGCATGCTTTactcagaaaggtaacctcctccgccacattaaaCTG | 46F_47C_49P_50Q_52G_53A_54C_55F_67L | 2.015034727 |
| 3435 | 1871 | TTCATCTGTCCTATCTGTGGGGCAAACTTTactcagaaaggtaacctcctccgccacattaaaCTG | 46F_47I_49P_50I_52G_53A_54N_55F_67L | 2.013853029 |
| 3436 | 3195 | TTCAGGTGTGAAATCTGTGGGGCAAGCTTTactcagaaaggtaacctcctccgccacattaaaCTG | 46F_47R_49E_50I_52G_53A_54S_55F_67L | 2.012592548 |
| 3437 | 7681 | CTGTTTTGTGAAATTTGTGGATTTTGTTGTactcagaaaggtaacctcctccgccacattaaaCTG | 46L_47F_49E_50I_52G_53F_54C_55C_67L | 2.011187611 |
| 3438 | 2996 | TTCCAGTGTGAGGTCTGTGGGGCAACGTTTactcagaaaggtaacctcctccgccacattaaaCTG | 46F_47Q_49E_50V_52G_53A_54T_55F_67L | 2.010600788 |
| 3439 | 4521 | TTCTACTGTCCTCAGTGTGGGGCATGCTTTactcagaaaggtaacctcctccgccacattaaaCTG | 46F_47Y_49P_50Q_52G_53A_54C_55F_67L | 2.008118038 |
| 3440 | 4075 | TTCGTCTGTCCTGTGTGTGGGGCATCTTTTactcagaaaggtaacctcctccgccacattaaaCTG | 46F_47V_49P_50V_52G_53A_54S_55F_67L | 2.008004391 |
| 3441 | 2586 | TTCATGTGTCCAATCTGTGGGGCACATTTTactcagaaaggtaacctcctccgccacattaaaCTG | 46F_47M_49P_50I_52G_53A_54H_55F_67L | 2.007587584 |
| 3442 | 1545 | TTCCATTGTGAGGTCTGTGGGGCAGGCTTTactcagaaaggtaacctcctccgccacattaaaCTG | 46F_47H_49E_50V_52G_53A_54G_55F_67L | 2.006114488 |
| 3443 | 1070 | TTCTTTTGTGAAGTGTGTGGGGCAATGTTTactcagaaaggtaacctcctccgccacattaaaCTG | 46F_47F_49E_50V_52G_53A_54M_55F_67L | 2.004288295 |
| 3444 | 2525 | TTCATGTGTAACATTTGTGGGGCAGGTTTTactcagaaaggtaacctcctccgccacattaaaCTG | 46F_47M_49N_50I_52G_53A_54G_55F_67L | 2.003993794 |
| 3445 | 3790 | TTCACATGTCCAATCTGTGGGGCAATGTTTactcagaaaggtaacctcctccgccacattaaaCTG | 46F_47T_49P_50I_52G_53A_54M_55F_67L | 2.002748454 |
| 3446 | 4270 | TTCTGGTGTCCAATTTGTGGGGCAATGTTTactcagaaaggtaacctcctccgccacattaaaCTG | 46F_47W_49P_50I_52G_53A_54M_55F_67L | 2.002601403 |
| 3447 | 1853 | TTCATATGTAACGTGTGTGGGGCACAGTTTactcagaaaggtaacctcctccgccacattaaaCTG | 46F_47I_49N_50V_52G_53A_54Q_55F_67L | 2.001547899 |
| 3448 | 919 | TTCGAGTGTCCAATTTGTGGGGCATACTTTactcagaaaggtaacctcctccgccacattaaaCTG | 46F_47E_49P_50I_52G_53A_54Y_55F_67L | 2.001233794 |
| 3449 | 2560 | TTCATGTGTAACGTGTGTGGGGCAGCCTTTactcagaaaggtaacctcctccgccacattaaaCTG | 46F_47M_49N_50V_52G_53A_54A_55F_67L | 2.001149523 |
| 3450 | 1513 | TTCCATTGTGAGATCTGTGGGGCACAATTTactcagaaaggtaacctcctccgccacattaaaCTG | 46F_47H_49E_50I_52G_53A_54Q_55F_67L | 2.000452089 |

TABLE 5
B03 orthogonality; Estimated LogFC Coefficient calculated from the limma voom linear regression contrast of the positive vs negative read
counts for each individual mutant degron as defined relative to SEQ ID NO:2.

| | | | Annotation of amino acids according | |
|---|---|---|---|---|
| | | | to their position in SEQ ID NO: 2, | |
| | | | wherein the stated one letter | |
| | | | amino acid may be the original amino | |
| | | | acid found in SEQ ID NO: 2 or a | |
| | | DNA sequence encoding positions F146-L167 of SEQ ID NO: 2 absent any | substitution (first numerical digit | |
| SEQ ID | Degron- | substitutions defined according to the encoding sequence below and | dropped, i.e. position 146 in | |
| NO | ID | the variant amino acid sequence set out in column 4 of this Table | SEQ ID NO: 2 = 46 as stated below) | B03_logFC |
| 3451 | 4436 | TTCTACTGTGAGGTTTGTGGGGCAACGTTTactcagaaaggtaacctcctccgccacattaaaCTG | 46F_47Y_49E_50V_52G_53A_54T_55F_67L_ | 4.139533466 |
| 3452 | 4396 | TTCTACTGTGAGATCTGTGGGGCAACATTTactcagaaaggtaacctcctccgccacattaaaCTG | 46F_47Y_49E_50I_52G_53A_54T_55F_67L_ | 4.054136935 |
| 3453 | 3192 | TTCCGCTGTGAAATCTGTGGGGCACCCTTTactcagaaaggtaacctcctccgccacattaaaCTG | 46F_47R_49E_50I_52G_53A_54P_55F_67L_ | 3.983553562 |
| 3454 | 1912 | TTCATCTGTCCAGTCTGTGGGGCACCCTTTactcagaaaggtaacctcctccgccacattaaaCTG | 46F_47I_49P_50V_52G_53A_54P_55F_67L_ | 3.9370307 |
| 3455 | 4153 | TTCTGGTGTGAAATTTGTGGGGCACAGTTTactcagaaaggtaacctcctccgccacattaaaCTG | 46F_47W_49E_50I_52G_53A_54Q_55F_67L_ | 3.929867453 |
| 3456 | 4313 | TTCTGGTGTCCAGTGTGTGGGGCACAGTTTactcagaaaggtaacctcctccgccacattaaaCTG | 46F_47W_49P_50V_52G_53A_54Q_55F_67L_ | 3.919032576 |
| 3457 | 4186 | TTCTGGTGTGAGGTGTGTGGGGCACATTTTactcagaaaggtaacctcctccgccacattaaaCTG | 46F_47W_49E_50V_52G_53A_54H_55F_67L_ | 3.918622231 |
| 3458 | 4180 | TTCTGGTGTGAGGTCTGTGGGGCAGCCTTTactcagaaaggtaacctcctccgccacattaaaCTG | 46F_47W_49E_50V_52G_53A_54A_55F_67L_ | 3.904136833 |
| 3459 | 7974 | CTGTATTGTGAAATTTGCGGCTTTCGCTGCactcagaaaggtaacctcctccgccacattaaaCTG | 46L_47Y_49E_50I_52G_53F_54R_55C_67L_ | 3.901822687 |
| 3460 | 4131 | TTCTGGTGTGACGTTTGTGGGGCAAATTTTactcagaaaggtaacctcctccgccacattaaaCTG | 46F_47W_49D_50V_52G_53A_54N_55F_67L_ | 3.89035311 |
| 3461 | 4431 | TTCTATTGTGAGGTATGTGGGGCAAACTTTactcagaaaggtaacctcctccgccacattaaaCTG | 46F_47Y_49E_50V_52G_53A_54N_55F_67L_ | 3.876180684 |
| 3462 | 4435 | TTCTACTGTGAAGTTTGTGGGGCAAGCTTTactcagaaaggtaacctcctccgccacattaaaCTG | 46F_47Y_49E_50V_52G_53A_54S_55F_67L_ | 3.864746331 |
| 3463 | 4188 | TTCTGGTGTGAAGTGTGTGGGGCAAAATTTactcagaaaggtaacctcctccgccacattaaaCTG | 46F_47W_49E_50V_52G_53A_54K_55F_67L_ | 3.857082757 |
| 3464 | 4191 | TTCTGGTGTGAGGTTTGTGGGGCAAACTTTactcagaaaggtaacctcctccgccacattaaaCTG | 46F_47W_49E_50V_52G_53A_54N_55F_67L_ | 3.856394101 |
| 3465 | 4311 | TTCTGGTGTCCCGTCTGTGGGGCAAATTTTactcagaaaggtaacctcctccgccacattaaaCTG | 46F_47W_49P_50V_52G_53A_54N_55F_67L_ | 3.854865447 |
| 3466 | 4133 | TTCTGGTGTGACGTTTGTGGGGCACAGTTTactcagaaaggtaacctcctccgccacattaaaCTG | 46F_47W_49D_50V_52G_53A_54Q_55F_67L_ | 3.851299757 |
| 3467 | 4421 | TTCTACTGTGAGGTGTGTGGGGCATGCTTTactcagaaaggtaacctcctccgccacattaaaCTG | 46F_47Y_49E_50V_52G_53A_54C_55F_67L_ | 3.845730244 |
| 3468 | 4388 | TTCTACTGTGAGATCTGTGGGGCAAAATTTactcagaaaggtaacctcctccgccacattaaaCTG | 46F_47Y_49E_50I_52G_53A_54K_55F_67L_ | 3.83910481 |
| 3469 | 1021 | TTCTTTTGTGAAATCTGTGGGGCATGCTTTactcagaaaggtaacctcctccgccacattaaaCTG | 46F_47F_49E_50I_52G_53A_54C_55F_67L_ | 3.835346603 |
| 3470 | 4140 | TTCTGGTGTGAAATTTGTGGGGCAGCTTTTactcagaaaggtaacctcctccgccacattaaaCTG | 46F_47W_49E_50I_52G_53A_54A_55F_67L_ | 3.830580312 |
| 3471 | 4094 | TTCTGGTGTGATATCTGTGGGGCACGCTTTactcagaaaggtaacctcctccgccacattaaaCTG | 46F_47W_49D_50I_52G_53A_54R_55F_67L_ | 3.82392272 |
| 3472 | 4194 | TTCTGGTGTGAGGTTTGTGGGGCACGCTTTactcagaaaggtaacctcctccgccacattaaaCTG | 46F_47W_49E_50V_52G_53A_54R_55F_67L_ | 3.817395915 |
| 3473 | 4155 | TTCTGGTGTGAGATTTGTGGGGCATCTTTTactcagaaaggtaacctcctccgccacattaaaCTG | 46F_47W_49E_50I_52G_53A_54S_55F_67L_ | 3.809405654 |
| 3474 | 1036 | TTCTTTTGTGAAATTTGTGGGGCAACTTTTactcagaaaggtaacctcctccgccacattaaaCTG | 46F_47F_49E_50I_52G_53A_54T_55F_67L_ | 3.804185529 |
| 3475 | 4434 | TTCTACTGTGAGGTCTGTGGGGCAAGGTTTactcagaaaggtaacctcctccgccacattaaaCTG | 46F_47Y_49E_50V_52G_53A_54R_55F_67L_ | 3.796500546 |
| 3476 | 7956 | CTCTGGTGTGAAATTTGCGGATTCACCTGCactcagaaaggtaacctcctccgccacattaaaCTG | 46L_47W_49E_50I_52G_53F_54T_55C_67L_ | 3.794523031 |
| 3477 | 4141 | TTCTGGTGTGAGATCTGTGGGGCATGCTTTactcagaaaggtaacctcctccgccacattaaaCTG | 46F_47W_49E_50I_52G_53A_54C_55F_67L_ | 3.794515377 |
| 3478 | 4193 | TTCTGGTGTGAGGTGTGTGGGGCACAGTTTactcagaaaggtaacctcctccgccacattaaaCTG | 46F_47W_49E_50V_52G_53A_54Q_55F_67L_ | 3.792741296 |
| 3479 | 7955 | CTGTGGTGCGAGATCTGCGGCTTCAGCTGTactcagaaaggtaacctcctccgccacattaaaCTG | 46L_47W_49E_50I_52G_53F_54S_55C_67L_ | 3.789395559 |
| 3480 | 4306 | TTCTGGTGTCCAGTCTGTGGGGCACACTTTactcagaaaggtaacctcctccgccacattaaaCTG | 46F_47W_49P_50V_52G_53A_54H_55F_67L_ | 3.78832736 |
| 3481 | 4437 | TTCTACTGTGAGGTGTGTGGGGCAGTGTTTactcagaaaggtaacctcctccgccacattaaaCTG | 46F_47Y_49E_50V_52G_53A_54V_55F_67L_ | 3.786314244 |
| 3482 | 4146 | TTCTGGTGTGAGATCTGTGGGGCACACTTTactcagaaaggtaacctcctccgccacattaaaCTG | 46F_47W_49E_50I_52G_53A_54H_55F_67L_ | 3.783800348 |
| 3483 | 4151 | TTCTGGTGTGAGATATGTGGGGCAAATTTTactcagaaaggtaacctcctccgccacattaaaCTG | 46F_47W_49E_50I_52G_53A_54N_55F_67L_ | 3.781735181 |
| 3484 | 4181 | TTCTGGTGTGAAGTCTGTGGGGCATGCTTTactcagaaaggtaacctcctccgccacattaaaCTG | 46F_47W_49E_50V_52G_53A_54C_55F_67L_ | 3.768405042 |
| 3485 | 3312 | TTCCGCTGTCCCATCTGTGGGGCACCATTTactcagaaaggtaacctcctccgccacattaaaCTG | 46F_47R_49P_50I_52G_53A_54P_55F_67L_ | 3.762944086 |
| 3486 | 1037 | TTCTTTTGTGAGATCTGTGGGGCAGTGTTTactcagaaaggtaacctcctccgccacattaaaCTG | 46F_47F_49E_50I_52G_53A_54V_55F_67L_ | 3.757124313 |
| 3487 | 4148 | TTCTGGTGTGAAATCTGTGGGGCAAAGTTTactcagaaaggtaacctcctccgccacattaaaCTG | 46F_47W_49E_50I_52G_53A_54K_55F_67L_ | 3.754084895 |
| 3488 | 4196 | TTCTGGTGTGAAGTTTGTGGGGCAACATTTactcagaaaggtaacctcctccgccacattaaaCTG | 46F_47W_49E_50V_52G_53A_54T_55F_67L_ | 3.753415943 |
| 3489 | 4392 | TTCTATTGTGAAATTTGTGGGGCACCATTTactcagaaaggtaacctcctccgccacattaaaCTG | 46F_47Y_49E_50I_52G_53A_54P_55F_67L_ | 3.74280663 |
| 3490 | 1076 | TTCTTCTGTGAGGTGTGTGGGGCAACATTTactcagaaaggtaacctcctccgccacattaaaCTG | 46F_47F_49E_50V_52G_53A_54T_55F_67L_ | 3.736707475 |
| 3491 | 4156 | TTCTGGTGTGAAATCTGTGGGGCAACGTTTactcagaaaggtaacctcctccgccacattaaaCTG | 46F_47W_49E_50I_52G_53A_54T_55F_67L_ | 3.730714418 |
| 3492 | 1035 | TTCTTTTGTGAGATCTGTGGGGCAAGCTTTactcagaaaggtaacctcctccgccacattaaaCTG | 46F_47F_49E_50I_52G_53A_54S_55F_67L_ | 3.722320968 |
| 3493 | 6835 | TTGTACTGCCCTATATGTGGGTTCAGCTGCactcagaaaggtaacctcctccgccacattaaaCTG | 46L_47Y_49P_50I_52G_53F_54S_55C_67L_ | 3.721243845 |
| 3494 | 6816 | CTGTGGTGTCCCATCTGCGGCTTCACTTGTactcagaaaggtaacctcctccgccacattaaaCTG | 46L_47W_49P_50I_52G_53F_54T_55C_67L_ | 3.711850332 |
| 3495 | 7973 | CTCTATTGCGAGATCTGTGGATTTCAGTGTactcagaaaggtaacctcctccgccacattaaaCTG | 46L_47Y_49E_50I_52G_53F_54Q_55C_67L_ | 3.710773487 |
| 3496 | 1074 | TTCTTTTGTGAGGTATGTGGGGCAAGGTTTactcagaaaggtaacctcctccgccacattaaaCTG | 46F_47F_49E_50V_52G_53A_54R_55F_67L_ | 3.71014355 |
| 3497 | 4091 | TTCTGGTGTGACATTTGTGGGGCAAACTTTactcagaaaggtaacctcctccgccacattaaaCTG | 46F_47W_49D_50I_52G_53A_54N_55F_67L_ | 3.708153271 |
| 3498 | 6814 | CTTTGGTGCCCAATATGTGGCTTCCGGTGCactcagaaaggtaacctcctccgccacattaaaCTG | 46L_47W_49P_50I_52G_53F_54R_55C_67L_ | 3.704224768 |
| 3499 | 4420 | TTCTACTGTGAAGTCTGTGGGGCAGCTTTTactcagaaaggtaacctcctccgccacattaaaCTG | 46F_47Y_49E_50V_52G_53A_54A_55F_67L_ | 3.703736758 |
| 3500 | 4395 | TTCTATTGTGAGATCTGTGGGGCATCTTTTactcagaaaggtaacctcctccgccacattaaaCTG | 46F_47Y_49E_50I_52G_53A_54S_55F_67L_ | 3.69975359 |
| 3501 | 7975 | CTCTACTGCGAGATTTGCGGGTTTAGCTGCactcagaaaggtaacctcctccgccacattaaaCTG | 46L_47Y_49E_50I_52G_53F_54S_55C_67L_ | 3.698352403 |
| 3502 | 4088 | TTCTGGTGTGATATTTGTGGGGCAAAGTTTactcagaaaggtaacctcctccgccacattaaaCTG | 46F_47W_49D_50I_52G_53A_54K_55F_67L_ | 3.696440701 |
| 3503 | 4551 | TTCTACTGTCCAGTCTGTGGGGCAAATTTTactcagaaaggtaacctcctccgccacattaaaCTG | 46F_47Y_49P_50V_52G_53A_54N_55F_67L_ | 3.692115638 |
| 3504 | 4095 | TTCTGGTGTGATATCTGTGGGGCAAGTTTTactcagaaaggtaacctcctccgccacattaaaCTG | 46F_47W_49D_50I_52G_53A_54S_55F_67L_ | 3.69208461 |
| 3505 | 4273 | TTCTGGTGTCCAATTTGTGGGGCACAGTTTactcagaaaggtaacctcctccgccacattaaaCTG | 46F_47W_49P_50I_52G_53A_54Q_55F_67L_ | 3.688962571 |
| 3506 | 4300 | TTCTGGTGTCCAGTGTGTGGGGCAGCATTTactcagaaaggtaacctcctccgccacattaaaCTG | 46F_47W_49P_50V_52G_53A_54A_55F_67L_ | 3.687481272 |
| 3507 | 4032 | TTCGTGTGTCCTATCTGTGGGGCACCTTTTactcagaaaggtaacctcctccgccacattaaaCTG | 46F_47V_49P_50I_52G_53A_54P_55F_67L_ | 3.683354266 |
| 3508 | 4391 | TTCTACTGTGAAATTTGTGGGGCAAATTTTactcagaaaggtaacctcctccgccacattaaaCTG | 46F_47Y_49E_50I_52G_53A_54N_55F_67L_ | 3.6781915 |
| 3509 | 4093 | TTCTGGTGTGACATCTGTGGGGCACAGTTTactcagaaaggtaacctcctccgccacattaaaCTG | 46F_47W_49D_50I_52G_53A_54Q_55F_67L_ | 3.676264344 |
| 3510 | 7696 | CTGTTCTGTGAGATTTGTGGCTTCACGTGCactcagaaaggtaacctcctccgccacattaaaCTG | 46L_47F_49E_50I_52G_53F_54T_55C_67L_ | 3.670852959 |
| 3511 | 1033 | TTCTTCTGTGAGATCTGTGGGGCACAGTTTactcagaaaggtaacctcctccgccacattaaaCTG | 46F_47F_49E_50I_52G_53A_54Q_55F_67L_ | 3.669949758 |
| 3512 | 4080 | TTCTGGTGTGATATCTGTGGGGCAGCCTTTactcagaaaggtaacctcctccgccacattaaaCTG | 46F_47W_49D_50I_52G_53A_54A_55F_67L_ | 3.667894242 |
| 3513 | 1153 | TTCTTTTGTCCTATTTGTGGGGCACAGTTTactcagaaaggtaacctcctccgccacattaaaCTG | 46F_47F_49P_50I_52G_53A_54Q_55F_67L_ | 3.664183837 |
| 3514 | 4274 | TTCTGGTGTCCTATTTGTGGGGCACGATTTactcagaaaggtaacctcctccgccacattaaaCTG | 46F_47W_49P_50I_52G_53A_54R_55F_67L_ | 3.662966804 |
| 3515 | 1034 | TTCTTTTGTGAAATCTGTGGGGCAAGGTTTactcagaaaggtaacctcctccgccacattaaaCTG | 46F_47F_49E_50I_52G_53A_54R_55F_67L_ | 3.660685037 |
| 3516 | 1075 | TTCTTTTGTGAAGTCTGTGGGGCAAGCTTTactcagaaaggtaacctcctccgccacattaaaCTG | 46F_47F_49E_50V_52G_53A_54S_55F_67L_ | 3.659752688 |
| 3517 | 1151 | TTCTTCTGTCCCATTTGTGGGGCAAATTTTactcagaaaggtaacctcctccgccacattaaaCTG | 46F_47F_49P_50I_52G_53A_54N_55F_67L_ | 3.658010661 |
| 3518 | 4428 | TTCTATTGTGAAGTATGTGGGGCAAAGTTTactcagaaaggtaacctcctccgccacattaaaCTG | 46F_47Y_49E_50V_52G_53A_54K_55F_67L_ | 3.657786528 |
| 3519 | 1028 | TTCTTTTGTGAGATTTGTGGGGCAAAATTTactcagaaaggtaacctcctccgccacattaaaCTG | 46F_47F_49E_50I_52G_53A_54K_55F_67L_ | 3.654188269 |
| 3520 | 1872 | TTCATCTGTCCTATTTGTGGGGCACCTTTTactcagaaaggtaacctcctccgccacattaaaCTG | 46F_47I_49P_50I_52G_53A_54P_55F_67L_ | 3.65391607 |
| 3521 | 4081 | TTCTGGTGTGATATCTGTGGGGCATGCTTTactcagaaaggtaacctcctccgccacattaaaCTG | 46F_47W_49D_50I_52G_53A_54C_55F_67L_ | 3.65221659 |
| 3522 | 4553 | TTCTACTGTCCGGTGTGTGGGGCACAATTTactcagaaaggtaacctcctccgccacattaaaCTG | 46F_47Y_49P_50V_52G_53A_54Q_55F_67L_ | 3.651792934 |
| 3523 | 1020 | TTCTTCTGTGAGATCTGTGGGGCAGCTTTTactcagaaaggtaacctcctccgccacattaaaCTG | 46F_47F_49E_50I_52G_53A_54A_55F_67L_ | 3.644770249 |
| 3524 | 4271 | TTCTGGTGTCCCATCTGTGGGGCAAACTTTactcagaaaggtaacctcctccgccacattaaaCTG | 46F_47W_49P_50I_52G_53A_54N_55F_67L_ | 3.642701316 |
| 3525 | 7694 | CTTTTCTGTGAAATTTGCGGTTTCAGGTGTactcagaaaggtaacctcctccgccacattaaaCTG | 46L_47F_49E_50I_52G_53F_54R_55C_67L_ | 3.641248924 |
| 3526 | 4275 | TTCTGGTGTCCGATCTGTGGGGCAAGTTTTactcagaaaggtaacctcctccgccacattaaaCTG | 46F_47W_49P_50I_52G_53A_54S_55F_67L_ | 3.636522534 |
| 3527 | 4501 | TTCTATTGTCCTATCTGTGGGGCATGCTTTactcagaaaggtaacctcctccgccacattaaaCTG | 46F_47Y_49P_50I_52G_53A_54C_55F_67L_ | 3.633250903 |
| 3528 | 6836 | CTCTATTGTCCCATTTGTGGGTTCACTTGCactcagaaaggtaacctcctccgccacattaaaCTG | 46L_47Y_49P_50I_52G_53F_54T_55C_67L_ | 3.628464163 |
| 3529 | 1031 | TTCTTCTGTGAGATTTGTGGGGCAAATTTTactcagaaaggtaacctcctccgccacattaaaCTG | 46F_47F_49E_50I_52G_53A_54N_55F_67L_ | 3.628445789 |
| 3530 | 4150 | TTCTGGTGTGAGATATGTGGGGCAATGTTTactcagaaaggtaacctcctccgccacattaaaCTG | 46F_47W_49E_50I_52G_53A_54M_55F_67L_ | 3.628174332 |
| 3531 | 6795 | CTGGTGTGCCCCATCTGCGGCTTCTCTTGTactcagaaaggtaacctcctccgccacattaaaCTG | 46L_47V_49P_50I_52G_53F_545_55C_67L_ | 3.627893656 |
| 3532 | 4426 | TTCTACTGTGAGGTGTGTGGGGCACATTTTactcagaaaggtaacctcctccgccacattaaaCTG | 46F_47Y_49E_50V_52G_53A_54H_55F_67L_ | 3.622333352 |
| 3533 | 4211 | TTCTGGTGTAATATCTGTGGGGCAAACTTTactcagaaaggtaacctcctccgccacattaaaCTG | 46F_47W_49N_50I_52G_53A_54N_55F_67L_ | 3.61859388 |
| 3534 | 3912 | TTCGTGTGTGAAATCTGTGGGGCACCCTTTactcagaaaggtaacctcctccgccacattaaaCTG | 46F_47V_49E_50I_52G_53A_54P_55F_67L_ | 3.616486894 |
| 3535 | 3352 | TTCCGCTGTCCCGTTTGTGGGGCACCATTTactcagaaaggtaacctcctccgccacattaaaCTG | 46F_47R_49P_50V_52G_53A_54P_55F_67L_ | 3.61550439 |
| 3536 | 3952 | TTCGTGTGTGAGGTGTGTGGGGCACCTTTTactcagaaaggtaacctcctccgccacattaaaCTG | 46F_47V_49E_50V_52G_53A_54P_55F_67L_ | 3.614646322 |
| 3537 | 2952 | TTCCAGTGTGAGATTTGTGGGGCACCATTTactcagaaaggtaacctcctccgccacattaaaCTG | 46F_47Q_49E_50I_52G_53A_54P_55F_67L_ | 3.614565023 |
| 3538 | 1156 | TTCTTTTGTCCCATATGTGGGGCAACGTTTactcagaaaggtaacctcctccgccacattaaaCTG | 46F_47F_49P_50I_52G_53A_54T_55F_67L_ | 3.613031505 |
| 3539 | 1027 | TTCTTCTGTGAGATCTGTGGGGCAATTTTTactcagaaaggtaacctcctccgccacattaaaCTG | 46F_47F_49E_50I_52G_53A_54I_55F_67L_ | 3.607463204 |
| 3540 | 4381 | TTCTATTGTGAGATATGTGGGGCATGCTTTactcagaaaggtaacctcctccgccacattaaaCTG | 46F_47Y_49E_50I_52G_53A_54C_55F_67L_ | 3.604684908 |
| 3541 | 3072 | TTCCAATGTCCTATTTGTGGGGCACCATTTactcagaaaggtaacctcctccgccacattaaaCTG | 46F_47Q_49P_50I_52G_53A_54P_55F_67L_ | 3.604056375 |
| 3542 | 1073 | TTCTTCTGTGAGGTCTGTGGGGCACAGTTTactcagaaaggtaacctcctccgccacattaaaCTG | 46F_47F_49E_50V_52G_53A_54Q_55F_67L_ | 3.602116106 |
| 3543 | 4397 | TTCTACTGTGAGATATGTGGGGCAGTATTTactcagaaaggtaacctcctccgccacattaaaCTG | 46F_47Y_49E_50I_52G_53A_54V_55F_67L_ | 3.600440326 |
| 3544 | 3232 | TTCAGGTGTGAGGTTTGTGGGGCACCGTTTactcagaaaggtaacctcctccgccacattaaaCTG | 46F_47R_49E_50V_52G_53A_54P_55F_67L_ | 3.598529815 |
| 3545 | 4134 | TTCTGGTGTGACGTGTGTGGGGCACGCTTTactcagaaaggtaacctcctccgccacattaaaCTG | 46F_47W_49D_50V_52G_53A_54R_55F_67L_ | 3.597308708 |
| 3546 | 4213 | TTCTGGTGTAACATCTGTGGGGCACAGTTTactcagaaaggtaacctcctccgccacattaaaCTG | 46F_47W_49N_50I_52G_53A_54Q_55F_67L_ | 3.593206735 |
| 3547 | 4380 | TTCTATTGTGAAATCTGTGGGGCAGCCTTTactcagaaaggtaacctcctccgccacattaaaCTG | 46F_47Y_49E_50I_52G_53A_54A_55F_67L_ | 3.592086017 |
| 3548 | 2632 | TTCATGTGTCCAGTTTGTGGGGCACCCTTTactcagaaaggtaacctcctccgccacattaaaCTG | 46F_47M_49P_50V_52G_53A_54P_55F_67L_ | 3.588305979 |
| 3549 | 4433 | TTCTATTGTGAGGTTTGTGGGGCACAATTTactcagaaaggtaacctcctccgccacattaaaCTG | 46F_47Y_49E_50V_52G_53A_54Q_55F_67L_ | 3.572891941 |
| 3550 | 4516 | TTCTATTGTCCAATCTGTGGGGCAACATTTactcagaaaggtaacctcctccgccacattaaaCTG | 46F_47Y_49P_50I_52G_53A_54T_55F_67L_ | 3.572604526 |
| 3551 | 6815 | CTGTGGTGCCCGATCTGTGGATTTAGCTGCactcagaaaggtaacctcctccgccacattaaaCTG | 46L_47W_49P_50I_52G_53F_54S_55C_67L_ | 3.566798086 |
| 3552 | 4276 | TTCTGGTGTCCCATCTGTGGGGCAACCTTTactcagaaaggtaacctcctccgccacattaaaCTG | 46F_47W_49P_50I_52G_53A_54T_55F_67L_ | 3.565924832 |
| 3553 | 4332 | TTCTATTGTGATATATGTGGGGCACCGTTTactcagaaaggtaacctcctccgccacattaaaCTG | 46F_47Y_49D_50I_52G_53A_54P_55F_67L_ | 3.564515976 |
| 3554 | 4513 | TTCTACTGTCCTATCTGTGGGGCACAGTTTactcagaaaggtaacctcctccgccacattaaaCTG | 46F_47Y_49P_50I_52G_53A_54Q_55F_67L_ | 3.563023809 |
| 3555 | 6821 | TTGTATTGTCCCATTTGTGGCTTTTGTTGTactcagaaaggtaacctcctccgccacattaaaCTG | 46L_47Y_49P_50I_52G_53F_54C_55C_67L_ | 3.558939165 |
| 3556 | 4506 | TTCTACTGTCCGATCTGTGGGGCACATTTTactcagaaaggtaacctcctccgccacattaaaCTG | 46F_47Y_49P_50I_52G_53A_54H_55F_67L_ | 3.557811492 |
| 3557 | 6833 | CTCTACTGCCCTATTTGCGGGTTCCAGTGCactcagaaaggtaacctcctccgccacattaaaCTG | 46L_47Y_49P_50I_52G_53F_54Q_55C_67L_ | 3.557058564 |
| 3558 | 6555 | TTGTTTTGTCCTATATGCGGGTTTTCATGCactcagaaaggtaacctcctccgccacattaaaCTG | 46L_47F_49P_50I_52G_53F_54S_55C_67L_ | 3.556350348 |
| 3559 | 4333 | TTCTACTGTGACATTTGTGGGGCACAGTTTactcagaaaggtaacctcctccgccacattaaaCTG | 46F_47Y_49D_50I_52G_53A_54Q_55F_67L_ | 3.554100403 |
| 3560 | 2512 | TTCATGTGTGAAGTCTGTGGGGCACCCTTTactcagaaaggtaacctcctccgccacattaaaCTG | 46F_47M_49E_50V_52G_53A_54P_55F_67L_ | 3.548786012 |
| 3561 | 4147 | TTCTGGTGTGAGATTTGTGGGGCAATATTTactcagaaaggtaacctcctccgccacattaaaCTG | 46F_47W_49E_50I_52G_53A_54I_55F_67L_ | 3.548092781 |
| 3562 | 4393 | TTCTACTGTGAGATCTGTGGGGCACAATTTactcagaaaggtaacctcctccgccacattaaaCTG | 46F_47Y_49E_50I_52G_53A_54Q_55F_67L_ | 3.547771999 |
| 3563 | 6556 | CTGTTTTGTCCCATCTGCGGGTTCACATGTactcagaaaggtaacctcctccgccacattaaaCTG | 46L_47F_49P_50I_52G_53F_54T_55C_67L_ | 3.544994526 |
| 3564 | 4195 | TTCTGGTGTGAGGTGTGTGGGGCATCCTTTactcagaaaggtaacctcctccgccacattaaaCTG | 46F_47W_49E_50V_52G_53A_54S_55F_67L_ | 3.544736485 |
| 3565 | 4215 | TTCTGGTGTAATATATGTGGGGCAAGCTTTactcagaaaggtaacctcctccgccacattaaaCTG | 46F_47W_49N_50I_52G_53A_54S_55F_67L_ | 3.542992565 |
| 3566 | 4452 | TTCTACTGTAATATTTGTGGGGCACCATTTactcagaaaggtaacctcctccgccacattaaaCTG | 46F_47Y_49N_50I_52G_53A_54P_55F_67L_ | 3.540813699 |
| 3567 | 1196 | TTCTTCTGTCCTGTGTGTGGGGCAACTTTTactcagaaaggtaacctcctccgccacattaaaCTG | 46F_47F_49P_50V_52G_53A_54T_55F_67L_ | 3.537791006 |
| 3568 | 6806 | CTGTGGTGTCCTATCTGCGGGTTCCACTGTactcagaaaggtaacctcctccgccacattaaaCTG | 46L_47W_49P_50I_52G_53F_54H_55C_67L_ | 3.537472073 |
| 3569 | 4293 | TTCTGGTGTCCTCAGTGTGGGGCACAGTTTactcagaaaggtaacctcctccgccacattaaaCTG | 46F_47W_49P_50Q_52G_53A_54Q_55F_67L_ | 3.537236662 |
| 3570 | 6732 | CTGAGATGTCCTATTTGTGGCTTTCCCTGCactcagaaaggtaacctcctccgccacattaaaCTG | 46L_47R_49P_50I_52G_53F_54P_55C_67L_ | 3.535595176 |
| 3571 | 3132 | TTCAGGTGTGATATCTGTGGGGCACCCTTTactcagaaaggtaacctcctccgccacattaaaCTG | 46F_47R_49D_50I_52G_53A_54P_55F_67L_ | 3.531210992 |
| 3572 | 1752 | TTCATCTGTGAGATTTGTGGGGCACCTTTTactcagaaaggtaacctcctccgccacattaaaCTG | 46F_47I_49E_50I_52G_53A_54P_55F_67L_ | 3.530283257 |
| 3573 | 4387 | TTCTACTGTGAAATTTGTGGGGCAATCTTTactcagaaaggtaacctcctccgccacattaaaCTG | 46F_47Y_49E_50I_52G_53A_54I_55F_67L_ | 3.52720017 |
| 3574 | 4251 | TTCTGGTGTAATGTATGTGGGGCAAATTTTactcagaaaggtaacctcctccgccacattaaaCTG | 46F_47W_49N_50V_52G_53A_54N_55F_67L_ | 3.527029014 |
| 3575 | 6540 | CTGTTCTGCCCGATCTGCGGTTTCGCCTGCactcagaaaggtaacctcctccgccacattaaaCTG | 46L_47F_49P_50I_52G_53F_54A_55C_67L_ | 3.521362543 |
| 3576 | 6813 | CTGTGGTGTCCGATCTGCGGCTTTCAATGCactcagaaaggtaacctcctccgccacattaaaCTG | 46L_47W_49P_50I_52G_53F_54Q_55C_67L_ | 3.516491726 |
| 3577 | 4266 | TTCTGGTGTCCGATCTGTGGGGCACATTTTactcagaaaggtaacctcctccgccacattaaaCTG | 46F_47W_49P_50I_52G_53A_54H_55F_67L_ | 3.514274993 |
| 3578 | 1061 | TTCTTCTGTGAGGTGTGTGGGGCATGCTTTactcagaaaggtaacctcctccgccacattaaaCTG | 46F_47F_49E_50V_52G_53A_54C_55F_67L_ | 3.513580881 |
| 3579 | 1191 | TTCTTCTGTCCTGTCTGTGGGGCAAACTTTactcagaaaggtaacctcctccgccacattaaaCTG | 46F_47F_49P_50V_52G_53A_54N_55F_67L_ | 3.511010664 |
| 3580 | 7695 | CTTTTCTGCGAAATTTGTGGCTTCTCCTGCactcagaaaggtaacctcctccgccacattaaaCTG | 46L_47F_49E_50I_52G_53F_54S_55C_67L_ | 3.510921618 |
| 3581 | 4314 | TTCTGGTGTCCTGTGTGTGGGGCAAGATTTactcagaaaggtaacctcctccgccacattaaaCTG | 46F_47W_49P_50V_52G_53A_54R_55F_67L_ | 3.510048917 |
| 3582 | 4154 | TTCTGGTGTGAAATTTGTGGGGCACGTTTTactcagaaaggtaacctcctccgccacattaaaCTG | 46F_47W_49E_50I_52G_53A_54R_55F_67L_ | 3.508175948 |
| 3583 | 7976 | CTCTATTGCGAAATATGCGGTTTCACATGCactcagaaaggtaacctcctccgccacattaaaCTG | 46L_47Y_49E_50I_52G_53F_54T_55C_67L_ | 3.508140951 |
| 3584 | 4157 | TTCTGGTGTGAAATTTGTGGGGCAGTATTTactcagaaaggtaacctcctccgccacattaaaCTG | 46F_47W_49E_50I_52G_53A_54V_55F_67L_ | 3.50660745 |
| 3585 | 7960 | CTGTACTGTGAGATTTGCGGATTCGCTTGTactcagaaaggtaacctcctccgccacattaaaCTG | 46L_47Y_49E_50I_52G_53F_54A_55C_67L_ | 3.504435974 |
| 3586 | 7954 | CTTTGGTGTGAAATCTGCGGATTCAGATGTactcagaaaggtaacctcctccgccacattaaaCTG | 46L_47W_49E_50I_52G_53F_54R_55C_67L_ | 3.499228079 |
| 3587 | 6811 | CTGTGGTGTCCCATTTGCGGGTTCAATTGTactcagaaaggtaacctcctccgccacattaaaCTG | 46L_47W_49P_50I_52G_53F_54N_55C_67L_ | 3.497634953 |
| 3588 | 7953 | CTGTGGTGTGAGATTTGTGGCTTCCAGTGCactcagaaaggtaacctcctccgccacattaaaCTG | 46L_47W_49E_50I_52G_53F_54Q_55C_67L_ | 3.49273907 |
| 3589 | 6801 | CTCTGGTGTCCCATTTGTGGCTTTTGCTGCactcagaaaggtaacctcctccgccacattaaaCTG | 46L_47W_49P_50I_52G_53F_54C_55C_67L_ | 3.492478824 |
| 3590 | 6837 | CTATACTGCCCGATATGCGGGTTTGTCTGTactcagaaaggtaacctcctccgccacattaaaCTG | 46L_47Y_49P_50I_52G_53F_54V_55C_67L_ | 3.49155325 |
| 3591 | 2992 | TTCCAATGTGAGGTCTGTGGGGCACCATTTactcagaaaggtaacctcctccgccacattaaaCTG | 46F_47Q_49E_50V_52G_53A_54P_55F_67L_ | 3.489784831 |
| 3592 | 4386 | TTCTACTGTGAGATTTGTGGGGCACACTTTactcagaaaggtaacctcctccgccacattaaaCTG | 46F_47Y_49E_50I_52G_53A_54H_55F_67L_ | 3.489029272 |
| 3593 | 2592 | TTCATGTGTCCCATATGTGGGGCACCCTTTactcagaaaggtaacctcctccgccacattaaaCTG | 46F_47M_49P_50I_52G_53A_54P_55F_67L_ | 3.4885788 |
| 3594 | 6826 | CTCTACTGTCCCATCTGTGGCTTCCACTGCactcagaaaggtaacctcctccgccacattaaaCTG | 46L_47Y_49P_50I_52G_53F_54H_55C_67L_ | 3.488474002 |
| 3595 | 6595 | TTACATTGCCCAATATGTGGCTTCTCCTGTactcagaaaggtaacctcctccgccacattaaaCTG | 46L_47H_49P_50I_52G_53F_54S_55C_67L_ | 3.487759973 |
| 3596 | 3252 | TTCAGGTGTAACATTTGTGGGGCACCGTTTactcagaaaggtaacctcctccgccacattaaaCTG | 46F_47R_49N_50I_52G_53A_54P_55F_67L_ | 3.486969987 |
| 3597 | 4546 | TTCTATTGTCCTGTGTGTGGGGCACACTTTactcagaaaggtaacctcctccgccacattaaaCTG | 46F_47Y_49P_50V_52G_53A_54H_55F_67L_ | 3.484969582 |
| 3598 | 7946 | CTGTGGTGCGAAATTTGCGGGTTTCATTGCactcagaaaggtaacctcctccgccacattaaaCTG | 46L_47W_49E_50I_52G_53F_54H_55C_67L_ | 3.48415936 |
| 3599 | 1792 | TTCATCTGTGAGGTGTGTGGGGCACCATTTactcagaaaggtaacctcctccgccacattaaaCTG | 46F_47I_49E_50V_52G_53A_54P_55F_67L_ | 3.483822867 |
| 3600 | 4135 | TTCTGGTGTGACGTCTGTGGGGCATCTTTTactcagaaaggtaacctcctccgccacattaaaCTG | 46F_47W_49D_50V_52G_53A_54S_55F_67L_ | 3.481719735 |
| 3601 | 4315 | TTCTGGTGTCCCGTATGTGGGGCATCTTTTactcagaaaggtaacctcctccgccacattaaaCTG | 46F_47W_49P_50V_52G_53A_54S_55F_67L_ | 3.475196354 |
| 3602 | 1193 | TTCTTCTGTCCAGTGTGTGGGGCACAGTTTactcagaaaggtaacctcctccgccacattaaaCTG | 46F_47F_49P_50V_52G_53A_54Q_55F_67L_ | 3.47224171 |
| 3603 | 4121 | TTCTGGTGTGATGTATGTGGGGCATGCTTTactcagaaaggtaacctcctccgccacattaaaCTG | 46F_47W_49D_50V_52G_53A_54C_55F_67L_ | 3.4711717 |
| 3604 | 1692 | TTCATATGTGATATCTGTGGGGCACCATTTactcagaaaggtaacctcctccgccacattaaaCTG | 46F_47I_49D_50I_52G_53A_54P_55F_67L_ | 3.464405983 |
| 3605 | 4511 | TTCTATTGTCCCATTTGTGGGGCAAACTTTactcagaaaggtaacctcctccgccacattaaaCTG | 46F_47Y_49P_50I_52G_53A_54N_55F_67L_ | 3.463110699 |
| 3606 | 6541 | CTGTTCTGCCCCATATGTGGGTTCTGTTGTactcagaaaggtaacctcctccgccacattaaaCTG | 46L_47F_49P_50I_52G_53F_54C_55C_67L_ | 3.462705365 |
| 3607 | 1512 | TTCCATTGTGAGATCTGTGGGGCACCATTTactcagaaaggtaacctcctccgccacattaaaCTG | 46F_47H_49E_50I_52G_53A_54P_55F_67L_ | 3.462033313 |
| 3608 | 4190 | TTCTGGTGTGAAGTGTGTGGGGCAATGTTTactcagaaaggtaacctcctccgccacattaaaCTG | 46F_47W_49E_50V_52G_53A_54M_55F_67L_ | 3.457599478 |
| 3609 | 4427 | TTCTATTGTGAAGTTTGTGGGGCAATCTTTactcagaaaggtaacctcctccgccacattaaaCTG | 46F_47Y_49E_50V_52G_53A_54I_55F_67L_ | 3.453344445 |
| 3610 | 4432 | TTCTACTGTGAAGTATGTGGGGCACCATTTactcagaaaggtaacctcctccgccacattaaaCTG | 46F_47Y_49E_50V_52G_53A_54P_55F_67L_ | 3.453324012 |
| 3611 | 7697 | CTGTTCTGCGAGATTTGCGGATTTGTCTGCactcagaaaggtaacctcctccgccacattaaaCTG | 46L_47F_49E_50I_52G_53F_54V_55C_67L_ | 3.452839356 |
| 3612 | 4316 | TTCTGGTGTCCAGTTTGTGGGGCAACCTTTactcagaaaggtaacctcctccgccacattaaaCTG | 46F_47W_49P_50V_52G_53A_54T_55F_67L_ | 3.450194874 |
| 3613 | 1071 | TTCTTCTGTGAGGTCTGTGGGGCAAACTTTactcagaaaggtaacctcctccgccacattaaaCTG | 46F_47F_49E_50V_52G_53A_54N_55F_67L_ | 3.445032579 |
| 3614 | 4301 | TTCTGGTGTCCGGTTTGTGGGGCATGCTTTactcagaaaggtaacctcctccgccacattaaaCTG | 46F_47W_49P_50V_52G_53A_54C_55F_67L_ | 3.444070588 |
| 3615 | 3792 | TTCACATGTCCCATCTGTGGGGCACCGTTTactcagaaaggtaacctcctccgccacattaaaCTG | 46F_47T_49P_50I_52G_53A_54P_55F_67L_ | 3.443801235 |
| 3616 | 1077 | TTCTTCTGTGAGGTGTGTGGGGCAGTGTTTactcagaaaggtaacctcctccgccacattaaaCTG | 46F_47F_49E_50V_52G_53A_54V_55F_67L_ | 3.441145769 |
| 3617 | 1152 | TTCTTCTGTCCAATTTGTGGGGCACCATTTactcagaaaggtaacctcctccgccacattaaaCTG | 46F_47F_49P_50I_52G_53A_54P_55F_67L_ | 3.440696824 |
| 3618 | 1141 | TTCTTTTGTCCGATTTGTGGGGCATGCTTTactcagaaaggtaacctcctccgccacattaaaCTG | 46F_47F_49P_50I_52G_53A_54C_55F_67L_ | 3.436439161 |
| 3619 | 2472 | TTCATGTGTGAGATTTGTGGGGCACCATTTactcagaaaggtaacctcctccgccacattaaaCTG | 46F_47M_49E_50I_52G_53A_54P_55F_67L_ | 3.432403607 |
| 3620 | 4515 | TTCTACTGTCCGATCTGTGGGGCAAGTTTTactcagaaaggtaacctcctccgccacattaaaCTG | 46F_47Y_49P_50I_52G_53A_54S_55F_67L_ | 3.432207773 |
| 3621 | 4268 | TTCTGGTGTCCCATCTGTGGGGCAAAGTTTactcagaaaggtaacctcctccgccacattaaaCTG | 46F_47W_49P_50I_52G_53A_54K_55F_67L_ | 3.431046237 |
| 3622 | 1812 | TTCATCTGTAATATTTGTGGGGCACCCTTTactcagaaaggtaacctcctccgccacattaaaCTG | 46F_47I_49N_50I_52G_53A_54P_55F_67L_ | 3.429472806 |
| 3623 | 4430 | TTCTATTGTGAAGTTTGTGGGGCAATGTTTactcagaaaggtaacctcctccgccacattaaaCTG | 46F_47Y_49E_50V_52G_53A_54M_55F_67L_ | 3.427732165 |
| 3624 | 4197 | TTCTGGTGTGAGGTGTGTGGGGCAGTCTTTactcagaaaggtaacctcctccgccacattaaaCTG | 46F_47W_49E_50V_52G_53A_54V_55F_67L_ | 3.42641069 |
| 3625 | 4331 | TTCTATTGTGATATTTGTGGGGCAAACTTTactcagaaaggtaacctcctccgccacattaaaCTG | 46F_47Y_49D_50I_52G_53A_54N_55F_67L_ | 3.424068329 |
| 3626 | 4390 | TTCTACTGTGAGATATGTGGGGCAATGTTTactcagaaaggtaacctcctccgccacattaaaCTG | 46F_47Y_49E_50I_52G_53A_54M_55F_67L_ | 3.419142804 |
| 3627 | 7966 | CTGTACTGCGAGATCTGTGGCTTCCATTGTactcagaaaggtaacctcctccgccacattaaaCTG | 46L_47Y_49E_50I_52G_53F_54H_55C_67L_ | 3.417751061 |
| 3628 | 1068 | TTCTTTTGTGAGGTGTGTGGGGCAAAATTTactcagaaaggtaacctcctccgccacattaaaCTG | 46F_47F_49E_50V_52G_53A_54K_55F_67L_ | 3.417509351 |
| 3629 | 4152 | TTCTGGTGTGAAATTTGTGGGGCACCATTTactcagaaaggtaacctcctccgccacattaaaCTG | 46F_47W_49E_50I_52G_53A_54P_55F_67L_ | 3.417395796 |
| 3630 | 4321 | TTCTACTGTGACATTTGTGGGGCATGTTTTactcagaaaggtaacctcctccgccacattaaaCTG | 46F_47Y_49D_50I_52G_53A_54C_55F_67L_ | 3.415300059 |
| 3631 | 7693 | TTATTCTGTGAGATCTGCGGTTTTCAGTGTactcagaaaggtaacctcctccgccacattaaaCTG | 46L_47F_49E_50I_52G_53F_54Q_55C_67L_ | 3.410768732 |
| 3632 | 4086 | TTCTGGTGTGACATATGTGGGGCACACTTTactcagaaaggtaacctcctccgccacattaaaCTG | 46F_47W_49D_50I_52G_53A_54H_55F_67L_ | 3.406659373 |
| 3633 | 4541 | TTCTACTGTCCGGTTTGTGGGGCATGCTTTactcagaaaggtaacctcctccgccacattaaaCTG | 46F_47Y_49P_50V_52G_53A_54C_55F_67L_ | 3.401414382 |
| 3634 | 6834 | CTTTATTGTCCCATCTGTGGATTTCGGTGTactcagaaaggtaacctcctccgccacattaaaCTG | 46L_47Y_49P_50I_52G_53F_54R_55C_67L_ | 3.395413347 |
| 3635 | 1060 | TTCTTCTGTGAGGTTTGTGGGGCAGCTTTTactcagaaaggtaacctcctccgccacattaaaCTG | 46F_47F_49E_50V_52G_53A_54A_55F_67L_ | 3.388874973 |
| 3636 | 7681 | CTGTTTTGTGAAATTTGTGGATTTTGTTGTactcagaaaggtaacctcctccgccacattaaaCTG | 46L_47F_49E_50I_52G_53F_54C_55C_67L_ | 3.386301019 |
| 3637 | 1632 | TTCCACTGTCCAATTTGTGGGGCACCTTTTactcagaaaggtaacctcctccgccacattaaaCTG | 46F_47H_49P_50I_52G_53A_54P_55F_67L_ | 3.386219982 |
| 3638 | 4556 | TTCTATTGTCCGGTGTGTGGGGCAACTTTTactcagaaaggtaacctcctccgccacattaaaCTG | 46F_47Y_49P_50V_52G_53A_54T_55F_67L_ | 3.380780989 |
| 3639 | 4389 | TTCTACTGTGAAATCTGTGGGGCACTGTTTactcagaaaggtaacctcctccgccacattaaaCTG | 46F_47Y_49E_50I_52G_53A_54L_55F_67L_ | 3.378916231 |
| 3640 | 4326 | TTCTATTGTGATATTTGTGGGGCACATTTTactcagaaaggtaacctcctccgccacattaaaCTG | 46F_47Y_49D_50I_52G_53A_54H_55F_67L_ | 3.376087986 |
| 3641 | 4120 | TTCTGGTGTGACGTCTGTGGGGCAGCATTTactcagaaaggtaacctcctccgccacattaaaCTG | 46F_47W_49D_50V_52G_53A_54A_55F_67L_ | 3.372501647 |
| 3642 | 6810 | CTGTGGTGCCCCATTTGTGGCTTCATGTGTactcagaaaggtaacctcctccgccacattaaaCTG | 46L_47W_49P_50I_52G_53F_54M_55C_67L_ | 3.371345599 |
| 3643 | 6775 | CTCACCTGCCCAATCTGCGGGTTCAGCTGTactcagaaaggtaacctcctccgccacattaaaCTG | 46L_47T_49P_50I_52G_53F_54S_55C_67L_ | 3.37067871 |
| 3644 | 4394 | TTCTACTGTGAAATCTGTGGGGCAAGATTTactcagaaaggtaacctcctccgccacattaaaCTG | 46F_47Y_49E_50I_52G_53A_54R_55F_67L_ | 3.370355228 |
| 3645 | 6546 | CTGTTTTGCCCCATCTGCGGCTTTCACTGTactcagaaaggtaacctcctccgccacattaaaCTG | 46L_47F_49P_50I_52G_53F_54H_55C_67L_ | 3.366045284 |
| 3646 | 4201 | TTCTGGTGTAACATCTGTGGGGCATGTTTTactcagaaaggtaacctcctccgccacattaaaCTG | 46F_47W_49N_50I_52G_53A_54C_55F_67L_ | 3.356166958 |
| 3647 | 974 | TTCTTTTGTGACATTTGTGGGGCAAGATTTactcagaaaggtaacctcctccgccacattaaaCTG | 46F_47F_49D_50I_52G_53A_54R_55F_67L_ | 3.354599613 |
| 3648 | 4334 | TTCTATTGTGATATCTGTGGGGCAAGATTTactcagaaaggtaacctcctccgccacattaaaCTG | 46F_47Y_49D_50I_52G_53A_54R_55F_67L_ | 3.351411015 |
| 3649 | 1154 | TTCTTTTGTCCAATCTGTGGGGCACGCTTTactcagaaaggtaacctcctccgccacattaaaCTG | 46F_47F_49P_50I_52G_53A_54R_55F_67L_ | 3.350195058 |
| 3650 | 4272 | TTCTGGTGTCCCATCTGTGGGGCACCGTTTactcagaaaggtaacctcctccgccacattaaaCTG | 46F_47W_49P_50I_52G_53A_54P_55F_67L_ | 3.349834426 |
| 3651 | 1032 | TTCTTTTGTGAAATCTGTGGGGCACCCTTTactcagaaaggtaacctcctccgccacattaaaCTG | 46F_47F_49E_50I_52G_53A_54P_55F_67L_ | 3.341298127 |
| 3652 | 2980 | TTCCAATGTGAAGTTTGTGGGGCAGCCTTTactcagaaaggtaacctcctccgccacattaaaCTG | 46F_47Q_49E_50V_52G_53A_54A_55F_67L_ | 3.341271822 |
| 3653 | 1181 | TTCTTCTGTCCAGTATGTGGGGCATGCTTTactcagaaaggtaacctcctccgccacattaaaCTG | 46F_47F_49P_50V_52G_53A_54C_55F_67L_ | 3.338541077 |
| 3654 | 3112 | TTCCAATGTCCCGTTTGTGGGGCACCTTTTactcagaaaggtaacctcctccgccacattaaaCTG | 46F_47Q_49P_50V_52G_53A_54P_55F_67L_ | 3.336851711 |
| 3655 | 4214 | TTCTGGTGTAACATTTGTGGGGCAAGATTTactcagaaaggtaacctcctccgccacattaaaCTG | 46F_47W_49N_50I_52G_53A_54R_55F_67L_ | 3.335690198 |
| 3656 | 4072 | TTCGTTTGTCCGGTCTGTGGGGCACCATTTactcagaaaggtaacctcctccgccacattaaaCTG | 46F_47V_49P_50V_52G_53A_54P_55F_67L_ | 3.332685227 |
| 3657 | 6820 | CTCTACTGTCCGATCTGTGGCTTTGCTTGTactcagaaaggtaacctcctccgccacattaaaCTG | 46L_47Y_49P_50I_52G_53F_54A_55C_67L_ | 3.33180872 |
| 3658 | 1026 | TTCTTTTGTGAGATCTGTGGGGCACACTTTactcagaaaggtaacctcctccgccacattaaaCTG | 46F_47F_49E_50I_52G_53A_54H_55F_67L_ | 3.329564783 |
| 3659 | 4328 | TTCTACTGTGATATTTGTGGGGCAAAGTTTactcagaaaggtaacctcctccgccacattaaaCTG | 46F_47Y_49D_50I_52G_53A_54K_55F_67L_ | 3.323740201 |
| 3660 | 3972 | TTCGTCTGTAACATTTGTGGGGCACCCTTTactcagaaaggtaacctcctccgccacattaaaCTG | 46F_47V_49N_50I_52G_53A_54P_55F_67L_ | 3.321899986 |
| 3661 | 6800 | CTATGGTGCCCCATCTGCGGCTTTGCCTGCactcagaaaggtaacctcctccgccacattaaaCTG | 46L_47W_49P_50I_52G_53F_54A_55C_67L_ | 3.321847454 |
| 3662 | 4200 | TTCTGGTGTAACATTTGTGGGGCAGCTTTTactcagaaaggtaacctcctccgccacattaaaCTG | 46F_47W_49N_50I_52G_53A_54A_55F_67L_ | 3.320582292 |
| 3663 | 6776 | CTGACGTGTCCCATTTGTGGGTTTACGTGTactcagaaaggtaacctcctccgccacattaaaCTG | 46L_47T_49P_50I_52G_53F_54T_55C_67L_ | 3.309871576 |
| 3664 | 7941 | CTATGGTGCGAAATATGCGGATTCTGTTGCactcagaaaggtaacctcctccgccacattaaaCTG | 46L_47W_49E_50I_52G_53F_54C_55C_67L_ | 3.308436464 |
| 3665 | 4261 | TTCTGGTGTCCAATCTGTGGGGCATGCTTTactcagaaaggtaacctcctccgccacattaaaCTG | 46F_47W_49P_50I_52G_53A_54C_55F_67L_ | 3.307646807 |
| 3666 | 4260 | TTCTGGTGTCCCATATGTGGGGCAGCTTTTactcagaaaggtaacctcctccgccacattaaaCTG | 46F_47W_49P_50I_52G_53A_54A_55F_67L_ | 3.303476956 |
| 3667 | 3172 | TTCAGATGTGACGTTTGTGGGGCACCGTTTactcagaaaggtaacctcctccgccacattaaaCTG | 46F_47R_49D_50V_52G_53A_54P_55F_67L_ | 3.298396302 |
| 3668 | 7616 | TTAGCCTGCGAGATCTGCGGGTTTACCTGTactcagaaaggtaacctcctccgccacattaaaCTG | 46L_47A_49E_50I_52G_53F_54T_55C_67L_ | 3.297108716 |
| 3669 | 4555 | TTCTACTGTCCCGTGTGTGGGGCAAGCTTTactcagaaaggtaacctcctccgccacattaaaCTG | 46F_47Y_49P_50V_52G_53A_54S_55F_67L_ | 3.296247241 |
| 3670 | 6553 | TTATTCTGCCCTATCTGTGGCTTTCAGTGCactcagaaaggtaacctcctccgccacattaaaCTG | 46L_47F_49P_50I_52G_53F_54Q_55C_67L_ | 3.294917969 |
| 3671 | 7977 | CTGTACTGCGAAATTTGCGGCTTTGTGTGTactcagaaaggtaacctcctccgccacattaaaCTG | 46L_47Y_49E_50I_52G_53F_54V_55C_67L_ | 3.292242023 |
| 3672 | 3852 | TTCGTCTGTGATATTTGTGGGGCACCATTTactcagaaaggtaacctcctccgccacattaaaCTG | 46F_47V_49D_50I_52G_53A_54P_55F_67L_ | 3.291565693 |
| 3673 | 1194 | TTCTTCTGTCCTGTGTGTGGGGCACGATTTactcagaaaggtaacctcctccgccacattaaaCTG | 46F_47F_49P_50V_52G_53A_54R_55F_67L_ | 3.290268025 |
| 3674 | 4308 | TTCTGGTGTCCAGTGTGTGGGGCAAAGTTTactcagaaaggtaacctcctccgccacattaaaCTG | 46F_47W_49P_50V_52G_53A_54K_55F_67L_ | 3.288516821 |
| 3675 | 1092 | TTCTTCTGTAACATCTGTGGGGCACCTTTTactcagaaaggtaacctcctccgccacattaaaCTG | 46F_47F_49N_50I_52G_53A_54P_55F_67L_ | 3.287102763 |
| 3676 | 973 | TTCTTTTGTGACATTTGTGGGGCACAATTTactcagaaaggtaacctcctccgccacattaaaCTG | 46F_47F_49D_50I_52G_53A_54Q_55F_67L_ | 3.2847154 |
| 3677 | 4374 | TTCTACTGTGACGTGTGTGGGGCACGTTTTactcagaaaggtaacctcctccgccacattaaaCTG | 46F_47Y_49D_50V_52G_53A_54R_55F_67L_ | 3.284061451 |
| 3678 | 7961 | TTGTACTGTGAGATCTGCGGATTCTGTTGTactcagaaaggtaacctcctccgccacattaaaCTG | 46L_47Y_49E_50I_52G_53F_54C_55C_67L_ | 3.281802585 |
| 3679 | 3940 | TTCGTCTGTGAAGTGTGTGGGGCAGCATTTactcagaaaggtaacctcctccgccacattaaaCTG | 46F_47V_49E_50V_52G_53A_54A_55F_67L_ | 3.275279183 |
| 3680 | 4512 | TTCTATTGTCCAATATGTGGGGCACCGTTTactcagaaaggtaacctcctccgccacattaaaCTG | 46F_47Y_49P_50I_52G_53A_54P_55F_67L_ | 3.274464928 |
| 3681 | 4384 | TTCTATTGTGAGATCTGTGGGGCATTTTTTactcagaaaggtaacctcctccgccacattaaaCTG | 46F_47Y_49E_50I_52G_53A_54F_55F_67L_ | 3.271423769 |
| 3682 | 7970 | CTCTATTGTGAGATCTGTGGATTTATGTGTactcagaaaggtaacctcctccgccacattaaaCTG | 46L_47Y_49E_50I_52G_53F_54M_55C_67L_ | 3.268958413 |
| 3683 | 4383 | TTCTACTGTGAAATCTGTGGGGCAGAGTTTactcagaaaggtaacctcctccgccacattaaaCTG | 46F_47Y_49E_50I_52G_53A_54E_55F_67L_ | 3.263389941 |
| 3684 | 3067 | TTCCAGTGTCCTATCTGTGGGGCAATTTTTactcagaaaggtaacctcctccgccacattaaaCTG | 46F_47Q_49P_50I_52G_53A_54I_55F_67L_ | 3.259428163 |
| 3685 | 7967 | CTCTACTGCGAGATCTGTGGGTTCATATGCactcagaaaggtaacctcctccgccacattaaaCTG | 46L_47Y_49E_50I_52G_53F_54I_55C_67L_ | 3.256214155 |
| 3686 | 6780 | CTCGTTTGCCCAATTTGTGGGTTTGCTTGTactcagaaaggtaacctcctccgccacattaaaCTG | 46L_47V_49P_50I_52G_53F_54A_55F_67L_ | 3.252862166 |
| 3687 | 2412 | TTCATGTGTGACATTTGTGGGGCACCCTTTactcagaaaggtaacctcctccgccacattaaaCTG | 46F_47M_49D_50I_52G_53A_54P_55F_67L_ | 3.248990988 |
| 3688 | 7948 | CTGTGGTGCGAAATCTGCGGATTCAAGTGTactcagaaaggtaacctcctccgccacattaaaCTG | 46L_47W_49E_50I_52G_53F_54K_55C_67L_ | 3.248030046 |
| 3689 | 3712 | TTCACATGTGAGGTATGTGGGGCACCTTTTactcagaaaggtaacctcctccgccacattaaaCTG | 46F_47T_49E_50V_52G_53A_54P_55F_67L_ | 3.247614129 |
| 3690 | 4500 | TTCTACTGTCCAATCTGTGGGGCAGCATTTactcagaaaggtaacctcctccgccacattaaaCTG | 46F_47Y_49P_50I_52G_53A_54A_55F_67L_ | 3.239476414 |
| 3691 | 4552 | TTCTATTGTCCAGTGTGTGGGGCACCTTTTactcagaaaggtaacctcctccgccacattaaaCTG | 46F_47Y_49P_50V_52G_53A_54P_55F_67L_ | 3.238593885 |
| 3692 | 3700 | TTCACATGTGAAGTGTGTGGGGCAGCTTTTactcagaaaggtaacctcctccgccacattaaaCTG | 46F_47T_49E_50V_52G_53A_54A_55F_67L_ | 3.237683798 |
| 3693 | 4373 | TTCTACTGTGACGTGTGTGGGGCACAGTTTactcagaaaggtaacctcctccgccacattaaaCTG | 46F_47Y_49D_50V_52G_53A_54Q_55F_67L_ | 3.236120732 |
| 3694 | 7940 | TTATGGTGTGAGATCTGTGGCTTTGCCTGTactcagaaaggtaacctcctccgccacattaaaCTG | 46L_47W_49E_50I_52G_53F_54A_55C_67L_ | 3.236053003 |
| 3695 | 6476 | TTAGCCTGTCCCATTTGCGGGTTTACATGCactcagaaaggtaacctcctccgccacattaaaCTG | 46L_47A_49P_50I_52G_53F_54T_55C_67L_ | 3.232344022 |
| 3696 | 6819 | CTGTGGTGTCCCATCTGCGGCTTTTATTGCactcagaaaggtaacctcctccgccacattaaaCTG | 46L_47W_49P_50I_52G_53F_54Y_55C_67L_ | 3.230998109 |
| 3697 | 971 | TTCTTTTGTGATATCTGTGGGGCAAATTTTactcagaaaggtaacctcctccgccacattaaaCTG | 46F_47F_49D_50I_52G_53A_54N_55F_67L_ | 3.22805251 |
| 3698 | 4320 | TTCTACTGTGACATATGTGGGGCAGCCTTTactcagaaaggtaacctcctccgccacattaaaCTG | 46F_47Y_49D_50I_52G_53A_54A_55F_67L_ | 3.226692597 |
| 3699 | 4425 | TTCTACTGTGAGGTGTGTGGGGCAGGATTTactcagaaaggtaacctcctccgccacattaaaCTG | 46F_47Y_49E_50V_52G_53A_54G_55F_67L_ | 3.222835506 |
| 3700 | 7915 | TTGACTTGCGAGATATGTGGATTCTCCTGCactcagaaaggtaacctcctccgccacattaaaCTG | 46L_47T_49E_50I_52G_53F_54S_55C_67L_ | 3.215853176 |
| 3701 | 3672 | TTCACGTGTGAGATCTGTGGGGCACCCTTTactcagaaaggtaacctcctccgccacattaaaCTG | 46F_47T_49E_50I_52G_53A_54P_55F_67L_ | 3.214134996 |
| 3702 | 3660 | TTCACGTGTGAAATTTGTGGGGCAGCTTTTactcagaaaggtaacctcctccgccacattaaaCTG | 46F_47T_49E_50I_52G_53A_54A_55F_67L_ | 3.207746022 |
| 3703 | 3460 | TTCAGCTGTGAGGTCTGTGGGGCAGCTTTTactcagaaaggtaacctcctccgccacattaaaCTG | 46F_47S_49E_50V_52G_53A_54A_55F_67L_ | 3.206583873 |
| 3704 | 4060 | TTCGTGTGTCCTGTCTGTGGGGCAGCGTTTactcagaaaggtaacctcctccgccacattaaaCTG | 46F_47V_49P_50V_52G_53A_54A_55F_67L_ | 3.204408594 |
| 3705 | 2832 | TTCAACTGTCCTATTTGTGGGGCACCTTTTactcagaaaggtaacctcctccgccacattaaaCTG | 46F_47N_49P_50I_52G_53A_54P_55F_67L_ | 3.202616069 |
| 3706 | 7950 | CTCTGGTGTGAGATATGCGGGTTTATGTGCactcagaaaggtaacctcctccgccacattaaaCTG | 46L_47W_49E_50I_52G_53F_54M_55C_67L_ | 3.202360748 |
| 3707 | 4126 | TTCTGGTGTGATGTCTGTGGGGCACACTTTactcagaaaggtaacctcctccgccacattaaaCTG | 46F_47W_49D_50V_52G_53A_54H_55F_67L_ | 3.202145853 |
| 3708 | 4128 | TTCTGGTGTGATGTCTGTGGGGCAAAGTTTactcagaaaggtaacctcctccgccacattaaaCTG | 46F_47W_49D_50V_52G_53A_54K_55F_67L_ | 3.202109526 |
| 3709 | 6554 | CTGTTTTGCCCTATTTGTGGATTCCGCTGCactcagaaaggtaacctcctccgccacattaaaCTG | 46L_47F_49P_50I_52G_53F_54R_55C_67L_ | 3.202099403 |
| 3710 | 4335 | TTCTACTGTGATATTTGTGGGGCAAGCTTTactcagaaaggtaacctcctcogccacattaaaCTG | 46F_47Y_49D_50I_52G_53A_54S_55F_67L_ | 3.201965665 |
| 3711 | 4206 | TTCTGGTGTAATATCTGTGGGGCACACTTTactcagaaaggtaacctcctccgccacattaaaCTG | 46F_47W_49N_50I_52G_53A_54H_55F_67L_ | 3.193340812 |
| 3712 | 1072 | TTCTTCTGTGAGGTGTGTGGGGCACCCTTTactcagaaaggtaacctcctccgccacattaaaCTG | 46F_47F_49E_50V_52G_53A_54P_55F_67L_ | 3.183949552 |
| 3713 | 1195 | TTCTTCTGTCCCGTTTGTGGGGCATCCTTTactcagaaaggtaacctcctccgccacattaaaCTG | 46F_47F_49P_50V_52G_53A_54S_55F_67L_ | 3.181321088 |
| 3714 | 6475 | TTGGCGTGTCCAATTTGCGGATTCTCATGTactcagaaaggtaacctcctccgccacattaaaCTG | 46L_47A_49P_50I_52G_53F_54S_55C_67L_ | 3.181090965 |
| 3715 | 6808 | CTGTGGTGTCCTATCTGTGGGTTTAAGTGTactcagaaaggtaacctcctccgccacattaaaCTG | 46L_47W_49P_50I_52G_53F_54K_55C_67L_ | 3.179324107 |
| 3716 | 6785 | TTGGTGTGCCCCATATGTGGGTTCGGGTGTactcagaaaggtaacctcctccgccacattaaaCTG | 46L_47V_49P_50I_52G_53F_54G_55C_67L_ | 3.179034972 |
| 3717 | 1155 | TTCTTCTGTCCCATCTGTGGGGCATCCTTTactcagaaaggtaacctcctccgccacattaaaCTG | 46F_47F_49P_50I_52G_53A_54S_55F_67L_ | 3.178789816 |
| 3718 | 4385 | TTCTATTGTGAGATATGTGGGGCAGGGTTTactcagaaaggtaacctcctccgccacattaaaCTG | 46F_47Y_49E_50I_52G_53A_54G_55F_67L_ | 3.17415176 |
| 3719 | 7957 | CTGTGGTGCGAGATTTGTGGATTTGTATGCactcagaaaggtaacctcctccgccacattaaaCTG | 46L_47W_49E_50I_52G_53F_54V_55C_67L_ | 3.170038074 |
| 3720 | 1552 | TTCCACTGTGAGGTGTGTGGGGCACCTTTTactcagaaaggtaacctcctccgccacattaaaCTG | 46F_47H_49E_50V_52G_53A_54P_55F_67L_ | 3.169014419 |
| 3721 | 1421 | TTCGGATGTCCCGTCTGTGGGGCATGCTTTactcagaaaggtaacctcctccgccacattaaaCTG | 46F_47G_49P_50V_52G_53A_54C_55F_67L_ | 3.164859276 |
| 3722 | 1013 | TTCTTTTGTGACGTTTGTGGGGCACAATTTactcagaaaggtaacctcctccgccacattaaaCTG | 46F_47F_49D_50V_52G_53A_54Q_55F_67L_ | 3.163025891 |
| 3723 | 432 | TTCTGCTGTCCGATCTGTGGGGCACCTTTTactcagaaaggtaacctcctccgccacattaaaCTG | 46F_47C_49P_50I_52G_53A_54P_55F_67L_ | 3.162663518 |
| 3724 | 7735 | CTGCACTGTGAAATATGCGGCTTTTCATGCactcagaaaggtaacctcctccgccacattaaaCTG | 46L_47H_49E_50I_52G_53F_54S_55C_67L_ | 3.159758634 |
| 3725 | 3832 | TTCACTTGTCCCGTCTGTGGGGCACCGTTTactcagaaaggtaacctcctccgccacattaaaCTG | 46F_47T_49P_50V_52G_53A_54P_55F_67L_ | 3.157631063 |
| 3726 | 2947 | TTCCAATGTGAGATCTGTGGGGCAATCTTTactcagaaaggtaacctcctccgccacattaaaCTG | 46F_47Q_49E_50I_52G_53A_54I_55F_67L_ | 3.157512954 |
| 3727 | 972 | TTCTTCTGTGACATATGTGGGGCACCCTTTactcagaaaggtaacctcctccgccacattaaaCTG | 46F_47F_49D_50I_52G_53A_54P_55F_67L_ | 3.157498116 |
| 3728 | 7968 | TTATACTGCGAGATATGCGGCTTCAAATGCactcagaaaggtaacctcctccgccacattaaaCTG | 46L_47Y_49E_50I_52G_53F_54K_55C_67L_ | 3.157005232 |
| 3729 | 4253 | TTCTGGTGTAACGTATGTGGGGCACAGTTTactcagaaaggtaacctcctccgccacattaaaCTG | 46F_47W_49N_50V_52G_53A_54Q_55F_67L_ | 3.156102837 |
| 3730 | 3077 | TTCCAGTGTCCCATCTGTGGGGCAGTGTTTactcagaaaggtaacctcctccgccacattaaaCTG | 46F_47Q_49P_50I_52G_53A_54V_55F_67L_ | 3.156007962 |
| 3731 | 7688 | TTATTTTGCGAGATTTGTGGGTTTAAATGCactcagaaaggtaacctcctccgccacattaaaCTG | 46L_47F_49E_50I_52G_53F_54K_55C_67L_ | 3.153840344 |
| 3732 | 4371 | TTCTATTGTGATGTGTGTGGGGCAAACTTTactcagaaaggtaacctcctccgccacattaaaCTG | 46F_47Y_49D_50V_52G_53A_54N_55F_67L_ | 3.15161888 |
| 3733 | 4441 | TTCTATTGTAACATATGTGGGGCATGTTTTactcagaaaggtaacctcctccgccacattaaaCTG | 46F_47Y_49N_50I_52G_53A_54C_55F_67L_ | 3.151380122 |
| 3734 | 1014 | TTCTTTTGTGACGTGTGTGGGGCACGTTTTactcagaaaggtaacctcctccgccacattaaaCTG | 46F_47F_49D_50V_52G_53A_54R_55F_67L_ | 3.151195133 |
| 3735 | 968 | TTCTTTTGTGATATCTGTGGGGCAAAGTTTactcagaaaggtaacctcctccgccacattaaaCTG | 46F_47F_49D_50I_52G_53A_54K_55F_67L_ | 3.148146456 |
| 3736 | 6817 | CTCTGGTGCCCCATCTGCGGATTCGTGTGCactcagaaaggtaacctcctccgccacattaaaCTG | 46L_47W_49P_50I_52G_53F_54V_55C_67L_ | 3.1480596 |
| 3737 | 3107 | TTCCAGTGTCCAGTGTGTGGGGCAATTTTTactcagaaaggtaacctcctccgccacattaaaCTG | 46F_47Q_49P_50V_52G_53A_54I_55F_67L_ | 3.147648379 |
| 3738 | 4453 | TTCTATTGTAATATCTGTGGGGCACAGTTTactcagaaaggtaacctcctccgccacattaaaCTG | 46F_47Y_49N_50I_52G_53A_54Q_55F_67L_ | 3.14763838 |
| 3739 | 6495 | CTCTGCTGCCCCATCTGTGGTTTCAGTTGCactcagaaaggtaacctcctccgccacattaaaCTG | 46L_47C_49P_50I_52G_53F_54S_55C_67L_ | 3.147180402 |
| 3740 | 1860 | TTCATTTGTCCTATATGTGGGGCAGCCTTTactcagaaaggtaacctcctccgccacattaaaCTG | 46F_47I_49P_50I_52G_53A_54A_55F_67L_ | 3.146911724 |
| 3741 | 4187 | TTCTGGTGTGAAGTCTGTGGGGCAATCTTTactcagaaaggtaacctcctccgccacattaaaCTG | 46F_47W_49E_50V_52G_53A_54I_55F_67L_ | 3.146517914 |
| 3742 | 1070 | TTCTTTTGTGAAGTGTGTGGGGCAATGTTTactcagaaaggtaacctcctccgccacattaaaCTG | 46F_47F_49E_50V_52G_53A_54M_55F_67L_ | 3.145055397 |
| 3743 | 961 | TTCTTTTGTGATATCTGTGGGGCATGTTTTactcagaaaggtaacctcctccgccacattaaaCTG | 46F_47F_49D_50I_52G_53A_54C_55F_67L_ | 3.142921226 |
| 3744 | 2892 | TTCCAGTGTGATATTTGTGGGGCACCCTTTactcagaaaggtaacctcctccgccacattaaaCTG | 46F_47Q_49D_50I_52G_53A_54P_55F_67L_ | 3.140796134 |
| 3745 | 4291 | TTCTGGTGTCCCCAGTGTGGGGCAAACTTTactcagaaaggtaacctcctccgccacattaaaCTG | 46F_47W_49P_50Q_52G_53A_54N_55F_67L_ | 3.137579392 |
| 3746 | 3060 | TTCCAATGTCCTATATGTGGGGCAGCTTTTactcagaaaggtaacctcctccgccacattaaaCTG | 46F_47Q_49P_50I_52G_53A_54A_55F_67L_ | 3.129082789 |
| 3747 | 4144 | TTCTGGTGTGAAATTTGTGGGGCATTCTTTactcagaaaggtaacctcctccgccacattaaaCTG | 46F_47W_49E_50I_52G_53A_54F_55F_67L_ | 3.128878247 |
| 3748 | 6760 | CTAACCTGCCCAATCTGCGGGTTCGCTTGTactcagaaaggtaacctcctccgccacattaaaCTG | 46L_47T_49P_50I_52G_53F_54A_55C_67L_ | 3.124761731 |
| 3749 | 1066 | TTCTTCTGTGAGGTTTGTGGGGCACACTTTactcagaaaggtaacctcctccgccacattaaaCTG | 46F_47F_49E_50V_52G_53A_54H_55F_67L_ | 3.12366761 |
| 3750 | 3674 | TTCACCTGTGAAATTTGTGGGGCAAGGTTTactcagaaaggtaacctcctccgccacattaaaCTG | 46F_47T_49E_50I_52G_53A_54R_55F_67L_ | 3.122794606 |
| 3751 | 7686 | CTGTTTTGCGAAATCTGCGGATTTCATTGTactcagaaaggtaacctcctccgccacattaaaCTG | 46L_47F_49E_50I_52G_53F_54H_55C_67L_ | 3.122508491 |
| 3752 | 1030 | TTCTTTTGTGAGATCTGTGGGGCAATGTTTactcagaaaggtaacctcctccgccacattaaaCTG | 46F_47F_49E_50I_52G_53A_54M_55F_67L_ | 3.121050672 |
| 3753 | 4174 | TTCTGGTGTGAACAGTGTGGGGCACGCTTTactcagaaaggtaacctcctccgccacattaaaCTG | 46F_47W_49E_50Q_52G_53A_54R_55F_67L_ | 3.120903125 |
| 3754 | 4375 | TTCTATTGTGATGTCTGTGGGGCATCCTTTactcagaaaggtaacctcctccgccacattaaaCTG | 46F_47Y_49D_50V_52G_53A_54S_55F_67L_ | 3.118482562 |
| 3755 | 7690 | TTATTCTGTGAGATCTGTGGATTTATGTGCactcagaaaggtaacctcctccgccacattaaaCTG | 46L_47F_49E_50I_52G_53F_54M_55C_67L_ | 3.117068351 |
| 3756 | 4519 | TTCTACTGTCCAATATGTGGGGCATACTTTactcagaaaggtaacctcctccgccacattaaaCTG | 46F_47Y_49P_50I_52G_53A_54Y_55F_67L_ | 3.1160603 |
| 3757 | 1023 | TTCTTCTGTGAAATATGTGGGGCAGAGTTTactcagaaaggtaacctcctccgccacattaaaCTG | 46F_47F_49E_50I_52G_53A_54E_55F_67L_ | 3.113861335 |
| 3758 | 7935 | TTAGTGTGCGAAATCTGCGGCTTCTCCTGCactcagaaaggtaacctcctccgccacattaaaCTG | 46L_47V_49E_50I_52G_53F_54S_55C_67L_ | 3.11296702 |
| 3759 | 3820 | TTCACCTGTCCCGTGTGTGGGGCAGCATTTactcagaaaggtaacctcctccgccacattaaaCTG | 46F_47T_49P_50V_52G_53A_54A_55F_67L_ | 3.111241828 |
| 3760 | 6839 | CTCTATTGTCCAATCTGCGGTTTTTATTGCactcagaaaggtaacctcctccgccacattaaaCTG | 46L_47Y_49P_50I_52G_53F_54Y_55C_67L_ | 3.109764728 |
| 3761 | 3117 | TTCCAATGTCCCGTATGTGGGGCAGTTTTTactcagaaaggtaacctcctccgccacattaaaCTG | 46F_47Q_49P_50V_52G_53A_54V_55F_67L_ | 3.105818222 |
| 3762 | 4414 | TTCTACTGTGAGCAGTGTGGGGCACGCTTTactcagaaaggtaacctcctccgccacattaaaCTG | 46F_47Y_49E_50Q_52G_53A_54R_55F_67L_ | 3.103846164 |
| 3763 | 3100 | TTCCAGTGTCCAGTTTGTGGGGCAGCCTTTactcagaaaggtaacctcctccgccacattaaaCTG | 46F_47Q_49P_50V_52G_53A_54A_55F_67L_ | 3.101770463 |
| 3764 | 300 | TTCTGCTGTGAAATCTGTGGGGCAGCCTTTactcagaaaggtaacctcctccgccacattaaaCTG | 46F_47C_49E_50I_52G_53A_54A_55F_67L_ | 3.101072486 |
| 3765 | 3012 | TTCCAATGTAATATATGTGGGGCACCCTTTactcagaaaggtaacctcctccgccacattaaaCTG | 46F_47Q_49N_50I_52G_53A_54P_55F_67L_ | 3.090141977 |
| 3766 | 4294 | TTCTGGTGTCCTCAATGTGGGGCACGTTTTactcagaaaggtaacctcctccgccacattaaaCTG | 46F_47W_49P_50Q_52G_53A_54R_55F_67L_ | 3.084510179 |
| 3767 | 4025 | TTCGTCTGTCCAATATGTGGGGCAGGCTTTactcagaaaggtaacctcctccgccacattaaaCTG | 46F_47V_49P_50I_52G_53A_54G_55F_67L_ | 3.084509917 |
| 3768 | 1732 | TTCATTTGTGATGTCTGTGGGGCACCCTTTactcagaaaggtaacctcctccgccacattaaaCTG | 46F_47I_49D_50V_52G_53A_54P_55F_67L_ | 3.083290936 |
| 3769 | 2941 | TTCCAATGTGAAATCTGTGGGGCATGTTTTactcagaaaggtaacctcctccgccacattaaaCTG | 46F_47Q_49E_50I_52G_53A_54C_55F_67L_ | 3.081611211 |
| 3770 | 6615 | TTGATCTGCCCAATTTGCGGCTTCTCCTGCactcagaaaggtaacctcctccgccacattaaaCTG | 46L_47I_49P_50I_52G_53F_54S_55C_67L_ | 3.075867447 |
| 3771 | 3905 | TTCGTGTGTGAAATTTGTGGGGCAGGTTTTactcagaaaggtaacctcctccgccacattaaaCTG | 46F_47V_49E_50I_52G_53A_54G_55F_67L_ | 3.07260522 |
| 3772 | 4508 | TTCTATTGTCCCATCTGTGGGGCAAAGTTTactcagaaaggtaacctcctccgccacattaaaCTG | 46F_47Y_49P_50I_52G_53A_54K_55F_67L_ | 3.067391748 |
| 3773 | 2987 | TTCCAGTGTGAGGTCTGTGGGGCAATATTTactcagaaaggtaacctcctccgccacattaaaCTG | 46F_47Q_49E_50V_52G_53A_54I_55F_67L_ | 3.066597572 |
| 3774 | 3061 | TTCCAGTGTCCTATCTGTGGGGCATGTTTTactcagaaaggtaacctcctccgccacattaaaCTG | 46F_47Q_49P_50I_52G_53A_54C_55F_67L_ | 3.056902573 |
| 3775 | 4096 | TTCTGGTGTGATATCTGTGGGGCAACATTTactcagaaaggtaacctcctccgccacattaaaCTG | 46F_47W_49D_50I_52G_53A_54T_55F_67L_ | 3.054096342 |
| 3776 | 4554 | TTCTACTGTCCCGTGTGTGGGGCAAGGTTTactcagaaaggtaacctcctccgccacattaaaCTG | 46F_47Y_49P_50V_52G_53A_54R_55F_67L_ | 3.051984869 |
| 3777 | 4310 | TTCTGGTGTCCAGTCTGTGGGGCAATGTTTactcagaaaggtaacctcctccgccacattaaaCTG | 46F_47W_49P_50V_52G_53A_54M_55F_67L_ | 3.05076198 |
| 3778 | 2532 | TTCATGTGTAATATTTGTGGGGCACCCTTTactcagaaaggtaacctcctccgccacattaaaCTG | 46F_47M_49N_50I_52G_53A_54P_55F_67L_ | 3.04834723 |
| 3779 | 1063 | TTCTTCTGTGAGGTATGTGGGGCAGAGTTTactcagaaaggtaacctcctccgccacattaaaCTG | 46F_47F_49E_50V_52G_53A_54E_55F_67L_ | 3.046464293 |
| 3780 | 6755 | CTTTCGTGTCCCATTTGCGGTTTCTCATGCactcagaaaggtaacctcctccgccacattaaaCTG | 46L_47S_49P_50I_52G_53F_54S_55C_67L_ | 3.045361077 |
| 3781 | 6804 | CTATGGTGTCCAATCTGCGGCTTTTTTTGCactcagaaaggtaacctcctccgccacattaaaCTG | 46L_47W_49P_50I_52G_53F_54F_55C_67L_ | 3.044015137 |
| 3782 | 7687 | CTCTTCTGCGAAATCTGCGGGTTCATATGTactcagaaaggtaacctcctccgccacattaaaCTG | 46L_47F_49E_50I_52G_53F_54I_55C_67L_ | 3.041832033 |
| 3783 | 3714 | TTCACATGTGAGGTGTGTGGGGCAAGATTTactcagaaaggtaacctcctccgccacattaaaCTG | 46F_47T_49E_50V_52G_53A_54R_55F_67L_ | 3.041575504 |
| 3784 | 6551 | CTGTTTTGTCCCATTTGCGGGTTCAATTGCactcagaaaggtaacctcctccgccacattaaaCTG | 46L_47F_49P_50I_52G_53F_54N_55C_67L_ | 3.038414639 |
| 3785 | 114 | TTCGCCTGTGAGGTGTGTGGGGCAAGATTTactcagaaaggtaacctcctccgccacattaaaCTG | 46F_47A_49E_50V_52G_53A_54R_55F_67L_ | 3.032765335 |
| 3786 | 192 | TTCGCTTGTCCCATTTGTGGGGCACCATTTactcagaaaggtaacctcctccgccacattaaaCTG | 46F_47A_49P_50I_52G_53A_54P_55F_67L_ | 3.03181003 |
| 3787 | 60 | TTCGCATGTGAGATCTGTGGGGCAGCCTTTactcagaaaggtaacctcctccgccacattaaaCTG | 46F_47A_49E_50I_52G_53A_54A_55F_67L_ | 3.030383674 |
| 3788 | 4033 | TTCGTGTGTCCGATTTGTGGGGCACAGTTTactcagaaaggtaacctcctccgccacattaaaCTG | 46F_47V_49P_50I_52G_53A_54Q_55F_67L_ | 3.030251173 |
| 3789 | 4429 | TTCTATTGTGAAGTGTGTGGGGCACTGTTTactcagaaaggtaacctcctccgccacattaaaCTG | 46F_47Y_49E_50V_52G_53A_54L_55F_67L_ | 3.029339262 |
| 3790 | 3675 | TTCACATGTGAAATATGTGGGGCAAGTTTTactcagaaaggtaacctcctccgccacattaaaCTG | 46F_47T_49E_50I_52G_53A_54S_55F_67L_ | 3.027913384 |
| 3791 | 3732 | TTCACATGTAATATCTGTGGGGCACCCTTTactcagaaaggtaacctoctocgccacattaaaCTG | 46F_47T_49N_50I_52G_53A_54P_55F_67L_ | 3.027105831 |
| 3792 | 4517 | TTCTACTGTCCTATTTGTGGGGCAGTGTTTactcagaaaggtaacctcctccgccacattaaaCTG | 46F_47Y_49P_50I_52G_53A_54V_55F_67L_ | 3.026740516 |
| 3793 | 7755 | CTTATCTGTGAGATATGCGGATTTTCCTGCactcagaaaggtaacctcctccgccacattaaaCTG | 46L_47I_49E_50I_52G_53F_54S_55C_67L_ | 3.024411366 |
| 3794 | 3795 | TTCACTTGTCCAATCTGTGGGGCAAGCTTTactcagaaaggtaacctcctccgccacattaaaCTG | 46F_47T_49P_50I_52G_53A_54S_55F_67L_ | 3.024337517 |
| 3795 | 354 | TTCTGTTGTGAGGTGTGTGGGGCACGTTTTactcagaaaggtaacctcctccgccacattaaaCTG | 46F_47C_49E_50V_52G_53A_54R_55F_67L_ | 3.022354757 |
| 3796 | 7680 | TTGTTTTGTGAGATCTGTGGTTTTGCGTGCactcagaaaggtaacctcctccgccacattaaaCTG | 46L_47F_49E_50I_52G_53F_54A_55C_67L_ | 3.019206009 |
| 3797 | 1067 | TTCTTCTGTGAGGTATGTGGGGCAATATTTactcagaaaggtaacctcctccgccacattaaaCTG | 46F_47F_49E_50V_52G_53A_54I_55F_67L_ | 3.016734313 |
| 3798 | 314 | TTCTGCTGTGAAATATGTGGGGCACGTTTTactcagaaaggtaacctcctccgccacattaaaCTG | 46F_47C_49E_50I_52G_53A_54R_55F_67L_ | 3.010734915 |
| 3799 | 4255 | TTCTGGTGTAACGTCTGTGGGGCATCTTTTactcagaaaggtaacctcctccgccacattaaaCTG | 46F_47W_49N_50V_52G_53A_54S_55F_67L_ | 3.009017474 |
| 3800 | 6794 | TTGGTCTGTCCAATTTGCGGGTTCCGATGTactcagaaaggtaacctcctccgccacattaaaCTG | 46L_47V_49P_50I_52G_53F_54R_55C_67L_ | 3.007967474 |
| 3801 | 1572 | TTCCATTGTAACATATGTGGGGCACCTTTTactcagaaaggtaacctcctccgccacattaaaCTG | 46F_47H_49N_50I_52G_53A_54P_55F_67L_ | 3.007696087 |
| 3802 | 1500 | TTCCACTGTGAAATATGTGGGGCAGCGTTTactcagaaaggtaacctcctccgccacattaaaCTG | 46F_47H_49E_50I_52G_53A_54A_55F_67L_ | 3.003100361 |
| 3803 | 3068 | TTCCAGTGTCCCATCTGTGGGGCAAAATTTactcagaaaggtaacctcctccgccacattaaaCTG | 46F_47Q_49P_50I_52G_53A_54K_55F_67L_ | 3.001140127 |
| 3804 | 1780 | TTCATATGTGAGGTCTGTGGGGCAGCGTTTactcagaaaggtaacctcctccgccacattaaaCTG | 46F_47I_49E_50V_52G_53A_54A_55F_67L_ | 3.000952877 |
| 3805 | 4264 | TTCTGGTGTCCAATCTGTGGGGCATTCTTTactcagaaaggtaacctcctccgccacattaaaCTG | 46F_47W_49P_50I_52G_53A_54F_55F_67L_ | 3.000294533 |
| 3806 | 1140 | TTCTTCTGTCCAATTTGTGGGGCAGCCTTTactcagaaaggtaacctcctccgccacattaaaCTG | 46F_47F_49P_50I_52G_53A_54A_55F_67L_ | 2.999243332 |
| 3807 | 6828 | CTGTATTGCCCTATTTGCGGTTTCAAGTGTactcagaaaggtaacctcctccgccacattaaaCTG | 46L_47Y_49P_50I_52G_53F_54K_55C_67L_ | 2.997907415 |
| 3808 | 100 | TTCGCCTGTGAGGTCTGTGGGGCAGCCTTTactcagaaaggtaacctcctccgccacattaaaCTG | 46F_47A_49E_50V_52G_53A_54A_55F_67L_ | 2.993009004 |
| 3809 | 6557 | CTGTTCTGTCCGATCTGTGGCTTCGTATGTactcagaaaggtaacctcctccgccacattaaaCTG | 46L_47F_49P_50I_52G_53F_54V_55C_67L_ | 2.992152803 |
| 3810 | 4317 | TTCTGGTGTCCCGTCTGTGGGGCAGTTTTTactcagaaaggtaacctcctccgccacattaaaCTG | 46F_47W_49P_50V_52G_53A_54V_55F_67L_ | 2.99107541 |
| 3811 | 975 | TTCTTCTGTGACATCTGTGGGGCAAGTTTTactcagaaaggtaacctcctccgccacattaaaCTG | 46F_47F_49D_50I_52G_53A_54S_55F_67L_ | 2.989348919 |
| 3812 | 4097 | TTCTGGTGTGACATATGTGGGGCAGTGTTTactcagaaaggtaacctcctccgccacattaaaCTG | 46F_47W_49D_50I_52G_53A_54V_55F_67L_ | 2.985802941 |
| 3813 | 4149 | TTCTGGTGTGAAATCTGTGGGGCACTGTTTactcagaaaggtaacctcctccgccacattaaaCTG | 46F_47W_49E_50I_52G_53A_54L_55F_67L_ | 2.985768432 |
| 3814 | 312 | TTCTGCTGTGAGATTTGTGGGGCACCTTTTactcagaaaggtaacctcctccgccacattaaaCTG | 46F_47C_49E_50I_52G_53A_54P_55F_67L_ | 2.985687681 |
| 3815 | 2988 | TTCCAATGTGAGGTGTGTGGGGCAAAGTTTactcagaaaggtaacctcctccgccacattaaaCTG | 46F_47Q_49E_50V_52G_53A_54K_55F_67L_ | 2.983048325 |
| 3816 | 1157 | TTCTTCTGTCCCATCTGTGGGGCAGTCTTTactcagaaaggtaacctcctccgccacattaaaCTG | 46F_47F_49P_50I_52G_53A_54V_55F_67L_ | 2.98014382 |
| 3817 | 2352 | TTCTTATGTCCCATCTGTGGGGCACCCTTTactcagaaaggtaacctcctccgccacattaaaCTG | 46F_47L_49P_50I_52G_53A_54P_55F_67L_ | 2.978874876 |
| 3818 | 6548 | CTGTTTTGTCCCATCTGTGGCTTCAAGTGTactcagaaaggtaacctcctccgccacattaaaCTG | 46L_47F_49P_50I_52G_53F_54K_55C_67L_ | 2.978114417 |
| 3819 | 4199 | TTCTGGTGTGAGGTCTGTGGGGCATACTTTactcagaaaggtaacctcctccgccacattaaaCTG | 46F_47W_49E_50V_52G_53A_54Y_55F_67L_ | 2.97695404 |
| 3820 | 4246 | TTCTGGTGTAACGTGTGTGGGGCACACTTTactcagaaaggtaacctcctccgccacattaaaCTG | 46F_47W_49N_50V_52G_53A_54H_55F_67L_ | 2.970151402 |
| 3821 | 3780 | TTCACGTGTCCAATCTGTGGGGCAGCTTTTactcagaaaggtaacctcctccgccacattaaaCTG | 46F_47T_49P_50I_52G_53A_54A_55F_67L_ | 2.967688724 |
| 3822 | 4440 | TTCTATTGTAATATCTGTGGGGCAGCCTTTactcagaaaggtaacctcctccgccacattaaaCTG | 46F_47Y_49N_50I_52G_53A_54A_55F_67L_ | 2.967271598 |
| 3823 | 6596 | CTGCACTGCCCTATCTGTGGCTTCACATGTactcagaaaggtaacctcctccgccacattaaaCTG | 46L_47H_49P_50I_52G_53F_54T_55C_67L_ | 2.967170702 |
| 3824 | 6825 | TTGTACTGTCCCATCTGTGGTTTCGGTTGCactcagaaaggtaacctcctccgccacattaaaCTG | 46L_47Y_49P_50I_52G_53F_54G_55C_67L_ | 2.963415918 |
| 3825 | 3715 | TTCACATGTGAAGTGTGTGGGGCATCGTTTactcagaaaggtaacctcctccgccacattaaaCTG | 46F_47T_49E_50V_52G_53A_54S_55F_67L_ | 2.960232545 |
| 3826 | 3292 | TTCAGATGTAATGTTTGTGGGGCACCATTTactcagaaaggtaacctcctccgccacattaaaCTG | 46F_47R_49N_50V_52G_53A_54P_55F_67L_ | 2.959006041 |
| 3827 | 7615 | CTGGCGTGTGAAATTTGTGGATTCAGTTGCactcagaaaggtaacctcctccgccacattaaaCTG | 46L_47A_49E_50I_52G_53F_54S_55C_67L_ | 2.958217044 |
| 3828 | 6700 | CTCCAGTGCCCGATTTGTGGGTTTGCTTGTactcagaaaggtaacctcctccgccacattaaaCTG | 46L_47Q_49P_50I_52G_53F_54A_55C_67L_ | 2.957784222 |
| 3829 | 340 | TTCTGTTGTGAAGTGTGTGGGGCAGCCTTTactcagaaaggtaacctcctccgccacattaaaCTG | 46F_47C_49E_50V_52G_53A_54A_55F_67L_ | 2.956628739 |
| 3830 | 4254 | TTCTGGTGTAACGTCTGTGGGGCACGTTTTactcagaaaggtaacctcctccgccacattaaaCTG | 46F_47W_49N_50V_52G_53A_54R_55F_67L_ | 2.956586432 |
| 3831 | 6433 | TTTTGGTGTCCAATATGCGGCCGGCAGTTTactcagaaaggtaacctcctccgccacattaaaCTG | 46F_47W_49P_50I_52G_53R_54Q_55F_67L_ | 2.956138592 |
| 3832 | 3794 | TTCACGTGTCCCATATGTGGGGCACGCTTTactcagaaaggtaacctcctccgccacattaaaCTG | 46F_47T_49P_50I_52G_53A_54R_55F_67L_ | 2.953140462 |
| 3833 | 2948 | TTCCAGTGTGAAATTTGTGGGGCAAAGTTTactcagaaaggtaacctcctccgccacattaaaCTG | 46F_47Q_49E_50I_52G_53A_54K_55F_67L_ | 2.953058509 |
| 3834 | 4423 | TTCTATTGTGAAGTTTGTGGGGCAGAGTTTactcagaaaggtaacctcctccgccacattaaaCTG | 46F_47Y_49E_50V_52G_53A_54E_55F_67L_ | 2.951075069 |
| 3835 | 1740 | TTCATTTGTGAAATCTGTGGGGCAGCATTTactcagaaaggtaacctcctccgccacattaaaCTG | 46F_47I_49E_50I_52G_53A_54A_55F_67L_ | 2.950910773 |
| 3836 | 6460 | CTAGCTTGTCCAATCTGCGGTTTTGCATGTactcagaaaggtaacctcctccgccacattaaaCTG | 46L_47A_49P_50I_52G_53F_54A_55C_67L_ | 2.950421262 |
| 3837 | 3420 | TTCAGCTGTGAGATATGTGGGGCAGCGTTTactcagaaaggtaacctcctccgccacattaaaCTG | 46F_47S_49E_50I_52G_53A_54A_55F_67L_ | 2.947927921 |
| 3838 | 4455 | TTCTATTGTAACATCTGTGGGGCATCCTTTactcagaaaggtaacctcctccgccacattaaaCTG | 46F_47Y_49N_50I_52G_53A_54S_55F_67L_ | 2.946425553 |
| 3839 | 6831 | CTTTATTGCCCAATCTGTGGGTTTAATTGCactcagaaaggtaacctcctccgccacattaaaCTG | 46L_47Y_49P_50I_52G_53F_54N_55C_67L_ | 2.946342788 |
| 3840 | 4360 | TTCTACTGTGACGTCTGTGGGGCAGCGTTTactcagaaaggtaacctcctccgccacattaaaCTG | 46F_47Y_49D_50V_52G_53A_54A_55F_67L_ | 2.940668807 |
| 3841 | 4173 | TTCTGGTGTGAGCAGTGTGGGGCACAATTTactcagaaaggtaacctcctccgccacattaaaCTG | 46F_47W_49E_50Q_52G_53A_54Q_55F_67L_ | 2.940297809 |
| 3842 | 180 | TTCGCATGTCCCATATGTGGGGCAGCTTTTactcagaaaggtaacctcctccgccacattaaaCTG | 46F_47A_49P_50I_52G_53A_54A_55F_67L_ | 2.934474985 |
| 3843 | 4509 | TTCTATTGTCCAATCTGTGGGGCATTGTTTactcagaaaggtaacctcctccgccacattaaaCTG | 46F_47Y_49P_50I_52G_53A_54L_55F_67L_ | 2.93369586 |
| 3844 | 4319 | TTCTGGTGTCCTGTTTGTGGGGCATACTTTactcagaaaggtaacctcctccgccacattaaaCTG | 46F_47W_49P_50V_52G_53A_54Y_55F_67L_ | 2.931696908 |
| 3845 | 7736 | TTGCATTGCGAAATATGTGGCTTCACTTGTactcagaaaggtaacctcctccgccacattaaaCTG | 46L_47H_49E_50I_52G_53F_54T_55C_67L_ | 2.929564684 |
| 3846 | 3892 | TTCGTGTGTGATGTATGTGGGGCACCCTTTactcagaaaggtaacctcctccgccacattaaaCTG | 46F_47V_49D_50V_52G_53A_54P_55F_67L_ | 2.929169115 |
| 3847 | 1261 | TTCGGCTGTGAAATCTGTGGGGCATGCTTTactcagaaaggtaacctcctccgccacattaaaCTG | 46F_47G_49E_50I_52G_53A_54C_55F_67L_ | 2.928445007 |
| 3848 | 1159 | TTCTTTTGTCCGATTTGTGGGGCATACTTTactcagaaaggtaacctcctccgccacattaaaCTG | 46F_47F_49P_50I_52G_53A_54Y_55F_67L_ | 2.925988439 |
| 3849 | 4208 | TTCTGGTGTAACATCTGTGGGGCAAAGTTTactcagaaaggtaacctcctccgccacattaaaCTG | 46F_47W_49N_50I_52G_53A_54K_55F_67L_ | 2.924927054 |
| 3850 | 4277 | TTCTGGTGTCCGATTTGTGGGGCAGTCTTTactcagaaaggtaacctcctccgccacattaaaCTG | 46F_47W_49P_50I_52G_53A_54V_55F_67L_ | 2.922373743 |
| 3851 | 4192 | TTCTGGTGTGAGGTGTGTGGGGCACCCTTTactcagaaaggtaacctcctccgccacattaaaCTG | 46F_47W_49E_50V_52G_53A_54P_55F_67L_ | 2.919461837 |
| 3852 | 4312 | TTCTGGTGTCCTGTGTGTGGGGCACCATTTactcagaaaggtaacctcctccgccacattaaaCTG | 46F_47W_49P_50V_52G_53A_54P_55F_67L_ | 2.918160436 |
| 3853 | 6830 | TTATATTGTCCTATCTGTGGATTCATGTGCactcagaaaggtaacctcctccgccacattaaaCTG | 46L_47Y_49P_50I_52G_53F_54M_55C_67L_ | 2.917440448 |
| 3854 | 4159 | TTCTGGTGTGAAATTTGTGGGGCATACTTTactcagaaaggtaacctcctccgccacattaaaCTG | 46F_47W_49E_50I_52G_53A_54Y_55F_67L_ | 2.916562198 |
| 3855 | 3552 | TTCTCATGTCCCATCTGTGGGGCACCGTTTactcagaaaggtaacctcctccgccacattaaaCTG | 46F_47S_49P_50I_52G_53A_54P_55F_67L_ | 2.916469244 |
| 3856 | 4184 | TTCTGGTGTGAGGTGTGTGGGGCATTCTTTactcagaaaggtaacctcctccgccacattaaaCTG | 46F_47W_49E_50V_52G_53A_54F_55F_67L_ | 2.916118219 |
| 3857 | 1148 | TTCTTTTGTCCCATATGTGGGGCAAAGTTTactcagaaaggtaacctcctccgccacattaaaCTG | 46F_47F_49P_50I_52G_53A_54K_55F_67L_ | 2.913646176 |
| 3858 | 3955 | TTCGTATGTGAGGTTTGTGGGGCATCCTTTactcagaaaggtaacctcctccgccacattaaaCTG | 46F_47V_49E_50V_52G_53A_54S_55F_67L_ | 2.911346425 |
| 3859 | 1180 | TTCTTCTGTCCCGTGTGTGGGGCAGCATTTactcagaaaggtaacctcctccgccacattaaaCTG | 46F_47F_49P_50V_52G_53A_54A_55F_67L_ | 2.907178918 |
| 3860 | 4336 | TTCTACTGTGACATCTGTGGGGCAACTTTTactcagaaaggtaacctcctccgccacattaaaCTG | 46F_47Y_49D_50I_52G_53A_54T_55F_67L_ | 2.907126285 |
| 3861 | 6740 | CTTAGCTGCCCCATCTGCGGTTTCGCCTGCactcagaaaggtaacctcctccgccacattaaaCTG | 46L_47S_49P_50I_52G_53F_54A_55C_67L_ | 2.90661232 |
| 3862 | 1029 | TTCTTCTGTGAAATTTGTGGGGCACTGTTTactcagaaaggtaacctcctccgccacattaaaCTG | 46F_47F_49E_50I_52G_53A_54L_55F_67L_ | 2.906594486 |
| 3863 | 4361 | TTCTACTGTGACGTTTGTGGGGCATGTTTTactcagaaaggtaacctcctccgccacattaaaCTG | 46F_47Y_49D_50V_52G_53A_54C_55F_67L_ | 2.898914494 |
| 3864 | 1025 | TTCTTCTGTGAAATTTGTGGGGCAGGGTTTactcagaaaggtaacctcctccgccacattaaaCTG | 46F_47F_49E_50I_52G_53A_54G_55F_67L_ | 2.895893562 |
| 3865 | 6827 | TTATACTGTCCAATTTGTGGTTTTATTTGCactcagaaaggtaacctcctccgccacattaaaCTG | 46L_47Y_49P_50I_52G_53F_54I_55C_67L_ | 2.895055266 |
| 3866 | 3332 | TTCAGATGTCCACAGTGTGGGGCACCATTTactcagaaaggtaacctcctccgccacattaaaCTG | 46F_47R_49P_50Q_52G_53A_54P_55F_67L_ | 2.894884069 |
| 3867 | 2995 | TTCCAGTGTGAGGTCTGTGGGGCATCCTTTactcagaaaggtaacctcctccgccacattaaaCTG | 46F_47Q_49E_50V_52G_53A_54S_55F_67L_ | 2.894785242 |
| 3868 | 3434 | TTCTCGTGTGAAATTTGTGGGGCACGTTTTactcagaaaggtaacctcctccgccacattaaaCTG | 46F_47S_49E_50I_52G_53A_54R_55F_67L_ | 2.894070791 |
| 3869 | 74 | TTCGCTTGTGAGATCTGTGGGGCACGATTTactcagaaaggtaacctcctccgccacattaaaCTG | 46F_47A_49E_50I_52G_53A_54R_55F_67L_ | 2.893535598 |
| 3870 | 3900 | TTCGTGTGTGAAATCTGTGGGGCAGCATTTactcagaaaggtaacctcctccgccacattaaaCTG | 46F_47V_49E_50I_52G_53A_54A_55F_67L_ | 2.893466037 |
| 3871 | 6352 | TTCAGGTGTCCAATATGTGGGCGGCCGTTCactcagaaaggtaacctcctccgccacattaaaCTG | 46F_47R_49P_50I_52G_53R_54P_55F_67L_ | 2.893087896 |
| 3872 | 4451 | TTCTACTGTAATATTTGTGGGGCAAACTTTactcagaaaggtaacctcctccgccacattaaaCTG | 46F_47Y_49N_50I_52G_53A_54N_55F_67L_ | 2.892788511 |
| 3873 | 3116 | TTCCAGTGTCCTGTATGTGGGGCAACTTTTactcagaaaggtaacctcctccgccacattaaaCTG | 46F_47Q_49P_50V_52G_53A_54T_55F_67L_ | 2.891391779 |
| 3874 | 7916 | CTGACCTGCGAAATCTGCGGATTTACTTGCactcagaaaggtaacctcctccgccacattaaaCTG | 46L_47T_49E_50I_52G_53F_54T_55C_67L_ | 2.891008247 |
| 3875 | 4241 | TTCTGGTGTAATGTGTGTGGGGCATGTTTTactcagaaaggtaacctcctccgccacattaaaCTG | 46F_47W_49N_50V_52G_53A_54C_55F_67L_ | 2.890515104 |
| 3876 | 6605 | CTCATCTGTCCTATCTGCGGGTTTGGATGCactcagaaaggtaacctcctccgccacattaaaCTG | 46L_47I_49P_50I_52G_53F_54G_55C_67L_ | 2.888767099 |
| 3877 | 6543 | CTGTTCTGCCCCATCTGCGGATTCGAGTGTactcagaaaggtaacctcctccgccacattaaaCTG | 46L_47F_49P_50I_52G_53F_54E_55C_67L_ | 2.888486929 |
| 3878 | 4399 | TTCTACTGTGAGATCTGTGGGGCATACTTTactcagaaaggtaacctcctccgccacattaaaCTG | 46F_47Y_49E_50I_52G_53A_54Y_55F_67L_ | 2.885672341 |
| 3879 | 1540 | TTCCATTGTGAAGTGTGTGGGGCAGCCTTTactcagaaaggtaacctcctccgccacattaaaCTG | 46F_47H_49E_50V_52G_53A_54A_55F_67L_ | 2.885540155 |
| 3880 | 3075 | TTCCAGTGTCCTATCTGTGGGGCAAGCTTTactcagaaaggtaacctcctccgccacattaaaCTG | 46F_47Q_49P_50I_52G_53A_54S_55F_67L_ | 2.884409126 |
| 3881 | 1795 | TTCATATGTGAGGTCTGTGGGGCATCGTTTactcagaaaggtaacctcctccgccacattaaaCTG | 46F_47I_49E_50V_52G_53A_54S_55F_67L_ | 2.880145321 |
| 3882 | 4145 | TTCTGGTGTGAAATCTGTGGGGCAGGCTTTactcagaaaggtaacctcctccgccacattaaaCTG | 46F_47W_49E_50I_52G_53A_54G_55F_67L_ | 2.876868197 |
| 3883 | 3108 | TTCCAGTGTCCCGTATGTGGGGCAAAGTTTactcagaaaggtaacctcctccgccacattaaaCTG | 46F_47Q_49P_50V_52G_53A_54K_55F_67L_ | 2.876596685 |
| 3884 | 6576 | CTGGGGTGTCCAATATGCGGATTTACCTGCactcagaaaggtaacctcctccgccacattaaaCTG | 46L_47G_49P_50I_52G_53F_54T_55C_67L_ | 2.873254733 |
| 3885 | 4504 | TTCTACTGTCCAATCTGTGGGGCATTTTTTactcagaaaggtaacctcctccgccacattaaaCTG | 46F_47Y_49P_50I_52G_53A_54F_55F_67L_ | 2.87313092 |
| 3886 | 220 | TTCGCCTGTCCTGTGTGTGGGGCAGCATTTactcagaaaggtaacctcctccgccacattaaaCTG | 46F_47A_49P_50V_52G_53A_54A_55F_67L_ | 2.873005833 |
| 3887 | 4136 | TTCTGGTGTGATGTCTGTGGGGCAACGTTTactcagaaaggtaacctcctccgccacattaaaCTG | 46F_47W_49D_50V_52G_53A_54T_55F_67L_ | 2.872960881 |
| 3888 | 1875 | TTCATCTGTCCGATTTGTGGGGCATCCTTTactcagaaaggtaacctcctccgccacattaaaCTG | 46F_47I_49P_50I_52G_53A_54S_55F_67L_ | 2.872935386 |
| 3889 | 4439 | TTCTACTGTGAAGTGTGTGGGGCATACTTTactcagaaaggtaacctcctccgccacattaaaCTG | 46F_47Y_49E_50V_52G_53A_54Y_55F_67L_ | 2.870428116 |
| 3890 | 4549 | TTCTATTGTCCGGTGTGTGGGGCATTGTTTactcagaaaggtaacctcctccgccacattaaaCTG | 46F_47Y_49P_50V_52G_53A_54L_55F_67L_ | 2.87019518 |
| 3891 | 7896 | CTGTCCTGCGAAATCTGCGGATTCACATGCactcagaaaggtaacctcctccgccacattaaaCTG | 46L_47S_49E_50I_52G_53F_54T_55C_67L_ | 2.86630954 |
| 3892 | 1672 | TTCCATTGTCCAGTGTGTGGGGCACCCTTTactcagaaaggtaacctcctccgccacattaaaCTG | 46F_47H_49P_50V_52G_53A_54P_55F_67L_ | 2.863192522 |
| 3893 | 1753 | TTCATTTGTGAAATTTGTGGGGCACAATTTactcagaaaggtaacctcctccgccacattaaaCTG | 46F_47I_49E_50I_52G_53A_54Q_55F_67L_ | 2.862032448 |
| 3894 | 1900 | TTCATATGTCCCGTATGTGGGGCAGCCTTTactcagaaaggtaacctcctccgccacattaaaCTG | 46F_47I_49P_50V_52G_53A_54A_55F_67L_ | 2.861974871 |
| 3895 | 4557 | TTCTATTGTCCAGTTTGTGGGGCAGTGTTTactcagaaaggtaacctcctccgccacattaaaCTG | 46F_47Y_49P_50V_52G_53A_54V_55F_67L_ | 2.858844518 |
| 3896 | 3612 | TTCACTTGTGATATCTGTGGGGCACCCTTTactcagaaaggtaacctcctccgccacattaaaCTG | 46F_47T_49D_50I_52G_53A_54P_55F_67L_ | 2.855658351 |
| 3897 | 1873 | TTCATTTGTCCAATTTGTGGGGCACAGTTTactcagaaaggtaacctcctccgccacattaaaCTG | 46F_47I_49P_50I_52G_53A_54Q_55F_67L_ | 2.855508739 |
| 3898 | 1452 | TTCCATTGTGACATCTGTGGGGCACCGTTTactcagaaaggtaacctcctccgccacattaaaCTG | 46F_47H_49D_50I_52G_53A_54P_55F_67L_ | 2.853847411 |
| 3899 | 355 | TTCTGTTGTGAGGTCTGTGGGGCAAGTTTTactcagaaaggtaacctcctccgccacattaaaCTG | 46F_47C_49E_50V_52G_53A_54S_55F_67L_ | 2.852232419 |
| 3900 | 4020 | TTCGTGTGTCCGATCTGTGGGGCAGCGTTTactcagaaaggtaacctcctccgccacattaaaCTG | 46F_47V_49P_50I_52G_53A_54A_55F_67L_ | 2.850280091 |
| 3901 | 4540 | TTCTACTGTCCAGTCTGTGGGGCAGCTTTTactcagaaaggtaacctcctccgccacattaaaCTG | 46F_47Y_49P_50V_52G_53A_54A_55F_67L_ | 2.849926102 |
| 3902 | 4240 | TTCTGGTGTAATGTCTGTGGGGCAGCCTTTactcagaaaggtaacctcctccgccacattaaaCTG | 46F_47W_49N_50V_52G_53A_54A_55F_67L_ | 2.847491766 |
| 3903 | 1192 | TTCTTTTGTCCAGTGTGTGGGGCACCTTTTactcagaaaggtaacctcctccgccacattaaaCTG | 46F_47F_49P_50V_52G_53A_54P_55F_67L_ | 2.843362615 |
| 3904 | 4446 | TTCTACTGTAACATATGTGGGGCACATTTTactcagaaaggtaacctcctccgccacattaaaCTG | 46F_47Y_49N_50I_52G_53A_54H_55F_67L_ | 2.843316542 |
| 3905 | 420 | TTCTGCTGTCCTATCTGTGGGGCAGCCTTTactcagaaaggtaacctcctccgccacattaaaCTG | 46F_47C_49P_50I_52G_53A_54A_55F_67L_ | 2.843014982 |
| 3906 | 1039 | TTCTTTTGTGAAATATGTGGGGCATATTTTactcagaaaggtaacctcctccgccacattaaaCTG | 46F_47F_49E_50I_52G_53A_54Y_55F_67L_ | 2.842195671 |
| 3907 | 1143 | TTCTTCTGTCCAATTTGTGGGGCAGAGTTTactcagaaaggtaacctcctccgccacattaaaCTG | 46F_47F_49P_50I_52G_53A_54E_55F_67L_ | 2.839726056 |
| 3908 | 1186 | TTCTTTTGTCCTGTCTGTGGGGCACATTTTactcagaaaggtaacctcctccgccacattaaaCTG | 46F_47F_49P_50V_52G_53A_54H_55F_67L_ | 2.836642113 |
| 3909 | 6756 | CTCAGCTGTCCTATTTGTGGATTTACCTGTactcagaaaggtaacctcctccgccacattaaaCTG | 46L_47S_49P_50I_52G_53F_54T_55C_67L_ | 2.833850008 |
| 3910 | 4075 | TTCGTCTGTCCTGTGTGTGGGGCATCTTTTactcagaaaggtaacctcctccgccacattaaaCTG | 46F_47V_49P_50V_52G_53A_54S_55F_67L_ | 2.83289034 |
| 3911 | 1146 | TTCTTCTGTCCTATCTGTGGGGCACACTTTactcagaaaggtaacctcctccgccacattaaaCTG | 46F_47F_49P_50I_52G_53A_54H_55F_67L_ | 2.831449812 |
| 3912 | 1911 | TTCATCTGTCCAGTGTGTGGGGCAAATTTTactcagaaaggtaacctcctccgccacattaaaCTG | 46F_47I_49P_50V_52G_53A_54N_55F_67L_ | 2.827685833 |
| 3913 | 4507 | TTCTATTGTCCTATATGTGGGGCAATTTTTactcagaaaggtaacctcctccgccacattaaaCTG | 46F_47Y_49P_50I_52G_53A_54I_55F_67L_ | 2.824936063 |
| 3914 | 4548 | TTCTACTGTCCCGTCTGTGGGGCAAAATTTactcagaaaggtaacctcctccgccacattaaaCTG | 46F_47Y_49P_50V_52G_53A_54K_55F_67L_ | 2.823446788 |
| 3915 | 4376 | TTCTACTGTGACGTTTGTGGGGCAACCTTTactcagaaaggtaacctcctccgccacattaaaCTG | 46F_47Y_49D_50V_52G_53A_54T_55F_67L_ | 2.822897604 |
| 3916 | 2232 | TTCCTGTGTGAAATCTGTGGGGCACCATTTactcagaaaggtaacctcctccgccacattaaaCTG | 46F_47L_49E_50I_52G_53A_54P_55F_67L_ | 2.821787019 |
| 3917 | 1381 | TTCGGCTGTCCCATTTGTGGGGCATGTTTTactcagaaaggtaacctcctccgccacattaaaCTG | 46F_47G_49P_50I_52G_53A_54C_55F_67L_ | 2.820068909 |
| 3918 | 6599 | CTTCATTGCCCCATCTGCGGGTTCTACTGCactcagaaaggtaacctcctccgccacattaaaCTG | 46L_47H_49P_50I_52G_53F_54Y_55C_67L_ | 2.815950193 |
| 3919 | 6824 | CTTTACTGTCCCATATGCGGGTTTTTCTGTactcagaaaggtaacctcctccgccacattaaaCTG | 46L_47Y_49P_50I_52G_53F_54F_55C_67L_ | 2.814632615 |
| 3920 | 1093 | TTCTTCTGTAACATTTGTGGGGCACAATTTactcagaaaggtaacctcctccgccacattaaaCTG | 46F_47F_49N_50I_52G_53A_54Q_55F_67L_ | 2.812311012 |
| 3921 | 3435 | TTCTCGTGTGAGATCTGTGGGGCATCGTTTactcagaaaggtaacctcctccgccacattaaaCTG | 46F_47S_49E_50I_52G_53A_545_55F_67L_ | 2.803051461 |
| 3922 | 4171 | TTCTGGTGTGAGCAGTGTGGGGCAAACTTTactcagaaaggtaacctcctccgccacattaaaCTG | 46F_47W_49E_50Q_52G_53A_54N_55F_67L_ | 2.802983498 |
| 3923 | 75 | TTCGCCTGTGAGATCTGTGGGGCATCATTTactcagaaaggtaacctcctccgccacattaaaCTG | 46F_47A_49E_50I_52G_53A_54S_55F_67L_ | 2.801628035 |
| 3924 | 3475 | TTCAGCTGTGAAGTGTGTGGGGCAAGTTTTactcagaaaggtaacctcctccgccacattaaaCTG | 46F_47S_49E_50V_52G_53A_54S_55F_67L_ | 2.798778218 |
| 3925 | 2955 | TTCCAATGTGAGATCTGTGGGGCAAGCTTTactcagaaaggtaacctcctccgccacattaaaCTG | 46F_47Q_49E_50I_52G_53A_54S_55F_67L_ | 2.798572139 |
| 3926 | 1633 | TTCCACTGTCCGATCTGTGGGGCACAGTTTactcagaaaggtaacctcctccgccacattaaaCTG | 46F_47H_49P_50I_52G_53A_54Q_55F_67L_ | 2.79672151 |
| 3927 | 1793 | TTCATTTGTGAGGTATGTGGGGCACAGTTTactcagaaaggtaacctcctccgccacattaaaCTG | 46F_47I_49E_50V_52G_53A_54Q_55F_67L_ | 2.796186039 |
| 3928 | 6479 | CTTGCATGCCCGATCTGCGGCTTTTACTGTactcagaaaggtaacctcctccgccacattaaaCTG | 46L_47A_49P_50I_52G_53F_54Y_55C_67L_ | 2.793600846 |
| 3929 | 7614 | CTCGCTTGTGAAATTTGTGGTTTCAGATGTactcagaaaggtaacctcctccgccacattaaaCTG | 46L_47A_49E_50I_52G_53F_54R_55C_67L_ | 2.791630581 |
| 3930 | 4185 | TTCTGGTGTGAAGTGTGTGGGGCAGGATTTactcagaaaggtaacctcctccgccacattaaaCTG | 46F_47W_49E_50V_52G_53A_54G_55F_67L_ | 2.789362587 |
| 3931 | 2940 | TTCCAATGTGAAATCTGTGGGGCAGCATTTactcagaaaggtaacctcctccgccacattaaaCTG | 46F_47Q_49E_50I_52G_53A_54A_55F_67L_ | 2.785685145 |
| 3932 | 3954 | TTCGTATGTGAGGTCTGTGGGGCACGATTTactcagaaaggtaacctcctccgccacattaaaCTG | 46F_47V_49E_50V_52G_53A_54R_55F_67L_ | 2.783776213 |
| 3933 | 4424 | TTCTATTGTGAGGTCTGTGGGGCATTCTTTactcagaaaggtaacctcctccgccacattaaaCTG | 46F_47Y_49E_50V_52G_53A_54F_55F_67L_ | 2.783182981 |
| 3934 | 4143 | TTCTGGTGTGAGATTTGTGGGGCAGAGTTTactcagaaaggtaacctcctccgccacattaaaCTG | 46F_47W_49E_50I_52G_53A_54E_55F_67L_ | 2.781222662 |
| 3935 | 1024 | TTCTTTTGTGAAATTTGTGGGGCATTCTTTactcagaaaggtaacctcctccgccacattaaaCTG | 46F_47F_49E_50I_52G_53A_54F_55F_67L_ | 2.78060629 |
| 3936 | 6675 | CTAATGTGTCCAATTTGCGGTTTCAGCTGTactcagaaaggtaacctcctccgccacattaaaCTG | 46L_47M_49P_50I_52G_53F_54S_55C_67L_ | 2.780550004 |
| 3937 | 4035 | TTCGTCTGTCCTATCTGTGGGGCAAGTTTTactcagaaaggtaacctcctccgccacattaaaCTG | 46F_47V_49P_50I_52G_53A_54S_55F_67L_ | 2.780424004 |
| 3938 | 1620 | TTCCATTGTCCAATATGTGGGGCAGCCTTTactcagaaaggtaacctcctccgccacattaaaCTG | 46F_47H_49P_50I_52G_53A_54A_55F_67L_ | 2.78022868 |
| 3939 | 3555 | TTCAGTIGTCCTATCTGTGGGGCAAGCTTTactcagaaaggtaacctcctccgccacattaaaCTG | 46F_47S_49P_50I_52G_53A_54S_55F_67L_ | 2.780010063 |
| 3940 | 4295 | TTCTGGTGTCCTCAATGTGGGGCAAGTTTTactcagaaaggtaacctcctccgccacattaaaCTG | 46F_47W_49P_50Q_52G_53A_54S_55F_67L_ | 2.778454492 |
| 3941 | 7636 | CTTTGCTGTGAGATCTGTGGTTTTACATGTactcagaaaggtaacctcctccgccacattaaaCTG | 46L_47C_49E_50I_52G_53F_54T_55C_67L_ | 2.777075335 |
| 3942 | 6550 | CTTTTCTGCCCAATCTGCGGATTCATGTGTactcagaaaggtaacctcctccgccacattaaaCTG | 46L_47F_49P_50I_52G_53F_54M_55C_67L_ | 2.77251114 |
| 3943 | 976 | TTCTTCTGTGACATTTGTGGGGCAACCTTTactcagaaaggtaacctcctccgccacattaaaCTG | 46F_47F_49D_50I_52G_53A_54T_55F_67L_ | 2.769382701 |
| 3944 | 3101 | TTCCAATGTCCTGTCTGTGGGGCATGCTTTactcagaaaggtaacctcctccgccacattaaaCTG | 46F_47Q_49P_50V_52G_53A_54C_55F_67L_ | 2.7681274 |
| 3945 | 4175 | TTCTGGTGTGAGCAGTGTGGGGCATCCTTTactcagaaaggtaacctcctccgccacattaaaCTG | 46F_47W_49E_50Q_52G_53A_54S_55F_67L_ | 2.766631729 |
| 3946 | 4270 | TTCTGGTGTCCAATTTGTGGGGCAATGTTTactcagaaaggtaacctcctccgccacattaaaCTG | 46F_47W_49P_50I_52G_53A_54M_55F_67L_ | 2.765114199 |
| 3947 | 7894 | TTAAGCTGCGAGATTTGTGGCTTTAGATGCactcagaaaggtaacctcctccgccacattaaaCTG | 46L_47S_49E_50I_52G_53F_54R_55C_67L_ | 2.760025066 |
| 3948 | 4216 | TTCTGGTGTAATATCTGTGGGGCAACATTTactcagaaaggtaacctcctccgccacattaaaCTG | 46F_47W_49N_50I_52G_53A_54T_55F_67L_ | 2.757413832 |
| 3949 | 6715 | TTACAGTGTCCAATCTGCGGATTTAGCTGTactcagaaaggtaacctcctccgccacattaaaCTG | 46L_47Q_49P_50I_52G_53F_54S_55C_67L_ | 2.75725052 |
| 3950 | 4366 | TTCTATTGTGATGTTTGTGGGGCACATTTTactcagaaaggtaacctcctccgccacattaaaCTG | 46F_47Y_49D_50V_52G_53A_54H_55F_67L_ | 2.756831872 |
| 3951 | 2997 | TTCCAGTGTGAGGTATGTGGGGCAGTGTTTactcagaaaggtaacctcctccgccacattaaaCTG | 46F_47Q_49E_50V_52G_53A_54V_55F_67L_ | 2.756470588 |
| 3952 | 1751 | TTCATCTGTGAAATCTGTGGGGCAAATTTTactcagaaaggtaacctcctccgccacattaaaCTG | 46F_47I_49E_50I_52G_53A_54N_55F_67L_ | 2.752719692 |
| 3953 | 4217 | TTCTGGTGTAATATCTGTGGGGCAGTCTTTactcagaaaggtaacctcctcogccacattaaaCTG | 46F_47W_49N_50I_52G_53A_54V_55F_67L_ | 2.750109354 |
| 3954 | 7568 | TTCTGGTGCGAGATCTGTGGCCGCAAGTTTactcagaaaggtaacctcctccgccacattaaaCTG | 46F_47W_49E_50I_52G_53R_54K_55F_67L_ | 2.749639875 |
| 3955 | 7971 | CTCTACTGTGAAATTTGTGGATTCAATTGTactcagaaaggtaacctcctccgccacattaaaCTG | 46L_47Y_49E_50I_52G_53F_54N_55C_67L_ | 2.748701854 |
| 3956 | 1097 | TTCTTTTGTAACATTTGTGGGGCAGTTTTTactcagaaaggtaacctcctccgccacattaaaCTG | 46F_47F_49N_50I_52G_53A_54V_55F_67L_ | 2.746961967 |
| 3957 | 434 | TTCTGCTGTCCCATTTGTGGGGCACGCTTTactcagaaaggtaacctcctccgccacattaaaCTG | 46F_47C_49P_50I_52G_53A_54R_55F_67L_ | 2.746495917 |
| 3958 | 6545 | CTCTTCTGCCCAATCTGTGGCTTTGGATGTactcagaaaggtaacctcctccgccacattaaaCTG | 46L_47F_49P_50I_52G_53F_54G_55C_67L_ | 2.742897533 |
| 3959 | 3076 | TTCCAGTGTCCAATATGTGGGGCAACCTTTactcagaaaggtaacctcctccgccacattaaaCTG | 46F_47Q_49P_50I_52G_53A_54T_55F_67L_ | 2.742415436 |
| 3960 | 1871 | TTCATCTGTCCTATCTGTGGGGCAAACTTTactcagaaaggtaacctcctccgccacattaaaCTG | 46F_47I_49P_50I_52G_53A_54N_55F_67L_ | 2.74212871 |
| 3961 | 3953 | TTCGTTTGTGAAGTGTGTGGGGCACAATTTactcagaaaggtaacctcctccgccacattaaaCTG | 46F_47V_49E_50V_52G_53A_54Q_55F_67L_ | 2.741771238 |
| 3962 | 1913 | TTCATCTGTCCTGTGTGTGGGGCACAATTTactcagaaaggtaacctcctccgccacattaaaCTG | 46F_47I_49P_50V_52G_53A_54Q_55F_67L_ | 2.739300663 |
| 3963 | 4087 | TTCTGGTGTGACATTTGTGGGGCAATCTTTactcagaaaggtaacctcctccgccacattaaaCTG | 46F_47W_49D_50I_52G_53A_54I_55F_67L_ | 2.738252031 |
| 3964 | 1314 | TTCGGGTGTGAGGTGTGTGGGGCACGATTTactcagaaaggtaacctcctccgccacattaaaCTG | 46F_47G_49E_50V_52G_53A_54R_55F_67L_ | 2.73673792 |
| 3965 | 1915 | TTCATCTGTCCAGTGTGTGGGGCATCCTTTactcagaaaggtaacctcctccgccacattaaaCTG | 46F_47I_49P_50V_52G_53A_54S_55F_67L_ | 2.735974298 |
| 3966 | 372 | TTCTGTTGTAACATATGTGGGGCACCTTTTactcagaaaggtaacctcctccgccacattaaaCTG | 46F_47C_49N_50I_52G_53A_54P_55F_67L_ | 2.73273416 |
| 3967 | 4510 | TTCTACTGTCCCATTTGTGGGGCAATGTTTactcagaaaggtaacctcctccgccacattaaaCTG | 46F_47Y_49P_50I_52G_53A_54M_55F_67L_ | 2.732409041 |
| 3968 | 115 | TTCGCTTGTGAGGTGTGTGGGGCAAGTTTTactcagaaaggtaacctcctccgccacattaaaCTG | 46F_47A_49E_50V_52G_53A_54S_55F_67L_ | 2.732100307 |
| 3969 | 4265 | TTCTGGTGTCCAATCTGTGGGGCAGGTTTTactcagaaaggtaacctcctccgccacattaaaCTG | 46F_47W_49P_50I_52G_53A_54G_55F_67L_ | 2.728973918 |
| 3970 | 1755 | TTCATATGTGAGATTTGTGGGGCATCTTTTactcagaaaggtaacctcctccgccacattaaaCTG | 46F_47I_49E_50I_52G_53A_54S_55F_67L_ | 2.728055745 |
| 3971 | 1015 | TTCTTTTGTGATGTGTGTGGGGCATCCTTTactcagaaaggtaacctcctccgccacattaaaCTG | 46F_47F_49D_50V_52G_53A_54S_55F_67L_ | 2.727593397 |
| 3972 | 6559 | CTGTTTTGTCCAATCTGCGGGTTCTATTGCactcagaaaggtaacctcctccgccacattaaaCTG | 46L_47F_49P_50I_52G_53F_54Y_55C_67L_ | 2.725378374 |
| 3973 | 6547 | CTGTTCTGTCCAATATGTGGCTTCATCTGTactcagaaaggtaacctcctccgccacattaaaCTG | 46L_47F_49P_50I_52G_53F_54I_55C_67L_ | 2.723694714 |
| 3974 | 3835 | TTCACCTGTCCAGTTTGTGGGGCAAGCTTTactcagaaaggtaacctcctccgccacattaaaCTG | 46F_47T_49P_50V_52G_53A_54S_55F_67L_ | 2.722212595 |
| 3975 | 4307 | TTCTGGTGTCCCGTTTGTGGGGCAATTTTTactcagaaaggtaacctcctccgccacattaaaCTG | 46F_47W_49P_50V_52G_53A_54I_55F_67L_ | 2.720391292 |
| 3976 | 7699 | TTATTCTGCGAAATCTGCGGTTTTTATTGCactcagaaaggtaacctcctccgccacattaaaCTG | 46L_47F_49E_50I_52G_53F_54Y_55C_67L_ | 2.719658405 |
| 3977 | 1267 | TTCGGCTGTGAGATTTGTGGGGCAATTTTTactcagaaaggtaacctcctccgccacattaaaCTG | 46F_47G_49E_50I_52G_53A_54I_55F_67L_ | 2.71928121 |
| 3978 | 3115 | TTCCAATGTCCTGTGTGTGGGGCATCATTTactcagaaaggtaacctcctccgccacattaaaCTG | 46F_47Q_49P_50V_52G_53A_54S_55F_67L_ | 2.718295342 |
| 3979 | 3073 | TTCCAGTGTCCAATCTGTGGGGCACAGTTTactcagaaaggtaacctcctccgccacattaaaCTG | 46F_47Q_49P_50I_52G_53A_54Q_55F_67L_ | 2.717781747 |
| 3980 | 6793 | CTGGTCTGCCCTATCTGCGGGTTTCAGTGCactcagaaaggtaacctcctccgccacattaaaCTG | 46L_47V_49P_50I_52G_53F_54Q_55C_67L_ | 2.717397005 |
| 3981 | 1065 | TTCTTTTGTGAGGTGTGTGGGGCAGGATTTactcagaaaggtaacctcctccgccacattaaaCTG | 46F_47F_49E_50V_52G_53A_54G_55F_67L_ | 2.716322652 |
| 3982 | 1197 | TTCTTTTGTCCCGTCTGTGGGGCAGTCTTTactcagaaaggtaacctcctccgccacattaaaCTG | 46F_47F_49P_50V_52G_53A_54V_55F_67L_ | 2.716308468 |
| 3983 | 6496 | TTGTGCTGTCCGATATGTGGATTCACCTGTactcagaaaggtaacctcctccgccacattaaaCTG | 46L_47C_49P_50I_52G_53F_54T_55C_67L_ | 2.712956048 |
| 3984 | 435 | TTCTGTTGTCCAATTTGTGGGGCATCCTTTactcagaaaggtaacctcctccgccacattaaaCTG | 46F_47C_49P_50I_52G_53A_54S_55F_67L_ | 2.710360655 |
| 3985 | 6581 | CTGCACTGCCCAATTTGCGGGTTCTGCTGTactcagaaaggtaacctcctccgccacattaaaCTG | 46L_47H_49P_50I_52G_53F_54C_55C_67L_ | 2.710108007 |
| 3986 | 2957 | TTCCAGTGTGAAATTTGTGGGGCAGTTTTTactcagaaaggtaacctcctccgccacattaaaCTG | 46F_47Q_49E_50I_52G_53A_54V_55F_67L_ | 2.708168388 |
| 3987 | 6579 | CTCGGGTGCCCAATCTGCGGCTTCTACTGCactcagaaaggtaacctcctccgccacattaaaCTG | 46L_47G_49P_50I_52G_53F_54Y_55C_67L_ | 2.70448374 |
| 3988 | 1011 | TTCTTTTGTGATGTTTGTGGGGCAAACTTTactcagaaaggtaacctcctccgccacattaaaCTG | 46F_47F_49D_50V_52G_53A_54N_55F_67L_ | 2.703672097 |
| 3989 | 315 | TTCTGCTGTGAGATCTGTGGGGCATCATTTactcagaaaggtaacctcctccgccacattaaaCTG | 46F_47C_49E_50I_52G_53A_54S_55F_67L_ | 2.702913686 |
| 3990 | 7920 | TTAGTGTGCGAGATTTGTGGGTTCGCCTGCactcagaaaggtaacctcctccgccacattaaaCTG | 46L_47V_49E_50I_52G_53F_54A_55C_67L_ | 2.701871352 |
| 3991 | 6474 | CTAGCCTGTCCAATATGCGGCTTCCGCTGTactcagaaaggtaacctcctccgccacattaaaCTG | 46L_47A_49P_50I_52G_53F_54R_55C_67L_ | 2.700499172 |
| 3992 | 6807 | CTTTGGTGTCCTATATGCGGCTTTATTTGCactcagaaaggtaacctcctccgccacattaaaCTG | 46L_47W_49P_50I_52G_53F_54I_55C_67L_ | 2.695357625 |
| 3993 | 7574 | TTCTGGTGCGAAATCTGTGGGCGGAGATTTactcagaaaggtaacctcctccgccacattaaaCTG | 46F_47W_49E_50I_52G_53R_54R_55F_67L_ | 2.694636817 |
| 3994 | 6544 | CTGTTCTGCCCCATCTGCGGATTCTTTTGTactcagaaaggtaacctcctccgccacattaaaCTG | 46L_47F_49P_50I_52G_53F_54F_55C_67L_ | 2.694019049 |
| 3995 | 6600 | CTAATCTGTCCAATCTGCGGGTTTGCATGCactcagaaaggtaacctcctccgccacattaaaCTG | 46L_47I_49P_50I_52G_53F_54A_55C_67L_ | 2.692485025 |
| 3996 | 4279 | TTCTGGTGTCCTATTTGTGGGGCATATTTTactcagaaaggtaacctcctccgccacattaaaCTG | 46F_47W_49P_50I_52G_53A_54Y_55F_67L_ | 2.690933358 |
| 3997 | 4189 | TTCTGGTGTGAAGTTTGTGGGGCACTCTTTactcagaaaggtaacctcctccgccacattaaaCTG | 46F_47W_49E_50V_52G_53A_54L_55F_67L_ | 2.690243653 |
| 3998 | 3432 | TTCTCCTGTGAGATTTGTGGGGCACCCTTTactcagaaaggtaacctcctccgccacattaaaCTG | 46F_47S_49E_50I_52G_53A_54P_55F_67L_ | 2.68797007 |
| 3999 | 3540 | TTCAGCTGTCCAATATGTGGGGCAGCTTTTactcagaaaggtaacctcctccgccacattaaaCTG | 46F_47S_49P_50I_52G_53A_54A_55F_67L_ | 2.687920948 |
| 4000 | 2981 | TTCCAGTGTGAAGTGTGTGGGGCATGCTTTactcagaaaggtaacctcctccgccacattaaaCTG | 46F_47Q_49E_50V_52G_53A_54C_55F_67L_ | 2.68731385 |
| 4001 | 1081 | TTCTTCTGTAATATTTGTGGGGCATGCTTTactcagaaaggtaacctcctccgccacattaaaCTG | 46F_47F_49N_50I_52G_53A_54C_55F_67L_ | 2.686211158 |
| 4002 | 6480 | CTGTGTTGCCCCATCTGTGGCTTTGCATGCactcagaaaggtaacctcctccgccacattaaaCTG | 46L_47C_49P_50I_52G_53F_54A_55C_67L_ | 2.684663616 |
| 4003 | 1080 | TTCTTCTGTAATATCTGTGGGGCAGCATTTactcagaaaggtaacctcctccgccacattaaaCTG | 46F_47F_49N_50I_52G_53A_54A_55F_67L_ | 2.679579115 |
| 4004 | 4544 | TTCTACTGTCCCGTCTGTGGGGCATTCTTTactcagaaaggtaacctcctccgccacattaaaCTG | 46F_47Y_49P_50V_52G_53A_54F_55F_67L_ | 2.678644017 |
| 4005 | 6759 | CTGTCTTGTCCTATCTGTGGTTTCTATTGCactcagaaaggtaacctcctccgccacattaaaCTG | 46L_47S_49P_50I_52G_53F_54Y_55C_67L_ | 2.677293087 |
| 4006 | 1515 | TTCCACTGTGAGATTTGTGGGGCATCTTTTactcagaaaggtaacctcctccgccacattaaaCTG | 46F_47H_49E_50I_52G_53A_54S_55F_67L_ | 2.674877795 |
| 4007 | 4503 | TTCTACTGTCCGATTTGTGGGGCAGAATTTactcagaaaggtaacctcctccgccacattaaaCTG | 46F_47Y_49P_50I_52G_53A_54E_55F_67L_ | 2.674845423 |
| 4008 | 6593 | TTGCACTGCCCTATCTGTGGATTTCAATGCactcagaaaggtaacctcctccgccacattaaaCTG | 46L_47H_49P_50I_52G_53F_54Q_55C_67L_ | 2.673631529 |
| 4009 | 6761 | TTAACTTGCCCCATTTGTGGCTTCTGCTGCactcagaaaggtaacctcctccgccacattaaaCTG | 46L_47T_49P_50I_52G_53F_54C_55C_67L_ | 2.672412042 |
| 4010 | 4456 | TTCTATTGTAACATCTGTGGGGCAACATTTactcagaaaggtaacctcctccgccacattaaaCTG | 46F_47Y_49N_50I_52G_53A_54T_55F_67L_ | 2.672380179 |
| 4011 | 2392 | TTCCTGTGTCCCGTCTGTGGGGCACCTTTTactcagaaaggtaacctcctccgccacattaaaCTG | 46F_47L_49P_50V_52G_53A_54P_55F_67L_ | 2.671186483 |
| 4012 | 6494 | CTGTGCTGTCCTATATGCGGATTTAGGTGCactcagaaaggtaacctcctccgccacattaaaCTG | 46L_47C_49P_50I_52G_53F_54R_55C_67L_ | 2.667800276 |
| 4013 | 1183 | TTCTTCTGTCCGGTGTGTGGGGCAGAATTTactcagaaaggtaacctcctccgccacattaaaCTG | 46F_47F_49P_50V_52G_53A_54E_55F_67L_ | 2.667653763 |
| 4014 | 1865 | TTCATCTGTCCCATCTGTGGGGCAGGCTTTactcagaaaggtaacctcctccgccacattaaaCTG | 46F_47I_49P_50I_52G_53A_54G_55F_67L_ | 2.66692475 |
| 4015 | 4065 | TTCGTGTGTCCCGTCTGTGGGGCAGGATTTactcagaaaggtaacctcctccgccacattaaaCTG | 46F_47V_49P_50V_52G_53A_54G_55F_67L_ | 2.66507977 |
| 4016 | 1785 | TTCATCTGTGAGGTGTGTGGGGCAGGCTTTactcagaaaggtaacctcctccgccacattaaaCTG | 46F_47I_49E_50V_52G_53A_54G_55F_67L_ | 2.664022574 |
| 4017 | 977 | TTCTTTTGTGACATCTGTGGGGCAGTGTTTactcagaaaggtaacctcctccgccacattaaaCTG | 46F_47F_49D_50I_52G_53A_54V_55F_67L_ | 2.662070804 |
| 4018 | 4368 | TTCTATTGTGACGTATGTGGGGCAAAATTTactcagaaaggtaacctcctccgccacattaaaCTG | 46F_47Y_49D_50V_52G_53A_54K_55F_67L_ | 2.660405862 |
| 4019 | 4212 | TTCTGGTGTAATATCTGTGGGGCACCATTTactcagaaaggtaacctcctccgccacattaaaCTG | 46F_47W_49N_50I_52G_53A_54P_55F_67L_ | 2.658932724 |
| 4020 | 1754 | TTCATCTGTGAGATTTGTGGGGCACGTTTTactcagaaaggtaacctcctccgccacattaaaCTG | 46F_47I_49E_50I_52G_53A_54R_55F_67L_ | 2.656112861 |
| 4021 | 433 | TTCTGCTGTCCTATTTGTGGGGCACAATTTactcagaaaggtaacctcctccgccacattaaaCTG | 46F_47C_49P_50I_52G_53A_54Q_55F_67L_ | 2.655744427 |
| 4022 | 1433 | TTCGGGTGTCCAGTTTGTGGGGCACAGTTTactcagaaaggtaacctcctccgccacattaaaCTG | 46F_47G_49P_50V_52G_53A_54Q_55F_67L_ | 2.652607644 |
| 4023 | 1393 | TTCGGCTGTCCAATTTGTGGGGCACAGTTTactcagaaaggtaacctcctccgccacattaaaCTG | 46F_47G_49P_50I_52G_53A_54Q_55F_67L_ | 2.647943989 |
| 4024 | 1273 | TTCGGCTGTGAGATATGTGGGGCACAGTTTactcagaaaggtaacctcctccgccacattaaaCTG | 46F_47G_49E_50I_52G_53A_54Q_55F_67L_ | 2.643829277 |
| 4025 | 1188 | TTCTTCTGTCCCGTGTGTGGGGCAAAGTTTactcagaaaggtaacctcctccgccacattaaaCTG | 46F_47F_49P_50V_52G_53A_54K_55F_67L_ | 2.64364492 |
| 4026 | 2996 | TTCCAGTGTGAGGTCTGTGGGGCAACGTTTactcagaaaggtaacctcctccgccacattaaaCTG | 46F_47Q_49E_50V_52G_53A_54T_55F_67L_ | 2.643059949 |
| 4027 | 1621 | TTCCACTGTCCTATTTGTGGGGCATGCTTTactcagaaaggtaacctcctccgccacattaaaCTG | 46F_47H_49P_50I_52G_53A_54C_55F_67L_ | 2.642924034 |
| 4028 | 4267 | TTCTGGTGTCCCATTTGTGGGGCAATCTTTactcagaaaggtaacctcctccgccacattaaaCTG | 46F_47W_49P_50I_52G_53A_54I_55F_67L_ | 2.642807279 |
| 4029 | 1555 | TTCCACTGTGAGGTGTGTGGGGCATCTTTTactcagaaaggtaacctcctccgccacattaaaCTG | 46F_47H_49E_50V_52G_53A_54S_55F_67L_ | 2.639451258 |
| 4030 | 1513 | TTCCATTGTGAGATCTGTGGGGCACAATTTactcagaaaggtaacctcctccgccacattaaaCTG | 46F_47H_49E_50I_52G_53A_54Q_55F_67L_ | 2.63941003 |
| 4031 | 120 | TTCGCCTGTAACATTTGTGGGGCAGCATTTactcagaaaggtaacctcctccgccacattaaaCTG | 46F_47A_49N_50I_52G_53A_54A_55F_67L_ | 2.637959402 |
| 4032 | 6803 | TTGTGGTGTCCTATTTGTGGGTTCGAGTGCactcagaaaggtaacctcctccgccacattaaaCTG | 46L_47W_49P_50I_52G_53F_54E_55C_67L_ | 2.637165571 |
| 4033 | 4559 | TTCTACTGTCCTGTTTGTGGGGCATACTTTactcagaaaggtaacctcctccgccacattaaaCTG | 46F_47Y_49P_50V_52G_53A_54Y_55F_67L_ | 2.636185029 |
| 4034 | 3833 | TTCACTTGTCCTGTGTGTGGGGCACAGTTTactcagaaaggtaacctcctccgccacattaaaCTG | 46F_47T_49P_50V_52G_53A_54Q_55F_67L_ | 2.634905136 |
| 4035 | 3791 | TTCACATGTCCTATATGTGGGGCAAATTTTactcagaaaggtaacctcctccgccacattaaaCTG | 46F_47T_49P_50I_52G_53A_54N_55F_67L_ | 2.631764831 |
| 4036 | 6580 | CTGCACTGCCCAATCTGTGGATTCGCGTGCactcagaaaggtaacctcctccgccacattaaaCTG | 46L_47H_49P_50I_52G_53F_54A_55C_67L_ | 2.628100624 |
| 4037 | 4031 | TTCGTCTGTCCAATCTGTGGGGCAAACTTTactcagaaaggtaacctcctccgccacattaaaCTG | 46F_47V_49P_50I_52G_53A_54N_55F_67L_ | 2.62759808 |
| 4038 | 1069 | TTCTTTTGTGAGGTGTGTGGGGCACTGTTTactcagaaaggtaacctcctccgccacattaaaCTG | 46F_47F_49E_50V_52G_53A_54L_55F_67L_ | 2.626098971 |
| 4039 | 3600 | TTCACATGTGATATCTGTGGGGCAGCCTTTactcagaaaggtaacctcctccgccacattaaaCTG | 46F_47T_49D_50I_52G_53A_54A_55F_67L_ | 2.62485359 |
| 4040 | 3220 | TTCCGATGTGAAGTGTGTGGGGCAGCATTTactcagaaaggtaacctcctccgccacattaaaCTG | 46F_47R_49E_50V_52G_53A_54A_55F_67L_ | 2.620888584 |
| 4041 | 4073 | TTCGTCTGTCCTGTGTGTGGGGCACAGTTTactcagaaaggtaacctcctccgccacattaaaCTG | 46F_47V_49P_50V_52G_53A_54Q_55F_67L_ | 2.620315856 |
| 4042 | 6575 | CTTGGCTGCCCAATTTGCGGGTTCTCTTGTactcagaaaggtaacctcctccgccacattaaaCTG | 46L_47G_49P_50I_52G_53F_54S_55C_67L_ | 2.617817848 |
| 4043 | 2872 | TTCAATTGTCCAGTGTGTGGGGCACCCTTTactcagaaaggtaacctcctccgccacattaaaCTG | 46F_47N_49P_50V_52G_53A_54P_55F_67L_ | 2.617409954 |
| 4044 | 3781 | TTCACGTGTCCGATTTGTGGGGCATGTTTTactcagaaaggtaacctcctccgccacattaaaCTG | 46F_47T_49P_50I_52G_53A_54C_55F_67L_ | 2.616898984 |
| 4045 | 1380 | TTCGGGTGTCCCATATGTGGGGCAGCTTTTactcagaaaggtaacctcctccgccacattaaaCTG | 46F_47G_49P_50I_52G_53A_54A_55F_67L_ | 2.614644725 |
| 4046 | 6796 | TTAGTCTGCCCTATCTGCGGTTTTACCTGCactcagaaaggtaacctcctccgccacattaaaCTG | 46L_47V_49P_50I_52G_53F_54T_55C_67L_ | 2.610859708 |
| 4047 | 6774 | CTCACCTGCCCTATATGCGGTTTTCGCTGCactcagaaaggtaacctcctccgccacattaaaCTG | 46L_47T_49P_50I_52G_53F_54R_55C_67L_ | 2.610122443 |
| 4048 | 7947 | TTGTGGTGTGAAATCTGCGGCTTCATTTGCactcagaaaggtaacctcctccgccacattaaaCTG | 46L_47W_49E_50I_52G_53F_54I_55C_67L_ | 2.609455086 |
| 4049 | 1091 | TTCTTTTGTAATATTTGTGGGGCAAATTTTactcagaaaggtaacctcctccgccacattaaaCTG | 46F_47F_49N_50I_52G_53A_54N_55F_67L_ | 2.606101124 |
| 4050 | 3951 | TTCGTGTGTGAAGTGTGTGGGGCAAACTTTactcagaaaggtaacctcctccgccacattaaaCTG | 46F_47V_49E_50V_52G_53A_54N_55F_67L_ | 2.603619661 |
| 4051 | 1553 | TTCCATTGTGAGGTATGTGGGGCACAGTTTactcagaaaggtaacctcctccgccacattaaaCTG | 46F_47H_49E_50V_52G_53A_54Q_55F_67L_ | 2.603213005 |
| 4052 | 3474 | TTCTCTTGTGAGGTGTGTGGGGCAAGGTTTactcagaaaggtaacctcctccgccacattaaaCTG | 46F_47S_49E_50V_52G_53A_54R_55F_67L_ | 2.600867303 |
| 4053 | 61 | TTCGCGTGTGAAATTTGTGGGGCATGTTTTactcagaaaggtaacctcctccgccacattaaaCTG | 46F_47A_49E_50I_52G_53A_54C_55F_67L_ | 2.599768003 |
| 4054 | 1301 | TTCGGGTGTGAAGTTTGTGGGGCATGTTTTactcagaaaggtaacctcctccgccacattaaaCTG | 46F_47G_49E_50V_52G_53A_54C_55F_67L_ | 2.598857699 |
| 4055 | 1277 | TTCGGATGTGAAATTTGTGGGGCAGTGTTTactcagaaaggtaacctcctccgccacattaaaCTG | 46F_47G_49E_50I_52G_53A_54V_55F_67L_ | 2.597851651 |
| 4056 | 472 | TTCTGCTGTCCTGTTTGTGGGGCACCGTTTactcagaaaggtaacctcctccgccacattaaaCTG | 46F_47C_49P_50V_52G_53A_54P_55F_67L_ | 2.597266706 |
| 4057 | 6716 | CTGCAATGCCCCATCTGCGGGTTTACATGTactcagaaaggtaacctcctccgccacattaaaCTG | 46L_47Q_49P_50I_52G_53F_54T_55C_67L_ | 2.593622488 |
| 4058 | 4457 | TTCTATTGTAACATCTGTGGGGCAGTCTTTactcagaaaggtaacctcctccgccacattaaaCTG | 46F_47Y_49N_50I_52G_53A_54V_55F_67L_ | 2.592821958 |
| 4059 | 966 | TTCTTTTGTGACATTTGTGGGGCACATTTTactcagaaaggtaacctcctccgccacattaaaCTG | 46F_47F_49D_50I_52G_53A_54H_55F_67L_ | 2.592451511 |
| 4060 | 4114 | TTCTGGTGTGATCAATGTGGGGCACGCTTTactcagaaaggtaacctcctccgccacattaaaCTG | 46F_47W_49D_50Q_52G_53A_54R_55F_67L_ | 2.592019036 |
| 4061 | 3834 | TTCACTTGTCCTGTTTGTGGGGCACGCTTTactcagaaaggtaacctcctccgccacattaaaCTG | 46F_47T_49P_50V_52G_53A_54R_55F_67L_ | 2.589635472 |
| 4062 | 6473 | CTTGCCTGCCCAATTTGTGGGTTTCAGTGCactcagaaaggtaacctcctccgccacattaaaCTG | 46L_47A_49P_50I_52G_53F_54Q_55C_67L_ | 2.586264615 |
| 4063 | 1276 | TTCGGATGTGAGATCTGTGGGGCAACGTTTactcagaaaggtaacctoctccgccacattaaaCTG | 46F_47G_49E_50I_52G_53A_54T_55F_67L_ | 2.586106203 |
| 4064 | 1260 | TTCGGATGTGAGATCTGTGGGGCAGCGTTTactcagaaaggtaacctcctccgccacattaaaCTG | 46F_47G_49E_50I_52G_53A_54A_55F_67L_ | 2.58586462 |
| 4065 | 4137 | TTCTGGTGTGACGTGTGTGGGGCAGTCTTTactcagaaaggtaacctcctccgccacattaaaCTG | 46F_47W_49D_50V_52G_53A_54V_55F_67L_ | 2.584868726 |
| 4066 | 313 | TTCTGTTGTGAGATATGTGGGGCACAATTTactcagaaaggtaacctcctccgccacattaaaCTG | 46F_47C_49E_50I_52G_53A_54Q_55F_67L_ | 2.583272023 |
| 4067 | 7933 | CTGGTGTGTGAAATATGCGGCTTCCAATGTactcagaaaggtaacctcctcogccacattaaaCTG | 46L_47V_49E_50I_52G_53F_54Q_55C_67L_ | 2.583151418 |
| 4068 | 2956 | TTCCAATGTGAGATTTGTGGGGCAACATTTactcagaaaggtaacctcctccgccacattaaaCTG | 46F_47Q_49E_50I_52G_53A_54T_55F_67L_ | 2.58308496 |
| 4069 | 1000 | TTCTTTTGTGACGTGTGTGGGGCAGCGTTTactcagaaaggtaacctcctccgccacattaaaCTG | 46F_47F_49D_50V_52G_53A_54A_55F_67L_ | 2.582329783 |
| 4070 | 232 | TTCGCTTGTCCTGTGTGTGGGGCACCCTTTactcagaaaggtaacctcctccgccacattaaaCTG | 46F_47A_49P_50V_52G_53A_54P_55F_67L_ | 2.581740569 |
| 4071 | 6779 | CTAACCTGTCCCATATGTGGTTTTTATTGCactcagaaaggtaacctcctccgccacattaaaCTG | 46L_47T_49P_50I_52G_53F_54Y_55C_67L_ | 2.581123157 |
| 4072 | 7573 | TTCTGGTGTGAAATTTGTGGCCGCCAGTTTactcagaaaggtaacctcctccgccacattaaaCTG | 46F_47W_49E_50I_52G_53R_54Q_55F_67L_ | 2.57878546 |
| 4073 | 460 | TTCTGTTGTCCTGTGTGTGGGGCAGCCTTTactcagaaaggtaacctcctccgccacattaaaCTG | 46F_47C_49P_50V_52G_53A_54A_55F_67L_ | 2.577887995 |
| 4074 | 6613 | CTGATTTGCCCAATCTGTGGCTTTCAGTGTactcagaaaggtaacctcctccgccacattaaaCTG | 46L_47I_49P_50I_52G_53F_54Q_55C_67L_ | 2.573696118 |
| 4075 | 1905 | TTCATTTGTCCGGTCTGTGGGGCAGGCTTTactcagaaaggtaacctcctccgccacattaaaCTG | 46F_47I_49P_50V_52G_53A_54G_55F_67L_ | 2.572366402 |
| 4076 | 6766 | CTCACATGCCCAATCTGTGGGTTCCACTGTactcagaaaggtaacctcctccgccacattaaaCTG | 46L_47T_49P_50I_52G_53F_54H_55C_67L_ | 2.572187751 |
| 4077 | 6754 | CTAAGCTGCCCAATATGCGGGTTCCGGTGTactcagaaaggtaacctcctccgccacattaaaCTG | 46L_47S_49P_50I_52G_53F_54R_55C_67L_ | 2.5712282 |
| 4078 | 3793 | TTCACCTGTCCAATCTGTGGGGCACAGTTTactcagaaaggtaacctcctccgccacattaaaCTG | 46F_47T_49P_50I_52G_53A_54Q_55F_67L_ | 2.568402784 |
| 4079 | 4550 | TTCTATTGTCCAGTGTGTGGGGCAATGTTTactcagaaaggtaacctcctccgccacattaaaCTG | 46F_47Y_49P_50V_52G_53A_54M_55F_67L_ | 2.568000359 |
| 4080 | 3436 | TTCTCCTGTGAAATTTGTGGGGCAACTTTTactcagaaaggtaacctcctccgccacattaaaCTG | 46F_47S_49E_50I_52G_53A_54T_55F_67L_ | 2.565143701 |
| 4081 | 3913 | TTCGTGTGTGAGATCTGTGGGGCACAGTTTactcagaaaggtaacctcctccgccacattaaaCTG | 46F_47V_49E_50I_52G_53A_54Q_55F_67L_ | 2.563798175 |
| 4082 | 3070 | TTCCAATGTCCAATCTGTGGGGCAATGTTTactcagaaaggtaacctcctccgccacattaaaCTG | 46F_47Q_49P_50I_52G_53A_54M_55F_67L_ | 2.563439713 |
| 4083 | 4304 | TTCTGGTGTCCTGTTTGTGGGGCATTTTTTactcagaaaggtaacctcctccgccacattaaaCTG | 46F_47W_49P_50V_52G_53A_54F_55F_67L_ | 2.562234573 |
| 4084 | 6823 | TTGTATTGTCCGATCTGCGGGTTCGAGTGCactcagaaaggtaacctcctccgccacattaaaCTG | 46L_47Y_49P_50I_52G_53F_54E_55C_67L_ | 2.560137574 |
| 4085 | 6805 | CTGTGGTGTCCAATCTGCGGTTTTGGCTGCactcagaaaggtaacctcctccgccacattaaaCTG | 46L_47W_49P_50I_52G_53F_54G_55C_67L_ | 2.558900161 |
| 4086 | 2993 | TTCCAGTGTGAGGTTTGTGGGGCACAGTTTactcagaaaggtaacctcctccgccacattaaaCTG | 46F_47Q_49E_50V_52G_53A_54Q_55F_67L_ | 2.558639592 |
| 4087 | 6665 | CTTATGTGTCCAATTTGTGGTTTCGGATGCactcagaaaggtaacctcctccgccacattaaaCTG | 46L_47M_49P_50I_52G_53F_54G_55C_67L_ | 2.557826066 |
| 4088 | 3945 | TTCGTGTGTGAAGTCTGTGGGGCAGGCTTTactcagaaaggtaacctcctccgccacattaaaCTG | 46F_47V_49E_50V_52G_53A_54G_55F_67L_ | 2.557323233 |
| 4089 | 1094 | TTCTTTTGTAATATATGTGGGGCAAGGTTTactcagaaaggtaacctcctcogccacattaaaCTG | 46F_47F_49N_50I_52G_53A_54R_55F_67L_ | 2.556090052 |
| 4090 | 1147 | TTCTTTTGTCCCATCTGTGGGGCAATCTTTactcagaaaggtaacctcctccgccacattaaaCTG | 46F_47F_49P_50I_52G_53A_54I_55F_67L_ | 2.554582053 |
| 4091 | 1673 | TTCCATTGTCCAGTGTGTGGGGCACAATTTactcagaaaggtaacctcctccgccacattaaaCTG | 46F_47H_49P_50V_52G_53A_54Q_55F_67L_ | 2.55352032 |
| 4092 | 3580 | TTCAGTTGTCCCGTCTGTGGGGCAGCCTTTactcagaaaggtaacctcctccgccacattaaaCTG | 46F_47S_49P_50V_52G_53A_54A_55F_67L_ | 2.552360733 |
| 4093 | 3614 | TTCACCTGTGATATTTGTGGGGCACGATTTactcagaaaggtaacctcctccgccacattaaaCTG | 46F_47T_49D_50I_52G_53A_54R_55F_67L_ | 2.552257393 |
| 4094 | 1095 | TTCTTTTGTAATATTTGTGGGGCATCCTTTactcagaaaggtaacctcctccgccacattaaaCTG | 46F_47F_49N_50I_52G_53A_54S_55F_67L_ | 2.550413644 |
| 4095 | 7934 | CTCGTCTGCGAGATTTGTGGCTTCAGATGCactcagaaaggtaacctcctccgccacattaaaCTG | 46L_47V_49E_50I_52G_53F_54R_55C_67L_ | 2.549408757 |
| 4096 | 960 | TTCTTCTGTGACATCTGTGGGGCAGCCTTTactcagaaaggtaacctcctccgccacattaaaCTG | 46F_47F_49D_50I_52G_53A_54A_55F_67L_ | 2.547843358 |
| 4097 | 1635 | TTCCACTGTCCTATCTGTGGGGCATCCTTTactcagaaaggtaacctcctccgccacattaaaCTG | 46F_47H_49P_50I_52G_53A_54S_55F_67L_ | 2.54416442 |
| 4098 | 1001 | TTCTTTTGTGACGTATGTGGGGCATGCTTTactcagaaaggtaacctcctccgccacattaaaCTG | 46F_47F_49D_50V_52G_53A_54C_55F_67L_ | 2.542056092 |
| 4099 | 4160 | TTCTGGTGTGAGCAGTGTGGGGCAGCTTTTactcagaaaggtaacctcctccgccacattaaaCTG | 46F_47W_49E_50Q_52G_53A_54A_55F_67L_ | 2.541368327 |
| 4100 | 7856 | CTGCAGTGCGAGATTTGTGGCTTCACATGCactcagaaaggtaacctcctccgccacattaaaCTG | 46L_47Q_49E_50I_52G_53F_54T_55C_67L_ | 2.54122345 |
| 4101 | 1316 | TTCGGTTGTGAAGTCTGTGGGGCAACCTTTactcagaaaggtaacctcctccgccacattaaaCTG | 46F_47G_49E_50V_52G_53A_54T_55F_67L_ | 2.539889591 |
| 4102 | 1791 | TTCATTTGTGAGGTGTGTGGGGCAAACTTTactcagaaaggtaacctcctccgccacattaaaCTG | 46F_47I_49E_50V_52G_53A_54N_55F_67L_ | 2.538560365 |
| 4103 | 1745 | TTCATTTGTGAAATCTGTGGGGCAGGGTTTactcagaaaggtaacctcctccgccacattaaaCTG | 46F_47I_49E_50I_52G_53A_54G_55F_67L_ | 2.53842332 |
| 4104 | 2712 | TTCAATTGTGAAATATGTGGGGCACCTTTTactcagaaaggtaacctcctccgccacattaaaCTG | 46F_47N_49E_50I_52G_53A_54P_55F_67L_ | 2.538254294 |
| 4105 | 1397 | TTCGGGTGTCCTATCTGTGGGGCAGTGTTTactcagaaaggtaacctcctccgccacattaaaCTG | 46F_47G_49P_50I_52G_53A_54V_55F_67L_ | 2.536031341 |
| 4106 | 1149 | TTCTTTTGTCCTATCTGTGGGGCACTATTTactcagaaaggtaacctcctccgccacattaaaCTG | 46F_47F_49P_50I_52G_53A_54L_55F_67L_ | 2.535435373 |
| 4107 | 1501 | TTCCATTGTGAGATTTGTGGGGCATGCTTTactcagaaaggtaacctcctccgccacattaaaCTG | 46F_47H_49E_50I_52G_53A_54C_55F_67L_ | 2.535020208 |
| 4108 | 1675 | TTCCATTGTCCAGTTTGTGGGGCATCATTTactcagaaaggtaacctcctccgccacattaaaCTG | 46F_47H_49P_50V_52G_53A_54S_55F_67L_ | 2.53222177 |
| 4109 | 7895 | CTGAGTTGCGAAATCTGCGGCTTCAGCTGTactcagaaaggtaacctcctccgccacattaaaCTG | 46L_47S_49E_50I_52G_53F_54S_55C_67L_ | 2.532140777 |
| 4110 | 4533 | TTCTACTGTCCCCAGTGTGGGGCACAGTTTactcagaaaggtaacctcctccgccacattaaaCTG | 46F_47Y_49P_50Q_52G_53A_54Q_55F_67L_ | 2.530727308 |
| 4111 | 7964 | CTGTACTGCGAAATCTGCGGGTTTTTTTGCactcagaaaggtaacctcctccgccacattaaaCTG | 46L_47Y_49E_50I_52G_53F_54F_55C_67L_ | 2.530551511 |
| 4112 | 194 | TTCGCATGTCCTATTTGTGGGGCACGATTTactcagaaaggtaacctcctccgccacattaaaCTG | 46F_47A_49P_50I_52G_53A_54R_55F_67L_ | 2.526509117 |
| 4113 | 3433 | TTCTCGTGTGAAATCTGTGGGGCACAGTTTactcagaaaggtaacctcctccgccacattaaaCTG | 46F_47S_49E_50I_52G_53A_54Q_55F_67L_ | 2.524582209 |
| 4114 | 3701 | TTCACTTGTGAGGTCTGTGGGGCATGTTTTactcagaaaggtaacctcctccgccacattaaaCTG | 46F_47T_49E_50V_52G_53A_54C_55F_67L_ | 2.522575794 |
| 4115 | 1144 | TTCTTCTGTCCTATTTGTGGGGCATTTTTTactcagaaaggtaacctcctccgccacattaaaCTG | 46F_47F_49P_50I_52G_53A_54F_55F_67L_ | 2.521394769 |
| 4116 | 6781 | CTCGTCTGCCCTATATGCGGCTTCTGCTGCactcagaaaggtaacctcctccgccacattaaaCTG | 46L_47V_49P_50I_52G_53F_54C_55C_67L_ | 2.517927361 |
| 4117 | 308 | TTCTGCTGTGAGATCTGTGGGGCAAAGTTTactcagaaaggtaacctcctccgccacattaaaCTG | 46F_47C_49E_50I_52G_53A_54K_55F_67L_ | 2.516260215 |
| 4118 | 72 | TTCGCATGTGAAATATGTGGGGCACCATTTactcagaaaggtaacctcctccgccacattaaaCTG | 46F_47A_49E_50I_52G_53A_54P_55F_67L_ | 2.514061094 |
| 4119 | 2880 | TTCCAATGTGATATTTGTGGGGCAGCCTTTactcagaaaggtaacctcctccgccacattaaaCTG | 46F_47Q_49D_50I_52G_53A_54A_55F_67L_ | 2.513763915 |
| 4120 | 6584 | CTGCACTGCCCAATATGTGGCTTCTTTTGTactcagaaaggtaacctcctccgccacattaaaCTG | 46L_47H_49P_50I_52G_53F_54F_55C_67L_ | 2.512272451 |
| 4121 | 4183 | TTCTGGTGTGAAGTGTGTGGGGCAGAGTTTactcagaaaggtaacctcctccgccacattaaaCTG | 46F_47W_49E_50V_52G_53A_54E_55F_67L_ | 2.509971452 |
| 4122 | 3821 | TTCACTTGTCCAGTGTGTGGGGCATGTTTTactcagaaaggtaacctcctccgccacattaaaCTG | 46F_47T_49P_50V_52G_53A_54C_55F_67L_ | 2.507787824 |
| 4123 | 1387 | TTCGGTTGTCCCATCTGTGGGGCAATTTTTactcagaaaggtaacctcctccgccacattaaaCTG | 46F_47G_49P_50I_52G_53A_54I_55F_67L_ | 2.505652264 |
| 4124 | 4413 | TTCTACTGTGAACAGTGTGGGGCACAATTTactcagaaaggtaacctcctccgccacattaaaCTG | 46F_47Y_49E_50Q_52G_53A_540_55F_67L_ | 2.503028087 |
| 4125 | 4263 | TTCTGGTGTCCTATTTGTGGGGCAGAGTTTactcagaaaggtaacctcctccgccacattaaaCTG | 46F_47W_49P_50I_52G_53A_54E_55F_67L_ | 2.50246943 |
| 4126 | 3796 | TTCACATGTCCAATCTGTGGGGCAACCTTTactcagaaaggtaacctcctccgccacattaaaCTG | 46F_47T_49P_50I_52G_53A_54T_55F_67L_ | 2.501886854 |
| 4127 | 4286 | TTCTGGTGTCCACAATGTGGGGCACATTTTactcagaaaggtaacctcctccgccacattaaaCTG | 46F_47W_49P_50Q_52G_53A_54H_55F_67L_ | 2.499800941 |
| 4128 | 1634 | TTCCACTGTCCTATTTGTGGGGCACGTTTTactcagaaaggtaacctcctccgccacattaaaCTG | 46F_47H_49P_50I_52G_53A_54R_55F_67L_ | 2.497821227 |
| 4129 | 4514 | TTCTATTGTCCGATCTGTGGGGCAAGATTTactcagaaaggtaacctcctccgccacattaaaCTG | 46F_47Y_49P_50I_52G_53A_54R_55F_67L_ | 2.496107627 |
| 4130 | 6773 | CTGACGTGCCCTATTTGTGGCTTCCAGTGTactcagaaaggtaacctcctccgccacattaaaCTG | 46L_47T_49P_50I_52G_53F_54Q_55C_67L_ | 2.495188325 |
| 4131 | 1852 | TTCATCTGTAACGTCTGTGGGGCACCTTTTactcagaaaggtaacctcctccgccacattaaaCTG | 46F_47I_49N_50V_52G_53A_54P_55F_67L_ | 2.487053876 |
| 4132 | 6464 | TTGGCGTGTCCCATCTGTGGCTTTTTTTGCactcagaaaggtaacctcctccgccacattaaaCTG | 46L_47A_49P_50I_52G_53F_54F_55C_67L_ | 2.486511477 |
| 4133 | 4210 | TTCTGGTGTAATATTTGTGGGGCAATGTTTactcagaaaggtaacctcctccgccacattaaaCTG | 46F_47W_49N_50I_52G_53A_54M_55F_67L_ | 2.486324805 |
| 4134 | 6660 | CTTATGTGTCCAATCTGCGGTTTTGCATGTactcagaaaggtaacctcctccgccacattaaaCTG | 46L_47M_49P_50I_52G_53F_54A_55C_67L_ | 2.482765471 |
| 4135 | 3840 | TTCGTGTGTGACATCTGTGGGGCAGCCTTTactcagaaaggtaacctcctccgccacattaaaCTG | 46F_47V_49D_50I_52G_53A_54A_55F_67L_ | 2.482710316 |
| 4136 | 2500 | TTCATGTGTGAGGTATGTGGGGCAGCTTTTactcagaaaggtaacctcctccgccacattaaaCTG | 46F_47M_49E_50V_52G_53A_54A_55F_67L_ | 2.482067949 |
| 4137 | 4219 | TTCTGGTGTAATATCTGTGGGGCATATTTTactcagaaaggtaacctcctccgccacattaaaCTG | 46F_47W_49N_50I_52G_53A_54Y_55F_67L_ | 2.480728028 |
| 4138 | 3113 | TTCCAGTGTCCCGTGTGTGGGGCACAGTTTactcagaaaggtaacctcctccgccacattaaaCTG | 46F_47Q_49P_50V_52G_53A_54Q_55F_67L_ | 2.478910447 |
| 4139 | 1631 | TTCCATTGTCCAATTTGTGGGGCAAACTTTactcagaaaggtaacctcctccgccacattaaaCTG | 46F_47H_49P_50I_52G_53A_54N_55F_67L_ | 2.476998743 |
| 4140 | 3421 | TTCAGCTGTGAAATCTGTGGGGCATGCTTTactcagaaaggtaacctcctccgccacattaaaCTG | 46F_47S_49E_50I_52G_53A_54C_55F_67L_ | 2.474777722 |
| 4141 | 7936 | CTGGTGTGTGAAATCTGCGGGTTCACATGTactcagaaaggtaacctcctccgccacattaaaCTG | 46L_47V_49E_50I_52G_53F_54T_55C_67L_ | 2.473154531 |
| 4142 | 4090 | TTCTGGTGTGACATTTGTGGGGCAATGTTTactcagaaaggtaacctcctccgccacattaaaCTG | 46F_47W_49D_50I_52G_53A_54M_55F_67L_ | 2.471639883 |
| 4143 | 4034 | TTCGTCTGTCCAATATGTGGGGCACGGTTTactcagaaaggtaacctcctccgccacattaaaCTG | 46F_47V_49P_50I_52G_53A_54R_55F_67L_ | 2.471391343 |
| 4144 | 7900 | CTAACCTGCGAGATATGTGGGTTTGCATGCactcagaaaggtaacctcctccgccacattaaaCTG | 46L_47T_49E_50I_52G_53F_54A_55C_67L_ | 2.47054839 |
| 4145 | 4454 | TTCTATTGTAACATCTGTGGGGCAAGATTTactcagaaaggtaacctcctccgccacattaaaCTG | 46F_47Y_49N_50I_52G_53A_54R_55F_67L_ | 2.467319389 |
| 4146 | 2580 | TTCATGTGTCCAATTTGTGGGGCAGCCTTTactcagaaaggtaacctcctccgccacattaaaCTG | 46F_47M_49P_50I_52G_53A_54A_55F_67L_ | 2.466050147 |
| 4147 | 7959 | TTGTGGTGCGAGATCTGCGGCTTCTATTGCactcagaaaggtaacctcctccgccacattaaaCTG | 46L_47W_49E_50I_52G_53F_54Y_55C_67L_ | 2.465969188 |
| 4148 | 1054 | TTCTTTTGTGAGCAGTGTGGGGCAAGGTTTactcagaaaggtaacctcctccgccacattaaaCTG | 46F_47F_49E_50Q_52G_53A_54R_55F_67L_ | 2.46533421 |
| 4149 | 7951 | CTGTGGTGTGAGATTTGTGGATTCAATTGCactcagaaaggtaacctcctccgccacattaaaCTG | 46L_47W_49E_50I_52G_53F_54N_55C_67L_ | 2.457825935 |
| 4150 | 3831 | TTCACATGTCCCGTGTGTGGGGCAAACTTTactcagaaaggtaacctcctccgccacattaaaCTG | 46F_47T_49P_50V_52G_53A_54N_55F_67L_ | 2.457761253 |
| 4151 | 6586 | CTTCATTGCCCAATTTGCGGATTCCACTGTactcagaaaggtaacctcctccgccacattaaaCTG | 46L_47H_49P_50I_52G_53F_54H_55C_67L_ | 2.454677625 |
| 4152 | 2272 | TTCCTCTGTGAAGTCTGTGGGGCACCCTTTactcagaaaggtaacctcctccgccacattaaaCTG | 46F_47L_49E_50V_52G_53A_54P_55F_67L_ | 2.44992775 |
| 4153 | 4337 | TTCTACTGTGATATATGTGGGGCAGTTTTTactcagaaaggtaacctcctccgccacattaaaCTG | 46F_47Y_49D_50I_52G_53A_54V_55F_67L_ | 2.44781575 |
| 4154 | 2595 | TTCATGTGTCCTATCTGTGGGGCATCTTTTactcagaaaggtaacctcctccgccacattaaaCTG | 46F_47M_49P_50I_52G_53A_54S_55F_67L_ | 2.447796523 |
| 4155 | 2460 | TTCATGTGTGAGATCTGTGGGGCAGCTTTTactcagaaaggtaacctcctccgccacattaaaCTG | 46F_47M_49E_50I_52G_53A_54A_55F_67L_ | 2.445307672 |
| 4156 | 1661 | TTCCACTGTCCTGTATGTGGGGCATGTTTTactcagaaaggtaacctcctccgccacattaaaCTG | 46F_47H_49P_50V_52G_53A_54C_55F_67L_ | 2.444462227 |
| 4157 | 195 | TTCGCCTGTCCTATCTGTGGGGCATCATTTactcagaaaggtaacctcctccgccacattaaaCTG | 46F_47A_49P_50I_52G_53A_54S_55F_67L_ | 2.443254877 |
| 4158 | 4481 | TTCTATTGTAACGTGTGTGGGGCATGCTTTactcagaaaggtaacctcctccgccacattaaaCTG | 46F_47Y_49N_50V_52G_53A_54C_55F_67L_ | 2.442896065 |
| 4159 | 1008 | TTCTTCTGTGATGTGTGTGGGGCAAAATTTactcagaaaggtaacctcctccgccacattaaaCTG | 46F_47F_49D_50V_52G_53A_54K_55F_67L_ | 2.44154888 |
| 4160 | 1317 | TTCGGATGTGAAGTCTGTGGGGCAGTGTTTactcagaaaggtaacctcctccgccacattaaaCTG | 46F_47G_49E_50V_52G_53A_54V_55F_67L_ | 2.44016267 |
| 4161 | 475 | TTCTGTTGTCCAGTCTGTGGGGCATCCTTTactcagaaaggtaacctcctccgccacattaaaCTG | 46F_47C_49P_50V_52G_53A_54S_55F_67L_ | 2.435791439 |
| 4162 | 4071 | TTCGTGTGTCCTGTGTGTGGGGCAAATTTTactcagaaaggtaacctcctccgccacattaaaCTG | 46F_47V_49P_50V_52G_53A_54N_55F_67L_ | 2.433596884 |
| 4163 | 4280 | TTCTGGTGTCCCCAGTGTGGGGCAGCTTTTactcagaaaggtaacctcctccgccacattaaaCTG | 46F_47W_49P_50Q_52G_53A_54A_55F_67L_ | 2.430973525 |
| 4164 | 4161 | TTCTGGTGTGAGCAGTGTGGGGCATGCTTTactcagaaaggtaacctcctccgccacattaaaCTG | 46F_47W_49E_50Q_52G_53A_54C_55F_67L_ | 2.429847438 |
| 4165 | 348 | TTCTGCTGTGAGGTCTGTGGGGCAAAATTTactcagaaaggtaacctcctccgccacattaaaCTG | 46F_47C_49E_50V_52G_53A_54K_55F_67L_ | 2.429428638 |
| 4166 | 3914 | TTCGTGTGTGAGATATGTGGGGCACGCTTTactcagaaaggtaacctcctccgccacattaaaCTG | 46F_47V_49E_50I_52G_53A_54R_55F_67L_ | 2.428257777 |
| 4167 | 6708 | CTGCAGTGTCCGATCTGTGGTTTCAAGTGTactcagaaaggtaacctcctccgccacattaaaCTG | 46L_47Q_49P_50I_52G_53F_54K_55C_67L_ | 2.426283124 |
| 4168 | 6765 | CTCACTTGTCCGATTTGCGGTTTCGGATGTactcagaaaggtaacctcctccgccacattaaaCTG | 46L_47T_49P_50I_52G_53F_54G_55C_67L_ | 2.423944953 |
| 4169 | 1514 | TTCCATTGTGAAATATGTGGGGCACGATTTactcagaaaggtaacctcctccgccacattaaaCTG | 46F_47H_49E_50I_52G_53A_54R_55F_67L_ | 2.421350355 |
| 4170 | 6493 | CTGTGTTGTCCGATCTGCGGCTTTCAGTGCactcagaaaggtaacctcctccgccacattaaaCTG | 46L_47C_49P_50I_52G_53F_54Q_55C_67L_ | 2.418389583 |
| 4171 | 134 | TTCGCCTGTAACATTTGTGGGGCAAGGTTTactcagaaaggtaacctcctccgccacattaaaCTG | 46F_47A_49N_50I_52G_53A_54R_55F_67L_ | 2.417131325 |
| 4172 | 4416 | TTCTACTGTGAACAGTGTGGGGCAACCTTTactcagaaaggtaacctcctccgccacattaaaCTG | 46F_47Y_49E_50Q_52G_53A_54T_55F_67L_ | 2.414263277 |
| 4173 | 4130 | TTCTGGTGTGATGTGTGTGGGGCAATGTTTactcagaaaggtaacctcctccgccacattaaaCTG | 46F_47W_49D_50V_52G_53A_54M_55F_67L_ | 2.412577973 |
| 4174 | 3340 | TTCAGGTGTCCAGTATGTGGGGCAGCTTTTactcagaaaggtaacctcctccgccacattaaaCTG | 46F_47R_49P_50V_52G_53A_54A_55F_67L_ | 2.409148131 |
| 4175 | 474 | TTCTGCTGTCCCGTTTGTGGGGCAAGATTTactcagaaaggtaacctcctccgccacattaaaCTG | 46F_47C_49P_50V_52G_53A_54R_55F_67L_ | 2.409051985 |
| 4176 | 1079 | TTCTTCTGTGAGGTGTGTGGGGCATACTTTactcagaaaggtaacctcctccgccacattaaaCTG | 46F_47F_49E_50V_52G_53A_54Y_55F_67L_ | 2.407613446 |
| 4177 | 1436 | TTCGGCTGTCCGGTGTGTGGGGCAACGTTTactcagaaaggtaacctcctccgccacattaaaCTG | 46F_47G_49P_50V_52G_53A_54T_55F_67L_ | 2.405017678 |
| 4178 | 7734 | CTCCACTGTGAGATTTGTGGGTTCCGATGCactcagaaaggtaacctcctccgccacattaaaCTG | 46L_47H_49E_50I_52G_53F_54R_55C_67L_ | 2.403676937 |
| 4179 | 1437 | TTCGGCTGTCCGGTGTGTGGGGCAGTGTTTactcagaaaggtaacctcctccgccacattaaaCTG | 46F_47G_49P_50V_52G_53A_54V_55F_67L_ | 2.402072901 |
| 4180 | 1660 | TTCCACTGTCCTGTTTGTGGGGCAGCATTTactcagaaaggtaacctcctccgccacattaaaCTG | 46F_47H_49P_50V_52G_53A_54A_55F_67L_ | 2.400353829 |
| 4181 | 3554 | TTCAGCTGTCCCATTTGTGGGGCAAGATTTactcagaaaggtaacctcctccgccacattaaaCTG | 46F_47S_49P_50I_52G_53A_54R_55F_67L_ | 2.397837981 |
| 4182 | 6594 | TTGCATTGTCCCATCTGTGGTTTCCGTTGTactcagaaaggtaacctcctccgccacattaaaCTG | 46L_47H_49P_50I_52G_53F_54R_55C_67L_ | 2.397070558 |
| 4183 | 1396 | TTCGGATGTCCCATCTGTGGGGCAACATTTactcagaaaggtaacctcctccgccacattaaaCTG | 46F_47G_49P_50I_52G_53A_54T_55F_67L_ | 2.396268432 |
| 4184 | 3476 | TTCAGCTGTGAGGTTTGTGGGGCAACTTTTactcagaaaggtaacctcctccgccacattaaaCTG | 46F_47S_49E_50V_52G_53A_54T_55F_67L_ | 2.396182655 |
| 4185 | 7872 | CTCCGGTGCGAGATTTGTGGATTTCCCTGCactcagaaaggtaacctcctccgccacattaaaCTG | 46L_47R_49E_50I_52G_53F_54P_55C_67L_ | 2.395682256 |
| 4186 | 4372 | TTCTACTGTGATGTCTGTGGGGCACCGTTTactcagaaaggtaacctcctccgccacattaaaCTG | 46F_47Y_49D_50V_52G_53A_54P_55F_67L_ | 2.393517003 |
| 4187 | 6799 | CTGGTGTGTCCTATTTGTGGCTTTTACTGCactcagaaaggtaacctcctccgccacattaaaCTG | 46L_47V_49P_50I_52G_53F_54Y_55C_67L_ | 2.392934724 |
| 4188 | 77 | TTCGCGTGTGAGATCTGTGGGGCAGTGTTTactcagaaaggtaacctcctccgccacattaaaCTG | 46F_47A_49E_50I_52G_53A_54V_55F_67L_ | 2.392349906 |
| 4189 | 4495 | TTCTACTGTAACGTTTGTGGGGCAAGCTTTactcagaaaggtaacctcctccgccacattaaaCTG | 46F_47Y_49N_50V_52G_53A_54S_55F_67L_ | 2.391573948 |
| 4190 | 4547 | TTCTACTGTCCAGTGTGTGGGGCAATCTTTactcagaaaggtaacctcctccgccacattaaaCTG | 46F_47Y_49P_50V_52G_53A_54I_55F_67L_ | 2.391485921 |
| 4191 | 4305 | TTCTGGTGTCCCGTGTGTGGGGCAGGTTTTactcagaaaggtaacctcctccgccacattaaaCTG | 46F_47W_49P_50V_52G_53A_54G_55F_67L_ | 2.388040416 |
| 4192 | 4493 | TTCTACTGTAATGTATGTGGGGCACAGTTTactcagaaaggtaacctcctccgccacattaaaCTG | 46F_47Y_49N_50V_52G_53A_54Q_55F_67L_ | 2.387741721 |
| 4193 | 3594 | TTCTCTTGTCCAGTATGTGGGGCACGATTTactcagaaaggtaacctcctccgccacattaaaCTG | 46F_47S_49P_50V_52G_53A_54R_55F_67L_ | 2.381505421 |
| 4194 | 1392 | TTCGGCTGTCCCATCTGTGGGGCACCTTTTactcagaaaggtaacctcctccgccacattaaaCTG | 46F_47G_49P_50I_52G_53A_54P_55F_67L_ | 2.379288479 |
| 4195 | 1554 | TTCCACTGTGAGGTTTGTGGGGCACGCTTTactcagaaaggtaacctcctccgccacattaaaCTG | 46F_47H_49E_50V_52G_53A_54R_55F_67L_ | 2.378575082 |
| 4196 | 73 | TTCGCCTGTGAGATTTGTGGGGCACAATTTactcagaaaggtaacctcctccgccacattaaaCTG | 46F_47A_49E_50I_52G_53A_54Q_55F_67L_ | 2.37515419 |
| 4197 | 3592 | TTCAGCTGTCCTGTATGTGGGGCACCGTTTactcagaaaggtaacctcctccgccacattaaaCTG | 46F_47S_49P_50V_52G_53A_54P_55F_67L_ | 2.372058671 |
| 4198 | 3180 | TTCCGATGTGAGATATGTGGGGCAGCCTTTactcagaaaggtaacctcctccgccacattaaaCTG | 46F_47R_49E_50I_52G_53A_54A_55F_67L_ | 2.371717412 |
| 4199 | 4531 | TTCTATTGTCCACAATGTGGGGCAAATTTTactcagaaaggtaacctcctccgccacattaaaCTG | 46F_47Y_49P_50Q_52G_53A_54N_55F_67L_ | 2.370383074 |
| 4200 | 3783 | TTCACCTGTCCCATCTGTGGGGCAGAGTTTactcagaaaggtaacctcctccgccacattaaaCTG | 46F_47T_49P_50I_52G_53A_54E_55F_67L_ | 2.370350512 |
| 4201 | 7716 | CTGGGGTGTGAGATCTGCGGGTTCACTTGCactcagaaaggtaacctcctccgccacattaaaCTG | 46L_47G_49E_50I_52G_53F_54T_55C_67L_ | 2.370022376 |
| 4202 | 7593 | TTTTATTGTGAGATCTGTGGCAGGCAATTTactcagaaaggtaacctcctccgccacattaaaCTG | 46F_47Y_49E_50I_52G_53R_54Q_55F_67L_ | 2.366827617 |
| 4203 | 7635 | CTGTGTTGCGAGATTTGCGGCTTCAGTTGTactcagaaaggtaacctcctccgccacattaaaCTG | 46L_47C_49E_50I_52G_53F_54S_55C_67L_ | 2.365674813 |
| 4204 | 1435 | TTCGGCTGTCCGGTGTGTGGGGCATCATTTactcagaaaggtaacctcctccgccacattaaaCTG | 46F_47G_49P_50V_52G_53A_54S_55F_67L_ | 2.363487639 |
| 4205 | 6741 | CTGAGCTGTCCCATCTGTGGCTTCTGCTGCactcagaaaggtaacctcctccgccacattaaaCTG | 46L_47S_49P_50I_52G_53F_54C_55C_67L_ | 2.360304 |
| 4206 | 360 | TTCTGTTGTAATATCTGTGGGGCAGCCTTTactcagaaaggtaacctcctccgccacattaaaCTG | 46F_47C_49N_50I_52G_53A_54A_55F_67L_ | 2.360066553 |
| 4207 | 1313 | TTCGGTTGTGAGGTTTGTGGGGCACAGTTTactcagaaaggtaacctcctccgccacattaaaCTG | 46F_47G_49E_50V_52G_53A_54Q_55F_67L_ | 2.360044085 |
| 4208 | 3661 | TTCACATGTGAAATTTGTGGGGCATGCTTTactcagaaaggtaacctcctccgccacattaaaCTG | 46F_47T_49E_50I_52G_53A_54C_55F_67L_ | 2.358505124 |
| 4209 | 1892 | TTCATCTGTCCACAATGTGGGGCACCATTTactcagaaaggtaacctcctccgccacattaaaCTG | 46F_47I_49P_50Q_52G_53A_54P_55F_67L_ | 2.357241941 |
| 4210 | 1150 | TTCTTCTGTCCCATTTGTGGGGCAATGTTTactcagaaaggtaacctcctccgccacattaaaCTG | 46F_47F_49P_50I_52G_53A_54M_55F_67L_ | 2.357040916 |
| 4211 | 6616 | CTGATCTGTCCCATCTGTGGGTTTACCTGCactcagaaaggtaacctcctccgccacattaaaCTG | 46L_47I_49P_50I_52G_53F_54T_55C_67L_ | 2.355690884 |
| 4212 | 7944 | CTCTGGTGCGAAATATGTGGTTTTTTTTGTactcagaaaggtaacctcctccgccacattaaaCTG | 46L_47W_49E_50I_52G_53F_54F_55C_67L_ | 2.353354738 |
| 4213 | 4492 | TTCTACTGTAATGTATGTGGGGCACCGTTTactcagaaaggtaacctcctccgccacattaaaCTG | 46F_47Y_49N_50V_52G_53A_54P_55F_67L_ | 2.350138169 |
| 4214 | 4491 | TTCTATTGTAACGTTTGTGGGGCAAACTTTactcagaaaggtaacctcctccgccacattaaaCTG | 46F_47Y_49N_50V_52G_53A_54N_55F_67L_ | 2.34917271 |
| 4215 | 2585 | TTCATGTGTCCAATTTGTGGGGCAGGATTTactcagaaaggtaacctcctccgccacattaaaCTG | 46F_47M_49P_50I_52G_53A_54G_55F_67L_ | 2.349025044 |
| 4216 | 6590 | CTCCATTGTCCGATTTGTGGCTTCATGTGTactcagaaaggtaacctcctccgccacattaaaCTG | 46L_47H_49P_50I_52G_53F_54M_55C_67L_ | 2.348457019 |
| 4217 | 4459 | TTCTATTGTAACATCTGTGGGGCATATTTTactcagaaaggtaacctcctccgccacattaaaCTG | 46F_47Y_49N_50I_52G_53A_54Y_55F_67L_ | 2.348345845 |
| 4218 | 3473 | TTCTCCTGTGAGGTGTGTGGGGCACAGTTTactcagaaaggtaacctcctccgccacattaaaCTG | 46F_47S_49E_50V_52G_53A_54Q_55F_67L_ | 2.348240024 |
| 4219 | 1788 | TTCATATGTGAGGTGTGTGGGGCAAAATTTactcagaaaggtaacctcctccgccacattaaaCTG | 46F_47I_49E_50V_52G_53A_54K_55F_67L_ | 2.346986459 |
| 4220 | 3705 | TTCACTTGTGAGGTGTGTGGGGCAGGTTTTactcagaaaggtaacctcctccgccacattaaaCTG | 46F_47T_49E_50V_52G_53A_54G_55F_67L_ | 2.344936647 |
| 4221 | 1199 | TTCTTTTGTCCCGTGTGTGGGGCATATTTTactcagaaaggtaacctcctccgccacattaaaCTG | 46F_47F_49P_50V_52G_53A_54Y_55F_67L_ | 2.344506926 |
| 4222 | 6478 | CTCGCCTGCCCGATTTGCGGGTTCTGGTGCactcagaaaggtaacctcctccgccacattaaaCTG | 46L_47A_49P_50I_52G_53F_54W_55C_67L_ | 2.339951164 |
| 4223 | 2887 | TTCCAGTGTGACATCTGTGGGGCAATCTTTactcagaaaggtaacctcctccgccacattaaaCTG | 46F_47Q_49D_50I_52G_53A_54I_55F_67L_ | 2.338711955 |
| 4224 | 1270 | TTCGGATGTGAGATCTGTGGGGCAATGTTTactcagaaaggtaacctcctccgccacattaaaCTG | 46F_47G_49E_50I_52G_53A_54M_55F_67L_ | 2.338690886 |
| 4225 | 6672 | CTGATGTGTCCTATTTGCGGCTTCCCGTGCactcagaaaggtaacctcctccgccacattaaaCTG | 46L_47M_49P_50I_52G_53F_54P_55C_67L_ | 2.336815562 |
| 4226 | 1420 | TTCGGTTGTCCGGTCTGTGGGGCAGCCTTTactcagaaaggtaacctcctccgccacattaaaCTG | 46F_47G_49P_50V_52G_53A_54A_55F_67L_ | 2.335812749 |
| 4227 | 4269 | TTCTGGTGTCCCATTTGTGGGGCACTGTTTactcagaaaggtaacctcctccgccacattaaaCTG | 46F_47W_49P_50I_52G_53A_54L_55F_67L_ | 2.333636942 |
| 4228 | 193 | TTCGCCTGTCCCATCTGTGGGGCACAATTTactcagaaaggtaacctcctccgccacattaaaCTG | 46F_47A_49P_50I_52G_53A_540_55F_67L_ | 2.332132949 |
| 4229 | 1508 | TTCCACTGTGAGATTTGTGGGGCAAAGTTTactcagaaaggtaacctcctccgccacattaaaCTG | 46F_47H_49E_50I_52G_53A_54K_55F_67L_ | 2.327842311 |
| 4230 | 4288 | TTCTGGTGTCCACAGTGTGGGGCAAAGTTTactcagaaaggtaacctcctccgccacattaaaCTG | 46F_47W_49P_50Q_52G_53A_54K_55F_67L_ | 2.326797303 |
| 4231 | 1064 | TTCTTCTGTGAGGTGTGTGGGGCATTCTTTactcagaaaggtaacctcctccgccacattaaaCTG | 46F_47F_49E_50V_52G_53A_54F_55F_67L_ | 2.326548464 |
| 4232 | 7634 | CTATGTTGCGAGATCTGTGGCTTCCGCTGTactcagaaaggtaacctcctccgccacattaaaCTG | 46L_47C_49E_50I_52G_53F_54R_55C_67L_ | 2.325349511 |
| 4233 | 4450 | TTCTATTGTAACATCTGTGGGGCAATGTTTactcagaaaggtaacctcctccgccacattaaaCTG | 46F_47Y_49N_50I_52G_53A_54M_55F_67L_ | 2.323228649 |
| 4234 | 1874 | TTCATCTGTCCCATTTGTGGGGCACGATTTactcagaaaggtaacctcctccgccacattaaaCTG | 46F_47I_49P_50I_52G_53A_54R_55F_67L_ | 2.323136568 |
| 4235 | 4092 | TTCTGGTGTGATATCTGTGGGGCACCCTTTactcagaaaggtaacctcctccgccacattaaaCTG | 46F_47W_49D_50I_52G_53A_54P_55F_67L_ | 2.322156812 |
| 4236 | 4204 | TTCTGGTGTAACATATGTGGGGCATTTTTTactcagaaaggtaacctcctccgccacattaaaCTG | 46F_47W_49N_50I_52G_53A_54F_55F_67L_ | 2.320211048 |
| 4237 | 1096 | TTCTTCTGTAATATTTGTGGGGCAACCTTTactcagaaaggtaacctcctccgccacattaaaCTG | 46F_47F_49N_50I_52G_53A_54T_55F_67L_ | 2.317071361 |
| 4238 | 4113 | TTCTGGTGTGATCAATGTGGGGCACAGTTTactcagaaaggtaacctcctccgccacattaaaCTG | 46F_47W_49D_50Q_52G_53A_54Q_55F_67L_ | 2.315097388 |
| 4239 | 3836 | TTCACGTGTCCCGTCTGTGGGGCAACCTTTactcagaaaggtaacctcctccgccacattaaaCTG | 46F_47T_49P_50V_52G_53A_54T_55F_67L_ | 2.313764824 |
| 4240 | 3797 | TTCACCTGTCCTATTTGTGGGGCAGTGTTTactcagaaaggtaacctcctccgccacattaaaCTG | 46F_47T_49P_50I_52G_53A_54V_55F_67L_ | 2.310456625 |
| 4241 | 6573 | CTGGGCTGTCCAATCTGCGGGTTTCAATGCactcagaaaggtaacctcctccgccacattaaaCTG | 46L_47G_49P_50I_52G_53F_54Q_55C_67L_ | 2.308354213 |
| 4242 | 4023 | TTCGTGTGTCCCATCTGTGGGGCAGAATTTactcagaaaggtaacctcctccgccacattaaaCTG | 46F_47V_49P_50I_52G_53A_54E_55F_67L_ | 2.305773654 |
| 4243 | 7600 | CTTGCCTGCGAGATATGCGGCTTTGCCTGTactcagaaaggtaacctcctccgccacattaaaCTG | 46L_47A_49E_50I_52G_53F_54A_55C_67L_ | 2.304917249 |
| 4244 | 2897 | TTCCAGTGTGATATTTGTGGGGCAGTATTTactcagaaaggtaacctcctccgccacattaaaCTG | 46F_47Q_49D_50I_52G_53A_54V_55F_67L_ | 2.3034176 |
| 4245 | 1190 | TTCTTTTGTCCCGTGTGTGGGGCAATGTTTactcagaaaggtaacctcctccgccacattaaaCTG | 46F_47F_49P_50V_52G_53A_54M_55F_67L_ | 2.302697024 |
| 4246 | 3374 | TTCTCTTGTGATATCTGTGGGGCACGATTTactcagaaaggtaacctcctccgccacattaaaCTG | 46F_47S_49D_50I_52G_53A_54R_55F_67L_ | 2.302350459 |
| 4247 | 4309 | TTCTGGTGTCCAGTATGTGGGGCACTGTTTactcagaaaggtaacctcctccgccacattaaaCTG | 46F_47W_49P_50V_52G_53A_54L_55F_67L_ | 2.30212856 |
| 4248 | 7753 | CTAATCTGTGAGATATGCGGCTTTCAGTGCactcagaaaggtaacctcctccgccacattaaaCTG | 46L_47I_49E_50I_52G_53F_54Q_55C_67L_ | 2.299807465 |
| 4249 | 3720 | TTCACTTGTAATATCTGTGGGGCAGCCTTTactcagaaaggtaacctcctccgccacattaaaCTG | 46F_47T_49N_50I_52G_53A_54A_55F_67L_ | 2.296348104 |
| 4250 | 1560 | TTCCACTGTAATATTTGTGGGGCAGCCTTTactcagaaaggtaacctcctccgccacattaaaCTG | 46F_47H_49N_50I_52G_53A_54A_55F_67L_ | 2.296236842 |
| 4251 | 6499 | CTATGCTGTCCTATTTGTGGTTTCTACTGTactcagaaaggtaacctcctccgccacattaaaCTG | 46L_47C_49P_50I_52G_53F_54Y_55C_67L_ | 2.294968578 |
| 4252 | 3615 | TTCACATGTGATATATGTGGGGCAAGCTTTactcagaaaggtaacctcctccgccacattaaaCTG | 46F_47T_49D_50I_52G_53A_54S_55F_67L_ | 2.294920296 |
| 4253 | 6783 | CTAGTGTGTCCGATCTGTGGCTTTGAATGTactcagaaaggtaacctcctccgccacattaaaCTG | 46L_47V_49P_50I_52G_53F_54E_55C_67L_ | 2.290350604 |
| 4254 | 1680 | TTCATCTGTGATATCTGTGGGGCAGCCTTTactcagaaaggtaacctcctccgccacattaaaCTG | 46F_47I_49D_50I_52G_53A_54A_55F_67L_ | 2.289806468 |
| 4255 | 1395 | TTCGGCTGTCCCATCTGTGGGGCATCATTTactcagaaaggtaacctcctccgccacattaaaCTG | 46F_47G_49P_50I_52G_53A_54S_55F_67L_ | 2.283331717 |
| 4256 | 4099 | TTCTGGTGTGACATCTGTGGGGCATACTTTactcagaaaggtaacctcctccgccacattaaaCTG | 46F_47W_49D_50I_52G_53A_54Y_55F_67L_ | 2.281071671 |
| 4257 | 3915 | TTCGTGTGTGAAATCTGTGGGGCAAGCTTTactcagaaaggtaacctcctccgccacattaaaCTG | 46F_47V_49E_50I_52G_53A_54S_55F_67L_ | 2.278788734 |
| 4258 | 3000 | TTCCAGTGTAATATTTGTGGGGCAGCATTTactcagaaaggtaacctcctccgccacattaaaCTG | 46F_47Q_49N_50I_52G_53A_54A_55F_67L_ | 2.277905268 |
| 4259 | 1394 | TTCGGGTGTCCAATTTGTGGGGCACGCTTTactcagaaaggtaacctcctccgccacattaaaCTG | 46F_47G_49P_50I_52G_53A_54R_55F_67L_ | 2.277743822 |
| 4260 | 234 | TTCGCCTGTCCTGTCTGTGGGGCACGGTTTactcagaaaggtaacctcctccgccacattaaaCTG | 46F_47A_49P_50V_52G_53A_54R_55F_67L_ | 2.277493039 |
| 4261 | 3461 | TTCAGCTGTGAAGTCTGTGGGGCATGCTTTactcagaaaggtaacctcctccgccacattaaaCTG | 46F_47S_49E_50V_52G_53A_54C_55F_67L_ | 2.275370368 |
| 4262 | 3676 | TTCACTTGTGAAATTTGTGGGGCAACCTTTactcagaaaggtaacctcctccgccacattaaaCTG | 46F_47T_49E_50I_52G_53A_54T_55F_67L_ | 2.274818316 |
| 4263 | 1315 | TTCGGCTGTGAAGTTTGTGGGGCAAGTTTTactcagaaaggtaacctcctccgccacattaaaCTG | 46F_47G_49E_50V_52G_53A_54S_55F_67L_ | 2.273922327 |
| 4264 | 4401 | TTCTATTGTGAACAGTGTGGGGCATGTTTTactcagaaaggtaacctcctccgccacattaaaCTG | 46F_47Y_49E_50Q_52G_53A_54C_55F_67L_ | 2.267968281 |
| 4265 | 341 | TTCTGCTGTGAAGTATGTGGGGCATGTTTTactcagaaaggtaacctcctccgccacattaaaCTG | 46F_47C_49E_50V_52G_53A_54C_55F_67L_ | 2.26717582 |
| 4266 | 7733 | TTACACTGCGAGATTTGTGGCTTCCAATGCactcagaaaggtaacctcctccgccacattaaaCTG | 46L_47H_49E_50I_52G_53F_54Q_55C_67L_ | 2.266333481 |
| 4267 | 3114 | TTCCAGTGTCCCGTATGTGGGGCAAGGTTTactcagaaaggtaacctcctccgccacattaaaCTG | 46F_47Q_49P_50V_52G_53A_54R_55F_67L_ | 2.266045821 |
| 4268 | 7754 | CTGATCTGTGAAATTTGTGGATTCCGGTGCactcagaaaggtaacctcctccgccacattaaaCTG | 46L_47I_49E_50I_52G_53F_54R_55C_67L_ | 2.263989479 |
| 4269 | 3300 | TTCCGCTGTCCAATCTGTGGGGCAGCCTTTactcagaaaggtaacctcctccgccacattaaaCTG | 46F_47R_49P_50I_52G_53A_54A_55F_67L_ | 2.263038632 |
| 4270 | 1274 | TTCGGCTGTGAGATTTGTGGGGCAAGATTTactcagaaaggtaacctcctccgccacattaaaCTG | 46F_47G_49E_50I_52G_53A_54R_55F_67L_ | 2.262745063 |
| 4271 | 1016 | TTCTTCTGTGATGTTTGTGGGGCAACTTTTactcagaaaggtaacctcctccgccacattaaaCTG | 46F_47F_49D_50V_52G_53A_54T_55F_67L_ | 2.262728942 |
| 4272 | 3110 | TTCCAGTGTCCAGTCTGTGGGGCAATGTTTactcagaaaggtaacctcctccgccacattaaaCTG | 46F_47Q_49P_50V_52G_53A_54M_55F_67L_ | 2.262090983 |
| 4273 | 3492 | TTCTCATGTAATATCTGTGGGGCACCATTTactcagaaaggtaacctcctccgccacattaaaCTG | 46F_47S_49N_50I_52G_53A_54P_55F_67L_ | 2.259894364 |
| 4274 | 1541 | TTCCATTGTGAGGTGTGTGGGGCATGCTTTactcagaaaggtaacctcctccgccacattaaaCTG | 46F_47H_49E_50V_52G_53A_54C_55F_67L_ | 2.25409135 |
| 4275 | 3799 | TTCACTTGTCCAATCTGTGGGGCATATTTTactcagaaaggtaacctcctccgccacattaaaCTG | 46F_47T_49P_50I_52G_53A_54Y_55F_67L_ | 2.253504342 |
| 4276 | 132 | TTCGCCTGTAACATCTGTGGGGCACCCTTTactcagaaaggtaacctcctccgccacattaaaCTG | 46F_47A_49N_50I_52G_53A_54P_55F_67L_ | 2.25238214 |
| 4277 | 316 | TTCTGCTGTGAGATCTGTGGGGCAACCTTTactcagaaaggtaacctcctccgccacattaaaCTG | 46F_47C_49E_50I_52G_53A_54T_55F_67L_ | 2.249956004 |
| 4278 | 6701 | CTCCAATGCCCCATTTGTGGGTTCTGCTGTactcagaaaggtaacctcctccgccacattaaaCTG | 46L_47Q_49P_50I_52G_53F_54C_55C_67L_ | 2.2475271 |
| 4279 | 473 | TTCTGCTGTCCCGTTTGTGGGGCACAATTTactcagaaaggtaacctcctccgccacattaaaCTG | 46F_47C_49P_50V_52G_53A_54Q_55F_67L_ | 2.246995331 |
| 4280 | 2888 | TTCCAGTGTGATATCTGTGGGGCAAAGTTTactcagaaaggtaacctcctccgccacattaaaCTG | 46F_47Q_49D_50I_52G_53A_54K_55F_67L_ | 2.246627012 |
| 4281 | 3553 | TTCTCCTGTCCGATATGTGGGGCACAGTTTactcagaaaggtaacctcctccgccacattaaaCTG | 46F_47S_49P_50I_52G_53A_54Q_55F_67L_ | 2.246183938 |
| 4282 | 3480 | TTCTCCTGTAACATCTGTGGGGCAGCATTTactcagaaaggtaacctcctccgccacattaaaCTG | 46F_47S_49N_50I_52G_53A_54A_55F_67L_ | 2.246086735 |
| 4283 | 4248 | TTCTGGTGTAACGTGTGTGGGGCAAAGTTTactcagaaaggtaacctcctccgccacattaaaCTG | 46F_47W_49N_50V_52G_53A_54K_55F_67L_ | 2.246019264 |
| 4284 | 2112 | TTCAAGTGTCCTATCTGTGGGGCACCTTTTactcagaaaggtaacctcctccgccacattaaaCTG | 46F_47K_49P_50I_52G_53A_54P_55F_67L_ | 2.243311131 |
| 4285 | 1300 | TTCGGGTGTGAGGTCTGTGGGGCAGCTTTTactcagaaaggtaacctcctccgccacattaaaCTG | 46F_47G_49E_50V_52G_53A_54A_55F_67L_ | 2.243036776 |
| 4286 | 6614 | CTGATTTGCCCTATCTGTGGCTTTCGGTGTactcagaaaggtaacctcctccgccacattaaaCTG | 46L_47I_49P_50I_52G_53F_54R_55C_67L_ | 2.240665982 |
| 4287 | 3595 | TTCAGCTGTCCCGTTTGTGGGGCATCCTTTactcagaaaggtaacctcctccgccacattaaaCTG | 46F_47S_49P_50V_52G_53A_54S_55F_67L_ | 2.239569436 |
| 4288 | 2953 | TTCCAGTGTGAGATTTGTGGGGCACAGTTTactcagaaaggtaacctcctccgccacattaaaCTG | 46F_47Q_49E_50I_52G_53A_54Q_55F_67L_ | 2.23811841 |
| 4289 | 7756 | CTCATCTGCGAGATCTGTGGATTCACCTGTactcagaaaggtaacctcctccgccacattaaaCTG | 46L_47I_49E_50I_52G_53F_54T_55C_67L_ | 2.237755508 |
| 4290 | 4281 | TTCTGGTGTCCCCAGTGTGGGGCATGCTTTactcagaaaggtaacctcctccgccacattaaaCTG | 46F_47W_49P_50Q_52G_53A_54C_55F_67L_ | 2.237256513 |
| 4291 | 7740 | CTTATCTGCGAGATTTGTGGATTTGCGTGTactcagaaaggtaacctcctccgccacattaaaCTG | 46L_47I_49E_50I_52G_53F_54A_55C_67L_ | 2.236977053 |
| 4292 | 2950 | TTCCAGTGTGAGATCTGTGGGGCAATGTTTactcagaaaggtaacctcctccgccacattaaaCTG | 46F_47Q_49E_50I_52G_53A_54M_55F_67L_ | 2.236265433 |
| 4293 | 254 | TTCTGTTGTGATATCTGTGGGGCACGCTTTactcagaaaggtaacctcctccgccacattaaaCTG | 46F_47C_49D_50I_52G_53A_54R_55F_67L_ | 2.233006706 |
| 4294 | 353 | TTCTGCTGTGAGGTGTGTGGGGCACAGTTTactcagaaaggtaacctcctccgccacattaaaCTG | 46F_47C_49E_50V_52G_53A_54Q_55F_67L_ | 2.232697818 |
| 4295 | 4303 | TTCTGGTGTCCAGTCTGTGGGGCAGAATTTactcagaaaggtaacctcctccgccacattaaaCTG | 46F_47W_49P_50V_52G_53A_54E_55F_67L_ | 2.231290552 |
| 4296 | 4089 | TTCTGGTGTGATATCTGTGGGGCATTATTTactcagaaaggtaacctcctccgccacattaaaCTG | 46F_47W_49D_50I_52G_53A_54L_55F_67L_ | 2.229887644 |
| 4297 | 1674 | TTCCACTGTCCCGTGTGTGGGGCACGCTTTactcagaaaggtaacctcctccgccacattaaaCTG | 46F_47H_49P_50V_52G_53A_54R_55F_67L_ | 2.22753964 |
| 4298 | 421 | TTCTGTTGTCCAATTTGTGGGGCATGCTTTactcagaaaggtaacctcctccgccacattaaaCTG | 46F_47C_49P_50I_52G_53A_54C_55F_67L_ | 2.225444638 |
| 4299 | 1184 | TTCTTCTGTCCGGTGTGTGGGGCATTTTTTactcagaaaggtaacctcctccgccacattaaaCTG | 46F_47F_49P_50V_52G_53A_54F_55F_67L_ | 2.222137125 |
| 4300 | 3541 | TTCTCATGTCCTATATGTGGGGCATGTTTTactcagaaaggtaacctcctccgccacattaaaCTG | 46F_47S_49P_50I_52G_53A_54C_55F_67L_ | 2.220594284 |
| 4301 | 3845 | TTCGTGTGTGACATTTGTGGGGCAGGATTTactcagaaaggtaacctcctccgccacattaaaCTG | 46F_47V_49D_50I_52G_53A_54G_55F_67L_ | 2.217786868 |
| 4302 | 6707 | TTGCAGTGCCCAATTTGCGGTTTCATTTGTactcagaaaggtaacctcctccgccacattaaaCTG | 46L_47Q_49P_50I_52G_53F_54I_55C_67L_ | 2.216291132 |
| 4303 | 6461 | CTTGCTTGCCCAATTTGCGGGTTTTGTTGCactcagaaaggtaacctcctccgccacattaaaCTG | 46L_47A_49P_50I_52G_53F_54C_55C_67L_ | 2.21515586 |
| 4304 | 252 | TTCTGTTGTGATATCTGTGGGGCACCTTTTactcagaaaggtaacctcctccgccacattaaaCTG | 46F_47C_49D_50I_52G_53A_54P_55F_67L_ | 2.214751686 |
| 4305 | 7691 | CTATTCTGCGAGATCTGTGGGTTCAATTGCactcagaaaggtaacctcctccgccacattaaaCTG | 46L_47F_49E_50I_52G_53F_54N_55C_67L_ | 2.210919072 |
| 4306 | 2620 | TTCATGTGTCCCGTGTGTGGGGCAGCTTTTactcagaaaggtaacctcctccgccacattaaaCTG | 46F_47M_49P_50V_52G_53A_54A_55F_67L_ | 2.209329743 |
| 4307 | 1390 | TTCGGCTGTCCCATTTGTGGGGCAATGTTTactcagaaaggtaacctcctccgccacattaaaCTG | 46F_47G_49P_50I_52G_53A_54M_55F_67L_ | 2.191207784 |
| 4308 | 4543 | TTCTATTGTCCCGTGTGTGGGGCAGAGTTTactcagaaaggtaacctcctccgccacattaaaCTG | 46F_47Y_49P_50V_52G_53A_54E_55F_67L_ | 2.191064591 |
| 4309 | 3785 | TTCACTTGTCCCATCTGTGGGGCAGGCTTTactcagaaaggtaacctcctccgccacattaaaCTG | 46F_47T_49P_50I_52G_53A_54G_55F_67L_ | 2.190883654 |
| 4310 | 3360 | TTCAGCTGTGATATTTGTGGGGCAGCCTTTactcagaaaggtaacctcctccgccacattaaaCTG | 46F_47S_49D_50I_52G_53A_54A_55F_67L_ | 2.190793254 |
| 4311 | 4448 | TTCTACTGTAATATCTGTGGGGCAAAGTTTactcagaaaggtaacctcctccgccacattaaaCTG | 46F_47Y_49N_50I_52G_53A_54K_55F_67L_ | 2.189214468 |
| 4312 | 1693 | TTCATTTGTGATATATGTGGGGCACAGTTTactcagaaaggtaacctcctccgccacattaaaCTG | 46F_47I_49D_50I_52G_53A_54Q_55F_67L_ | 2.186555995 |
| 4313 | 3713 | TTCACCTGTGAGGTGTGTGGGGCACAATTTactcagaaaggtaacctcctccgccacattaaaCTG | 46F_47T_49E_50V_52G_53A_54Q_55F_67L_ | 2.186518375 |
| 4314 | 3665 | TTCACGTGTGAGATATGTGGGGCAGGATTTactcagaaaggtaacctcctccgccacattaaaCTG | 46F_47T_49E_50I_52G_53A_54G_55F_67L_ | 2.185577198 |
| 4315 | 6753 | CTCTCATGCCCAATTTGTGGATTCCAGTGCactcagaaaggtaacctcctccgccacattaaaCTG | 46L_47S_49P_50I_52G_53F_54Q_55C_67L_ | 2.184426934 |
| 4316 | 7683 | CTGTTTTGCGAGATCTGCGGGTTCGAGTGTactcagaaaggtaacctcctccgccacattaaaCTG | 46L_47F_49E_50I_52G_53F_54E_55C_67L_ | 2.18186575 |
| 4317 | 6588 | CTTCACTGTCCTATCTGTGGGTTCAAATGCactcagaaaggtaacctcctccgccacattaaaCTG | 46L_47H_49P_50I_52G_53F_54K_55C_67L_ | 2.179102603 |
| 4318 | 3071 | TTCCAGTGTCCTATTTGTGGGGCAAATTTTactcagaaaggtaacctcctccgccacattaaaCTG | 46F_47Q_49P_50I_52G_53A_54N_55F_67L_ | 2.178701118 |
| 4319 | 6809 | CTTTGGTGCCCCATCTGTGGTTTTCTTTGTactcagaaaggtaacctcctccgccacattaaaCTG | 46L_47W_49P_50I_52G_53F_54L_55C_67L_ | 2.176250481 |
| 4320 | 4074 | TTCGTCTGTCCAGTGTGTGGGGCAAGGTTTactcagaaaggtaacctcctccgccacattaaaCTG | 46F_47V_49P_50V_52G_53A_54R_55F_67L_ | 2.175217902 |
| 4321 | 4084 | TTCTGGTGTGATATTTGTGGGGCATTCTTTactcagaaaggtaacctcctccgccacattaaaCTG | 46F_47W_49D_50I_52G_53A_54F_55F_67L_ | 2.17472827 |
| 4322 | 1863 | TTCATATGTCCGATCTGTGGGGCAGAGTTTactcagaaaggtaacctcctcogccacattaaaCTG | 46F_47I_49P_50I_52G_53A_54E_55F_67L_ | 2.171588171 |
| 4323 | 240 | TTCTGCTGTGACATTTGTGGGGCAGCATTTactcagaaaggtaacctcctccgccacattaaaCTG | 46F_47C_49D_50I_52G_53A_54A_55F_67L_ | 2.17076523 |
| 4324 | 4111 | TTCTGGTGTGACCAATGTGGGGCAAACTTTactcagaaaggtaacctcctccgccacattaaaCTG | 46F_47W_49D_50Q_52G_53A_54N_55F_67L_ | 2.167801298 |
| 4325 | 101 | TTCGCATGTGAGGTGTGTGGGGCATGCTTTactcagaaaggtaacctcctccgccacattaaaCTG | 46F_47A_49E_50V_52G_53A_54C_55F_67L_ | 2.160070025 |
| 4326 | 1145 | TTCTTTTGTCCCATCTGTGGGGCAGGATTTactcagaaaggtaacctcctccgccacattaaaCTG | 46F_47F_49P_50I_52G_53A_54G_55F_67L_ | 2.157952735 |
| 4327 | 7963 | TTGTATTGCGAAATTTGCGGCTTTGAGTGCactcagaaaggtaacctcctccgccacattaaaCTG | 46L_47Y_49E_50I_52G_53F_54E_55C_67L_ | 2.157735742 |
| 4328 | 4207 | TTCTGGTGTAATATTTGTGGGGCAATATTTactcagaaaggtaacctcctccgccacattaaaCTG | 46F_47W_49N_50I_52G_53A_54I_55F_67L_ | 2.156511312 |
| 4329 | 6717 | CTGCAGTGCCCTATCTGCGGCTTCGTTTGTactcagaaaggtaacctcctccgccacattaaaCTG | 46L_47Q_49P_50I_52G_53F_54V_55C_67L_ | 2.156038568 |
| 4330 | 431 | TTCTGTTGTCCCATCTGTGGGGCAAATTTTactcagaaaggtaacctcctccgccacattaaaCTG | 46F_47C_49P_50I_52G_53A_54N_55F_67L_ | 2.155589491 |
| 4331 | 7965 | CTATACTGTGAAATCTGCGGATTCGGGTGCactcagaaaggtaacctcctccgccacattaaaCTG | 46L_47Y_49E_50I_52G_53F_54G_55C_67L_ | 2.154986628 |
| 4332 | 6564 | TTAGGCTGTCCGATTTGTGGCTTCTTTTGCactcagaaaggtaacctcctccgccacattaaaCTG | 46L_47G_49P_50I_52G_53F_54F_55C_67L_ | 2.154623274 |
| 4333 | 6838 | CTGTACTGCCCTATTTGTGGATTTTGGTGCactcagaaaggtaacctcctccgccacattaaaCTG | 46L_47Y_49P_50I_52G_53F_54W_55C_67L_ | 2.153785566 |
| 4334 | 2172 | TTCCTGTGTGATATCTGTGGGGCACCATTTactcagaaaggtaacctcctccgccacattaaaCTG | 46F_47L_49D_50I_52G_53A_54P_55F_67L_ | 2.150714673 |
| 4335 | 235 | TTCGCCTGTCCCGTGTGTGGGGCAAGCTTTactcagaaaggtaacctcctccgccacattaaaCTG | 46F_47A_49P_50V_52G_53A_54S_55F_67L_ | 2.150310111 |
| 4336 | 6710 | CTGCAATGTCCAATCTGTGGCTTTATGTGCactcagaaaggtaacctcctccgccacattaaaCTG | 46L_47Q_49P_50I_52G_53F_54M_55C_67L_ | 2.149165729 |
| 4337 | 6790 | CTTGTATGTCCAATCTGCGGCTTTATGTGCactcagaaaggtaacctcctccgccacattaaaCTG | 46L_47V_49P_50I_52G_53F_54M_55C_67L_ | 2.149153613 |
| 4338 | 4535 | TTCTATTGTCCGCAGTGTGGGGCATCCTTTactcagaaaggtaacctcctccgccacattaaaCTG | 46F_47Y_49P_50Q_52G_53A_54S_55F_67L_ | 2.146073031 |
| 4339 | 1866 | TTCATTTGTCCTATATGTGGGGCACACTTTactcagaaaggtaacctcctccgccacattaaaCTG | 46F_47I_49P_50I_52G_53A_54H_55F_67L_ | 2.145894092 |
| 4340 | 2625 | TTCATGTGTCCAGTTTGTGGGGCAGGCTTTactcagaaaggtaacctcctccgccacattaaaCTG | 46F_47M_49P_50V_52G_53A_54G_55F_67L_ | 2.140211706 |
| 4341 | 6720 | CTCAGATGTCCTATATGTGGTTTCGCATGCactcagaaaggtaacctcctccgccacattaaaCTG | 46L_47R_49P_50I_52G_53F_54A_55C_67L_ | 2.139720291 |
| 4342 | 3556 | TTCTCATGTCCCATCTGTGGGGCAACTTTTactcagaaaggtaacctcctccgccacattaaaCTG | 46F_47S_49P_50I_52G_53A_54T_55F_67L_ | 2.137570823 |
| 4343 | 6725 | TTGCGCTGTCCCATATGCGGCTTCGGCTGCactcagaaaggtaacctcctccgccacattaaaCTG | 46L_47R_49P_50I_52G_53F_54G_55C_67L_ | 2.13732607 |
| 4344 | 4521 | TTCTACTGTCCTCAGTGTGGGGCATGCTTTactcagaaaggtaacctcctccgccacattaaaCTG | 46F_47Y_49P_50Q_52G_53A_54C_55F_67L_ | 2.135961679 |
| 4345 | 7815 | TTGATGTGTGAAATTTGCGGCTTTAGCTGCactcagaaaggtaacctcctccgccacattaaaCTG | 46L_47M_49E_50I_52G_53F_54S_55C_67L_ | 2.135133121 |
| 4346 | 6788 | CTGGTCTGCCCCATATGTGGTTTTAAGTGCactcagaaaggtaacctcctccgccacattaaaCTG | 46L_47V_49P_50I_52G_53F_54K_55C_67L_ | 2.131096808 |
| 4347 | 4021 | TTCGTGTGTCCCATCTGTGGGGCATGTTTTactcagaaaggtaacctcctccgccacattaaaCTG | 46F_47V_49P_50I_52G_53A_54C_55F_67L_ | 2.128618651 |
| 4348 | 4325 | TTCTATTGTGATATCTGTGGGGCAGGGTTTactcagaaaggtaacctcctccgccacattaaaCTG | 46F_47Y_49D_50I_52G_53A_54G_55F_67L_ | 2.125758368 |
| 4349 | 1275 | TTCGGATGTGAGATCTGTGGGGCATCCTTTactcagaaaggtaacctcctccgccacattaaaCTG | 46F_47G_49E_50I_52G_53A_54S_55F_67L_ | 2.123404283 |
| 4350 | 1427 | TTCGGATGTCCCGTCTGTGGGGCAATATTTactcagaaaggtaacctcctccgccacattaaaCTG | 46F_47G_49P_50V_52G_53A_54I_55F_67L_ | 2.123018962 |
| 4351 | 7720 | TTGCATTGCGAGATTTGTGGATTTGCCTGCactcagaaaggtaacctcctccgccacattaaaCTG | 46L_47H_49E_50I_52G_53F_54A_55C_67L_ | 2.118934257 |
| 4352 | 4026 | TTCGTGTGTCCTATTTGTGGGGCACACTTTactcagaaaggtaacctcctccgccacattaaaCTG | 46F_47V_49P_50I_52G_53A_54H_55F_67L_ | 2.118774987 |
| 4353 | 7684 | CTGTTTTGTGAAATCTGTGGCTTTTTCTGCactcagaaaggtaacctcctccgccacattaaaCTG | 46L_47F_49E_50I_52G_53F_54F_55C_67L_ | 2.117956907 |
| 4354 | 356 | TTCTGCTGTGAAGTGTGTGGGGCAACCTTTactcagaaaggtaacctcctccgccacattaaaCTG | 46F_47C_49E_50V_52G_53A_54T_55F_67L_ | 2.117267905 |
| 4355 | 2881 | TTCCAATGTGATATATGTGGGGCATGTTTTactcagaaaggtaacctcctccgccacattaaaCTG | 46F_47Q_49D_50I_52G_53A_54C_55F_67L_ | 2.117140985 |
| 4356 | 6173 | TTCTTTTGCCCAATCTGCGGGCGACAGTTTactcagaaaggtaacctoctccgccacattaaaCTG | 46F_47F_49P_50I_52G_53R_54Q_55F_67L_ | 2.113819352 |
| 4357 | 2990 | TTCCAGTGTGAGGTGTGTGGGGCAATGTTTactcagaaaggtaacctcctccgccacattaaaCTG | 46F_47Q_49E_50V_52G_53A_54M_55F_67L_ | 2.113208039 |
| 4358 | 2932 | TTCCAATGTGACGTGTGTGGGGCACCCTTTactcagaaaggtaacctcctccgccacattaaaCTG | 46F_47Q_49D_50V_52G_53A_54P_55F_67L_ | 2.109063984 |
| 4359 | 1434 | TTCGGGTGTCCGGTCTGTGGGGCAAGGTTTactcagaaaggtaacctcctccgccacattaaaCTG | 46F_47G_49P_50V_52G_53A_54R_55F_67L_ | 2.104505361 |
| 4360 | 7914 | CTCACCTGTGAAATTTGCGGCTTCCGGTGCactcagaaaggtaacctcctccgccacattaaaCTG | 46L_47T_49E_50I_52G_53F_54R_55C_67L_ | 2.102046992 |
| 4361 | 1781 | TTCATCTGTGAAGTGTGTGGGGCATGTTTTactcagaaaggtaacctcctccgccacattaaaCTG | 46F_47I_49E_50V_52G_53A_54C_55F_67L_ | 2.100958104 |
| 4362 | 6560 | CTGGGGTGCCCAATCTGCGGCTTTGCATGTactcagaaaggtaacctcctccgccacattaaaCTG | 46L_47G_49P_50I_52G_53F_54A_55C_67L_ | 2.100905501 |
| 4363 | 6481 | CTGTGCTGTCCGATTTGTGGATTCTGTTGTactcagaaaggtaacctcctccgccacattaaaCTG | 46L_47C_49P_50I_52G_53F_54C_55C_67L_ | 2.100776216 |
| 4364 | 2954 | TTCCAATGTGAGATCTGTGGGGCACGTTTTactcagaaaggtaacctcctccgccacattaaaCTG | 46F_47Q_49E_50I_52G_53A_54R_55F_67L_ | 2.096849368 |
| 4365 | 305 | TTCTGCTGTGAAATCTGTGGGGCAGGTTTTactcagaaaggtaacctcctccgccacattaaaCTG | 46F_47C_49E_50I_52G_53A_54G_55F_67L_ | 2.093088269 |
| 4366 | 1628 | TTCCACTGTCCAATTTGTGGGGCAAAATTTactcagaaaggtaacctcctccgccacattaaaCTG | 46F_47H_49P_50I_52G_53A_54K_55F_67L_ | 2.092423524 |
| 4367 | 1310 | TTCGGATGTGAGGTTTGTGGGGCAATGTTTactcagaaaggtaacctcctccgccacattaaaCTG | 46F_47G_49E_50V_52G_53A_54M_55F_67L_ | 2.092068823 |
| 4368 | 181 | TTCGCCTGTCCTATTTGTGGGGCATGCTTTactcagaaaggtaacctcctccgccacattaaaCTG | 46F_47A_49P_50I_52G_53A_54C_55F_67L_ | 2.089170074 |
| 4369 | 352 | TTCTGTTGTGAGGTTTGTGGGGCACCCTTTactcagaaaggtaacctcctcogccacattaaaCTG | 46F_47C_49E_50V_52G_53A_54P_55F_67L_ | 2.087913855 |
| 4370 | 3825 | TTCACCTGTCCGGTTTGTGGGGCAGGTTTTactcagaaaggtaacctcctccgccacattaaaCTG | 46F_47T_49P_50V_52G_53A_54G_55F_67L_ | 2.087294968 |
| 4371 | 1321 | TTCGGCTGTAATATATGTGGGGCATGCTTTactcagaaaggtaacctcctccgccacattaaaCTG | 46F_47G_49N_50I_52G_53A_54C_55F_67L_ | 2.085259446 |
| 4372 | 1914 | TTCATATGTCCAGTCTGTGGGGCACGGTTTactcagaaaggtaacctcctccgccacattaaaCTG | 46F_47I_49P_50V_52G_53A_54R_55F_67L_ | 2.085086946 |
| 4373 | 4400 | TTCTATTGTGAGCAGTGTGGGGCAGCCTTTactcagaaaggtaacctcctccgccacattaaaCTG | 46F_47Y_49E_50Q_52G_53A_54A_55F_67L_ | 2.083658319 |
| 4374 | 1430 | TTCGGATGTCCGGTTTGTGGGGCAATGTTTactcagaaaggtaacctcctccgccacattaaaCTG | 46F_47G_49P_50V_52G_53A_54M_55F_67L_ | 2.082487802 |
| 4375 | 3593 | TTCTCGTGTCCTGTTTGTGGGGCACAGTTTactcagaaaggtaacctcctccgccacattaaaCTG | 46F_47S_49P_50V_52G_53A_54Q_55F_67L_ | 2.080433217 |
| 4376 | 1187 | TTCTTTTGTCCTGTCTGTGGGGCAATCTTTactcagaaaggtaacctcctccgccacattaaaCTG | 46F_47F_49P_50V_52G_53A_54I_55F_67L_ | 2.074839976 |
| 4377 | 3007 | TTCCAGTGTAACATATGTGGGGCAATCTTTactcagaaaggtaacctcctccgccacattaaaCTG | 46F_47Q_49N_50I_52G_53A_54I_55F_67L_ | 2.073016287 |
| 4378 | 1748 | TTCATTTGTGAGATCTGTGGGGCAAAGTTTactcagaaaggtaacctcctccgccacattaaaCTG | 46F_47I_49E_50I_52G_53A_54K_55F_67L_ | 2.072787119 |
| 4379 | 4545 | TTCTACTGTCCCGTCTGTGGGGCAGGATTTactcagaaaggtaacctcctccgccacattaaaCTG | 46F_47Y_49P_50V_52G_53A_54G_55F_67L_ | 2.072525411 |
| 4380 | 4534 | TTCTATTGTCCGCAATGTGGGGCAAGGTTTactcagaaaggtaacctcctccgccacattaaaCTG | 46F_47Y_49P_50Q_52G_53A_54R_55F_67L_ | 2.066661541 |
| 4381 | 6705 | CTGCAGTGTCCTATCTGCGGTTTCGGCTGTactcagaaaggtaacctcctccgccacattaaaCTG | 46L_47Q_49P_50I_52G_53F_54G_55C_67L_ | 2.066617096 |
| 4382 | 4052 | TTCGTGTGTCCTCAGTGTGGGGCACCTTTTactcagaaaggtaacctcctccgccacattaaaCTG | 46F_47V_49P_50Q_52G_53A_54P_55F_67L_ | 2.063627035 |
| 4383 | 301 | TTCTGCTGTGAAATCTGTGGGGCATGTTTTactcagaaaggtaacctcctccgccacattaaaCTG | 46F_47C_49E_50I_52G_53A_54C_55F_67L_ | 2.062985032 |
| 4384 | 1901 | TTCATCTGTCCAGTATGTGGGGCATGCTTTactcagaaaggtaacctcctccgccacattaaaCTG | 46F_47I_49P_50V_52G_53A_54C_55F_67L_ | 2.061764833 |
| 4385 | 7613 | CTGGCTTGCGAGATATGCGGTTTTCAGTGTactcagaaaggtaacctcctccgccacattaaaCTG | 46L_47A_49E_50I_52G_53F_54Q_55C_67L_ | 2.06108798 |
| 4386 | 4257 | TTCTGGTGTAATGTGTGTGGGGCAGTCTTTactcagaaaggtaacctcctccgccacattaaaCTG | 46F_47W_49N_50V_52G_53A_54V_55F_67L_ | 2.059298657 |
| 4387 | 3855 | TTCGTCTGTGATATATGTGGGGCAAGTTTTactcagaaaggtaacctcctccgccacattaaaCTG | 46F_47V_49D_50I_52G_53A_54S_55F_67L_ | 2.058193779 |
| 4388 | 113 | TTCGCATGTGAGGTGTGTGGGGCACAGTTTactcagaaaggtaacctcctccgccacattaaaCTG | 46F_47A_49E_50V_52G_53A_54Q_55F_67L_ | 2.057347537 |
| 4389 | 137 | TTCGCCTGTAACATCTGTGGGGCAGTCTTTactcagaaaggtaacctcctccgccacattaaaCTG | 46F_47A_49N_50I_52G_53A_54V_55F_67L_ | 2.053846855 |
| 4390 | 3074 | TTCCAATGTCCAATTTGTGGGGCAAGGTTTactcagaaaggtaacctcctccgccacattaaaCTG | 46F_47Q_49P_50I_52G_53A_54R_55F_67L_ | 2.05253405 |
| 4391 | 428 | TTCTGTTGTCCAATTTGTGGGGCAAAATTTactcagaaaggtaacctcctccgccacattaaaCTG | 46F_47C_49P_50I_52G_53A_54K_55F_67L_ | 2.050760338 |
| 4392 | 3065 | TTCCAGTGTCCCATTTGTGGGGCAGGTTTTactcagaaaggtaacctcctccgccacattaaaCTG | 46F_47Q_49P_50I_52G_53A_54G_55F_67L_ | 2.050106931 |
| 4393 | 7880 | CTGTCCTGTGAGATTTGTGGTTTCGCGTGCactcagaaaggtaacctcctccgccacattaaaCTG | 46L_47S_49E_50I_52G_53F_54A_55C_67L_ | 2.046965921 |
| 4394 | 4166 | TTCTGGTGTGAACAGTGTGGGGCACATTTTactcagaaaggtaacctcctccgccacattaaaCTG | 46F_47W_49E_50Q_52G_53A_54H_55F_67L_ | 2.046791868 |
| 4395 | 76 | TTCGCCTGTGAAATCTGTGGGGCAACATTTactcagaaaggtaacctcctccgccacattaaaCTG | 46F_47A_49E_50I_52G_53A_54T_55F_67L_ | 2.044627527 |
| 4396 | 6591 | CTCCACTGTCCTATCTGCGGATTCAACTGTactcagaaaggtaacctcctccgccacattaaaCTG | 46L_47H_49P_50I_52G_53F_54N_55C_67L_ | 2.044292815 |
| 4397 | 6585 | CTACATTGTCCTATCTGCGGCTTTGGGTGTactcagaaaggtaacctcctccgccacattaaaCTG | 46L_47H_49P_50I_52G_53F_54G_55C_67L_ | 2.04105345 |
| 4398 | 2635 | TTCATGTGTCCGGTCTGTGGGGCATCATTTactcagaaaggtaacctcctccgccacattaaaCTG | 46F_47M_49P_50V_52G_53A_54S_55F_67L_ | 2.039230179 |
| 4399 | 1794 | TTCATCTGTGAAGTCTGTGGGGCACGCTTTactcagaaaggtaacctcctccgccacattaaaCTG | 46F_47I_49E_50V_52G_53A_54R_55F_67L_ | 2.038116098 |
| 4400 | 6574 | CTGGGCTGCCCCATCTGCGGTTTCCGTTGCactcagaaaggtaacctcctccgccacattaaaCTG | 46L_47G_49P_50I_52G_53F_54R_55C_67L_ | 2.037779672 |
| 4401 | 3788 | TTCACCTGTCCAATCTGTGGGGCAAAGTTTactcagaaaggtaacctcctccgccacattaaaCTG | 46F_47T_49P_50I_52G_53A_54K_55F_67L_ | 2.036875064 |
| 4402 | 0 | TTCGCCTGTGATATCTGTGGGGCAGCTTTTactcagaaaggtaacctcctccgccacattaaaCTG | 46F_47A_49D_50I_52G_53A_54A_55F_67L_ | 2.036810704 |
| 4403 | 117 | TTCGCCTGTGAGGTGTGTGGGGCAGTCTTTactcagaaaggtaacctcctccgccacattaaaCTG | 46F_47A_49E_50V_52G_53A_54V_55F_67L_ | 2.035070088 |
| 4404 | 6771 | CTCACCTGTCCCATTTGTGGCTTTAACTGTactcagaaaggtaacctcctccgccacattaaaCTG | 46L_47T_49P_50I_52G_53F_54N_55C_67L_ | 2.034995487 |
| 4405 | 1685 | TTCATTTGTGACATATGTGGGGCAGGCTTTactcagaaaggtaacctcctccgccacattaaaCTG | 46F_47I_49D_50I_52G_53A_54G_55F_67L_ | 2.033327628 |
| 4406 | 116 | TTCGCATGTGAGGTTTGTGGGGCAACCTTTactcagaaaggtaacctcctccgccacattaaaCTG | 46F_47A_49E_50V_52G_53A_54T_55F_67L_ | 2.032770194 |
| 4407 | 4203 | TTCTGGTGTAACATTTGTGGGGCAGAATTTactcagaaaggtaacctcctccgccacattaaaCTG | 46F_47W_49N_50I_52G_53A_54E_55F_67L_ | 2.032616946 |
| 4408 | 6561 | CTGGGCTGCCCCATCTGCGGTTTCTGCTGCactcagaaaggtaacctcctccgccacattaaaCTG | 46L_47G_49P_50I_52G_53F_54C_55C_67L_ | 2.032089896 |
| 4409 | 4398 | TTCTATTGTGAGATCTGTGGGGCATGGTTTactcagaaaggtaacctcctccgccacattaaaCTG | 46F_47Y_49E_50I_52G_53A_54W_55F_67L_ | 2.031922249 |
| 4410 | 135 | TTCGCTTGTAATATTTGTGGGGCATCCTTTactcagaaaggtaacctcctccgccacattaaaCTG | 46F_47A_49N_50I_52G_53A_54S_55F_67L_ | 2.030843857 |
| 4411 | 1879 | TTCATATGTCCAATCTGTGGGGCATACTTTactcagaaaggtaacctcctccgccacattaaaCTG | 46F_47I_49P_50I_52G_53A_54Y_55F_67L_ | 2.028969939 |
| 4412 | 3105 | TTCCAGTGTCCCGTGTGTGGGGCAGGCTTTactcagaaaggtaacctcctccgccacattaaaCTG | 46F_47Q_49P_50V_52G_53A_54G_55F_67L_ | 2.028853831 |
| 4413 | 3911 | TTCGTTTGTGAGATTTGTGGGGCAAATTTTactcagaaaggtaacctcctccgccacattaaaCTG | 46F_47V_49E_50I_52G_53A_54N_55F_67L_ | 2.025688761 |
| 4414 | 1185 | TTCTTCTGTCCTGTATGTGGGGCAGGTTTTactcagaaaggtaacctcctccgccacattaaaCTG | 46F_47F_49P_50V_52G_53A_54G_55F_67L_ | 2.025597332 |
| 4415 | 3225 | TTCAGATGTGAGGTCTGTGGGGCAGGCTTTactcagaaaggtaacctcctccgccacattaaaCTG | 46F_47R_49E_50V_52G_53A_54G_55F_67L_ | 2.024879504 |
| 4416 | 4505 | TTCTATTGTCCAATATGTGGGGCAGGTTTTactcagaaaggtaacctcctccgccacattaaaCTG | 46F_47Y_49P_50I_52G_53A_54G_55F_67L_ | 2.023445267 |
| 4417 | 6746 | CTATCTTGCCCTATATGCGGCTTTCACTGCactcagaaaggtaacctcctccgccacattaaaCTG | 46L_47S_49P_50I_52G_53F_54H_55C_67L_ | 2.022496397 |
| 4418 | 1861 | TTCATCTGTCCCATCTGTGGGGCATGTTTTactcagaaaggtaacctcctccgccacattaaaCTG | 46F_47I_49P_50I_52G_53A_54C_55F_67L_ | 2.018995977 |
| 4419 | 6578 | CTGGGGTGCCCAATCTGTGGCTTTTGGTGCactcagaaaggtaacctcctccgccacattaaaCTG | 46L_47G_49P_50I_52G_53F_54W_55C_67L_ | 2.017659917 |
| 4420 | 3111 | TTCCAATGTCCGGTTTGTGGGGCAAACTTTactcagaaaggtaacctcctccgccacattaaaCTG | 46F_47Q_49P_50V_52G_53A_54N_55F_67L_ | 2.017551391 |
| 4421 | 108 | TTCGCTTGTGAAGTGTGTGGGGCAAAATTTactcagaaaggtaacctcctccgccacattaaaCTG | 46F_47A_49E_50V_52G_53A_54K_55F_67L_ | 2.017239513 |
| 4422 | 6818 | CTGTGGTGTCCCATTTGTGGTTTCTGGTGCactcagaaaggtaacctcctccgccacattaaaCTG | 46L_47W_49P_50I_52G_53F_54W_55C_67L_ | 2.01587826 |
| 4423 | 1741 | TTCATCTGTGAGATTTGTGGGGCATGCTTTactcagaaaggtaacctcctccgccacattaaaCTG | 46F_47I_49E_50I_52G_53A_54C_55F_67L_ | 2.015829939 |
| 4424 | 6490 | CTATGCTGCCCAATATGCGGGTTTATGTGCactcagaaaggtaacctcctccgccacattaaaCTG | 46L_47C_49P_50I_52G_53F_54M_55C_67L_ | 2.015083611 |
| 4425 | 1575 | TTCCATTGTAACATTTGTGGGGCATCATTTactcagaaaggtaacctcctccgccacattaaaCTG | 46F_47H_49N_50I_52G_53A_54S_55F_67L_ | 2.014779945 |
| 4426 | 3472 | TTCTCTTGTGAAGTATGTGGGGCACCCTTTactcagaaaggtaacctcctccgccacattaaaCTG | 46F_47S_49E_50V_52G_53A_54P_55F_67L_ | 2.013210958 |
| 4427 | 2505 | TTCATGTGTGAGGTGTGTGGGGCAGGATTTactcagaaaggtaacctcctccgccacattaaaCTG | 46F_47M_49E_50V_52G_53A_54G_55F_67L_ | 2.0122724 |
| 4428 | 3052 | TTCCAGTGTAACGTGTGTGGGGCACCATTTactcagaaaggtaacctcctccgccacattaaaCTG | 46F_47Q_49N_50V_52G_53A_54P_55F_67L_ | 2.011866965 |
| 4429 | 4480 | TTCTATTGTAATGTTTGTGGGGCAGCCTTTactcagaaaggtaacctcctccgccacattaaaCTG | 46F_47Y_49N_50V_52G_53A_54A_55F_67L_ | 2.01183574 |
| 4430 | 3017 | TTCCAATGTAACATATGTGGGGCAGTGTTTactcagaaaggtaacctcctccgccacattaaaCTG | 46F_47Q_49N_50I_52G_53A_54V_55F_67L_ | 2.010584662 |
| 4431 | 6695 | CTCAACTGCCCCATCTGCGGGTTTAGCTGCactcagaaaggtaacctcctccgccacattaaaCTG | 46L_47N_49P_50I_52G_53F_54S_55C_67L_ | 2.007693196 |
| 4432 | 6786 | CTGGTGTGCCCTATCTGTGGATTCCATTGCactcagaaaggtaacctcctccgccacattaaaCTG | 46L_47V_49P_50I_52G_53F_54H_55C_67L_ | 2.000848664 |

### Mutant Degron

As discussed herein, the system provided herein comprises a polypeptide and a compound as defined herein. Any of the polypeptides described herein can be comprised in the system. In some embodiments, also provided herein is a polypeptide as defined in the context of the system.

Accordingly, in some embodiments provided herein is a polypeptide comprising a variant of the amino acid sequence of SEQ ID NO: 1, wherein the variant comprises (i) a modification at the position corresponding to Q147 of SEQ ID NO: 2; and (ii) a modification at one or more of the positions corresponding to N149, Q150 and S154 of SEQ ID NO: 2.

As described in respect of the system herein, SEQ ID NO: 1 may correspond to amino acids F146-F155 of the polypeptide sequence of IKZF3 (SEQ ID NO: 2) or to amino acids F145-F154 of the polypeptide sequence of IKZF1 (SEQ ID NO: 3). The polypeptide may therefore alternatively comprise a variant of the amino acid sequence of SEQ ID NO: 1, wherein the variant comprises (i) a modification at the position corresponding to Q146 of SEQ ID NO: 3; and (ii) a modification at one or more of the positions corresponding to N148, Q149 and S153 of SEQ ID NO: 3. Those skilled in the art will appreciate that the variant may be a variant defined according SEQ ID NO: 3 and thereby comprise modifications at the positions which correspond to the positions of SEQ ID NO: 2 disclosed herein. The variant of SEQ ID NO: 1 may therefore be defined herein according SEQ ID NO: 2 or SEQ ID NO: 3.

The aforementioned modifications have been defined relative to SEQ ID NO: 2. Those skilled in the art will appreciate that the variant may be a variant of SEQ ID NO: 2 and comprise the aforementioned modifications. The variant may be a variant of SEQ ID NO: 3 and comprise the aforementioned modifications relative to SEQ ID NO: 3; for example, the variant may comprise a modification at one or more of the positions corresponding to N148, Q149 and S153 of SEQ ID NO: 3.

The polypeptide may be further defined according to any aspect of the polypeptide of the system described herein.

Provided herein is a polynucleotide encoding the polypeptide according to the invention.

The polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences, for example in an expression vector. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides can be synthesised according to methods well known in the art, as described by way of example in Sambrook et al. (1989, Molecular Cloning—a laboratory manual; Cold Spring Harbor Press). The nucleic acid molecules of the present invention may be provided in the form of an expression cassette which includes control sequences operably linked to the inserted sequence, thus allowing for expression of the polypeptide of the invention in vivo.

These expression cassettes, in turn, are typically provided within vectors (e.g., plasmids or recombinant viral vectors).

The polynucleotide may further encode a protein of interest in-frame relative to the encoded polypeptide.

Provided herein is a vector that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. The vector may also encode one or more fluorescent proteins, preferably at least two fluorescent proteins, and more preferably wherein one fluorescent protein is encoded in frame with a sequence encoding a degron polypeptide and target protein of interest such that the fluorescent protein is genetically fused to the degron or protein of interest, and wherein the other fluorescent protein is separated by an internal ribosomal entry site (IRES). Other suitable vectors would be apparent to persons skilled in the art. By way of further example in this regard we refer to Sambrook et al.

The invention also includes cells that have been modified to express a polypeptide of the invention. The cells may therefore comprise the polynucleotide of the invention and/or the vector of the invention. Such cells typically include prokaryotic cells such as bacteria, for example E. coli, or mammalian cells. Such bacteria or cells may be cultured using routine methods to produce a polypeptide of the invention.

### Compound

Also provided herein are compounds. Said compounds are useful in the systems and methods described herein in more detail.

In one aspect, the compound is a compound of Formula (B),

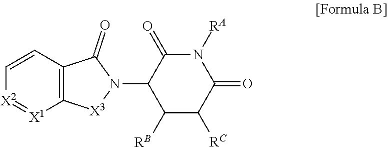

wherein
   X¹ is C—R¹ and X² is C—R² or N; or X¹ is C—R¹ or N and X² is C—R²;
   X³ is CHR^{D} or C═O;
   A:
      R¹ is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl; and
      R² is selected from N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
   or
   B:
      R¹ is selected from OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl; and
      R² is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, 5- to 10-membered heteroaryl, 5- to 10-membered heterocyclyl, C₅₋₁₀ carbocyclyl, OR²⁰, N(R²⁰)₂ and halogen; and wherein each aryl, heteroaryl, heterocyclyl and carbocyclyl group is independently unsubstituted or is substituted with 1, 2 or 3 substituents independently selected from R²⁰, OR²⁰, N(R²⁰)₂ and halogen;
   R^{A} is H or C₁0.4 alkyl which is unsubstituted or is substituted with 1, 2 or 3 substituents independently selected from OR²⁰, N(R²⁰)₂ and halogen;
   R^{B} is H or C₁₋₄ alkyl which is unsubstituted or is substituted with 1, 2 or 3 substituents independently selected from OR²⁰, N(R²⁰)₂ and halogen;
   R^{C} is H or C₁₋₄ alkyl which is unsubstituted or is substituted with 1, 2 or 3 substituents independently selected from OR²⁰, N(R²⁰)₂ and halogen;
   R^{D} is H or C₁₋₄ alkyl which is unsubstituted or is substituted with 1, 2 or 3 substituents independently selected from OR²⁰, N(R²⁰)₂ and halogen; and
   each R¹⁰, which may be the same or different, is independently H or C₁₋₄ alkyl which is unsubstituted or substituted with 1, 2 or 3 substituents independently selected from OR²⁰, N(R²⁰)₂ and halogen;
   each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl.

In one aspect, the compound is a compound of Formula (B),

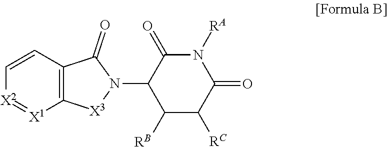

wherein
   X¹ is C—R¹ and X² is C—R² or N; or X¹ is C—R¹ or N and X² is C—R²;
   X³ is CHR^{D} or C═O;
   A:
      R¹ is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, C₅₋₁₀ heteroaryl, C₅₋₁₀ heterocyclyl, and C₅₋₁₀ carbocyclyl; and
      R² is selected from N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, C₅₋₁₀ heteroaryl, C₅₋₁₀ heterocyclyl, and C₅₋₁₀ carbocyclyl;
   or
   B:
      R¹ is selected from OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, C₅₋₁₀ heteroaryl, C₅₋₁₀ heterocyclyl, and C₅₋₁₀ carbocyclyl; and
      R² is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, C₅₋₁₀ heteroaryl, C₅₋₁₀ heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, C₅₋₁₀ heteroaryl, C₅₋₁₀ heterocyclyl, C₅₋₁₀ carbocyclyl, OR²⁰, N(R²⁰)₂ and halogen; and wherein each aryl, heteroaryl, heterocyclyl and carbocyclyl group is independently unsubstituted or is substituted with 1 or 2 substituents independently selected from R²⁰, OR²⁰, N(R²⁰)₂ and halogen;
   R^{A} is H or C₁0.4 alkyl which is unsubstituted or is substituted with 1, 2 or 3 substituents independently selected from OR²⁰, N(R²⁰)₂ and halogen;
   R^{B} is H or C₁0.4 alkyl which is unsubstituted or is substituted with 1, 2 or 3 substituents independently selected from OR²⁰, N(R²⁰)₂ and halogen;
   R^{C} is H or C₁0.4 alkyl which is unsubstituted or is substituted with 1, 2 or 3 substituents independently selected from OR²⁰, N(R²⁰)₂ and halogen;
   R^{D} is H or C₁0.4 alkyl which is unsubstituted or is substituted with 1, 2 or 3 substituents independently selected from OR²⁰, N(R²⁰)₂ and halogen; and
   each R¹⁰, which may be the same or different, is independently H or C₁0.4 alkyl which is unsubstituted or substituted with 1, 2 or 3 substituents independently selected from OR²⁰, N(R²⁰)₂ and halogen;
   each R²⁰, which may be the same or different, is independently H or methyl.
      Typically, the compound of Formula (B) is a compound of Formula (Ib),

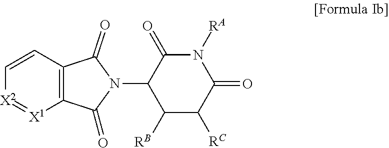

wherein
   X¹ is C—R¹ and X² is C—R² or N; or X¹ is C—R¹ or N and X² is C—R²;
   A:
      R¹ is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl; and
      R² is selected from N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl;
   or
   B:
      R¹ is selected from OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl; and
      R² is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, C₅₋₁₀ heteroaryl, 5- to 10-membered (e.g. C₅₋₁₀) heterocyclyl, 5- to 10-membered (e.g. C₅₋₁₀) carbocyclyl, OR²⁰, N(R²⁰)₂ and halogen; and wherein each aryl, heteroaryl, heterocyclyl and carbocyclyl group is independently unsubstituted or is substituted with 1, 2 or 3, preferably 1 or 2 substituents independently selected from R²⁰, OR²⁰, N(R²⁰)₂ and halogen;
   R^{A} is H or C₁0.4 alkyl which is unsubstituted or is substituted with 1, 2 or 3 substituents independently selected from OR²⁰, N(R²⁰)₂ and halogen;
   R^{B} is H or C₁0.4 alkyl which is unsubstituted or is substituted with 1, 2 or 3 substituents independently selected from OR²⁰, N(R²⁰)₂ and halogen;
   R^{C} is H or C₁0.4 alkyl which is unsubstituted or is substituted with 1, 2 or 3 substituents independently selected from OR²⁰, N(R²⁰)₂ and halogen; and
   each R¹⁰, which may be the same or different, is independently H or C₁0.4 alkyl which is unsubstituted or substituted with 1, 2 or 3 substituents independently selected from OR²⁰, N(R²⁰)₂ and halogen;
   each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl; preferably each R²⁰ is independently H or methyl.

In one embodiment the compound is a compound of Formula (Ib) as described above, each aryl, heteroaryl, heterocyclyl and carbocyclyl group is independently unsubstituted or is substituted with 1 or 2 substituents independently selected from R²⁰, OR²⁰, N(R²⁰)₂ and halogen;
and each R²⁰, which may be the same or different, is independently H or methyl.

In one embodiment the compound is a compound of Formula (B) or Formula (Ib) as described above and R¹ and R² are as defined according to alternative A. In one embodiment the compound is a compound of Formula (B) or Formula (Ib) as described above and R¹ and R² are as defined according to alternative B.

Typically, in Formula (B) or Formula (Ib), X¹ is C—R and X² is C—R. Typically, when X¹ is C—R and X² is C—R, each R is different. In some embodiments, X¹ is CR¹ where R¹ is as defined herein. In some embodiments, X² is CR² where R² is as defined herein.

Typically, in Formula (B) or Formula (Ib), R^{A} is H or C₁₋₃ alkyl, more typically H or C₁₋₂ alkyl, more typically H or C₁ alkyl, most typically H. When R^{A} is an alkyl group, the alkyl group is typically unsubstituted or is substituted with 1 or 2 substituents independently selected from OR²⁰, N(R²⁰)₂ and halogen, more often by OH, NH₂ or halogen. More often, when R^{A} is an alkyl group, the alkyl group is unsubstituted or is substituted with 1 OH or NH₂ group.

Typically, in Formula (B) or Formula (Ib), R^{B} is H or C₁0.3 alkyl, more typically H or C₁₋₂ alkyl, more typically H or C₁ alkyl, most typically H. When R^{B} is an alkyl group, the alkyl group is typically unsubstituted or is substituted with 1 or 2 substituents independently selected from OR²⁰, N(R²⁰)₂ and halogen, more often by OH, NH₂ or halogen. More often, when R^{B} is an alkyl group, the alkyl group is unsubstituted or is substituted with 1 OH or NH₂ group.

Typically, in Formula (B) or Formula (Ib), R^{C} is H or C₁0.3 alkyl, more typically H or C₁₋₂ alkyl, more typically H or C₁ alkyl, most typically H. When R^{C} is an alkyl group, the alkyl group is typically unsubstituted or is substituted with 1 or 2 substituents independently selected from OR²⁰, N(R²⁰)₂ and halogen, more often by OH, NH₂ or halogen. More often, when R^{C} is an alkyl group, the alkyl group is unsubstituted or is substituted with 1 OH or NH₂ group.

Typically, in Formula (B) R^{D} is H or C₁0.3 alkyl, more typically H or C₁₋₂ alkyl, more typically H or C₁ alkyl, most typically H. When R^{D} is an alkyl group, the alkyl group is typically unsubstituted or is substituted with 1 or 2 substituents independently selected from OR²⁰, N(R²⁰)₂ and halogen, more often by OH, NH₂ or halogen. More often, when R^{D} is an alkyl group, the alkyl group is unsubstituted or is substituted with 1 OH or NH₂ group. Thus, typically in Formula (B), X³ is CH₂ or C═O. For instance, X³ may be CH₂. X³ may be C═O.

Typically, the compound is a compound of formula (Ta),

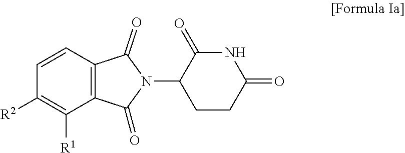

wherein
   A:
      R¹ is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl; and
      R² is selected from N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl;
   or
   B:
      R¹ is selected from OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl; and
      R² is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl;
      wherein each alkyl, alkenyl and alkynyl group is independently unsubstituted or is substituted with 1, 2 or 3 substituents independently selected from C₆₋₁₀ aryl, C₅₋₁₀ heteroaryl, 5- to 10-membered (e.g. C₅₋₁₀) heterocyclyl, 5- to 10-membered (e.g. C₅₋₁₀) carbocyclyl, OR²⁰, N(R²⁰)₂ and halogen;
      and wherein each aryl, heteroaryl, heterocyclyl and carbocyclyl group is independently unsubstituted or is substituted with 1, 2, or 3, preferably 1 or 2 substituents independently selected from R²⁰, OR²⁰, N(R²⁰)₂ and halogen;
   each R¹⁰, which may be the same or different, is independently H or methyl; and
   each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl; preferably each R²⁰ is independently H or methyl.

In one embodiment the compound is a compound of Formula (Ia) as described above, each aryl, heteroaryl, heterocyclyl and carbocyclyl group is independently unsubstituted or is substituted with 1 or 2 substituents independently selected from R²⁰, OR²⁰, N(R²⁰)₂ and halogen; and each R²⁰, which may be the same or different, is independently H or methyl.

Preferably, in Formula (Ia)
A:
   R¹ is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl; and
   R² is selected from NHMe, NMe₂, OMe, chloro, iodo, C₂₋₃ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl;
or
B:
   R¹ is selected from OMe, chloro, bromo, iodo, C₂₋₆ alkyl, C₂₋₆ alkenyl, C₂₋₆ alkynyl, O—C₁0.6 alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl; and
   R² is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered (e.g. C₅₋₁₀) heteroaryl, 5- to 10-membered (e.g. C₅₋₁₀) heterocyclyl, and C₅₋₁₀ carbocyclyl;
   wherein each alkyl, alkenyl and alkynyl group is independently unsubstituted or is substituted with 1, 2 or 3 substituents independently selected from C₆₋₁₀ aryl, C₅₋₁₀ heteroaryl, 5- to 10-membered (e.g. C₅₋₁₀) heterocyclyl, 5- to 10-membered (e.g. C₅₋₁₀) carbocyclyl, OR²⁰, N(R²⁰)₂ and halogen;
   and wherein each aryl, heteroaryl, heterocyclyl and carbocyclyl group is independently unsubstituted or is substituted with 1, 2, or 3, preferably 1 or 2 substituents independently selected from R²⁰, OR²⁰, N(R²⁰)₂ and halogen;
each R¹⁰, which may be the same or different, is independently H or methyl; and
each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl; preferably each R²⁰ is independently H or methyl.

In one embodiment the compound is a compound of Formula (Ia) as described above and R¹ and R² are as defined according to alternative A. In one embodiment the compound is a compound of Formula (Ia) as described above and R¹ and R² are as defined according to alternative B.

In one preferred aspect:
i) R¹ is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl; and
ii) R² is selected from N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl.

In another preferred aspect:
i) R¹ is selected from OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl;
ii) R² is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl.

In another preferred aspect:
i) R¹ is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl; and
ii) R² is selected from NHMe, NMe₂, OMe, chloro, iodo, C₂₋₃ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl.

In another preferred aspect:
i) R¹ is selected from OMe, chloro, bromo, iodo, C₂₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl;
ii) R² is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl.

Typically, when R¹ is or comprises an alkyl or alkynyl group, said alkyl or alkynyl group is independently unsubstituted or is substituted with 1, 2 or 3 substituents independently selected from C₆ aryl, 5- to 6-membered (e.g. C₅₋₆) heteroaryl, and halogen. More typically, said alkyl or alkynyl group is independently unsubstituted or is substituted with 1 or 2 substituents independently selected from C₆ aryl, C₅₋₆ heteroaryl or with 1-3 halogen substituents. Still more typically, said alkyl or alkynyl group is independently unsubstituted or is substituted with 1 or 2 substituents independently selected from C₆ aryl, 5- to 6-membered (e.g. C₅₋₆) heteroaryl or with 1-3 halogen substituents.

When R¹ comprises an aryl, heteroaryl, heterocyclyl or carbocyclyl group, each said group is typically independently unsubstituted or is substituted with 1, 2 or 3 R²⁰ groups, preferably one R²⁰ group. Often said group is unsubstituted. Often said group is substituted by methyl. Sometimes said group is substituted by C₁-C₄ alkyl such as methyl, ethyl, propyl (e.g. ⁱPr), butyl (e.g. ^{t}Bu). Sometimes the group is substituted by 1, 2 or 3 C₁-C₄ alkyl groups such as e.g. 1, 2 or 3 methyl groups.

Typically, when R² is or comprises an alkyl or alkynyl group, said alkyl or alkynyl group is independently unsubstituted or is substituted with 1, 2 or 3 substituents independently selected from C₆ aryl, 5- to 6-membered (e.g. C₅₋₆) heteroaryl, and halogen. More typically, said alkyl or alkynyl group is independently unsubstituted or is substituted with 1 or 2 substituents independently selected from C₆ aryl, 5- to 6-membered (e.g. C₅₋₆) heteroaryl or with 1-3 halogen substituents. Still more typically, said alkyl or alkynyl group is independently unsubstituted or is substituted with 1 or 2 substituents independently selected from C₆ aryl, 5- to 6-membered (e.g. C₅₋₆) heteroaryl or with 1-3 halogen substituents.

When R² comprises an aryl, heteroaryl, heterocyclyl or carbocyclyl group, each said group is typically independently unsubstituted or is substituted with 1, 2 or 3 R²⁰ groups, preferably one R²⁰ group. Often said group is unsubstituted. Often said group is substituted by methyl. Sometimes said group is substituted by C₁-C₄ alkyl such as methyl, ethyl, propyl (e.g. ⁱPr), butyl (e.g. ^{t}Bu). Sometimes the group is substituted by 1, 2 or 3 C₁-C₄ alkyl groups such as e.g. 1, 2 or 3 methyl groups.

In a more preferred aspect:
i) R¹ is selected from H, NH₂, OH, OMe, halogen, methyl, NH—C₁₋₃ alkyl, and unsubstituted NH—C₃ alkynyl; wherein each C₁₋₃ alkyl group is independently unsubstituted or is substituted with 1 or 2 phenyl or pyridinyl groups; wherein said phenyl or pyridinyl groups are each independently unsubstituted or substituted by methyl; and
ii) R² is selected from N(R¹⁰)₂, OH, OMe, halogen, unsubstituted C₁₋₄ alkyl, CF₃, phenyl, piperazinyl, and morpholinyl; wherein said phenyl, piperazinyl, and morpholinyl groups are independently unsubstituted or substituted by methyl.

In another more preferred aspect:
i) R¹ is selected from H, NH₂, OH, OMe, halogen, methyl, NH—C₁₋₃ alkyl, and unsubstituted NH—C₃ alkynyl; wherein each C₁₋₃ alkyl group is independently unsubstituted or is substituted with 1 or 2 phenyl or pyridinyl groups; wherein said phenyl or pyridinyl groups are each independently unsubstituted or substituted by methyl; and
ii) R² is selected from NHMe, NMe₂, OMe, chloro, iodo, unsubstituted C₂₋₃ alkyl, CF₃, phenyl, piperazinyl, and morpholinyl; wherein said phenyl, piperazinyl, and morpholinyl groups are independently unsubstituted or substituted by methyl.

In another more preferred aspect:
i) R¹ is selected from OH, OMe, halogen, methyl, NH—C₁₋₃ alkyl, and unsubstituted NH—C₃ alkynyl; wherein each C₁₋₃ alkyl group is independently unsubstituted or is substituted with 1 or 2 phenyl or pyridinyl groups; wherein said phenyl or pyridinyl groups are each independently unsubstituted or substituted by methyl; and
ii) R² is selected from H, N(R¹⁰)₂, OH, OMe, halogen, unsubstituted C₁₋₄ alkyl, CF₃, phenyl, piperazinyl, and morpholinyl; wherein said phenyl, piperazinyl, and morpholinyl groups are independently unsubstituted or substituted by methyl.

In another more preferred aspect:
i) R¹ is selected from OMe, chloro, bromo, iodo, NH—C₁₋₃ alkyl, and unsubstituted NH—C₃ alkynyl; wherein each C₁₋₃ alkyl group is independently unsubstituted or is substituted with 1 or 2 phenyl or pyridinyl groups; wherein said phenyl or pyridinyl groups are each independently unsubstituted or substituted by methyl; and
ii) R² is selected from H, N(R¹⁰)₂, OH, OMe, halogen, unsubstituted C₁₋₄ alkyl, CF₃, phenyl, piperazinyl, and morpholinyl; wherein said phenyl, piperazinyl, and morpholinyl groups are independently unsubstituted or substituted by methyl.

Often, R¹⁰ is H. Often, R¹⁰ is methyl.

Often, each R²⁰ is independently H or C₁₋₆ alkyl which is independently unsubstituted or is substituted with OH, NH2, or with 1, 2 or 3 halogen substituents. More often, each R²⁰ is independently H or unsubstituted C₁₋₆ alkyl. More often, each R²⁰ is independently H or unsubstituted C₁₋₄ alkyl. More often, each R²⁰ is independently H or unsubstituted C₁₋₃ alkyl. More often, each R²⁰ is independently H or unsubstituted C₁₋₂ alkyl. Often, R²⁰ is H. Often, R²⁰ is methyl.

In another aspect, the compound is a compound of Formula (Ic):

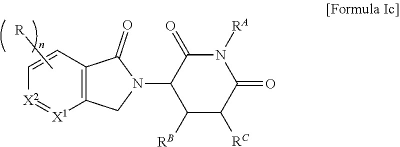

wherein X¹, X², R, n, R^{A}, R^{B}, and R^{C} are as defined above; for example, in some preferred aspects X¹, X², R, n, R^{A}, R^{B}, and R^{C} are as defined for Formula (Ib).

In another aspect, the compound is a compound of Formula (Id):

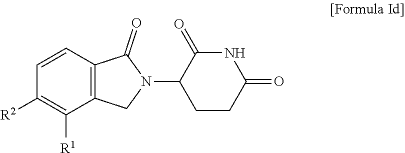

wherein R¹ and R² are as defined above; for example, in some preferred aspects R¹ and R² are as defined for Formula (Ia) and R¹ and R² are according to options A or B above. For example, in a preferred aspect
i) R¹ is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl; and
ii) R² is selected from N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl.

In another preferred aspect:
i) R¹ is selected from OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl;
ii) R² is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered (e.g. C₅₋₆) heterocyclyl.

When R¹ and/or R² comprises an aryl, heteroaryl, heterocyclyl or carbocyclyl group, each said group is typically independently unsubstituted or is substituted with 1, 2 or 3 R²⁰ groups, preferably one R²⁰ group. Often said group is unsubstituted. Often said group is substituted by methyl. Sometimes said group is substituted by C₁-C₄ alkyl such as methyl, ethyl, propyl (e.g. ⁱPr), butyl (e.g. ^{t}Bu). Sometimes the group is substituted by 1, 2 or 3 C₁-C₄ alkyl groups such as e.g. 1, 2 or 3 methyl groups.

In some preferred aspects, the compound is a compound of formula (Ib) or formula (Ic) (preferably formula (Ib)) and X¹ is CR¹ and X² is CR²; or the compound is a compound of formula (Ia) or (Id) (preferably formula (Ia)); and R¹ and R² are according to any of the following. More preferably the compound is a compound of formula (Ib) and X¹ is CR¹ and X² is CR²; or the compound is a compound of formula (Ia); and R¹ and R² are according to any of the following (preferably R¹ and R² are according to any of Compounds 1 to 36 in the following table):

| Compound | | |
|---|---|---|
| number | R¹ | R² |
| 1 | 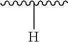 [SMILES: *[H]] | 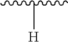 [SMILES: *[H]] |
| 2 | 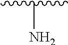 [SMILES: *N] | 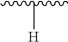 [SMILES: *[H]] |
| 3 | 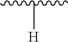 [SMILES: *[H]] | 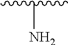 [SMILES: *N] |
| 4 | 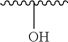 [SMILES: *O] | 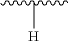 [SMILES: *[H]] |
| 5 | 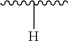 [SMILES: *[H]] | 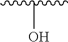 [SMILES: *O] |
| 6 | 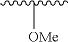 [SMILES: *OC] | 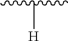 [SMILES: *[H]] |
| 7 | 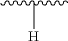 [SMILES: *[H]] | 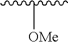 [SMILES: *OC] |
| 8 | 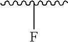 [SMILES: *F] | 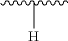 [SMILES: *[H]] |
| 9 | 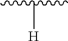 [SMILES: *[H]] | 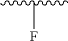 [SMILES: *F] |
| 10 | 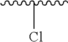 [SMILES: *Cl] | 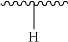 [SMILES: *[H]] |
| 11 | 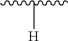 [SMILES: *[H]] | 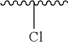 [SMILES: *Cl] |
| 12 | 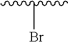 [SMILES: *Br] | 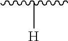 [SMILES: *[H]] |
| 13 | 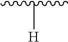 [SMILES: *[H]] | 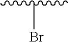 [SMILES: *Br] |
| 14 | 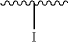 [SMILES: *I] | 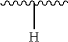 [SMILES: *[H]] |
| 15 | 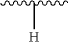 [SMILES: *[H]] | 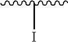 [SMILES: *I] |
| 16 | 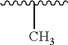 | 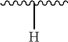 [SMILES: *[H]] |
| 17 | 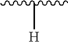 [SMILES: *[H]] | 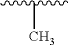 |
| 18 | 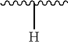 [SMILES: *[H]] | 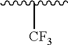 [SMILES: *C(F)(F)F] |
| 19 | 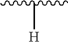 [SMILES: *[H]] | 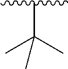 [SMILES: *C(C)(C)C] |
| 20 | 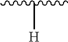 [SMILES: *[H]] | 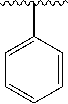 [SMILES: *c1ccccc1] |
| 21 | 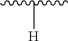 [SMILES: *[H]] | 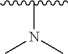 |
| 22 | 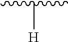 [SMILES: *[H]] | 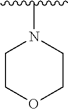 [SMILES: *N1CCOCC1] |
| 23 | 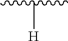 [SMILES: *[H]] | 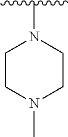 [SMILES: *N1CCN(C)CC1] |
| 24 | 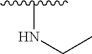 [SMILES: *NCC] | 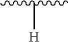 [SMILES: *[H]] |
| 25 | 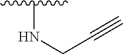 [SMILES: *NCC#C] | 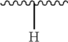 [SMILES: *[H]] |
| 26 | 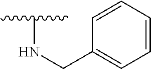 [SMILES: *NCc1ccccc1] | 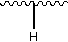 [SMILES: *[H]] |
| 27 | 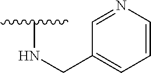 [SMILES: *NCc1cccnc1] | 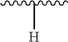 [SMILES: *[H]] |
| 28 | 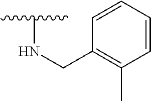 [SMILES: *NCc1ccccc1C] | 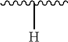 [SMILES: *[H]] |
| 29 | 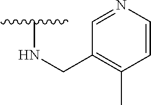 [SMILES: *NCc1cnccc1C] | 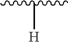 [SMILES: *[H]] |
| 30 | 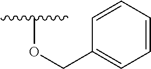 [SMILES: *OCc1ccccc1] | 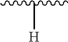 [SMILES: *[H]] |
| 31 | 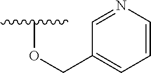 [SMILES: *OCc1cccnc1] | 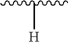 [SMILES: *[H]] |
| 32 | 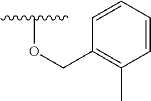 [SMILES: *OCc1ccccc1C] | 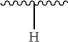 [SMILES: *[H]] |
| 33 | 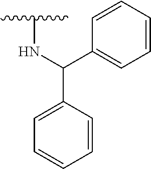 [SMILES: *NC(c1ccccc1)c1ccccc1] | 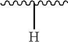 [SMILES: *[H]] |
| 34 | 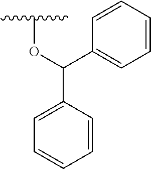 [SMILES: *OC(c1ccccc1)c1ccccc1] | 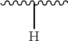 [SMILES: *[H]] |
| 35 | 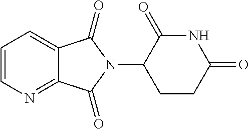 | |
| 36 | 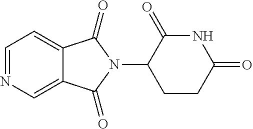 | |
| 37 | 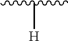 [SMILES: *[H]] | 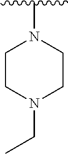 [SMILES: *N1CCN(CC)CC1] |
| 38 | 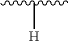 [SMILES: *[H]] | 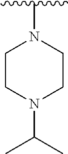 [SMILES: *N1CCN(C(C)C)CC1] |
| 39 | 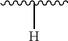 [SMILES: *[H]] | 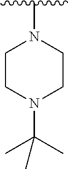 [SMILES: *N1CCN(C(C)(C)C)CC1] |
| 40 | 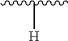 [SMILES: *[H]] | 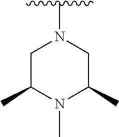 |

The invention provides a kit comprising:
a) a polynucleotide according to the invention, a vector according to the invention, or a cell according to the invention; and
b) a compound according to the system or defined according to the compound of the invention described herein.

The kit provides the components required to implement the system of the invention, wherein a polypeptide and a modified IMiD may function as an inducible degron without affecting other wild type degrons. As a consequence, the kit may allow a user to selectively target a specific protein for degradation. The kit may comprise instructions for use.

### Pharmaceutical Composition

The invention provides a pharmaceutical composition comprising the compound described herein and a pharmaceutically acceptable excipient, carrier or diluent.

Typically, the composition contains up to 85 wt % of a compound of the invention. More typically, it contains up to 50 wt % of a compound of the invention. Preferred pharmaceutical compositions are sterile and pyrogen free.

The carrier may be any suitable carrier known to a person skilled in the art. Carrier proteins include keyhole limpet hemocyanin, serum proteins such as transferrin, bovine serum albumin, human serum albumin, thyroglobulin or ovalbumin, immunoglobulins, or hormones, such as insulin or palmitic acid. Alternatively, the carrier protein may be tetanus toxoid or diphtheria toxoid. Alternatively, the carrier may be a dextran such as sepharose.

Auxiliary substances, such as wetting or emulsifying agents, pH buffering substances and the like, may be present in the excipient. These excipients and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Formulation of a suitable composition can be carried out using standard pharmaceutical formulation chemistries and methodologies all of which are readily available to the reasonably skilled artisan. Such compositions may be prepared, packaged, or sold in a form suitable for bolus administration or for continuous administration. Injectable compositions may be prepared, packaged, or sold in unit dosage form, such as in ampoules or in multi-dose containers optionally containing a preservative. Compositions include, but are not limited to, suspensions, solutions, emulsions in oily or aqueous vehicles, pastes, and implantable sustained-release or biodegradable formulations. In one embodiment of a composition, the active ingredient is provided in dry (for e.g., a powder or granules) form for reconstitution with a suitable vehicle (e. g., sterile pyrogen-free water) prior to administration of the reconstituted composition. The composition may be prepared, packaged, or sold in the form of a sterile injectable aqueous or oily suspension or solution. This suspension or solution may be formulated according to the known art, and may comprise, in addition to the active ingredient, additional ingredients such as the adjuvants, excipients and auxiliary substances described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. Alternatively, the active ingredients of the composition may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

### Method for Degrading a Target Protein and Methods of Therapy

The invention provides a method for degrading a target protein of interest in a cell. As described in more detail herein, the method may be used in an in vitro or ex vivo context. The method may be applied in therapies such as CAR-T cell therapies as described herein.

The method comprises:
a) introducing a polynucleotide or a vector according to the invention into the cell such that the cell expresses the polypeptide attached to the target protein of interest;
b) contacting the cell with a compound according to the system of the invention or as defined according to any compound of the invention described herein;
thereby inducing the degradation of the target protein of interest.

Protein degradation in the method of the invention may be induced following the contacting step (b) by the binding of the compound to the polypeptide encoded by the polynucleotide that is attached to the target protein of interest. This subsequently evokes a stable interaction between the E3 ligase component cereblon (CRBN) and the polypeptide. This interaction brings CRBN and the polypeptide into proximity with one another. Accordingly, CRBN catalyses polyubiquitination of the polypeptide which triggers the proteasomal degradation of the polypeptide and the target protein of interest that is attached to said polypeptide.

In the method of the invention, the polypeptide expressed by the cells may be attached to the protein of interest by genetic fusion and/or wherein the polypeptide is attached to the protein of interest by a linker. The attaching of the polypeptide to the protein target of interest may be defined according to the types of attaching described herein.

The contacting step may involve contacting the cell with any amount of the compound provided that the amount is sufficient to induce degradation of the target protein of interest and provided that, absent any effects of the specific protein degradation, the compound is not cytotoxic to the cell. The contacting preferably comprises the compound entering the cell.

The polynucleotide or vector may be introduced into the cell such that the cell expresses the polypeptide attached to the target protein of interest by any suitable means known in the art.

The polynucleotide or vector may be introduced to the cell by way of transient transfection. The polynucleotide or vector may be stably integrated into the host genome of the cell by any suitable means. The polynucleotide or vector may be integrated into the hose genome of the cell by transposon-based insertion or CRISPR-guided integration.

The cell can be determined to be expressing the polypeptide attached to the target protein of interest by any suitable means, such as for example quantitative PCR or immunodetection.

The cell may be any cell from any organism because the system can be advantageously implemented to investigate the effects of specific protein degradation in any organismal and/or cellular setting.

The cell may preferably be a eukaryotic cell. The cell may be a cell line that is cultured in vitro. The method may be performed entirely in vitro.

The cell may have been obtained from a subject and wherein the introducing step is performed ex vivo. In methods wherein the cell has been obtained from a subject and wherein the introducing step is performed ex vivo, the method may further comprise a step of introducing the cell from step (a) to the same subject, and thus the contacting step (b) then occurs in vivo.

As noted above, the methods may be therapeutic methods. Those skilled in the art will appreciate that the methods disclosed herein have many different therapeutic applications. A number of non-limiting examples are provided herein.

The method may be used in CAR T-cell therapy. Step (a) may comprise the introduction of a polynucleotide or a vector according to the invention into the cell such that the cell expresses the polypeptide attached to the target protein of interest, wherein the cell is a T-cell.

The CAR T-cell therapy may be for the treatment of cancer. The cancer may be any cancer that benefits from CAR T-cell therapy, particularly wherein the cancer is a cancer of the blood. When the CAR T-cell therapy is for the treatment of cancer, the T-cell is preferably a T effector cells (T_{eff}) and wherein the protein target of interest is a Chimeric Antigen Receptor (CAR). Preferably the CAR has an affinity for an antigen expressed by the cancer in the subject. In certain subjects it may desirable to inducibly degrade the CAR and thereby temporarily ‘switch off’ or ‘dampen’ the therapy. For example, the subject may be suffering with debilitating side effects associated with the immune response triggered by the CAR T-cell therapy, such cytokine release syndrome, and thus the subject may for example benefit from a period of dampened CAR T-cell therapy.

The CAR T-cell therapy may be for the treatment of any other disease that would benefit from the therapy, e.g. autoimmune disease, or for the prevention of organ transplant rejection. When the CAR T-cell therapy is for the prevention of organ transplant rejection, the T-cell is preferably a T regulatory cell (T_{reg}) and wherein the protein target of interest is a CAR. Preferably the CAR when expressed by the Treg cell attached to the polypeptide has an immunosuppressive function. The CAR may preferably target antigens expressed by the donor organ and thereby protect said organ from rejection.

The method may be used in gene therapy. Step (a) may comprise the introduction of a polynucleotide or a vector according to the invention into the cell such that the cell expresses the polypeptide attached to the target protein of interest, wherein the cell is a cell type that is desirable for modification by gene therapy. The cell may for example be a stem cell or progenitor cell. The target protein of interest may be a functional and/or genetically modified protein that is for the purpose of compensating for a mutant and/or dysfunctional and/or non-functional endogenous protein in a particular cell type or tissue within the subject. In certain subjects it may desirable to inducibly degrade the compensatory protein in order to ‘dampen’ the effects of gene therapy, for example to reduce any unwanted side effects associated with the gene therapy.

Accordingly, the invention provides a compound as described herein, or a pharmaceutical composition comprising said compound, for use in medicine. Also provided is a compound as described herein, or a pharmaceutical composition comprising said compound for use in treating the human or animal body.

As explained above, the compounds provided herein are useful in treating or preventing various disorders, particularly in subjects who express or otherwise are characterised by one or more of the polypeptides described herein.

Disorders for treatment using the provided compounds, compositions and systems may include cancer, autoimmune disorders (e.g. ankolysing spondylitis), psoriasis, eye disorders such as age-related macular degeneration, multiple sclerosis, cardiovascular disorders, infections including viral and bacterial infections, Crohn's disease, Rheumatoid arthritis, osteoarthritis, Alzheimer's disease, transplant and allograft rejection, etc. hematopoietic stem cell disorders, and the like.

Cancer, e.g. acute lymphoblastic leukemia, acute myeloid leukemia, adrenocortical carcinoma, aids-related lymphoma, primary CNS lymphoma, anal cancer, astrocytomas, brain cancer, basal cell carcinoma, bile duct cancer, bladder cancer, bone cancer (e.g. ewing sarcoma, osteosarcoma and malignant fibrous histiocytoma), breast cancer, bronchial tumors, medulloblastoma and other CNS embryonal tumors, cervical cancer, chronic lymphocytic leukemia, chronic myelogenous leukemia, chronic myeloproliferative neoplasms, colorectal cancer, craniopharyngioma, endometrial cancer, ependymoma, esophageal cancer, esthesioneuroblastoma, ewing sarcoma, extragonadal germ cell tumor, intraocular melanoma, retinoblastoma, fallopian tube cancer, gallbladder cancer, gastric cancer, gastrointestinal carcinoid tumor, gastrointestinal stromal tumors (gist), germ cell tumors, extragonadal germ cell tumors, ovarian germ cell tumors, testicular cancer, gestational trophoblastic disease, hairy cell leukemia, hepatocellular cancer, histiocytosis, langerhans cell, hodgkin lymphoma, hypopharyngeal cancer, intraocular melanoma, islet cell tumors, pancreatic neuroendocrine tumors, kaposi sarcoma, kidney (renal cell) cancer, langerhans cell histiocytosis, laryngeal cancer, leukemia, liver cancer, lung cancer (non-small cell, small cell, pleuropulmonary blastoma, and tracheobronchial tumor), lymphoma, malignant fibrous histiocytoma of bone and osteosarcoma, merkel cell carcinoma, mesothelioma, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, neuroblastoma, non-hodgkin lymphoma, oropharyngeal cancer, osteosarcoma and undifferentiated pleomorphic sarcoma, pancreatic cancer, pancreatic neuroendocrine tumors (islet cell tumors), papillomatosis, paraganglioma, parathyroid cancer, penile cancer, pharyngeal cancer, pheochromocytoma, pituitary tumor, prostate cancer, rectal cancer, retinoblastoma, rhabdomyosarcoma, t-cell lymphoma, testicular cancer, throat cancer, thymoma and thymic carcinoma, thyroid cancer, tracheobronchial tumors and the like is particularly suitable to being treated with the compounds, compositions and systems provided herein.

Autoimmune disorders amenable to being treated with the compounds, compositions and systems provided herein include rheumatoid arthritis, systemic lupus erythematosus (lupus), inflammatory bowel disease (IBD), multiple sclerosis (MS), Type 1 diabetes mellitus, Guillain-Barre syndrome, chronic inflammatory demyelinating polyneuropathy, psoriasis, Graves' disease, Hashimoto's thyroiditis, Myasthenia gravis and vasculitis.

The compounds, compositions and systems provided herein may be used as standalone therapeutic agents. Alternatively, they may be used in combination with other active agents such as chemotherapeutic agents. For example, they may be used in combination with an EGFR inhibitor (for instance erlotinib, gefitinib, lapatinib or cetuximab), an immunotherapy (for instance pembrolizumab or nivolumab), a tumour-agnostic therapy (for instance larotrectinib) or a chemotherapy (for instance 5-fluorouracil, cisplatin or docetaxel).

When used to treat a cancer, the compounds, compositions and systems provided herein may be used in alleviating, ameliorating or preventing aggravation of the symptoms of the cancer. Typically, treating a cancer may comprise reducing progression of the cancer, e.g. increasing progression free survival. Treating a cancer may comprise preventing or inhibiting growth of a tumour associated with the cancer. Treating a cancer may comprise preventing metastasis of the cancer. Preferably, treating a cancer may comprise reducing the size of a tumour associated with the cancer. As such, the treatment may cause tumour regression in the cancer. Treating a cancer may comprise reducing the number of tumours or lesions present in the patient. When the treatment reduces the size of a tumour associated with the cancer, the size of the tumour is typically reduced from base line by at least 10%. Base line is the size of the tumour at the date treatment with the compound is first started. The size of the tumour is typically as measured in accordance with version 1.1 of the RECIST criteria (for instance as described in Eisenhauer et al., European Journal of Cancer 45 (2009) 228-247).

The response to the treatment may be complete response, partial response or stable disease, in accordance with version 1.1 of the RECIST criteria. Preferably, the response is partial response or complete response. The treatment may achieve progression free survival for at least 60 days, at least 120 days or at least 180 days. The reduction in tumour size may be greater 20%, greater than 30% or greater than 50% reduction relative to base line. The reduction in tumour size may be observed after 30 days of treatment or after 60 days of treatment.

As explained here, the compounds, compositions and systems provided herein are useful in treating or preventing various disorders. The present invention therefore provides a compounds, compositions and systems as provided herein for use in medicine. The invention also provides the use of a compounds, compositions and systems as provided herein in the manufacture of a medicament. Also provided is a method of treating a subject in need of such treatment, said method comprising administering to the subject a compound, composition or system provided herein. In some embodiments the subject suffers from or is at risk of suffering from one of the disorders disclosed herein.

In some embodiments the subject expresses or otherwise is characterised by a polypeptide comprising a variant of SEQ ID NO: 1 as described herein. For example, the subject may have been administered CAR-T cell therapy as described herein.

In one aspect, the subject is a mammal, in particular a human. However, it may be non-human. Preferred non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters. The subject can be any animal that is capable of being infected by a bacterium.

A subject is typically a human patient. The patient may be male or female. The age of the patient is typically at least 18 years, for instance from 30 to 70 years or from 40 to 60 years.

An agent or formulation comprising a compound, composition or system of the invention can be administered to the subject in order to prevent the onset or reoccurrence of one or more symptoms of the disorder. This is prophylaxis. In this embodiment, the subject can be asymptomatic. A prophylactically effective amount of the agent or formulation is administered to such a subject. A prophylactically effective amount is an amount which prevents the onset of one or more symptoms of the disorder.

An agent or formulation of the invention can be administered to the subject in order to treat one or more symptoms of the disorder. In this embodiment, the subject is typically symptomatic. A therapeutically effective amount of the agent or formulation is administered to such a subject. A therapeutically effective amount is an amount effective to ameliorate one or more symptoms of the disorder.

The agent may be administered in a variety of dosage forms. Thus, it can be administered orally, for example as tablets, troches, lozenges, aqueous or oily suspensions, dispersible powders or granules. The agent may also be administered parenterally, whether subcutaneously, intravenously, intramuscularly, intrasternally, transdermally or by infusion techniques. The agent may also be administered as a suppository. Preferably, the agent may be administered parenterally.

The agent is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tableting, sugar coating, or film coating processes.

The agent may be formulated for inhaled (aerosolised) administration as a solution or suspension. The agent may be administered by a metered dose inhaler (MDI) or a nebulizer such as an electronic or jet nebulizer. Alternatively, the agent may be formulated for inhaled administration as a powdered drug, such formulations may be administered from a dry powder inhaler (DPI). When formulated for inhaled administration, the agent may be delivered in the form of particles which have a mass median aerodynamic diameter (MMAD) of from 1 to 100 μm, preferably from 1 to 50 μm, more preferably from 1 to 20 μm such as from 3 to 10 μm, e.g. from 4 to 6 μm. When the agent is delivered as a nebulized aerosol, the reference to particle diameters defines the MMAD of the droplets of the aerosol. The MMAD can be measured by any suitable technique such as laser diffraction.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections or inhalation may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for inhalation, injection or infusion may contain as carrier, for example, sterile water or preferably they may be in the form of sterile, aqueous, isotonic saline solutions. Pharmaceutical compositions suitable for delivery by needleless injection, for example, transdermally, may also be used.

A therapeutically or prophylactically effective amount of the agent is administered to a subject. The dose may be determined according to various parameters, especially according to the compound used; the age, weight and condition of the subject to be treated; the route of administration; and the required regimen. Again, a physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific inhibitor, the age, weight and conditions of the subject to be treated, the type and severity of the disease and the frequency and route of administration. Preferably, daily dosage levels are from 5 mg to 2 g.

When the agent is administered to a subject in combination with another active substance (e.g. another medicament as described herein), the dose of the other active substance can be determined as described above. The dose may be determined according to various parameters, especially according to the agent used; the age, weight and condition of the subject to be treated; the route of administration; and the required regimen. Again, a physician will be able to determine the required route of administration and dosage for any particular subject. A typical daily dose is from about 0.01 to 100 mg per kg, preferably from about 0.1 mg/kg to 50 mg/kg, e.g. from about 1 to 10 mg/kg of body weight, according to the activity of the specific agent, the age, weight and conditions of the subject to be treated, the type and severity of the disease and the frequency and route of administration. Preferably, daily dosage levels are from 5 mg to 2 g.

The invention also provides a pharmaceutical composition according to the invention, for use in degrading a therapeutic target protein or a cell comprising a therapeutic target protein in a subject; and preferably wherein said target protein is attached to a polypeptide according to the invention.

In the embodiments of the invention directed to the use of a compound as described herein, or a pharmaceutical composition comprising said compound, for use in medicine, a subject to be treated may have been subject to CAR T-cell therapy, wherein the CAR T-cell therapy comprises:
a) isolation of T-cells from the subject;
b) genetically modifying the isolated T-cells such that isolated T-cells express a chimeric antigen receptor and a polypeptide according to the invention;
c) infusion of the genetically modified T-cells into the subject.

Said use may comprise any of the CAR-T cell therapy steps and features described herein. The chimeric antigen receptor and the polypeptide according to invention maybe attached according to the attaching means described herein, for example by genetic fusion and/or wherein the polypeptide is attached to the chimeric antigen receptor by a linker.

Also provided herein is a polypeptide according to the invention, or a polynucleotide according to the invention, or a vector according to the invention, for use in medicine.

Also provided herein is a polypeptide according to the invention, or a polynucleotide according to the invention, or a vector according to the invention, for use in the treatment or prevention of cancer in a subject in need thereof.

Also provided herein is a polypeptide according to the invention, or a polynucleotide the invention, or a vector according to the invention, for use in a method of CAR T-cell therapy in a subject in need thereof.

### Method for Identifying a System for Degrading a Target Protein

Also provided herein is a method for identifying an orthogonal IMiD compound and polypeptide degron combination, the method comprising screening for increased binding affinity of one or more compounds according to the invention against a polypeptide variant of SEQ ID NO: 2 or SEQ ID NO: 3 relative to a polypeptide sequence consisting of or comprising the polypeptide sequence of SEQ ID NO: 2 or SEQ ID NO: 3, thereby identifying an orthogonal IMiD compound and polypeptide degron combination.

The screening may be any method that allows the user to determine or infer binding affinity of the one or more compounds against the polypeptide variant. For example, the screening may comprise a flow cytometry assay employing cells such as an immortalised human blood cell line, wherein the cells had been stably transduced with a plasmid via a lentiviral vector. This vector may contain a gene for a zinc finger region (ZF) with potential affinity to a compound such as a modified IMiD, such as a ZF region derived from IKZF3, wherein the region is fused to eGFP, separated from mCherry via an internal ribosomal entry site (IRES). Such a screening design would mean that such cells would express ZF-GFP and mCherry in consistent relative amounts, so under normal conditions the ratio of GFP:mCherry would remain the same regardless of overall expression levels. However, upon incubation with an IMID compound, ZF-GFP would be selectively degraded, altering this ratio and thus providing an easily measurable proxy (i.e. an inference) for level of ZF degradation, and thus compound-degron orthogonality. This screening method was used to determine orthogonal ‘hits’ whereby mutant polypeptide degrons were shown to orthogonally interact with specific modified IMiDs with a >2 log FC relative to a DMSO control.

The method for identifying an orthogonal IMiD compound and polypeptide degron combination may be further defined wherein the polypeptide variant is as defined according to the invention and/or wherein the compound is defined according to the invention.

The invention further provides a use of a compound as defined in according to the invention in selectively degrading a target protein of interest in a composition comprising the target protein of interest and a polypeptide of SEQ ID NO: 2 and/or 3.

It is to be understood that although particular embodiments, specific configurations as well as materials and/or molecules, have been discussed herein for methods according to the present disclosure, various changes or modifications in form and detail may be made without departing from the scope and spirit of this invention. The preceding embodiment and following examples are provided for illustration only, and should not be considered limiting the application. The application is limited only by the claims

### EXAMPLES

### Example 1

We aimed to develop a protein-small molecule combination that functions as an inducible degron without affecting other wild type degrons. To achieve this, we employed the bump and hole approach, which involves making a mutant protein with a ‘hole’ that can accommodate a ‘bumped’ ligand that can bind to this mutant, but not the original wild type protein. This approach was pioneered by Schreiber using cyclophilin, and Shokat who applied this approach to generating selective kinase-ligand pairs. More recently, Ciulli has used a bump and hole approach to distinguish between the structurally similar first and second bromodomains of BRD4.

### Materials and Methods—General Chemistry

Reagents and solvents used were of commercially available reagent grade quality from Alfa Aesar, Fluorochem, Merck (formally known as Sigma-Aldrich) and Tokyo Chemical Industry, and were used without further purification, unless stated. Anhydrous solvents were obtained from an MBRAUN Solvent Purification Systems 5 and stored under an argon atmosphere over 3A molecular sieves. Concentration in vacuo was performed at 40° C. for organic solvents using a Buchi™ rotary evaporator. Brine refers to a saturated aqueous solution of sodium chloride. Petroleum ether refers to the fraction of light petroleum ether boiling in the range 40-60° C. Celite® refers to Celite® 545 filter aid, treated with sodium carbonate, flux-calcined (Merck).

¹H NMR spectra were measured on a Bruker AV400 (400 MHz), a Bruker AVII 500 (500 MHz) or a Bruker AV600 (600 MHz) spectrometer in the stated solvents as a reference for the internal deuterium lock. The chemical shift data for each signal are given as δ in units of parts per million (ppm) relative to tetramethylsilane (TMS) where δ(TMS)=0.00. The spectra are calibrated using the solvent peak with the data provided by Fulmer et all. The multiplicity of each signal is indicated by: s (singlet); bs (broad singlet); d (doublet); t (triplet); q (quartet); p (pentet); sept (sept); m (multiplet) or combinations thereof. The number of protons (n) for a given resonance signal is indicated by nH. Where appropriate, coupling constants (J) are quoted in Hz, recorded to the nearest 0.1 Hz, and were determined by analysis using Bruker TopSpin v3.2 software or MestreNova software. Identical coupling constants were averaged. Spectra were assigned using COSY, HSQC and HMBC experiments as necessary. Spectra acquired at high temperature (353 K or 363 K) are indicated.

¹³C NMR spectra were measured on a Bruker AV400 (101 MHz), a Bruker AVII 500 (126 MHz) or a Bruker AV600 (151 MHz) spectrometer in the stated solvents as a reference for the internal deuterium lock using the standard ¹³C experiment. The chemical shift data for each signal are given as δ in units of parts per million (ppm) relative to tetramethylsilane (TMS) where δ(TMS)=0.00. The spectra are calibrated using the solvent peak with the data provided by Fulmer et all. Signals are quoted to 1 decimal place unless peaks are indistinguishable, in which case 2 decimal places are used. Where appropriate, coupling constants (J) are quoted in Hz, recorded to the nearest 0.1 Hz, and were determined by analysis using Bruker TopSpin v3.2 software or MestreNova software. Spectra were assigned using HSQC and HMBC experiments as necessary. Spectra acquired at high temperature (353 K or 363 K) are indicated.

¹⁹F NMR spectra were measured on a Bruker AVIII HD 400 (376 MHz) spectrometer in the solvent stated, and are ¹³C decoupled. Chemical shifts are given as 6 in units of parts per million (ppm), to the nearest 0.1 ppm. The multiplicity of each signal is a singlet unless stated otherwise.

Mass spectra were acquired on either an Agilent 6120 (low resolution) or Bruker microToF spectrometer (high resolution) using electrospray ionisation (ESI) from solutions of either methanol or water. m/z values are reported in Daltons and are followed by their percentage abundance in parentheses.

Melting points were determined using either a) a Leica Galen III hot stage microscope or b) a Griffin capillary tube melting point apparatus (Registered Design No. 889339) and are uncorrected. The solvent(s) from which the sample was crystallised is given in parentheses. Dec. is used to indicate that the sample decomposed at the stated temperature.

Infrared spectra were obtained from thin films, using a diamond ATR module on a Bruker Tensor 27 spectrometer. Absorption maxima are reported in wavenumbers (cm⁻¹) and reported as s (strong), m (medium), w (weak) or br (broad).

Analytical high performance liquid chromatography (HPLC) method 1 was performed on a PerkinElmer Flexar system with a Binary LC Pump and UV/VIS LC Detector set at 254 nm. For determination of purity, a Dionex Acclaim® 120 C18 column [5 μm, 12 Å, 150 mm×4.6 mm]reverse phase column was used with a constant flow rate of 1.5 mL min⁻¹ and gradient method of 10 min from 95:5 H₂O:acetonitrile (0.1% TFA) to 5:95 H₂O:acetonitrile (0.1% TFA) with a 5 min hold. Samples injected were prepared by dissolving in methanol, water or acetonitrile, and filtered. All samples are run with 0.1% TFA added unless indicated.

All biologically assessed compounds had a purity of ≥95%.

Normal Phase silica gel analytical Thin Layer Chromatography (NP TLC) to monitor reaction progress was carried out on normal phase Merck silica gel 60 F254 aluminium-supported thin layer chromatography sheets. Visualisation was carried out by absorption of UV light (λmax=254 nm and 365 nm) or thermal development after staining in an ethanolic solution of ninhydrin or an aqueous solution of potassium permanganate.

Normal phase silica gel flash column chromatography was carried out manually on Merck Geduran® silica gel 60 (40-63 μm), eluting with solvents as supplied, under a positive pressure of compressed, gaseous nitrogen.

### Materials and Methods—Compound Synthesis Procedures and Data

### 2-(2,6-dioxopiperidin-3-yl)isoindoline-1,3-dione

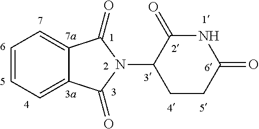 [SMILES: O=C1CCC(N2C(=O)c3ccccc3C2=O)C(=O)N1]

Phthalic anhydride (200 mg, 1.35 mmol, 1.0 eq), 3-aminopiperidine-2,6-dione hydrochloride (222 mg, 1.35 mmol, 1.0 eq) and NaOAc (166 mg, 2.03 mmol, 1.5 eq) were dissolved in AcOH (5 mL) and stirred at 120° C. for 16.5 h. The reaction was cooled to rt and the solvent removed in vacuo (azeotrope with cyclohexane). The product was purified by flash column chromatography (2-10% MeOH/CH₂Cl₂) to afford the title compound (269.8 mg, 77%) as a colourless solid: R_{f} 0.60 (MeOH/CH₂Cl₂ 1:9); m.p. 254-256° C. (from CH₂Cl₂); ¹H NMR (400 MHz, DMSO-d₆) δ 11.12 (s, 1H, H-1′), 7.98-7.85 (m, 4H, H-4, 5, 6, 7), 5.16 (dd, J 12.9, 5.4 Hz, 1H, H-3′), 2.90 (ddd, J 17.5, 14.1, 5.4 Hz, 1H, H-4′, 5′), 2.66-2.52 (m, 2H, H-4′, 5′), 2.13-1.97 (m, 1H, H-4′, 5′); LRMS m/z (ESI⁻) 257 ([M-H]⁻, 100%); HPLC Retention time 220: 6.7 min, 100.0%; 254: 6.6 min, 100.0%.

### 4-amino-2-(2,6-dioxopiperidin-3-yl)isoindoline-1,3-dione

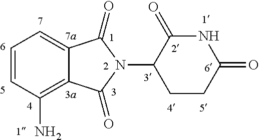 [SMILES: Nc1cccc2c1C(=O)N(C1CCC(=O)NC1=O)C2=O]

2-(2,6-dioxopiperidin-3-yl)-4-nitroisoindoline-1,3-dione (497 mg, 1.64 mmol, 3.0) and palladium on carbon (10%; 50 mg, 0.47 mmol, 1.0 eq) were dissolved in DMF (20 mL) and heated to 35° C. under a H₂ atmosphere for 16 h. The mixture was cooled to rt and filtered through Celite®. The DMF was removed from the filtrate in vacuo (azeotrope with toluene). The residue (light green powder) was dissolved in 20 mL of EtOAc and refluxed at 77° C. for 30 min. The solution was cooled, filtered, and solvent removed in vacuo to afford the title compound (363 mg, 81%) as a yellow, fluorescent powder: R_{f} 0.22 (EtOAc/petroleum ether 1:1); m.p. 290° C. —decomposed (from CH₂Cl₂); ¹H NMR (400 MHz, DMSO-d₆) δ 11.07 (s, 1H, H-1′), 7.47 (dd, J 8.5, 7.0 Hz, 1H, H-7), 7.05-6.97 (m, 2H, H-5, 6), 6.51 (s, 2H, H-1″), 5.04 (dd, J 12.9, 5.4 Hz, 1H, H-3′), 2.88 (ddd, J 17.4, 14.4, 5.4 Hz, 1H, H-4′, 5′), 2.64-2.51 (m, 2H, H-4′, 5′), 2.07-1.96 (m, 1H, H-4′, 5′); LRMS m/z (ESI⁻) 272 ([M-H]⁻, 100%); HPLC Retention time 220: 6.4 min, 99.9%; 254: 6.4 min, 100.0%.

### 5-amino-2-(2,6-dioxopiperidin-3-yl)isoindoline-1,3-dione

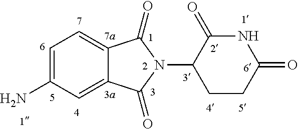 [SMILES: Nc1ccc2c(c1)C(=O)N(C1CCC(=O)NC1=O)C2=O]

2-(2,6-dioxopiperidin-3-yl)-5-nitroisoindoline-1,3-dione (691 mg, 2.28 mmol, 3.0 eq) and palladium on carbon (10%; 70 mg, 0.74 mmol, 1.0 eq) were dissolved in DMF and heated to 35° C. under an argon atmosphere. H₂ (g) was then bubbled through the solution for 10 min and the reaction was stirred at r.t. for 16 h. The mixture was then filtered through Celite® and the residue washed with toluene. The DMF was removed from the filtrate in vacuo (azeotrope with toluene). The residue (dark green powder) was dissolved in 25 mL of EtOAc and refluxed at 77° C. for 30 min. The solution was cooled, filtered, and solvent removed in vacuo to afford a yellow, fluorescent powder (369 mg, 52%): R_{f} 0.40 (MeOH/CH₂Cl₂ 1:9); m.p. >300° C. —decomposed (from CH₂Cl₂); ¹H NMR (400 MHz, DMSO-d₆) δ 11.06 (s, 1H, H-1′), 7.52 (d, J 8.3 Hz, 1H, H-7), 6.94 (d, J 2.0 Hz, 1H, H-4), 6.83 (dd, J 8.2, 2.1 Hz, 1H, H-6), 6.55 (s, 2H, H-1″), 5.01 (dd, J 12.8, 5.5 Hz, 1H, H-3′), 2.94-2.80 (m, 1H, H-4′, 5′), 2.62-2.52 (m, 2H, H-4′, 5′), 2.04-1.93 (m, 1H, H-4′, 5′); LRMS m/z (ESI⁻) 272 ([M-H]⁻, 100%); HPLC Retention time 220: 5.8 min, 100.0%; 254: 5.9 min, 99.9%. 2-(2,6-dioxopiperidin-3-yl)-4-hydroxyisoindoline-1,3-dione

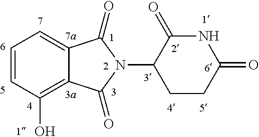 [SMILES: O=C1CCC(N2C(=O)c3cccc(O)c3C2=O)C(=O)N1]

3-hydroxyphthalic anhydride (2 g, 12.19 mmol, 1.0 eq), 3-aminopiperidine-2,6-dione hydrochloride (2.01 g, 12.19 mmol, 1.0 eq) and NaOAc (1.50 g, 18.29 mmol, 1.5 eq) were dissolved in AcOH (50 mL) and heated at 140° C. under reflux for 17.5 h. The AcOH was removed in vacuo (azeotrope with cyclohexane). The residue was dissolved in water and extracted with EtOAc, washed with water and brine, then dried with sodium sulfate. The EtOAc was removed in vacuo to afford a salmon-pink solid (1.14 g, 34%). No further purification was required: R_{f} 0.31 (MeOH/CH₂Cl₂ 1:19); m.p. 257-259° C. (from CH₂Cl₂); ¹H NMR (400 MHz, DMSO-d₆) δ 11.19 (s, 1H, H-1″), 11.08 (s, 1H, H-1′), 7.65 (dd, J 8.4, 7.2 Hz, 1H, H-7), 7.32 (dd, J 7.2, 0.7 Hz, 1H, H-6), 7.25 (dd, J 8.4, 0.8 Hz, 1H, H-5), 5.07 (dd, J 12.8, 5.4 Hz, 1H, H-3′), 2.95-2.81 (m, 1H, H-4′, 5′), 2.64-2.50 (m, 2H, H-4′, 5′), 2.08-1.96 (m, 1H, H-4′, 5′); LRMS m/z (ESI⁻) 273 ([M-H]⁻, 100%); HPLC Retention time 220: 5.9 min, 100.0%; 254: 5.8 min, 100%.

### 2-(2,6-dioxopiperidin-3-yl)-5-hydroxyisoindoline-1,3-dione

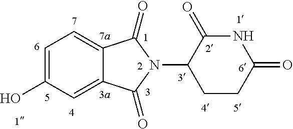 [SMILES: O=C1CCC(N2C(=O)c3ccc(O)cc3C2=O)C(=O)N1]

4-hydroxyphthalic acid (246 mg, 1.35 mmol, 1.0 eq), 3-aminopiperidine-2,6-dione hydrochloride (222 mg, 1.35 mmol, 1.0 eq) and NaOAc (166 mg, 2.03 mmol, 1.5 eq) were dissolved in AcOH (5 mL) and heated at 120° C. under reflux for 3 h. The AcOH was removed in vacuo and the residue purified by flash column chromatography (5% MeOH/CH₂Cl₂) to afford a colourless solid (274 mg, 74%) which fluoresces yellow when dissolved: Rf 0.43 (MeOH/CH₂Cl₂ 1:9); m.p. 200-210° C. (from CH₂Cl₂); ¹H NMR (400 MHz, DMSO-d₆) δ 11.14 (bs, 1H, H-1″), 11.11 (s, 1H, H-1′), 7.74 (dt, J 8.0, 1.0 Hz, 1H, H-7), 7.16 (d, J 7.8 Hz, 2H, H-4, 6), 5.08 (dd, J 12.9, 5.3 Hz, 1H, H-3′), 2.88 (ddd, J 17.1, 13.7, 5.3 Hz, 1H, H-4′, 5′), 2.64-2.51 (m, 2H, H-4′, 5′), 2.11-1.97 (m, 1H, H-4′, 5′). LRMS m/z (ESI⁻) 273 ([M-H]⁻, 100%); HPLC Retention time 220: 6.1 min, 100.0%; 254: 6.0 min, 100.0%.

### 2-(2,6-dioxopiperidin-3-yl)-4-methoxyisoindoline-1,3-dione

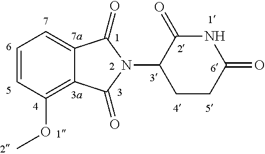 [SMILES: COc1cccc2c1C(=O)N(C1CCC(=O)NC1=O)C2=O]

3-methoxyphthalic acid (125 mg, 0.70 mmol, 1.0 eq), 3-aminopiperidine-2,6-dione hydrochloride (115 mg, 0.70 mmol, 1.0 eq) and NaOAc (86 mg, 1.05 mmol, 1.5 eq) were dissolved in AcOH (5 mL) and heated at 120° C. at reflux for 16.5 h. The AcOH was removed in vacuo and the residue purified by flash column chromatography (2% MeOH/CH₂Cl₂) to afford a colourless solid (96 mg, 47%): R_{f} 0.50 (MeOH/CH₂Cl₂ 1:49); m.p. 271-273° C. (from CH₂Cl₂); ¹H NMR (400 MHz, DMSO-d₆) δ 11.10 (s, 1H, H-1′), 7.84 (dd, J 8.5, 7.3 Hz, 1H, H-6), 7.53 (dd, J 8.6, 0.7 Hz, 1H, H-7), 7.46 (dd, J 7.3, 0.7 Hz, 1H, H-5), 5.09 (dd, J 12.7, 5.5 Hz, 1H, H 3′), 3.97 (s, 3H, H-2″), 2.89 (ddd, J 16.8, 13.7, 5.2 Hz, 1H, H-4′, 5′), 2.63-2.51 (m, 2H, H-4′, 5′), 2.11-1.97 (m, 1H, H-4′, 5′); LRMS m/z (ESI⁻) 287 ([M-H]⁻, 100%); HPLC Retention time 220: 6.5 min, 99.5%; 254: 6.5 min, 100.0%.

### 2-(2,6-dioxopiperidin-3-yl)-5-methoxyisoindoline-1,3-dione

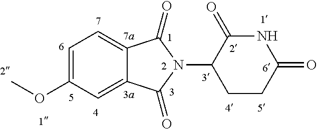 [SMILES: COc1ccc2c(c1)C(=O)N(C1CCC(=O)NC1=O)C2=O]

4-methoxyphthalic acid (167 mg, 0.85 mmol, 1.0 eq), 3-aminopiperidine-2,6-dione hydrochloride (140 mg, 0.85 mmol, 1.0 eq) and NaOAc (105 mg, 1.28 mmol, 1.5 eq) were dissolved in AcOH (5 mL) and heated at 120° C. at reflux for 20 h. The AcOH was removed in vacuo and the residue purified by flash column chromatography (2% MeOH/CH₂Cl₂) to afford a colourless solid (122 mg, 50%): R_{f} 0.61 (MeOH/CH₂Cl₂ 1:49); m.p. 208-212° C. (from CH₂Cl₂); vₘₐₓ (thin film)/cm⁻¹ 1717 (C═O, s); ¹H NMR (400 MHz, DMSO-d₆) δ 11.11 (s, 1H, H-1′), 7.85 (d, J 8.3 Hz, 1H, H-7), 7.44 (d, J 2.3 Hz, 1H, H-4), 7.36 (dd, J 8.3, 2.3 Hz, 1H, H-6), 5.12 (dd, J 12.9, 5.4 Hz, 1H, H-3′), 3.94 (s, 3H, H-2″), 2.89 (ddd, J 17.5, 14.1, 5.6 Hz, 1H, H-4′, 5′), 2.65-2.51 (m, 2H, H-4′, 5′), 2.04 (dtd, J 13.5, 6.3, 3.7 Hz, 1H, H-4′, 5′); “C NMR (151 MHz, DMSO-d₆) δ 172.73 (s, C-6′), 169.89 (s, C-2′), 166.86 (s, C-3), 166.79 (s, C-1), 164.67 (s, C-5), 133.92 (s, C-3a), 125.29 (s, C-7), 123.02 (s, C-7a), 120.26 (s, C-6), 108.53 (s, C-4), 56.38 (s, C-2”), 48.95 (s, C-3′), 30.93 (s, C-5′), 22.04 (s, C-4′); HRMS m/z (EI⁻) [Found: 287.0676, C₁₄H₁₁N₂O₅ requires M-287.0673)]; LRMS m/z (ESI⁻) 287 ([M-H]⁻, 100%); HPLC Retention time 220: 7.2 min, 100.0%; 254: 7.3 min, 100.0%.

### 2-(2,6-dioxopiperidin-3-yl)-4-fluoroisoindoline-1,3-dione

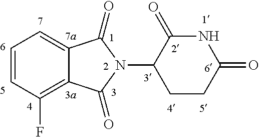 [SMILES: O=C1CCC(N2C(=O)c3cccc(F)c3C2=O)C(=O)N1]

3-fluorophthalic anhydride (2.00 g, 12.04 mmol, 1.0 eq), 3-aminopiperidine-2,6-dione hydrochloride (1.98 g, 12.04 mmol, 1.0 eq) and NaOAc (1.27 g, 18.06 mmol, 1.5 eq) were dissolved in AcOH (50 mL) and heated at 140° C. at reflux for 17 h. The mixture was cooled to rt and the AcOH removed in vacuo (azeotrope with cyclohexane). The residue was extracted from 0.5 M LiCl solution with EtOAc. The EtOAc was removed in vacuo and the residue purified by flash column chromatography (60% EtOAc in petroleum ether) to afford the title compound (3.181 g, 67%) as a colourless solid: R_{f} 0.50 (MeOH/CH₂Cl₂ 1:9); m.p. 231-233° C. (from CH₂Cl₂); ¹H NMR (400 MHz, DMSO-d₆) δ 11.16 (s, 1H, H-1′), 7.95 (td, J 8.2, 5.9 Hz, 1H, H-7), 7.81-7.70 (m, 2H, H-5, 6), 5.16 (dd, J 12.7, 5.4 Hz, 1H, H-3′), 2.89 (ddd, J 17.4, 13.8, 5.5 Hz, 1H, H-4′, 5′), 2.70-2.50 (m, 2H, H-4′, 5′), 2.10-2.02 (m, 1H, H-4′, 5′); 19F NMR (376 MHz, DMSO-d₆) δ −114.69; LRMS m/z (ESI⁻) 275 ([M-H]⁻, 100%); HPLC Retention time 220: 6.8 min, 97.5%; 254: 6.8 min, 98.5%.

### 2-(2,6-dioxopiperidin-3-yl)-5-fluoroisoindoline-1,3-dione

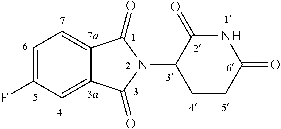 [SMILES: O=C1CCC(N2C(=O)c3ccc(F)cc3C2=O)C(=O)N1]

4-fluorophthalic anhydride (2.00 g, 12.04 mmol, 1.0 eq), 3-aminopiperidine-2,6-dione hydrochloride (1.98 g, 12.04 mmol, 1.0 eq) and NaOAc (1.27 g, 18.06 mmol, 1.5 eq) were dissolved in AcOH (50 mL) and heated at 120° C. at reflux for 1.5 h. The AcOH was removed in vacuo and the residue purified by flash column chromatography (5% MeOH/CH₂Cl₂) to afford a light lilac solid (1.922 g, 58%): R_{f} 0.50 (MeOH/CH₂Cl₂ 1:19); m.p. 240-242° C. (from CH₂Cl₂); ¹H NMR (400 MHz, DMSO-d₆) δ 11.15 (s, 1H, H-1′), 8.01 (dd, J 8.2, 4.5 Hz, 1H, H-4), 7.86 (dd, J 7.5, 2.4 Hz, 1H, H-7), 7.73 (ddd, J 9.4, 8.2, 2.4 Hz, 1H, H-6), 5.17 (dd, J 12.7, 5.4 Hz, 1H, H-3′), 2.89 (ddd, J 17.1, 13.6, 5.4 Hz, 1H, H-4′, 5′), 2.65-2.51 (m, 2H, H-4′, 5′), 2.07 (ddt, J 11.5, 6.4, 4.0 Hz, 1H, H-4′, 5′); 19F NMR (376 MHz, DMSO-d₆) δ −102.35; LRMS m/z (ESI⁻) 275 ([M-H]⁻, 100%); HPLC Retention time 220: 7.1 min, 99.5%; 254: 7.1 min, 100.0%.

### 4-chloro-2-(2,6-dioxopiperidin-3-yl)isoindoline-1,3-dione

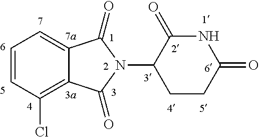 [SMILES: O=C1CCC(N2C(=O)c3cccc(Cl)c3C2=O)C(=O)N1]

3-chlorophthalic anhydride (155 mg, 0.85 mmol, 1.0 eq), 3-aminopiperidine-2,6-dione hydrochloride (140 mg, 0.85 mmol, 1.0 eq) and NaOAc (105 mg, 1.28 mmol, 1.5 eq) were dissolved in AcOH (5 mL) and heated at 120° C. at reflux for 20 h. The AcOH was removed in vacuo and the residue purified by flash column chromatography (2% MeOH/CH₂Cl₂) to afford a colourless solid (19 mg, 8%): R_{f} 0.64 (MeOH/CH₂Cl₂ 1:19); m.p. 272-275° C. (from CH₂Cl₂); ¹H NMR (400 MHz, DMSO-d₆) δ 11.15 (s, 1H, H-1′), 7.95-7.84 (m, 3H, H-5, 6, 7), 5.17 (dd, J 12.8, 5.4 Hz, 1H, H-3′), 2.89 (ddd, J 17.0, 13.9, 5.5 Hz, 1H, H-4′, 5′), 2.66-2.50 (m, 2H, H-4′, 5′), 2.07 (dtd, J 13.0, 5.3, 2.2 Hz, 1H, H-4′, 5′); LRMS m/z (ESI⁻) 291 ([M-H]⁻, 100%), 293 ([M-H]⁻, 33%); HPLC Retention time 220: 7.4 min, 99.2%; 254: 7.4 min, 97.5%.

### 5-chloro-2-(2,6-dioxopiperidin-3-yl)isoindoline-1,3-dione

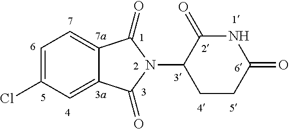 [SMILES: O=C1CCC(N2C(=O)c3ccc(Cl)cc3C2=O)C(=O)N1]

4-chlorophthalic anhydride (155 mg, 0.85 mmol, 1.0 eq), 3-aminopiperidine-2,6-dione hydrochloride (140 mg, 0.85 mmol, 1.0 eq) and NaOAc (105 mg, 1.28 mmol, 1.5 eq) were dissolved in AcOH (5 mL) and heated at 120° C. at reflux for 20 h. The AcOH was removed in vacuo and the residue purified by flash column chromatography (2% MeOH/CH₂Cl₂) to afford a pale pink solid (68 mg, 28%): R_{f} 0.50 (MeOH/CH₂Cl₂ 1:49); m.p. >300° C. (from CH₂Cl₂); ¹H NMR (400 MHz, DMSO-d₆) δ 11.15 (s, 1H, H-1′), 8.04 (t, J 1.2 Hz, 1H, H-7), 7.95 (d, J 1.3 Hz, 2H, H-4, 6), 5.17 (dd, J 12.8, 5.4 Hz, 1H, H-3′), 2.89 (ddd, J 17.0, 13.9, 5.5 Hz, 1H, H-4′, 5′), 2.65-2.52 (m, 2H, H-4′, 5′), 2.06 (dtd, J 12.9, 5.2, 2.2 Hz, 1H, H-4′, 5′); LRMS m/z (ESI⁻) 291 ([M-H]⁻, 100%), 293 ([M-H]⁻, 33%); HPLC Retention time 220: 7.9 min, 95.2%; 254: 7.9 min, 100.0%.

### 4-bromo-2-(2,6-dioxopiperidin-3-yl)isoindoline-1,3-dione

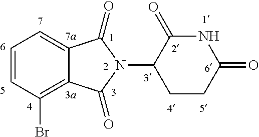 [SMILES: O=C1CCC(N2C(=O)c3cccc(Br)c3C2=O)C(=O)N1]

3-bromophthalic anhydride (200 mg, 0.84 mmol, 1.0 eq), 3-aminopiperidine-2,6-dione hydrochloride (137 mg, 0.84 mmol, 1.0 eq) and NaOAc (103 mg, 1.25 mmol, 1.5 eq) were dissolved in AcOH (5 mL) and heated at 120° C. at reflux for 15 h. The AcOH was removed in vacuo and the residue purified by flash column chromatography (2% MeOH/CH₂Cl₂) to afford a colourless solid (115 mg, 41%): R_{f} 0.68 (MeOH/CH₂Cl₂ 1:19); m.p. 283-286° C. (from CH₂Cl₂); ¹H NMR (400 MHz, DMSO-d₆) δ 11.15 (s, 1H, H-1′), 8.07 (dd, J 8.1, 0.8 Hz, 1H, H-7), 7.94 (dd, J 7.4, 0.9 Hz, 1H, H-5), 7.78 (dd, J 8.1, 7.4 Hz, 1H, H-6), 5.17 (dd, J 12.8, 5.4 Hz, 1H, H-3′), 2.89 (ddd, J 17.0, 13.9, 5.4 Hz, 1H, H-4′, 5′), 2.66-2.50 (m, 2H, H-4′, 5′), 2.12-2.01 (m, 1H, H-4′, 5′); LRMS m/z (ESI⁻) 335 ([M-H]⁻, 97%), 337 ([M-H]⁻, 100%); HPLC Retention time 220: 7.6 min, 100.0%; 254: 7.6 min, 100.0%.

### 5-bromo-2-(2,6-dioxopiperidin-3-yl)isoindoline-1,3-dione

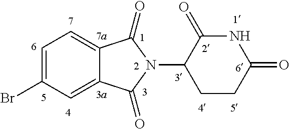 [SMILES: O=C1CCC(N2C(=O)c3ccc(Br)cc3C2=O)C(=O)N1]

4-bromophthalic anhydride (lg, 4.18 mmol, 1.0 eq), 3-aminopiperidine-2,6-dione hydrochloride (688 mg, 4.18 mmol, 1.0 eq) and NaOAc (514 mg, 6.27 mmol, 1.5 eq) were dissolved in AcOH (30 mL) and heated at 120° C. at reflux for 1 h. The AcOH was removed in vacuo and the residue purified by flash column chromatography (5% MeOH/CH₂Cl₂) to afford a colourless solid (585 mg, 42%): R_{f} 0.53 (MeOH/CH₂Cl₂ 1:19); m.p. 292-297—decomposed ° C. (from CH₂Cl₂); ¹H NMR (400 MHz, DMSO-d₆) δ 11.15 (s, 1H, H-1′), 8.15 (dd, J 1.7, 0.6 Hz, 1H, H-4), 8.10 (dd, J 7.9, 1.7 Hz, 1H, H-7), 7.87 (dd, J 7.9, 0.6 Hz, 1H, H-6), 5.17 (dd, J 12.8, 5.4 Hz, 1H, H-3′), 2.89 (ddd, J 17.0, 13.8, 5.4 Hz, 1H, H-4′, 5′), 2.65-2.52 (m, 2H, H-4′, 5′), 2.06 (dtd, J 13.0, 5.3, 2.2 Hz, 1H, H-4′, 5′); LRMS m/z (ESI⁻) 335 ([M-H]⁻, 97%), 337 ([M-H]⁻, 100%); HPLC Retention time 220: 8.2 min, 99.2%; 254: 8.1 min, 97.9%.

### 2-(2,6-dioxopiperidin-3-yl)-4-iodoisoindoline-1,3-dione

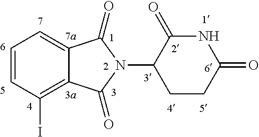 [SMILES: O=C1CCC(N2C(=O)c3cccc(I)c3C2=O)C(=O)N1]

3-iodophthalic acid (300 mg, 1.03 mmol, 1.0 eq), 3-aminopiperidine-2,6-dione hydrochloride (169 mg, 1.03 mmol, 1.0 eq) and NaOAc (126 mg, 1.54 mmol, 1.5 eq) were dissolved in AcOH (5 mL) and heated at 120° C. at reflux for 1 h. The AcOH was removed in vacuo and the residue purified by flash column chromatography (2% MeOH/CH₂Cl₂) to afford a colourless solid (156 mg, 40%): R_{f} 0.60 (MeOH/CH₂Cl₂ 1:19); m.p. 283-285° C. (from CH₂Cl₂); ¹H NMR (400 MHz, DMSO-d₆) δ 11.15 (s, 1H, H-1′), 8.28 (dd, J 7.9, 0.9 Hz, 1H, H-5), 7.93 (dd, J 7.3, 0.9 Hz, 1H, H-7), 7.58 (dd, J 7.9, 7.4 Hz, 1H, H-6), 5.16 (dd, J 12.8, 5.4 Hz, 1H, H-3′), 2.89 (ddd, J 17.0, 13.8, 5.4 Hz, 1H, H-4′, 5′), 2.65-2.52 (m, 2H, H-4′, 5′), 2.06 (dtd, J 12.9, 5.1, 2.1 Hz, 1H, H-4′, 5′); LRMS m/z (ESI⁻) 382.9 ([M-H]⁻, 100%); HPLC Retention time 220: 8.0 min, 100.0%; 254: 7.9 min, 100.0%.

### 2-(2,6-dioxopiperidin-3-yl)-5-iodoisoindoline-1,3-dione

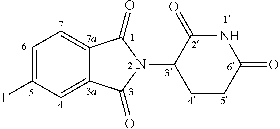 [SMILES: O=C1CCC(N2C(=O)c3ccc(I)cc3C2=O)C(=O)N1]

4-iodophthalic acid (124 mg, 0.43 mmol, 1.0 eq), 3-aminopiperidine-2,6-dione hydrochloride (70 mg, 0.43 mmol, 1.0 eq) and NaOAc (53 mg, 0.64 mmol, 1.5 eq) were dissolved in AcOH (5 mL) and heated at 120° C. at reflux for 2 h. The AcOH was removed in vacuo and the residue purified by flash column chromatography (2% MeOH/CH₂Cl₂) to afford a colourless solid (93 mg, 57%): R_{f} 0.48 (MeOH/CH₂Cl₂ 1:49); m.p. 231-233° C. (from CH₂Cl₂); vₘₐₓ (thin film)/cm⁻¹ 1726 (C═O, s); ¹H NMR (400 MHz, DMSO-d₆) δ 11.14 (s, 1H, H-1′), 8.31-8.24 (m, 2H, H-4, 6), 7.69 (d, J 8.1 Hz, 1H, H-7), 5.15 (dd, J 12.9, 5.4 Hz, 1H, H-3′), 2.88 (ddd, J 17.0, 13.9, 5.4 Hz, 1H, H-4′, 5′), 2.65-2.51 (m, 2H, H-4′, 5′), 2.05 (dtd, J 12.9, 5.2, 2.1 Hz, 1H, H-4′, 5′); ¹³C NMR (151 MHz, DMSO-d₆) δ 172.69 (s, C-6′), 169.67 (s, C-2′), 166.76 (s, C-3), 165.92 (s, C-1), 143.47 (s, C-6), 132.65 (s, C-3a), 131.83 (s, C-4), 130.43 (s, C-7a), 124.92 (s, C-7), 102.76 (s, C-5), 49.07 (s, C-3′), 30.88 (s, C-5′), 21.89 (s, C-4′); HRMS m/z (EI⁻) [Found: 382.9538, C₁₃H₈IN₂O₄ requires M⁻ 382.9534)]; LRMS m/z (ESI⁻) 382.9 ([M-H]⁻, 100%); HPLC Retention time 220: 8.5 min, 100%; 254: 8.6 min, 99.5%.

### 2-(2,6-dioxopiperidin-3-yl)-4-methylisoindoline-1,3-dione

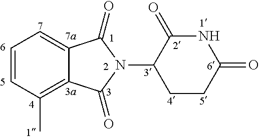 [SMILES: Cc1cccc2c1C(=O)N(C1CCC(=O)NC1=O)C2=O]

3-methylphthalic anhydride (200 mg, 1.204 mmol, 1.0 eq), 3-aminopiperidine-2,6-dione hydrochloride (203 mg, 1.204 mmol, 1.0 eq) and NaOAc (152 mg, 1.806 mmol, 1.5 eq) were dissolved in AcOH (5 mL) and heated at 140° C. under reflux for 24 h. The residue was extracted from 0.5M LiCl solution with EtOAc. The EtOAc was removed in vacuo and the residue purified by flash column chromatography (2% MeOH/CH₂Cl₂) to afford an off-white solid (135 mg, 40%): R_{f} 0.64 (MeOH/CH₂Cl₂ 1:9); m.p. 273-278° C. (from CH₂Cl₂); ¹H NMR (400 MHz, DMSO-d₆) δ 11.11 (s, 1H, H-1′), 7.72-7.64 (m, 3H, H-5, 6, 7), 5.13 (dd, J 12.9, 5.4 Hz, 1H, H-3′), 2.89 (ddd, J 17.4, 14.1, 5.4 Hz, 1H, H-4′, 5′), 2.63 (s, 3H, H-1″), 2.61-2.50 (m, 2H, H-4′, 5′), 2.11-1.99 (m, 1H, H-4′, 5′); LRMS m/z (ESI⁻) 271 ([M-H]⁻, 100%); HPLC Retention time 220: 7.5 min, 100.0%; 254: 7.5 min, 100%.

### 2-(2,6-dioxopiperidin-3-yl)-5-methylisoindoline-1,3-dione

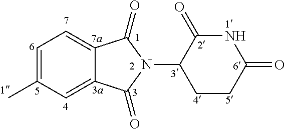 [SMILES: Cc1ccc2c(c1)C(=O)N(C1CCC(=O)NC1=O)C2=O]

4-methylphthalic anhydride (200 mg, 1.204 mmol, 1.0 eq), 3-aminopiperidine-2,6-dione hydrochloride (203 mg, 1.204 mmol, 1.0 eq) and NaOAc (152 mg, 1.81 mmol, 1.5 eq) were dissolved in AcOH (5 mL) and heated at 120° C. under reflux for 2 h. The AcOH was removed in vacuo and the residue purified by flash column chromatography (5% MeOH/CH₂Cl₂) to afford a colourless solid (277 mg, 84.3%): R_{f} 0.60 (MeOH/CH₂Cl₂ 1:19); m.p. 255-260° C. (from CH₂Cl₂); ¹H NMR (400 MHz, DMSO-d₆) δ 11.12 (s, 1H, H-1′), 7.84-7.78 (m, 1H, H-4), 7.76 (dt, J 1.4, 0.7 Hz, 1H, H-7), 7.69 (ddd, J 7.6, 1.5, 0.8 Hz, 1H, H-6), 5.18-5.09 (m, 1H, H-3′), 2.96-2.82 (m, 1H, H-4′, 5′), 2.65-2.52 (m, 2H, H-4′, 5′), 2.11-2.00 (m, 1H, H-4′, 5′); LRMS m/z (ESI⁻) 271 ([M-H]⁻, 100%); HPLC Retention time 220: 7.5 min, 100.0%; 254: 7.5 min, 100%.

### 2-(2,6-dioxopiperidin-3-yl)-5-(trifluoromethyl)isoindoline-1,3-dione

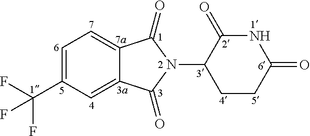 [SMILES: O=C1CCC(N2C(=O)c3ccc(C(F)(F)F)cc3C2=O)C(=O)N1]

4-(trifluoromethyl)phthalic acid (200 mg, 0.850 mmol, 1.0 eq), 3-aminopiperidine-2,6-dione hydrochloride (140 mg, 0.85 mmol, 1.0 eq) and NaOAc (105 mg, 1.28 mmol, 1.5 eq) were dissolved in AcOH (5 mL) and heated at 120° C. under reflux for 20 h. The AcOH was removed in vacuo and the residue purified by flash column chromatography (5% MeOH/CH₂Cl₂) to afford a pale pink solid (192 mg, 69%): Rf 0.75 (MeOH/CH₂Cl₂ 1:9); m.p. 187-193° C. (from CH₂Cl₂); vₘₐₓ (thin film)/cm⁻¹ 1730 (C═O, s); ¹H NMR (400 MHz, DMSO-d₆) δ 11.18 (s, 1H, H-1′), 8.29 (d, J 8.1 Hz, 2H, H-4, 6), 8.16 (d, J 7.7 Hz, 1H, H-7), 5.22 (dd, J 12.8, 5.4 Hz, 1H, H-3′), 2.90 (ddd, J 17.0, 13.8, 5.4 Hz, 1H, H-4′, 5′), 2.67-2.52 (m, 2H, H-4′, 5′), 2.08 (dtd, J 13.0, 5.3, 2.1 Hz, 1H, H-4′, 5′); ¹³C NMR (151 MHz, CDCl₃) δ 172.68 (s, C-6′), 169.56 (s, C-2′), 165.98 (s, C-1), 165.80 (s, C-3), 134.65 (s, C-3a), 134.45 (q, J_{C-F}=32.804 Hz, C-5), 132.23 (s, C-7a), 132.02 (d, J_{C-F}=21.204 Hz, C-6), 124.54 (d, J_{C-F}=22.369 Hz, C-7), 123.18 (q, J_{C-F}=273.104 Hz, C-1″), 120.43 (d, J_{C-F}=21.203 Hz, C-4), 49.29 (s, C-3′), 30.87 (s, C-5′), 21.84 (s, C-4′); 19F NMR (565 MHz, CDCl₃) δ −56.61; HRMS m/z (EI⁻) [Found: 325.0439, C₁₄H₈F₃N₂O₄ requires M⁻ 325.0442)]; LRMS m/z (ESI⁻) 325 ([M-H]⁻, 100%); HPLC Retention time 220: 8.5 min, 100.0%; 254: 9.5 in, 100.0%.

### 5-(tert-butyl)-2-(2,6-dioxopiperidin-3-yl)isoindoline-1,3-dione

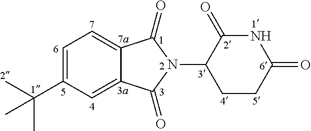 [SMILES: CC(C)(C)c1ccc2c(c1)C(=O)N(C1CCC(=O)NC1=O)C2=O]

4-tert-butylphthalic anhydride (200 mg, 0.98 mmol, 1.0 eq), 3-aminopiperidine-2,6-dione hydrochloride (161 mg, 0.98 mmol, 1.0 eq) and NaOAc (121 mg, 1.47 mmol, 1.5 eq) were dissolved in AcOH (5 mL) and heated at 120° C. under reflux for 21 h. The AcOH was removed in vacuo and the residue purified by flash column chromatography (10% MeOH/CH₂Cl₂) to afford a colourless solid (139 mg, 45%): R_{f} 0.69 (MeOH/CH₂Cl₂ 1:9); m.p. 145-155° C. (from CHCl₃); ¹H NMR (400 MHz, DMSO-d₆) δ 11.12 (s, 1H, H-1′), 7.96-7.82 (m, 3H, H-4, 6, 7), 5.14 (dd, J 12.9, 5.4 Hz, 1H, H-3′), 2.96-2.82 (m, 1H, H-4′, 5′), 2.65-2.52 (m, 2H, H-4′, 5′), 2.11-1.99 (m, 1H, H-4′, 5′), 1.36 (s, 9H, H-1″); LRMS m/z (ESI⁻) 313 ([M-H]⁻, 100%); HPLC Retention time 220: 9.7 min, 99.8%; 254: 9.7 min, 100.0%.

### 2-(2,6-dioxopiperidin-3-yl)-5-phenylisoindoline-1,3-dione

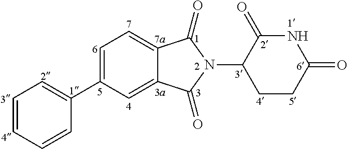 [SMILES: O=C1CCC(N2C(=O)c3ccc(-c4ccccc4)cc3C2=O)C(=O)N1]

5-phenylisobenzofuran-1,3-dione (125 mg, 0.56 mmol, 1.0 eq), 3-aminopiperidine-2,6-dione hydrochloride (92 mg, 0.43 mmol, 1.0 eq) and NaOAc (69 mg, 0.84 mmol, 1.5 eq) were dissolved in AcOH (5 mL) and heated at 120° C. at reflux for 2 h. The AcOH was removed in vacuo and the residue purified by flash column chromatography (2% MeOH/CH₂Cl₂) to afford a colourless solid (119 mg, 64%): R_{f} 0.65 (MeOH/CH₂Cl₂ 1:19); R_{f} 0.65 (MeOH/CH₂Cl₂ 1:19); m.p. 210-220° C. (from CH₂Cl₂); ¹H NMR (400 MHz, DMSO-d₆) δ 11.15 (s, 1H, H-1′), 8.23-8.15 (m, 2H, H-4, 6, 7, 1″, 2″, 3″, 4″), 8.05-7.96 (m, 1H, H-4, 6, 7, 1″, 2″, 3″, 4″), 7.88-7.81 (m, 2H, H-4, 6, 7, 1″, 2″, 3″, 4″), 7.59-7.44 (m, 3H, H-4, 6, 7, 1″, 2″, 3″, 4″), 5.19 (dd, J 12.9, 5.4 Hz, 1H, H-3′), 2.91 (ddd, J 17.3, 14.0, 5.5 Hz, 1H, H-4′, 5′), 2.66-2.52 (m, 2H, H-4′, 5′), 2.14-2.03 (m, 1H, H-4′, 5′); LRMS m/z (ESI⁻) 333 ([M-H]⁻, 100%); HPLC Retention time 220: 9.5 min, 98.8%; 254: 9.5 min, 100.0%.

### 5-(dimethylamino)-2-(2,6-dioxopiperidin-3-yl)isoindoline-1,3-dione

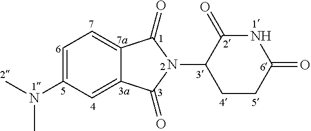 [SMILES: CN(C)c1ccc2c(c1)C(=O)N(C1CCC(=O)NC1=O)C2=O]

2-(2,6-dioxopiperidin-3-yl)-5-fluoroisoindoline-1,3-dione (50 mg, 0.181 mmol, 1.0 eq), dimethylamine (2.0M in THF, 2.90 mmol, 16.0 eq) and DIPEA (504 μL, 2.90 mmol, 16.0 eq) were dissolved in DMF (4 mL) and heated at 110° C. for 21 h. The DMF was removed by vigorously blowing nitrogen over the reaction vessel. The residual dark brown oil was purified by flash chromatography (45-50% EtOAc/petroleum ether) to afford a bright yellow powder (48.1 mg, 88%): R_{f} 0.07 (EtOAc/petroleum ether 9:11); m.p. 241-243° C. (from EtOAc); ¹H NMR (400 MHz, CDCl₃) δ 7.99 (s, 1H, H-1′), 7.67 (d, J 8.5 Hz, 1H, H-7), 7.09 (d, J 2.4 Hz, 1H, H-4), 6.82 (dd, J 8.6, 2.4 Hz, 1H, H-6), 4.98-4.89 (m, 1H, H-3′), 3.13 (s, 6H, H-2″), 2.94-2.64 (m, 3H, H-4′, 5′), 2.21-2.08 (m, 1H, H-4′, 5′); LRMS m/z (ESI⁻) 300 ([M-H]⁻, 100%); HPLC Retention time 220: 7.5 min, 98.9%; 254: 7.5 min, 98.6%.

### (2,6-dioxopiperidin-3-yl)-5-morpholinoisoindoline-1,3-dione

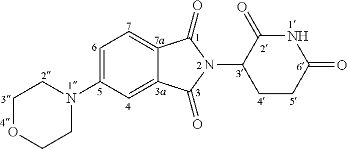 [SMILES: O=C1CCC(N2C(=O)c3ccc(N4CCOCC4)cc3C2=O)C(=O)N1]

2-(2,6-dioxopiperidin-3-yl)-5-fluoroisoindoline-1,3-dione (50 mg, 0.18 mmol, 1.0 eq), morpholine (32 μL, 0.36 mmol, 2.0 eq) and DIPEA (126 μL, 0.72 mmol, 4.0 eq) were dissolved in NMP (2 mL) and heated at 110° C. for 2 h (microwave). The NMP was removed by vigorously blowing nitrogen over the reaction vessel whilst heating to 60° C.. The residual dark brown oil was purified by flash chromatography (40% EtOAc/petroleum ether) to afford a bright yellow solid (23 mg, 37%): R_{f} 0.09 (EtOAc/petroleum ether 1:1); m.p. 198-202° C. (from EtOAc); vₘₐₓ (thin film)/cm⁻¹ 1707 (C═O, s); ¹H NMR (400 MHz, CDCl₃) δ 7.95 (s, 1H, H-1′), 7.73 (d, J 8.5 Hz, 1H, H-7), 7.30 (d, J 2.4 Hz, 1H, H-4), 7.08 (dd, J 8.5, 2.4 Hz, 1H, H-6), 4.95 (dd, J 12.3, 5.3 Hz, 1H, H-3′), 3.88 (dd, J 6.0, 3.8 Hz, 4H, H-3″), 3.41-3.34 (m, 4H, H-2″), 2.95-2.68 (m, 3H, H-4′, 5′), 2.19-2.09 (m, 1H, H-4′, 5′); ¹³C NMR (151 MHz, CDCl₃) δ 170.93 (s, C-6′), 168.23 (s, C-2′), 167.91 (s, C-1), 167.29 (s, C-3), 155.84 (s, C-5), 134.41 (s, C-3a), 125.54 (s, C-7), 120.48 (s, C-7a), 118.11 (s, C-6), 108.75 (s, C-4), 66.50 (s, C-3″), 49.35 (s, C-3′), 47.81 (s, C-2″), 31.59 (s, C-5′), 22.89 (s, C-4′); HRMS m/z (EI⁻) [Found: 342.1098, C₁₇H₁₆N₃O₅ requires M-342.1095)]; LRMS m/z (ESI⁻) 342 ([M-H]⁻, 100%); HPLC Retention time 220: 7.0 min, 96.0%; 254: 7.0 min, 100.0%.

### 2-(2,6-dioxopiperidin-3-yl)-5-(4-methylpiperazin-1-yl)isoindoline-1,3-dione

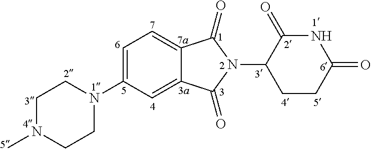 [SMILES: CN1CCN(c2ccc3c(c2)C(=O)N(C2CCC(=O)NC2=O)C3=O)CC1]

2-(2,6-dioxopiperidin-3-yl)-5-fluoroisoindoline-1,3-dione (50 mg, 0.18 mmol, 1.0 eq), 1-methylpiperazine (40 μL, 0.36 mmol, 2.0 eq) and DIPEA (126 μL, 0.72 mmol, 4.0 eq) were dissolved in NMP (2 mL) and heated at 110° C. for 2 h (microwave). The NMP was removed by vigorously blowing nitrogen over the reaction vessel whilst heating to 60° C.. The residual dark brown oil was purified by flash chromatography (2-10% MeOH/CH₂Cl₂) to afford a bright yellow solid (40 mg, 62%): R_{f} 0.44 (MeOH/CH₂Cl₂ 1:9); m.p. 174-178° C. (from EtOAc); vₘₐₓ (thin film)/cm⁻¹ 1709 (C═O, s); ¹H NMR (400 MHz, CDCl₃) δ 8.40 (s, 1H, H-1′), 7.69 (d, J 8.5 Hz, 1H, H-7), 7.28 (d, J 2.3 Hz, 1H, H-4), 7.06 (dd, J 8.6, 2.4 Hz, 1H, H-6), 4.98-4.88 (m, 1H, H-3′), 3.47-3.40 (m, 4H, H-2″), 2.93-2.64 (m, 3H, H-4′, 5′), 2.62-2.53 (m, 4H, H-3″), 2.19-2.02 (m, 1H, H-4′, 5′); ¹³C NMR (151 MHz, CDCl₃) δ 170.99 (s, C-6′), 168.28 (s, C-2′), 168.01 (s, C-3), 167.34 (s, C-1), 155.60 (s, C-5), 134.44 (s, C-3a), 125.54 (s, C-7), 119.66 (s, C-7a), 118.19 (s, C-6), 108.91 (s, C-4), 54.58 (s, C-3″), 49.32 (s, C-3′), 47.56 (s, C-2″), 46.11 (s, C-5″), 31.59 (s, C-5′), 22.90 (s, C-4′); HRMS m/z (EI⁻) [Found: 355.1417, C₁₈H₁₉N₄O₄ requires M-355.1412)]; LRMS m/z (ESI⁻) 355 ([M-H]⁻, 100%); HPLC Retention time 220: 5.2 min, 100.0%; 254: 5.1 min, 100.0%.

### 2-(2,6-dioxopiperidin-3-yl)-4-(ethylamino)isoindoline-1,3-dione

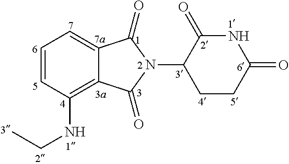 [SMILES: CCNc1cccc2c1C(=O)N(C1CCC(=O)NC1=O)C2=O]

2-(2,6-dioxopiperidin-3-yl)-4-fluoroisoindoline-1,3-dione (200 mg, 0.72 mmol, 1.0 eq), ethylamine (2.0 M in THF; 1.44 mL, 2.90 mmol, 4.0 eq) and DIPEA (504 μL, 2.90 mmol, 4.0 eq) were dissolved in NMP (4 mL) and heated at 110° C. for 2 h (microwave). The NMP was removed by vigorously blowing nitrogen over the reaction vessel whilst heating to 60° C.. The residual oil was purified by flash column chromatography (40% EtOAc in petroleum ether) to afford a yellow powder (6 mg, 3%): R_{f} 0.30 (EtOAc/petroleum ether 3:7); m.p. 198-202° C. (from EtOAc); ¹H NMR (400 MHz, CDCl₃) δ 7.96 (s, 1H, H-1′), 7.50 (ddd, J 8.5, 7.1, 0.6 Hz, 1H, H-6), 7.10 (dd, J 7.1, 0.6 Hz, 1H, H-7), 6.89 (d, J 8.5 Hz, 1H, H-5), 6.17 (s, 1H, H-1″), 4.96-4.87 (m, 1H, H-3′), 3.32 (qd, J 7.2, 5.4 Hz, 2H, H-2″), 2.98-2.64 (m, 3H, H-4′, 5′), 2.19-2.08 (m, 1H, H-4′, 5′), 1.31 (t, J 7.2 Hz, 3H, H-3″); LRMS m/z (ESI⁻) 300 ([M-H]⁻, 100%); HPLC Retention time 220: 8.5 min, 97.9%; 254: 8.5 min, 98.6%.

### 2-(2,6-dioxopiperidin-3-yl)-4-(prop-2-yn-1-ylamino)isoindoline-1,3-dione

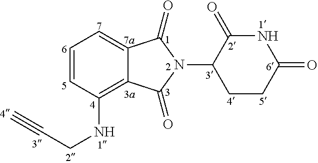 [SMILES: C#CCNc1cccc2c1C(=O)N(C1CCC(=O)NC1=O)C2=O]

2-(2,6-dioxopiperidin-3-yl)-4-fluoroisoindoline-1,3-dione (200 mg, 0.72 mmol, 1.0 eq), propargylamine (186 μL, 2.90 mmol, 4.0 eq) and DIPEA (504.4 μL, 2.896 mmol, 4.0 eq) were dissolved in NMP (4 mL) and heated at 110° C. for 2 h (microwave). The NMP was removed by vigorously blowing nitrogen over the reaction vessel whilst heating to 60° C.. The residual oil was purified by flash column chromatography (30-50% EtOAc/petroleum ether) to afford a bright yellow solid (123 mg, 54%): R_{f} 0.55 (EtOAc/petroleum ether 3:2); m.p. 169-172° C. (from EtOAc); ¹H NMR (400 MHz, CDCl₃) δ 8.03 (s, 1H, H-1′), 7.57 (dd, J 8.5, 7.2 Hz, 1H, H-6), 7.20 (d, J 7.2 Hz, 1H, H-7), 7.03 (d, J 8.5 Hz, 1H, H-5), 6.45 (t, J 6.2 Hz, 1H, H-1″), 4.92 (dd, J 12.2, 5.3 Hz, 1H, H-3′), 4.09 (dd, J 6.1, 2.5 Hz, 2H, H-2″), 2.94-2.67 (m, 3H, H-4′, 5′), 2.27 (t, J 2.5 Hz, 1H, H-4″), 2.18-2.08 (m, 1H, H-4′, 5′); LRMS m/z (ESI⁻) 310 ([M-H]⁻, 100%); HPLC Retention time 220: 8.0 min, 96.7%; 254: 8.1 min, 97.7%.

### 4-(benzylamino)-2-(2,6-dioxopiperidin-3-yl)isoindoline-1,3-dione

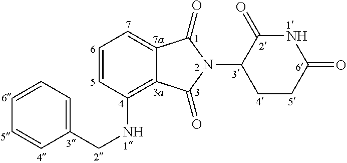 [SMILES: O=C1CCC(N2C(=O)c3cccc(NCc4ccccc4)c3C2=O)C(=O)N1]

2-(2,6-dioxopiperidin-3-yl)-4-fluoroisoindoline-1,3-dione (200 mg, 0.72 mmol, 1.0 eq), benzylamine (158 μL, 1.45 mmol, 2.0 eq) and DIPEA (504 μL, 2.90 mmol, 4.0 eq) were dissolved in NMP (4 mL) and heated at 110° C. for 2 h (microwave). The NMP was removed by blowing nitrogen over it overnight whilst heating the vessel to 60 C. The resultant oil was purified by column chromatography (50% EtOAc/petroleum ether) to afford an ochre/yellow solid (125 mg, 48%): R_{f} 0.40 (EtOAc/petroleum ether 1:1); m.p. 88-98° C. (from EtOAc); ¹H NMR (400 MHz, CDCl₃) δ 8.00 (s, 1H, H-1′), 7.45 (dd, J 8.5, 7.1 Hz, 1H, H-6), 7.40-7.27 (m, 5H, H-4″, 5″, 6″), 7.12 (d, J 7.1 Hz, 1H, H-7), 6.84 (d, J 8.5 Hz, 1H, H-5), 6.70 (t, J 6.0 Hz, 1H, H-1″), 4.97-4.88 (m, 1H, H-3′), 4.52 (d, J 5.9 Hz, 2H, H-2″), 2.94-2.68 (m, 3H, H-4′, 5′), 2.20-2.10 (m, 1H, H-4′, 5′); LRMS m/z (ESI⁻) 362 ([M-H]⁻, 100%); HPLC Retention time 220: 9.8 min, 99.5%; 254: 9.8 min, 97.7%.

### 2-(2,6-dioxopiperidin-3-yl)-4-((pyridin-3-ylmethyl)amino)isoindoline-1,3-dione

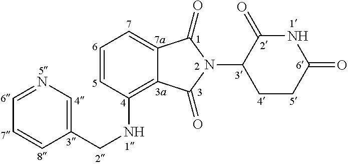 [SMILES: O=C1CCC(N2C(=O)c3cccc(NCc4cccnc4)c3C2=O)C(=O)N1]

2-(2,6-dioxopiperidin-3-yl)-4-fluoroisoindoline-1,3-dione (200 mg, 0.72 mmol, 1.0 eq), 3-picolylamine (147.4 μL, 1.45 mmol, 2.0 eq) and DIPEA (504 μL, 2.90 mmol, 4.0 eq) were dissolved in NMP (4 mL) and heated at 110° C. for 2 h (microwave). The NMP was removed by vigorously blowing nitrogen over the reaction vessel whilst heating to 60° C.. The residual orange oil was purified by flash column chromatography (100% EtOAc/petroleum ether) to afford a bright, light yellow solid (149.2 mg, 57%): R_{f} 0.31 (EtOAc); m.p. 201-204° C. (from EtOAc); vₘₐₓ (thin film)/cm⁻¹ 1696 (C═O, s); ¹H NMR (400 MHz, CDCl₃) δ 8.63 (d, J 2.3 Hz, 1H, H-4″), 8.56 (dd, J 4.8, 1.6 Hz, 1H, H-6″), 8.05 (s, 1H, H-1′), 7.68 (dt, J 7.8, 2.0 Hz, 1H, H-8″), 7.47 (dd, J 8.5, 7.2 Hz, 1H, H-6), 7.30 (dd, J 7.9, 4.8 Hz, 1H, H-7″), 7.16 (d, J 7.1 Hz, 1H, H-7), 6.82 (d, J 8.5 Hz, 1H, H-5), 6.71 (t, J 5.9 Hz, 1H, H-1″), 4.93 (dd, J 12.2, 5.3 Hz, 1H, H-3′), 4.55 (d, J 6.0 Hz, 2H, H-2″), 2.95-2.68 (m, 3H, H-4′, 5′), 2.20-2.12 (m, 1H, H-4′, 5′); ¹³C NMR (151 MHz, CDCl₃) δ 170.91 (s, C-6′), 169.58 (s, C-2′), 168.28 (s, C-1), 167.54 (s, C-3), 149.39 (s, C-4″), 148.95 (s, C-6″), 146.40 (s, C-4), 136.47 (s, C-6), 134.81 (s, C-8″), 133.40 (s, C-3″), 132.68 (s, C-7a), 123.93 (s, C-7″), 117.02 (s, C-5), 112.66 (s, C-7), 111.14 (s, C-3a), 49.14 (s, C-3′), 44.55 (s, C-2″), 31.57 (s, C-5′), 22.94 (s, C-4′); HRMS m/z (EI⁻) [Found: 363.1100, C₁₉H₁₅N₄O₄ requires M⁻ 363.1099)]; LRMS m/z (ESI⁻) 363 ([M-H]⁻, 100%); HPLC Retention time 220: 5.6 min, 95.5%; 254: 5.5 min, 100.0%.

### 2-(2,6-dioxopiperidin-3-yl)-4-((2-methylbenzyl)amino)isoindoline-1,3-dione

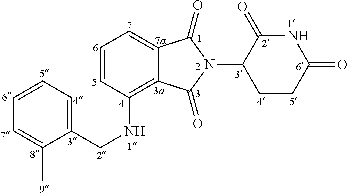 [SMILES: Cc1ccccc1CNc1cccc2c1C(=O)N(C1CCC(=O)NC1=O)C2=O]

2-(2,6-dioxopiperidin-3-yl)-4-fluoroisoindoline-1,3-dione (200 mg, 0.724 mmol, 1.0 eq), 2-methylbenzylamine (180 μL, 1.45 mmol, 2.0 eq) and DIPEA (504.4 μL, 2.90 mmol, 4.0 eq) were dissolved in NMP (4 mL) and heated at 110° C. for 2 h (microwave). The NMP was removed by vigorously blowing nitrogen over the reaction vessel whilst heating to 60° C.. The residual orange oil was purified by flash column chromatography (40-60% EtOAc/petroleum ether) to afford a bright, light yellow solid (107 mg, 39%): R_{f} 0.50 (EtOAc/petroleum ether 1:1); m.p. 202-209° C. (from EtOAc); vₘₐₓ (thin film)/cm⁻¹ 1690 (C═O, s); ¹H NMR (400 MHz, CDCl₃) δ 7.97 (s, 1H, H-1′), 7.48 (dd, J 8.5, 7.1 Hz, 1H), 7.28 (d, J 7.2 Hz, 1H, H-6), 7.24-7.16 (m, 4H, H-4″, 5″, 6″, 7″), 7.12 (d, J 8.5 Hz, 1H, H-7), 6.85 (d, J 8.5 Hz, 1H, H-5), 6.52 (t, J 5.6 Hz, 1H, H-1″), 4.96-4.86 (m, 1H, H-3′), 4.45 (d, J 5.6 Hz, 2H, H-2″), 2.95-2.66 (m, 3H, H-4′, 5′), 2.38 (s, 3H, H-9″), 2.20-2.09 (m, 1H, H-4′, 5′); ¹³C NMR (151 MHz, CDCl₃) δ 170.95 (s, C-6′), 169.58 (s, C-2′), 168.33 (s, C-1), 167.69 (s, C-3), 146.88 (s, C-4), 136.36 (s, C-8″), 136.05 (s, C-3″), 135.27 (s, C-6), 132.64 (s, C-7a), 130.83 (s, C-7″), 127.93 (s, C-4″), 127.57 (s, C-6″), 126.52 (s, C-5″), 117.14 (s, C-5), 112.09 (s, C-7), 110.55 (s, C-3a), 49.06 (s, C-3′), 45.11 (s, C-2″), 31.55 (s, C-5′), 22.96 (s, C-4′), 19.18 (s, C-9″); HRMS m/z (EI⁻) [Found: 376.1306, C₂₁H₁₈N₃O₄ requires M⁻ 376.1303)]; LRMS m/z (ESI⁻) 376 ([M-H]⁻, 100%); HPLC Retention time 220: 10.3 min, 100.0%; 254: 10.3 min, 100.0%.

### 2-(2,6-dioxopiperidin-3-yl)-4-(((4-methylpyridin-3-yl)methyl)amino)isoindoline-1,3-dione

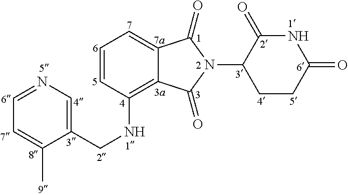 [SMILES: Cc1ccncc1CNc1cccc2c1C(=O)N(C1CCC(=O)NC1=O)C2=O]

2-(2,6-dioxopiperidin-3-yl)-4-fluoroisoindoline-1,3-dione (282 mg, 1.02 mmol, 1.0 eq), (4-methylpyridin-3-yl)methylamine (250 mg, 2.05 mmol, 2.0 eq) and DIPEA (713 μL, 4.09 mmol, 4.0 eq) were dissolved in NMP (4 mL) and heated at 110° C. for 2 h (microwave). The NMP was removed by vigorously blowing nitrogen over the reaction vessel whilst heating to 60° C. The residual orange oil was purified by flash column chromatography (100% EtOAc/petroleum ether) to afford a bright yellow oil. TLC showed this was impure; recolumn (2% MeOH/CH₂Cl₂) yielded a bright yellow/green glass-like oil, which was placed under vacuum for 72 h. Redissolving this in CHCl₃ and removal of solvent via nitrogen stream yielded a bright yellow powder (338 mg, 87%): Rf 0.21 (MeOH/CH₂Cl₂ 1:49); m.p. 104-110° C. (from EtOAc); vₘₐₓ (thin film)/cm⁻¹ 1694 (C═O, s); ¹H NMR (400 MHz, CDCl₃) δ 8.51 (s, 1H, H-1′), 8.50 (s, 1H, H-4″), 8.45 (d, J 4.9 Hz, 1H, H-6″), 7.50 (dd, J 8.5, 7.1 Hz, 1H, H-6′), 7.19-7.10 (m, 2H, H-7, 7″), 6.89 (d, J 8.4 Hz, 1H, H-5), 6.45 (t, J 5.5 Hz, 1H, H-1″), 4.90 (dd, J 12.1, 5.3 Hz, 1H, H-3′), 4.47 (d, J 5.6 Hz, 2H, H-2″), 2.92-2.66 (m, 3H, H-4′, 5′), 2.39 (s, 3H, H-9″), 2.18-2.08 (m, 1H, H-4′, 5′); ¹³C NMR (151 MHz, CDCl₃) δ 170.93 (s, C-6′), 169.55 (s, C-2′), 168.28 (s, C-1), 167.53 (s, C-3), 149.30 (s, C-4″), 148.90 (s, C-6″), 146.38 (s, C-4), 146.27 (s, C-8″), 136.53 (s, C-6″), 132.71 (s, C-3″), 131.42 (s, C-7a), 125.84 (s, C-7″), 116.84 (s, C-5), 112.61 (s, C-7), 111.09 (s, C-3a), 49.12 (s, C-3′), 43.05 (s, C-2″), 31.56 (s, C-5′), 22.94 (s, C-4′), 18.80 (s, C-9″); HRMS m/z (EI⁻) [Found: 377.1253, C₂₀H₁₇N₄O₄ requires M⁻ 377.1255)]; LRMS m/z (ESI⁻) 377 ([M-H]⁻, 100%); HPLC Retention time 220: 6.0 min, 100.0%; 254: 6.1 min, 100.0%.

### 4-(benzyloxy)-2-(2,6-dioxopiperidin-3-yl)isoindoline-1,3-dione

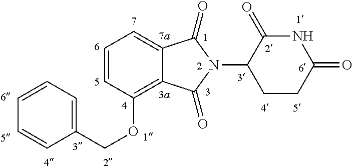 [SMILES: O=C1CCC(N2C(=O)c3cccc(OCc4ccccc4)c3C2=O)C(=O)N1]

2-(2,6-dioxopiperidin-3-yl)-4-hydroxyisoindoline-1,3-dione (100 mg, 0.36 mmol, 1.0 eq) was dissolved in dry DMF (5 mL) under an argon atmosphere. Benzyl bromide (43 μL, 0.36 mmol, 1.0 eq) and potassium carbonate (76 mg, 0.55 mmol, 1.5 eq) were added and the mixture stirred at room temperature for 1.5 h. The reaction mixture was dissolved in 0.5 M LiCl and extracted with EtOAc. The organic layer was washed with water, then brine, then dried with sodium sulfate, filtered and the solvent removed in vacuo. The residue was purified by flash chromatography (5% MeOH/CH₂Cl₂). Recolumn (3% MeOH/CH₂Cl₂) was required as leftover benzyl bromide was still present after first column. This afforded a colourless solid (71 mg, 53.4%): R_{f} 0.65 (MeOH/CH₂Cl₂ 1:9); m.p. 209-211° C. (from CH₂Cl₂); ¹H NMR (400 MHz, DMSO-d₆) δ 11.10 (s, 1H, H-1′), 7.83 (dd, J 8.5, 7.3 Hz, 1H, H-7), 7.60 (d, J 8.5 Hz, 1H, H-6), 7.55-7.30 (m, 6H, H-5, 4″, 5″, 6″), 5.38 (s, 2H, H-2″), 5.09 (dd, J 12.8, 5.4 Hz, 1H, H-3′), 2.88 (ddd, J 17.2, 14.0, 5.3 Hz, 1H, H-4′, 5′), 2.64-2.51 (m, 1H, H-4′, 5′), 2.04 (dp, J 11.4, 3.5 Hz, 1H, H-4′, 5′); LRMS m/z (ESI⁻) 363 ([M-H]⁻, 100%); HPLC Retention time 220: 9.2 min, 98.3%; 254: 9.2 min, 100.0%.

### 2-(2,6-dioxopiperidin-3-yl)-4-(pyridin-3-ylmethoxy)isoindoline-1,3-dione

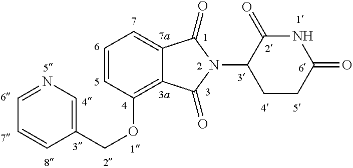 [SMILES: O=C1CCC(N2C(=O)c3cccc(OCc4cccnc4)c3C2=O)C(=O)N1]

2-(2,6-dioxopiperidin-3-yl)-4-hydroxy-1H-isoindole-1,3(2H)-dione (100 mg, 0.36 mmol, 1.0 eq) was dissolved in DMF (5 mL). Potassium carbonate (76 mg, 0.55 mmol, 1.5 eq) was then added and mixed. 3-(bromomethyl)pyridine hydrobromide (82 mg, 0.32 mmol, 0.9 eq) was then added and the mixture stirred at room temperature for 1.5 h. The DMF was removed by vigorously blowing nitrogen over the reaction vessel, and the residue was purified by flash chromatography (2% MeOH/CH₂Cl₂) to afford a colourless solid (68.8 mg, 59%): R_{f} 0.38 (MeOH/CH₂Cl₂ 1:9); m.p. 215-223° C. (from CH₂Cl₂); vₘₐₓ (thin film)/cm⁻¹ 1709 (C═O, s); ¹H NMR (400 MHz, DMSO-d₆) δ 11.12 (s, 1H, H-1′), 8.73 (s, 1H, H-4″), 8.57 (d, J 4.8 Hz, 1H, H-6″), 7.93 (d, J 6.8 Hz, 1H, H-8″), 7.86 (t, J 7.7 Hz, 1H, H-6), 7.64 (d, J 8.6 Hz, 1H, H-7), 7.50 (d, J 8.6 Hz 1H, H-5), 7.46 (dd, J 6.8, 4.8 Hz, 1H, H-7″), 5.42 (s, 1H, H-2″), 5.09 (dd, J 12.9, 5.2 Hz, 1H, H-3′), 2.88 (t, J 15.6 Hz, 1H, H-4′, 5′), 2.65-2.52 (m, 2H, H-4′, 5′), 2.07-1.99 (m, 1H, H-4′, 5′); ¹³C NMR (151 MHz, DMSO-d₆) δ 172.74 (s, C-6′), 169.88 (s, C-2′), 166.74 (s, C-3), 165.30 (s, C-1), 155.27 (s, C-4), 149.29 (s, C-4″), 148.69 (s, C-6″), 137.09 (s, C-8″), 135.25 (s, C-6), 133.29 (s, C-7a), 131.82 (s, C-3″), 123.63 (s, C-7″), 120.21 (s, C-7), 116.75 (s, C-5), 115.79 (s, C-3a), 67.92 (s, C-2″), 48.78 (s, C-3′), 30.92 (s, C-5′), 21.96 (s, C-4′); HRMS m/z (EI⁻) [Found: 364.0940, C₁₉H₁₄N₃O₅ requires M⁻ 364.0939)]; LRMS m/z (ESI⁻) 364 ([M-H]⁻, 100%); HPLC Retention time 220: 5.6 min, 96.9%; 254: 5.7 min, 100.0%.

### 2-(2,6-dioxopiperidin-3-yl)-4-((2-methylbenzyl)oxy)isoindoline-1,3-dione

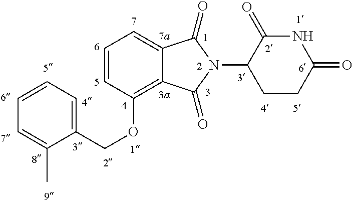 [SMILES: Cc1ccccc1COc1cccc2c1C(=O)N(C1CCC(=O)NC1=O)C2=O]

2-(2,6-dioxopiperidin-3-yl)-4-hydroxy-1H-isoindole-1,3(2H)-dione (100 mg, 0.36 mmol, 1.0 eq) was dissolved in DMF (5 mL). Potassium carbonate (76 mg, 0.55 mmol, 1.5 eq) was then added and mixed. 2-methylbenzyl bromide (44 μL, 0.32 mmol, 0.9 eq) was then added and the mixture stirred at room temperature for 1.5 h. The DMF was removed by vigorously blowing nitrogen over the reaction vessel, and the residue was purified by flash chromatography (2-5% MeOH/CH₂Cl₂) to afford a colourless solid (117 mg, 97%): R_{f} 0.56 (MeOH/CH₂Cl₂ 1:9); m.p. 203-207° C. (from CH₂Cl₂); vₘₐₓ (thin film)/cm⁻¹ 1704 (C═O, s); ¹H NMR (400 MHz, DMSO-d₆) δ 11.12 (s, 1H, H-1′), 7.85 (dd, J 8.5, 7.2 Hz, 1H, H-6), 7.68 (d, J 8.6 Hz, 1H, H-7), 7.55 (d, J 7.0 Hz, 1H, H-4″, 5″, 6″, 7″), 7.48 (d, J 7.2 Hz, 1H, H-5), 7.31-7.18 (m, 3H, H-4″, 5″, 6″, 7″), 5.35 (s, 2H, H-2″), 5.09 (dd, J 12.8, 5.4 Hz, 1H, H-3′), 2.88 (ddd, J 17.0, 13.7, 5.3 Hz, 1H, H-4′, 5′), 2.63-2.51 (m, 2H, H-4′, 5′), 2.36 (s, 3H, H-9″), 2.08-1.99 (m, 1H, H-4′, 5′); ¹³C NMR (151 MHz, DMSO-d₆) δ 172.74 (s, C-6′), 169.90 (s, C-2′), 166.79 (s, C-3), 165.32 (s, C-1), 155.52 (s, C-4), 137.00 (s, C-6″), 136.54 (s, C-8″), 134.08 (s, C-3″), 133.29 (s, C-7a), 130.13 (s, C-7″), 128.16 (s, C-6″), 128.03 (s, C-4″), 125.76 (s, C-5″), 120.23 (s, C-7), 116.58 (s, C-3a), 115.49 (s, C-5), 68.84 (s, C-2″), 48.76 (s, C-3′), 30.93 (s, C-5′), 21.97 (s, C-4′), 18.48 (s, C-9″); HRMS m/z (EI⁻) [Found: 377.1144, C₂₁H₁₇N₂O₅ requires M⁻ 377.1143)]; LRMS m/z (ESI⁻) 377 ([M-H]⁻, 100%); HPLC Retention time 220: 9.8 min, 100.0%; 254: 9.8 min, 100.0%.

### 4-(benzhydrylamino)-2-(2,6-dioxopiperidin-3-yl)isoindoline-1,3-dione

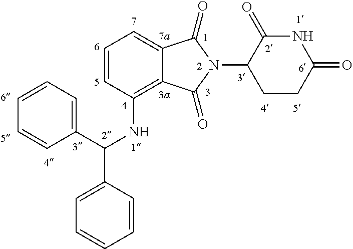 [SMILES: O=C1CCC(N2C(=O)c3cccc(NC(c4ccccc4)c4ccccc4)c3C2=O)C(=O)N1]

2-(2,6-dioxopiperidin-3-yl)-4-fluoroisoindoline-1,3-dione (100 mg, 0.36 mmol, 1.0 eq), benzhydrylamine (250 μL, 1.45 mmol, 4.0 eq) and DIPEA (252. μL, 1.45 mmol, 4.0 eq) were dissolved in NMP (4 mL) and heated at 110° C. for 20 h. The NMP was removed by vigorously blowing nitrogen over the reaction vessel whilst heating to 60° C.. The residual dark brown oil was purified by flash column chromatography (50% EtOAc/petroleum ether) from which an impure product was gained; recolumn (30-40% EtOAc/petroleum ether) yields a bright yellow powder (22 mg, 14%): R_{f} 0.36 (EtOAc/petroleum ether 1:1); m.p. 86-88° C. (from EtOAc); vₘₐₓ(thin film)/cm⁻¹ 1701 (C═O, s); ¹H NMR (600 MHz, CDCl₃) δ 7.92 (s, 1H, H-1′), 7.41-7.27 (m, 11H, H-6, 4″, 5″, 6″), 7.12 (d, J 8.5 Hz, 1H, H-7), 6.86 (d, J 5.5 Hz, 1H, H-1″), 6.71 (d, J 8.5 Hz, 1H, H-5), 5.69 (d, J 5.4 Hz, 1H, H-2″), 4.92 (dd, J 12.5, 5.4 Hz, 1H, H-3′), 2.92-2.68 (m, 3H, H-4′, 5′), 2.13 (dd, J 12.4, 6.4 Hz, 1H, H-4′, 5′); ¹³C NMR (151 MHz, CDCl₃) δ 170.81 (s, C-6′), 169.60 (s, C-2′), 168.27 (s, C-3), 167.61 (s, C-1), 145.87 (s, C-4), 141.44 (s, C-3″), 141.41 (s, C-3″), 136.22 (s, C-6), 132.47 (s, C-7a), 129.22 (s, C-5″), 129.21 (s, C-5″), 128.06 (s, C-6″), 128.03 (s, C-6″), 127.35 (s, C-4″), 127.31 (s, C-4″), 118.25 (s, C-5), 112.43 (s, C-7), 110.96 (s, C-3a), 61.94 (s, C-2″), 49.08 (s, C-3′), 31.55 (s, C-5′), 22.97 (s, C-4′); HRMS m/z (EI⁻) [Found: 438.1460, C₂₆H₂₀N₃O₄ requires M-438.1459)]; LRMS m/z (ESI⁻) 438 ([M-H]⁻, 100%); HPLC Retention time 220: 11.1 min, 100.0%; 254: 11.1 min, 100%.

### 4-(benzhydryloxy)-2-(2,6-dioxopiperidin-3-yl)isoindoline-1,3-dione

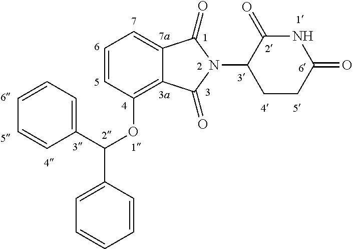 [SMILES: O=C1CCC(N2C(=O)c3cccc(OC(c4ccccc4)c4ccccc4)c3C2=O)C(=O)N1]

2-(2,6-dioxopiperidin-3-yl)-4-hydroxyisoindoline-1,3-dione (100 mg, 0.36 mmol, 1.0 eq) was dissolved in dry DMF (5 mL) under an argon atmosphere. Benzhydryl bromide (89 mg, 0.36 mmol, 1.0 eq) and potassium carbonate (76 mg, 0.550 mmol, 1.5 eq) were added and the mixture stirred at room temperature for 1.5 h. The DMF was then removed by vigorously blowing nitrogen over the reaction vessel. The crude product was purified by flash chromatography (2% MeOH/CH₂Cl₂) to afford a colourless solid (100 mg, 62%): R_{f} 0.53 (MeOH/CH₂Cl₂ 1:9); m.p. 198-208° C. (from CH₂Cl₂); vₘₐₓ (thin film)/cm⁻¹ 1713 (C═O, s); ¹H NMR (400 MHz, DMSO-d₆) δ 11.15 (s, 1H, H-1′), 7.72 (dd, J 8.5, 7.3 Hz, 1H, H-6), 7.63-7.56 (m, 4H, H-4″), 7.50 (d, J 8.6 Hz, 1H, H-7), 7.42 (d, J 7.3 Hz, 1H, H-5), 7.37 (td, J 7.7, 1.5 Hz, 4H, H-5″), 7.27 (td, J 7.3, 1.4 Hz, 2H, H-6″), 6.90 (s, 1H, H-2″), 5.14 (dd, J 12.9, 5.4 Hz, 1H, H-3′), 2.90 (ddd, J 17.1, 13.9, 5.4 Hz, 1H, H-4′, 5′), 2.67-2.51 (m, 2H, H-4′, 5′), 2.07 (ddt, J 13.4, 6.1, 3.6 Hz, 1H, H-4′, 5′); ¹³C NMR (151 MHz, DMSO-d₆) δ 172.77 (s, C-6′), 169.93 (s, C-2′), 166.68 (s, C-3), 165.36 (s, C-1), 154.26 (s, C-4), 140.87 (s, C-3″), 140.85 (s, C-3″), 136.70 (s, C-6), 133.32 (s, C-7a), 128.70 (s, C-5″), 128.68 (s, C-5″), 127.80 (s, C-6″), 127.79 (s, C-6″), 126.17 (s, C-4″), 126.14 (s, C-4″), 121.25 (s, C-′″), 117.38 (s, C-7), 115.71 (s, C-3a), 80.26 (s, C-5), 48.82 (s, C-2″), 40.06 (s, C-3′), 30.96 (s, C-5′), 22.01 (s, C-4′); HRMS m/z (EI⁻) [Found: 439.1300, C₂₆H₁₉N₂O₅ requires M⁻ 439.1299)]; LRMS m/z (ESI⁻) 439 ([M-H]⁻, 100%); HPLC Retention time 220: 10.5 min, 95.9%; 254: 10.5 min, 96.3%.

### 6-(2,6-dioxopiperidin-3-yl)-5H-pyrrolo[3,4-b]pyridine-5,7(6H)-dione

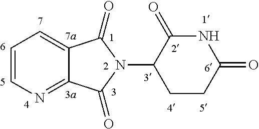 [SMILES: O=C1CCC(N2C(=O)c3cccnc3C2=O)C(=O)N1]

2,3-pyridinedicarboxylic anhydride (201 mg, 1.35 mmol, 1.0 eq), 3-aminopiperidine-2,6-dione hydrochloride (222 mg, 1.35 mmol, 1.0 eq) and NaOAc (166 mg, 2.03 mmol, 1.5 eq) were dissolved in AcOH (5 mL) and heated at 120° C. under reflux for 19 h. The AcOH was removed in vacuo and the residue purified by flash column chromatography (5% MeOH/CH₂Cl₂) to afford a colourless solid (200 mg, 57%): R_{f} 0.50 (MeOH/CH₂Cl₂ 1:9); m.p. 235-245° C. (from CH₂Cl₂); ¹H NMR (400 MHz, DMSO-d₆) δ 11.17 (s, 1H, H-1′), 9.04 (dt, J 5.0, 1.2 Hz, 1H, H-5), 8.42-8.34 (m, 1H, H-7), 7.85 (ddd, J 7.5, 4.8, 0.7 Hz, 1H, H-6), 5.24 (dd, J 12.9, 5.4 Hz, 1H, H-3′), 2.90 (ddd, J 17.2, 13.8, 5.3 Hz, 1H, H-4′, 5′), 2.67-2.52 (m, 2H, H-4′, 5′), 2.08 (dtd, J 13.1, 5.2, 2.2 Hz, 1H, H-4′, 5′); LRMS m/z (ESI⁻) 258 ([M-H]⁻, 100%); HPLC Retention time 220: 5.1 min, 99.7%; 254: 5.2 min, 100.0%.

### 2-(2,6-dioxopiperidin-3-yl)-1H-pyrrolo[3,4-c]pyridine-1,3(2H)-dione

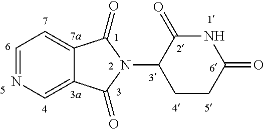 [SMILES: O=C1CCC(N2C(=O)c3ccncc3C2=O)C(=O)N1]

3,4-pyridinedicarboxylic anhydride (201 mg, 1.35 mmol, 1.0 eq), 3-aminopiperidine-2,6-dione hydrochloride (222 mg, 1.35 mmol, 1.0 eq) and NaOAc (166 mg, 2.03 mmol, 1.5 eq) were dissolved in AcOH (5 mL) and heated at 120° C. under reflux for 19.5 h. The AcOH was removed in vacuo and the residue purified by flash column chromatography (5% MeOH/CH₂Cl₂) to afford a colourless solid (314.3 mg, 90%): R_{f} 0.61 (MeOH/CH₂Cl₂ 1:9); m.p. 217-219° C. (from CH₂Cl₂); ¹H NMR (400 MHz, DMSO-d₆) δ 11.18 (s, 1H, H-1′), 9.22-9.11 (m, 2H, H-4, 6), 7.97 (dt, J 4.8, 1.1 Hz, 1H, H-7), 5.22 (dd, J 12.9, 5.4 Hz, 1H, H-3′), 2.89 (ddd, J 17.3, 13.9, 5.4 Hz, 1H, H-4′, 5′), 2.67-2.50 (m, 2H, H-4′, 5′), 2.07 (ddq, J 11.1, 6.0, 2.8 Hz, 1H, H-4′, 5′); LRMS m/z (ESI⁻) 258 ([M-H]⁻, 100%); HPLC Retention time 220: 5.2 min, 99.4%; 254: 5.2 min, 100.0%.

### Materials and Methods—Biological Methods

### Cell Culture

Jurkat cells were cultured in a media of RPMI (Gibco), 10% heat-inactivated FBS (Gibco), 1% Glutamax (Gibco), 1% MEM non-essential amino acids (Gibco), 1% sodium pyruvate (Gibco) and 1% PenStrep (Sigma). Lenti-X 293-T cells (Takara) were cultured in a media of DMEM high glucose (Gibco), 10% Tet-free FBS (Takara), 1% sodium pyruvate and 1% PenStrep. HEK293T cells were cultured in a media of DMEM, 10% heat-inactivated FBS, 1% MEM non-essential amino acids and 1% PenStrep. Cells were incubated at 37° C. and 5% CO₂.

### Cloning

The degradation reporter plasmid, pLVX-Sprout, was generated by inserting a gene fragment encoding for IKZF3 130aa-142aa 170-189aa fused to EGFP via a linker into pLVX-EF1a-IRES-mCherry (Takara) using XbaI/BamHI restriction sites. Gene fragments encoding for IKZF3 143aa-169aa sequences were cloned into pLVX-Sprout using the Golden Gate Assembly Kit BsmBI-v2 (New England Biolabs).

Lentivirus Vector Production and Transduction The below method was used to generate lentiviral vectors for plasmids used in initial compound screening (WT and Q147 Å) The following method was also used to generate the library lentiviral vector pool. Lenti-X 293-T cells were seeded into 100 mm TC-treated cell culture dishes (Falcon). When cells were 90% confluent, 7 g transgene plasmid was mixed with Lenti-X™ Packaging Single Shot (VSV-G) (Takara) and incubated for 10 minutes at room temperature before being added to cells. 6 hours later, Viralboost (Alstem) was added at a 500× dilution. After 3 days, the supernatant was harvested and centrifuged at 1000×g for 10 minutes, then supernatant was passed through a 0.22 μm PES filter. Lenti-X concentrator (Takara) was added at a 4× dilution and the supernatant was incubated at 4° C. for 6 hours. Supernatant was centrifuged at 4° C. 1500×g for 45 minutes, supernatant was removed and virus pellet was resuspended at a 50× concentration in cell culture media used for Jurkat cells. Lentivirus vector aliquots were frozen at −80° C. For transductions, lentivirus vectors were added to Jurkat cells in 6 well TC-treated cell culture dishes (Falcon) with 3×10⁶ cells per well and incubated for 2 days at 37° C. and 5% CO₂. After 2 days, the supernatant was removed and replaced with fresh media.

The following method was used to generate lentiviral vectors for plasmids tested in library hit confirmation experiments: WT, QPIS, ANQS, APIS, HEIP, FEVP, WEIK, FDIP, AEVK, WNQS, WEIS, WNIQ, and WDIA. HEK293T cells were seeded into 6 well TC-treated cell culture dishes (Falcon) at 7×10⁵ cells per well and incubated overnight at 37° C. and 5% CO₂. 888 ng of transgene plasmid, 444 ng of psPAX2 (Addgene Cat #12260), and 222 ng of pMD2.G (Addgene Cat #12259) were combined in 100 μL of OptiMEM (Thermo). In a separate tube 2 μL of PEIPro (Polyplus) was added to 100 μL of OptiMEM. The solution containing PEIPro and DNA was mixed and incubated at room temperature for 15 minutes to create the transfection mixture. The transfection mixture was added to cells and incubated for 5-6 hours at 37° C. and 5% CO₂. After 2 days, the supernatant was harvested and fresh media was added to cells. 24 hours later, the supernatant was harvested and combined with the first supernatant harvested. Supernatant was centrifuged at 2000×g room temperature for 5 minutes, then the supernatant was filtered through a 0.45 μm cellulose acetate syringe filter and frozen at −80° C. For transductions, lentivirus vectors were added to Jurkat cells in 6 well TC-treated cell culture dishes (Falcon) with 2.5×10⁶ cells per well in media containing 4 μg/mL polybrene (Sigma). The cells were centrifuged at 500×g for 60 minutes at 30° C. Cells were incubated for 3 days at 37° C. and 5% CO₂ prior to replacing supernatant with fresh media.

### GFP Degradation Flow Cytometry Assay

Test compounds (10 mM stock in DMSO) were either serially diluted 10-fold to create a 6 point dilution spanning 1 nM-100 μM (final assay concentration) or serially diluted 2-fold to create a 12 point dilution spanning 5 nM-10 μM. Jurkat cells were added to 96 well U-bottom assay plates (Falcon) at 1×10⁵ cells per well and treated with compounds for 18 hours at 37° C. and 5% CO₂. Cells were stained with 1 μg/mL DAPI (ThermoFisher) and acquired on a Cytoflex S (Beckman Coulter). Data was analysed using FlowJo 10.8.1 (BD).

### Compound Solubility

5 μL of 10 mM DMSO stock solution was diluted to 100 uL with pH 7.4 PBS, equilibrated for 1 hour at room temperature and filtered through Millipore Multiscreen HTS-PCF filter plates (MSSL BPC). The filtrate was quantified by suitably calibrated Charged Aerosol Detector.

### Protein Expression and Purification

Wild-type human 6×His-TEV-Cereblon and 6×His-TEV-DDB1ΔBPB were cloned in pFastbac-HTb (Genscript), and recombinant proteins were expressed in Super 9 SFX insect cells (SH3A3187.02, Cytiva) using the BacMam baculovirus expression system (ThermoFisher). For protein purification cells were resuspended in buffer containing 500 mM NaCl (Sigma), 10% glycerol (Sigma), 50 mM Tris pH 7.7 (Sigma), 15 mM imidazole (Sigma), 90.5 mM TCEP (Sigma), 5 μL/mL protease inhibitor cocktail (Sigma) and 0.66 μL/mL benzonase (Novagen). Cells were lysed by sonication and pelleted by ultra-centrifugation. The supernatant was passed over Ni-NTA affinity resin (Qiagen) and eluted in buffer containing 500 mM NaCl, 10% glycerol, 50 mM Tris pH 7.7 and 90.5 mM TCEP supplemented with 350 mM imidazole. Proteins were further purified via size exclusion chromatography in 50 mM Tris pH 7.5, 5% glycerol, 2 mM DTT (Sigma), and 150 mM NaCl. Protein fractions were concentrated by ultrafiltration using an Ultra 10 K MWCO device (Amicon), then flash frozen in liquid nitrogen and stored at −80° C.

### CRBN Binding

Test compounds (10 mM stock in DMSO) were serially diluted 4-fold to create an 11 point dilution spanning 0.1 nM-100 μM. 100 nL of compounds were added to black 384 well low volume assay plates (Greiner) using an Echo 555 acoustic dispenser (Labcyte). 5 uL 6×His-TEV-Cereblon/6×His-TEV-DDB1ΔBPB (5 nM final assay concentration) containing Alexa647 labeled lenalidomide (50 nM final assay concentration) were added to the assay plates using a Multidrop Combi dispenser (ThermoFisher) in assay buffer (50 mM HEPES (Sigma), 150 mM NaCl (Sigma), 5% Glycerol (Sigma), 1 mM CHAPS (Sigma) and 1 mM DTT (Sigma) pH 7.4). Assay plates were centrifuged at 1000 rpm for 1 minute and then incubated for 15 minutes at room temperature. 5 μL Eu-W1024-labeled Anti-6×His antibody (1 nM final assay concentration) (Perkin Elmer) in assay buffer was added to all wells, plates were centrifuged at 1000 rpm for 1 minute and then incubated for 30 minutes at room temperature. After excitation of europium fluorescence at 337 nm, emission at 615 nm (donor, europium) and 665 nm (acceptor, Alexa647) were recorded with a 20 μs delay to reduce background fluorescence on an Envision 2104 microplate reader (Perkin Elmer). The data was reported as a 665/615 ratio and normalised between high (no test compound) and low (no protein) controls.

### Endogenous IKZF1 Immunostaining

Test compounds (10 mM stock in DMSO) were serially diluted 10-fold to create a 6 point dilution spanning 1 nM-100 μM (final assay concentration). Jurkat cells were added to 96 well U-bottom assay plates (Falcon) at 1×105 cells per well and treated with compounds for 18 hours at 37° C. and 5% CO₂. Following incubation, cells were stained with LIVE/DEAD™ Fixable Aqua Dead Cell Stain (Thermo) diluted 1000× in PBS for 30 minutes at rom temperature and then incubated with Human TruStain FcX block (Biolegend) diluted 40× in PBS+1% FBS (Gibco) for 10 minutes at room temperature. Intranuclear staining of IKZF1 was carried out using the Transcription Factor Buffer Set (BD Bioscience) according to the manufacturer's protocol and staining with IKZF1 AF488 antibody (clone R32-1149, BD Bioscience) diluted 40× for 30 minutes at room temperature. Cells were acquired on a Cytoflex S (Beckman Coulter) and data analysed using FlowJo 10.8.1 9 (BD).

### Pooled Library Construction

A library of 8,380 IKZF3 degrons was cloned into pLVX-Sprout to create the eGFP/mCherry protein degradation reporter plasmid pool. A lentivirus pool was then generated using the Lenti-X lentiviral vector production method.

### Pooled Library Screen

Jurkat cells were transduced at a multiplicity of infection of <0.3 with the lentivirus vector library in order to maximise the number of cells with a single integration. Transductions were carried out in 6 well TC-treated cell culture dishes (Falcon) with 3×10⁶ cells per well. Cells were incubated for 2 days at 37° C. and 5% CO₂ before the supernatant was removed and replaced with fresh media. 6 days post-transduction, mCherry+ cells were isolated via FACS using the FACS Aria Fusion (BD) in a buffer containing PBS (Gibco), 2 mM EDTA (Gibco), 25 mM HEPES (Sigma) and 1% BSA (Sigma). 8 days later, cells were treated with DMSO or a thalidomide analog for 18 hours at 37° C. and 5% CO₂. mCherry+GFPlow and NOT(mCherry+GFPlow) cells were sorted by FACS using the FACS Aria Fusion (BD). DMSO treated cells were used to set the mCherry+GFPlow gate and dead cells were excluded from sorting by staining cells with 1 μg/mL DAPI (ThermoFisher). gDNA was isolated using the Maxwell RSC 48 (Promega). gDNA from cells that were unsorted and untreated were also isolated to determine the library representation on the day of compound treatment. Throughout the screen >600× coverage of the library was maintained. 3 biological replicates were carried out with a new transduction taking place each time.

### Next-Generation Sequencing (NGS)

A two-step PCR was used where the first PCR (PCR1) amplified the integrated degron sequence from gDNA and the second PCR (PCR2) added Illumina adaptors and barcodes to permit multiplexing. See Supp F for PCR1 and PCR2 primers. Q5 Hot Start High-Fidelity 2× Master Mix (New England Biolabs) was used for both PCRs. Sufficient gDNA was amplified in PCR1 in order to maintain >600× coverage and amplicons were purified using solid phase reversible immobilization (SPRI) beads (Beckman Coulter). Purified PCR1 products were analysed on the Tapestation 4200 (Agilent) to confirm purity prior to proceeding to PCR2. Sufficient PCR1 product was amplified in PCR2 in order to maintain >600× coverage and amplicons were run on a 2% agarose TAE gel. Target bands were excised and purified using the NucleoSpin Gel and PCR Clean-up Kit (Machery Nagel). Amplicons were analysed on the Tapestation 4200 and quantified using Qubit dsDNA HS kit (ThermoFisher). PCR products were pooled and sequenced as paired-reads on the NovaSeq (Illumina) using a SP2 200 cycles kit (Illumina).

### NGS Data Analysis

Sample fastq files were processed to align reads to reference degron sequences using the alignment tool Vsearch (https://pubmed.ncbi.nlm.nih.gov/27781170/) in order to count the number of times each degron was present within each sample. Count distributions, percentage of read mapping, library coverage and replicate correlation per sample were assessed for QC purposes. Counts for each degron were then subject to Limma Voom linear regression analysis (https://genomebiology.biomedcentral.com/articles/10.1186/gb-2014-15-2-r29) to generate an estimated log fold change between replicate samples in the positively (mCherry+GFPlow) sorted fraction vs replicate samples in the negatively (NOT(mCherry+GFPlow) sorted fraction for each degron sequence.

### Immunoblotting

Test compounds (10 mM stock in DMSO) were serially diluted 10-fold to create a 3 or 6 point dilution spanning 100 nM-10 μM or 1 nM-10 μM respectively (final assay concentration). Jurkat cells were added to T25 flasks (Falcon) at 2×10⁶ cells per flask and were incubated with compounds for 18 hours at 37° C. and 5% CO₂. Cells were lysed in buffer containing 9 M urea (Sigma), 75 mM Tris-HCl (Sigma), 0.15 M β-mercaptoethanol (Merck) and 10× diluted protease inhibitor cocktail (Merck). Cells lysates were sonicated for 10 seconds 3 times, centrifuged at 13000 rpm for 10 minutes at 4° C. and the supernatants were transferred into fresh tubes. Protein concentrations were measured using a Pierce BCA Protein Assay Kit (Thermo). Proteins were boiled in Laemmli sample buffer (Bio-Rad) for 5 minutes at 100° C. and separated by SDS-PAGE (7.5% Mini-PROTEAN TGX gels, Bio-Rad) and compared to the PageRuler Prestained Protein Ladder (Thermo). Semi-dry blotting was done on a Trans-Blot Turbo Transfer System (Bio-Rad) with the Trans-Blot Turbo RTA Mini 0.2 μm PVDF Transfer Kit (Bio-Rad) using the default mixed molecular weight mode. Membranes were blocked in 5% non-fat dry milk (Bio Rad) in TBS-T. Primary IKZF3 (D1C1E, Cell Signaling Technology Europe), eGFP (F56-6A1.2.3, Thermo) and vinculin (E1E9V, Cell Signaling Technology Europe and sc-25336, Santa Cruz Biotechnologies) antibodies were diluted 1000×, 500× and 50× respectively in 5% non-fat dry milk in TBS-T. Membranes were incubated with primary antibodies overnight at 4° C., washed with TBS-T 3 times, incubated with HRP conjugated secondary antibodies (Promega) for 1 hour at room temperature, washed with TBS-T 3 times and imaged with Clarity Western ECL substrate (Bio-Rad). Membranes were scanned with a Bio-Rad ChemiDoc XRS+ Imaging System (Bio-Rad) and signal intensities were quantified using Image Lab software (Bio Rad). Band densitometry was assessed, normalized to vinculin bands, and reported as percentage of the DMSO control lane.

### CRISPR Editing

Jurkat cells were edited 8 days after lentiviral transduction. For each gene, 3 sgRNAs targeting a single exon were designed using the Synthego CRISPR Design tool. See Supp F for sgRNA sequences. sgRNA were resuspended to 100 M in IDT duplex buffer and then combined to create pools of sgRNA. Cas9 RNPs were prepared by combining 2.5 μL 36.6 μM Alt-R® S.p. Cas9 Nuclease V3 (IDT) and 2 μL 100 μM sgRNA pool in IDT duplex buffer and incubating for 10 minutes at room temperature. 1.5×10⁶ cells in 20 μL SE buffer (Lonza) were mixed with the Cas9 RNPs and then electroporated using a 4D-Nucleofector (Lonza) on pulse code CL-120. Estimation of knockout efficiency was performed by PCR amplification of the sgRNA target site using primers described in Supp F. PCR products were Sanger sequenced and editing efficiency was assessed using the online ICE webtool (https://ice.synthego.com).

### Materials and Methods—Plasmid Information

Plasmid: pLVX-Sprout
Backbone: pLVX-EF 1α-IRES-mCherry (Takara)
Insert sequence cloned into MCS:

cagtgttctagaggaatagccaccatgtccggattcaatgtcttaatg
gttcataagcgaagccatactggtgaagagacggacgtctcagaaaaa
ccttttaagtgtcacctctgcaactatgcatgccaaagaagagatgcg
ctcacgcgtgctgaagctgctgcaaaggaagctgcagctaaggaggct
gcagctaaggctgtgagcaagggcgaggagctgttcaccggggtggtg
cccatcctggtcgagctggacggcgacgtaaacggccacaagttcagc
gtgtccggcgagggcgagggcgatgccacctatggcaaactgaccctg
aaattcatctgcaccaccggcaaactgcccgtgccctggcccaccctc
gtgaccaccctgacctatggcgtgcagtgcttcagccgctatcccgac
cacatgaaacagcacgacttcttcaagtccgccatgcccgaaggctac
gtccaggagcgcaccatcttcttcaaggacgacggcaactacaagacc
cgcgccgaggtgaagttcgagggcgacaccctggtgaaccgcatcgag
ctgaagggcatcgacttcaaggaggacggcaacatcctggggcacaag
ctggagtacaactacaacagccacaacgtctatatcatggccgacaag
cagaagaacggcatcaaggtgaacttcaagatccgccacaacatcgag
gacggcagcgtgcagctcgccgaccactatcagcagaacacccccatc
ggcgacggccccgtgctgctgcccgacaaccactatctgagcacccag
tccgccctgagcaaagaccccaacgagaaacgcgatcacatggtcctg
ctggagttcgtgaccgccgccgggatcactctcggcatggatgaactg
tataaataaggatccccacat

Plasmid map:
Plasmid: pLVX-Sprout-IKZF3 130-189aa (WT)
Backbone: pLVX-Sprout
Insert sequence cloned into pLVX-Sprout:

cagtgtgggctaccgtctcagtgaacgcccattccagtgtaatcagtg
tggggcatcttttactcagaaaggtaacctcctccgccacattaaact
gcacacaggggaaacgagacggtagccgccacat

Plasmid map:
Plasmid: pLVX-Sprout-IKZF3 130-189aa Q147A (ANQS)
Backbone: pLVX-Sprout
Insert sequence cloned into pLVX-Sprout:

cagtgtgggctaccgtctcagtgaacgcccattcgcctgtaatcagtgtgg
ggcatcttttactcagaaaggtaacctcctccgccacattaaactgcacac
aggggaaacgagacggtagccgccacat

Plasmid: pLVX-Sprout-IKZF3 130-189aa Q147A N149P Q150I (APIS)
Backbone: pLVX-Sprout
Insert sequence cloned into pLVX-Sprout:

cagtgtgggctaccgtctcagtgaacgcccattcgcctgtccaatctgtgg
ggcatcttttactcagaaaggtaacctcctccgccacattaaactgcacac
aggggaaacgagacggtagccgccacat

Plasmid: pLVX-Sprout-IKZF3 130-189aa Q147W (WNQS)
Backbone: pLVX-Sprout
Insert sequence cloned into LVX-S rout: PGP-85 DNA

cagtgtgggctaccgtctcagtgaacgcccattctggtgtaatcagtgtgg
ggcatcttttactcagaaaggtaacctcctccgccacattaaactgcacac
aggggaaacgagacggtagccgccacat

Plasmid: pLVX-Sprout-IKZF3 130-189aa Q147H N149E Q150I S154P (HEIP)
Backbone: pLVX-Sprout
Insert sequence cloned into pLVX-Sprout:

cagtgtgggctaccgtctcagtgaacgcccattccattgtgagatctgtgg
ggcaccatttactcagaaaggtaacctcctccgccacattaaactgcacac
aggggaaacgagacggtagccgccacat

Plasmid: pLVX-Sprout-IKZF3 130-189aa Q147F N149E Q150V S154P (FEVP)
Backbone: pLVX-Sprout
Insert sequence cloned into pLVX-Sprout:

cagtgtgggctaccgtctcagtgaacgcccattcttctgtgaggtgtgtgg
ggcaccctttactcagaaaggtaacctcctccgccacattaaactgcacac
aggggaaacgagacggtagccgccacat

Plasmid: pLVX-Sprout-IKZF3 130-189aa Q147W N149E Q150I S154K (WEIK)
Backbone: pLVX-Sprout
Insert sequence cloned into pLVX-Sprout:

cagtgtgggctaccgtctcagtgaacgcccattctggtgtgaaatctgtgg
ggcaaagtttactcagaaaggtaacctcctccgccacattaaactgcacac
aggggaaacgagacggtagccgccacat

Plasmid: pLVX-Sprout-IKZF3 130-189aa Q147F N149D Q150I S154P (FDIP)
Backbone: pLVX-Sprout
Insert sequence cloned into pLVX-Sprout:

cagtgtgggctaccgtctcagtgaacgcccattcttctgtgacatatgtgg
ggcaccctttactcagaaaggtaacctcctccgccacattaaactgcacac
aggggaaacgagacggtagccgccacat

Plasmid: pLVX-Sprout-IKZF3 130-189aa Q147A N149E Q150V S154K (AEVK)
Backbone: pLVX-Sprout
Insert sequence cloned into pLVX-Sprout:

cagtgtgggctaccgtctcagtgaacgcccattcgcttgtgaagtgtgtgg
ggcaaaatttactcagaaaggtaacctcctccgccacattaaactgcacac
aggggaaacgagacggtagccgccacat

Plasmid: pLVX-Sprout-IKZF3 130-189aa Q147W N149E Q150I (WEIS)
Backbone: pLVX-Sprout
Insert sequence cloned into pLVX-Sprout: PGP-91 DNA

cagtgtgggctaccgtctcagtgaacgcccattctggtgtgagatttgtgg
ggcatcttttactcagaaaggtaacctcctccgccacattaaactgcacac
aggggaaacgagacggtagccgccacat

Plasmid: pLVX-Sprout-IKZF3 130-189aa Q147W Q150I S154Q (WNIQ)
Backbone: pLVX-Sprout
Insert sequence cloned into pLVX-Sprout:

cagtgtgggctaccgtctcagtgaacgcccattctggtgtaacatctgtgg
ggcacagtttactcagaaaggtaacctcctccgccacattaaactgcacac
aggggaaacgagacggtagccgccacat

Plasmid: pLVX-Sprout-IKZF3 130-189aa Q147W N149D Q150I S154A (WDIA)
Backbone: pLVX-Sprout
Insert sequence cloned into pLVX-Sprout:

cagtgtgggctaccgtctcagtgaacgcccattctggtgtgatatctgtgg
ggcagcctttactcagaaaggtaacctcctccgccacattaaactgcacac
aggggaaacgagacggtagccgccacat

Plasmid: pLVX-Sprout-IKZF3 130-189aa N149P Q150I (QPIS)
Backbone: pLVX-Sprout
Insert sequence cloned into pLVX-Sprout:

cagtgtgggctaccgtctcagtgaacgcccattccagtgtcctatctgtgg
ggcaagctttactcagaaaggtaacctcctccgccacattaaactgcacac
aggggaaacgagacggtagccgccacat

### Materials and Methods—Computational Experimentation

Docking was carried out using the crystal structure DDB1-CRBN-pomalidomide complex bound to IKZF1(ZF2) from the Protein Data Bank (PDB ID: 6h0f) as a template.

Protein energetics and mutation evaluation were carried out using the PositionScan function in FoldX 5. The 3D coordinate files generated for the mutant proteins were then used for subsequent docking.

Docking was carried out using GOLD version 2020.1 from the Cambridge Crystallographic Data Centre. The position of the original pomalidomide ligand in the template crystal structure was used to define the binding pocket for new ligands using GOLD's ‘cavity’ functionality. The imide ring region of the original pomalidomide ligand was used to specify the position onto which the imide region of screened ligands should be superimposed using GOLD's ‘scaffold’ functionality. All parameters were kept as default during docking screens, except constraint weight, which was increased from the default value of 5 to 10. For every ligand/mutant combination in the large scale high throughput screens, 10 poses were generated, from which the highest ranked was taken forward for further analysis. 10 poses were also used in the validation screen study. In the smaller scale screen of benzyl pomalidomide variants, a constraint weight of 20 was used, and 100 poses were generated, from which the highest ranked was taken forward for further analysis.

Energetic evaluation of docked poses produced by GOLD was carried out using AutoDock4.

Mutational analysis for use in the lentiviral library screen was performed using the PositionScan functionality in foldx and the PositionMutation functionality in Rosetta. The same crystal structure (6h0f) was used with the pomalidomide ligand removed.

### Results

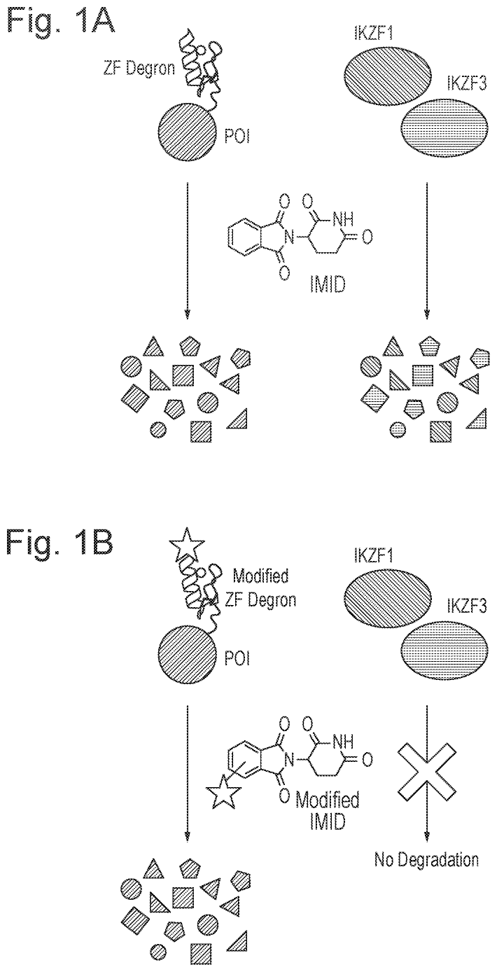

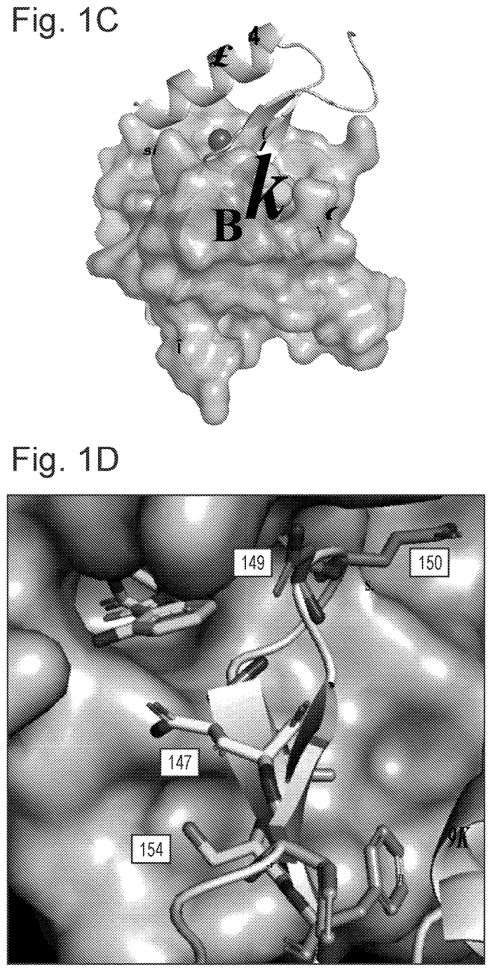

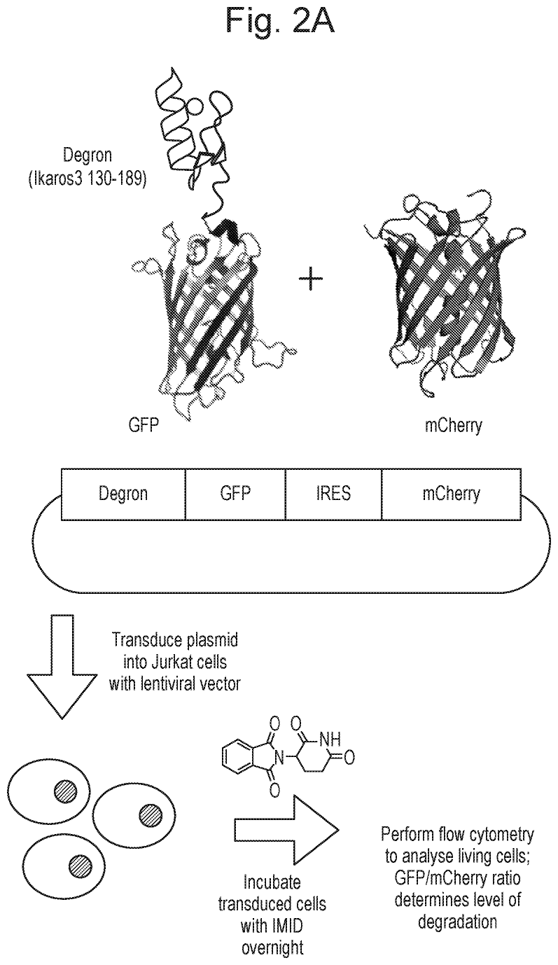

Our initial assessment of the IMID/ZF system was based on the crystal structure 6HOF (FIG. 1B). Inspection of the CRBN-pomalidomide-ZF interface suggested that Q147 was the only suitable candidate for bump-and-hole mutation, as no other residues in the ZF had side chains proximal to the phthalimide half off the ligand protruding from CRBN. The conserved glycine at position 152 mentioned previously is in contact with the ligand, but cannot be mutated into anything smaller. Residues 149, 150 and 151 are also proximal to this region of pomalidomide, but via their backbones instead of their side chains, again making them unsuitable for bump- and hole.

Having decided to focus our attention on residue 147, an initial in silico constrained docking screen was carried out to study both the best potential mutations to make at residue 147, and the best potential chemical modifications to make to the IMID molecule. Firstly Foldx's PositionScan functionality was used to produce reasonable poses for the CRBN-ZF duplex (in absence of the pomalidomide ligand) upon mutation of Q147 to all other residue types—this also allowed nearby residues to change conformation if energetically favourable. Subsequently GOLD was used to dock a library of virtual IMID analogues —‘drawn’ in PyMOL off the crystal ligand coordinates using the ‘builder’ functionality—into the empty different mutant CRBN-ZF duplexes.

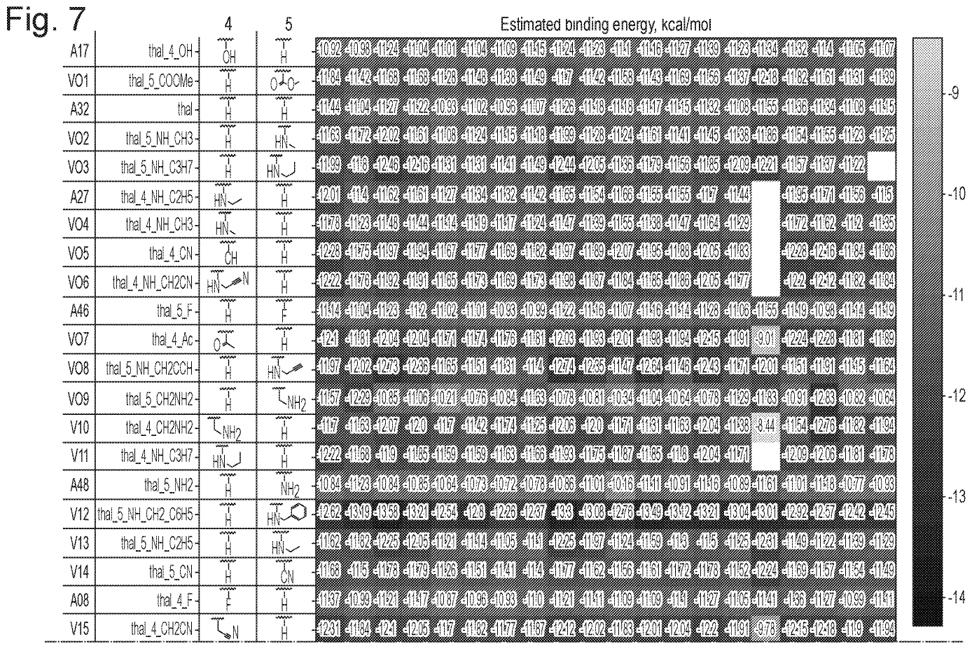

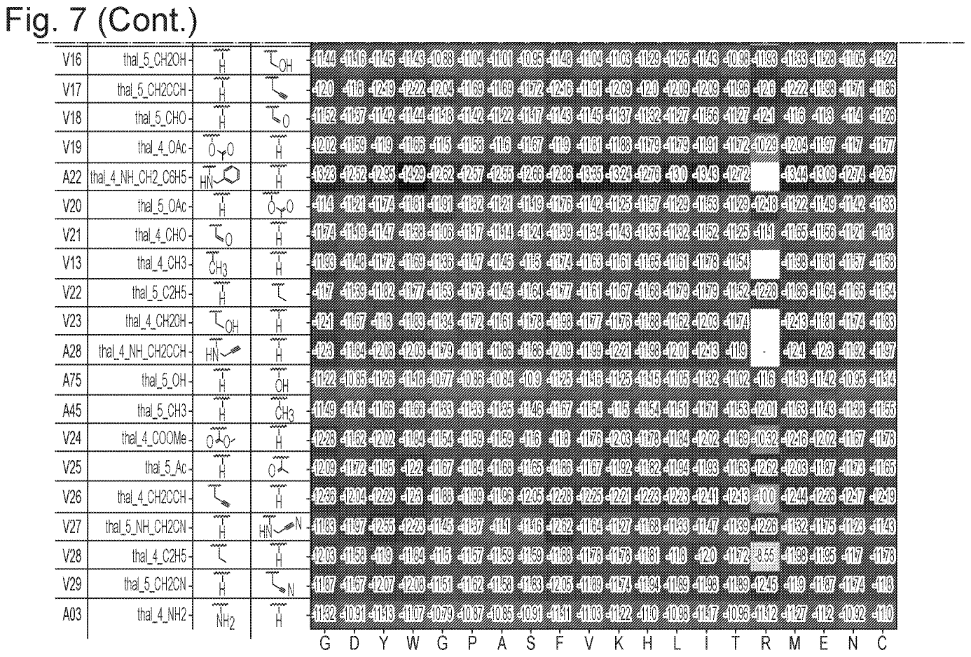

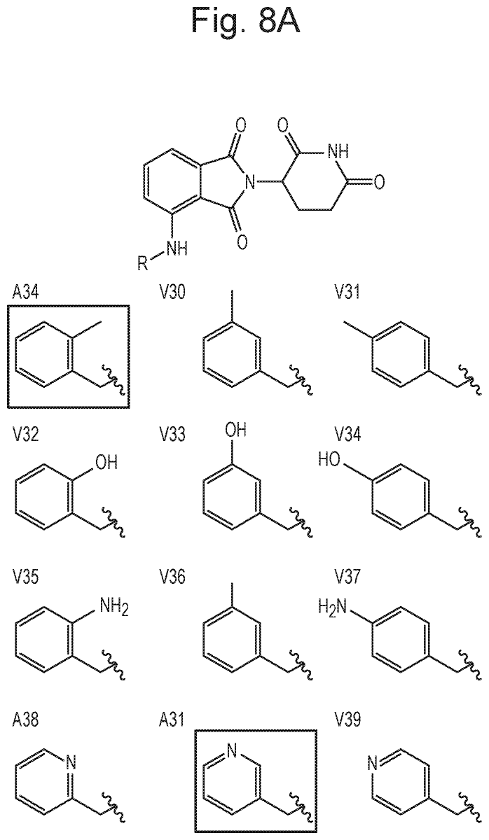

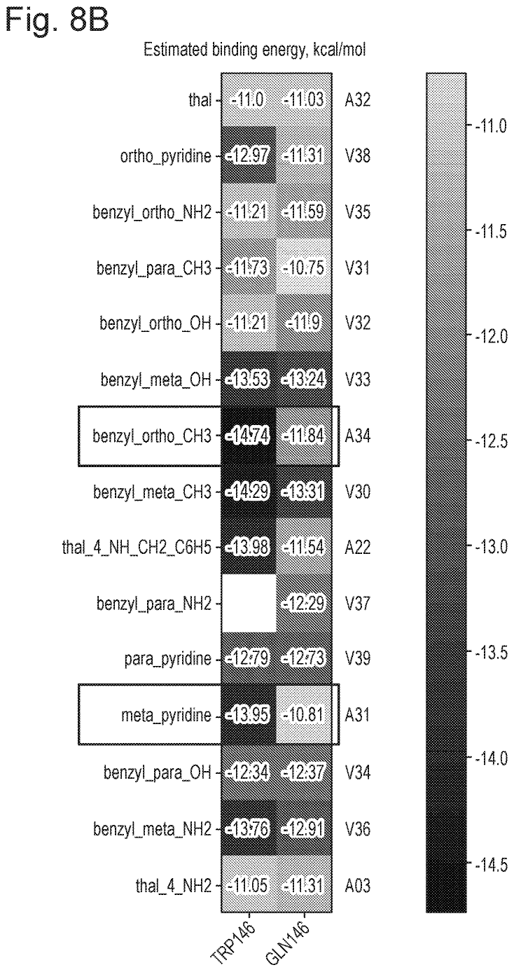

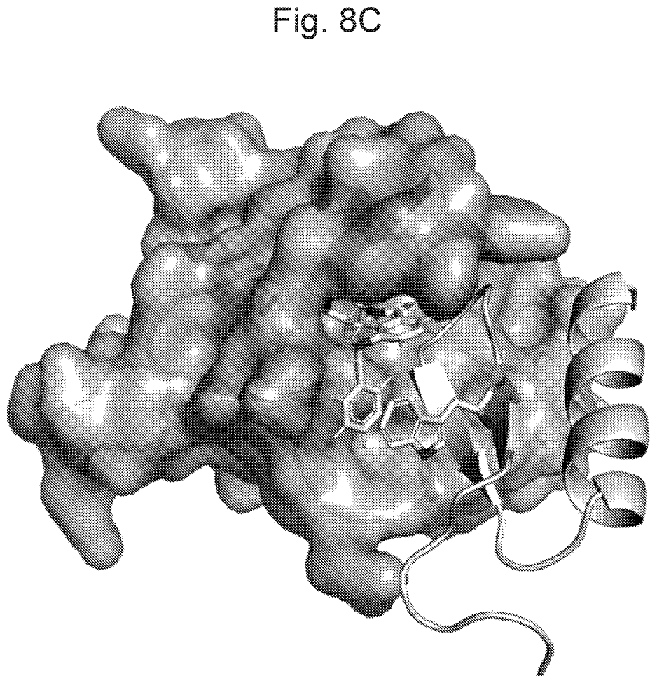

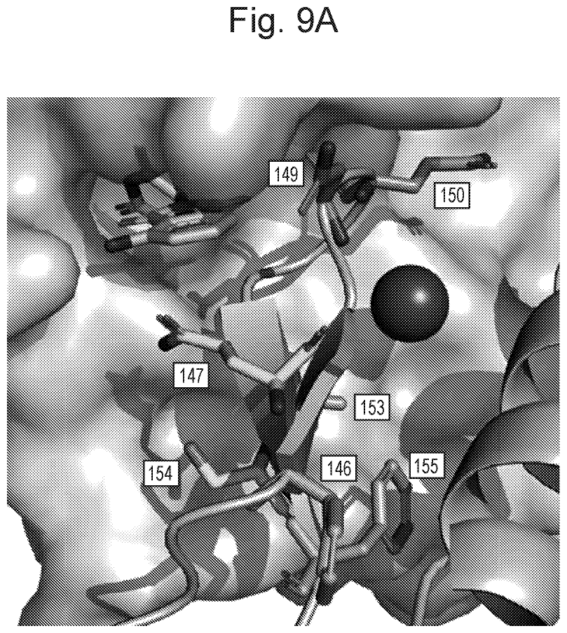

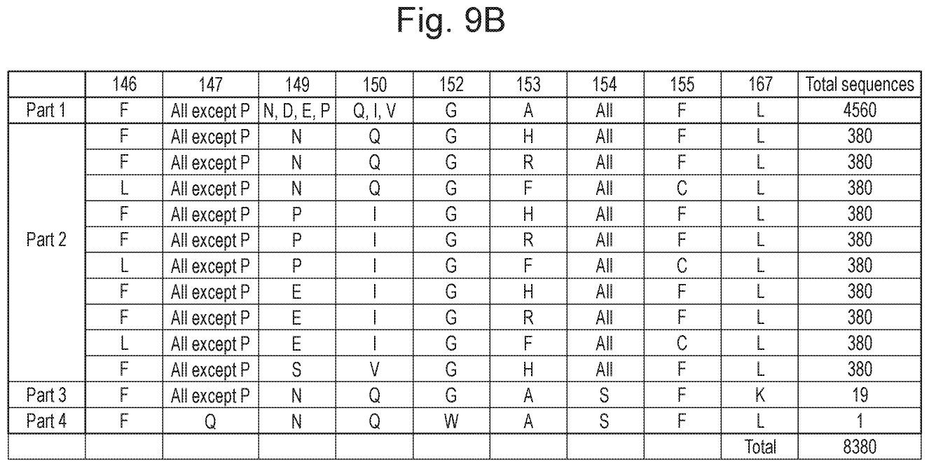

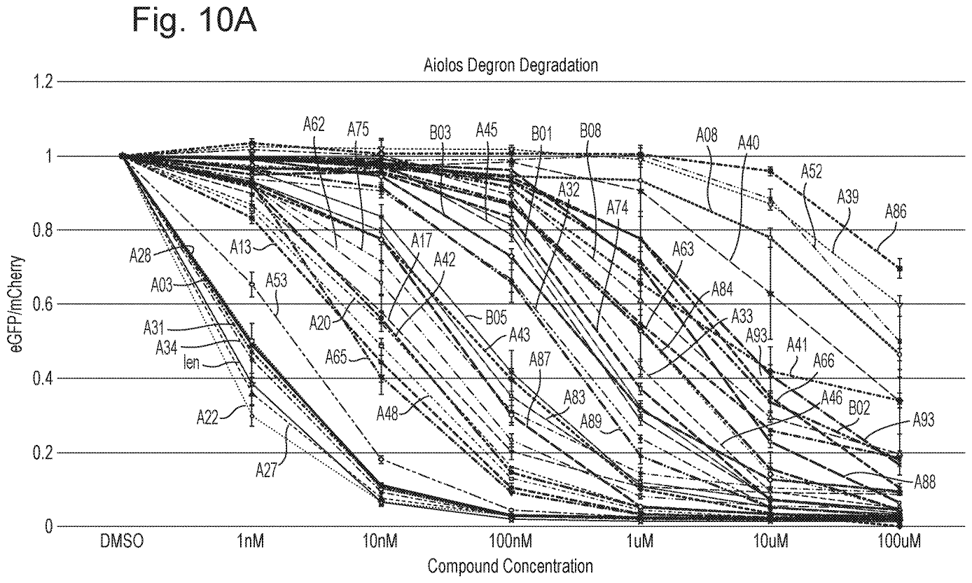

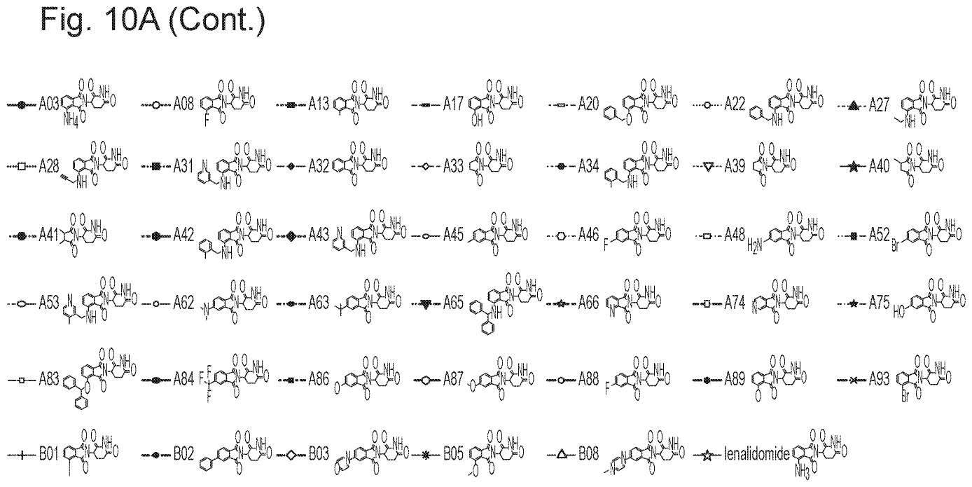

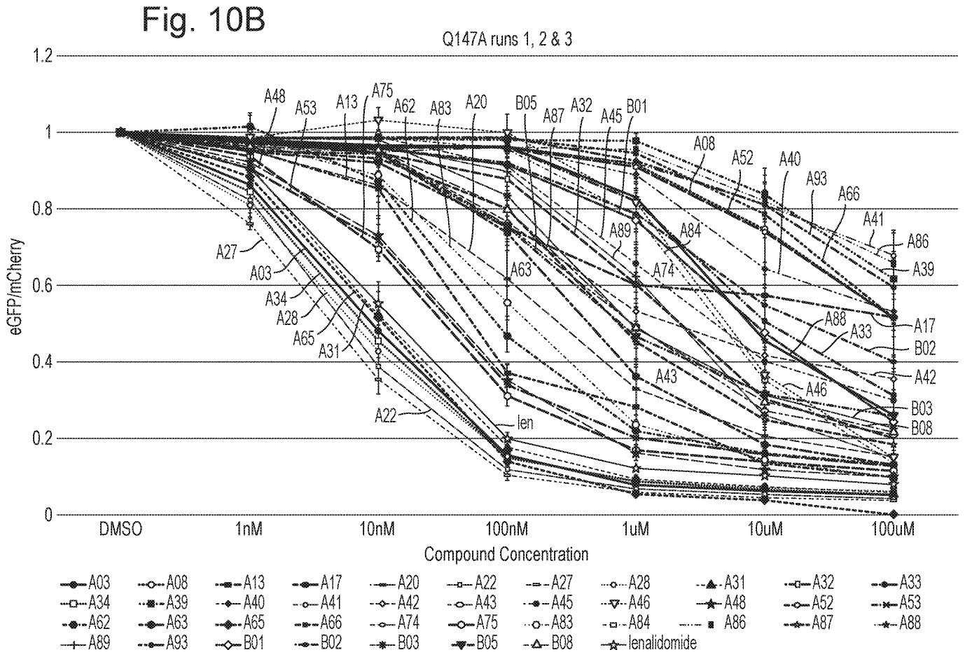

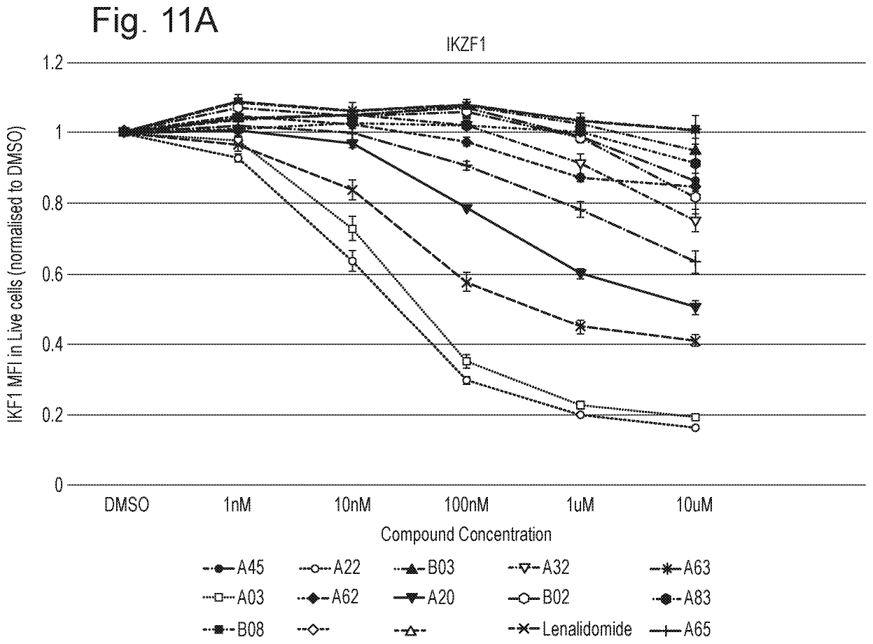

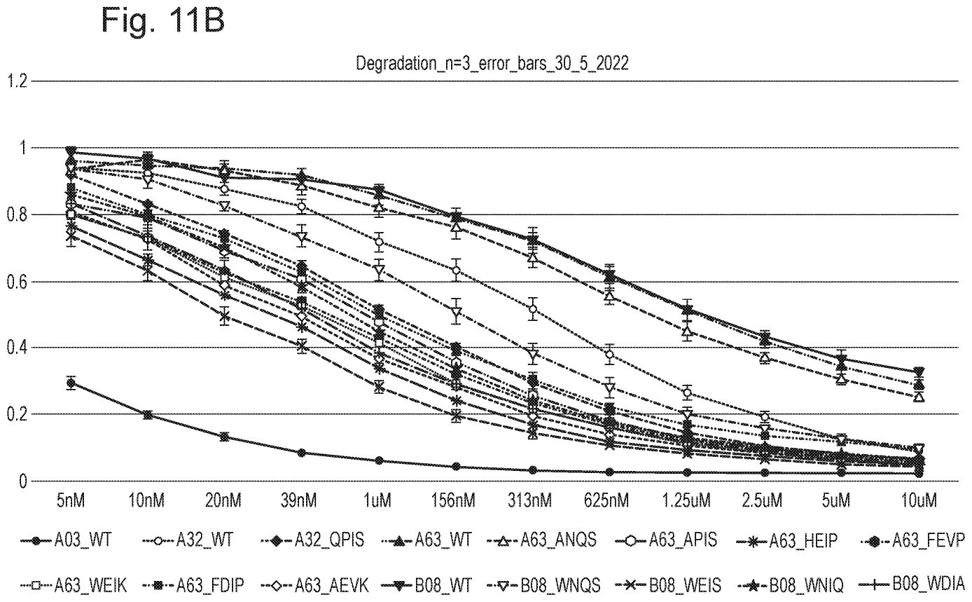

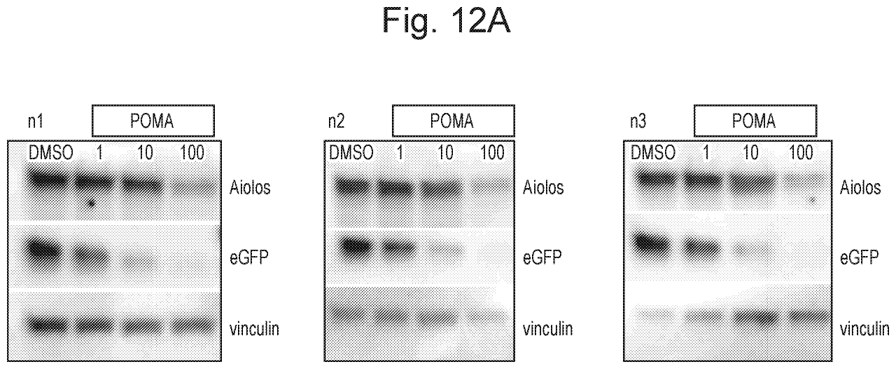

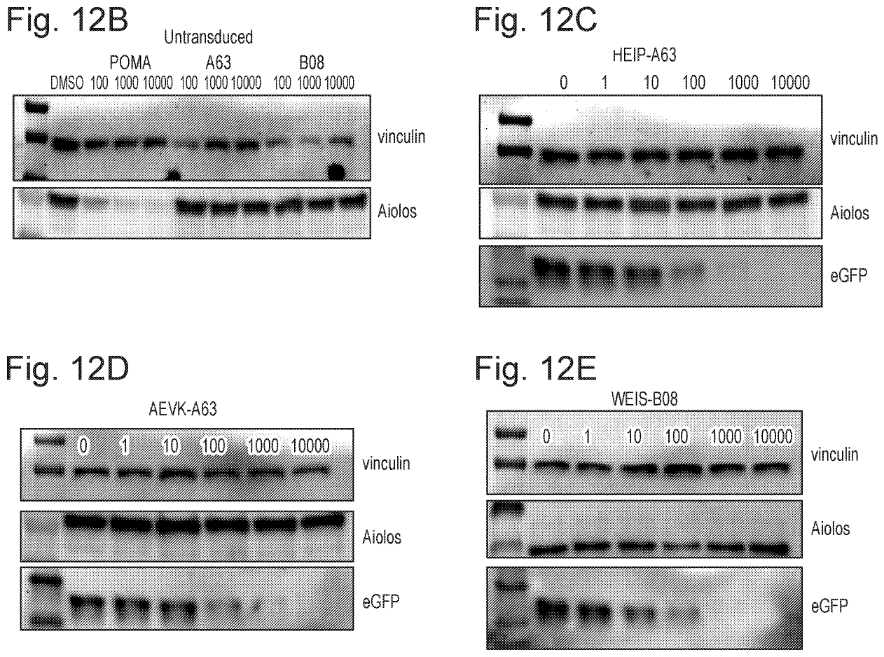

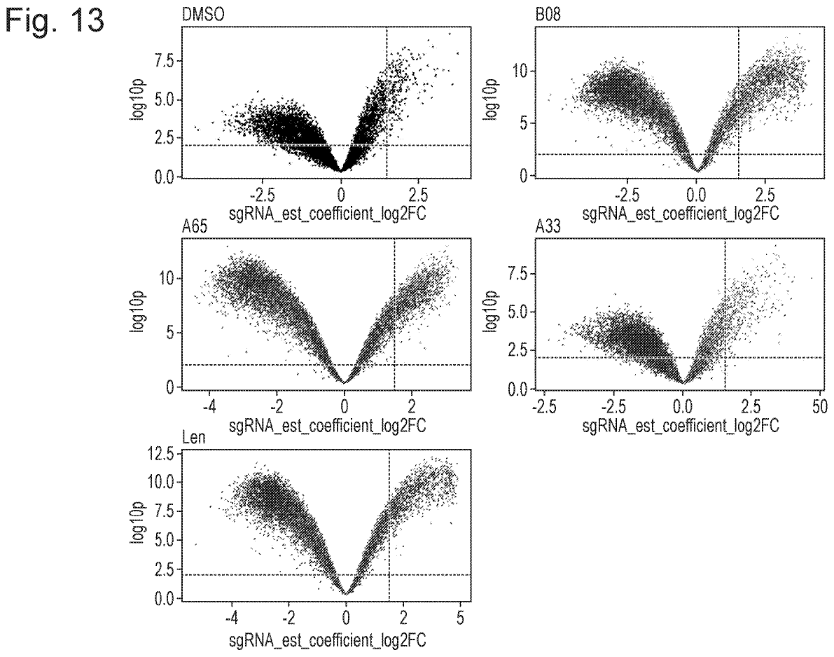

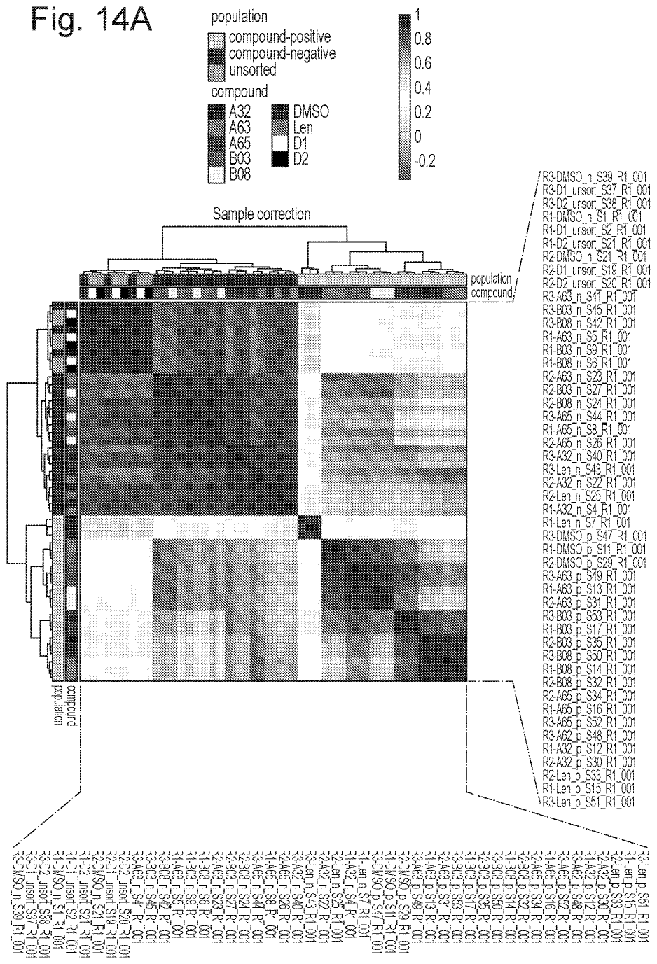

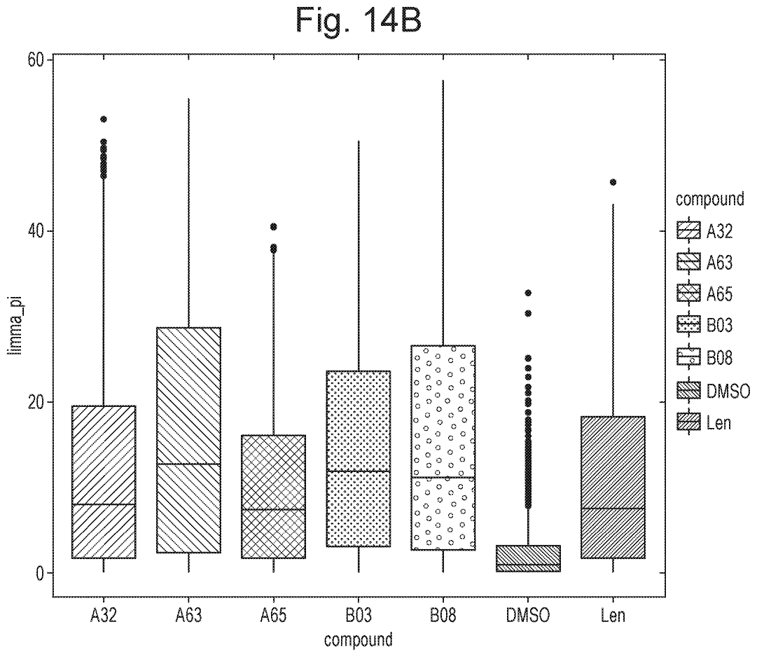

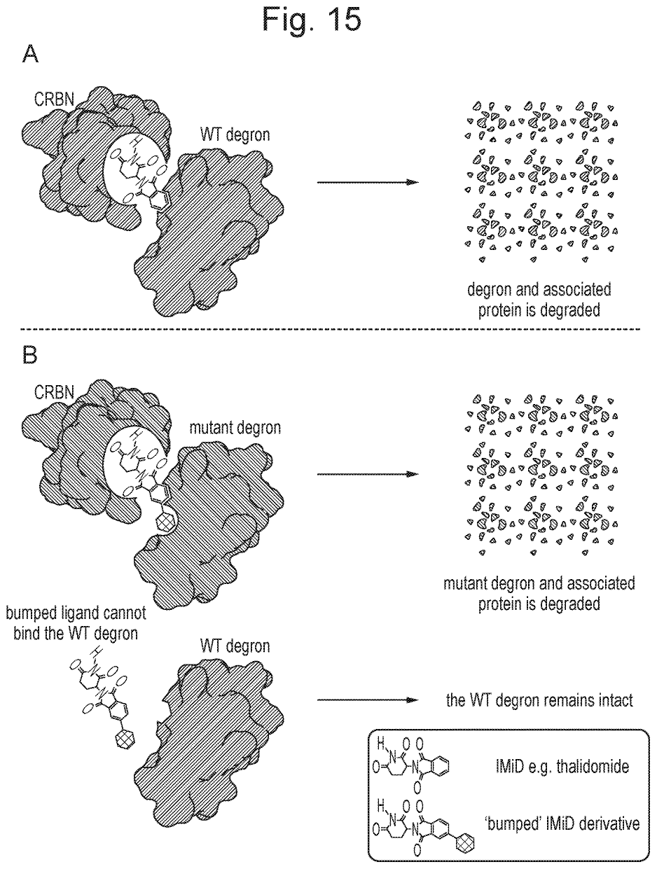

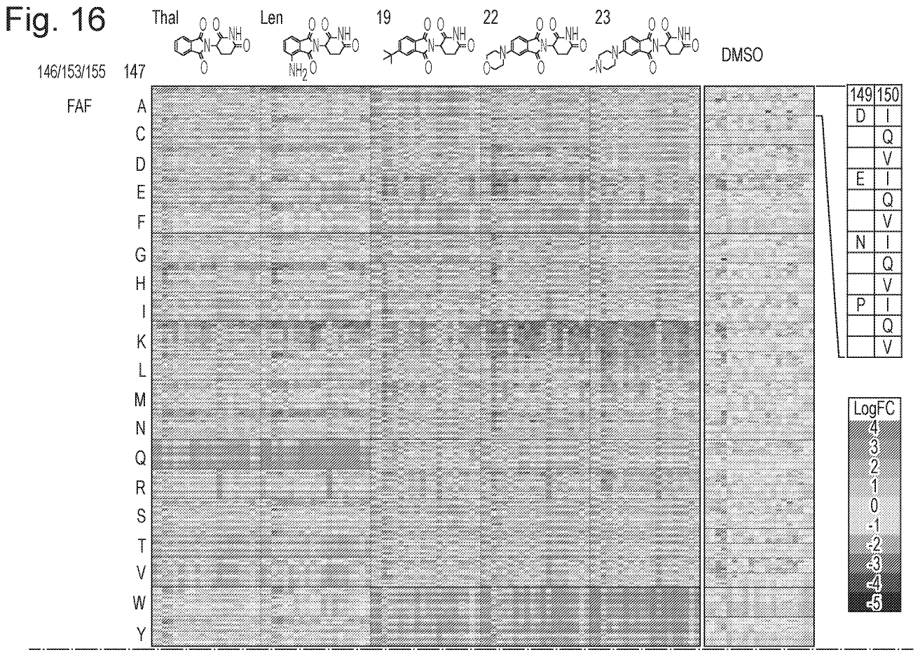

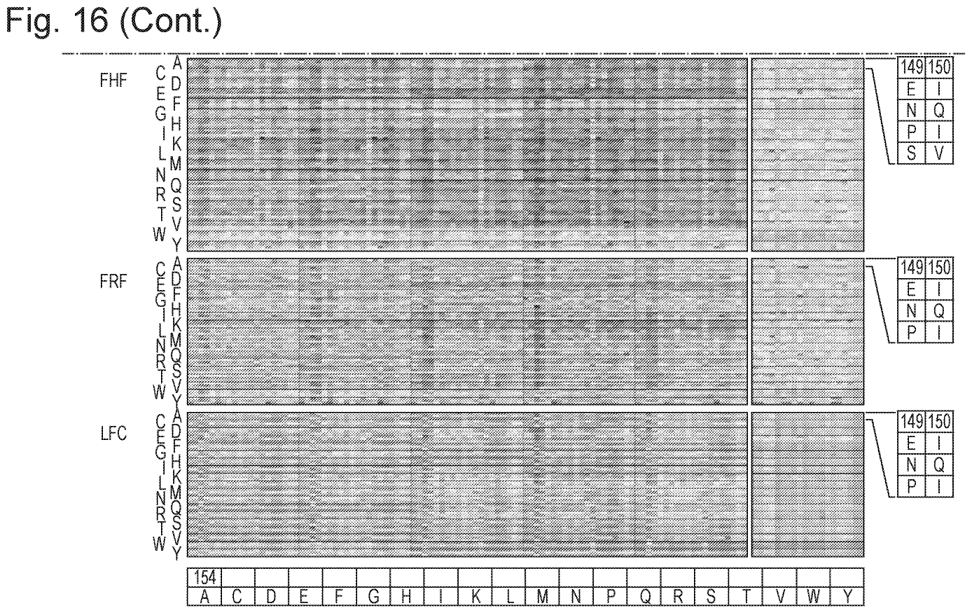

GOLD's inbuilt ‘scaffold’ feature was used to bias the docking results such that the half of the IMID structure deepest within the CRBN binding pocket—the piperidine-2,6-dione six membered ring—remained in the same position and conformation as in the crystal structure; meanwhile the phthalimide bicyclic ring protruding out of CRBN was allowed to be flexible, along with any R groups. For all amino acids (where possible) at 147, the side chain was also allowed to be flexible. Final poses were then assessed using the scoring function from Autodock4 to return a predicted binding energy. The results of this screen (FIG. 7A-B) suggested that an IMID analogue with a benzylamino group on the phthalimide 4 position might possess strong affinity for a ZF with a tryptophan at position 147. The docking pose produced suggested a pi-pi stacking interaction between the phenyl ring of the IMID and the side chain of thetryptophan (FIG. 8C). Starting from this hit, we decided to carry out a second in silico screen on a set of analogues of this IMID (FIG. 8), whereupon some small modifications were investiagted as potential improvements to the original design.

Alongside considering this potential benzyl-tryptophan interaction, a more traditional bump-and-hole approach was considered when choosing which ligands to synthesise in the laboratory. Further inspection of 6HOF led us to the conclusion that position 5 was most suitable for a typical ‘bump’ group to pair with a mutation at position 147.

A library of 36 IMID analogues was synthesised (FIG. 3A), with a variety of molecules representing both the 4 position aminobenzyl modification, and the 5 position ‘classic bump’ modification, as well as several similar analogues.

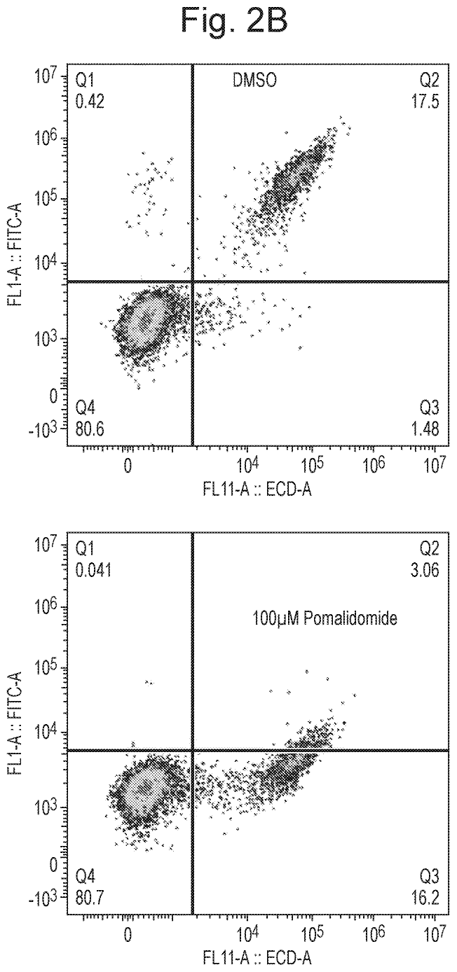

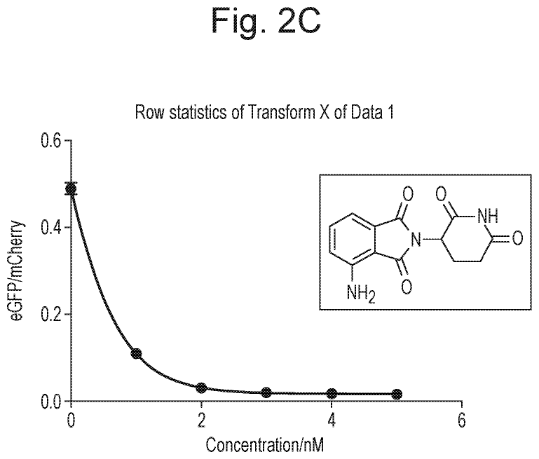

To test these compounds, we used a flow cytometry assay (FIG. 2) employing an Jurkat cells—an immortalised human blood cell line—that had been stably transduced with a plasmid using a lentiviral vector. This plasmid contained a gene for a 60 amino acid ZF region derived from IKZF3 fused to eGFP, separated from mCherry by an internal ribosomal entry site (IRES). This meant that these cells would express ZF-GFP and mCherry in consistent relative amounts, so under normal conditions the ratio of GFP:mCherry would remain the same regardless of overall expression levels. However, upon incubation with an IMID, ZF-GFP would be selectively degraded, altering this ratio and thus providing an easily measurable proxy for level of ZF degradation.

We carried out three biological replicates of this assay for each IMID analogue with a 0.1% DMSO control and six concentrations of IMID (1 nM, 10 nM, 100 nM, 1 μM, 10 μM and 100 μM) for each compound. This yielded a degradation curve for each compound, from which a pDC₅₀ value could be derived, indicating how potent a degrader each of our compound were relative to thalidomide, lenalidomide and pomalidomide.

As expected, most of the IMID analogues with bulky groups at the 5-position were less potent degraders relative to thalidomide. The 4 position aminobenzyl analogues were, however, generally very potent, with several possessing similar potency to pomalidomide.

Other trends noted included the general poor degradation ability of halogenated IMIDs, regardless of whether halogenation was at the 4- or 5-position; the increase of degradation ability afforded by adding a methyl group to the 4-position of thalidomide; and the general trend that IMIDs with aniline substituents possessed greater degradation ability than their phenol substituent counterparts.

To probe the potential for orthogonality in a bump-and-hole context, the same assay was carried out again on all compounds, but this time with a Q147A mutated ZF. An alanine at position 147 was chosen as the benchmark for bump-and-hole as it is more accommodating than the glutamine seen in the WT, but does not suffer from flexibility issues as can arise when mutating to a glycine.

The pDC₅₀ values derived from this screen were used along with the pDC₅₀ values from the WT screen to assign an orthogonality index value to every compound according the following equation:

$$Orthogonality index = 10 \hat{} ( pDC 50 ( Q 147 A ) / pDC 50 ( WT ) )$$

Therefore, any molecule with a value >10 is a more potent degrader of the Q147A mutant ZF than the WT ZF, and is a potential candidate for further screening for bump-and-hole activity (FIG. 3C). After dismissal of any compounds with a Q147A Dₘₐₓ value >=0.4, only three compounds possessed an orthogonality index value >10: A63, A75, and B08. We decided not to pursue A75 (5-hydroxythalidomide) further as this is a degrader of SALL4, another neosubstrate of IMIDs currently in use in medicine. A63 (5-tert-butyl-thalidomide) and B08 (5-(4-methylpiperazin-1-yl)-thalidomide) both possess bulky groups at the 5-position, so were chosen as candidates to take forward.

For either of these candidate molecules to be useful in an orthogonal IMID/ZF degron system, the selectivity of mutant degron over wild-type degron would need to be increased. We therefore decided to screen these candidate IMID analogues against a library of ZF analogues containing multiple mutations to uncover those with enhanced degron characteristics.

Further inspection of the crystal structure 6HOF suggested that, along with Q147, other important residues to consider included 146, 149, 150, 153, 154, 155 and 167. Residues 149, 150, 153 and 167, although not in direct contact with pomalidomide in this structure, are in contact with CRBN; therefore, appropriate mutations could enhance general CRBN/ZF affinity regardless of effects of the IMID molecule itself. Residues 146, 153 and 155 interact in a cooperative manner to make up the hydrophobic core region of the ZF. Alongside the F146/A153/F155 (FAF) motif observed in the IKZF1/IKZF3 ZF, other motifs appear quite frequently in endogenous ZF motifs, including FHF, FRF and LFC. Mutation from A153 to H or R is notable as this perturbs the angle between CRBN and ZF in their binding position. However, in the LFC motif, despite the mutation from A to F at 153, the F side chain in fact occupies the internal region occupied by F155 in IKZF1/IKZF3, rather than clashing with CRBN. Residue 154—a serine in IKZF1/IKZF3—does not interact with either the IMID or CRBN directly, but is proximal to residue 147, so any mutation to 154 is likely to affect the conformation of residue 147, and therefore the overall integrity of the CRBN-IMID-ZF ternary complex. While serine is too small to bridge the CRBN-ZF gap in this region, we noticed that a longer cationic side chain—such as lysine or arginine—may well be long enough to interact with E377 in CRBN and form an ionic interaction.

The total number of residues being considered here is 8: 146, 147, 149, 150, 153, 154, 155, and 167. Therefore, the full number of mutants possible is 20\hat{ }=25,600,000,000, whereas a reasonable genetic library in this instance is around 8,000. We decided to prioritise residues 147 and 154 most heavily to most exhaustively explore all possible direct and indirect interactions with the IMID molecule. We did, however, decide to exclude proline from position 147, as the backbone angles for Q147 in the crystal structure suggested it would not be a good fit.

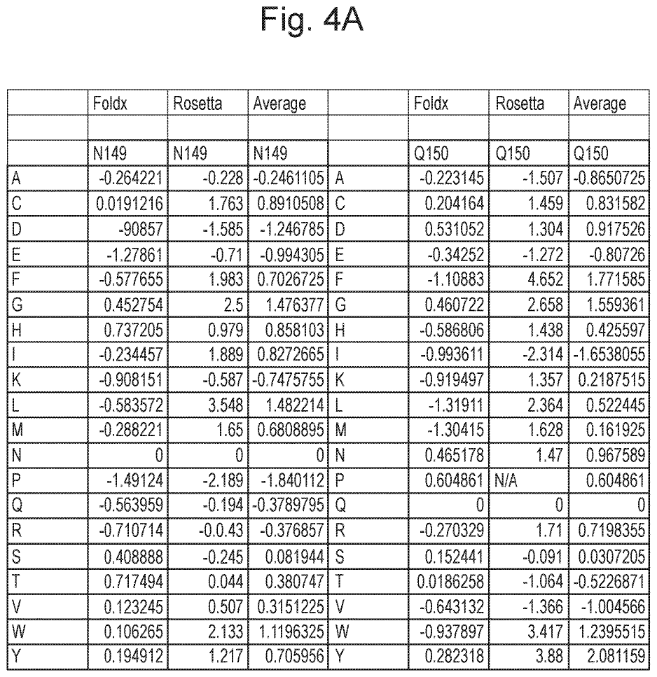

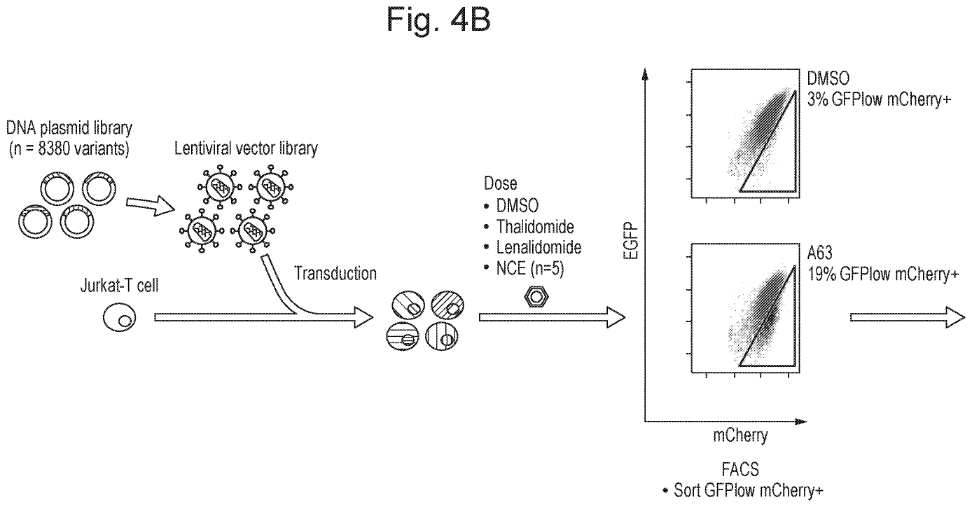

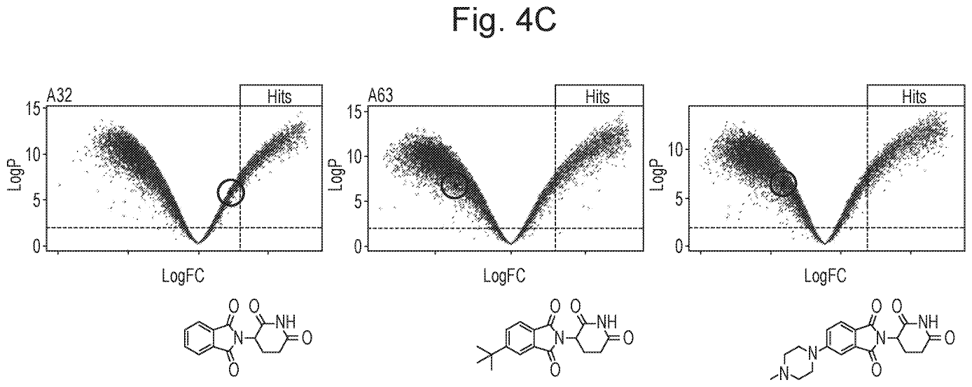

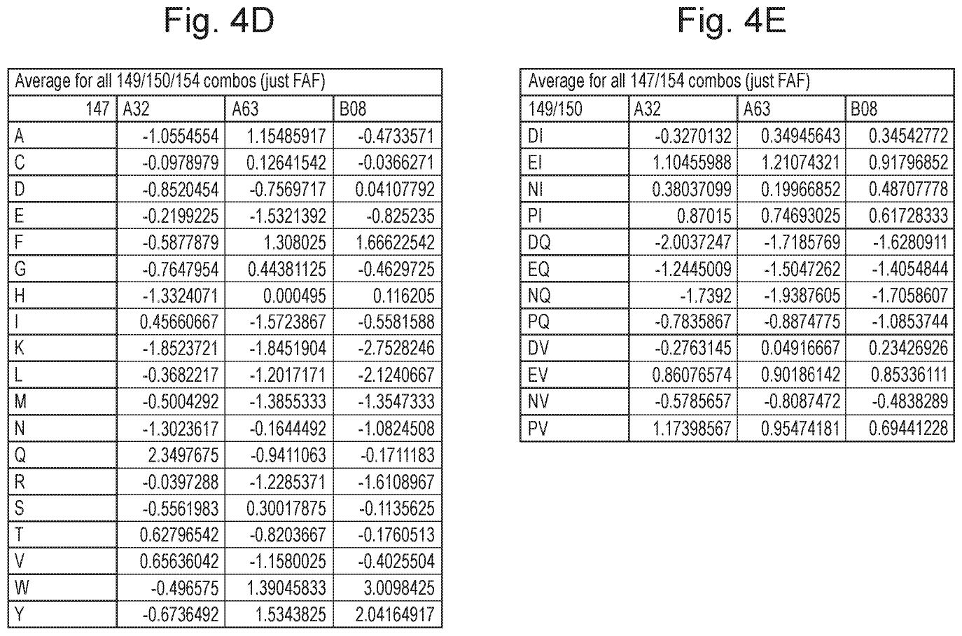

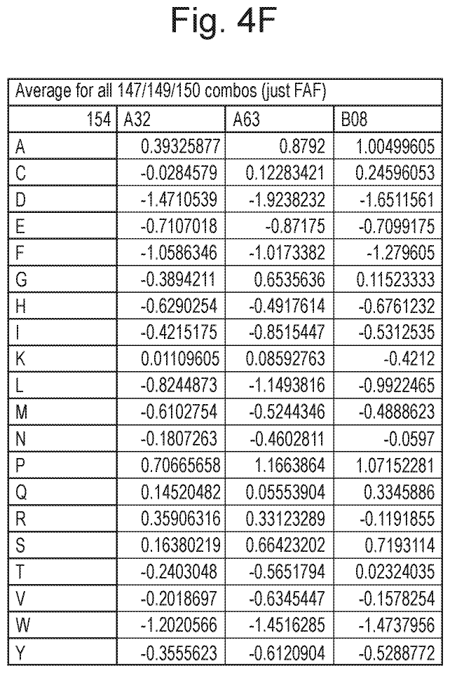

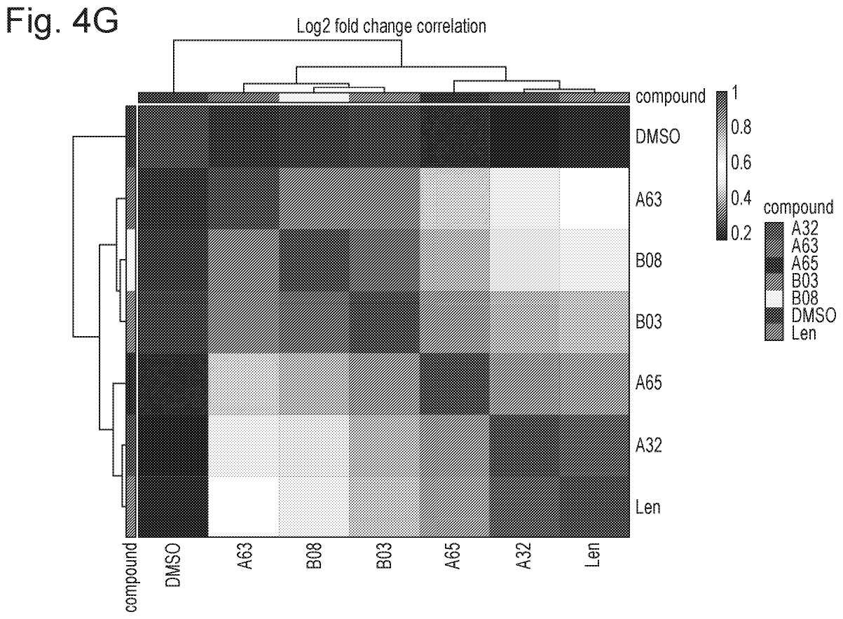

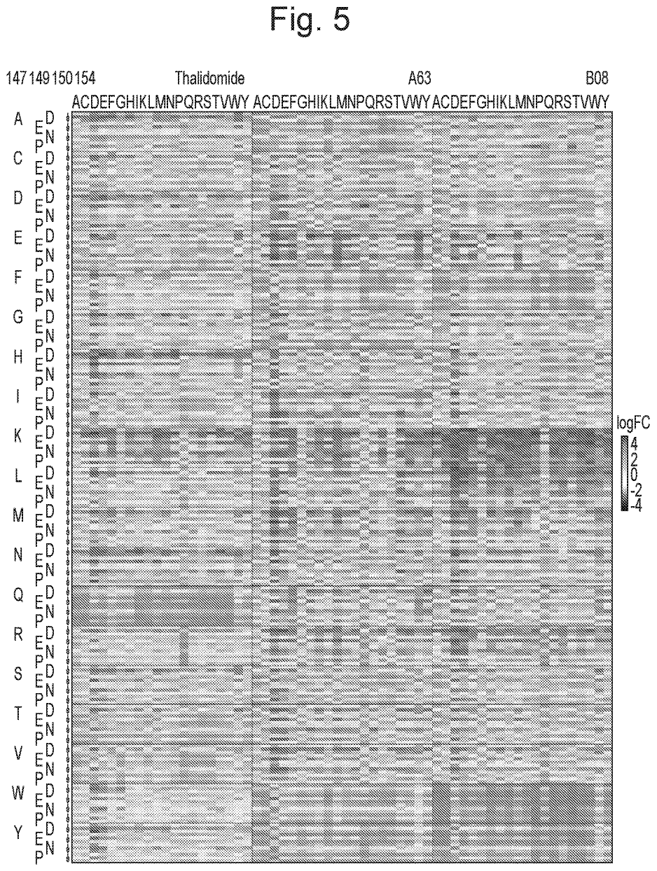

We then used both Foldx and Rosetta to assess which residues would be most enhancing towards CRBN-ZF affinity at positions 149, 150, 153 and 167 (FIG. 4A). Both Foldx's PositionScan and Rosetta's PositionMutation functionalities suggested favourable energies relative to the WT residues at positions 149 and 150 but not 153 or 167. Whilst the different softwares' results did not match up entirely, they found consensus in residues D, E and P for position 149, and I and V for position 150. These residues were also seen to appear relatively commonly in ZFs from other endogenous neosubstrates, building our confidence in these choices.

Therefore, the core of our library was built around all possible combinations of mutations for 147 (all except proline=19), 149 (D, E, N and P=4), 150 (I, Q and V=3) and 154 (all 20), totalling 4,560. In addition to this ‘core’ library we decided to explore some other 146/153/155 motifs as well as the WT ‘FAF’ motif. Therefore, a further 3,420 mutants were added, made up of: 147 (all except proline=19); 154 (all 20); three combinations of 149 and 150: E149/I150, P149/I150 and N149/Q150 (same as WT); and three combinations of 146/153/155: F146/H153/F155, F146/R153/F155, and L146/F153/C155. This added a further 3,420 combinations. Further to the three 149/150 combinations specified, another 149/150 combination was added for the FHF 146/153/155 motif subset: S149 and V150, adding a further 380 combinations. The total is now 8,360. Another 19 combinations were then added: all 20 except proline at 147 with an L167K mutation and all others same as WT. Finally, an extra single G152W (all other positions same as WT) was added as a negative control. The final total library size was therefore 8,380 mutants.

To carry out the library screen, we used the same plasmid construct as in our earlier flow cytometry assays, but pooling all 8,380 mutants together into one Jurkat population, ensuring a copy number of one plasmid per cell (FIG. 4B). After stable transduction of this Jurkat library population, cultures were incubated overnight with an appropriate concentration of IMID to ensure that around 10-15% of the population would then exhibit significant ZF-GFP degradation. These GFP-negative cells were then separated from the remaining GFP-positive population using fluorescence activated cell sorting (FACS). These two populations were then sequenced and the relative abundance of every mutant in both populations compared to provide a log FC value for each mutant sequence. This value indicated how frequently a sequence is observed in the GFP-negative population relative to the GFP-positive population, and therefore how effective a degron it is for that particular IMID compound.

Volcano plots for the thalidomide, A63 and B08 library screens (FIG. 4C) show generally high log P values, confirming the validity of the data. Furthermore, the WT sequence appears in the expected regions for all three of these compounds: close to the ‘hits’ region for thalidomide and far away from the ‘hits’ region for both A63 and B08. This shows firstly that thalidomide pairs fairly well with WT (as would be expected) but that there are a large number of sequences it performs better with. Secondly this shows that, for both A63 and B08, a large proportion of the library is made up of sequences that are more effectively degraded compared to WT.

In general, the mutants containing FRF, FHF and LFC at positions 146/153/155 scored far less well than equivalent mutants with the WT FAF sequence, indicating that the F146/A153/F155 sequence is already well-suited to this degron.

Having assessed the broad trends from this library screen, the sequences with the highest log FC values for A63 and B08 were selected and transduced into separate Jurkat cell lines. The top five performing sequences were selected for A63 (in order—HEIP, FEVP, WEIK, FDIP and AEVK) and three of the top five performing sequences were selected for B08 (WEIS, WNIQ and WDIA). Single mutants at position 147 (ANQS and WNQS) were also expressed to compare against the ‘best’ mutants. Two more mutants were expressed to explore the effects of enhancing mutations at positions 149 and 150 both in and out of a bump-and-hole context: QPIS (for comparison against WT when treated with thalidomide) and APIS (for comparison against ANQS when treated with A63).

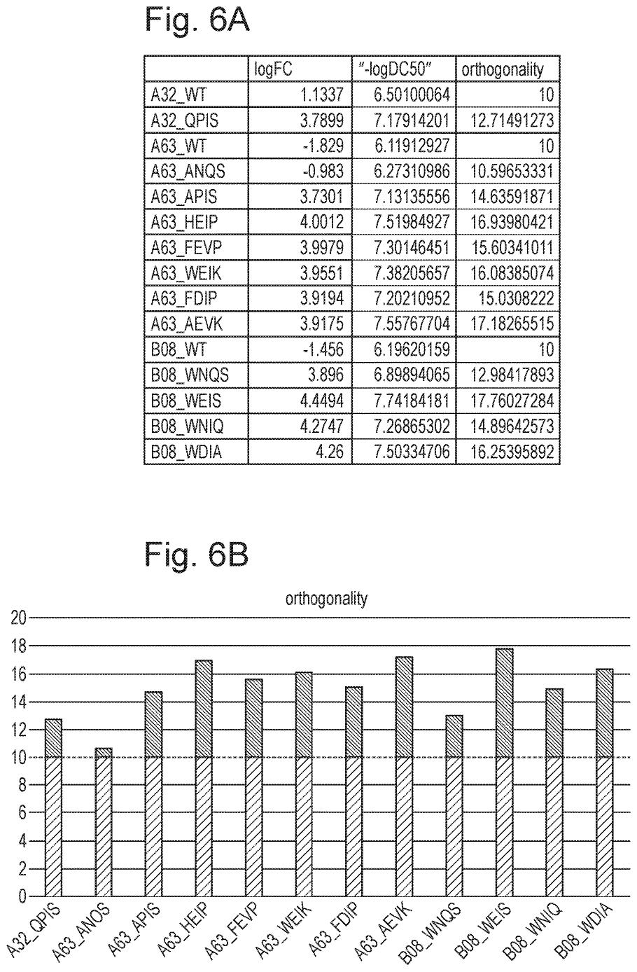

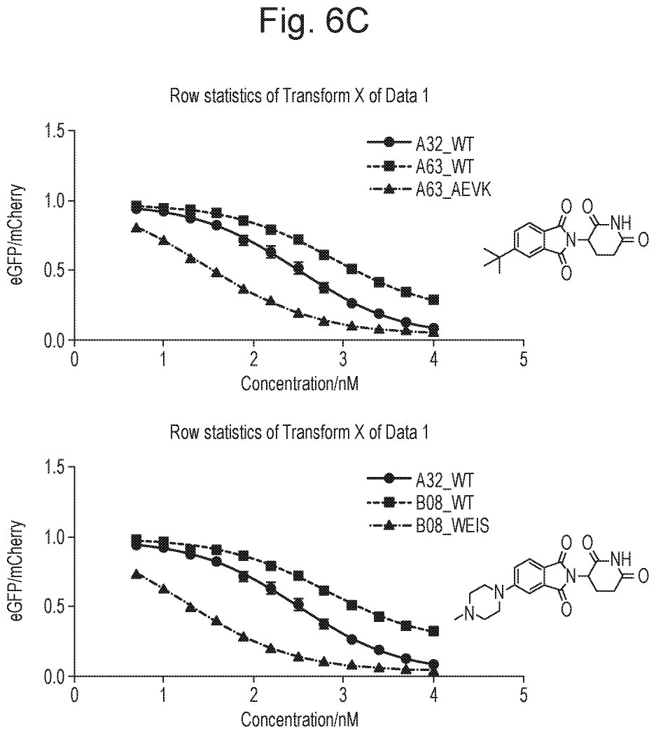

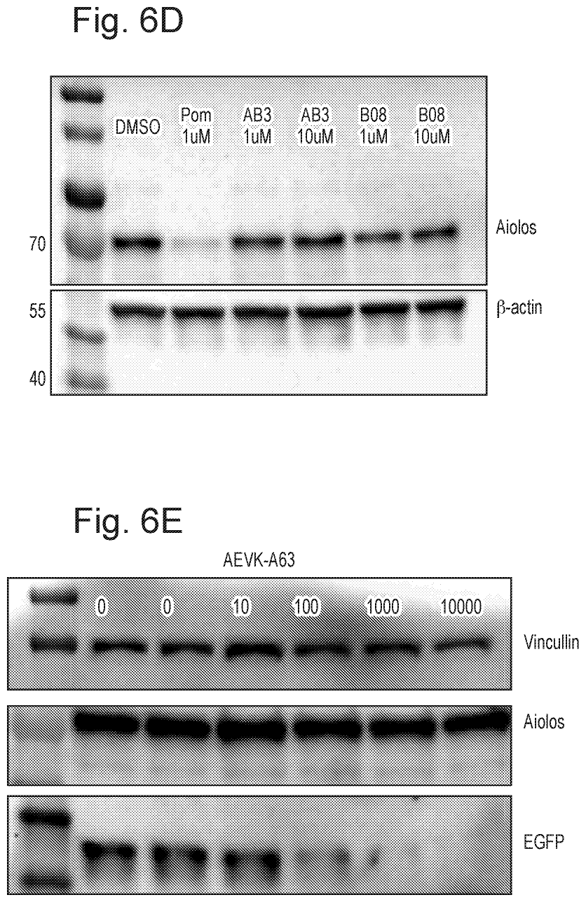

Again, pDC₅₀ values were obtained from degradation curves (this time a concentration curve of 5 nM-10 μM comprising 12 concentrations) and used to calculate orthogonality index values for each IMID-mutant ZF pair (FIG. 6A). These data indicate that best performing mutants are AEVK and WEIS for A63 and B08 respectively, each with around 30× selectivity compared to the WT ZF.

However, while both A63 and B08 exhibit some degradation ability against the WT ZF sequence in the context of this flow cytometry assay, Western blot analysis confirms that neither A63 nor B08 possess ability to degrade endogenous IKZF3, even at 10 uM.

### Example 2

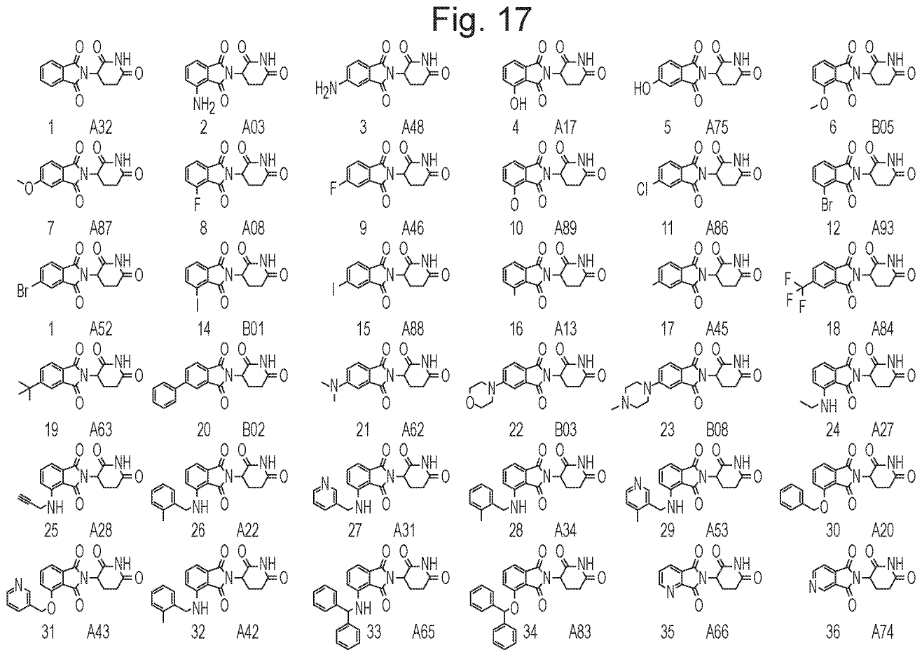

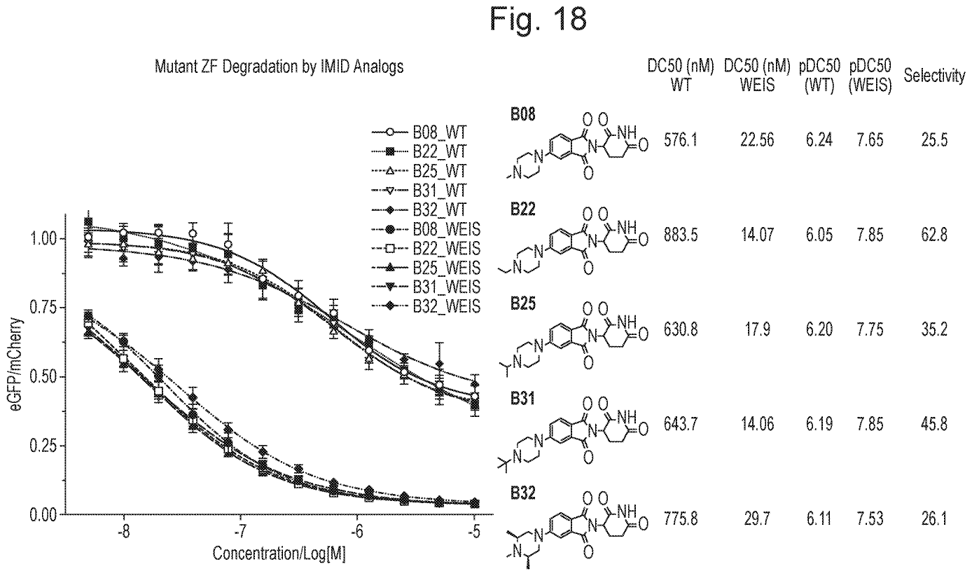

Four additional compounds, B22, B25, B31 and B32 (see FIG. 18) were synthesized in accordance with the general methods disclosed herein. Compounds could be synthesised by analogy with 2-(2,6-dioxopiperidin-3-yl)-5-(4-methylpiperazin-1-yl)isoindoline-1,3-dione above using suitably substituted piperazine (1-ethylpiperazine, 1-isopropylpiperazine, 1-tertbutylpiperazine, 1, 2, 6-trimethylpiperazine) as reagent.

The compounds were assessed in GFP degradation flow cytometry assays as described above. pDC values against wild type IKZF3 and WEIS mutant (Q147W N149E Q150I) were established demonstrating the impressive degradation selectivity of the tested compounds.

### Example 3

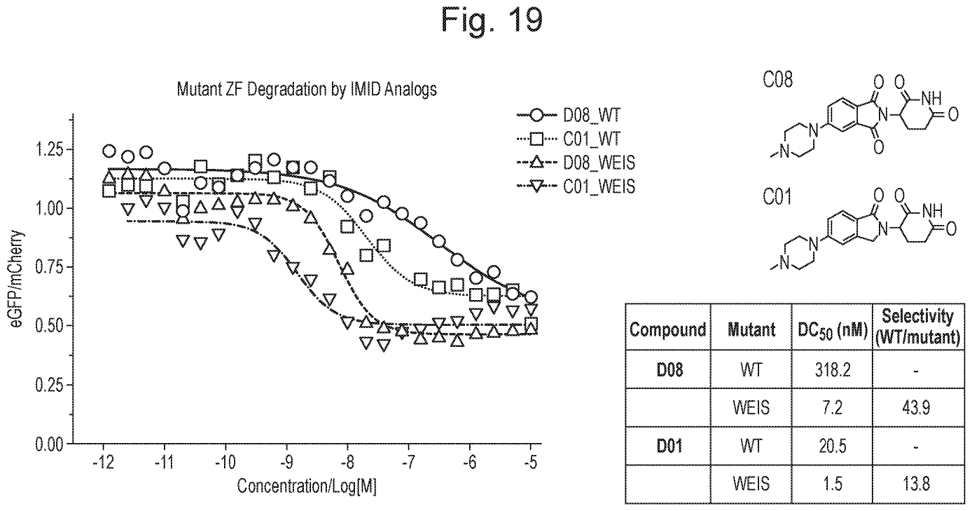

An additional compound, C01 (see FIG. 19) was synthesized in accordance with the general methods disclosed herein. C01 could be synthesised by analogy with 2-(2,6-dioxopiperidin-3-yl)-5-(4-methylpiperazin-1-yl)isoindoline-1,3-dione above using 3-(5-fluoro-1-oxoisoindolin-2-yl)piperidine-2,6-dione as reagent.

The compounds were assessed in GFP degradation assays as described above. (Data shown in FIG. 19 was obtained using a plate reader leading to a degree of background autofluorescence). The data in FIG. 19 indicates the inflection point—at which range of concentrations the degradation occurs—for each compound. DC₅₀ and selectivity values (following subtraction of background autofluorescence) were acquired. Compound C01 degrades both wild type IKZF3 and WEIS mutant (Q147W N149E Q150I).

1. A system, comprising:
i) a polypeptide comprising a variant of the amino acid sequence of SEQ ID NO: 1, wherein the variant comprises a modification at the position corresponding to Q147 of SEQ ID NO: 2;
and
ii) a compound of Formula (A) 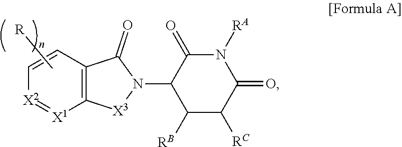
wherein
X¹ is C—R and X² is C—R or N; or X¹ is C—R or N and X² is C—R;
X³ is CHR^{D} or C═O;
each R, which may be the same or different, is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
wherein each alkyl, alkenyl and alkynyl group is independently unsubstituted or is substituted with 1, 2 or 3 substituents independently selected from C₆₋₁₀ aryl, 5- to 10-membered heteroaryl, 5- to 10-membered heterocyclyl, C₅₋₁₀ carbocyclyl, OR²⁰, N(R²⁰)₂ and halogen;
and wherein each aryl, heteroaryl, heterocyclyl and carbocyclyl group is independently unsubstituted or is substituted with 1, 2 or 3 substituents independently selected from R²⁰, OR²⁰, N(R²⁰)₂ and halogen;
each R¹⁰, which may be the same or different, is independently H or C₁₋₄ alkyl which is unsubstituted or substituted with 1, 2 or 3 substituents independently selected from OR²⁰, N(R²⁰)₂ and halogen;
n is an integer from 0 to 2;
R^{A} is H or C₁₋₄ alkyl which is unsubstituted or is substituted with 1, 2 or 3 substituents independently selected from OR²⁰, N(R²⁰)₂ and halogen;
R^{B} is H or C₁₋₄ alkyl which is unsubstituted or is substituted with 1, 2 or 3 substituents independently selected from OR²⁰, N(R²⁰)₂ and halogen;
R^{C} is H or C₁0.4 alkyl which is unsubstituted or is substituted with 1, 2 or 3 substituents independently selected from OR²⁰, N(R²⁰)₂ and halogen;
R^{D} is H or C₁0.4 alkyl which is unsubstituted or is substituted with 1, 2 or 3 substituents independently selected from OR²⁰, N(R²⁰)₂ and halogen; and
each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl.

2. A system according to claim 1, wherein the variant comprises a modification at one or more of the positions corresponding to N149, Q150 and S154 of SEQ ID NO: 2.

3. A system according to claim 1, wherein the variant comprises a modification at the positions corresponding to Q147, N149 and Q150 of SEQ ID NO: 2.

4. A system according to any one of claims 1 to 3, wherein the variant comprises one or more of the modifications:
i) Q147 Å/C/F/G/H/N/S/W/Y relative to SEQ ID NO: 2, preferably Q147 Å/F/H/W; and/or
ii) N149D/E/N/P relative to SEQ ID NO: 2; preferably N149D/E; and/or
iii) Q150I/Q/V relative to SEQ ID NO: 2; preferably Q1501/V; and/or
iv) S154 Å/C/E/F/G/H/I/K/L/M/N/P/Q/R/S/T/V/Y relative to SEQ ID NO: 2; preferably S154K/P/Q/A; and/or
v) A153F/H/R relative to SEQ ID NO: 2; preferably A153F; and/or
vi) F146L relative to SEQ ID NO: 2; and/or
vii) F155C relative to SEQ ID NO: 2.

5. A system according to any one of claims 1 to 3, wherein the variant comprises:
Q147H/N149E/Q150I/S154P relative to SEQ ID NO: 2; or
Q147F/N149E/Q150V/S154P; or
Q147W/N149E/Q1501/S154K; or
Q147F/N149D/Q1501/S154P; or
Q147A/N149E/Q150V/S154K; or
Q147W/N149E/Q1501; or
Q147W/Q150I/S154Q; or
Q147W/N149D/Q150I/S154A.

6. A system according to any one of claims 1 to 5, wherein the polypeptide is attached to a target protein.

7. A system according to any one of claims 1 to 6, wherein the polypeptide is attached to the target protein by genetic fusion and/or via a linker.

8. A system according to any one of claims 1 to 7, wherein the compound of Formula (A) is a compound of Formula (I): 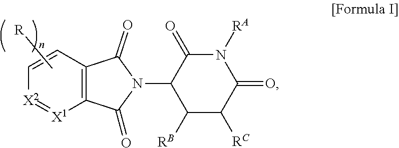
wherein
X¹ is C—R and X² is C—R or N; or X¹ is C—R or N and X² is C—R;
each R, which may be the same or different, is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
wherein each alkyl, alkenyl and alkynyl group is independently unsubstituted or is substituted with 1, 2 or 3 substituents independently selected from C₆₋₁₀ aryl, 5- to 10-membered heteroaryl, 5- to 10-membered heterocyclyl, C₅₋₁₀ carbocyclyl, OR²⁰, N(R²⁰)₂ and halogen;
and wherein each aryl, heteroaryl, heterocyclyl and carbocyclyl group is independently unsubstituted or is substituted with 1, 2 or 3, preferably 1 or 2 substituents independently selected from R²⁰, OR²⁰, N(R²⁰)₂ and halogen;
each R¹⁰, which may be the same or different, is independently H or C₁₋₄ alkyl which is unsubstituted or substituted with 1, 2 or 3 substituents independently selected from OR²⁰, N(R²⁰)₂ and halogen;
n is an integer from 0 to 2;
R^{A} is H or C₁₋₄ alkyl which is unsubstituted or is substituted with 1, 2 or 3 substituents independently selected from OR²⁰, N(R²⁰)₂ and halogen;
R^{B} is H or C₁₋₄ alkyl which is unsubstituted or is substituted with 1, 2 or 3 substituents independently selected from OR²⁰, N(R²⁰)₂ and halogen;
R^{C} is H or C₁₋₄ alkyl which is unsubstituted or is substituted with 1, 2 or 3 substituents independently selected from OR²⁰, N(R²⁰)₂ and halogen; and
each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl; preferably each R²⁰ is independently H or methyl.

9. A system according to claim 8, wherein in formula (I):
n is 0; and/or
R^{A} is H or methyl, preferably H; and/or
R^{B} is H or methyl, preferably H; and/or
R^{C} is H or methyl, preferably H; and/or
X¹ is C—R and X² is C—R.

10. A system according to claim 8 or claim 9, wherein the compound of formula (I) is a compound of formula (Ta):
wherein 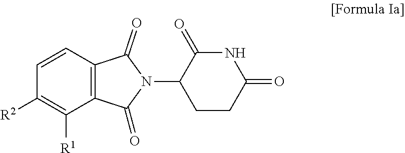
R¹ is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
wherein each alkyl, alkenyl and alkynyl group is independently unsubstituted or is substituted with 1, 2 or 3 substituents independently selected from C₆₋₁₀ aryl, 5- to 10-membered heteroaryl, 5- to 10-membered heterocyclyl, C₅₋₁₀ carbocyclyl, OR²⁰, N(R²⁰)₂ and halogen;
and wherein each aryl, heteroaryl, heterocyclyl and carbocyclyl group is independently unsubstituted or is substituted with 1, 2 or 3, preferably 1 or 2 substituents independently selected from R²⁰, OR²⁰, N(R²⁰)₂ and halogen;
R² is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
wherein each alkyl, alkenyl and alkynyl group is independently unsubstituted or is substituted with 1, 2 or 3 substituents independently selected from C₆₋₁₀ aryl, 5- to 10-membered heteroaryl, 5- to 10-membered heterocyclyl, C₅₋₁₀ carbocyclyl, OR²⁰, N(R²⁰)₂ and halogen;
and wherein each aryl, heteroaryl, heterocyclyl and carbocyclyl group is independently unsubstituted or is substituted with 1, 2 or 3 substituents independently selected from R²⁰, OR²⁰, N(R²⁰)₂ and halogen;
each R¹⁰, which may be the same or different, is independently H or methyl; and
each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl; preferably each R²⁰ is independently H or methyl.

11. A system according to any one of claims 1 to 9, wherein R¹ is selected from hydrogen, N(R¹⁰)₂, OR¹⁰, halogen, C₁₋₄ alkyl, C₂₋₄ alkynyl, O—C₁₋₄ alkyl, O—C₂₋₄ alkynyl, NR¹⁰—C₁₋₄ alkyl, NR¹⁰—C₂₋₄ alkynyl, C₆ aryl, and 5- to 6-membered heterocyclyl, and 5- to 6-membered heteroaryl;
wherein each alkyl and alkynyl group is independently unsubstituted or is substituted with 1, 2 or 3 substituents independently selected from C₆ aryl, 5- to 6-membered heteroaryl, and halogen;
and wherein each aryl, heteroaryl, heterocyclyl and carbocyclyl group is independently unsubstituted or is substituted with 1, 2, or 3 R²⁰ groups, preferably one R²⁰ group.

12. A system according to any one of claims 1 to 9, wherein R² is selected from hydrogen, N(R¹⁰)₂, OR¹⁰, halogen, C₁₋₄ alkyl, C₂₋₄ alkynyl, O—C₁₋₄ alkyl, O—C₂₋₄ alkynyl, NR¹⁰—C₁₋₄ alkyl, NR¹⁰—C₂₋₄ alkynyl, C₆ aryl, and 5- to 6-membered heterocyclyl, and 5- to 6-membered heteroaryl;
wherein each alkyl and alkynyl group is independently unsubstituted or is substituted with 1, 2 or 3 substituents independently selected from C₆ aryl, 5- to 6-membered heteroaryl, and halogen;
and wherein each aryl, heteroaryl, and heterocyclyl group is independently unsubstituted or is substituted with 1, 2, or 3 R²⁰ groups, preferably one R²⁰ group.

13. A system according to any one of claims 1 to 9, wherein:
i) R¹ is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered heterocyclyl;
ii) R² is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered heterocyclyl;
wherein each alkyl and alkynyl group is independently unsubstituted or is substituted with 1 or 2 substituents independently selected from C₆ aryl, 5- to 6-membered heteroaryl or with 1-3 halogen substituents;
and wherein each aryl, heteroaryl, and heterocyclyl group is independently unsubstituted or is substituted with 1, 2, or 3 R²⁰ groups, preferably one R²⁰ group.

14. A system according to any one of claims 1 to 9, wherein:
i) R¹ is selected from H, NH₂, OH, OMe, halogen, methyl, NH—C₁₋₃ alkyl, and unsubstituted NH—C₃ alkynyl;
wherein each C₁₋₃ alkyl group is independently unsubstituted or is substituted with 1 or 2 phenyl or pyridinyl groups; wherein said phenyl or pyridinyl groups are each independently unsubstituted or substituted by methyl; and
ii) R² is selected from H, N(R¹⁰)₂, OH, OMe, halogen, unsubstituted C₁₋₄ alkyl, CF₃, phenyl, piperazinyl, and morpholinyl;
wherein said phenyl, piperazinyl, and morpholinyl groups are independently unsubstituted or substituted by C₁₋₆ alkyl, preferably C₁₋₄ alkyl, more preferably methyl.

15. A system according to claim 10, wherein R¹ and R² are defined as any one of the combinations:

| Compound | | |
|---|---|---|
| number | R¹ | R² |
| 1 | 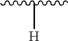 [SMILES: *[H]] | 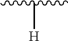 [SMILES: *[H]] |
| 2 | 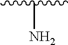 [SMILES: *N] | 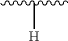 [SMILES: *[H]] |
| 3 | 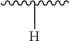 [SMILES: *[H]] | 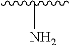 [SMILES: *N] |
| 4 | 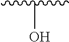 [SMILES: *O] | 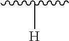 [SMILES: *[H]] |
| 5 | 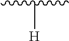 [SMILES: *[H]] | 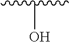 [SMILES: *O] |
| 6 | 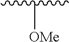 [SMILES: *OC] | 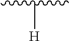 [SMILES: *[H]] |
| 7 | 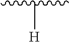 [SMILES: *[H]] | 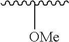 [SMILES: *OC] |
| 8 | 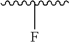 [SMILES: *F] | 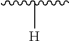 [SMILES: *[H]] |
| 9 | 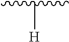 [SMILES: *[H]] | 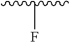 [SMILES: *F] |
| 10 | 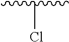 [SMILES: *Cl] | 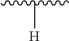 [SMILES: *[H]] |
| 11 | 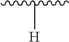 [SMILES: *[H]] | 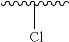 [SMILES: *Cl] |
| 12 | 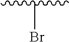 [SMILES: *Br] | 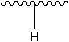 [SMILES: *[H]] |
| 13 | 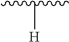 [SMILES: *[H]] | 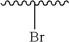 [SMILES: *Br] |
| 14 | 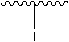 [SMILES: *I] | 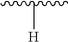 [SMILES: *[H]] |
| 15 | 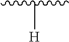 [SMILES: *[H]] | 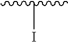 [SMILES: *I] |
| 16 | 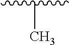 | 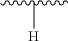 [SMILES: *[H]] |
| 17 | 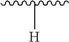 [SMILES: *[H]] | 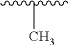 |
| 18 | 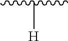 [SMILES: *[H]] | 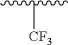 [SMILES: *C(F)(F)F] |
| 19 | 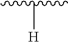 [SMILES: *[H]] | 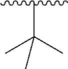 [SMILES: *C(C)(C)C] |
| 20 | 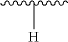 [SMILES: *[H]] | 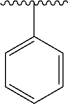 [SMILES: *c1ccccc1] |
| 21 | 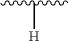 [SMILES: *[H]] | 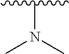 |
| 22 | 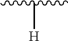 [SMILES: *[H]] | 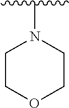 [SMILES: *N1CCOCC1] |
| 23 | 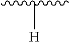 [SMILES: *[H]] | 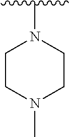 [SMILES: *N1CCN(C)CC1] |
| 24 | 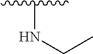 [SMILES: *NCC] | 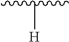 [SMILES: *[H]] |
| 25 | 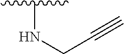 [SMILES: *NCC#C] | 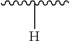 [SMILES: *[H]] |
| 26 | 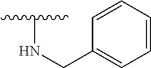 [SMILES: *NCc1ccccc1] | 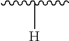 [SMILES: *[H]] |
| 27 | 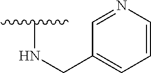 [SMILES: *NCc1cccnc1] | 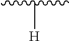 [SMILES: *[H]] |
| 28 | 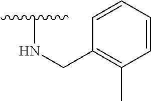 [SMILES: *NCc1ccccc1C] | 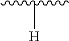 [SMILES: *[H]] |
| 29 | 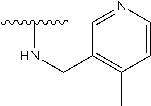 [SMILES: *NCc1cnccc1C] | 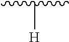 [SMILES: *[H]] |
| 30 | 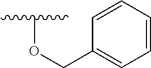 [SMILES: *OCc1ccccc1] | 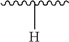 [SMILES: *[H]] |
| 31 | 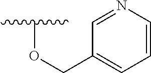 [SMILES: *OCc1cccnc1] | 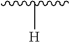 [SMILES: *[H]] |
| 32 | 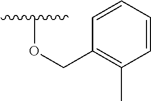 [SMILES: *OCc1ccccc1C] | 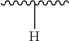 [SMILES: *[H]] |
| 33 | 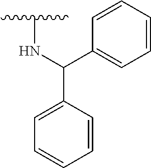 [SMILES: *NC(c1ccccc1)c1ccccc1] | 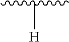 [SMILES: *[H]] |
| 34 | 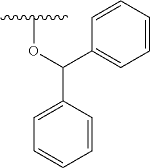 [SMILES: *OC(c1ccccc1)c1ccccc1] | 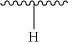 [SMILES: *[H]] |
| 35 | 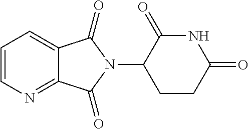 | |
| 36 | 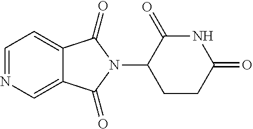 | |
| 37 | 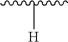 [SMILES: *[H]] | 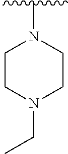 [SMILES: *N1CCN(CC)CC1] |
| 38 | 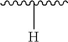 [SMILES: *[H]] | 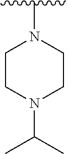 [SMILES: *N1CCN(C(C)C)CC1] |
| 39 | 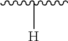 [SMILES: *[H]] | 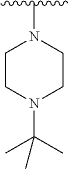 [SMILES: *N1CCN(C(C)(C)C)CC1] |
| 40 | 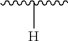 [SMILES: *[H]] | 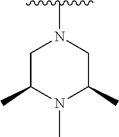 |

16. A system according to any one of claims 1 to 7, wherein the wherein the compound of Formula (A) is a compound of Formula (Id): 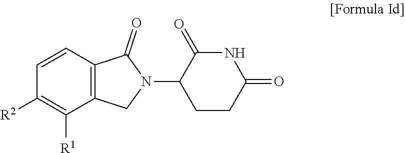
and wherein R¹ and R² are defined as in any one of claims 10 to 15.

17. A polypeptide comprising a variant of the amino acid sequence of SEQ ID NO: 1, wherein the variant comprises (i) a modification at the position corresponding to Q147 of SEQ ID NO: 2; and (ii) a modification at one or more of the positions corresponding to N149, Q150 and S154 of SEQ ID NO: 2.

18. A polypeptide according to claim 17, wherein the variant is as defined according to any one of claims 4 to 7.

19. A system according to any one of claims 1 to 16, or a polypeptide according to claim 17 or claim 18, wherein said polypeptide has at least 70% identity to SEQ ID NO: 2.

20. A system according to any one of claims 1 to 16 or 19, or a polypeptide according to claim 17 or claim 18, wherein said polypeptide is capable of binding a compound of Formula (A).

21. A system according to any one of claims 1 to 16, 19 or 20, or a polypeptide according to claim 17 or claim 18, wherein the binding of said polypeptide to a compound of Formula (A) is stronger than the binding of SEQ ID NO: 2 to said compound of Formula (A).

22. A polynucleotide encoding the polypeptide according to any one of claims 17 to 18.

23. A polynucleotide according to claim 22, wherein the polynucleotide further encodes a protein of interest in-frame relative to the encoded polypeptide.

24. A vector comprising the polynucleotide according to claim 22 or claim 23.

25. A cell expressing the polypeptide according to any one of claims 17 to 18, or comprising the polynucleotide according to claim 22 or 23, or comprising the vector according to claim 24.

26. A compound of Formula (B), 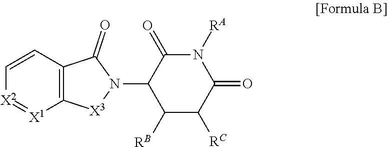
wherein
X¹ is C—R¹ and X² is C—R² or N; or X¹ is C—R¹ or N and X² is C—R²;
X³ is CHR^{D} or C═O;
A:
R¹ is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl; and
R² is selected from N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
or
B:
R¹ is selected from OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl; and
R² is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
wherein each alkyl, alkenyl and alkynyl group is independently unsubstituted or is substituted with 1, 2 or 3 substituents independently selected from C₆₋₁₀ aryl, 5- to 10-membered heteroaryl, 5- to 10-membered heterocyclyl, C₅₋₁₀ carbocyclyl, OR²⁰, N(R²⁰)₂ and halogen;
and wherein each aryl, heteroaryl, heterocyclyl and carbocyclyl group is independently unsubstituted or is substituted with 1, 2 or 3 substituents independently selected from R²⁰, OR²⁰, N(R²⁰)₂ and halogen;
R^{A} is H or C₁₋₄ alkyl which is unsubstituted or is substituted with 1, 2 or 3 substituents independently selected from OR²⁰, N(R²⁰)₂ and halogen;
R^{B} is H or C₁₋₄ alkyl which is unsubstituted or is substituted with 1, 2 or 3 substituents independently selected from OR²⁰, N(R²⁰)₂ and halogen;
R^{C} is H or C₁₋₄ alkyl which is unsubstituted or is substituted with 1, 2 or 3 substituents independently selected from OR²⁰, N(R²⁰)₂ and halogen;
R^{D} is H or C₁₋₄ alkyl which is unsubstituted or is substituted with 1, 2 or 3 substituents independently selected from OR²⁰, N(R²⁰)₂ and halogen; and
each R¹⁰, which may be the same or different, is independently H or C₁₋₄ alkyl which is unsubstituted or substituted with 1, 2 or 3 substituents independently selected from OR²⁰, N(R²⁰)₂ and halogen;
each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl.

27. The compound according to claim 26, wherein the compound of Formula (B) is a compound of Formula (Ib): 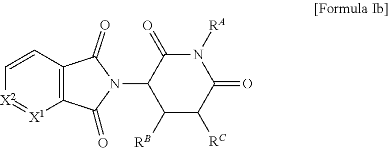
wherein
X¹ is C—R¹ and X² is C—R² or N; or X¹ is C—R¹ or N and X² is C—R²;
A:
R¹ is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl; and
R² is selected from N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
or
B:
R¹ is selected from OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl; and
R² is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
wherein each alkyl, alkenyl and alkynyl group is independently unsubstituted or is substituted with 1, 2 or 3 substituents independently selected from C₆₋₁₀ aryl, 5- to 10-membered heteroaryl, 5- to 10-membered heterocyclyl, C₅₋₁₀ carbocyclyl, OR²⁰, N(R²⁰)₂ and halogen;
and wherein each aryl, heteroaryl, heterocyclyl and carbocyclyl group is independently unsubstituted or is substituted with 1, 2 or 3, preferably 1 or 2 substituents independently selected from R²⁰, OR²⁰, N(R²⁰)₂ and halogen;
R^{A} is H or C₁₋₄ alkyl which is unsubstituted or is substituted with 1, 2 or 3 substituents independently selected from OR²⁰, N(R²⁰)₂ and halogen;
R^{B} is H or C₁₋₄ alkyl which is unsubstituted or is substituted with 1, 2 or 3 substituents independently selected from OR²⁰, N(R²⁰)₂ and halogen;
R^{C} is H or C₁₋₄ alkyl which is unsubstituted or is substituted with 1, 2 or 3 substituents independently selected from OR²⁰, N(R²⁰)₂ and halogen; and
each R¹⁰, which may be the same or different, is independently H or C₁₋₄ alkyl which is unsubstituted or substituted with 1, 2 or 3 substituents independently selected from OR²⁰, N(R²⁰)₂ and halogen;
each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl; preferably each R²⁰ is independently H or methyl.

28. A compound according to claim 26 or claim 27, wherein said compound is a compound of formula (Ia), 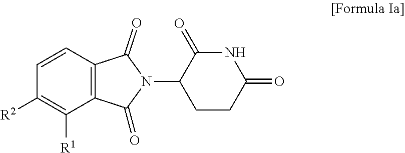
wherein
A:
R¹ is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl; and
R² is selected from N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
or
B:
R¹ is selected from OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl; and
R² is selected from hydrogen, N(R¹⁰)₂, OR¹⁰, halogen, C₁₋₆ alkyl, C₂₋₆ alkenyl, C₂₋₆ alkynyl, O—C₁₋₆ alkyl, O—C₂₋₆ alkenyl, O—C₂₋₆ alkynyl, NR¹⁰—C₁₋₆ alkyl, NR¹⁰—C₂₋₆ alkenyl, NR¹⁰—C₂₋₆ alkynyl, C₆₋₁₀ aryl, 5- to 10-membered heteroaryl, 5- to 10-membered heterocyclyl, and C₅₋₁₀ carbocyclyl;
wherein each alkyl, alkenyl and alkynyl group is independently unsubstituted or is substituted with 1, 2 or 3 substituents independently selected from C₆₋₁₀ aryl, 5- to 10-membered heteroaryl, 5- to 10-membered heterocyclyl, C₅₋₁₀ carbocyclyl, OR²⁰, N(R²⁰)₂ and halogen;
and wherein each aryl, heteroaryl, heterocyclyl and carbocyclyl group is independently unsubstituted or is substituted with 1, 2 or 3, preferably 1 or 2 substituents independently selected from R²⁰, OR²⁰, N(R²⁰)₂ and halogen;
each R¹⁰, which may be the same or different, is independently H or methyl; and
each R²⁰, which may be the same or different, is independently H or C₁₋₆ alkyl which is independently unsubstituted or is substituted with 1, 2 or 3 substituents independently selected from OR³⁰, N(R³⁰)₂ and halogen, wherein R³⁰ is H or methyl; preferably each R²⁰ is independently H or methyl.

29. A compound according to claim 26 or claim 27, wherein:
i) R¹ is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered heterocyclyl;
ii) R² is selected from N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered heterocyclyl;
wherein each alkyl and alkynyl group is independently unsubstituted or is substituted with 1 or 2 substituents independently selected from C₆ aryl, 5- to 6-membered heteroaryl or with 1-3 halogen substituents;
and wherein each aryl, heteroaryl, and heterocyclyl group is independently unsubstituted or is substituted with 1, 2 or 3 R²⁰ groups, preferably one R²⁰ group.

30. A compound according to claim 26 or claim 27, wherein:
i) R¹ is selected from OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered heterocyclyl;
ii) R² is selected from H, N(R¹⁰)₂, OR¹⁰, halogen, C₁₋₄ alkyl, NR¹⁰—C₁₋₃ alkyl, and NR¹⁰—C₂₋₃ alkynyl, O—C₁₋₃ alkyl, and O—C₂₋₃ alkynyl; phenyl, and 5- to 6-membered heterocyclyl;
wherein each alkyl and alkynyl group is independently unsubstituted or is substituted with 1 or 2 substituents independently selected from C₆ aryl, 5- to 6-membered heteroaryl or with 1-3 halogen substituents;
and wherein each aryl, heteroaryl, and heterocyclyl group is independently unsubstituted or is substituted with 1, 2 or 3 R²⁰ groups, preferably one R²⁰ group.

31. A compound according to claim 26 or claim 27, wherein:
i) R¹ is selected from H, NH₂, OH, OMe, halogen, methyl, NH—C₁₋₃ alkyl, and unsubstituted NH—C₃ alkynyl;
wherein each C₁₋₃ alkyl group is independently unsubstituted or is substituted with 1 or 2 phenyl or pyridinyl groups; wherein said phenyl or pyridinyl groups are each independently unsubstituted or substituted by methyl; and
ii) R² is selected from N(R¹⁰)₂, OH, OMe, halogen, unsubstituted C₁₋₄ alkyl, CF₃, phenyl, piperazinyl, and morpholinyl;
wherein said phenyl, piperazinyl, and morpholinyl groups are independently unsubstituted or substituted by C₁₋₆ alkyl, preferably C₁₋₄ alkyl, more preferably methyl.

32. A compound according to claim 26 or claim 27, wherein:
i) R¹ is selected from OH, OMe, halogen, methyl, NH—C₁₋₃ alkyl, and unsubstituted NH—C₃ alkynyl;
wherein each C₁₋₃ alkyl group is independently unsubstituted or is substituted with 1 or 2 phenyl or pyridinyl groups; wherein said phenyl or pyridinyl groups are each independently unsubstituted or substituted by methyl; and
ii) R² is selected from H, N(R¹⁰)₂, OH, OMe, halogen, unsubstituted C₁₋₄ alkyl, CF₃, phenyl, piperazinyl, and morpholinyl;
wherein said phenyl, piperazinyl, and morpholinyl groups are independently unsubstituted or substituted by C₁₋₆ alkyl, preferably C₁₋₄ alkyl, more preferably methyl.

33. A compound according to claim 28, wherein R¹ and R² are defined as any one of the combinations:

| Compound | | |
|---|---|---|
| number | R¹ | R² |
| 1 | 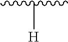 [SMILES: *[H]] | 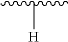 [SMILES: *[H]] |
| 2 | 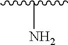 [SMILES: *N] | 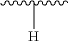 [SMILES: *[H]] |
| 3 | 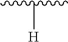 [SMILES: *[H]] | 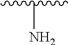 [SMILES: *N] |
| 4 | 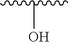 [SMILES: *O] | 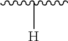 [SMILES: *[H]] |
| 5 | 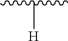 [SMILES: *[H]] | 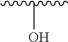 [SMILES: *O] |
| 6 | 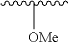 [SMILES: *OC] | 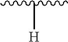 [SMILES: *[H]] |
| 7 | 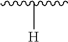 [SMILES: *[H]] | 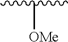 [SMILES: *OC] |
| 8 | 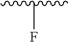 [SMILES: *F] | 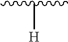 [SMILES: *[H]] |
| 9 | 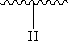 [SMILES: *[H]] | 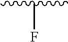 [SMILES: *F] |
| 10 | 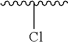 [SMILES: *Cl] | 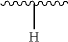 [SMILES: *[H]] |
| 11 | 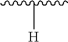 [SMILES: *[H]] | 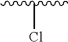 [SMILES: *Cl] |
| 12 | 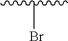 [SMILES: *Br] | 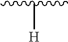 [SMILES: *[H]] |
| 13 | 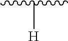 [SMILES: *[H]] | 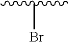 [SMILES: *Br] |
| 14 | 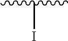 [SMILES: *I] | 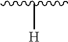 [SMILES: *[H]] |
| 15 | 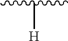 [SMILES: *[H]] | 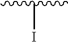 [SMILES: *I] |
| 16 | 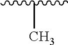 | 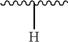 [SMILES: *[H]] |
| 17 | 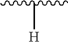 [SMILES: *[H]] | 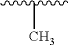 |
| 18 | 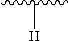 [SMILES: *[H]] | 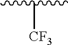 [SMILES: *C(F)(F)F] |
| 19 | 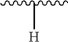 [SMILES: *[H]] | 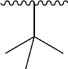 [SMILES: *C(C)(C)C] |
| 20 | 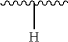 [SMILES: *[H]] | 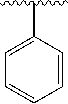 [SMILES: *c1ccccc1] |
| 21 | 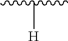 [SMILES: *[H]] | 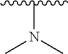 |
| 22 | 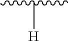 [SMILES: *[H]] | 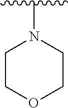 [SMILES: *N1CCOCC1] |
| 23 | 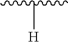 [SMILES: *[H]] | 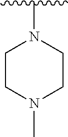 [SMILES: *N1CCN(C)CC1] |
| 24 | 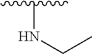 [SMILES: *NCC] | 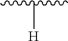 [SMILES: *[H]] |
| 25 | 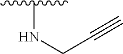 [SMILES: *NCC#C] | 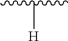 [SMILES: *[H]] |
| 26 | 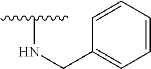 [SMILES: *NCc1ccccc1] | 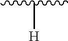 [SMILES: *[H]] |
| 27 | 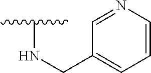 [SMILES: *NCc1cccnc1] | 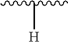 [SMILES: *[H]] |
| 28 | 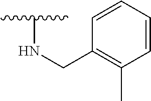 [SMILES: *NCc1ccccc1C] | 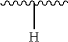 [SMILES: *[H]] |
| 29 | 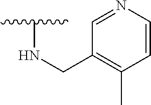 [SMILES: *NCc1cnccc1C] | 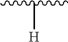 [SMILES: *[H]] |
| 30 | 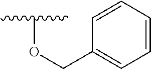 [SMILES: *OCc1ccccc1] | 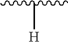 [SMILES: *[H]] |
| 31 | 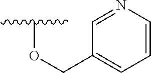 [SMILES: *OCc1cccnc1] | 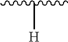 [SMILES: *[H]] |
| 32 | 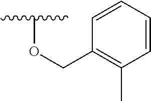 [SMILES: *OCc1ccccc1C] | 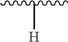 [SMILES: *[H]] |
| 33 | 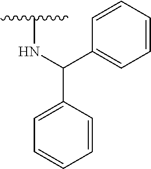 [SMILES: *NC(c1ccccc1)c1ccccc1] | 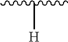 [SMILES: *[H]] |
| 34 | 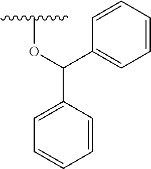 [SMILES: *OC(c1ccccc1)c1ccccc1] | 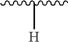 [SMILES: *[H]] |
| 35 | 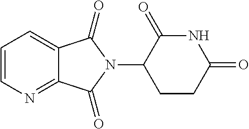 | |
| 36 | 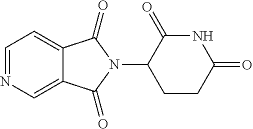 | |
| 37 | 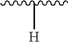 [SMILES: *[H]] | 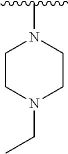 [SMILES: *N1CCN(CC)CC1] |
| 38 | 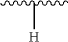 [SMILES: *[H]] | 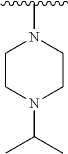 [SMILES: *N1CCN(C(C)C)CC1] |
| 39 | 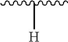 [SMILES: *[H]] | 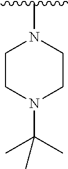 [SMILES: *N1CCN(C(C)(C)C)CC1] |
| 40 | 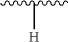 [SMILES: *[H]] | 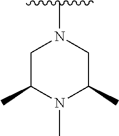 |

34. A compound according to claim 26, wherein the compound is a compound of Formula (Id): 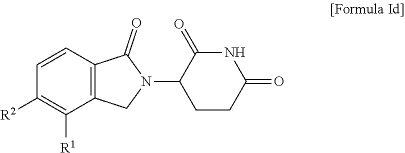
and wherein R¹ and R² are defined as in any one of claims 28 to 33.

35. A pharmaceutical composition comprising a compound according to any one of claims 26 to 34 and a pharmaceutically acceptable excipient or diluent.

36. A kit comprising:
a) a polynucleotide according to claim 22 or claim 23, a vector according to claim 24, or a cell according to claim 25; and
b) a compound according to any one of claims 26 to 34 or as defined in any one of claims 1 to 16.

37. A method for degrading a target protein of interest in a cell, the method comprising:
a) introducing a polynucleotide according to claim 22 or claim 23, or a vector according to claim 24 into the cell such that the cell expresses the polypeptide attached to the target protein of interest;
b) contacting the cell with a compound according to any one of claims 26 to 34 or as defined in any one of claims 1 to 16;
thereby inducing the degradation of the target protein of interest.

38. A method according to claim 37, wherein the polypeptide expressed by the cell is attached to the protein of interest by genetic fusion and/or wherein the polypeptide is attached to the protein of interest by a linker.

39. A compound according to any one of claims 26 to 34, or a pharmaceutical composition according to claim 35, for use in medicine.

40. A compound according to any one of claims 26 to 34, or a pharmaceutical composition according to claim 35, for use in degrading a therapeutic target protein or a cell comprising a therapeutic target protein in a subject;
preferably wherein said target protein is attached to a polypeptide according to claim 17 or 18, or defined in any one of claims 1 to 16.

41. A compound according to any one of claims 26 to 34, or a pharmaceutical composition according to claim 35, for use in the treatment or prevention of cancer in a subject in need thereof.

42. The compound or pharmaceutical composition for use according to any one of claims 39 to 41, wherein the subject has been subject to CAR T-cell therapy, wherein the CAR T-cell therapy comprises:
a) isolation of T-cells from the subject;
b) genetically modifying the isolated T-cells such that isolated T-cells express a chimeric antigen receptor and a polypeptide according to claim 17 or 18, or defined in any one of claims 1 to 16;
c) infusion of the genetically modified T-cells into the subject.

43. The compound or pharmaceutical composition for use according to claim 42, wherein the chimeric antigen receptor and the polypeptide according to claim 17 or 18, or defined in any one of claims 1 to 16 are attached by genetic fusion and/or wherein the polypeptide is attached to the chimeric antigen receptor by a linker.

44. A method comprising genetically modifying T-cells isolated from a subject such that the isolated T-cells express a chimeric antigen receptor and a polypeptide according to claim 17 or 18, or defined in any one of claims 1 to 16.

45. The method according to claim 44, the method further comprising:
a) isolation of the T-cells from the subject; and/or
b) infusion of the genetically modified T-cells into the subject.

46. A polypeptide according to according to claim 17 or 18, or defined in any one of claims 1 to 16, or a polynucleotide according to claim 22 or claim 23, or a vector according to claim 24, for use in medicine.

47. A polypeptide according to claim 17 or 18, or defined in any one of claims 1 to 16, or a polynucleotide according to claim 22 or claim 23, or a vector according to claim 24, for use in the treatment or prevention of cancer in a subject in need thereof.

48. A polypeptide according to claim 17 or 18, or defined in any one of claims 1 to 16, or a polynucleotide according to claim 22 or claim 23, or a vector according to claim 24, for use in a method of CAR T-cell therapy in a subject in need thereof.

49. A method for identifying an orthogonal IMiD compound and polypeptide degron combination, the method comprising screening for increased binding affinity of one or more compounds according to any one of claims 26 to 34 or as defined in any one of claims 1 to 16 against a polypeptide variant of SEQ ID NO: 2 or SEQ ID NO: 3 relative to a polypeptide sequence consisting of or comprising the polypeptide sequence of SEQ ID NO: 2 or SEQ ID NO: 3, thereby identifying an orthogonal IMiD compound and polypeptide degron combination.

50. The method according to claim 49, wherein the polypeptide variant is as defined in claim 17 or 18 or defined in any one of claims 1 to 16, and/or wherein the compound is as defined in any one of claims 26 to 34 or as defined in any one of claims 1 to 16.

51. Use of a compound as defined according to any one of claims 26 to 34, or as defined in any one of claims 1 to 16, in selectively degrading a target protein of interest in a composition comprising the target protein of interest and a polypeptide of SEQ ID NO: 2 and/or 3.
